(19) United States
(12) Patent Application Publication
(10) Pub. No.: US 2024/0344061 A1
(43) Pub. Date: Oct. 17, 2024

(54) COMPOSITIONS AND METHODS FOR MODULATING HEPATOCYTE NUCLEAR FACTOR 4-ALPHA (HNF4alpha) GENE EXPRESSION

(71) Applicant: Omega Therapeutics, Inc., Cambridge, MA (US)

(72) Inventors: Jesse Jerome Smith, Waltham, MA (US); Jodi Michelle Kennedy, Dedham, MA (US); Jeremiah D. Farelli, Marblehead, MA (US); Kendrick Alan Goss, Lexington, MA (US); Adam Walter Scheidegger, Somerville, MA (US); Yoseph Kassa, Cambridge, MA (US); Christian Wessel Cobaugh, Newton, MA (US); Timsi Rao, Allston, MA (US)

(73) Assignee: Omega Therapeutics, Inc., Cambridge, MA (US)

(21) Appl. No.: 18/637,630

(22) Filed: Apr. 17, 2024

Related U.S. Application Data
(63) Continuation of application No. 17/029,820, filed on Sep. 23, 2020, now Pat. No. 11,987,791.
(60) Provisional application No. 62/904,178, filed on Sep. 23, 2019.

(51) Int. Cl. C12N 15/11; C12N 9/22

(52) U.S. Cl. CPC C12N 15/11; C12N 9/22; C07K 2319/80; C07K 2319/81; C12N 2310/20

(57) ABSTRACT

The present invention provides agents and compositions for modulating expression (e.g., enhanced or reduced expression) of a hepatocyte nuclear factor 4 alpha (HNF4α) gene by targeting an HNF4α expression control region and methods of use thereof for treating an HNF4α associated disorder, e.g., cirrhosis.

### CROSS-REFERENCE TO RELATED APPLICATIONS

This application is a continuation application of U.S. patent application Ser. No. 17/029,820, filed on Sep. 23, 2020, which claims the benefit of priority to U.S. Provisional Application No. 62/904,178, filed on Sep. 23, 2019. The entire contents of each of the foregoing applications are incorporated herein by reference.

### SEQUENCE LISTING

The instant application contains a Sequence Listing which has been submitted electronically in XML format and is hereby incorporated by reference in its entirety. Said XML copy, created on Feb. 21, 2024, is named 131717-00104_SL.xml and is 9,794,631 bytes in size.

### BACKGROUND OF THE INVENTION

Master regulators are proteins, such as transcription factors, with the ability to influence the expression of a network of genes related by cell type, organ system, or response to a stimulus.

One example of a master regulator is the transcription factor Hepatocyte Nuclear Factor 4-alpha (HNF4α). HNF4α is expressed for the first time in terminally differentiated liver cells, late in embryonic development. HNF4α controls the expression of proteins necessary for the normal function of hepatocytes and other cell types in the liver (Li, et al. (2000) Genes and Devel 14:464-474). In addition, many of these proteins are secreted by the liver cells and contribute to health systemically. For example, proteins such as albumin are required to transport nutrients, hormones, lipids, and small molecule drugs in the circulation. In fibrotic liver disease, HNF4α is dysregulated and, as a result, gene expression in its network declines significantly or stops (Guzman-Lepe, et al. (2018) Hepatol Comm 2(5):582). This dysregulation of the network contributes to the pathology of liver failure in the organ itself, and to co-morbidities throughout the patient.

Recently, Nishikawa et al. (2015) demonstrated that transgenic expression of HNF4α in a rat model of cirrhosis led to the restoration of gene expression throughout the HNF4α network, restored hepatocyte function, and improved health of the animal. The transgene was delivered with an adeno-associated virus (AAV). However, transgene expression from AAV delivery does not allow subtle control or temporary modification of the expression of genes already in the genome. Once modified with AAV, the affected cells lose the ability to respond to changing conditions in the organ and body in a nimble physiologically meaningful way.

Accordingly there is a need in the art for temporary and labile effectors that offer greater control and the ability to restore cells and tissues to a nascent “normal” state and, thus, treat HNF4α-associated disease, such as fibrotic liver disease, e.g., cirrhosis.

### SUMMARY OF THE INVENTION

The present invention provides agents and compositions for modulating the expression (e.g., enhancing or reducing expression) of a hepatocyte nuclear factor 4 alpha (HNF4α) gene by targeting an HNF4α expression control region. The HNF4α gene may be in a cell, e.g., a mammalian cell, such as a mammalian somatic cell, e.g., a human somatic cell. The present invention also provides methods of using the agents and compositions of the invention for modulating the expression of an HNF4α gene or for treating a subject who would benefit from modulating the expression of an HNF4α gene, e.g., a subject suffering or prone to suffering from an HNF4α-associated disease.

Accordingly, in one aspect, the present invention provides a site-specific disrupting agent, comprising a site-specific HNF4α targeting moiety which targets an HNF4α expression control region.

In some embodiments, the site-specific HNF4α targeting moiety comprises a polymeric molecule.

The polymeric molecule may comprise a polyamide, a polynucleotide, a polynucleotide encoding a DNA-binding domain, or fragment thereof, that specifically binds to the HNF4α expression control region, or a peptide nucleic acid (PNA).

In some embodiments, the expression control region comprises an HNF4α-specific transcriptional control element.

In some embodiments, the transcriptional control element comprises an HNF4α promoter, such as the nucleotide sequence of HNF4α promoter 1, or a fragment thereof, or the nucleotide sequence of HNF4α promoter 2, or a fragment thereof.

In some embodiments, the transcriptional control element comprises a transcriptional enhancer.

In some embodiments, the transcriptional control element comprises a transcriptional repressor.

In some embodiments, the site-specific HNF4α disrupting agent comprises a nucleotide sequence having at least 85%, 86%, 87%, 88%, 89%, 90%, 91%, 92%, 93%, 94%, 95%, 96%, 97%, 98%, 99%, or 100% nucleotide identity to the entire nucleotide sequence of any of the nucleotide sequences in any one of Tables 2, 3, 4, and 9.

In some embodiments, the site-specific HNF4α disrupting agent comprises a polynucleotide encoding a DNA-binding domain of a Transcription activator-like effector (TALE) polypeptide or a zinc finger (ZNF) polypeptide, or fragment thereof, that specifically binds to the HNF4α expression control region.

In one embodiment, the DNA-binding domain of the TALE or ZNF polypeptide comprises an amino acid sequence having at least about 85%, 86%, 87%, 88%, 89%, 90%, 91%, 92%, 93%, 94%, 95%, 96%, 97%, 98%, 99%, or 100% amino acid identity to the entire amino acid sequence of any one of the amino acid sequences listed in column 5 of Table 6A or column 4 of Table 10. In one embodiment, DNA-binding domain of the TALE or ZNF polypeptide comprises an amino acid sequence having at least about 85%, 86%, 87%, 88%, 89%, 90%, 91%, 92%, 93%, 94%, 95%, 96%, 97%, 98%, 99%, or 100% amino acid identity to the entire amino acid sequence of ZF5.3. In one embodiment, DNA-binding domain of the TALE or ZNF polypeptide comprises an amino acid sequence having at least about 85%, 86%, 87%, 88%, 89%, 90%, 91%, 92%, 93%, 94%, 95%, 96%, 97%, 98%, 99%, or 100% amino acid identity to the entire amino acid sequence of ZF7. In one embodiment, DNA-binding domain of the TALE or ZNF polypeptide comprises an amino acid sequence having at least about 85%, 86%, 87%, 88%, 89%, 90%, 91%, 92%, 93%, 94%, 95%, 96%, 97%, 98%, 99%, or 100% amino acid identity to the entire amino acid sequence of ZF14. In one embodiment, DNA-binding domain of the TALE or ZNF polypeptide comprises an amino acid sequence having at least about 85%, 86%, 87%, 88%, 89%, 90%, 91%, 92%, 93%, 94%, 95%, 96%, 97%, 98%, 99%, or 100% amino acid identity to the entire amino acid sequence of ZF15.

In some embodiments, the expression control region comprises one or more HNF4α-associated anchor sequences within an anchor sequence-mediated conjunction comprising a first and a second HNF4α-associated anchor sequence.

In some embodiments, the anchor sequence comprises a CCCTC-binding factor (CTCF) binding motif.

In some embodiments, the anchor sequence-mediated conjunction comprises one or more transcriptional control elements internal to the conjunction.

In some embodiments, the anchor sequence-mediated conjunction comprises one or more transcriptional control elements external to the conjunction.

In some embodiments, the first and/or the second anchor sequence is located within about 500 kb of the transcriptional control element.

In some embodiments, the first and/or the second anchor sequence is located within 300 kb of the transcriptional control element.

In some embodiments, the site-specific HNF4α disrupting agent comprises a nucleotide modification.

The present invention also provides vectors, such as viral expression vectors and cells comprising the site-specific HNF4α disrupting agents of the invention as well as the vectors of the invention.

In some embodiments, the site-specific HNF4α disrupting agents of the invention are present in a composition, such as a pharmaceutical composition.

In some embodiments, the pharmaceutical composition comprises a lipid formulation.

In some embodiments, the lipid formulation comprises one or more cationic lipids, one or more non-cationic lipids, one or more cholesterol-based lipids, or one or more PEG-modified lipids, or combinations of any of the foregoing.

In some embodiments, the pharmaceutical composition comprises a lipid nanoparticle.

In another aspect, the present invention provides a site-specific HNF4α disrupting agent, comprising a nucleic acid molecule encoding a fusion protein, the fusion protein comprising a site-specific HNF4α targeting moiety which targets an HNF4α expression control region and an effector molecule.

In some embodiments, the site-specific HNF4α targeting moiety comprises a polynucleotide encoding a DNA-binding domain of a Transcription activator-like effector (TALE) polypeptide or a zinc finger (ZNF) polypeptide, or fragment thereof, that specifically binds to the HNF4α expression control region.

In one embodiment, the DNA-binding domain of the TALE comprises an amino acid sequence having at least 85%, 86%, 87%, 88%, 89%, 90%, 91%, 92%, 93%, 94%, 95%, 96%, 97%, 98%, 99%, or 100% amino acid identity to the entire amino acid sequence of an amino acid sequence selected from the amino acid sequences listed in column 5 of Table 6A or column 4 of Table 10.

In some embodiments, the effector molecule comprises a polypeptide or a nucleic acid molecule encoding a polypeptide.

In some embodiments, the fusion protein comprises a peptide-nucleic acid fusion.

In some embodiments, the effector is selected from the group consisting of a nuclease, a physical blocker, an epigenetic recruiter, and an epigenetic CpG modifier, and combinations of any of the foregoing.

In some embodiments, the effector comprises a CRISPR associated protein (Cas) polypeptide or nucleic acid molecule encoding the Cas polypeptide.

In some embodiments, the Cas polypeptide is an enzymatically inactive Cas polypeptide.

In some embodiments, the Cas polypeptide comprises a catalytically active domain of human exonuclease 1 (hEXO1).

In some embodiments, the epigenetic recruiter comprises a transcriptional enhancer or a transcriptional repressor.

In one embodiment, the transcriptional enhancer is a VPR (VP64-p65-Rta).

In one embodiment, the VPR comprises an amino acid sequence having at least about 85%, 86%, 87%, 88%, 89%, 90%, 91%, 92%, 93%, 94%, 95%, 96%, 97%, 98%, 99%, or 100% amino acid identity to the entire amino acid sequence of

(SEQ ID NO: 66)
DALDDFDLDMLGSDALDDFDLDMLGSDALDDFDLDMLGSDALDD
FDLDMLSGGPKKKRKVGSQYLPDTDDRHRIEEKRKRTYETFKSI
MKKSPFSGPTDPRPPPRRIAVPSRSSASVPKPAPQPYPFTSSLS
TINYDEFPTMVFPSGQISQASALAPAPPQVLPQAPAPAPAPAMV
SALAQAPAPVPVLAPGPPQAVAPPAPKPTQAGEGTLSEALLQLQ
FDDEDLGALLGNSTDPAVFTDLASVDNSEFQQLLNQGIPVAPHT
TEPMLMEYPEAITRLVTGAQRPPDPAPAPLGAPGLPNGLLSGDE
DFSSIADMDFSALLGSGSGSRDSREGMFLPKPEAGSAISDVFEG
REVCQPKRLRPFHPPGSPWANRPLPASLAPTPTGPVHEPVGSLT
PAPVPQPLDPAPAVTPEASHLLEDPDEETSQAVKALREMADTVI
PQKEEAAICGQMDLSHPPPRGHLDELTTTLESMTEDLNLDSPLT
PELNEILDTFLNDECLLHAMHISTGLSIFDTSLF.

In one embodiment, the transcriptional enhancer comprises two, three, four, or five VPRs.

In one embodiment, the transcriptional enhancer is a p300.

In one embodiment, the p300 comprises an amino acid sequence having at least about 85% 85%, 86%, 87%, 88%, 89%, 90%, 91%, 92%, 93%, 94%, 95%, 96%, 97%, 98%, 99%, or 100% identity to the entire amino acid sequence of

(SEQ ID NO: 67)
IFKPEELRQALMPTLEALYRQDPESLPFRQPVDPQLLGIPDYF
DIVKSPMDLSTIKRKLDTGQYQEPWQYVDDIWLMFNNAWLYNR
KTSRVYKYCSKLSEVFEQEIDPVMQSLGYCCGRKLEFSPQTLC
CYGKQLCTIPRDATYYSYQNRYHFCEKCFNEIQGESVSLGDDP
SQPQTTINKEQFSKRKNDTLDPELFVECTECGRKMHQICVLHH
EIIVVPAGFVCDGCLKKSARTRKENKFSAKRLPSTRLGTFLEN
RVNDFLRRQNHPESGEVTVRVVHASDKTVEVKPGMKARFVDSG
EMAESFPYRTKALFAFEEIDGVDLCFFGMHVQEYGSDCPPPNQ
RRVYISYLDSVHFFRPKCLRTAVYHEILIGYLEYVKKLGYTTG
HIVVACPPSEGDDYIFHCHPPDQKIPKPKRLQEWYKKMLDKAV
SERIVHDYKDIFKQATEDRLTSAKELPYFEGDFWPNVLEESIK
ELEQEEEERKREENTSNESTDVTKGDSKNAKKKNNKKTSKNKS
SLSRGNKKKPGMPNVSNDLSQKLYATMEKHKEVFFVIRLIAGP
PAANSLPPIVDPDPLIPCDLMDGRDAFLTLARDKHLEFSSLRR
AQWSTMCMLVELHTQSQD.

In some embodiments, the epigenetic CpG modifier comprises a DNA methylase, a DNA demethylase, a histone modifying agent, or a histone deacetylase.

In some embodiments, the effector molecule comprises a zinc finger polypeptide.

In some embodiments, the effector molecule comprises a Transcription activator-like effector nuclease (TALEN) polypeptide.

In some embodiments, the site-specific HNF4α disrupting agent further comprises a second nucleic acid molecule encoding a second fusion protein, wherein the second fusion comprises a second site-specific HNF4α targeting moiety which targets a second HNF4α expression control region and a second effector molecule, wherein the second HNF4α expression control region is different than the HNF4α expression control region.

In one embodiment, the HNF4α expression control region comprises a ZF5 target sequence GGCGGGGGACCGATTAACCAT (SEQ ID NO: 118) and the second HNF4α expression control region comprises a ZF7 target sequence ACTGAACATCGGTGAGTTAGG (SEQ ID NO: 126).

In one embodiment, the second effector is different than the first effector.

In one embodiment, the second effector is the same as the first effector.

In one embodiment, the fusion protein and the second fusion protein are operably linked.

In one embodiment, the fusion protein and the second fusion protein comprise an amino acid sequence that has at least about 85%, 86%, 87%, 88%, 89%, 90%, 91%, 92%, 93%, 94%, 95%, 96%, 97%, 98%, 99%, or 100% amino acid sequence identity to the entire amino acid sequence of a polypeptide selected from the group consisting of ZF5.3-VPR-tPT2a-ZF7-VPR; ZF7-VPR-tPT2a-ZF5.3-VPR; ZF5.3-VPR-tPT2a-ZF7-p300; and ZF7-p300-tPT2a-ZF5.3-VPR.

In one embodiment, the fusion protein comprises an amino acid sequence having at least about 85%, 86%, 87%, 88%, 89%, 90%, 91%, 92%, 93%, 94%, 95%, 96%, 97%, 98%, 99%, or 100% amino acid sequence identity to the entire amino acid sequence of ZF5.3-VPR.

In one embodiment, the fusion protein is encoded by a polynucleotide comprising a nucleotide sequence having at least about 85%, 86%, 87%, 88%, 89%, 90%, 91%, 92%, 93%, 94%, 95%, 96%, 97%, 98%, 99%, or 100% nucleotide sequence identity to the entire nucleotide sequence of a polynucleotide selected from the group consisting of ZF5-VPR mRNA, ZF5.1-VPR mRNA, ZF5.2-VPR mRNA, ZF5.3-VPR mRNA, ZF5.4-VPR mRNA, ZF5.5-VPR mRNA, and ZF5.6-VPR mRNA.

In one aspect, the present invention provides a site-specific HNF4α disrupting agent. The disrupting agent includes a nucleic acid molecule encoding a fusion protein, wherein the fusion protein comprises an amino acid sequence having at least about 85%, 86%, 87%, 88%, 89%, 90%, 91%, 92%, 93%, 94%, 95%, 96%, 97%, 98%, 99%, or 100% amino acid identity to the entire amino acid sequence of a polypeptide selected from the group consisting of ZF1-VPR, ZF2-VPR, ZF3-VPR, ZF4-VPR, ZF5-VPR, ZF5.3-VPR, ZF6-VPR, ZF7-VPR, ZF8-VPR, ZF9-VPR, ZF10-VPR, ZF11-VPR, ZF12-VPR, ZF13-VPR, ZF14-VPR, and ZF15-VPR.

In one embodiment, the polypeptide is selected from the group consisting of ZF5-VPR, ZF5.3-VPR, ZF7-VPR, ZF10-VPR, ZF14-VPR, and ZF15-VPR.

In one embodiment, the polypeptide is ZF5.3-VPR.

The present invention also provides vectors, such as viral expression vectors and cells comprising the site-specific HNF4α disrupting agents of the invention as well as the vectors of the invention.

In some embodiments, the site-specific HNF4α disrupting agents of the invention are present in a composition, such as a pharmaceutical composition.

In some embodiments, the pharmaceutical composition comprises a lipid formulation.

In some embodiments, the lipid formulation comprises one or more cationic lipids, one or more non-cationic lipids, one or more cholesterol-based lipids, or one or more PEG-modified lipids, or combinations of any of the foregoing.

In some embodiments, the pharmaceutical composition comprises a lipid nanoparticle.

In one aspect, the present invention provides a method of modulating expression of hepatocyte nuclear factor 4 alpha-(HNF4α) in a cell. The method includes contacting the cell with a site-specific HNF4α disrupting agent, the disrupting agent comprising a site-specific HNF4α targeting moiety which targets an HNF4α expression control region, and an effector molecule, thereby modulating expression of HNF4α in the cell.

The modulation of expression may be enhanced expression of HNF4α in the cell or reduced expression of HNF4α in the cell.

In some embodiments, the site-specific HNF4α targeting moiety comprises a polymeric molecule.

The polymeric molecule may comprise a polyamide, a polynucleotide, a peptide nucleic acid (PNA).

In some embodiments, the expression control region comprises an HNF4α-specific transcriptional control element.

In some embodiments, the transcriptional control element comprises an HNF4α promoter, such as the nucleotide sequence of HNF4α promoter 1, or a fragment thereof, or the nucleotide sequence of HNF4α promoter 2, or a fragment thereof.

In some embodiments, the transcriptional control element comprises a transcriptional enhancer.

In some embodiments, the transcriptional control element comprises a transcriptional repressor.

In some embodiments, the site-specific HNF4α disrupting agent comprises a nucleotide sequence having at least 85%, 86%, 87%, 88%, 89%, 90%, 91%, 92%, 93%, 94%, 95%, 96%, 97%, 98%, 99%, or 100% nucleotide identity to the entire nucleotide sequence of any of the nucleotide sequences in any one of Tables 2, 3, 4, and 9.

In some embodiments, the site-specific HNF4α disrupting agent comprises a polynucleotide encoding a DNA-binding domain of a Transcription activator-like effector (TALE) polypeptide or a zinc finger (ZNF) polypeptide, or fragment thereof, that specifically binds to the HNF4α expression control region.

In some embodiments, the DNA-binding domain of the TALE or ZNF comprises an amino acid sequence having at least 85%, 86%, 87%, 88%, 89%, 90%, 91%, 92%, 93%, 94%, 95%, 96%, 97%, 98%, 99%, or 100% amino acid identity to the entire amino acid sequence of an amino acid sequence selected from the amino acid sequences listed in column 5 of Table 6A or column 4 of Table 10.

In some embodiments, the expression control region comprises one or more HNF4α-associated anchor sequences within an anchor sequence-mediated conjunction comprising a first and a second HNF4α-associated anchor sequence.

In some embodiments, the anchor sequence comprises a CCCTC-binding factor (CTCF) binding motif.

In some embodiments, the anchor sequence-mediated conjunction comprises one or more transcriptional control elements internal to the conjunction.

In some embodiments, the anchor sequence-mediated conjunction comprises one or more transcriptional control elements external to the conjunction.

In some embodiments, the first and/or the second anchor sequence is located within about 500 kb of the transcriptional control element.

In some embodiments, the first and/or the second anchor sequence is located within 300 kb of the transcriptional control element.

In some embodiments, the site-specific HNF4α disrupting agent comprises a nucleotide modification.

In some embodiments, the effector molecule comprises a polypeptide.

In some embodiments, the polypeptide comprises a nucleic acid molecule encoding a fusion protein comprising the site-specific HNF4α targeting moiety which targets an HNF4α expression regulatory region, and the effector molecule.

In some embodiments, the fusion protein comprises a peptide-nucleic acid fusion molecule.

In some embodiments, the effector is selected from the group consisting of a nuclease, a physical blocker, an epigenetic recruiter, and an epigenetic CpG modifier, and combinations of any of the foregoing.

In some embodiments, the effector comprises a CRISPR associated protein (Cas) polypeptide or nucleic acid molecule encoding the Cas polypeptide.

In some embodiments, the Cas polypeptide is an enzymatically inactive Cas polypeptide.

In some embodiments, the Cas polypeptide further comprises a catalytically active domain of human exonuclease 1 (hEXO1).

In some embodiments, the epigenetic recruiter comprises a transcriptional enhancer or a transcriptional repressor.

In some embodiments, the transcriptional enhancer is a VPR.

In some embodiments, the VPR comprises an amino acid sequence having at least about 85%, 86%, 87%, 88%, 89%, 90%, 91%, 92%, 93%, 94%, 95%, 96%, 97%, 98%, 99%, or 100% amino acid identity to the entire amino acid sequence of

(SEQ ID NO: 66)
DALDDFDLDMLGSDALDDFDLDMLGSDALDDFDLDMLGSDALD
DFDLDMLSGGPKKKRKVGSQYLPDTDDRHRIEEKRKRTYETFK
SIMKKSPFSGPTDPRPPPRRIAVPSRSSASVPKPAPQPYPFTS
SLSTINYDEFPTMVFPSGQISQASALAPAPPQVLPQAPAPAPA
PAMVSALAQAPAPVPVLAPGPPQAVAPPAPKPTQAGEGTLSEA
LLQLQFDDEDLGALLGNSTDPAVFTDLASVDNSEFQQLLNQGI
PVAPHTTEPMLMEYPEAITRLVTGAQRPPDPAPAPLGAPGLPN
GLLSGDEDFSSIADMDFSALLGSGSGSRDSREGMFLPKPEAGS
AISDVFEGREVCQPKRLRPFHPPGSPWANRPLPASLAPTPTGP
VHEPVGSLTPAPVPQPLDPAPAVTPEASHLLEDPDEETSQAVK
ALREMADTVIPQKEEAAICGQMDLSHPPPRGHLDELTTTLESM
TEDLNLDSPLTPELNEILDTFLNDECLLHAMHISTGLSIFDTSLF.

In some embodiments, the transcriptional enhancer comprises two, three, four, or five VPRs.

In some embodiments, the transcriptional enhancer is a p300.

In some embodiments, the p300 has an amino acid sequence having at least about 85%, 86%, 87%, 88%, 89%, 90%, 91%, 92%, 93%, 94%, 95%, 96%, 97%, 98%, 99%, or 100% amino acid identity to the entire amino acid sequence of

(SEQ ID NO: 67)
IFKPEELRQALMPTLEALYRQDPESLPFRQPVDPQLLGIPDYFDIVKSPM
DLSTIKRKLDTGQYQEPWQYVDDIWLMFNNAWLYNRKTSRVYKYCSKLSE
VFEQEIDPVMQSLGYCCGRKLEFSPQTLCCYGKQLCTIPRDATYYSYQNR
YHFCEKCFNEIQGESVSLGDDPSQPQTTINKEQFSKRKNDTLDPELFVEC
TECGRKMHQICVLHHEIIWPAGFVCDGCLKKSARTRKENKFSAKRLPSTR
LGTFLENRVNDFLRRQNHPESGEVTVRVVHASDKTVEVKPGMKARFVDSG
EMAESFPYRTKALFAFEEIDGVDLCFFGMHVQEYGSDCPPPNQRRVYISY
LDSVHFFRPKCLRTAVYHEILIGYLEYVKKLGYTTGHIWACPPSEGDDYI
FHCHPPDQKIPKPKRLQEWYKKMLDKAVSERIVHDYKDIFKQATEDRLTS
AKELPYFEGDFWPNVLEESIKELEQEEEERKREENTSNESTDVTKGDSKN
AKKKNNKKTSKNKSSLSRGNKKKPGMPNVSNDLSQKLYATMEKHKEVFFV
IRLIAGPAANSLPPIVDPDPLIPCDLMDGRDAFLTLARDKHLEFSSLRRA
QWSTMCMLVELHTQSQD.

In some embodiments, the epigenetic CpG modifier comprises a DNA methylase, a DNA demethylase, a histone modifying agent, or a histone deacetylase.

In some embodiments, the effector molecule comprises a zinc finger polypeptide.

In some embodiments, the effector molecule comprises a Transcription activator-like effector nuclease (TALEN) polypeptide.

In some embodiments, the fusion protein comprises an enzymatically inactive Cas polypeptide and an epigenetic recruiter polypeptide.

In some embodiments, the fusion protein comprises an enzymatically Cas polypeptide and an epigenetic CpG modifier polypeptide.

In some embodiments, the site-specific HNF4α disrupting agent comprises a second nucleic acid molecule encoding a second fusion protein, wherein the second fusion protein comprises a second site-specific HNF4α targeting moiety which targets a second HNF4α expression control region and a second effector molecule, wherein the second HNF4α expression control region is different than the HNF4α expression control region.

In some embodiments, the HNF4α expression control region comprises a ZF5 target sequence GGCGGGGGACCGATTAACCAT (SEQ ID NO: 118) and the second HNF4α expression control region comprises a ZF7 target sequence ACTGAACATCGGTGAGTTAGG (SEQ ID NO: 126).

In some embodiments, the second effector is different than the effector.

In some embodiments, the second effector is the same as the effector.

In some embodiments, the fusion protein and the second fusion protein are operably linked.

In some embodiments, the fusion protein and the second fusion protein comprise an amino acid sequence that has at least about 85%, 86%, 87%, 88%, 89%, 90%, 91%, 92%, 93%, 94%, 95%, 96%, 97%, 98%, 99%, or 100% sequence identity to the entire amino acid sequence of a polypeptide selected from ZF5.3-VPR-tPT2a-ZF7-VPR protein, ZF7-VPR-tPT2a-ZF5.3-VPR protein, ZF5.3-VPR-tPT2a-ZF7-p300 protein, and ZF7-p300-tPT2a-ZF5.3-VPR protein.

In some embodiments, the fusion protein comprises an amino acid sequence having at least 85%, 86%, 87%, 88%, 89%, 90%, 91%, 92%, 93%, 94%, 95%, 96%, 97%, 98%, 99%, or 100% sequence identity to the entire amino acid sequence of ZF5.3-VPR protein.

In some embodiments, the fusion protein is encoded by a polynucleotide having a sequence that has at least about 85%, 86%, 87%, 88%, 89%, 90%, 91%, 92%, 93%, 94%, 95%, 96%, 97%, 98%, 99%, or 100% sequence identity to the entire nucleotide sequence of a polynucleotide selected from the group consisting of ZF5-VPR mRNA, ZF5.1-VPR mRNA, ZF5.2-VPR mRNA, ZF5.3-VPR mRNA, ZF5.4-VPR mRNA, ZF5.5-VPR mRNA, and ZF5.6-VPR mRNA.

In some embodiments, the administration of the site-specific HNF4α disrupting agent and the second site-specific HNF4α disrupting agent has a synergistic effect in modulating the expression of HNF4α.

In some embodiments, the HNF4α expression control region comprises a ZF5 target sequence GGCGGGGGACCGATTAACCAT (SEQ ID NO: 118) and the second HNF4α expression control region comprises a sequence selected from ZF7 target sequence ACTGAACATCGGTGAGTTAGG (SEQ ID NO: 126), ZF10 target sequence CCTGCAGCCCCGCCCAGCCTA (SEQ ID NO: 138), ZF14 target sequence GGAGGGGTGGGGGTTAATGGT (SEQ ID NO: 154), and ZF15 target sequence GAAGGGGTGGAGGCTCTGCCG (SEQ ID NO: 158).

In some embodiments, the HNF4α expression control region comprises a ZF5 target sequence GGCGGGGGACCGATTAACCAT (SEQ ID NO: 118) and the second HNF4α expression control region comprises a ZF7 target sequence ACTGAACATCGGTGAGTTAGG (SEQ ID NO: 126).

In some embodiments, the fusion protein comprises a sequence having at least 85%, 86%, 87%, 88%, 89%, 90%, 91%, 92%, 93%, 94%, 95%, 96%, 97%, 98%, 99%, or 100% sequence identity to the entire amino acid sequence of ZF5-VPR, and the second fusion protein comprises a sequence having at least 85%, 86%, 87%, 88%, 89%, 90%, 91%, 92%, 93%, 94%, 95%, 96%, 97%, 98%, 99%, or 100% sequence identity to the entire amino acid sequence of a polypeptide selected from ZF7-VPR, ZF10-VPR, ZF14-VPR, and ZF15-VPR.

In some embodiments, the fusion protein comprises an amino acid sequence having at least about 85%, 86%, 87%, 88%, 89%, 90%, 91%, 92%, 93%, 94%, 95%, 96%, 97%, 98%, 99%, or 100% identity to the entire amino acid sequence of a polypeptide selected from the group consisting of ZF1-VPR, ZF2-VPR, ZF3-VPR, ZF4-VPR, ZF5-VPR, ZF5.3-VPR, ZF6-VPR, ZF7-VPR, ZF8-VPR, ZF9-VPR, ZF10-VPR, ZF11-VPR, ZF12-VPR, ZF13-VPR, ZF14-VPR, and ZF15-VPR.

In some embodiments, the polypeptide is selected from the group consisting of ZF5-VPR, ZF5.3-VPR, ZF7-VPR, ZF10-VPR, ZF14-VPR, and ZF15-VPR.

In some embodiments, the polypeptide is ZF5.3-VPR.

In some embodiments, the site-specific disrupting agent, the effector, or both the site-specific disrupting agent and the effector are present in a vector, such as a viral expression vector.

In some embodiments, the site-specific disrupting agent and the effector are present in the same vector.

In some embodiments, the site-specific disrupting agent and the effector are present in different vectors.

In some embodiments, the site-specific disrupting agent, the effector, or both the site-specific disrupting agent and the effector are present in a composition.

In some embodiments, the site-specific disrupting agent and the effector are present in the same composition.

In some embodiments, the site-specific disrupting agent and the effector are present in different compositions.

In some embodiments, the composition comprises a pharmaceutical composition.

In some embodiments, the pharmaceutical composition comprises a lipid formulation.

In some embodiments, the lipid formulation comprises one or more cationic lipids, one or more non-cationic lipids, one or more cholesterol-based lipids, or one or more PEG-modified lipids, or combinations of any of the foregoing.

In some embodiments, the pharmaceutical composition comprises a lipid nanoparticle.

In some embodiments, the cell is a mammalian cell, such as a somatic cell or a primary cell.

In some embodiments, the contacting is performed in vitro.

In some embodiments, the contacting is performed in vivo.

In some embodiments, the contacting is performed ex vivo.

In some embodiments, the methods of the invention further comprise administering the cell to a subject.

In some embodiments, the cell is within a subject.

In some embodiments, the subject has an HNF4α-associated disease.

In some embodiments, the HNF4α-associated disease is selected from the group consisting of fatty liver (steatosis), nonalcoholic steatohepatitis (NASH), cirrhosis of the liver, accumulation of fat in the liver, inflammation of the liver, hepatocellular necrosis, liver fibrosis, and nonalcoholic fatty liver disease (NAFLD), polycystic kidney disease, inflammatory bowel disease (IBD), and MODY I.

In another aspect, the present invention provides a method for treating a subject having an HNF4α-associated disease. The method includes administering to the subject a therapeutically effective amount of the site-specific HNF4α disrupting agent, the disrupting agent comprising a site-specific HNF4α targeting moiety which targets an HNF4α expression control region, and an effector molecule, thereby treating the subject.

In some embodiments, the HNF4α-associated disease is hepatocellular cancer and the site-specific HNF4α disrupting agent reduces expression of HNF4α in the subject.

In some embodiments, the HNF4α-associated disease is selected from the group consisting of fatty liver (steatosis), nonalcoholic steatohepatitis (NASH), cirrhosis of the liver, accumulation of fat in the liver, inflammation of the liver, hepatocellular necrosis, liver fibrosis, and nonalcoholic fatty liver disease (NAFLD) and the site-specific HNF4α disrupting agent enhances expression of HNF4α in the subject.

In some embodiments, the site-specific HNF4α disrupting agent and the effector molecule are administered to the subject concurrently.

In some embodiments, the site-specific HNF4α disrupting agent and the effector molecule are administered to the subject sequentially.

In some embodiments, the effector molecule is administered to the subject prior to administration of the site-specific HNF4α disrupting agent.

In some embodiments, the site-specific HNF4α disrupting agent is administered to the subject prior to administration of the effector molecule.

In one aspect, the present invention provides a site-specific HNF4α disrupting agent, comprising a site-specific HNF4α targeting moiety which targets an HNF4α expression control region, wherein the HNFα expression control region comprises the nucleotide sequence of any one of the nucleotide sequences listed in column 3 of Table 1 or column 4 of Table 10.

In one embodiment, the site-specific HNF4α targeting moiety comprises a nucleotide sequence having at least 85%, 86%, 87%, 88%, 89%, 90%, 91%, 92%, 93%, 94%, 95%, 96%, 97%, 98%, 99%, or 100% nucleotide identity to the entire nucleotide sequence of any of the nucleotide sequences in any one of Table 2, 3, 4, and 9.

In one embodiment, the site-specific HNF4α targeting moiety comprises a polymeric molecule comprising a polynucleotide encoding a DNA-binding domain of a Transcription activator-like effector (TALE) polypeptide or a zinc finger (ZNF) polypeptide, or fragment thereof, that specifically binds to the HNF4α expression control region.

In one embodiment, the DNA-binding domain of the TALE or ZNF polypeptide comprises an amino acid sequence having at least about 85%, 86%, 87%, 88%, 89%, 90%, 91%, 92%, 93%, 94%, 95%, 96%, 97%, 98%, 99%, or 100% amino acid identity to the entire amino acid sequence of any one of the amino acid sequences listed in column 5 of Table 6A or column 4 of Table 10.

In one embodiment, the HNFα expression control region comprises a nucleotide sequence of a ZF5 target sequence GGCGGGGGACCGATTAACCAT (SEQ ID NO: 118).

In one embodiment, the HNFα expression control region comprises a nucleotide sequence of a ZF7 target sequence ACTGAACATCGGTGAGTTAGG (SEQ ID NO: 126).

In one aspect, the present invention provides a site-specific HNF4α disrupting agent, comprising a polynucleotide encoding the amino acid sequence of ZF5-VPR comprising the amino acid sequence of

(SEQ ID NO: 301)
MAPKKKRKVGIHGVPAAGSSGSLEPGEKPYKCPECGKSFSTSGNLTEHQR
THTGEKPYKCPECGKSFSDSGNLRVHQRTHTGEKPYKCPECGKSFSHKNA
LQNHQRTHTGEKPYKCPECGKSFSRNDTLTEHQRTHTGEKPYKCPECGKS
FSQRAHLERHQRTHTGEKPYKCPECGKSFSRSDKLVRHQRTHTGEKPYKC
PECGKSFSDPGHLVRHQRTHTGEKPTGKKTSASGSGGGSGGDALDDFDLD
MLGSDALDDFDLDMLGSDALDDFDLDMLGSDALDDFDLDMLSGGPKKKRK
VGSQYLPDTDDRHRIEEKRKRTYETFKSIMKKSPFSGPTDPRPPPRRIAV
PSRSSASVPKPAPQPYPFTSSLSTINYDEFPTMVFPSGQISQASALAPAP
PQVLPQAPAPAPAPAMVSALAQAPAPVPVLAPGPPQAVAPPAPKPTQAGE
GTLSEALLQLQFDDEDLGALLGNSTDPAVFTDLASVDNSEFQQLLNQGIP
VAPHTTEPMLMEYPEAITRLVTGAQRPPDPAPAPLGAPGLPNGLLSGDED
FSSIADMDFSALLGSGSGSRDSREGMFLPKPEAGSAISDVFEGREVCQPK
RLRPFHPPGSPWANRPLPASLAPTPTGPVHEPVGSLTPAPVPQPLDPAPA
VTPEASHLLEDPDEETSQAVKALREMADTVIPQKEEAAICGQMDLSHPPP
RGHLDELTTTLESMTEDLNLDSPLTPELNEILDTFLNDECLLHAMHISTG
LSIFDTSLFSGGKRPAATKKAGQAKKKKGSYPYDVPDYA.

In another aspect, the present invention provides a site-specific HNF4α disrupting agent, comprising a polynucleotide encoding the amino acid sequence of ZF5.3-VPR comprising the amino acid sequence of

(SEQ ID NO: 301)
MAPKKKRKVGIHGVPAAGSSGSLEPGEKPYKCPECGKSFSTSGNLTEHQR
THTGEKPYKCPECGKSFSDSGNLRVHQRTHTGEKPYKCPECGKSFSHKNA
LQNHQRTHTGEKPYKCPECGKSFSRNDTLTEHQRTHTGEKPYKCPECGKS
FSQRAHLERHQRTHTGEKPYKCPECGKSFSRSDKLVRHQRTHTGEKPYKC
PECGKSFSDPGHLVRHQRTHTGEKPTGKKTSASGSGGGSGGDALDDFDLD
MLGSDALDDFDLDMLGSDALDDFDLDMLGSDALDDFDLDMLSGGPKKKRK
VGSQYLPDTDDRHRIEEKRKRTYETFKSIMKKSPFSGPTDPRPPPRRIAV
PSRSSASVPKPAPQPYPFTSSLSTINYDEFPTMVFPSGQISQASALAPAP
PQVLPQAPAPAPAPAMVSALAQAPAPVPVLAPGPPQAVAPPAPKPTQAGE
GTLSEALLQLQFDDEDLGALLGNSTDPAVFTDLASVDNSEFQQLLNQGIP
VAPHTTEPMLMEYPEAITRLVTGAQRPPDPAPAPLGAPGLPNGLLSGDED
FSSIADMDFSALLGSGSGSRDSREGMFLPKPEAGSAISDVFEGREVCQPK
RLRPFHPPGSPWANRPLPASLAPTPTGPVHEPVGSLTPAPVPQPLDPAPA
VTPEASHLLEDPDEETSQAVKALREMADTVIPQKEEAAICGQMDLSHPPP
RGHLDELTTTLESMTEDLNLDSPLTPELNEILDTFLNDECLLHAMHISTG
LSIFDTSLFSGGKRPAATKKAGQAKKKKGSYPYDVPDYA.

In yet another aspect, the present invention provides a site-specific HNF4α disrupting agent, comprising a polynucleotide encoding the amino acid sequence of ZF7-VPR comprising the amino acid sequence of

(SEQ ID NO: 302)
MAPKKKRKVGIHGVPAAGSSGSLEPGEKPYKCPECGKSFSRSDHLTNHQR
THTGEKPYKCPECGKSFSTSGSLVRHQRTHTGEKPYKCPECGKSFSQAGH
LASHQRTHTGEKPYKCPECGKSFSRSDKLTEHQRTHTGEKPYKCPECGKS
FSTSGNLTEHQRTHTGEKPYKCPECGKSFSQSSNLVRHQRTHTGEKPYKC
PECGKSFSTHLDLIRHQRTHTGEKPTGKKTSASGSGGGSGGDALDDFDLD
MLGSDALDDFDLDMLGSDALDDFDLDMLGSDALDDFDLDMLSGGPKKKRK
VGSQYLPDTDDRHRIEEKRKRTYETFKSIMKKSPFSGPTDPRPPPRRIAV
PSRSSASVPKPAPQPYPFTSSLSTINYDEFPTMVFPSGQISQASALAPAP
PQVLPQAPAPAPAPAMVSALAQAPAPVPVLAPGPPQAVAPPAPKPTQAGE
GTLSEALLQLQFDDEDLGALLGNSTDPAVFTDLASVDNSEFQQLLNQGIP
VAPHTTEPMLMEYPEAITRLVTGAQRPPDPAPAPLGAPGLPNGLLSGDED
FSSIADMDFSALLGSGSGSRDSREGMFLPKPEAGSAISDVFEGREVCQPK
RLRPFHPPGSPWANRPLPASLAPTPTGPVHEPVGSLTPAPVPQPLDPAPA
VTPEASHLLEDPDEETSQAVKALREMADTVIPQKEEAAICGQMDLSHPPP
RGHLDELTTTLESMTEDLNLDSPLTPELNEILDTFLNDECLLHAMHISTG
LSIFDTSLFSGGKRPAATKKAGQAKKKKGSYPYDVPDYA.

In one aspect, the present invention provides a site-specific HNF4α disrupting agent, comprising a polynucleotide encoding the amino acid sequence of ZF10-VPR comprising the amino acid sequence of

(SEQ ID NO: 303)
MAPKKKRKVGIHGVPAAGSSGSLEPGEKPYKCPECGKSFSQNSTLTEHQR
THTGEKPYKCPECGKSFSERSHLREHQRTHTGEKPYKCPECGKSFSSKKH
LAEHQRTHTGEKPYKCPECGKSFSRNDTLTEHQRTHTGEKPYKCPECGKS
FSDCRDLARHQRTHTGEKPYKCPECGKSFSQSGDLRRHQRTHTGEKPYKC
PECGKSFSTKNSLTEHQRTHTGEKPTGKKTSASGSGGGSGGDALDDFDLD
MLGSDALDDFDLDMLGSDALDDFDLDMLGSDALDDFDLDMLSGGPKKKRK
VGSQYLPDTDDRHRIEEKRKRTYETFKSIMKKSPFSGPTDPRPPPRRIAV
PSRSSASVPKPAPQPYPFTSSLSTINYDEFPTMVFPSGQISQASALAPAP
PQVLPQAPAPAPAPAMVSALAQAPAPVPVLAPGPPQAVAPPAPKPTQAGE
GTLSEALLQLQFDDEDLGALLGNSTDPAVFTDLASVDNSEFQQLLNQGIP
VAPHTTEPMLMEYPEAITRLVTGAQRPPDPAPAPLGAPGLPNGLLSGDED
FSSIADMDFSALLGSGSGSRDSREGMFLPKPEAGSAISDVFEGREVCQPK
RLRPFHPPGSPWANRPLPASLAPTPTGPVHEPVGSLTPAPVPQPLDPAPA
VTPEASHLLEDPDEETSQAVKALREMADTVIPQKEEAAICGQMDLSHPPP
RGHLDELTTTLESMTEDLNLDSPLTPELNEILDTFLNDECLLHAMHISTG
LSIFDTSLFSGGKRPAATKKAGQAKKKKGSYPYDVPDYA.

In another aspect, the present invention provides a site-specific HNF4α disrupting agent, comprising a polynucleotide encoding the amino acid sequence of ZF14-VPR comprising the amino acid sequence of

(SEQ ID NO: 304)
MAPKKKRKVGIHGVPAAGSSGSLEPGEKPYKCPECGKSFSTSGHLVRHQR
THTGEKPYKCPECGKSFSTTGNLTVHQRTHTGEKPYKCPECGKSFSTSGS
LVRHQRTHTGEKPYKCPECGKSFSRSDKLVRHQRTHTGEKPYKCPECGKS
FSRSDELVRHQRTHTGEKPYKCPECGKSFSRSDKLVRHQRTHTGEKPYKC
PECGKSFSQRAHLERHQRTHTGEKPTGKKTSASGSGGGSGGDALDDFDLD
MLGSDALDDFDLDMLGSDALDDFDLDMLGSDALDDFDLDMLSGGPKKKRK
VGSQYLPDTDDRHRIEEKRKRTYETFKSIMKKSPFSGPTDPRPPPRRIAV
PSRSSASVPKPAPQPYPFTSSLSTINYDEFPTMVFPSGQISQASALAPAP
PQVLPQAPAPAPAPAMVSALAQAPAPVPVLAPGPPQAVAPPAPKPTQAGE
GTLSEALLQLQFDDEDLGALLGNSTDPAVFTDLASVDNSEFQQLLNQGIP
VAPHTTEPMLMEYPEAITRLVTGAQRPPDPAPAPLGAPGLPNGLLSGDED
FSSIADMDFSALLGSGSGSRDSREGMFLPKPEAGSAISDVFEGREVCQPK
RLRPFHPPGSPWANRPLPASLAPTPTGPVHEPVGSLTPAPVPQPLDPAPA
VTPEASHLLEDPDEETSQAVKALREMADTVIPQKEEAAICGQMDLSHPPP
RGHLDELTTTLESMTEDLNLDSPLTPELNEILDTFLNDECLLHAMHISTG
LSIFDTSLFSGGKRPAATKKAGQAKKKKGSYPYDVPDYA.

In one aspect, the present invention provides a site-specific HNF4α disrupting agent, comprising a polynucleotide encoding the amino acid sequence of ZF15-VPR comprising the amino acid sequence of

(SEQ ID NO: 305)
MAPKKKRKVGIHGVPAAGSSGSLEPGEKPYKCPECGKSFSRNDTLTEHQR
THTGEKPYKCPECGKSFSRNDALTEHQRTHTGEKPYKCPECGKSFSTSGE
LVRHQRTHTGEKPYKCPECGKSFSRSDNLVRHQRTHTGEKPYKCPECGKS
FSRSDELVRHQRTHTGEKPYKCPECGKSFSRSDKLVRHQRTHTGEKPYKC
PECGKSFSQSSNLVRHQRTHTGEKPTGKKTSASGSGGGSGGDALDDFDLD
MLGSDALDDFDLDMLGSDALDDFDLDMLGSDALDDFDLDMLSGGPKKKRK
VGSQYLPDTDDRHRIEEKRKRTYETFKSIMKKSPFSGPTDPRPPPRRIAV
PSRSSASVPKPAPQPYPFTSSLSTINYDEFPTMVFPSGQISQASALAPAP
PQVLPQAPAPAPAPAMVSALAQAPAPVPVLAPGPPQAVAPPAPKPTQAGE
GTLSEALLQLQFDDEDLGALLGNSTDPAVFTDLASVDNSEFQQLLNQGIP
VAPHTTEPMLMEYPEAITRLVTGAQRPPDPAPAPLGAPGLPNGLLSGDED
FSSIADMDFSALLGSGSGSRDSREGMFLPKPEAGSAISDVFEGREVCQPK
RLRPFHPPGSPWANRPLPASLAPTPTGPVHEPVGSLTPAPVPQPLDPAPA
VTPEASHLLEDPDEETSQAVKALREMADTVIPQKEEAAICGQMDLSHPPP
RGHLDELTTTLESMTEDLNLDSPLTPELNEILDTFLNDECLLHAMHISTG
LSIFDTSLFSGGKRPAATKKAGQAKKKKGSYPYDVPDYA.

In one aspect, the present invention provides a bicistronic site-specific HNF4α disrupting agent, comprising a polynucleotide encoding the amino acid sequence of a bicistronic ZF5.3-VPR-tPT2a-ZF7-VPR comprising the amino acid sequence of

(SEQ ID NO: 306)
MAPKKKRKVGIHGVPAAGSSGSSGSLEPGEKPYKCPECGKSFSTSGNLTE
HQRTHTGEKPYKCPECGKSFSDSGNLRVHQRTHTGEKPYKCPECGKSFSH
KNALQNHQRTHTGEKPYKCPECGKSFSRNDTLTEHQRTHTGEKPYKCPEC
GKSFSQRAHLERHQRTHTGEKPYKCPECGKSFSRSDKLVRHQRTHTGEKP
YKCPECGKSFSDPGHLVRHQRTHTGEKPTGKKTSASGSGGGSGGDALDDF
DLDMLGSDALDDFDLDMLGSDALDDFDLDMLGSDALDDFDLDMLSGGPKK
KRKVGSQYLPDTDDRHRIEEKRKRTYETFKSIMKKSPFSGPTDPRPPPRR
IAVPSRSSASVPKPAPQPYPFTSSLSTINYDEFPTMVFPSGQISQASALA
PAPPQVLPQAPAPAPAPAMVSALAQAPAPVPVLAPGPPQAVAPPAPKPTQ
AGEGTLSEALLQLQFDDEDLGALLGNSTDPAVFTDLASVDNSEFQQLLNQ
GIPVAPHTTEPMLMEYPEAITRLVTGAQRPPDPAPAPLGAPGLPNGLLSG
DEDFSSIADMDFSALLGSGSGSRDSREGMFLPKPEAGSAISDVFEGREVC
QPKRLRPFHPPGSPWANRPLPASLAPTPTGPVHEPVGSLTPAPVPQPLDP
APAVTPEASHLLEDPDEETSQAVKALREMADTVIPQKEEAAICGQMDLSH
PPPRGHLDELTTTLESMTEDLNLDSPLTPELNEILDTFLNDECLLHAMHI
STGLSIFDTSLFSGGKRPAATKKAGQAKKKKGSYPYDVPDYAATNFSLLK
QAGDVEENPGPTSAGKLGSGEGRGSLLTCGDVEENPGPLEGSSGSGSLEP
GEKPYKCPECGKSFSRSDHLTNHQRTHTGEKPYKCPECGKSFSTSGSLVR
HQRTHTGEKPYKCPECGKSFSQAGHLASHQRTHTGEKPYKCPECGKSFSR
SDKLTEHQRTHTGEKPYKCPECGKSFSTSGNLTEHQRTHTGEKPYKCPEC
GKSFSQSSNLVRHQRTHTGEKPYKCPECGKSFSTHLDLIRHQRTHTGEKP
TGKKTSASGSGGGSGGDALDDFDLDMLGSDALDDFDLDMLGSDALDDFDL
DMLGSDALDDFDLDMLSGGPKKKRKVGSQYLPDTDDRHRIEEKRKRTYET
FKSIMKKSPFSGPTDPRPPPRRIAVPSRSSASVPKPAPQPYPFTSSLSTI
NYDEFPTMVFPSGQISQASALAPAPPQVLPQAPAPAPAPAMVSALAQAPA
PVPVLAPGPPQAVAPPAPKPTQAGEGTLSEALLQLQFDDEDLGALLGNST
DPAVFTDLASVDNSEFQQLLNQGIPVAPHTTEPMLMEYPEAITRLVTGAQ
RPPDPAPAPLGAPGLPNGLLSGDEDFSSIADMDFSALLGSGSGSRDSREG
MFLPKPEAGSAISDVFEGREVCQPKRLRPFHPPGSPWANRPLPASLAPTP
TGPVHEPVGSLTPAPVPQPLDPAPAVTPEASHLLEDPDEETSQAVKALRE
MADTVIPQKEEAAICGQMDLSHPPPRGHLDELTTTLESMTEDLNLDSPLT
PELNEILDTFLNDECLLHAMHISTGLSIFDTSLFSGGKRPAATKKAGQAK
KKKGSYPYDVPDYA.

In one aspect, the present invention provides a bicistronic site-specific HNF4α disrupting agent, comprising a polynucleotide encoding the amino acid sequence of a bicistronic ZF7-VPR-tPT2a-ZF5.3-VPR comprising the amino acid sequence of

(SEQ ID NO: 178)
MAPKKKRKVGIHGVPAAGSSGSLEPGEKPYKCPECGKSFSRSDHLTNHQR
THTGEKPYKCPECGKSFSTSGSLVRHQRTHTGEKPYKCPECGKSFSQAGH
LASHQRTHTGEKPYKCPECGKSFSRSDKLTEHQRTHTGEKPYKCPECGKS
FSTSGNLTEHQRTHTGEKPYKCPECGKSFSQSSNLVRHQRTHTGEKPYKC
PECGKSFSTHLDLIRHQRTHTGEKPTGKKTSASGSGGGSGGDALDDFDLD
MLGSDALDDFDLDMLGSDALDDFDLDMLGSDALDDFDLDMLSGGPKKKRK
VGSQYLPDTDDRHRIEEKRKRTYETFKSIMKKSPFSGPTDPRPPPRRIAV
PSRSSASVPKPAPQPYPFTSSLSTINYDEFPTMVFPSGQISQASALAPAP
PQVLPQAPAPAPAPAMVSALAQAPAPVPVLAPGPPQAVAPPAPKPTQAGE
GTLSEALLQLQFDDEDLGALLGNSTDPAVFTDLASVDNSEFQQLLNQGIP
VAPHTTEPMLMEYPEAITRLVTGAQRPPDPAPAPLGAPGLPNGLLSGDED
FSSIADMDFSALLGSGSGSRDSREGMFLPKPEAGSAISDVFEGREVCQPK
RLRPFHPPGSPWANRPLPASLAPTPTGPVHEPVGSLTPAPVPQPLDPAPA
VTPEASHLLEDPDEETSQAVKALREMADTVIPQKEEAAICGQMDLSHPPP
RGHLDELTTTLESMTEDLNLDSPLTPELNEILDTFLNDECLLHAMHISTG
LSIFDTSLFSGGKRPAATKKAGQAKKKKGSYPYDVPDYAATNFSLLKQAG
DVEENPGPTSAGKLGSGEGRGSLLTCGDVEENPGPLEGSSGSLEPGEKPY
KCPECGKSFSTSGNLTEHQRTHTGEKPYKCPECGKSFSDSGNLRVHQRTH
TGEKPYKCPECGKSFSHKNALQNHQRTHTGEKPYKCPECGKSFSRNDTLT
EHQRTHTGEKPYKCPECGKSFSQRAHLERHQRTHTGEKPYKCPECGKSFS
RSDKLVRHQRTHTGEKPYKCPECGKSFSDPGHLVRHQRTHTGEKPTGKKT
SASGSGGGSGGDALDDFDLDMLGSDALDDFDLDMLGSDALDDFDLDMLGS
DALDDFDLDMLSGGPKKKRKVGSQYLPDTDDRHRIEEKRKRTYETFKSIM
KKSPFSGPTDPRPPPRRIAVPSRSSASVPKPAPQPYPFTSSLSTINYDEF
PTMVFPSGQISQASALAPAPPQVLPQAPAPAPAPAMVSALAQAPAPVPVL
APGPPQAVAPPAPKPTQAGEGTLSEALLQLQFDDEDLGALLGNSTDPAVF
TDLASVDNSEFQQLLNQGIPVAPHTTEPMLMEYPEAITRLVTGAQRPPDP
APAPLGAPGLPNGLLSGDEDFSSIADMDFSALLGSGSGSRDSREGMFLPK
PEAGSAISDVFEGREVCQPKRLRPFHPPGSPWANRPLPASLAPTPTGPVH
EPVGSLTPAPVPQPLDPAPAVTPEASHLLEDPDEETSQAVKALREMADTV
IPQKEEAAICGQMDLSHPPPRGHLDELTTTLESMTEDLNLDSPLTPELNE
ILDTFLNDECLLHAMHISTGLSIFDTSLFSGGKRPAATKKAGQAKKKKGS
YPYDVPDYA.

In one aspect, the present invention provides a bicistronic site-specific HNF4α disrupting agent, comprising a polynucleotide encoding the amino acid sequence of a bicistronic ZF5.3-VPR-tPT2a-ZF7-p300 comprising the amino acid sequence of

(SEQ ID NO: 307)
MAPKKKRKVGIHGVPAAGSSGSSGSLEPGEKPYKCPECGKSFSTSGNLTE
HQRTHTGEKPYKCPECGKSFSDSGNLRVHQRTHTGEKPYKCPECGKSFSH
KNALQNHQRTHTGEKPYKCPECGKSFSRNDTLTEHQRTHTGEKPYKCPEC
GKSFSQRAHLERHQRTHTGEKPYKCPECGKSFSRSDKLVRHQRTHTGEKP
YKCPECGKSFSDPGHLVRHQRTHTGEKPTGKKTSASGSGGGSGGDALDDF
DLDMLGSDALDDFDLDMLGSDALDDFDLDMLGSDALDDFDLDMLSGGPKK
KRKVGSQYLPDTDDRHRIEEKRKRTYETFKSIMKKSPFSGPTDPRPPPRR
IAVPSRSSASVPKPAPQPYPFTSSLSTINYDEFPTMVFPSGQISQASALA
PAPPQVLPQAPAPAPAPAMVSALAQAPAPVPVLAPGPPQAVAfPPAPKPT
QAGEGTLSEALLQLQFDDEDLGALLGNSTDPAVFTDLASVDNSEFQQLLN
QGIPVAPHTTEPMLMEYPEAITRLVTGAQRPPDPAPAPLGAPGLPNGLLS
GDEDFSSIADMDFSALLGSGSGSRDSREGMFLPKPEAGSAISDVFEGREV
CQPKRLRPFHPPGSPWANRPLPASLAPTPTGPVHEPVGSLTPAPVPQPLD
PAPAVTPEASHLLEDPDEETSQAVKALREMADTVIPQKEEAAICGQMDLS
HPPPRGHLDELTTTLESMTEDLNLDSPLTPELNEILDTFLNDECLLHAMH
ISTGLSIFDTSLFSGGKRPAATKKAGQAKKKKGSYPYDVPDYAATNFSLL
KQAGDVEENPGPTSAGKLGSGEGRGSLLTCGDVEENPGPLEGSSGSGSLE
PGEKPYKCPECGKSFSRSDHLTNHQRTHTGEKPYKCPECGKSFSTSGSLV
RHQRTHTGEKPYKCPECGKSFSQAGHLASHQRTHTGEKPYKCPECGKSFS
RSDKLTEHQRTHTGEKPYKCPECGKSFSTSGNLTEHQRTHTGEKPYKCPE
CGKSFSQSSNLVRHQRTHTGEKPYKCPECGKSFSTHLDLIRHQRTHTGEK
PTGKKTSASGSGGGSGGIFKPEELRQALMPTLEALYRQDPESLPFRQPVD
PQLLGIPDYFDIVKSPMDLSTIKRKLDTGQYQEPWQYVDDIWLMFNNAWL
YNRKTSRVYKYCSKLSEVFEQEIDPVMQSLGYCCGRKLEFSPQTLCCYGK
QLCTIPRDATYYSYQNRYHFCEKCFNEIQGESVSLGDDPSQPQTTINKEQ
FSKRKNDTLDPELFVECTECGRKMHQICVLHHEIIWPAGFVCDGCLKKSA
RTRKENKFSAKRLPSTRLGTFLENRVNDFLRRQNHPESGEVTVRVVHASD
KTVEVKPGMKARFVDSGEMAESFPYRTKALFAFEEIDGVDLCFFGMHVQE
YGSDCPPPNQRRVYISYLDSVHFFRPKCLRTAVYHEILIGYLEYVKKLGY
TTGHIWACPPSEGDDYIFHCHPPDQKIPKPKRLQEWYKKMLDKAVSERIV
HDYKDIFKQATEDRLTSAKELPYFEGDFWPNVLEESIKELEQEEEERKRE
ENTSNESTDVTKGDSKNAKKKNNKKTSKNKSSLSRGNKKKPGMPNVSNDL
SQKLYATMEKHKEVFFVIRLIAGPAANSLPPIVDPDPLIPCDLMDGRDAF
LTLARDKHLEFSSLRRAQWSTMCMLVELHTQSQDSGGKRPAATKKAGQAK
KKKGSYPYDVPDYA.

In one aspect, the present invention provides a bicistronic site-specific HNF4α disrupting agent, comprising a polynucleotide encoding the amino acid sequence of a bicistronic ZF7-p300-tPT2a-ZF5.3-VPR comprising the amino acid sequence of

(SEQ ID NO: 308)
MAPKKKRKVGIHGVPAAGSSGSLEPGEKPYKCPECGKSFSRSDHLTNHQR
THTGEKPYKCPECGKSFSTSGSLVRHQRTHTGEKPYKCPECGKSFSQAGH
LASHQRTHTGEKPYKCPECGKSFSRSDKLTEHQRTHTGEKPYKCPECGKS
FSTSGNLTEHQRTHTGEKPYKCPECGKSFSQSSNLVRHQRTHTGEKPYKC
PECGKSFSTHLDLIRHQRTHTGEKPTGKKTSASGSGGGSGGIFKPEELRQ
ALMPTLEALYRQDPESLPFRQPVDPQLLGIPDYFDIVKSPMDLSTIKRKL
DTGQYQEPWQYVDDIWLMFNNAWLYNRKTSRVYKYCSKLSEVFEQEIDPV
MQSLGYCCGRKLEFSPQTLCCYGKQLCTIPRDATYYSYQNRYHFCEKCFN
EIQGESVSLGDDPSQPQTTINKEQFSKRKNDTLDPELFVECTECGRKMHQ
ICVLHHEIIWPAGFVCDGCLKKSARTRKENKFSAKRLPSTRLGTFLENRV
NDFLRRQNHPESGEVTVRVVHASDKTVEVKPGMKARFVDSGEMAESFPYR
TKALFAFEEIDGVDLCFFGMHVQEYGSDCPPPNQRRVYISYLDSVHFFRP
KCLRTAVYHEILIGYLEYVKKLGYTTGHIWACPPSEGDDYIFHCHPPDQK
IPKPKRLQEWYKKMLDKAVSERIVHDYKDIFKQATEDRLTSAKELPYFEG
DFWPNVLEESIKELEQEEEERKREENTSNESTDVTKGDSKNAKKKNNKKT
SKNKSSLSRGNKKKPGMPNVSNDLSQKLYATMEKHKEVFFVIRLIAGPAA
NSLPPIVDPDPLIPCDLMDGRDAFLTLARDKHLEFSSLRRAQWSTMCMLV
ELHTQSQDSGGKRPAATKKAGQAKKKKGSYPYDVPDYAATNFSLLKQAGD
VEENPGPTSAGKLGSGEGRGSLLTCGDVEENPGPLEGSSGSLEPGEKPYK
CPECGKSFSTSGNLTEHQRTHTGEKPYKCPECGKSFSDSGNLRVHQRTHT
GEKPYKCPECGKSFSHKNALQNHQRTHTGEKPYKCPECGKSFSRNDTLTE
HQRTHTGEKPYKCPECGKSFSQRAHLERHQRTHTGEKPYKCPECGKSFSR
SDKLVRHQRTHTGEKPYKCPECGKSFSDPGHLVRHQRTHTGEKPTGKKTS
ASGSGGGSGGDALDDFDLDMLGSDALDDFDLDMLGSDALDDFDLDMLGSD
ALDDFDLDMLSGGPKKKRKVGSQYLPDTDDRHRIEEKRKRTYETFKSIMK
KSPFSGPTDPRPPPRRIAVPSRSSASVPKPAPQPYPFTSSLSTINYDEFP
TMVFPSGQISQASALAPAPPQVLPQAPAPAPAPAMVSALAQAPAPVPVLA
PGPPQAVAPPAPKPTQAGEGTLSEALLQLQFDDEDLGALLGNSTDPAVFT
DLASVDNSEFQQLLNQGIPVAPHTTEPMLMEYPEAITRLVTGAQRPPDPA
PAPLGAPGLPNGLLSGDEDFSSIADMDFSALLGSGSGSRDSREGMFLPKP
EAGSAISDVFEGREVCQPKRLRPFHPPGSPWANRPLPASLAPTPTGPVHE
PVGSLTPAPVPQPLDPAPAVTPEASHLLEDPDEETSQAVKALREMADTVI
PQKEEAAICGQMDLSHPPPRGHLDELTTTLESMTEDLNLDSPLTPELNEI
LDTFLNDECLLHAMHISTGLSIFDTSLFSGGKRPAATKKAGQAKKKKGSY
PYDVPDYA.

The present invention also provides vectors, such as viral expression vectors and cells comprising the site-specific HNF4α disrupting agents of the invention as well as the vectors of the invention.

In some embodiments, the site-specific HNF4α disrupting agents of the invention are present in a composition, such as a pharmaceutical composition.

In some embodiments, the pharmaceutical composition comprises a lipid formulation.

In some embodiments, the lipid formulation comprises one or more cationic lipids, one or more non-cationic lipids, one or more cholesterol-based lipids, or one or more PEG-modified lipids, or combinations of any of the foregoing.

In some embodiments, the pharmaceutical composition comprises a lipid nanoparticle.

In one aspect, the present invention provides a pharmaceutical composition comprising a site-specific HNF4α disrupting agent, comprising a polynucleotide encoding the amino acid sequence of ZF5.3-VPR comprising the amino acid sequence of

(SEQ ID NO: 301)
MAPKKKRKVGIHGVPAAGSSGSLEPGEKPYKCPECGKSFSTSGNLTEHQR
THTGEKPYKCPECGKSFSDSGNLRVHQRTHTGEKPYKCPECGKSFSHKNA
LQNHQRTHTGEKPYKCPECGKSFSRNDTLTEHQRTHTGEKPYKCPECGKS
FSQRAHLERHQRTHTGEKPYKCPECGKSFSRSDKLVRHQRTHTGEKPYKC
PECGKSFSDPGHLVRHQRTHTGEKPTGKKTSASGSGGGSGGDALDDFDLD
MLGSDALDDFDLDMLGSDALDDFDLDMLGSDALDDFDLDMLSGGPKKKRK
VGSQYLPDTDDRHRIEEKRKRTYETFKSIMKKSPFSGPTDPRPPPRRIAV
PSRSSASVPKPAPQPYPFTSSLSTINYDEFPTMVFPSGQISQASALAPAP
PQVLPQAPAPAPAPAMVSALAQAPAPVPVLAPGPPQAVAPPAPKPTQAGE
GTLSEALLQLQFDDEDLGALLGNSTDPAVFTDLASVDNSEFQQLLNQGIP
VAPHTTEPMLMEYPEAITRLVTGAQRPPDPAPAPLGAPGLPNGLLSGDED
FSSIADMDFSALLGSGSGSRDSREGMFLPKPEAGSAISDVFEGREVCQPK
RLRPFHPPGSPWANRPLPASLAPTPTGPVHEPVGSLTPAPVPQPLDPAPA
VTPEASHLLEDPDEETSQAVKALREMADTVIPQKEEAAICGQMDLSHPPP
RGHLDELTTTLESMTEDLNLDSPLTPELNEILDTFLNDECLLHAMHISTG
LSIFDTSLFSGGKRPAATKKAGQAKKKKGSYPYDVPDYA,

and a lipid nanoparticle.

In one aspect, the present invention provides a pharmaceutical composition comprising a site-specific HNF4α disrupting agent, comprising a polynucleotide encoding the amino acid sequence of ZF5.3-VPR comprising the amino acid sequence of

(SEQ ID NO: 301)
MAPKKKRKVGIHGVPAAGSSGSLEPGEKPYKCPECGKSFSTSGNLTEHQR
THTGEKPYKCPECGKSFSDSGNLRVHQRTHTGEKPYKCPECGKSFSHKNA
LQNHQRTHTGEKPYKCPECGKSFSRNDTLTEHQRTHTGEKPYKCPECGKS
FSQRAHLERHQRTHTGEKPYKCPECGKSFSRSDKLVRHQRTHTGEKPYKC
PECGKSFSDPGHLVRHQRTHTGEKPTGKKTSASGSGGGSGGDALDDFDLD
MLGSDALDDFDLDMLGSDALDDFDLDMLGSDALDDFDLDMLSGGPKKKRK
VGSQYLPDTDDRHRIEEKRKRTYETFKSIMKKSPFSGPTDPRPPPRRIAV
PSRSSASVPKPAPQPYPFTSSLSTINYDEFPTMVFPSGQISQASALAPAP
PQVLPQAPAPAPAPAMVSALAQAPAPVPVLAPGPPQAVAPPAPKPTQAGE
GTLSEALLQLQFDDEDLGALLGNSTDPAVFTDLASVDNSEFQQLLNQGIP
VAPHTTEPMLMEYPEAITRLVTGAQRPPDPAPAPLGAPGLPNGLLSGDED
FSSIADMDFSALLGSGSGSRDSREGMFLPKPEAGSAISDVFEGREVCQPK
RLRPFHPPGSPWANRPLPASLAPTPTGPVHEPVGSLTPAPVPQPLDPAPA
VTPEASHLLEDPDEETSQAVKALREMADTVIPQKEEAAICGQMDLSHPPP
RGHLDELTTTLESMTEDLNLDSPLTPELNEILDTFLNDECLLHAMHISTG
LSIFDTSLFSGGKRPAATKKAGQAKKKKGSYPYDVPDYA;

and

a site-specific HNF4α disrupting agent, comprising a polynucleotide encoding the amino acid sequence of ZF7-VPR comprising the amino acid sequence of

(SEQ ID NO: 302)
MAPKKKRKVGIHGVPAAGSSGSLEPGEKPYKCPECGKSFSRSDHLTNHQR
THTGEKPYKCPECGKSFSTSGSLVRHQRTHTGEKPYKCPECGKSFSQAGH
LASHQRTHTGEKPYKCPECGKSFSRSDKLTEHQRTHTGEKPYKCPECGKS
FSTSGNLTEHQRTHTGEKPYKCPECGKSFSQSSNLVRHQRTHTGEKPYKC
PECGKSFSTHLDLIRHQRTHTGEKPTGKKTSASGSGGGSGGDALDDFDLD
MLGSDALDDFDLDMLGSDALDDFDLDMLGSDALDDFDLDMLSGGPKKKRK
VGSQYLPDTDDRHRIEEKRKRTYETFKSIMKKSPFSGPTDPRPPPRRIAV
PSRSSASVPKPAPQPYPFTSSLSTINYDEFPTMVFPSGQISQASALAPAP
PQVLPQAPAPAPAPAMVSALAQAPAPVPVLAPGPPQAVAPPAPKPTQAGE
GTLSEALLQLQFDDEDLGALLGNSTDPAVFTDLASVDNSEFQQLLNQGIP
VAPHTTEPMLMEYPEAITRLVTGAQRPPDPAPAPLGAPGLPNGLLSGDED
FSSIADMDFSALLGSGSGSRDSREGMFLPKPEAGSAISDVFEGREVCQPK
RLRPFHPPGSPWANRPLPASLAPTPTGPVHEPVGSLTPAPVPQPLDPAPA
VTPEASHLLEDPDEETSQAVKALREMADTVIPQKEEAAICGQMDLSHPPP
RGHLDELTTTLESMTEDLNLDSPLTPELNEILDTFLNDECLLHAMHISTG
LSIFDTSLFSGGKRPAATKKAGQAKKKKGSYPYDVPDYA;

and a lipid nanoparticle.

In one aspect, the present invention provides a pharmaceutical composition, comprising a site-specific HNF4α disrupting agent, comprising a polynucleotide encoding the amino acid sequence of a bicistronic ZF5.3-VPR-tPT2a-ZF7-VPR comprising the amino acid sequence of

(SEQ ID NO: 306)
MAPKKKRKVGIHGVPAAGSSGSSGSLEPGEKPYKCPECGKSFSTSGNLTE
HQRTHTGEKPYKCPECGKSFSDSGNLRVHQRTHTGEKPYKCPECGKSFSH
KNALQNHQRTHTGEKPYKCPECGKSFSRNDTLTEHQRTHTGEKPYKCPEC
GKSFSQRAHLERHQRTHTGEKPYKCPECGKSFSRSDKLVRHQRTHTGEKP
YKCPECGKSFSDPGHLVRHQRTHTGEKPTGKKTSASGSGGGSGGDALDDF
DLDMLGSDALDDFDLDMLGSDALDDFDLDMLGSDALDDFDLDMLSGGPKK
KRKVGSQYLPDTDDRHRIEEKRKRTYETFKSIMKKSPFSGPTDPRPPPRR
IAVPSRSSASVPKPAPQPYPFTSSLSTINYDEFPTMVFPSGQISQASALA
PAPPQVLPQAPAPAPAPAMVSALAQAPAPVPVLAPGPPQAVAPPAPKPTQ
AGEGTLSEALLQLQFDDEDLGALLGNSTDPAVFTDLASVDNSEFQQLLNQ
GIPVAPHTTEPMLMEYPEAITRLVTGAQRPPDPAPAPLGAPGLPNGLLSG
DEDFSSIADMDFSALLGSGSGSRDSREGMFLPKPEAGSAISDVFEGREVC
QPKRLRPFHPPGSPWANRPLPASLAPTPTGPVHEPVGSLTPAPVPQPLDP
APAVTPEASHLLEDPDEETSQAVKALREMADTVIPQKEEAAICGQMDLSH
PPPRGHLDELTTTLESMTEDLNLDSPLTPELNEILDTFLNDECLLHAMHI
STGLSIFDTSLFSGGKRPAATKKAGQAKKKKGSYPYDVPDYAATNFSLLK
QAGDVEENPGPTSAGKLGSGEGRGSLLTCGDVEENPGPLEGSSGSGSLEP
GEKPYKCPECGKSFSRSDHLTNHQRTHTGEKPYKCPECGKSFSTSGSLVR
HQRTHTGEKPYKCPECGKSFSQAGHLASHQRTHTGEKPYKCPECGKSFSR
SDKLTEHQRTHTGEKPYKCPECGKSFSTSGNLTEHQRTHTGEKPYKCPEC
GKSFSQSSNLVRHQRTHTGEKPYKCPECGKSFSTHLDLIRHQRTHTGEKP
TGKKTSASGSGGGSGGDALDDFDLDMLGSDALDDFDLDMLGSDALDDFDL
DMLGSDALDDFDLDMLSGGPKKKRKVGSQYLPDTDDRHRIEEKRKRTYET
FKSIMKKSPFSGPTDPRPPPRRIAVPSRSSASVPKPAPQPYPFTSSLSTI
NYDEFPTMVFPSGQISQASALAPAPPQVLPQAPAPAPAPAMVSALAQAPA
PVPVLAPGPPQAVAPPAPKPTQAGEGTLSEALLQLQFDDEDLGALLGNST
DPAVFTDLASVDNSEFQQLLNQGIPVAPHTTEPMLMEYPEAITRLVTGAQ
RPPDPAPAPLGAPGLPNGLLSGDEDFSSIADMDFSALLGSGSGSRDSREG
MFLPKPEAGSAISDVFEGREVCQPKRLRPFHPPGSPWANRPLPASLAPTP
TGPVHEPVGSLTPAPVPQPLDPAPAVTPEASHLLEDPDEETSQAVKALRE
MADTVIPQKEEAAICGQMDLSHPPPRGHLDELTTTLESMTEDLNLDSPLT
PELNEILDTFLNDECLLHAMHISTGLSIFDTSLFSGGKRPAATKKAGQAK
KKKGSYPYDVPDYA;

and a lipid nanoparticle.

In one aspect, the present invention provides a method of modulating expression of hepatocyte nuclear factor 4 alpha-(HNF4α) in a cell. The method includes contacting the cell with a site-specific HNF4α disrupting agent, the disrupting agent comprising a site-specific HNF4α targeting moiety which targets an HNF4α expression control region, and an effector molecule, thereby modulating expression of HNF4α in the cell.

The modulation of expression may be enhanced expression of HNF4α in the cell or reduced expression of HNF4α in the cell.

### BRIEF DESCRIPTION OF THE DRAWINGS

FIG. 1 depicts a chromosomal view of a portion of the upstream region and coding sequence of HNF4α and the positions of guide RNAs and pools of guide RNAs described herein.

FIG. 2 is a graph depicting the percent of HNF4α mRNA remaining in HepG2 cells at 72 hours after contacting the cells with the indicated pools of site-specific HNF4α targeting moieties and an effector molecule comprising dCas and KRAB at the indicated doses.

FIG. 3 is a graph depicting the percent of HNF4α mRNA remaining in HepG2 cells at 72 hours after contacting the cells with either the indicated site-specific HNF4α targeting moieties and an effector molecule comprising dCas and KRAB, or the indicated pools of HNF4α targeting moieties and an effector molecule comprising dCas and KRAB, at the indicated doses. Untreated cells and cells contacted with dCas9 alone were used as controls.

FIG. 4A is a graph depicting the percent of HNF4α mRNA measured in A549 cells at 48 hours after contacting the cells with the indicated pools of HNF4α targeting moieties and an effector molecule comprising dCas and VPR, at the indicated doses. SH is the abbreviation for “Safe Harbor” which is a non-target guide control.

FIG. 4B is a graph depicting the percent of HNF4α mRNA measured in LX-2 cells at 48 hours after contacting the cells with the indicated pools of HNF4α targeting moieties and an effector molecule comprising dCas and VPR at the indicated doses.

FIG. 5 is a graph depicting activation of HNF4α by dCas9-VPR in LX-2 cells with transfection using LNP formulations.

FIG. 6 is a graph depicting activation of HNF4 α by dCas9-VPR in HepG2 cells with transfection using LNP formulations.

FIG. 7 are immunohistological images depicting that HNF4α protein induced by dCas9-VPR Pool 1 is localized to the nucleus.

FIG. 8A and FIG. 8B are schematics depicting the structure of the human HNF4α promoter region and the isoforms of HNF4α.

FIG. 9A and FIG. 9B schematically depict the localization to the HNF4α promoter region of the various site-specific HNF4α targeting moieties. FIG. 9A and FIG. 9B disclose SEQ ID NOS 2526-2527, and 2519-2525, respectively, in order of appearance.

FIG. 10 schematically depicts the structure of an exemplary zinc finger-VPR fusion protein of the invention.

FIG. 11 is a graph depicting activation of HNF4α in LX-2 cells using ZF-VPR mRNAs.

FIG. 12 is a graph depicting activation of HNF4α in LX-2 cells with TAL-VPRs and ZF-VPRs.

FIG. 13 is a graph depicting activation of HNF4α in LX-2 Cells with ATUM-Codon Optimized ZF5-VPR variants.

FIG. 14 is a graph depicting the durability of VPR activation of HNF4α in K562 cells with ZF5-VPR, ZF5-p300, ZF5-VPR and ZF5-p300, and ZF5-VPR and ZF7-VPR.

FIG. 15 is a graph depicting the durability of VPR activation of HNF4α in K562 cells with ZF5-VPR and ZF7-VPR.

FIG. 16 is a graph depicting the durability of VPR activation of HNF4α in K562 cells with ZF5-VPR and ZF7-VPR, and ZF5.3-VPR and ZF7-VPR.

FIG. 17 is a graph depicting the change in expression level of biomarkers downstream of HNF4α following activation of HNF4α by dCas9-VPR in LX-2 cells.

FIG. 18 is a graph depicting the change in expression level of biomarkers of HNF4α following activation of HNF4α by ZF5-VPR, ZF5.3-VPR, ZF5-VPR and ZF7-VPR, and ZF5.3-VPR and ZF7-VPR in LX-2 cells.

FIG. 19 is a graph depicting screening of ZF11, ZF13, and ZF14 in LX-2 cells and synergy in activating HNF4α by ZF5-VPR and ZF7-VPR, and ZF5.3-VPR and ZF7-VPR.

FIG. 20 is a graph depicting activation of HNF4α using dCas9-VPR3-Pool 1 and screening of ZF-VPR combinations FIG. 21 is a graph depicting HNF4α activation in FRG-KO mouse liver humanized hepatocytes (Yecuris human hepatocytes).

FIG. 22 is a graph depicting activation of HNF4α in LX-2 cells with bicistronic ZF5.3-VPR and ZF7-VPR.

FIG. 23 is a graph depicting the effect of repeat dosing of Yecuris hepatocytes with various ZF-VPR and ZF-p300 on HNF4α gene expression.

FIG. 24 is a graph depicting activation of HNF4α in Yecuris hepatocytes with bicistronic ZF-effector constructs.

FIG. 25 is graph depicting the 10 days durability of VPR activation of HNF4α in K562 cells with bicistronic ZF-effector constructs.

### DETAILED DESCRIPTION OF THE INVENTION

The present invention provides agents and compositions for modulating expression (e.g., enhanced or reduced expression) of a hepatocyte nuclear factor 4 alpha (HNF4α) gene by targeting an HNF4α expression control region. The HNF4α gene may be in a cell, e.g., a mammalian cell, such as a mammalian somatic cell, e.g., a human somatic cell. The present invention also provides methods of using the agents and compositions of the invention for modulating the expression of an HNF4α gene or for treating a subject who would benefit from modulating the expression of an HNF4α gene, e.g., a subject suffering or prone to suffering from an HNF4α-associated disease.

The agents of the invention are referenced to herein as site-specific HNF4α disrupting agents and are described in Section II, below.

### I. DEFINITIONS

In order that the present invention may be more readily understood, certain terms are first defined. In addition, it should be noted that whenever a value or range of values of a parameter are recited, it is intended that values and ranges intermediate to the recited values are also intended to be part of this invention.

The articles “a” and “an” are used herein to refer to one or to more than one (i.e., to at least one) of the grammatical object of the article. By way of example, “an element” means one element or more than one element, e.g., a plurality of elements.

The term “including” is used herein to mean, and is used interchangeably with, the phrase “including but not limited to”. The term “or” is used herein to mean, and is used interchangeably with, the term “and/or,” unless context clearly indicates otherwise.

The term “about” is used herein to mean within the typical ranges of tolerances in the art. For example, “about” can be understood as about 2 standard deviations from the mean. In certain embodiments, about means ±10%. In certain embodiments, about means ±5%. When about is present before a series of numbers or a range, it is understood that “about” can modify each of the numbers in the series or range.

The term “at least” prior to a number or series of numbers is understood to include the number adjacent to the term “at least”, and all subsequent numbers or integers that could logically be included, as clear from context. For example, the number of nucleotides in a nucleic acid molecule must be an integer. For example, “at least 18 nucleotides of a 21 nucleotide nucleic acid molecule” means that 18, 19, 20, or 21 nucleotides have the indicated property. When at least is present before a series of numbers or a range, it is understood that “at least” can modify each of the numbers in the series or range.

As used herein, “no more than” or “less than” is understood as the value adjacent to the phrase and logical lower values or integers, as logical from context, to zero. When “no more than” is present before a series of numbers or a range, it is understood that “no more than” can modify each of the numbers in the series or range.

As used herein, the term “substantially” refers to the qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result. The term “substantially” may therefore be used in some embodiments herein to capture potential lack of completeness inherent in many biological and chemical phenomena.

As used herein, the terms “hepatocyte nuclear factor 4 alpha,” “HNF4α,” and “HNF4A,” as used interchangeably herein, refer to the gene as well as the well known encoded protein which is a nuclear transcription factor which binds DNA as a homodimer. The encoded protein controls the expression of several genes, including hepatocyte nuclear factor 1 alpha, a transcription factor which regulates the expression of several hepatic genes. HNF4α also plays a role in development of the liver, kidney, and intestines. Decreased expression of this gene has been associated with monogenic autosomal dominant non-insulin-dependent diabetes mellitus type I (MODY I) and liver disease, e.g., cirrhosis. Dysregulated, e.g., increased, expression of this gene has been associated with hepatocellular carcinoma. The nucleotide and amino acid sequence of HNF4α is known and may be found in, for example, GenBank Accession Nos. NM_000457; NM_175914; NM_178849; NM_178850; NM_001030003; NM_001030004; NM_001258355; NM_001287182; NM_001287183; NM_001287184; XM_005260407; NP 000448.3; NP 787110.2; NP_849180.1; NP_849181.1; NP_001025174.1; NP_001025175.1; NP_001245284.1; NP_001274111.1; NP_001274112.1; NP_001274113.1; XP_005260464.1, the entire contents of each of which are incorporated herein by reference. The nucleotide sequence of the genomic region of Chromosome 20 which includes the endogenous promoters of HNF4α and the HNF4α coding sequence is also known and may be found in GenBank Accession No. NC_000020.10 (42984441 . . . 43061485).

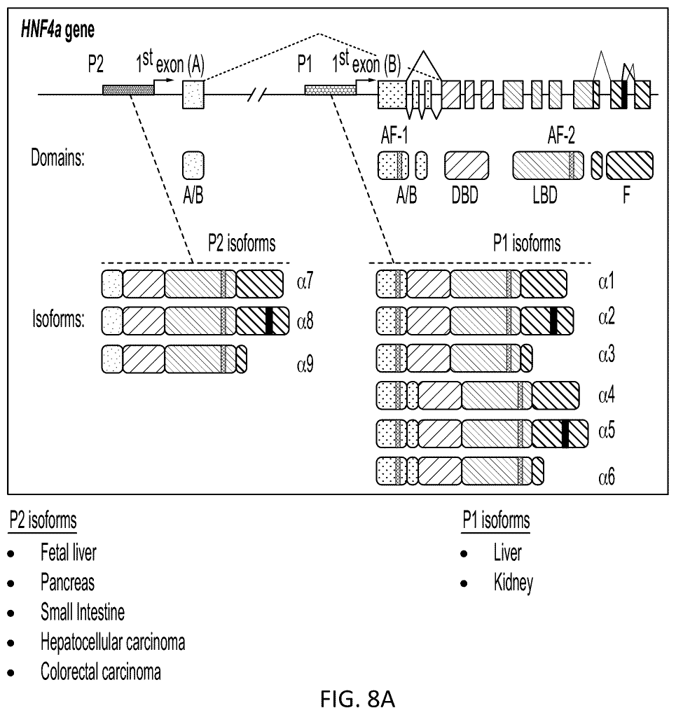

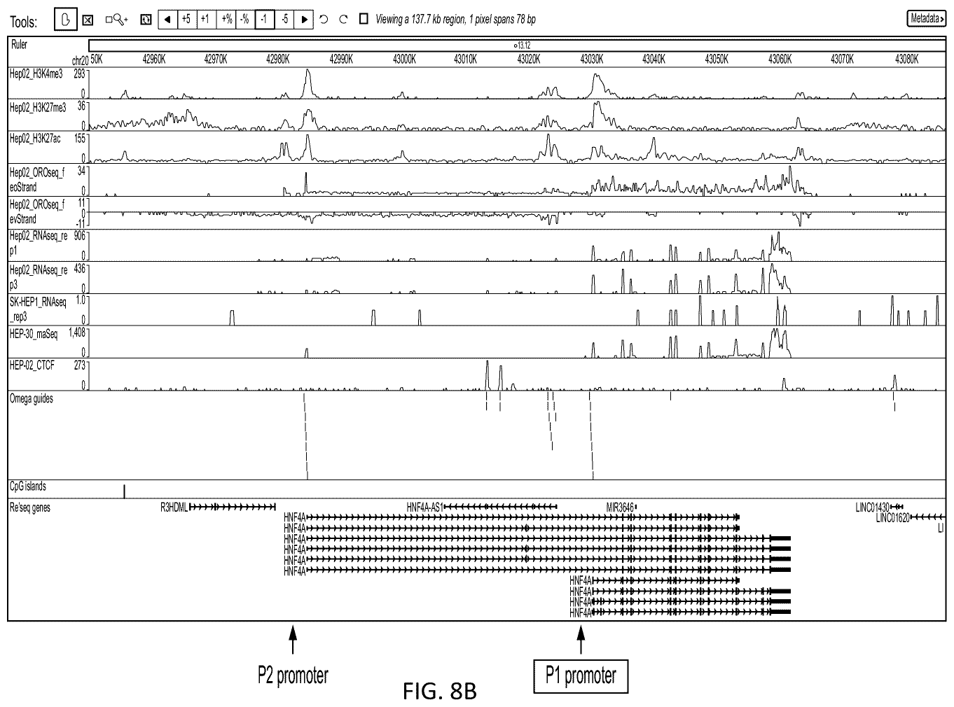

The HNF4α gene is located on chromosome 20, with transcription regulated by two promoters (P1 and P2) and alternative splicing variants, resulting in nine distinct isoforms (α1-α9) (FIGS. 8A-8B). The HNF4α locus is transcriptionally regulated through the use of two distinct promoters that are physically separated by more than 45 kb. Isoforms produced by the activity of the closer promoter are designated P1 whereas isoforms produced by the second and more distant promoter are designated P2. Isoforms most common in the liver are expressed from promoter 1 (P1), with isoforms from P2 most commonly found in fetal tissues, and in the adult kidney and small intestine.

HNF4α controls the expression of proteins necessary for the normal function of hepatocytes and other cell types in the liver. (Argemi J, et al. Defective HNF4αlpha-dependent gene expression as a driver of hepatocellular failure in alcoholic hepatitis. Nat Commun. 2019; 10(1):3126. doi:10.1038/s41467-019-11004-3; Nishikawa T, et al. Resetting the transcription factor network reverses terminal chronic hepatic failure. J Clin Invest. 2015; 125(4):1533-1544. doi:10.1172/JC173137). In addition, many of these proteins are secreted by liver cells and contribute to health systemically. For example, proteins such as albumin are required to transport nutrients, hormones, lipids, and small molecule drugs in the circulation.

The term “site-specific HNF4α disrupting agent,” as used herein, refers to any agent that specifically binds to a target HNF4α expression control region and, e.g., modulates expression of an HNF4α gene. The modulation of expression may be permanent or transient modulation. Site-specific HNF4α disruption agents of the invention may comprise a “site-specific HNF4α targeting moiety.”

As used herein, the term “site-specific HNF4α targeting moiety” refers to a moiety that specifically binds to an HNF4α expression control region, e.g., a transcriptional control region of an HNF4α gene, such as a promoter, an enhancer, or a repressor; or an HNF4α-associated anchor sequence, such as, for example within an HNF4α-associated anchor sequence-mediated conjunction.

Exemplary “site-specific HNF4α targeting moieties” include, but are not limited to, polyamides, nucleic acid molecules, such as RNA, DNA, or modified RNA or DNA, polypeptides, protein nucleic acid molecules, and fusion proteins.

As used herein, the terms “specific binding” or “specifically binds” refer to an ability to discriminate between possible binding partners in the environment in which binding is to occur. In some embodiments, a disrupting agent that interacts, e.g., preferentially interacts, with one particular target when other potential disrupting agents are present is said to “bind specifically” to the target (i.e., the expression control region) with which it interacts. In some embodiments, specific binding is assessed by detecting or determining the degree of association between the disrupting agent and its target; in some embodiments, specific binding is assessed by detecting or determining degree of dissociation of a disrupting agent-target complex. In some embodiments, specific binding is assessed by detecting or determining ability of the disrupting agent to compete with an alternative interaction between its target and another entity. In some embodiments, specific binding is assessed by performing such detections or determinations across a range of concentrations.

As used herein, the term “expression control region” or expression control domain’ refers to a region or domain present in a genomic DNA that modulates the expression of a target gene in a cell. A functionality associated with an expression control region may directly affect expression of a target gene, e.g., by recruiting or blocking recruitment of a transcription factor that would stimulate expression of the gene. A functionality associated with an expression control region may indirectly affect expression of a target gene, e.g., by introducing epigenetic modifications or recruiting other factors that introduce epigenetic modifications that induce a change in chromosomal topology that modulates expression of a target gene. Expression control regions may be upstream and/or downstream of the protein coding sequence of a gene and include, for example, transcriptional control elements, e.g., promoters, enhancers, or repressors; and anchor sequences, and anchor sequence-mediated conjunctions.

The term “transcriptional control element,” as used herein, refers to a nucleic acid sequence that controls transcription of a gene. Transcriptional control elements include, for example, anchor sequences, anchor sequence-mediated conjunctions, promoters, transcriptional enhancers, and transcriptional repressors.

A promoter is a region of DNA recognized by an RNA polymerase to initiate transcription of a particular gene and is generally located upstream of the 5′-end of the transcription start site of the gene.

A “transcriptional enhancer” increases gene transcription. A “transcriptional silencer” or “transcriptional repressor” decreases gene transcription. Enhancing and silencing sequences may be about 50-3500 base pairs in length and may influence gene transcription up to about 1 megabases away.

The term “gene,” as used herein, refers to a sequence of nucleotides that encode a molecule, such as a protein, that has a function. A gene contains sequences that are transcribed (e.g., a 3′UTR), sequences that are not transcribed (e.g., a promoter), sequences that are translated (e.g., an exon), and sequences that are not translated (e.g., intron).

As used herein, the term “target gene” means an HNF4α gene that is targeted for modulation, e.g., increase or decrease, of expression. In some embodiments, an HNF4α target gene is part of a targeted genomic complex (e.g. an HNF4α gene that has at least part of its genomic sequence as part of a target genomic complex, e.g. inside an anchor sequence-mediated conjunction), which genomic complex is targeted by one or more site-specific disrupting agents as described herein. In some embodiments, modulation comprises inhibition of expression of the target gene. In some embodiments, an HNF4α gene is modulated by contacting the HNF4α gene or a transcription control element operably linked to the HNF4α gene with one or more site-specific disrupting agents as described herein. In some embodiments, an HNF4α gene is aberrantly expressed (e.g., over-expressed) in a cell, e.g., a cell in a subject (e.g., a subject having an HNF4α-associated disease). In some embodiments, an HNF4α gene is aberrantly expressed (e.g., under-expressed) in a cell, e.g., a cell in a subject (e.g., a subject having an HNF4α-associated disease).

The term “anchor sequence” as used herein, refers to a nucleic acid sequence recognized by a nucleating agent that binds sufficiently to form an anchor sequence-mediated conjunction, e.g., a complex. In some embodiments, an anchor sequence comprises one or more CTCF binding motifs. In some embodiments, an anchor sequence is not located within a gene coding region. In some embodiments, an anchor sequence is located within an intergenic region. In some embodiments, an anchor sequence is not located within either of an enhancer or a promoter. In some embodiments, an anchor sequence is located at least 400 bp, at least 450 bp, at least 500 bp, at least 550 bp, at least 600 bp, at least 650 bp, at least 700 bp, at least 750 bp, at least 800 bp, at least 850 bp, at least 900 bp, at least 950 bp, or at least 1 kb away from any transcription start site. In some embodiments, an anchor sequence is located within a region that is not associated with genomic imprinting, monoallelic expression, and/or monoallelic epigenetic marks. In some embodiments, the anchor sequence has one or more functions selected from binding an endogenous nucleating polypeptide (e.g., CTCF), interacting with a second anchor sequence to form an anchor sequence mediated conjunction, or insulating against an enhancer that is outside the anchor sequence mediated conjunction. In some embodiments of the present invention, technologies are provided that may specifically target a particular anchor sequence or anchor sequences, without targeting other anchor sequences (e.g., sequences that may contain a nucleating agent (e.g., CTCF) binding motif in a different context); such targeted anchor sequences may be referred to as the “target anchor sequence”. In some embodiments, sequence and/or activity of a target anchor sequence is modulated while sequence and/or activity of one or more other anchor sequences that may be present in the same system (e.g., in the same cell and/or in some embodiments on the same nucleic acid molecule, e.g., the same chromosome) as the other targeted anchor sequence is not modulated. In some embodiments, the anchor sequence comprises or is a nucleating polypeptide binding motif. In some embodiments, the anchor sequence is adjacent to a nucleating polypeptide binding motif.

The term “anchor sequence-mediated conjunction” as used herein, refers to a DNA structure, in some cases, a complex, that occurs and/or is maintained via physical interaction or binding of at least two anchor sequences in the DNA by one or more polypeptides, such as nucleating polypeptides, or one or more proteins and/or a nucleic acid entity (such as RNA or DNA), that bind the anchor sequences to enable spatial proximity and functional linkage between the anchor sequences.

As used herein, the term “genomic complex” is a complex that brings together two genomic sequence elements that are spaced apart from one another on one or more chromosomes, via interactions between and among a plurality of protein and/or other components (potentially including, the genomic sequence elements). In some embodiments, the genomic sequence elements are anchor sequences to which one or more protein components of the complex bind. In some embodiments, a genomic complex may comprise an anchor sequence-mediated conjunction. In some embodiments, a genomic sequence element may be or comprise a CTCF binding motif, a promoter and/or an enhancer. In some embodiments, a genomic sequence element includes at least one or both of a promoter and/or regulatory region (e.g., an enhancer). In some embodiments, complex formation is nucleated at the genomic sequence element(s) and/or by binding of one or more of the protein component(s) to the genomic sequence element(s). As will be understood by those skilled in the art, in some embodiments, co-localization (e.g., conjunction) of the genomic sites via formation of the complex alters DNA topology at or near the genomic sequence element(s), including, in some embodiments, between them. In some embodiments, a genomic complex comprises an anchor sequence-mediated conjunction, which comprises one or more loops. In some embodiments, a genomic complex as described herein is nucleated by a nucleating polypeptide such as, for example, CTCF and/or Cohesin. In some embodiments, a genomic complex as described herein may include, for example, one or more of CTCF, Cohesin, non-coding RNA (e.g., eRNA), transcriptional machinery proteins (e.g., RNA polymerase, one or more transcription factors, for example selected from the group consisting of TFIIA, TFIIB, TFIID, TFIIE, TFIIF, TFIIH, etc.), transcriptional regulators (e.g., Mediator, P300, enhancer-binding proteins, repressor-binding proteins, histone modifiers, etc.), etc. In some embodiments, a genomic complex as described herein includes one or more polypeptide components and/or one or more nucleic acid components (e.g., one or more RNA components), which may, in some embodiments, be interacting with one another and/or with one or more genomic sequence elements (e.g., anchor sequences, promoter sequences, regulatory sequences (e.g., enhancer sequences)) so as to constrain a stretch of genomic DNA into a topological configuration (e.g., a loop) that the stretch of genomic DNA does not adopt when the complex is not formed.

An “effector molecule,” as used herein, refers to a molecule that is able to regulate a biological activity, such as enzymatic activity, gene expression, anchor sequence-mediated conjunction or cell signaling. Exemplary effectors are described in Section II, below, and in some embodiment include, for example, nucleases, physical blockers, epigenetic recruiters, e.g., a transcriptional enhancer or a transcriptional repressor, and epigenetic CpG modifiers, e.g., a DNA methylase, a DNA demethylase, a histone modifying agent, or a histone deacetylase, and combinations of any of the foregoing.

### II. SITE-SPECIFIC HNF4α DISRUPTING AGENTS OF THE INVENTION

The present invention provides site-specific HNF4α disrupting agents which, in one aspect of the invention include a site-specific HNF4α targeting moiety which targets an HNF4α expression control region. In another aspect, the site-specific disrupting agents of the invention include a site-specific HNF4α targeting moiety which targets an HNF4α expression control region and an effector molecule. As will be appreciated by one of ordinary skill in the art, such disrupting agents are site-specific and, thus, specifically bind to an HNF4α expression control region (e.g., one or more transcriptional control elements and/or one or more target anchor sequences), e.g., within a cell and not to non-targeted expression control regions (e.g., within the same cell).

The site-specific HNF4α disrupting agents of the invention comprise a site-specific HNF4α targeting moiety targeting an HNF4α expression control region. The expression control region targeted by the site-specific targeting moiety may be, for example, a transcriptional control element or an anchor sequence, such as an anchor sequence within an anchor-mediated conjunction.

Thus, site-specific HNF4α disrupting agents of the invention may modulate expression of a gene, i.e., HNF4α, e.g., by modulating expression of the gene from an endogenous promoter, an enhancer, or an repressor, may alter methylation of the control region, may alter at least one anchor sequence; may alter at least one conjunction nucleating molecule binding site, such as by altering binding affinity for the conjunction nucleating molecule; may alter an orientation of at least one common nucleotide sequence, such as a CTCF binding motif by, e.g., substitution, addition or deletion in at least one anchor sequence, such as a CTCF binding motif.

In certain embodiments, the site-specific disrupting agents and compositions described herein target an expression control region comprising one or more HNF4α-specific transcriptional control elements to modulate expression in a cell. HNF4α-specific transcriptional control elements that can be targeted include HNF4α-specific promoters, HNF4α-specific enhancers, and HNF4α-specific repressors. In one embodiment, an HNF4α-specific promoter substantially drives expression in cells of the liver, i.e., promoter 1. In one embodiment, an HNF4α-specific promoter substantially drives expression in cells of the pancreas, i.e., promoter 2. The nucleotide sequences of HNF4α promoter 1 and promoter 2 are known and may be found in, for example, GenBank Accession No. NC_000020.10 (42984441 . . . 43061485).

For example, a site-specific disrupting agent may include a site-specific targeting moiety, e.g., a nucleic acid molecule encoding a DNA-binding domain of a Transcription activator-like effector (TALE) polypeptide or a zinc finger (ZNF) polypeptide, or fragment thereof, that specifically targets and binds to an HNF4α expression control region, such as an HNF4α endogenous promoter region, e.g., promoter 1, and an effector molecule, such as an effector molecule that includes a transcriptional enhancer or transcriptional repressor that modulates, e.g., enhances or represses, expression of a target gene from an endogenous promoter to modulate gene expression. In one embodiment, the disrupting agent is “bicistronic nucleic acid molecule,” i.e., capable of making two fusion proteins from a single messenger RNA molecule, a first and a second site-specific targeting moiety, e.g., a nucleic acid molecule encoding a DNA-binding domain of a Transcription activator-like effector (TALE) polypeptide or a zinc finger (ZNF) polypeptide, or fragment thereof, that specifically targets and binds to an HNF4α expression control region, such as an HNF4α endogenous promoter region, e.g., promoter 1, and an effector molecule, such as an effector molecule that includes a transcriptional enhancer or transcriptional repressor that modulates, e.g., enhances or represses, expression of a target gene from an endogenous promoter to modulate gene expression.

In some embodiments of the invention, a site-specific disrupting agent may include a site-specific targeting moiety, e.g., a nucleic acid molecule such as a guide RNA targeting an HNF4α endogenous promoter region, e.g., promoter 1, and an effector molecule, such as an effector molecule that includes a transcriptional enhancer or transcriptional repressor that modulates, e.g., enhances or represses, expression of a target gene from an endogenous promoter to modulate gene expression.

In certain embodiments of the invention, the site-specific disrupting agents and compositions described herein target an expression control region comprising one or more HNF4α-associated anchor sequences, e.g., within an anchor sequence-mediated conjunction, comprising a first and a second HNF4α-associated anchor sequence to alter a two-dimensional chromatin structure (e.g., anchor sequence-mediated conjunctions in order to modulate expression in a cell, e.g., a cell within a subject, e.g., by modifying anchor sequence-mediated conjunctions in DNA, e.g., genomic DNA.

In one aspect, the invention includes a site-specific HNF4α disrupting agent comprising a site-specific HNF4α targeting moiety which targets an HNF4α expression control region comprising one or more HNF4α-associated anchor sequences within an anchor sequence-mediated conjunction. The disrupting agent binds, e.g., specifically binds, a specific anchor sequence-mediated conjunction to alter a topology of the anchor sequence-mediated conjunction, e.g., an anchor sequence-mediated conjunction having a physical interaction of two or more DNA loci bound by a conjunction nucleating molecule.

The formation of an anchor sequence-mediated conjunction may force transcriptional control elements to interact with an HNF4α gene or spatially constrain the activity of the transcriptional control elements. Altering anchor sequence-mediated conjunctions, therefore, allows for modulating HNF4α expression without altering the coding sequences of the HNF4α gene being modulated.

In some embodiments, the site-specific disrupting agents and compositions of the invention modulate expression of an HNF4α gene associated with an anchor sequence-mediated conjunction by physically interfering between one or more anchor sequences and a conjunction nucleating molecule. For example, a DNA binding small molecule (e.g., minor or major groove binders), peptide (e.g., zinc finger, TALE, novel or modified peptide), protein (e.g., CTCF, modified CTCF with impaired CTCF binding and/or cohesion binding affinity), or nucleic acids (e.g., ssDNA, modified DNA or RNA, peptide oligonucleotide conjugates, locked nucleic acids, bridged nucleic acids, polyamides, and/or triplex forming oligonucleotides) may physically prevent a conjunction nucleating molecule from interacting with one or more anchor sequences to modulate HNF4α gene expression.

In some embodiments, the site-specific disrupting agents and compositions of the invention modulate expression of an HNF4α gene associated with an anchor sequence-mediated conjunction by modification of an anchor sequence, e.g., epigenetic modifications, e.g., histone protein modifications, or genomic editing modifications. For example, one or more anchor sequences associated with an anchor sequence-mediated conjunction comprising an HNF4α gene may be targeted for methylation modification by a DNA methyltransferase, e.g., dCas9-methyltransferase fusion, e.g., antisense oligonucleotide-enzyme fusion, to modulate expression of the gene.

In some embodiments, the site-specific disrupting agents and compositions of the invention modulate expression of an HNF4α gene associated with an anchor sequence-mediated conjunction, e.g., activate or represses transcription, e.g., induces epigenetic changes to chromatin.

In some embodiments, an anchor sequence-mediated conjunction includes one or more anchor sequences, an HNF4α gene, and one or more transcriptional control elements, such as an enhancing or silencing element. In some embodiments, the transcriptional control element is within, partially within, or outside the anchor sequence-mediated conjunction.

In one embodiment, the anchor sequence-mediated conjunction comprises a loop, such as an intra-chromosomal loop. In certain embodiments, the anchor sequence-mediated conjunction has a plurality of loops. One or more loops may include a first anchor sequence, a nucleic acid sequence, a transcriptional control element, and a second anchor sequence. In another embodiment, at least one loop includes, in order, a first anchor sequence, a transcriptional control element, and a second anchor sequence; or a first anchor sequence, a nucleic acid sequence, and a second anchor sequence. In yet another embodiment, either one or both of the nucleic acid sequences and the transcriptional control element is located within or outside the loop. In still another embodiment, one or more of the loops comprises a transcriptional control element.

In some embodiments, the anchor sequence-mediated conjunction includes a TATA box, a CAAT box, a GC box, or a CAP site.

In some embodiments, the anchor sequence-mediated conjunction comprises a plurality of loops, and where the anchor sequence-mediated conjunction comprises at least one of an anchor sequence, a nucleic acid sequence, and a transcriptional control element in one or more of the loops.

In one aspect, the site-specific disrupting agents and compositions of the invention may introduce a targeted alteration to an anchor sequence-mediated conjunction to modulate expression of a nucleic acid sequence with a disrupting agent that binds the anchor sequence. In some embodiments, the anchor sequence-mediated conjunction is altered by targeting one or more nucleotides within the anchor sequence-mediated conjunction for substitution, addition or deletion.

In some embodiments, expression, e.g., transcription, is activated by inclusion of an activating loop or exclusion of a repressive loop. In one such embodiment, the anchor sequence-mediated conjunction comprises a transcriptional control sequence that increases transcription of a nucleic acid sequence, e.g., such an HNF4α encoding nucleic acid. In another such embodiment, the anchor sequence-mediated conjunction excludes a transcriptional control element that decreases expression, e.g., transcription, of a nucleic acid sequence, e.g., such an HNF4α encoding nucleic acid.

In some embodiments, expression, e.g., transcription, is repressed by inclusion of a repressive loop or exclusion of an activating loop. In one such embodiment, the anchor sequence-mediated conjunction includes a transcriptional control element that decreases expression, e.g., transcription, of a nucleic acid sequence, e.g., such an HNF4α encoding nucleic acid sequence. In another such embodiment, the anchor sequence-mediated conjunction excludes a transcriptional control sequence that increases transcription of a nucleic acid sequence, e.g., such an HNF4α encoding nucleic acid.

Each anchor sequence-mediated conjunction comprises one or more anchor sequences, e.g., a plurality. Anchor sequences can be manipulated or altered to disrupt naturally occurring loops or form new loops (e.g., to form exogenous loops or to form non-naturally occurring loops with exogenous or altered anchor sequences). Such alterations modulate HNF4α gene expression by changing the 2-dimensional structure of DNA containing all or a portion of an HNF4α gene, e.g., by thereby modulating the ability of the HNF4α gene to interact with transcriptional control elements (e.g., enhancing and silencing/repressive sequences). In some embodiments, the chromatin structure is modified by substituting, adding or deleting one or more nucleotides within an anchor sequence of the anchor sequence-mediated conjunction.

The anchor sequences may be non-contiguous with one another. In embodiments with noncontiguous anchor sequences, the first anchor sequence may be separated from the second anchor sequence by about 500 bp to about 500 Mb, about 750 bp to about 200 Mb, about 1 kb to about 100 Mb, about 25 kb to about 50 Mb, about 50 kb to about 1 Mb, about 100 kb to about 750 kb, about 150 kb to about 500 kb, or about 175 kb to about 500 kb. In some embodiments, the first anchor sequence is separated from the second anchor sequence by about 500 bp, 600 bp, 700 bp, 800 bp, 900 bp, 1 kb, 5 kb, 10 kb, 15 kb, 20 kb, 25 kb, 30 kb, 35 kb, 40 kb, 45 kb, 50 kb, 55 kb, 60 kb, 65 kb, 70 kb, 75 kb, 80 kb, 85 kb, 90 kb, 95 kb, 100 kb, 125 kb, 150 kb, 175 kb, 200 kb, 225 kb, 250 kb, 275 kb, 300 kb, 350 kb, 400 kb, 500 kb, 600 kb, 700 kb, 800 kb, 900 kb, 1 Mb, 2 Mb, 3 Mb, 4 Mb, 5 Mb, 6 Mb, 7 Mb, 8 Mb, 9 Mb, 10 Mb, 15 Mb, 20 Mb, 25 Mb, 50 Mb, 75 Mb, 100 Mb, 200 Mb, 300 Mb, 400 Mb, 500 Mb, or any size therebetween.

In one embodiment, the anchor sequence comprises a common nucleotide sequence, e.g., a CTCF-binding motif:

(SEQ ID NO: 64)
N(T/C/G)N(G/A/T)CC(A/T/G)(C/G)(C/T/A)AG(G/A)(G/T)
GG(C/A/T)(G/A)(C/G)(C/T/A)(G/A/C),

where N is any nucleotide.

A CTCF-binding motif may also be in the opposite orientation, e.g.,

(SEQ ID NO: 65)
(G/A/C)(C/T/A)(C/G)(G/A)(C/A/T)GG(G/T)(G/A)GA
(C/T/A)(C/G)(A/T/G)CC(G/A/T)N(T/C/G)N.

In one embodiment, the anchor sequence comprises SEQ ID NO: 64 or SEQ ID NO:65 or a nucleotide sequence at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to either SEQ ID NO: 64 or SEQ ID NO:65.

In some embodiments, the anchor sequence-mediated conjunction comprises at least a first anchor sequence and a second anchor sequence. The first anchor sequence and second anchor sequence may each comprise a common nucleotide sequence, e.g., each comprises a CTCF binding motif. In some embodiments, the first anchor sequence and second anchor sequence comprise different sequences, e.g., the first anchor sequence comprises a CTCF binding motif and the second anchor sequence comprises an anchor sequence other than a CTCF binding motif. In some embodiments, each anchor sequence comprises a common nucleotide sequence and one or more flanking nucleotides on one or both sides of the common nucleotide sequence.

Two CTCF-binding motifs (e.g., contiguous or non-contiguous CTCF binding motifs) that can form a conjunction may be present in the genome in any orientation, e.g., in the same orientation (tandem) either 5′->3′ (left tandem, e.g., the two CTCF-binding motifs that comprise SEQ ID NO: 64) or 3′->5′ (right tandem, e.g., the two CTCF-binding motifs comprise SEQ ID NO: 65), or convergent orientation, where one CTCF-binding motif comprises SEQ ID NO: 64 and the other comprises SEQ ID NO: 65. CTCFBSDB 2.0: Database For CTCF binding motifs And Genome Organization can be used to identify CTCF binding motifs associated with a target gene, e.g., HNF4α.

In some embodiments, the anchor sequence-mediated conjunction is altered by changing an orientation of at least one common nucleotide sequence, e.g., a conjunction nucleating molecule binding site.

In some embodiments, the anchor sequence comprises a conjunction nucleating molecule binding site, e.g., CTCF binding motif, and site-specific disrupting agent of the invention introduces an alteration in at least one conjunction nucleating molecule binding site, e.g. altering binding affinity for the conjunction nucleating molecule.

In some embodiments, the anchor sequence-mediated conjunction is altered by introducing an exogenous anchor sequence. Addition of a non-naturally occurring or exogenous anchor sequence to form or disrupt a naturally occurring anchor sequence-mediated conjunction, e.g., by inducing a non-naturally occurring loop to form that alters transcription of the nucleic acid sequence.

In some embodiments, the anchor sequence-mediated conjunction comprises an HNF4α gene, and one or more, e.g., 2, 3, 4, 5, or other genes other than the HNF4α gene.

In some embodiments, the anchor sequence-mediated conjunction is associated with one or more, e.g., 2, 3, 4, 5, or more, transcriptional control elements. In some embodiments, the HNF4α gene is noncontiguous with one or more of the transcriptional control elements. In some embodiments where the HNF4α gene is non-contiguous with the transcriptional control element, the gene may be separated from one or more transcriptional control elements by about 100 bp to about 500 Mb, about 500 bp to about 200 Mb, about 1 kb to about 100 Mb, about 25 kb to about 50 Mb, about 50 kb to about 1 Mb, about 100 kb to about 750 kb, about 150 kb to about 500 kb, or about 175 kb to about 500 kb. In some embodiments, the gene is separated from the transcriptional control element by about 100 bp, 300 bp, 500 bp, 600 bp, 700 bp, 800 bp, 900 bp, 1 kb, 5 kb, 10 kb, 15 kb, 20 kb, 25 kb, 30 kb, 35 kb, 40 kb, 45 kb, 50 kb, 55 kb, 60 kb, 65 kb, 70 kb, 75 kb, 80 kb, 85 kb, 90 kb, 95 kb, 100 kb, 125 kb, 150 kb, 175 kb, 200 kb, 225 kb, 250 kb, 275 kb, 300 kb, 350 kb, 400 kb, 500 kb, 600 kb, 700 kb, 800 kb, 900 kb, 1 Mb, 2 Mb, 3 Mb, 4 Mb, 5 Mb, 6 Mb, 7 Mb, 8 Mb, 9 Mb, 10 Mb, 15 Mb, 20 Mb, 25 Mb, 50 Mb, 75 Mb, 100 Mb, 200 Mb, 300 Mb, 400 Mb, 500 Mb, or any size therebetween.

In some embodiments, the type of anchor sequence-mediated conjunction may help to determine how to modulate gene expression, e.g., choice of site-specific targeting moiety, by altering the anchor sequence-mediated conjunction. For example, some types of anchor sequence-mediated conjunctions comprise one or more transcription control elements within the anchor sequence-mediated conjunction. Disruption of such an anchor sequence-mediated conjunction by disrupting the formation of the anchor sequence-mediated conjunction, e.g., altering one or more anchor sequences, is likely to decrease transcription of an HNF4α gene within the anchor sequence-mediated conjunction.

In some embodiments, expression of the HNF4α gene is regulated, modulated, or influenced by one or more transcriptional control elements associated with the anchor sequence-mediated conjunction. In some embodiments, the anchor sequence-mediated conjunction comprises an HNF4α gene and one or more transcriptional control elements. For example, the HNF4α gene and one or more transcriptional control sequences are located within, at least partially, an anchor sequence-mediated conjunction, e.g., a Type 1 anchor sequence-mediated conjunction. The anchor sequence-mediated conjunction may also be referred to as a “Type 1, EP subtype.” In some embodiments, the HNF4α gene has a defined state of expression, e.g., in its native state, e.g., in a diseased state. For example, the HNF4α gene may have a high level of expression. By disrupting the anchor sequence-mediated conjunction, expression of the HNF4α gene may be decreased, e.g., decreased transcription due to conformational changes of the DNA previously open to transcription within the anchor sequence-mediated conjunction, e.g., decreased transcription due to conformational changes of the DNA creating additional distance between the HNF4α gene and the enhancing sequences. In one embodiment, both the HNF4α gene associated and one or more transcriptional control sequences, e.g., enhancing sequences, reside inside the anchor sequence-mediated conjunction. Disruption of the anchor sequence-mediated conjunction decreases expression of the HNF4α gene. In one embodiment, the HNF4α gene associated with the anchor sequence-mediated conjunction is accessible to one or more transcriptional control elements that reside inside, at least partially, the anchor sequence-mediated conjunction.

In some embodiments, expression of the HNF4α gene is regulated, modulated, or influenced by one or more transcriptional control elements associated with, but inaccessible due to the anchor sequence-mediated conjunction. For example, the anchor sequence-mediated conjunction associated with an HNF4α gene disrupts the ability of one or more transcriptional control elements to regulate, modulate, or influence expression of the HNF4α gene. The transcriptional control sequences may be separated from the HNF4α gene, e.g., reside on the opposite side, at least partially, e.g., inside or outside, of the anchor sequence-mediated conjunction as the HNF4α gene, e.g., the HNF4α gene is inaccessible to the transcriptional control elements due to proximity of the anchor sequence-mediated conjunction. In some embodiments, one or more enhancing sequences are separated from the HNF4α gene by the anchor sequence-mediated conjunction, e.g., a Type 2 anchor sequence-mediated conjunction.

In some embodiments, the HNF4α gene is inaccessible to one or more transcriptional control elements due to the anchor sequence-mediated conjunction, and disruption of the anchor sequence-mediated conjunction allows the transcriptional control element to regulate, modulate, or influence expression of the HNF4α gene. In one embodiment, the HNF4α gene is inside and outside the anchor sequence-mediated conjunction and inaccessible to the one or more transcriptional control elements. Disruption of the anchor sequence-mediated conjunction increases access of the transcriptional control elements to regulate, modulate, or influence expression of the HNF4α gene, e.g., the transcriptional control elements increase expression of the HNF4α gene. In one embodiment, the HNF4α gene is inside the anchor sequence-mediated conjunction and inaccessible to the one or more transcriptional control elements residing outside, at least partially, the anchor sequence-mediated conjunction. Disruption of the anchor sequence-mediated conjunction increases expression of the HNF4α gene. In one embodiment, the HNF4α gene is outside, at least partially, the anchor sequence-mediated conjunction and inaccessible to the one or more transcriptional control elements residing inside the anchor sequence-mediated conjunction. Disruption of the anchor sequence-mediated conjunction increases expression of the HNF4α gene.

### A. HNF4α Site-Specific Targeting Moieties

The site-specific HNF4α targeting moieties of the invention target an HNF4α expression control region and may comprise a polymer or polymeric molecule, such as a polyamide (i.e., a molecule of repeating units linked by amide binds, e.g., a polypeptide), a polymer of nucleotides (such as a guide RNA, a nucleic acid molecule encoding a TALE polypeptide or a zinc finger polypeptides), a peptide nucleic acid (PNA), or a polymer of amino acids, such as a peptide or polypeptide, e.g., a fusion protein, etc. Suitable site-specific HNF4α targeting moieties, compositions, and methods of use of such agents and compositions are described below and in PCT Publication WO 2018/049073, the entire contents of which are expressly incorporated herein by reference.

In one embodiment, a site specific disrupting agent of the invention comprises a site-specific HNF4α targeting moiety comprising a nucleic acid molecule encoding a polypeptide, such as a DNA-binding domain, of a Transcription activator-like effector (TALE) polypeptide or a zinc finger (ZNF) polypeptide, or fragment thereof, that is engineered to specifically target an HNF4α expression control region to modulate expression of an HNF4α gene.

In another embodiment, a site-specific disrupting agent of the invention comprises a site-specific HNF4α targeting moiety comprising a nucleic acid molecule, such as a guide RNA (or gRNA) or a guide RNA and an effector, or fragment thereof, or nucleic acid molecule encoding an effector, or fragment thereof.

In another embodiment, a site-specific disrupting agent of the invention comprises a site-specific HNF4α targeting moiety comprising a polynucleotide, such as a PNA, e.g., a nucleic acid gRNA linked to an effector polypeptide, or fragment thereof.

In another embodiment, a site-specific disrupting agent of the invention comprises a site-specific HNF4α targeting moiety comprising a fusion molecule, such as a nucleic acid molecule encoding a DNA-binding domain, of a Transcription activator-like effector (TALE) polypeptide or a zinc finger (ZNF) polypeptide, or fragment thereof, and an effector.

In one embodiment, such site-specific disrupting agents comprise a second fusion protein, wherein the second fusion protein comprises a second site-specific HNF4α targeting moiety which targets a second HNF4α expression control region and a second effector molecule, wherein the second HNF4α expression control region is different than the HNF4α expression control region.

In another embodiment, a site-specific disrupting agent of the invention comprises a site-specific HNF4α targeting moiety comprising a fusion molecule, such as a nucleic acid molecule encoding a protein comprising a Cas polypeptide and, e.g., an epigenetic recruiter or an epigenetic CpG modifier.

In yet, another embodiment, a site-specific disrupting agent of the invention comprises a site-specific HNF4α targeting moiety comprising a fusion molecule, such as fusion protein comprising a Cas polypeptide and, e.g., an epigenetic recruiter or an epigenetic CpG modifier.

As used herein, in its broadest sense, the term “nucleic acid” refers to any compound and/or substance that is or can be incorporated into an oligonucleotide chain. In some embodiments, a nucleic acid is a compound and/or substance that is or can be incorporated into a polynucleotide chain via a phosphodiester linkage. As will be clear from context, in some embodiments, “nucleic acid” refers to an individual nucleic acid residue (e.g., a nucleotide and/or nucleoside); in some embodiments, “nucleic acid” refers to a polynucleotide chain comprising individual nucleic acid residues. In some embodiments, a “nucleic acid” is or comprises RNA; in some embodiments, a “nucleic acid” is or comprises DNA. In some embodiments, a “nucleic acid” is a “mixmer” comprising locked nucleic acid molecules and deoxynucleic acid molecules. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present invention. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is partly or wholly single stranded; in some embodiments, a nucleic acid is partly or wholly double stranded. In some embodiments a nucleic acid has a nucleotide sequence comprising at least one element that encodes, or is the complement of a sequence that encodes, a polypeptide. In some embodiments, a nucleic acid has enzymatic activity.

As used herein, the terms “peptide,” “polypeptide,” and “protein” refer to a compound comprised of amino acid residues covalently linked by peptide bonds, or by means other than peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds or by means other than peptide bonds. As used herein, the term refers to both short chains, which also commonly are referred to in the art as peptides, oligopeptides and oligomers, for example, and to longer chains, which generally are referred to in the art as proteins, of which there are many types.

In certain embodiments, a polypeptide is or may comprise a chimeric or “fusion protein.” As used herein, a “chimeric protein” or “fusion protein” comprises all or part (preferably a biologically active part) of a first protein operably linked to a heterologous second polypeptide (i.e., a polypeptide other than the first protein). Within the fusion protein, the term “operably linked” is intended to indicate that the first protein or segment thereof and the heterologous polypeptide are fused in-frame to each other. The heterologous polypeptide can be fused to the amino-terminus or the carboxyl-terminus of the first protein or segment.

A “polyamide” is a polymeric molecule with repeating units linked by amide binds. Proteins are examples of naturally occurring polyamides. In some embodiments, a polyamide comprises a peptide nucleic acid (PNA).

A “peptide nucleic acid” (“PNA”) is a molecule in which one or more amino acid units in the PNA have an amide containing backbone, e.g., aminoethyl-glycine, similar to a peptide backbone, with a nucleic acid side chain in place of the amino acid side chain. Peptide nucleic acids (PNA) are known to hybridize complementary DNA and RNA with higher affinity than their oligonucleotide counterparts. This character of PNA not only makes them a stable hybrid with the nucleic acid side chains, but at the same time, the neutral backbone and hydrophobic side chains result in a hydrophobic unit within the polypeptide. The nucleic acid side chain includes, but is not limited to, a purine or a pyrimidine side chain such as adenine, cytosine, guanine, thymine and uracil. In one embodiment, the nucleic acid side chain includes a nucleoside analog as described herein.

In one embodiment, a site-specific HNF4α targeting moiety of the invention comprises a polyamide. Suitable polyamides for use in the agents and compositions of the invention are known in the art.

In one embodiment, a site-specific HNF4α targeting moiety of the invention comprises a polynucleotide. In some embodiments, the nucleotide sequence of the polynucleotide encodes an HNF4α gene or an HNF4α expression product. In some embodiments, the nucleotide sequence of the polynucleotide does not include an HNF4α coding sequence or an HNF4α expression product. For example, in some embodiments, a site-specific HNF4α targeting moiety of the invention comprises a polynucleotide that hybridizes to a target expression control region, e.g., a promoter or an anchor sequence. In some embodiments, the nucleotide sequence of the polynucleotide is a complement of a target anchor sequence, or has a sequence that is at least 80%, at least 85%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a complement of the target sequence.

The polynucleotides of the invention may include deoxynucleotides, ribonucleotides, modified deoxynucleotides, modified ribonucleotides (e.g., chemical modifications, such as modifications that alter the backbone linkages, sugar molecules, and/or nucleic acid bases), and artificial nucleic acids. In some embodiments, the polynucleotide includes, but is not limited to, genomic DNA, cDNA, peptide nucleic acids (PNA) or peptide oligonucleotide conjugates, locked nucleic acids (LNA), bridged nucleic acids (BNA), polyamides, triplex forming oligonucleotides, modified DNA, antisense DNA oligonucleotides, tRNA, mPvNA, rPvNA, modified RNA, miRNA, gRNA, and siRNA or other RNA or DNA molecules.

In some embodiments, the polynucleotides of the invention have a length from about 2 to about 5000 nts, about 10 to about 100 nts, about 50 to about 150 nts, about 100 to about 200 nts, about 150 to about 250 nts, about 200 to about 300 nts, about 250 to about 350 nts, about 300 to about 500 nts, about 10 to about 1000 nts, about 50 to about 1000 nts, about 100 to about 1000 nts, about 1000 to about 2000 nts, about 2000 to about 3000 nts, about 3000 to about 4000 nts, about 4000 to about 5000 nts, or any range therebetween.

The polynucleotides of the invention may include nucleosides, e.g., purines or pyrimidines, e.g., adenine, cytosine, guanine, thymine and uracil. In some embodiments, the polynucleotides includes one or more nucleoside analogs. The nucleoside analog includes, but is not limited to, a nucleoside analog, such as 5-fluorouracil; 5-bromouracil, 5-chlorouracil, 5-iodouracil, hypoxanthine, xanthine, 4-acetylcytosine, 4-methylbenzimidazole, 5-(carboxyhydroxylmethyl) uracil, 5-carboxymethylaminomethyl-2-thiouridine, 5-carboxymethylaminomethyluracil, dihydrouracil, dihydrouridine, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine, 3-nitropyrrole, inosine, thiouridine, queuosine, wyosine, diaminopurine, isoguanine, isocytosine, diaminopyrimidine, 2,4-difluorotoluene, isoquinoline, pyrrolo[2,3-]pyridine, and any others that can base pair with a purine or a pyrimidine side chain.

In some embodiments, the site-specific HNF4α targeting moieties of the invention comprise a polynucleotide encoding a polypeptide that comprises a DNA-binding domain (DBD), or fragment thereof, of a zinc finger or TALE, that is engineered to specifically target an HNF4α expression control region to modulate expression of an HNF4α gene.

The design and preparation of such zinc finger polypeptides which specifically bind to a DNA target region of interest, such as an HNF4α expression control region, is well known in the art. For example, zinc finger (ZNF) proteins contain a DNA binding domain that specifically binds a triplet of nucleotides. Thus, to design and prepare the site-specific HNF4α targeting moieties of the invention, a modular assembly process which includes combining separate zinc finger DNA binding domains that can each recognize a specific 3-basepair DNA sequence to generate 3-finger, 4-, 5-, 6-, 7-, or 8-zinc finger polypeptide that recognizes specific target sites ranging from 9 base pairs to 24 base pairs in length may be used. Another suitable method may include 2-finger modules to generate ZNF polynucleotides with up to six individual zinc fingers. See, e.g., Shukla V K, et al., Nature. 459 (7245) 2009: 437-41; Dreier B, et al., JBC. 280 (42) 2005: 35588-97; Dreier B, et al, JBC 276 (31) 2001: 29466-78; Bae K H, et al., Nature Biotechnology. 21 (3) 2003: 275-80.

In some embodiments, a site-specific HNF4α targeting moiety of the invention comprises a polynucleotide encoding a polypeptide that comprises a DNA-binding domain (DBD), or fragment thereof, of a zinc finger, that is engineered to specifically target an HNF4α expression control region to modulate expression of an HNF4α gene. Exemplary amino acid sequences encoding a zinc finger that binds to a nucleotide triplet suitable for use in the present invention are provide in Table 1A below. (See, e.g., Gersbach et al., Synthetic Zinc Finger Proteins: The Advent of Targeted Gene Regulation and Genome Modification Technologies).

TABLE 1A

| Amino Acid | | |
|---|---|---|
| Sequence of | | |
| Zing Finger | | |
| DNA Binding | | |
| Domain | Nucleotide | SEQ ID |
| (Finger) | Triplet | NO. |
| RKDALRG | TTG | 1 |
| TTGALTE | CTT | 2 |
| QRHHLVE | CTC | 3 |
| QNSTLTE | CTA | 4 |
| RNDALTE | CTG | 5 |
| HKNALQN | ATT | 6 |
| RRSACRR | ATC | 7 |
| QKSSLIA | ATA | 8 |
| RRDELNV | ATG | 9 |
| TSGSLVR | GTT | 10 |
| DPGALVR | GTC | 11 |
| QSSSLVR | GTA | 12 |
| RSDELVR | GTG | 13 |
| RLRDIQF | TCT | 14 |
| RSDERKR | TCC | 15 |
| RSDHLTT | TCA | 16 |
| RLRALDR | TCG | 17 |
| TKNSLTE | CCT | 18 |
| SKKHLAE | CCC | 19 |
| TSHSLTE | CCA | 20 |
| RNDTLTE | CCG | 21 |
| THLDLIR | ACT | 22 |
| DKKDLTR | ACC | 23 |
| SPADLTR | ACA | 24 |
| RTDTLRD | ACG | 25 |
| TSGELVR | GCT | 26 |
| DCRDLAR | GCC | 27 |
| QSGDLRR | GCA | 28 |
| RSDDLVR | GCG | 29 |
| ARGNLRT | TAT | 30 |
| SRGNLKS | TAC | 31 |
| QASNLIS | TAA | 32 |
| REDNLHT | TAG | 33 |
| TSGNLTE | CAT | 34 |
| SKKALTE | CAC | 35 |
| QSGNLTE | CAA | 36 |
| RADNLTE | CAG | 37 |
| TTGNLTV | AAT | 38 |
| DSGNLRV | AAC | 39 |
| QRANLRA | AAA | 40 |
| RKDNLKN | AAG | 41 |
| TSGNLVR | GAT | 42 |
| DPGNLVR | GAC | 43 |
| QSSNLVR | GAA | 44 |
| RSDNLVR | GAG | 45 |
| APKALGW | TGC | 46 |
| QAGHLAS | TGA | 47 |
| RSDHLTT | TGG | 48 |
| SRRTCRA | CGT | 49 |
| HTGHLLE | CGC | 50 |
| QSGHLTE | CGA | 51 |
| RSDKLTE | CGG | 52 |
| HRTTLTN | AGT | 53 |
| ERSHLRE | AGC | 54 |
| QLAHLRA | AGA | 55 |
| RSDHLTN | AGG | 56 |
| TSGHLVR | GGT | 57 |
| DPGHLVR | GGC | 58 |
| QRAHLER | GGA | 59 |
| RSDKLVR | GGG | 60 |

A zinc finger DNA binding domain comprises an N-terminal region and a C-terminal region with the “fingers” that bind to the target DNA sequence in between. The N-terminal region generally is 7 amino acids in length. The C-terminal region is generally 6 amino acids in length. Thus, the N-terminal region generally comprises the amino acid sequence of X₁X₂X₃X₄X₅X₆X₇. “X” can be any amino acid. In some embodiments, the N-terminal region comprises the exemplary amino acid sequence of LEPGEKP (SEQ ID NO: 309). “X” can be any amino acid. The C-terminal region generally comprises the amino acid sequence of X₂₅X₂₆X₂₇X₂₈X₂₉X₃₀. In certain embodiments, the C-terminal region comprises the exemplary amino acid sequence of TGKKTS (SEQ ID NO: 310)

Each finger in the DNA binding domain is flanked by a N-terminal backbone located to the N-terminus of the finger and a C-terminal backbone located to the C-terminus of the finger. The N-terminal backbone of the finger generally is 11 amino acids long with two conservative cysteines (C) locate at 3^{rd} and 6^{th} positions. Thus, the N-terminal backbone of the finger generally comprises the amino acid sequence of X₈X₉CX₁₀X₁₁CX₁₂X₁₃X₁₄X₁₅X₁₆. “X” can be any amino acid. The C-terminal backbone of the finger generally is 5 amino acids long with two conservative histines (H) located at 1^{st} and 5^{th} positions. Thus, the C-terminal backbone of the finger generally comprises the amino acid sequence of HX₁₇X₁₈X₁₉H. “X” can be any amino acid. In some embodiments, the N-terminal backbone comprises the exemplary amino acid sequence of YKCPECGKSFS (SEQ ID No. 61) and the C-terminal backbone comprises the exemplary amino acid sequence of HQRTH (SEQ ID No. 62). Two “fingers” are linked through a linker. A linker generally is 5 amino acids in length and comprises the amino acid sequence of X₂₀X₂₁X₂₂X₂₃X₂₄. “X” can be any amino acid. In certain embodiments, the linker comprises the exemplary amino acid sequence of TGEKP (SEQ ID No. 63). Thus, the zinc finger of a site specific HNF4α site-specific disrupting agent has a structure as follows: (N-terminal backbone-finger-C-terminal backbone-linker)ₙ and the zinc finger DNA binding domain of a site specific HNF4α site-specific disrupting agent has a structure as follows: [N-terminal region (N-terminal backbone-finger-C-terminal backbone-linker)ₙ-C-terminal region]. “N” represents the number of triplets of nucleotides to which the zinc finger DNA binding domain and, thus, to which the HNF4α site-specific disrupting agent binds.

The “finger” amino acid sequences of four nucleotide triplets are unknown, however, if such a triplet is identified in a target area of interest, two “linker span sequences”—linker span 1 and linker span 2—are useful to circumvent the issue. Linker span 1 is used to skip one base pair if a “finger” amino acid sequence of a triplet is not available. Linker span 2 is used to skip 2 base pairs if a “finger” amino acid sequence of a triplet is not available. Linker span 1 is generally 12 amino acids long. Linker span 2 is generally 16 amino acids long. Thus, linker span 1 generally comprises the amino acid sequence of X₃₁X₃₂X₃₃X₃₄X₃₅X₃₆X₃₇X₃₈X₃₉X₄₀X₄₁X₄₂. Linker span 2 generally comprises the amino acid sequence of X₄₃X₄₄X₄₅X₄₆X₄₇X₄₈X₄₉X₅₀X₅₁X₅₂X₅₃X₅₄X₅₅X₅₆X₅₇X₅₈. In some embodiments, linker span 1 comprises the amino acid sequence of THPRAPIPKPFQ (SEQ ID NO: 311). In certain embodiments, linker span 2 comprises the amino acid sequence of TPNPHRRTDPSHKPFQ (SEQ ID NO: 312). When linker span 1 and/or linker span 2 is used, the finger-linker span 1/span 2-finger comprises the structure as follows: N-terminal back bone-finger-C-terminal backbone-linker span 1/span 2-N-terminal backbone-finger-C-terminal backbone-linker.

Table 1B provides the amino acid sequence structure of exemplary zinc finger DNA binding domains of the disrupting agents comprising a zinc finger DNA binding domain described in the working examples below (see Table 6A). Table 10 also provides the nucleotide sequence of suitable target sequences in the expression control region, the amino acid sequences of exemplary zinc finger DNA binding domains suitable for use in the disrupting agents comprising a zinc finger DNA binding domain of the present invention as well as the amino acid sequence structure of the exemplary zinc finger DNA binding domains suitable for use in the disrupting agents comprising a zinc finger DNA binding domain of the present invention. The “X,” as used in Table 1B, represents any amino acid.

In some embodiments, a zinc finger DNA binding domain suitable for use in the disrupting agents of the invention comprises an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% amino acid identity to the entire amino acid sequence of any one of the zinc finger DNA binding domains provided in any one of Tables 6A and 10.

TABLE 1B

| Name of | | |
|---|---|---|
| Exemplary Zinc | | SEQ |
| Finger DNA | | ID |
| Binding Domain | Amino Acid Sequence Structure | NO: |
| ZF1 | X₁X₂X₃X₄X₅X₆X₇X₈X₉CX₁₀X₁₁CX₁₂X₁₃X₁₄X₁₅X₁₆RSDNLVRHX₁₇X₁₈ | 313 |
| | X₁₉HX₂₀X₂₁X₂₂X₂₃X₂₄X₈X₉CX₁₀X₁₁CX₁₂X₁₃X₁₄X₁₅X₁₆RSDHLTNHX₁₇ | |
| | X₁₈X₁₉HX₂₀X₂₁X₂₂X₂₃X₂₄X₈X₉CX₁₀X₁₁CX₁₂X₁₃X₁₄X₁₅X₁₆DPGHLVR | |
| | HX₁₇X₁₈X₁₉HX₂₀X₂₁X₂₂X₂₃X₂₄X₈X₉CX₁₀X₁₁CX₁₂X₁₃X₁₄X₁₅X₁₆QRAH | |
| | LERHX₁₇X₁₈X₁₉HX₂₀X₂₁X₂₂X₂₃X₂₄X₈X₉CX₁₀X₁₁CX₁₂X₁₃X₁₄X₁₅X₁₆SR | |
| | RTCRAHX₁₇X₁₈X₁₉HX₂₀X₂₁X₂₂X₂₃X₂₄X₈X₉CX₁₀X₁₁CX₁₂X₁₃X₁₄X₁₅X₁₆ | |
| | RSDKLTEHX₁₇X₁₈X₁₉HX₂₀X₂₁X₂₂X₂₃X₂₄X₈X₉CX₁₀X₁₁CX₁₂X₁₃X₁₄X₁₅ | |
| | X₁₆RNDTLTEHX₁₇X₁₈X₁₉HX₂₀X₂₁X₂₂X₂₃X₂₄X₂₅X₂₆X₂₇X₂₈X₂₉X₃₀ | |
| ZF2 | X₁X₂X₃X₄X₅X₆X₇X₈X₉CX₁₀X₁₁CX₁₂X₁₃X₁₄X₁₅X₁₆QSSNLVRHX₁₇X₁₈ | 314 |
| | X₁₉HX₂₀X₂₁X₂₂X₂₃X₂₄X₈X₉CX₁₀X₁₁CX₁₂X₁₃X₁₄X₁₅X₁₆SKKHLAEHX₁₇ | |
| | X₁₈X₁₉HX₂₀X₂₁X₂₂X₂₃X₂₄X₈X₉CX₁₀X₁₁CX₁₂X₁₃X₁₄X₁₅X₁₆SKKALTEH | |
| | X₁₇X₁₈X₁₉HX₂₀X₂₁X₂₂X₂₃X₂₄X₈X₉CX₁₀X₁₁CX₁₂X₁₃X₁₄X₁₅X₁₆DCRDL | |
| | ARHX₁₇X₁₈X₁₉HX₂₀X₂₁X₂₂X₂₃X₂₄X₈X₉CX₁₀X₁₁CX₁₂X₁₃X₁₄X₁₅X₁₆DP | |
| | GHLVRHX₁₇X₁₈X₁₉HX₂₀X₂₁X₂₂X₂₃X₂₄X₈X₉CX₁₀X₁₁CX₁₂X₁₃X₁₄X₁₅X₁₆ | |
| | RNDALTEHX₁₇X₁₈X₁₉HX₂₀X₂₁X₂₂X₂₃X₂₄X₈X₉CX₁₀X₁₁CX₁₂X₁₃X₁₄X₁₅ | |
| | X₁₆DKKDLTRHX₁₇X₁₈X₁₉HX₂₀X₂₁X₂₂X₂₃X₂₄X₂₅X₂₆X₂₇X₂₈X₂₉X₃₀ | |
| ZF3 | X₁X₂X₃X₄X₅X₆X₇X₈X₉CX₁₀X₁₁CX₁₂X₁₃X₁₄X₁₅X₁₆RSDNLVRHX₁₇X₁₈ | 315 |
| | X₁₉HX₂₀X₂₁X₂₂X₂₃X₂₄X₈X₉CX₁₀X₁₁CX₁₂X₁₃X₁₄X₁₅X₁₆QLAHLRAHX₁₇ | |
| | X₁₈X₁₉HX₂₀X₂₁X₂₂X₂₃X₂₄X₈X₉CX₁₀X₁₁CX₁₂X₁₃X₁₄X₁₅X₁₆DPGHLVR | |
| | HX₁₇X₁₈X₁₉HX₂₀X₂₁X₂₂X₂₃X₂₄X₈X₉CX₁₀X₁₁CX₁₂X₁₃X₁₄X₁₅X₁₆QSSNL | |
| | VRHX₁₇X₁₈X₁₉HX₂₀X₂₁X₂₂X₂₃X₂₄X₈X₉CX₁₀X₁₁CX₁₂X₁₃X₁₄X₁₅X₁₆RK | |
| | DNLKNHX₁₇X₁₈X₁₉HX₂₀X₂₁X₂₂X₂₃X₂₄X₈X₉CX₁₀X₁₁CX₁₂X₁₃X₁₄X₁₅X61 | |
| | QAGHLASHX₁₇X₁₈X₁₉HX₂₀X₂₁X₂₂X₂₃X₂₄X₈X₉CX₁₀X₁₁CX₁₂X₁₃X₁₄X₁₅ | |
| | X₁₆TSGSLVRHX₁₇X₁₈X₁₉HX₂₀X₂₁X₂₂X₂₃X₂₄X₂₅X₂₆X₂₇X₂₈X₂₉X₃₀ | |
| ZF4 | X₁X₂X₃X₄X₅X₆X₇X₈X₉CX₁₀X₁₁CX₁₂X₁₃X₁₄X₁₅X₁₆QRAHLERHX₁₇X₁₈ | 316 |
| | X₁₉HX₂₀X₂₁X₂₂X₂₃X₂₄X₈X₉CX₁₀X₁₁CX₁₂X₁₃X₁₄X₁₅X₁₆QLAHLRAHX₁₇ | |
| | X₁₈X₁₉HX₂₀X₂₁X₂₂X₂₃X₂₄X₈X₉CX₁₀X₁₁CX₁₂X₁₃X₁₄X₁₅X₁₆SKKHLAE | |
| | HX₁₇X₁₈X₁₉HX₂₀X₂₁X₂₂X₂₃X₂₄X₈X₉CX₁₀X₁₁CX₁₂X₁₃X₁₄X₁₅X₁₆TTGAL | |
| | TEHX₁₇X₁₈X₁₉HX₂₀X₂₁X₂₂X₂₃X₂₄X₈X₉CX₁₀X₁₁CX₁₂X₁₃X₁₄X₁₅X₁₆TSG | |
| | NLVRHX₁₇X₁₈X₁₉HX₂₀X₂₁X₂₂X₂₃X₂₄X₈X₉CX₁₀X₁₁CX₁₂X₁₃X₁₄X₁₅X₁₆ | |
| | QSGDLRRHX₁₇X₁₈X₁₉HX₂₀X₂₁X₂₂X₂₃X₂₄X₈X₉CX₁₀X₁₁CX₁₂X₁₃X₁₄X₁₅ | |
| | X₁₆TSHSLTEHX₁₇X₁₈X₁₉HX₂₀X₂₁X₂₂X₂₃X₂₄X₂₅X₂₆X₂₇X₂₈X₂₉X₃₀ | |
| ZF5 | X₁X₂X₃X₄X₅X₆X₇X₈X₉CX₁₀X₁₁CX₁₂X₁₃X₁₄X₁₅X₁₆TSGNLTEHX₁₇X₁₈ | 317 |
| | X₁₉HX₂₀X₂₁X₂₂X₂₃X₂₄X₈X₉CX₁₀X₁₁CX₁₂X₁₃X₁₄X₁₅X₁₆DSGNLRVHX₁₇ | |
| | X₁₈X₁₉HX₂₀X₂₁X₂₂X₂₃X₂₄X₈X₉CX₁₀X₁₁CX₁₂X₁₃X₁₄X₁₅X₁₆HKNALQN | |
| | HX₁₇X₁₈X₁₉HX₂₀X₂₁X₂₂X₂₃X₂₄X₈X₉CX₁₀X₁₁CX₁₂X₁₃X₁₄X₁₅X₁₆RNDT | |
| | LTEHX₁₇X₁₈X₁₉HX₂₀X₂₁X₂₂X₂₃X₂₄X₈X₉CX₁₀X₁₁CX₁₂X₁₃X₁₄X₁₅X₁₆QR | |
| | AHLERHX₁₇X₁₈X₁₉HX₂₀X₂₁X₂₂X₂₃X₂₄X₈X₉CX₁₀X₁₁CX₁₂X₁₃X₁₄X₁₅X₁₆ | |
| | RSDKLVRHX₁₇X₁₈X₁₉HX₂₀X₂₁X₂₂X₂₃X₂₄X₈X₉CX₁₀X₁₁CX₁₂X₁₃X₁₄X₁₅ | |
| | X₁₆DPGHLVRHX₁₇X₁₈X₁₉HX₂₀X₂₁X₂₂X₂₃X₂₄X₂₅X₂₆X₂₇X₂₈X₂₉X₃₀ | |
| ZF6 | X₁X₂X₃X₄X₅X₆X₇X₈X₉CX₁₀X₁₁CX₁₂X₁₃X₁₄X₁₅X₁₆TSHSLTEHX₁₇X₁₈ | 318 |
| | X₁₉HX₂₀X₂₁X₂₂X₂₃X₂₄X₈X₉CX₁₀X₁₁CX₁₂X₁₃X₁₄X₁₅X₁₆QRANLRAHX₁₇ | |
| | X₁₈X₁₉HX₂₀X₂₁X₂₂X₂₃X₂₄X₈X₉CX₁₀X₁₁CX₁₂X₁₃X₁₄X₁₅X₁₆RADNLTE | |
| | HX₁₇X₁₈X₁₉HX₂₀X₂₁X₂₂X₂₃X₂₄X₈X₉CX₁₀X₁₁CX₁₂X₁₃X₁₄X₁₅X₁₆ERSHL | |
| | REHX₁₇X₁₈X₁₉HX₂₀X₂₁X₂₂X₂₃X₂₄X₈X₉CX₁₀X₁₁CX₁₂X₁₃X₁₄X₁₅X₁₆DPG | |
| | ALVRHXI7X₁₈X₁₉HX₂₀X₂₁X₂₂X₂₃X₂₄X₈X₉CX₁₀X₁₁CX₁₂X₁₃X₁₄X₁₅X₁₆ | |
| | TSGHLVRHX₁₇X₁₈X₁₉HX₂₀X₂₁X₂₂X₂₃X₂₄X₈X₉CX₁₀X₁₁CX₁₂X₁₃X₁₄X₁₅ | |
| | X₁₆RNDTLTEHX₁₇X₁₈X₁₉HX₂₀X₂₁X₂₂X₂₃X₂₄X₂₅X₂₆X₂₇X₂₈X₂₉X₃₀ | |
| ZF7 | X₁X₂X₃X₄X₅X₆X₇X₈X₉CX₁₀X₁₁CX₁₂X₁₃X₁₄X₁₅X₁₆RSDHLTNHX₁₇X₁₈ | 319 |
| | X₁₉HX₂₀X₂₁X₂₂X₂₃X₂₄X₈X₉CX₁₀X₁₁CX₁₂X₁₃X₁₄X₁₅X₁₆TSGSLVRHX₁₇ | |
| | X₁₈X₁₉HX₂₀X₂₁X₂₂X₂₃X₂₄X₈X₉CX₁₀X₁₁CX₁₂X₁₃X₁₄X₁₅X₁₆QAGHLAS | |
| | HX₁₇X₁₈X₁₉HX₂₀X₂₁X₂₂X₂₃X₂₄X₈X₉CX₁₀X₁₁CX₁₂X₁₃X₁₄X₁₅X₁₆RSDKL | |
| | TEHX₁₇X₁₈X₁₉HX₂₀X₂₁X₂₂X₂₃X₂₄X₈X₉CX₁₀X₁₁CX₁₂X₁₃X₁₄X₁₅X₁₆TSG | |
| | NLTEHX₁₇X₁₈X₁₉HX₂₀X₂₁X₂₂X₂₃X₂₄X₈X₉CX₁₀X₁₁CX₁₂X₁₃X₁₄X₁₅X₁₆Q | |
| | SSNLVRHX₁₇X₁₈X₁₉HX₂₀X₂₁X₂₂X₂₃X₂₄X₈X₉CX₁₀X₁₁CX₁₂X₁₃X₁₄X₁₅X₁₆ | |
| | THLDLIRHX₁₇X₁₈X₁₉HX₂₀X₂₁X₂₂X₂₃X₂₄X₂₅X₂₆X₂₇X₂₈X₂₉X₃₀ | |
| ZF8 | X₁X₂X₃X₄X₅X₆X₇X₈X₉CX₁₀X₁₁CX₁₂X₁₃X₁₄X₁₅X₁₆RSDHLTTHX₁₇X₁₈ | 320 |
| | X₁₉HX₂₀X₂₁X₂₂X₂₃X₂₄X₈X₉CX₁₀X₁₁CX₁₂X₁₃X₁₄X₁₅X₁₆TSGELVRHX₁₇ | |
| | X₁₈X₁₉HX₂₀X₂₁X₂₂X₂₃X₂₄X₈X₉CX₁₀X₁₁CX₁₂X₁₃X₁₄X₁₅X₁₆RRDELNV | |
| | HX₁₇X₁₈X₁₉HX₂₀X₂₁X₂₂X₂₃X₂₄X₈X₉CX₁₀X₁₁CX₁₂X₁₃X₁₄X₁₅X₁₆SPADL | |
| | TRHX₁₇X₁₈X₁₉HX₂₀X₂₁X₂₂X₂₃X₂₄X₈X₉CX₁₀X₁₁CX₁₂X₁₃X₁₄X₁₅X₁₆RSD | |
| | ELVRHX₁₇X₁₈X₁₉HX₂₀X₂₁X₂₂X₂₃X₂₄X₈X₉CX₁₀X₁₁CX₁₂X₁₃X₁₄X₁₅X₁₆R | |
| | SDKLVRHX₁₇X₁₈X₁₉HX₂₀X₂₁X₂₂X₂₃X₂₄X₈X₉CX₁₀X₁₁CX₁₂X₁₃X₁₄X₁₅ | |
| | X₁₆TTGALTEHX₁₇X₁₈X₁₉HX₂₀X₂₁X₂₂X₂₃X₂₄X₂₅X₂₆X₂₇X₂₈X₂₉X₃₀ | |
| ZF9 | X₁X₂X₃X₄X₅X₆X₇X₈X₉CX₁₀X₁₁CX₁₂X₁₃X₁₄X₁₅X₁₆QSGNLTEHX₁₇X₁₈ | 321 |
| | X₁₉HX₂₀X₂₁X₂₂X₂₃X₂₄X₈X₉CX₁₀X₁₁CX₁₂X₁₃X₁₄X₁₅X₁₆HKNALQNHX₁₇ | |
| | X₁₈X₁₉HX₂₀X₂₁X₂₂X₂₃X₂₄X₈X₉CX₁₀X₁₁CX₁₂X₁₃X₁₄X₁₅X₁₆QNSTLTE | |
| | HX₁₇X₁₈X₁₉HX₂₀X₂₁X₂₂X₂₃X₂₄X₈X₉CX₁₀X₁₁CX₁₂X₁₃X₁₄X₁₅X₁₆SKKH | |
| | LAEHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | GNLVRHX₁₇X₁₈X₁₉HX₂₀X₂₁X₂₂X₂₃X₂₄X₈X₉CX₁₀X₁₁CX₁₂X₁₃X₁₄X₁₅X₁₆ | |
| | QSGHLTEHX₁₇X₁₈X₁₉HX₄₃X₄₄X₄₅X₄₆X₄₇X₄₈X₄₉X₅₀X₅₁X₅₂X₅₃X₅₄X₅₅ | |
| | X₅₆X₅₇X58X8X9CX₁₀X₁₁CX₁₂X₁₃X₁₄X₁₅X₁₆DKKDLTRHX₁₇X₁₈X₁₉HX₂₀ | |
| | X₂₁X₂₂X₂₃X₂₄X₂₅X₂₆X₂₇X₂₈X₂₉X₃₀ | |
| ZF10 | X₁X₂X₃X₄X₅X₆X₇X₈X₉CX₁₀X₁₁CX₁₂X₁₃X₁₄X₁₅X₁₆QNSTLTEHX₁₇X₁₈ | 322 |
| | X₁₉HX₂₀X₂₁X₂₂X₂₃X₂₄X₈X₉CX₁₀X₁₁CX₁₂X₁₃X₁₄X₁₅X₁₆ERSHLREHX₁₇ | |
| | X₁₈X₁₉HX₂₀X₂₁X₂₂X₂₃X₂₄X₈X₉CX₁₀X₁₁CX₁₂X₁₃X₁₄X₁₅X₁₆SKKHLAEH | |
| | X₁₇X₁₈X₁₉HX₂₀X₂₁X₂₂X₂₃X₂₄X₈X₉CX₁₀X₁₁CX₁₂X₁₃X₁₄X₁₅X₁₆RNDTLT | |
| | EHX₁₇X₁₈X₁₉HX₂₀X₂₁X₂₂X₂₃X₂₄X₈X₉CX₁₀X₁₁CX₁₂X₁₃X₁₄X₁₅X₁₆DCR | |
| | DLARHX₁₇X₁₈X₁₉HX₂₀X₂₁X₂₂X₂₃X₂₄X₈X₉CX₁₀X₁₁CX₁₂X₁₃X₁₄X₁₅X₁₆ | |
| | QSGDLRRHX₁₇X₁₈X₁₉HX₂₀X₂₁X₂₂X₂₃X₂₄X₈X₉CX₁₀X₁₁CX₁₂X₁₃X₁₄X₁₅ | |
| | X₁₆TKNSLTEHX₁₇X₁₈X₁₉HX₂₀X₂₁X₂₂X₂₃X₂₄X₂₅X₂₆X₂₇X₂₈X₂₉X₃₀ | |
| ZF11 | X₁X₂X₃X₄X₅X₆X₇X₈X₉CX₁₀X₁₁CX₁₂X₁₃X₁₄X₁₅X₁₆ERSHLREHX₁₇X₁₈ | 323 |
| | X₁₉HX₂₀X₂₁X₂₂X₂₃X₂₄X₈X₉CX₁₀X₁₁CX₁₂X₁₃X₁₄X₁₅X₁₆RADNLTEHX₁₇ | |
| | X₁₈X₁₉HX₂₀X₂₁X₂₂X₂₃X₂₄X₈X₉CX₁₀X₁₁CX₁₂X₁₃X₁₄X₁₅X₁₆RSDKLTEH | |
| | X₁₇X₁₈X₁₉HX₂₀X₂₁X₂₂X₂₃X₂₄X₈X₉CX₁₀X₁₁CX₁₂X₁₃X₁₄X₁₅X₁₆SKKHL | |
| | AEHX₁₇X₁₈X₁₉HX₂₀X₂₁X₂₂X₂₃X₂₄X₈X₉CX₁₀X₁₁CX₁₂X₁₃X₁₄X₁₅X₁₆TK | |
| | NSLTEHX₁₇X₁₈X₁₉HX₂₀X₂₁X₂₂X₂₃X₂₄X₈X₉CX₁₀X₁₁CX₁₂X₁₃X₁₄X₁₅X₁₆ | |
| | DKKDLTRHX₁₇X₁₈X₁₉HX₂₀X₂₁X₂₂X₂₃X₂₄X₈X₉CX₁₀X₁₁CX₁₂X₁₃X₁₄X₁₅ | |
| | X₁₆SKKHLAEHX₁₇X₁₈X₁₉HX₂₀X₂₁X₂₂X₂₃X₂₄X₂₅X₂₆X₂₇X₂₈X₂₉X₃₀ | |
| ZF12 | X₁X₂X₃X₄X₅X₆X₇X₈X₉CX₁₀X₁₁CX₁₂X₁₃X₁₄X₁₅X₁₆SKKHLAEHX₁₇X₁₈ | 324 |
| | X₁₉HX₂₀X₂₁X₂₂X₂₃X₂₄X₈X₉CX₁₀X₁₁CX₁₂X₁₃X₁₄X₁₅X₁₆THLDLIRHX₁₇ | |
| | X₁₈X₁₉HX₂₀X₂₁X₂₂X₂₃X₂₄X₈X₉CX₁₀X₁₁CX₁₂X₁₃X₁₄X₁₅X₁₆RKDNLKN | |
| | HX₁₇X₁₈X₁₉HX₂₀X₂₁X₂₂X₂₃X₂₄X₈X₉CX₁₀X₁₁CX₁₂X₁₃X₁₄X₁₅X₁₆DCRD | |
| | LARHX₁₇X₁₈X₁₉HX₂₀X₂₁X₂₂X₂₃X₂₄X₈X₉CX₁₀X₁₁CX₁₂X₁₃X₁₄X₁₅X₁₆RE | |
| | DNLHTHX₁₇X₁₈X₁₉HX₂₀X₂₁X₂₂X₂₃X₂₄X₈X₉CX₁₀X₁₁CX₁₂X₁₃X₁₄X₁₅X₁₆ | |
| | DPGHLVRHX₁₇X₁₈X₁₉HX₂₀X₂₁X₂₂X₂₃X₂₄X₈X₉CX₁₀X₁₁CX₁₂X₁₃X₁₄X₁₅ | |
| | X₁₆QLAHLRAHX₁₇X₁₈X₁₉HX₂₀X₂₁X₂₂X₂₃X₂₄X₂₅X₂₆X₂₇X₂₈X₂₉X₃₀ | |
| ZF13 | X₁X₂X₃X₄X₅X₆X₇X₈X₉CX₁₀X₁₁CX₁₂X₁₃X₁₄X₁₅X₁₆RSDKLVRHX₁₇X₁₈ | 325 |
| | X₁₉HX₂₀X₂₁X₂₂X₂₃X₂₄X₈X₉CX₁₀X₁₁CX₁₂X₁₃X₁₄X₁₅X₁₆QSGDLRRHX₁₇ | |
| | X₁₈X₁₉HX₂₀X₂₁X₂₂X₂₃X₂₄X₈X₉CX₁₀X₁₁CX₁₂X₁₃X₁₄X₁₅X₁₆TSGELVRH | |
| | X₁₇X₁₈X₁₉HX₂₀X₂₁X₂₂X₂₃X₂₄X₈X₉CX₁₀X₁₁CX₁₂X₁₃X₁₄X₁₅X₁₆RSDKLV | |
| | RHX₁₇X₁₈X₁₉HX₂₀X₂₁X₂₂X₂₃X₂₄X₈X₉CX₁₀X₁₁CX₁₂X₁₃X₁₄X₁₅X₁₆DPG | |
| | HLVRHX₁₇X₁₈X₁₉HX₂₀X₂₁X₂₂X₂₃X₂₄X₈X₉CX₁₀X₁₁CX₁₂X₁₃X₁₄X₁₅X₁₆ | |
| | RNDALTEHX₁₇X₁₈X₁₉HX₂₀X₂₁X₂₂X₂₃X₂₄X₈X₉CX₁₀X₁₁CX₁₂X₁₃X₁₄X₁₅ | |
| | X₁₆RSDHLTNHX₁₇X₁₈X₁₉HX₂₀X₂₁X₂₂X₂₃X₂₄X₂₅X₂₆X₂₇X₂₈X₂₉X₃₀ | |
| ZF14 | X₁X₂X₃X₄X₅X₆X₇X₈X₉CX₁₀X₁₁CX₁₂X₁₃X₁₄X₁₅X₁₆TSGHLVRHX₁₇X₁₈ | 326 |
| | X₁₉HX₂₀X₂₁X₂₂X₂₃X₂₄X₈X₉CX₁₀X₁₁CX₁₂X₁₃X₁₄X₁₅X₁₆TTGNLTVHX₁₇ | |
| | X₁₈X₁₉HX₂₀X₂₁X₂₂X₂₃X₂₄X₈X₉CX₁₀X₁₁CX₁₂X₁₃X₁₄X₁₅X₁₆TSGSLVRH | |
| | X₁₇X₁₈X₁₉HX₂₀X₂₁X₂₂X₂₃X₂₄X₈X₉CX₁₀X₁₁CX₁₂X₁₃X₁₄X₁₅X₁₆RSDKLV | |
| | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDE | |
| | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | DKLVRHX₁₇X₁₈X₁₉HX₂₀X₂₁X₂₂X₂₃X₂₄X₈X₉CX₁₀X₁₁CX₁₂X₁₃X₁₄X₁₅X₁₆ | |
| | QRAHLERHX₁₇X₁₈X₁₉HX₂₀X₂₁X₂₂X₂₃X₂₄X₂₅X₂₆X₂₇X₂₈X₂₉X₃₀ | |
| ZF15 | X₁X₂X₃X₄X₅X₆X₇X₈X₉CX₁₀X₁₁CX₁₂X₁₃X₁₄X₁₅X₁₆RNDTLTEHX₁₇X₁₈ | 327 |
| | X₁₉HX₂₀X₂₁X₂₂X₂₃X₂₄X₈X₉CX₁₀X₁₁CX₁₂X₁₃X₁₄X₁₅X₁₆RNDALTEHX₁₇ | |
| | X₁₈X₁₉HX₂₀X₂₁X₂₂X₂₃X₂₄X₈X₉CX₁₀X₁₁CX₁₂X₁₃X₁₄X₁₅X₁₆TSGELVRH | |
| | X₁₇X₁₈X₁₉HX₂₀X₂₁X₂₂X₂₃X₂₄X₈X₉CX₁₀X₁₁CX₁₂X₁₃X₁₄X₁₅X₁₆RSDNLV | |
| | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDE | |
| | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | DKLVRHX₁₇X₁₈X₁₉HX₂₀X₂₁X₂₂X₂₃X₂₄X₈X₉CX₁₀X₁₁CX₁₂X₁₃X₁₄X₁₅X₁₆ | |
| | QSSNLVRHX₁₇X₁₈X₁₉11X₂₀X₂₁X₂₂X₂₃X₂₄X₂₅X₂₆X₂₇X₂₈X₂₉X₃₀ | |

Similarly, the design and preparation of such TALE polypeptide which specifically bind to a DNA target region of interest, such as an HNF4α expression control region, is well known in the art. For example, the DNA binding domain of TALE contains a repeated highly conserved 33-34 amino acid sequence with divergent 12th and 13th amino acids. These two positions, referred to as the Repeat Variable Diresidue (RVD), are highly variable and show a strong correlation with specific nucleotide recognition. This straightforward relationship between amino acid sequence and DNA recognition has allowed for the engineering of specific DNA-binding domains by selecting a combination of repeat segments containing the appropriate RVDs. See, e.g., Boch J Nature Biotechnology. 29 (2) 2011: 135-6; Boch J, et al., Science. 326 (5959) 2009: 1509-12; Moscou M J & Bogdanove A J Science. 326 (5959) 2009: 1501.

In some embodiments, the site-specific HNF4α targeting moieties of the invention comprising a polynucleotide comprise a guide RNA (or gRNA) or nucleic acid encoding a guide RNA. A gRNA is a short synthetic RNA molecule comprising a “scaffold” sequence necessary for, e.g., directing an effector to an HNF4α expression control element which may, e.g., include an about 20 nucleotide site-specific sequence targeting a genomic target sequence comprising the HNF4α expression control element.

Generally, guide RNA sequences are designed to have a length of between about 17 to about 24 nucleotides (e.g., 19, 20, or 21 nucleotides) and are complementary to the target sequence. Custom gRNA generators and algorithms are available commercially for use in the design of effective guide RNAs. Gene editing has also been achieved using a chimeric “single guide RNA” (“sgRNA”), an engineered (synthetic) single RNA molecule that mimics a naturally occurring crRNA-tracrRNA complex and contains both a tracrRNA (for binding the nuclease) and at least one crRNA (to guide the nuclease to the sequence targeted for editing). Chemically modified sgNAs have also been demonstrated to be effective in genome editing; see, for example, Hendel et al. (2015) Nature Biotechnol., 985-991.

Exemplary site-specific HNF4α promoter 1 targeting moieties are provided in Table 2, below. In some embodiments, the polynucleotide comprises a nucleotide sequence at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the entire nucleotide sequence of any one of the nucleotide sequences in Table 2.

Exemplary site-specific HNF4α promoter 2 targeting moieties are provided in Table 3, below. In some embodiments, the polynucleotide comprises a nucleotide sequence at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the entire nucleotide sequence of any one of the nucleotide sequences in Table 3.

Exemplary site-specific HNF4α promoter targeting moieties are also provided in Table 9, below. In some embodiments, the polynucleotide comprises a nucleotide sequence at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the entire nucleotide sequence of any one of the nucleotide sequences in Table 9.

It will be understood that, although the sequences in Tables 2, 3, 4, and 9 are described as modified (or unmodified), the nucleic acid molecule encompassed by the of the invention, e.g., a site-specific disrupting agent, may comprise any one of the sequences set forth in any one of Tables 2, 3, 4, or 9 that is un-modified or modified differently than described therein. It will also be understood that although some of the sequences in Table 9 have “Ts”, when used as an RNA molecule, such as a guide RNA, in the site-specific targeting moieties of the invention, the “Ts” may be replaced with “Us.”

In some embodiments, a site-specific HNF4α targeting moiety comprising a polynucleotide, e.g., gRNA, comprises a nucleotide sequence complementary to an anchor sequence. In one embodiment, the anchor sequence comprises a CTCF-binding motif or consensus sequence: N(T/C/G)N(G/A/T)CC(A/T/G)(C/G)(C/T/A)AG(G/A)(G/T)GG(C/A/T)(G/A)(C/G)(C/T/A)(G/A/C) (SEQ ID NO: 64), where N is any nucleotide. A CTCF-binding motif or consensus sequence may also be in the opposite orientation, e.g., (G/A/C)(C/T/A)(C/G)(G/A)(C/A/T)GG(G/T)(G/A)GA(C/T/A)(C/G)(A/T/G)CC(G/A/T)N(T/C/G)N (SEQ ID NO: 65). In some embodiments, the nucleic acid sequence comprises a sequence complementary to a CTCF-binding motif or consensus sequence.

In some embodiments, the polynucleotide comprises a nucleotide sequence at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% complementary to an anchor sequence.

In some embodiments, the polynucleotide comprises a nucleotide sequence at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% complementary to a CTCF-binding motif or consensus sequence. In some embodiments, the polynucleotide is selected from the group consisting of a gRNA, and a sequence complementary or a sequence comprising at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% complementary sequence to an anchor sequence.

In some embodiments, a site-specific HNF4α targeting moiety comprising a polynucleotide of the invention is an RNAi molecule. RNAi molecules comprise RNA or RNA-like structures typically containing 15-50 base pairs (such as about 18-25 base pairs) and having a nucleobase sequence identical (complementary) or nearly identical (substantially complementary) to a coding sequence in an expressed target gene within the cell. RNAi molecules include, but are not limited to: short interfering RNAs (siRNAs), double-strand RNAs (dsRNA), micro RNAs (miRNAs), short hairpin RNAs (shRNA), meroduplexes, and dicer substrates (U.S. Pat. Nos. 8,084,599, 8,349,809, and 8,513,207). In one embodiment, the invention includes a composition to inhibit expression of a gene encoding a polypeptide described herein, e.g., a conjunction nucleating molecule.

RNAi molecules comprise a sequence substantially complementary, or fully complementary, to all or a fragment of a target gene. RNAi molecules may complement sequences at the boundary between introns and exons to prevent the maturation of newly-generated nuclear RNA transcripts of specific genes into mRNA for transcription. RNAi molecules complementary to specific genes can hybridize with the mRNA for that gene and prevent its translation. The antisense molecule can be DNA, RNA, or a derivative or hybrid thereof. Examples of such derivative molecules include, but are not limited to, peptide nucleic acid (PNA) and phosphorothioate-based molecules such as deoxyribonucleic guanidine (DNG) or ribonucleic guanidine (R G).

RNAi molecules can be provided to the cell as “ready-to-use” RNA synthesized in vitro or as an antisense gene transfected into cells which will yield RNAi molecules upon transcription. Hybridization with mRNA results in degradation of the hybridized molecule by RNAse H and/or inhibition of the formation of translation complexes. Both result in a failure to produce the product of the original gene.

The length of the RNAi molecule that hybridizes to the transcript of interest should be around 10 nucleotides, between about 15 or 30 nucleotides, or about 15, 16, 17, 18, 19, 20, 21, 22, 23, 24, 25, 26, 27, 28, 29, 30 or more nucleotides. The degree of identity of the antisense sequence to the targeted transcript should be at least 75%, at least 80%, at least 85%, at least 90%, or at least 95.

RNAi molecules may also comprise overhangs, i.e. typically unpaired, overhanging nucleotides which are not directly involved in the double helical structure normally formed by the core sequences of the herein defined pair of sense strand and antisense strand. RNAi molecules may contain 3′ and/or 5′ overhangs of about 1-5 bases independently on each of the sense strands and antisense strands. In one embodiment, both the sense strand and the antisense strand contain 3′ and 5′ overhangs. In one embodiment, one or more of the 3′ overhang nucleotides of one strand base pairs with one or more 5′ overhang nucleotides of the other strand. In another embodiment, the one or more of the 3′ overhang nucleotides of one strand base do not pair with the one or more 5′ overhang nucleotides of the other strand. The sense and antisense strands of an RNAi molecule may or may not contain the same number of nucleotide bases. The antisense and sense strands may form a duplex wherein the 5′ end only has a blunt end, the 3′ end only has a blunt end, both the 5′ and 3′ ends are blunt ended, or neither the 5′ end nor the 3′ end are blunt ended. In another embodiment, one or more of the nucleotides in the overhang contains a thiophosphate, phosphorothioate, deoxynucleotide inverted (3′ to 3′ linked) nucleotide or is a modified ribonucleotide or deoxynucleotide.

Small interfering RNA (siRNA) molecules comprise a nucleotide sequence that is identical to about 15 to about 25 contiguous nucleotides of the target mRNA. In some embodiments, the siRNA sequence commences with the dinucleotide AA, comprises a GC-content of about 30-70% (about 50-60%, about 40-60%, or about 45%-55%), and does not have a high percentage identity to any nucleotide sequence other than the target in the genome of the mammal in which it is to be introduced, for example as determined by standard BLAST search.

siRNAs and shRNAs resemble intermediates in the processing pathway of the endogenous microRNA (miRNA) genes (Bartel, Cell 116:281-297, 2004). In some embodiments, siRNAs can function as miRNAs and vice versa (Zeng et al., Mol Cell 9: 1327-1333, 2002; Doench et al., Genes Dev 17:438-442, 2003). MicroRNAs, like siRNAs, use RISC to downregulate target genes, but unlike siRNAs, most animal miRNAs do not cleave the mRNA. Instead, miRNAs reduce protein output through translational suppression or polyA removal and mRNA degradation (Wu et al., Proc Natl Acad Sci USA 103:4034-4039, 2006). Known miRNA binding sites are within mRNA 3′ UTRs; miRNAs seem to target sites with near-perfect complementarity to nucleotides 2-8 from the miRNA's 5′ end (Rajewsky, Nat Genet 38 Suppl: S8-13, 2006; Lim et al, Nature 433:769-773, 2005). This region is known as the seed region. Because siRNAs and miRNAs are interchangeable, exogenous siRNAs downregulate mRNAs with seed complementarity to the siRNA (Birmingham et al., Nat Methods 3: 199-204, 2006. Multiple target sites within a 3′ UTR give stronger downregulation (Doench et al., Genes Dev 17:438-442, 2003).

Lists of known miRNA sequences can be found in databases maintained by research organizations, such as Wellcome Trust Sanger Institute, Perm Center for Bioinformatics, Memorial Sloan Kettering Cancer Center, and European Molecule Biology Laboratory, among others. Known effective siRNA sequences and cognate binding sites are also well represented in the relevant literature. RNAi molecules are readily designed and produced by technologies known in the art. In addition, there are computational tools that increase the chance of finding effective and specific sequence motifs (Pei et al. 2006, Reynolds et al. 2004, Khvorova et al. 2003, Schwarz et al. 2003, Ui-Tei et al. 2004, Heale et al. 2005, Chalk et al. 2004, Amarzguioui et al. 2004).

An RNAi molecule modulates expression of RNA encoded by a gene. Because multiple genes can share some degree of sequence homology with each other, in some embodiments, the RNAi molecule can be designed to target a class of genes with sufficient sequence homology. In some embodiments, the RNAi molecule can contain a sequence that has complementarity to sequences that are shared amongst different gene targets or are unique for a specific gene target. In some embodiments, the RNAi molecule can be designed to target conserved regions of an RNA sequence having homology between several genes thereby targeting several genes in a gene family (e.g., different gene isoforms, splice variants, mutant genes, etc.). In some embodiments, the RNAi molecule can be designed to target a sequence that is unique to a specific RNA sequence of a single gene.

In some embodiments, the RNAi molecule targets a sequence in a conjunction nucleating molecule, e.g., CTCF, cohesin, USF 1, YY1, TATA-box binding protein associated factor 3 (TAF3), ZNF 143, or another polypeptide that promotes the formation of an anchor sequence-mediated conjunction, or an epigenetic modifying agent, e.g., an enzyme involved in post-translational modifications including, but are not limited to, DNA methylases (e.g., DNMT3a, DNMT3b, DNMTL), DNA demethylation (e.g., the TET family enzymes catalyze oxidation of 5-methylcytosine to 5-hydroxymethylcytosine and higher oxidative derivatives), histone methyltransferases, histone deacetylase (e.g., HDAC1, HDAC2, HDAC3), sirtuin 1, 2, 3, 4, 5, 6, or 7, lysine-specific histone demethylase 1 (LSD1), histone-lysine-N-methyltransferase (Setdbl), euchromatic histone-lysine N-methyltransferase 2 (G9a), histone-lysine N-methyltransferase (SUV39H1), enhancer of zeste homolog 2 (EZH2), viral lysine methyltransferase (vSET), histone methyltransferase (SET2), protein-lysine N-methyltransferase (SMYD2), and others. In one embodiment, the RNAi molecule targets a protein deacetylase, e.g., sirtuin 1, 2, 3, 4, 5, 6, or 7. In one embodiment, the invention includes a composition comprising an RNAi that targets a conjunction nucleating molecule, e.g., CTCF.

In some embodiments, the site-specific HNF4α targeting moiety comprises a peptide or protein moiety. In some embodiments, a site-specific disrupting agent comprises a fusion protein. In some embodiments, an effector is ca peptide or protein moiety. The peptide or protein moieties may include, but is not limited to, a peptide ligand, antibody fragment, or targeting aptamer that binds a receptor such as an extracellular receptor, neuropeptide, hormone peptide, peptide drug, toxic peptide, viral or microbial peptide, synthetic peptide, and agonist or antagonist peptide.

Exemplary peptides or protein include a DNA-binding protein, a CRISPR component protein, a conjunction nucleating molecule, a dominant negative conjunction nucleating molecule, an epigenetic modifying agent, or any combination thereof. In some embodiments, the peptide comprises a nuclease, a physical blocker, an epigenetic recruiter, and an epigenetic CpG modifier, and fragments and combinations of any of the foregoing. In some embodiments, the peptide comprises a DNA-binding domain of a protein, such as a helix-turn-helix motif, a leucine zipper, a Zn-finger, a TATA box binding proteins, a transcription factor.

Peptides or proteins may be linear or branched. The peptide or protein moiety may have a length from about 5 to about 200 amino acids, about 15 to about 150 amino acids, about 20 to about 125 amino acids, about 25 to about 100 amino acids, 20-70 amino acids, 20-80 amino acids, 20-90 amino acids, 30-100 amino acids, 30-60 amino acids, 30-80 amino acids, 35-85 amino acids, 40-100 amino acids, or 50-125 amino acids or any range therebetween.

As indicated above, in some embodiments, the site-specific HNF4α targeting moieties of the invention comprise a fusion protein.

In some embodiments, the fusion proteins of the invention include a site-specific HNF4α targeting moiety which targets an HNF4α expression control region and an effector molecule. In other embodiments, a fusion protein of the invention comprises an effector molecule. Exemplary effector molecules include are described below and in some embodiments include, for example, nucleases, physical blockers, epigenetic recruiters, e.g., a transcriptional enhancer or a transcriptional repressor, and epigenetic CpG modifiers, e.g., a DNA methylase, a DNA demethylase, a histone modifying agent, or a histone deacetylase, and combinations of any of the foregoing.

For example, a site-specific targeting moiety may comprise a gRNA and an effector, such as a nuclease, e.g., a Cas9, e.g., a wild type Cas9, a nickase Cas9 (e.g., Cas9 D10A), a dead Cas9 (dCas9), eSpCas9, Cpf1, C2C1, or C2C3, or a nucleic acid encoding such a nuclease. The choice of nuclease and gRNA(s) is determined by whether the targeted mutation is a deletion, substitution, or addition of nucleotides, e.g., a deletion, substitution, or addition of nucleotides to a targeted sequence. Fusions of a catalytically inactive endonuclease e.g., a dead Cas9 (dCas9, e.g., D10A; H840A) tethered with all or a portion of (e.g., biologically active portion of) an (one or more) effector domain create chimeric proteins that can be linked to the polypeptide to guide the composition to specific DNA sites by one or more RNA sequences (e.g., DNA recognition elements including, but not restricted to zinc finger arrays, sgRNA, TAL arrays, peptide nucleic acids described herein) to modulate activity and/or expression of one or more target nucleic acids sequences (e.g., to methylate or demethylate a DNA sequence).

In one embodiment, a fusion protein of the invention may comprise an effector molecule comprising, for example, a CRISPR associated protein (Cas) polypeptide, or fragment thereof, (e.g., a Cas9 polypeptide, or fragment thereof) and an epigenetic recruiter or an epigenetic CpG modifier.

In one embodiment, a suitable Cas polypeptide is an enzymatically inactive Cas polypeptide, e.g., a “dead Cas polypeptide” or “dCas” polypeptide

Exemplary Cas polypeptides that are adaptable to the methods and compositions described herein are described below. Using methods known in the art, a Cas polypeptide can be fused to any of a variety of agents and/or molecules as described herein; such resulting fusion molecules can be useful in various disclosed methods.

In one aspect, the invention includes a composition comprising a protein comprising a domain, e.g., an effector, that acts on DNA (e.g., a nuclease domain, e.g., a Cas9 domain, e.g., a dCas9 domain; a DNA methyltransferase, a demethylase, a deaminase), in combination with at least one guide RNA (gRNA) or antisense DNA oligonucleotide that targets the protein to site-specific target sequence, wherein the composition is effective to alter, in a human cell, the expression of a target gene. In some embodiments, the enzyme domain is a Cas9 or a dCas9. In some embodiments, the protein comprises two enzyme domains, e.g., a dCas9 and a methylase or demethylase domain.

In one aspect, the invention includes a composition comprising a protein comprising a domain, e.g., an effector, that comprises a transcriptional control element (e.g., a nuclease domain, e.g., a Cas9 domain, e.g., a dCas9 domain; a transcriptional enhancer; a transcriptional repressor), in combination with at least one guide RNA (gRNA) or antisense DNA oligonucleotide that targets the protein to a site-specific target sequence, wherein the composition is effective to alter, in a human cell, the expression of a target gene. In some embodiments, the enzyme domain is a Cas9 or a dCas9. In some embodiments, the protein comprises two enzyme domains, e.g., a dCas9 and a transcriptional enhancer or transcriptional repressor domain.

As used herein, a “biologically active portion of an effector domain” is a portion that maintains the function (e.g. completely, partially, minimally) of an effector domain (e.g., a “minimal” or “core” domain).

The chimeric proteins described herein may also comprise a linker, e.g., an amino acid linker. In some aspects, a linker comprises 2 or more amino acids, e.g., one or more GS sequences. In some aspects, fusion of Cas9 (e.g., dCas9) with two or more effector domains (e.g., of a DNA methylase or enzyme with a role in DNA demethylation or protein acetyl transferase or deacetylase) comprises one or more interspersed linkers (e.g., GS linkers) between the domains. In some aspects, dCas9 is fused with 2-5 effector domains with interspersed linkers.

In some embodiments, a site-specific HNF4α targeting moiety comprises a conjunction nucleating molecule, a nucleic acid encoding a conjunction nucleating molecule, or a combination thereof. In some embodiments, an anchor sequence-mediated conjunction is mediated by a first conjunction nucleating molecule bound to the first anchor sequence, a second conjunction nucleating molecule bound to the noncontiguous second anchor sequence, and an association between the first and second conjunction nucleating molecules. In some embodiments, a conjunction nucleating molecule may disrupt, e.g., by competitive binding, the binding of an endogenous conjunction nucleating molecule to its binding site.

The conjunction nucleating molecule may be, e.g., CTCF, cohesin, USF1, YY1, TATA-box binding protein associated factor 3 (TAF3), ZNF143 binding motif, or another polypeptide that promotes the formation of an anchor sequence-mediated conjunction. The conjunction nucleating molecule may be an endogenous polypeptide or other protein, such as a transcription factor, e.g., autoimmune regulator (AIRE), another factor, e.g., X-inactivation specific transcript (XIST), or an engineered polypeptide that is engineered to recognize a specific DNA sequence of interest, e.g., having a zinc finger, leucine zipper or bHLH domain for sequence recognition. The conjunction nucleating molecule may modulate DNA interactions within or around the anchor sequence-mediated conjunction. For example, the conjunction nucleating molecule can recruit other factors to the anchor sequence that alters an anchor sequence-mediated conjunction formation or disruption.

The conjunction nucleating molecule may also have a dimerization domain for homo- or heterodimerization. One or more conjunction nucleating molecules, e.g., endogenous and engineered, may interact to form the anchor sequence-mediated conjunction. In some embodiments, the conjunction nucleating molecule is engineered to further include a stabilization domain, e.g., cohesion interaction domain, to stabilize the anchor sequence-mediated conjunction. In some embodiments, the conjunction nucleating molecule is engineered to bind a target sequence, e.g., target sequence binding affinity is modulated. In some embodiments, the conjunction nucleating molecule is selected or engineered with a selected binding affinity for an anchor sequence within the anchor sequence-mediated conjunction. Conjunction nucleating molecules and their corresponding anchor sequences may be identified through the use of cells that harbor inactivating mutations in CTCF and Chromosome Conformation Capture or 3C-based methods, e.g., Hi-C or high-throughput sequencing, to examine topologically associated domains, e.g., topological interactions between distal DNA regions or loci, in the absence of CTCF. Long-range DNA interactions may also be identified. Additional analyses may include ChlA-PET analysis using a bait, such as Cohesin, YY1 or USF1, ZNF143 binding motif, and MS to identify complexes that are associated with the bait.

### B. Effector Molecules

Effector molecules for use in the compositions and methods of the invention include those that modulate a biological activity, for example increasing or decreasing enzymatic activity, gene expression, cell signalling, and cellular or organ function. Preferred effector molecules of the invention are nucleases, physical blockers, epigenetic recruiters, e.g., a transcriptional enhancer or a transcriptional repressor, and epigenetic CpG modifiers, e.g., a DNA methylase, a DNA demethylase, a histone modifying agent, or a histone deacetylase, and combinations of any of the foregoing.

Additional effector activities of the effector molecules of the invention may also include binding regulatory proteins to modulate activity of the regulator, such as transcription or translation. Effector molecules also may include activator or inhibitor (or “negative effector”) functions as described herein. For example, the effector molecule may inhibit substrate binding to a receptor and inhibit its activation, e.g., naltrexone and naloxone bind opioid receptors without activating them and block the receptors' ability to bind opioids. Effector molecules may also modulate protein stability/degradation and/or transcript stability/degradation. For example, proteins may be targeted for degradation by the polypeptide co-factor, ubiquitin, onto proteins to mark them for degradation. In another example, an effector molecule inhibits enzymatic activity by blocking the enzyme's active site, e.g., methotrexate is a structural analog of tetrahydrofolate, a coenzyme for the enzyme dihydrofolate reductase that binds to dihydrofolate reductase 1000-fold more tightly than the natural substrate and inhibits nucleotide base synthesis.

In some embodiments, the effector molecule is a chemical, e.g., a chemical that modulates a cytosine (C) or an adenine (A) (e.g., Na bisulfite, ammonium bisulfite). In some embodiments, the effector molecule has enzymatic activity (methyl transferase, demethylase, nuclease (e.g., Cas9), a deaminase). In some embodiments, the effector molecule sterically hinders formation of an anchor sequence-mediated conjunction or binding of an RNA polymerase to a promoter.

The effector molecule with effector activity may be any one of the small molecules, peptides, fusion proteins, nucleic acids, nanoparticle, aptamers, or pharmacoagents with poor PK/PD described herein.

In some embodiments, the effector molecule is an inhibitor or “negative effector molecule”. In the context of a negative effector molecule that modulates formation of an anchor sequence-mediated conjunction, in some embodiments, the negative effector molecule is characterized in that dimerization of an endogenous nucleating polypeptide is reduced when the negative effector molecule is present as compared with when it is absent. For example, in some embodiments, the negative effector molecule is or comprises a variant of the endogenous nucleating polypeptide's dimerization domain, or a dimerizing portion thereof.

For example, in certain embodiments, an anchor sequence-mediated conjunction is altered (e.g., disrupted) by use of a dominant negative effector, e.g., a protein that recognizes and binds an anchor sequence, (e.g., a CTCF binding motif), but with an inactive (e.g., mutated) dimerization domain, e.g., a dimerization domain that is unable to form a functional anchor sequence-mediated conjunction. For example, the Zinc Finger domain of CTCF can be altered so that it binds a specific anchor sequence (by adding zinc fingers that recognize flanking nucleic acids), while the homo-dimerization domain is altered to prevent the interaction between the engineered CTCF and endogenous forms of CTCF.

In some embodiments, the effector molecule comprises a synthetic conjunction nucleating molecule with a selected binding affinity for an anchor sequence within a target anchor sequence-mediated conjunction, (the binding affinity may be at least 10%, 15%, 20%, 25%, 30%, 35%, 40%, 45%, 50%, 55%, 60%, 65%, 70%, 75%, 80%, 85%, 90%, 95%, or higher or lower than the affinity of an endogenous conjunction nucleating molecule that associates with the target anchor sequence. The synthetic conjunction nucleating molecule may have between 30-90%, 30-85%, 30-80%, 30-70%, 50-80%, 50-90% amino acid sequence identity to the endogenous conjunction nucleating molecule). The conjunction nucleating molecule may disrupt, such as through competitive binding, the binding of an endogenous conjunction nucleating molecule to its anchor sequence. In some more embodiments, the conjunction nucleating molecule is engineered to bind a novel anchor sequence within the anchor sequence-mediated conjunction.

In some embodiments, a dominant negative effector molecule has a domain that recognizes specific DNA sequences (e.g., an anchor sequence, a CTCF anchor sequence, flanked by sequences that confer sequence specificity), and a second domain that provides a steric presence in the vicinity of the anchoring sequence. The second domain may include a dominant negative conjunction nucleating molecule or fragment thereof, a polypeptide that interferes with conjunction nucleating molecule sequence recognition (e.g., the amino acid backbone of a peptide/nucleic acid or PNA), a nucleic acid sequence ligated to a small molecule that imparts steric interference, or any other combination of DNA recognition elements and a steric blocker.

In some embodiments, the effector molecule is an epigenetic modifying agent. Epigenetic modifying agents useful in the methods and compositions described herein include agents that affect, e.g., DNA methylation, histone acetylation, and RNA-associated silencing. In some embodiments, the effectors sequence-specifically target an epigenetic enzyme (e.g., an enzyme that generates or removes epigenetic marks, e.g., acetylation and/or methylation). Exemplary epigenetic effectors may target an expression control region comprising, e.g., a transcriptional control element or an anchor sequence, by a site-specific disrupting agent comprising a site-specific targeting moiety.

In some embodiments, an effector molecule comprises one or more components of a gene editing system. Components of gene editing systems may be used in a variety of contexts including but not limited to gene editing. For example, such components may be used to target agents that physically modify, genetically modify, and/or epigenetically modify HNF4α sequences.

Exemplary gene editing systems include the clustered regulatory interspaced short palindromic repeat (CRISPR) system, zinc finger nucleases (ZFNs), and Transcription Activator-Like Effector-based Nucleases (TALEN). ZFNs, TALENs, and CRISPR-based methods are described, e.g., in Gaj et al. Trends Biotechnol. 31.7(2013):397-405; CRISPR methods of gene editing are described, e.g., in Guan et al, Application of CRISPR-Cas system in gene therapy: Pre-clinical progress in animal model. DNA Repair 2016 Jul. 30 [Epub ahead of print]; Zheng et al, Precise gene deletion and replacement using the CRISPR/Cas9 system in human cells. BioTechniques, Vol. 57, No. 3, September 2014, pp. 115-124.

CRISPR systems are adaptive defense systems originally discovered in bacteria and archaea. CRISPR systems use RNA-guided nucleases termed CRISPR-associated or “Cas” endonucleases (e. g., Cas9 or Cpf1) to cleave foreign DNA. In a typical CRISPR/Cas system, an endonuclease is directed to a target nucleotide sequence (e. g., a site in the genome that is to be sequence-edited) by sequence-specific, non-coding “guide RNAs” that target single- or double-stranded DNA sequences. Three classes (I-III) of CRISPR systems have been identified. The class II CRISPR systems use a single Cas endonuclease (rather than multiple Cas proteins). One class II CRISPR system includes a type II Cas endonuclease such as Cas9, a CRISPR RNA (“crRNA”), and a trans-activating crRNA (“tracrRNA”). The crRNA contains a “guide RNA”, typically about 20-nucleotide RNA sequence that corresponds to a target DNA sequence. The crRNA also contains a region that binds to the tracrRNA to form a partially double-stranded structure which is cleaved by RNase III, resulting in a crRNA/tracrRNA hybrid. The crRNA/tracrRNA hybrid then directs the Cas9 endonuclease to recognize and cleave the target DNA sequence. The target DNA sequence must generally be adjacent to a “protospacer adjacent motif (“PAM”) that is specific for a given Cas endonuclease; however, PAM sequences appear throughout a given genome. CRISPR endonucleases identified from various prokaryotic species have unique PAM sequence requirements; examples of PAM sequences include 5′-NGG (Streptococcus pyogenes), 5′-NNAGAA (Streptococcus thermophilus CRISPR1), 5′-NGGNG (Streptococcus thermophilus CRISPR3), and 5′-NNNGATT (Neisseria meningiditis). Some endonucleases, e. g., Cas9 endonucleases, are associated with G-rich PAM sites, e. g., 5′-NGG, and perform blunt-end cleaving of the target DNA at a location 3 nucleotides upstream from (5′ from) the PAM site. Another class II CRISPR system includes the type V endonuclease Cpf1, which is smaller than Cas9; examples include AsCpf1 (from Acidaminococcus sp.) and LbCpf1 (from Lachnospiraceae sp.). Cpf 1-associated CRISPR arrays are processed into mature crRNAs without the requirement of a tracrRNA; in other words a Cpf1 system requires only the Cpf1 nuclease and a crRNA to cleave the target DNA sequence. Cpf1 endonucleases, are associated with T-rich PAM sites, e. g., 5′-TTN. Cpf1 can also recognize a 5′-CTA PAM motif. Cpf1 cleaves the target DNA by introducing an offset or staggered double-strand break with a 4- or 5-nucleotide 5′ overhang, for example, cleaving a target DNA with a 5-nucleotide offset or staggered cut located 18 nucleotides downstream from (3′ from) from the PAM site on the coding strand and 23 nucleotides downstream from the PAM site on the complimentary strand; the 5-nucleotide overhang that results from such offset cleavage allows more precise genome editing by DNA insertion by homologous recombination than by insertion at blunt-end cleaved DNA. See, e. g., Zetsche et al. (2015) Cell, 163:759-771.

A variety of CRISPR associated (Cas) genes or proteins can be used in the present invention and the choice of Cas protein will depend upon the particular conditions of the method.

Specific examples of Cas proteins include class II systems including Cas1, Cas2, Cas3, Cas4, Cas5, Cas6, Cas7, Cas8, Cas9, Cas10, Cpf1, C2C1, or C2C3. In some embodiments, a Cas protein, e.g., a Cas9 protein, may be from any of a variety of prokaryotic species. In some embodiments a particular Cas protein, e.g., a particular Cas9 protein, is selected to recognize a particular protospacer-adjacent motif (PAM) sequence. In some embodiments, the site-specific targeting moiety includes a sequence targeting polypeptide, such as an enzyme, e.g., Cas9. In certain embodiments a Cas protein, e.g., a Cas9 protein, may be obtained from a bacteria or archaea or synthesized using known methods.

In certain embodiments, a Cas protein may be from a gram positive bacteria or a gram negative bacteria. In certain embodiments, a Cas protein may be from a Streptococcus, (e.g., a S. pyogenes, a S. thermophilus) a Cryptococcus, a Corynebacterium, a Haemophilus, a Eubacterium, a Pasteurella, a Prevotella, a Veillonella, or a Marinobacter. In some embodiments nucleic acids encoding two or more different Cas proteins, or two or more Cas proteins, may be introduced into a cell, zygote, embryo, or animal, e.g., to allow for recognition and modification of sites comprising the same, similar or different PAM motifs. In some embodiments, the Cas protein is modified to deactivate the nuclease, e.g., nuclease-deficient Cas9, and to recruit transcription activators or repressors, e.g., the co-subunit of the E. coli Pol, VP64, the activation domain of p65, KRAB, or SID4X, to induce epigenetic modifications, e.g., histone acetyltransferase, histone methyltransferase and demethylase, DNA methyltransferase and enzyme with a role in DNA demethylation (e.g., the TET family enzymes catalyze oxidation of 5-methylcytosine to 5-hydroxymethylcytosine and higher oxidative derivatives).

For the purposes of gene editing, CRISPR arrays can be designed to contain one or multiple guide RNA sequences corresponding to a desired target DNA sequence; see, for example, Cong et al. (2013) Science, 339:819-823; Ran et al. (2013) Nature Protocols, 8:2281-2308. At least about 16 or 17 nucleotides of gRNA sequence are required by Cas9 for DNA cleavage to occur; for Cpf1 at least about 16 nucleotides of gRNA sequence is needed to achieve detectable DNA cleavage.

Whereas wild-type Cas9 generates double-strand breaks (DSBs) at specific DNA sequences targeted by a gRNA, a number of CRISPR endonucleases having modified functionalities are available, for example: a “nickase” version of Cas9 generates only a single-strand break; a catalytically inactive Cas9 (“dCas9”) does not cut the target DNA but interferes with transcription by steric hindrance. dCas9 can further be fused with a heterologous effector to repress (CRISPRi) or activate (CRISPRa) expression of a target gene. For example, Cas9 can be fused to a transcriptional silencer (e.g., a KRAB domain) or a transcriptional activator (e.g., a dCas9-VP64 fusion). A catalytically inactive Cas9 (dCas9) fused to FokI nuclease (“dCas9-FokI”) can be used to generate DSBs at target sequences homologous to two gRNAs. See, e. g., the numerous CRISPR/Cas9 plasmids disclosed in and publicly available from the Addgene repository (Addgene, 75 Sidney St., Suite 550A, Cambridge, MA 02139; addgene.org/crispr). A “double nickase” Cas9 that introduces two separate double-strand breaks, each directed by a separate guide RNA, is described as achieving more accurate genome editing by Ran et al. (2013) Cell, 154: 1380-1389.

CRISPR technology for editing the genes of eukaryotes is disclosed in US Patent Application Publications 2016/0138008A1 and US2015/0344912A1, and in U.S. Pat. Nos. 8,697,359, 8,771,945, 8,945,839, 8,999,641, 8,993,233, 8,895,308, 8,865,406, 8,889,418, 8,871,445, 8,889,356, 8,932,814, 8,795,965, and 8,906,616. Cpf1 endonuclease and corresponding guide RNAs and PAM sites are disclosed in US Patent Application Publication 2016/0208243 A1.

In some embodiments, an effector comprises one or more components of a CRISPR system described hereinabove.

In some embodiments, suitable effectors for use in the agents, compositions, and methods of the invention include, for example, nucleases, physical blockers, epigenetic recruiters, e.g., a transcriptional enhancer or a transcriptional repressor, and epigenetic CpG modifiers, e.g., a DNA methylase, a DNA demethylase, a histone modifying agent, or a histone deacetylase, and combinations of any of the foregoing.

Suitable effectors include a polypeptide or its variant. The term “variant,” as used herein, refers to a polypeptide that is derived by incorporation of one or more amino acid insertions, substitutions, or deletions in a precursor polypeptide (e.g., “parent” polypeptide). In certain embodiments, a variant polypeptide has at least about 85% amino acid sequence identity, e.g., about 90%, about 91%, about 92%, about 93%, about 94%, about 95%, about 96%, about 97%, about 98%, about 99%, or about 100%, amino acid sequence identity to the entire amino acid sequence of a parent polypeptide.

The term “sequence identity,” as used herein, refers to a comparison between pairs of nucleic acid or amino acid molecules, i.e., the relatedness between two amino acid sequences or between two nucleotide sequences. In general, the sequences are aligned so that the highest order match is obtained. Methods for determining sequence identity are known and can be determined by commercially available computer programs that can calculate the percentage of identity between two or more sequences. A typical example of such a computer program is CLUSTAL.

Exemplary effectors include ubiquitin, bicyclic peptides as ubiquitin ligase inhibitors, transcription factors, DNA and protein modification enzymes such as topoisomerases, topoisomerase inhibitors such as topotecan, DNA methyltransferases such as the DNMT family (e.g., DNMT3a, DNMT3b, DNMTL), protein methyltransferases (e.g., viral lysine methyltransferase (vSET), protein-lysine N-methyltransferase (SMYD2), deaminases (e.g., APOBEC, UG1), histone methyltransferases such as enhancer of zeste homolog 2 (EZH2), PRMT1, histone-lysine-N-methyltransferase (Setdbl), histone methyltransferase (SET2), euchromatic histone-lysine N-methyltransferase 2 (G9a), histone-lysine N-methyltransferase (SUV39H1), and G9a), histone deacetylase (e.g., HDAC1, HDAC2, HDAC3), enzymes with a role in DNA demethylation (e.g., the TET family enzymes catalyze oxidation of 5-methylcytosine to 5-hydroxymethylcytosine and higher oxidative derivatives), protein demethylases such as KDMIA and lysine-specific histone demethylase 1 (LSD1), helicases such as DHX9, acetyltransferases, deacetylases (e.g., sirtuin 1, 2, 3, 4, 5, 6, or 7), kinases, phosphatases, DNA-intercalating agents such as ethidium bromide, sybr green, and proflavine, efflux pump inhibitors such as peptidomimetics like phenylalanine arginyl-naphthylamide or quinoline derivatives, nuclear receptor activators and inhibitors, proteasome inhibitors, competitive inhibitors for enzymes such as those involved in lysosomal storage diseases, zinc finger proteins, TALENs, specific domains from proteins, such as a KRAB domain, a VP64 domain, a p300 domain (e.g., p300 core domain), an MeCP2 domain, an MQ1 domain, a DNMT3a-3L domain a TET1 domain, and a TET2 domain, protein synthesis inhibitors, nucleases (e.g., Cpf1, Cas9, zinc finger nuclease), fusions of one or more thereof (e.g., dCas9-DNMT, dCas9-APOBEC, dCas9-UG1, dCas9-VP64, dCas9-p300 core, dCas9-KRAB, dCas9-KRAB-MeCP2, dCas9-MQ1, dCas9-DNMT3a-3L, dCAS9-TET1, dCAS9-TET2, and dCas9-MC/MN).

In some embodiments, a suitable nuclease for use in the agent, compositions, and methods of the invention comprises a Cas9 polypeptide, or enzymatically active portion thereof. In one embodiment, the Cas9 polypeptide, or enzymatically active portion thereof, further comprises a catalytically active domain of human exonuclease 1 (hEXO1), e.g., 5′ to 3′ exonuclease activity and/or an RNase H activity. In other embodiments, a suitable nuclease comprises a transcription activator like effector nucleases (TALEN). In yet other embodiments, a suitable nuclease comprises a zinc finger protein.

The term TALEN, as used herein, is broad and includes a monomeric TALEN that can cleave double stranded DNA without assistance from another TALEN. The term TALEN is also used to refer to one or both members of a pair of TALENs that are engineered to work together to cleave DNA at the same site. TALENs that work together may be referred to as a left-TALEN and a right-TALEN, which references the handedness of DNA. See U.S. Ser. No. 12/965,590; U.S. Ser. No. 13/426,991 (U.S. Pat. No. 8,450,471); U.S. Ser. No. 13/427,040 (U.S. Pat. No. 8,440,431); U.S. Ser. No. 13/427,137 (U.S. Pat. No. 8,440,432); and U.S. Ser. No. 13/738,381, all of which are incorporated by reference herein in their entirety.

TAL effectors are proteins secreted by Xanthomonas bacteria. The DNA binding domain contains a highly conserved 33-34 amino acid sequence with the exception of the 12th and 13th amino acids. These two locations are highly variable (Repeat Variable Diresidue (RVD)) and show a strong correlation with specific nucleotide recognition. This simple relationship between amino acid sequence and DNA recognition has allowed for the engineering of specific DNA binding domains by selecting a combination of repeat segments containing the appropriate RVDs.

The non-specific DNA cleavage domain from the end of the FokI endonuclease can be used to construct hybrid nucleases that are active in a yeast assay. These reagents are also active in plant cells and in animal cells. Initial TALEN studies used the wild-type FokI cleavage domain, but some subsequent TALEN studies also used FokI cleavage domain variants with mutations designed to improve cleavage specificity and cleavage activity. The FokI domain functions as a dimer, requiring two constructs with unique DNA binding domains for sites in the target genome with proper orientation and spacing. Both the number of amino acid residues between the TALEN DNA binding domain and the FokI cleavage domain and the number of bases between the two individual TALEN binding sites are parameters for achieving high levels of activity. The number of amino acid residues between the TALEN DNA binding domain and the FokI cleavage domain may be modified by introduction of a spacer (distinct from the spacer sequence) between the plurality of TAL effector repeat sequences and the FokI endonuclease domain. The spacer sequence may be 12 to 30 nucleotides.

The relationship between amino acid sequence and DNA recognition of the TALEN binding domain allows for designable proteins. In this case artificial gene synthesis is problematic because of improper annealing of the repetitive sequence found in the TALE binding domain. One solution to this is to use a publicly available software program (DNAWorks) to calculate oligonucleotides suitable for assembly in a two step PCR; oligonucleotide assembly followed by whole gene amplification. A number of modular assembly schemes for generating engineered TALE constructs have also been reported. Both methods offer a systematic approach to engineering DNA binding domains that is conceptually similar to the modular assembly method for generating zinc finger DNA recognition domains.

Once the TALEN genes have been assembled they are inserted into plasmids; the plasmids are then used to transfect the target cell where the gene products are expressed and enter the nucleus to access the genome. TALENs can be used to edit genomes by inducing double-strand breaks (DSB), which cells respond to with repair mechanisms. In this manner, they can be used to correct mutations in the genome which, for example, cause disease.

As used herein, a “zinc finger polypeptide” or “zinc finger protein” is a protein that binds to DNA, RNA and/or protein, in a sequence-specific manner, by virtue of a metal stabilized domain known as a zinc finger. Zinc finger proteins are nucleases having a DNA cleavage domain and a DNA binding zinc finger domain. Zinc finger polypeptides may be made by fusing the nonspecific DNA. cleavage domain of an endonuclease with site-specific DNA binding zinc finger domains. Such nucleases are powerful tools for gene editing and can be assembled to induce double strand breaks (DSBs) site-specifically into genomic DNA. ZFNs allow specific gene disruption as during DNA repair, the targeted genes can be disrupted via mutagenic non-homologous end joint (NHEJ) or modified via homologous recombination (HR) if a closely related DNA template is supplied.

Zinc finger nucleases are chimeric enzymes made by fusing the nonspecific DNA. cleavage domain of the endonuclease FokI with site-specific DNA binding zinc finger domains. Due to the flexible nature of zinc finger proteins (ZFPs), ZFNs can be assembled that induce double strand breaks (DSBs) site-specifically into genomic DNA. ZFNs allow specific gene disruption as during DNA repair, the targeted genes can be disrupted via mutagenic non-homologous end joint (NHEJ) or modified via homologous recombination (HR) if a closely related DNA template is supplied.

In some embodiments, a suitable physical blocker for use in the agent, compositions, and methods of the invention comprises a gRNA, antisense DNA, or triplex forming oligonucleotide (which may target an expression control unit) steric block a transcriptional control element or anchoring sequence. The gRNA recognizes specific DNA sequences and further includes sequences that interfere with, e.g., a conjunction nucleating molecule sequence to act as a steric blocker. In some embodiments, the gRNA is combined with one or more peptides, e.g., S-adenosyl methionine (SAM), that acts as a steric presence. In other embodiments, a physical blocker comprises an enzymatically inactive Cas9 polypeptide, or fragment thereof (e.g., dCas9).

In one embodiment, an epigenetic recruiter activates or enhances transcription of a target gene. In some embodiments, a suitable epigenetic recruiter for use in the agent, compositions, and methods of the invention comprises a VP64 domain or a p300 core domain.

In one embodiment, an epigenetic recruiter silences or represses transcription of a target gene. In some embodiments, a suitable epigenetic recruiter for use in the agent, compositions, and methods of the invention comprises a KRAB domain, or an MeCP2 domain.

In one embodiment, a suitable epigenetic recruiter for use in the agent, compositions, and methods of the invention comprises dCas9-VP64 fusion, a dCas9-p300 core fusion, a dCas9-KRAB fusion, or a dCas9-KRAB-MeCP2 fusion.

As used herein, “VP64” is a transcriptional activator composed of four tandem copies of VP16 (Herpes Simplex Viral Protein 16, amino acids 437-447*: DALDDFDLDML (SEQ ID NO: 328)) connected with glycine-serine (GS) linkers. In one embodiment, the VP64 further comprises the transcription factors p65 (RelA) and Rta at the C terminus. An effector that comprises VP64, p65 and Rta is referred to as “VPR.” The GenBank Accession number of VP64 is ADD60007.1, the GenBank Accession number of p65 is NP_001138610.1, and the GenBank Accession number of Rta is AAA66528.1.

An exemplary amino acid sequence of a VPR is as follows:

(SEQ ID NO.: 66)
DALDDFDLDMLGSDALDDFDLDMLGSDALDDFDLDMLGSDALDDFDLDML
SGGPKKKRKVGSQYLPDTDDRHRIEEKRKRTYETFKSIMKKSPFSGPTDP
RPPPRRIAVPSRSSASVPKPAPQPYPFTSSLSTINYDEFPTMVFPSGQIS
QASALAPAPPQVLPQAPAPAPAPAMVSALAQAPAPVPVLAPGPPQAVAPP
APKPTQAGEGTLSEALLQLQFDDEDLGALLGNSTDPAVFTDLASVDNSEF
QQLLNQGIPVAPHTTEPMLMEYPEAITRLVTGAQRPPDPAPAPLGAPGLP
NGLLSGDEDFSSIADMDFSALLGSGSGSRDSREGMFLPKPEAGSAISDVF
EGREVCQPKRLRPFHPPGSPWANRPLPASLAPTPTGPVHEPVGSLTPAPV
PQPLDPAPAVTPEASHLLEDPDEETSQAVKALREMADTVIPQKEEAAICG
QMDLSHPPPRGHLDELTTTLESMTEDLNLDSPLTPELNEILDTFLNDECL
LHAMHISTGLSIFDTSLF

As used herein, “p300 core domain” refers to the catalytic core of the human acetyltransferase p300. The GenBank Accession number for the protein comprising p300 is NP_001420.2.

An exemplary amino acid sequence of a p300 is as follows:

(SEQ ID NO.: 67)
IFKPEELRQALMPTLEALYRQDPESLPFRQPVDPQLLGIPDYEDIVKSPM
DLSTIKRKLDTGQYQEPWQYVDDIWLMFNNAWLYNRKTSRVYKYCSKLSE
VFEQEIDPVMQSLGYCCGRKLEFSPQTLCCYGKQLCTIPRDATYYSYQNR
YHFCEKCFNEIQGESVSLGDDPSQPQTTINKEQFSKRKNDTLDPELFVEC
TECGRKMHQICVLHHEIIWPAGFVCDGCLKKSARTRKENKFSAKRLPSTR
LGTFLENRVNDFLRRQNHPESGEVTVRVVHASDKTVEVKPGMKARFVDSG
EMAESFPYRTKALFAFEEIDGVDLCFFGMHVQEYGSDCPPPNQRRVYISY
LDSVHFFRPKCLRTAVYHEILIGYLEYVKKLGYTTGHIWACPPSEGDDYI
FHCHPPDQKIPKPKRLQEWYKKMLDKAVSERIVHDYKDIFKQATEDRLTS
AKELPYFEGDFWPNVLEESIKELEQEEEERKREENTSNESTDVTKGDSKN
AKKKNNKKTSKNKSSLSRGNKKKPGMPNVSNDLSQKLYATMEKHKEVFFV
IRLIAGPAANSLPPIVDPDPLIPCDLMDGRDAFLTLARDKHLEFSSLRRA
QWSTMCMLVELHTQSQD.

As used herein, “KRAB” refers to a Kruppel associated box (KRAB) transcriptional repression domain present in human zinc finger protein-based transcription factors (KRAB zinc finger proteins).

As used herein, MeCp2” refers to methyl CpG binding protein 2 which represses transcription, e.g., by binding to a promoter comprising methylated DNA.

In one embodiment, an epigenetic CpG modifier methylates DNA and inactivates or represses transcription. In some embodiments, a suitable epigenetic CpG modifier for use in the agent, compositions, and methods of the invention comprises a MQ1 domain or a DNMT3a-3L domain.

In one embodiment, an epigenetic CpG modifier demethylates DNA and activates or stimulates transcription. In some embodiments, a suitable epigenetic recruiter for use in the agent, compositions, and methods of the invention comprises a TET1 or TET2 domain.

As used herein “TET1” refers to “ten-eleven translocation methylcytosine dioxygenase 1,” a member of the TET family of enzymes, encoded by the TET1 gene. TET1 is a dioxygenase that catalyzes the conversion of the modified DNA base 5-methylcytosine (5-mC) to 5-hydroxymethylcytosine (5-hmC) by oxidation of 5-mC in an iron and alpha-ketoglutarate dependent manner, the initial step of active DNA demethylation in mammals. Methylation at the C5 position of cytosine bases is an epigenetic modification of the mammalian genome which plays an important role in transcriptional regulation. In addition to its role in DNA demethylation, plays a more general role in chromatin regulation. Preferentially binds to CpG-rich sequences at promoters of both transcriptionally active and Polycomb-repressed genes. Involved in the recruitment of the O-GlcNAc transferase OGT to CpG-rich transcription start sites of active genes, thereby promoting histone H2B GlcNAcylation by OGT.

As used herein, “TET2” refers to “ten-eleven translocation 2 (TET2),” a member of the TET family of enzymes, encoded by the TET1 gene. Similarly to TET1, TET2 is a dioxygenase that catalyzes the conversion of the modified genomic base 5-methylcytosine (5mC) into 5-hydroxymethylcytosine (5hmC) and plays a key role in active DNA demethylation. TET2 a preference for 5-hydroxymethylcytosine in CpG motifs. TET2 also mediates subsequent conversion of 5hmC into 5-formylcytosine (5fC), and conversion of 5fC to 5-carboxylcytosine (5caC). The conversion of 5mC into 5hmC, 5fC and 5caC probably constitutes the first step in cytosine demethylation. Methylation at the C5 position of cytosine bases is an epigenetic modification of the mammalian genome which plays an important role in transcriptional regulation. In addition to its role in DNA demethylation, also involved in the recruitment of the O-GlcNAc transferase OGT to CpG-rich transcription start sites of active genes, thereby promoting histone H2B GlcNAcylation by OGT.

As used herein “DNMT3a-3L” refers to a fusion of a DNA methyltransferase, Dnmt3a and a Dnmt3L which is catalytically inactive, but directly interacts with the catalytic domains of Dnmt3a.

In some embodiments, a suitable epigenetic recruiter for use in the agent, compositions, and methods of the invention comprises a MQ1 domain, a DNMT3a-3L domain, a TET1 domain, or a TET2 domain. In one embodiment, a suitable epigenetic recruiter for use in the agent, compositions, and methods of the invention comprises a dCas9-MQ1 fusion, a dCas9-DNMT3a-3L fusion, a dCas9-TET1 fusion or a dCAS9-TET2 fusion.

### III. DELIVERY OF A SITE-SPECIFIC HNF4α DISRUPTING AGENT OF THE INVENTION AND COMPOSITIONS COMPRISING A SITE-SPECIFIC AN HNF4α DISRUPTING AGENTS OF THE INVENTION

The delivery of the disrupting agents of the invention to a cell e.g., a cell within a subject, such as a human subject (e.g., a subject in need thereof, such as a subject having an HNF4α-associated disorder, e.g., cirrhosis) may be achieved in a number of different ways. For example, delivery may be performed by contacting a cell with a disrupting agent of the invention either in vitro, ex vivo, or in vivo. In vivo delivery may be performed directly by administering a composition, such as a lipid composition, comprising a disrupting agent to a subject. Alternatively, in vivo delivery may be performed indirectly by administering one or more vectors that encode and direct the expression of the disrupting agent. These alternatives are discussed further below.

In some embodiments, the disrupting agent comprises a nucleic acid molecule encoding a fusion protein, the fusion protein comprising a site-specific HNF4α targeting moiety, such as a polynucleotide encoding a DNA-binding domain of a Transcription activator-like effector (TALE) polypeptide or a zinc finger (ZNF) polypeptide, or fragment thereof, that specifically targets and binds to the HNF4α expression control region and an effector molecule, such as a VPR.

In other embodiments, the disrupting agent comprises a guide RNA and an mRNA encoding an effector molecule. The ratio of guide RNA to mRNA may be about 100:1 to about 1:100 (wt:wt).

In general, any method of delivery of a site-specific HNF4α disrupting agent of the invention (in vitro, ex vivo, or in vivo) may be adapted for use with the disrupting agents of the invention (see e.g., Akhtar S. and Julian R L., (1992) Trends Cell. Biol. 2(5):139-144 and WO94/02595, which are incorporated herein by reference in their entireties). For in vivo delivery, factors to be considered for delivering a site-specific HNF4α disrupting agent of the invention include, for example, biological stability of the disrupting agent, prevention of non-specific effects, and accumulation of the disrupting agent in the target tissue. The non-specific effects of a disrupting agent can be minimized by local administration, for example, by direct injection or implantation into a tissue or topically administering a composition comprising the disrupting agent. Local administration to a treatment site maximizes local concentration of the disrupting agent, limits the exposure of the disrupting agent to systemic tissues that can otherwise be harmed by the disrupting agent or that can degrade the disrupting agent, and permits a lower total dose of the disrupting agent to be administered.

For administering a site-specific HNF4α disrupting agent systemically for the treatment of a disease, such as an HNF4α-associate disease, the disrupting agent, e.g., a disrupting agent comprising a site-specific targeting moiety comprising a nucleic acid molecule, can be modified or alternatively delivered using a drug delivery system; both methods act to prevent the rapid degradation of a site-specific targeting moiety comprising a nucleic acid molecule by endo- and exo-nucleases in vivo. Modification of a disrupting agent comprising a site-specific targeting moiety comprising a nucleic acid molecule or a pharmaceutical carrier also permits targeting of the disrupting agent to a target tissue and avoidance of undesirable off-target effects. For example, a disrupting agent of the invention may be modified by chemical conjugation to lipophilic groups such as cholesterol to enhance cellular uptake and prevent degradation.

Alternatively, a disrupting agent of the invention may be delivered using a drug delivery system such as a nanoparticle, a dendrimer, a polymer, a liposome, or a cationic delivery system. Positively charged cationic delivery systems facilitate binding of disrupting agent (e.g., negatively charged molecule) and also enhance interactions at the negatively charged cell membrane to permit efficient uptake of a disrupting agent by the cell. Cationic lipids, dendrimers, or polymers can either be bound to a disrupting agent, or induced to form a vesicle or micelle (see e.g., Kim S H. et al., (2008) Journal of Controlled Release 129(2):107-116) that encases the disrupting agent. The formation of vesicles or micelles further prevents degradation of the disrupting agent when administered systemically. Methods for making and administering cationic complexes are well within the abilities of one skilled in the art (see e.g., Sorensen, D R., et al. (2003) J. Mol. Biol 327:761-766; Verma, U N. et al., (2003) Clin. Cancer Res. 9:1291-1300; Arnold, A S et al. (2007) J. Hypertens. 25:197-205, which are incorporated herein by reference in their entirety). Some non-limiting examples of drug delivery systems useful for systemic delivery of a disrupting agent of the invention include DOTAP (Sorensen, D R., et al (2003), supra; Verma, U N. et al., (2003), supra), Oligofectamine, “solid nucleic acid lipid particles” (Zimmermann, T S. et al., (2006) Nature 441:111-114), cardiolipin (Chien, P Y. et al., (2005) Cancer Gene Ther. 12:321-328; Pal, A. et al., (2005) Int J. Oncol. 26:1087-1091), polyethyleneimine (Bonnet M E. et al., (2008) Pharm. Res. August 16 Epub ahead of print; Aigner, A. (2006) J. Biomed. Biotechnol. 71659), Arg-Gly-Asp (RGD) peptides (Liu, S. (2006) Mol. Pharm. 3:472-487), and polyamidoamines (Tomalia, D A. et al., (2007) Biochem. Soc. Trans. 35:61-67; Yoo, H. et al., (1999) Pharm. Res. 16:1799-1804). In some embodiments, a disrupting agent (e.g., gRNA, or mRNA) forms a complex with cyclodextrin for systemic administration. Methods for administration and pharmaceutical compositions comprising cyclodextrins may be found in U.S. Pat. No. 7,427,605, the entire contents of which are incorporated herein by reference.

The disrupting agents of the invention may be incorporated into pharmaceutical compositions suitable for administration. Such compositions typically include one or more species of disrupting agent and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The pharmaceutical compositions of the present invention may be administered in a number of ways depending upon whether local or systemic treatment is desired and upon the area to be treated. Administration may be topical (including ophthalmic, vaginal, rectal, intranasal, transdermal), oral, or parenteral. Parenteral administration includes intravenous drip, subcutaneous, intraperitoneal or intramuscular injection, or intrathecal or intraventricular administration.

The route and site of administration may be chosen to enhance delivery or targeting of the disrupting agent comprising a site-specific targeting moiety to a particular location. For example, to target liver cells, intravenous injection may be used. Lung cells may be targeted by administering the disrupting agent in aerosol form. Jejunum cells may be targeted by anal administration.

Formulations for topical administration may include transdermal patches, ointments, lotions, creams, gels, drops, suppositories, sprays, liquids, and powders. Conventional pharmaceutical carriers, aqueous, powder or oily bases, thickeners and the like may be necessary or desirable. Coated condoms, gloves and the like may also be useful.

Compositions for oral administration include powders or granules, suspensions or solutions in water, syrups, elixirs or non-aqueous media, tablets, capsules, lozenges, or troches. In the case of tablets, carriers that can be used include lactose, sodium citrate and salts of phosphoric acid. Various disintegrants such as starch, and lubricating agents such as magnesium stearate, sodium lauryl sulfate and talc, are commonly used in tablets. For oral administration in capsule form, useful diluents are lactose and high molecular weight polyethylene glycols. When aqueous suspensions are required for oral use, the nucleic acid compositions can be combined with emulsifying and suspending agents. If desired, certain sweetening or flavoring agents can be added.

Compositions for intravenous administration may include sterile aqueous solutions which may also contain buffers, diluents, and other suitable additives.

Formulations for parenteral administration may include sterile aqueous solutions which may also contain buffers, diluents, and other suitable additives. For intravenous use, the total concentration of solutes may be controlled to render the preparation isotonic.

In one embodiment, the administration of a disrupting agent composition of the invention is parenteral, e.g., intravenous (e.g., as a bolus or as a diffusible infusion), intradermal, intraperitoneal, intramuscular, intrathecal, intraventricular, intracranial, subcutaneous, transmucosal, buccal, sublingual, endoscopic, rectal, oral, vaginal, topical, pulmonary, intranasal, urethral, or ocular. Administration can be provided by the subject or by another person, e.g., a health care provider. The composition may be provided in measured doses or in a dispenser which delivers a metered dose. Selected modes of delivery are discussed in more detail below.

In certain embodiments, the disrupting agents of the invention are polynucleotides, such as mRNAs, and are formulated in lipid nanoparticles (LNPs).

### A. Compositions Comprising a Site-Specific an HNF4α Disrupting Agent of the Invention

The site-specific HNF4α disrupting agents of the invention may be formulated into compositions, such as pharmaceutical compositions, using one or more excipients to: (1) increase stability; (2) increase cell transfection; (3) permit sustained or delayed release (e.g., from a depot formulation); (4) alter the biodistribution (e.g., target the disrupting agent to specific tissues or cell types); (5) increase the translation of an encoded protein in vivo; and/or (6) alter the release profile of an encoded protein in vivo. In addition to traditional excipients, such as any and all solvents, dispersion media, diluents, or other liquid vehicles, dispersion or suspension aids, surface active agents, isotonic agents, thickening or emulsifying agents, preservatives, excipients for use in the compositions of the invention may include, without limitation, lipidoids, liposomes, lipid nanoparticles, polymers, lipoplexes, core-shell nanoparticles, peptides, proteins, cells transfected with nucleic acid molecules, modified nucleic acid molecules, or RNA (e.g., for transplantation into a subject), hyaluronidase, nanoparticle mimics and combinations thereof. Accordingly, the pharmaceutical compositions of the invention can include one or more excipients, each in an amount that together increases the stability of the disrupting agent, increases cell transfection by the disrupting agent, increases the expression of modified nucleic acid, or mRNA encoded protein, and/or alters the release profile of a disrupting agent. Further, the disrupting agents of the present invention may be formulated using self-assembled nucleic acid nanoparticles (see, e.g., U.S. Patent Publication No. 2016/0038612A1, which is incorporated herein by reference in its entirety).

i. Lipidoid

The synthesis of lipidoids has been extensively described and formulations containing these compounds are particularly suited for delivery of a disrupting agent of the invention, such as a disrupting agent comprising a site-specific HNF4α targeting moiety comprising a nucleic acid molecule, e.g., comprising modified nucleic acid molecules or mRNA (see Mahon et al., Bioconjug Chem. 2010 21:1448-1454; Schroeder et al., J Intern Med. 2010 267:9-21; Akinc et al., Nat Biotechnol. 2008 26:561-569; Love et al., Proc Natl Acad Sci USA. 201 0 107: 1864-1869; Siegwart et al., Proc Natl Acad Sci USA. 2011108:12996-3001; the contents of all of which are incorporated herein in their entireties).

For example, lipidoids have been used to effectively deliver double stranded small interfering RNA molecules, single stranded nucleic acid molecules, modified nucleic acid molecules or modified mRNA. (See, e.g., US Patent Publication 2016/0038612A1). Complexes, micelles, liposomes or particles can be prepared containing these lipidoids and, therefore, provide effective delivery of a site-specific HNF4α targeting moiety comprising a nucleic acid molecule, as judged by the production of an encoded protein, following the administration of a lipidoid formulation, e.g., via localized and/or systemic administration. Lipidoid complexes of can be administered by various means including, but not limited to, intravenous, intramuscular, intradermal, intraperitoneal or subcutaneous routes.

In vivo delivery of a site-specific HNF4α targeting moiety comprising, e.g., a nucleic acid molecule, may be affected by many parameters, including, but not limited to, the formulation composition, nature of particle PEGylation, degree of loading, polynucleotide to lipid ratio, and biophysical parameters such as, but not limited to, particle size (Akinc et al., Mol Ther. 2009 17:872-879; herein incorporated by reference in its entirety). As an example, small changes in the anchor chain length of poly(ethylene glycol) (PEG) lipids may result in significant effects on in vivo efficacy. Formulations with different lipidoids, including, but not limited to penta[3-(1-laurylaminopropiony I)]-triethylenetetramine hydrochloride (TETA-5LAP; aka 98NI2-5, see Murugaiah et al., Analytical Biochemistry, 401:61 (2010); the contents of which are herein incorporated by reference in their entirety), C12-200 (including derivatives and variants), and MD1, may be used.

In one embodiment, a disrupting agent comprising a site-specific HNF4α targeting moiety comprising, e.g., a nucleic acid molecule, is formulated with a lipidoid for systemic intravenous administration to target cells of the liver. For example, a final optimized intravenous formulation comprising a disrupting agent comprising a site-specific HNF4α targeting moiety comprising a nucleic acid molecule, and a lipid molar composition of 42% 98NI2-5, 48% cholesterol, and 10% PEG-lipid with a final weight ratio of about 7.5 to 1 total lipid to nucleic acid molecule, and a C14 alkyl chain length on the PEG lipid, with a mean particle size of roughly 50-60 nm, can result in the distribution of the formulation to be greater than 90% to the liver (see, Akinc et al., Mol Ther. 2009 17:872-879; the contents of which is herein incorporated by reference in its entirety). In another example, an intravenous formulation using a C12-200 lipidoid (see, e.g., PCT Publication No. WO 2010/129709, which is herein incorporated by reference in its entirety) having a molar ratio of 50/10/38.5/1.5 of C12-200/disteroylphosphatidyl choline/cholesterol/PEG-DMG, with a weight ratio of 7 to 1 total lipid to nucleic acid molecule, and a mean particle size of 80 nm may be used to deliver a disrupting agent comprising a site-specific HNF4α targeting moiety comprising a nucleic acid molecule, to hepatocytes (see, Love et al., Proc Natl Acad Sci USA. 2010 107:1864-1869; the contents of which are herein incorporated by reference in their entirety). In another embodiment, an MD1 lipidoid-containing formulation may be used to effectively deliver a disrupting agent comprising a site-specific HNF4α targeting moiety comprising a nucleic acid molecule, to hepatocytes in vivo. The characteristics of optimized lipidoid formulations for intramuscular or subcutaneous routes may vary significantly depending on the target cell type and the ability of formulations to diffuse through the extracellular matrix into the blood stream. While a particle size of less than 150 nm may be desired for effective hepatocyte delivery due to the size of the endothelial fenestrae (see, Akinc et al., Mol Ther. 2009 17:872-879; the contents of which are herein incorporated by reference in their entirety), use of lipidoid-formulated nucleic acid molecules to deliver the formulation to other cells types including, but not limited to, endothelial cells, myeloid cells, and muscle cells may not be similarly size-limited. Use of lipidoid formulations to deliver siRNA in vivo to other non-hepatocyte cells such as myeloid cells and endothelium has been reported (see Akinc et al., Nat Biotechnol. 200826:561-569; Leuschner et al., Nat Biotechnol. 2011 29: 1005-101 0; Cho et al. Adv. Funct. Mater. 2009 19:3112-3118; 8th International Judah Folkman Conference, Cambridge, Mass. Oct. 8-9, 2010; the contents of each of which are herein incorporated by reference in their entirety). For delivery to myeloid cells, such as monocytes, lipidoid formulations may have a similar component molar ratio. Different ratios of lipidoids and other components including, but not limited to, disteroylphosphatidyl choline, cholesterol and PEG-DMG, may be used to optimize the formulation for delivery to different cell types including, but not limited to, hepatocytes, myeloid cells, muscle cells, etc. For example, the component molar ratio may include, but is not limited to, 50% CI2-200, 10% disteroylphosphatidyl choline, 38.5% cholesterol, and 1.5% PEG-DMG (see Leuschner et al., Nat Biotechnol 2011 29: 1005-101 0; the contents of which are herein incorporated by reference in its entirety). The use of lipidoid formulations for the localized delivery to cells (such as, but not limited to, adipose cells and muscle cells) via either subcutaneous, intradermal or intramuscular delivery, may not require all of the formulation components desired for systemic delivery and, as such, may comprise only the lipidoid and a disrupting agent comprising a site-specific HNF4α targeting moiety comprising, e.g., a nucleic acid molecule, as described herein.

Combinations of different lipidoids may be used to improve the efficacy of the formulations by increasing cell transfection and/or increasing the translation of encoded protein contained therein (see Whitehead et al., Mol. Ther. 2011, 19:1688-1694, the contents of which are herein incorporated by reference in their entirety).

In one embodiment, the lipidoid may be prepared from the conjugate addition of alkylamines to acrylates. As a non-limiting example, a lipidoid may be prepared by the methods described in PCT Patent Publication No. WO 2014/028487, the contents of which are herein incorporated by reference in its entirety. In one embodiment, the lipidoid may comprise a compound having formula (I), formula (II), formula (III), formula (IV) or formula (V) as described in PCT Patent Publication No. WO 2014/028487, the contents of which are herein incorporated by reference in their entirety. In one embodiment, the lipidoid may be biodegradable.

ii. Liposomes, Lipoplexes, and Lipid Nanoparticles

A disrupting agent of the invention may be formulated using one or more liposomes, lipoplexes, or lipid nanoparticles. In one embodiment, pharmaceutical compositions of the invention include liposomes. Liposomes are artificially-prepared vesicles which are primarily composed of a lipid bilayer and may be used as a delivery vehicle for the administration of nutrients and pharmaceutical formulations. Liposomes may be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter. Liposome design may include, but is not limited to, opsonins or ligands in order to improve the attachment of liposomes to unhealthy tissue or to activate events such as, but not limited to, endocytosis. Liposomes may contain a low or a high pH in order to improve the delivery of the pharmaceutical formulations. The formation of liposomes may depend on the physicochemical characteristics such as, but not limited to, the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products.

As a non-limiting example, liposomes, such as synthetic membrane vesicles, may be prepared by the methods, apparatus and devices described in U.S. Patent Publication Nos. 2013/0177638, 2013/0177637, 2013/0177636, 201/30177635, 2013/0177634, 2013/0177633, 2013/0183375, 2013/0183373, 2013/0183372 and 2016/0038612) and PCT Patent Publication No WO 2008/042973, the contents of each of which are herein incorporated by reference in their entirety.

In one embodiment, a pharmaceutical composition described herein may include, without limitation, liposomes such as those formed from 1,2-dioleyloxy-N,N-dimethyl ami-nopropane (DODMA) liposomes, DiLa2 liposomes from Marina Biotech (Bothell, Wash.), 1,2-dilinoleyloxy-3-dimethylaminopropane (DLin-DMA), 2,2-dilinoleyl-4-(2-dimethylaminoethyl)-[1,3]-dioxolane (DLin-KC2-DMA), and MC3 (US20100324120; herein incorporated by reference in its entirety) and liposomes which may deliver small molecule drugs such as, but not limited to, DOXIL® from Janssen Biotech, Inc. (Horsham, Pa.). In one embodiment, a pharmaceutical composition described herein may include, without limitation, liposomes such as those formed from the synthesis of stabilized plasmid-lipid particles (SPLP) or stabilized nucleic acid lipid particle (SNALP) that have been previously described and shown to be suitable for oligonucleotide delivery in vitro and in vivo (see Wheeler et al. Gene Therapy. 1999 6:271-281; Zhang et al. Gene Therapy. 19996:1438-1447; Jeffs et al. Pharm Res. 2005 22:362-372; Morrissey et al., Nat Biotechnol. 2005 2:1002-1007; Zimmermann et al., Nature. 2006 441:111-114; Heyes et al. J Contr Rel. 2005 107:276-287; Semple et al. Nature Biotech. 2010 28:172-176; Judge et al. J Clin Invest. 2009 119:661-673; deFougerolles Hum Gene Ther. 2008 19:125-132; U.S. Patent Publication Nos 2013/0122104, 2013/0303587, and 2016/0038612; the contents of each of which are incorporated herein in their entireties). The original manufacturing method of Wheeler et al. was a detergent dialysis method, which was later improved by Jeffs et al. and is referred to as the spontaneous vesicle formation method. The liposome formulations of the invention may be composed of 3 to 4 lipid components in addition a disrupting agent comprising a site-specific HNF4α targeting moiety. As an example a liposome of the invention can contain, but is not limited to, 55% cholesterol, 20% disteroylphosphatidyl choline (DSPC), 10% PEG-SDSG, and 15% 1,2-dioleyloxy-N,N-dimethylaminopropane (DODMA), as described by Jeffs et al. As another example, liposome formulations of the invention may contain, but are not limited to, 48% cholesterol, 20% DSPC, 2% PEG-c-DMA, and 30% cationic lipid, where the cationic lipid can be 1,2-distearloxy-N,N-dimethylaminopropane (DSDMA), DODMA, DLin-DMA, or 1,2-dilinolenyloxy-3-dimethylaminopropane (DLenDMA), as described by Heyes et al. In some embodiments, liposome formulations may comprise from about 25.0% cholesterol to about 40.0% cholesterol, from about 30.0% cholesterol to about 45.0% cholesterol, from about 35.0% cholesterol to about 50.0% cholesterol and/or from about 48.5% cholesterol to about 60% cholesterol. In another embodiment, formulations of the invention may comprise a percentage of cholesterol selected from the group consisting of 28.5%, 31.5%, 33.5%, 36.5%, 37.0%, 38.5%, 39.0% and 43.5%. In some embodiments, liposome formulations of the invention may comprise from about 5.0% to about 10.0% DSPC and/or from about 7.0% to about 15.0% DSPC.

In one embodiment, a pharmaceutical composition may include liposomes which may be formed to deliver a disrupting agent of the invention. The disrupting agent comprising a site-specific HNF4α targeting moiety comprising may be encapsulated by the liposome and/or it may be contained in an aqueous core which may then be encapsulated by the liposome (see, e.g., PCT Patent Publication Nos. WO 2012/031046, WO 2012/031043, WO 2012/030901 and WO 2012/006378 and U.S. Patent Publication Nos. 2013/0189351, 2013/0195969 and 201/30202684, the contents of each of which are herein incorporated by reference in their entirety).

In another embodiment, liposomes for use in the present invention may be formulated for targeted delivery. As a non-limiting example, the liposome may be formulated for targeted delivery to the liver. Such a liposome may include, but is not limited to, a liposome described in U.S. Patent Publication No. 2013/0195967, the contents of which are herein incorporated by reference in their entirety.

In one embodiment, formulations comprising liposomes and a disrupting agent may be administered intramuscularly, intradermally, or intravenously.

In another embodiment, a lipid formulation of the invention may include at least one cationic lipid, a lipid which enhances transfection and a least one lipid which contains a hydrophilic head group linked to a lipid moiety (International Pub. No. WO2011076807 and U.S. Pub. No. 20110200582; the contents of each of which is herein incorporated by reference in their entirety). In another embodiment, a lipid formulation of the invention is a lipid vesicle which may have crosslinks between functionalized lipid bilayers (see U.S. Patent Publication No. 2012/0177724, the contents of which are herein incorporated by reference in their entirety).

In one embodiment, a formulation comprising a disrupting agent is a lipid nanoparticle (LNP) which may comprise at least one lipid. The lipid may be selected from, but is not limited to, DLin-DMA, DLin-K-DMA, 98NI2-5, C12-200, DLin-MC3-DMA, DLin-KC2-DMA, DODMA, PLGA, PEG, PEG-DMG, PEGylated lipids and amino alcohol lipids. In another aspect, the lipid may be a cationic lipid such as, but not limited to, DLin-DMA, DLin-D-DMA, DLin-MC3-DMA, DLin-KC2-DMA, DODMA and amino alcohol lipids. The amino alcohol cationic lipid may be the lipids described in and/or made by the methods described in U.S. Patent Publication No. 2013/0150625.

In one embodiment, the cationic lipid may be selected from, but not limited to, a cationic lipid described in PCT Publication Nos. WO 2012/040184, WO 2011/153120, WO 2011/149733, WO 2011/090965, WO 2011/043913, WO 2011/022460, WO 2012/061259, WO 2012/054365, WO 2012/044638, WO 2010/080724, WO 2010/21865, WO 2008/103276, WO 2013/086373 and WO 2013/086354, U.S. Pat. Nos. 7,893,302, 7,404,969, 8,283, 333, 8,466,122 and 8,569,256, and U.S. Patent Publication Nos. 2010/0036115, 2012/0202871, 2013/0064894, 2013/0129785, 2013/0150625, 2013/0178541, 2013/0225836 and 2014/0039032; the contents of each of which are herein incorporated by reference in their entirety. In another embodiment, the cationic lipid may be selected from, but not limited to, formula A described in PCT Publication Nos. WO 2012/040184, WO 0111/53120, WO 2011/149733, WO 2011/090965, WO 2011/043913, WO 2011/022460, WO 2012/061259, WO 2012/054365, WO 2012/044638 and WO 2013/116126 or U.S. Patent Publication Nos. 2013/0178541 and 2013/0225836; the contents of each of which is herein incorporated by reference in their entirety. In yet another embodiment, the cationic lipid may be selected from, but not limited to, formula CLI-CLXXIX of PCT Publication No. WO 2008/103276, formula CLICLXXIX of U.S. Pat. No. 7,893,302, formula CLICLXXXXII of U.S. Pat. No. 7,404,969 and formula I-VI of us Patent Publication No. 2010/0036115, formula I of U.S. Patent Publication No 2013/0123338; each of which is herein incorporated by reference in their entirety.

In one embodiment, the cationic lipid may be synthesized by methods known in the art and/or as described in PCT Publication Nos. WO 2012/040184 WO 2011/153120, WO 2011/149733, WO 2011/090965: WO 2011/043913, WO 2011/022460, WO 2012/061259, WO 2012/054365, WO 2012/044638, WO 2010/080724, WO 2010/21865, WO 2013/126803, WO 2013/086373, and WO 2013/086354; the contents of each of which are herein incorporated by reference in their entirety.

In one embodiment, the lipids which may be used in the formulations and/or for delivery of the disrupting agents described herein may be a cleavable lipid. As a non-limiting example, a cleavable lipid and/or pharmaceutical compositions comprising cleavable lipids include those described in PCT Patent Publication No. WO 2012/170889, the contents of which are herein incorporated by reference in their entirety. As another non-limiting example, the cleavable lipid may be HGT4001, HGT4002, HGT4003, HGT4004 and/or HGT4005 as described in PCT Patent Publication No. WO 2012/170889, the contents of which are herein incorporated by reference in their entirety.

In one embodiment, polymers which may be used in the formulation and/or delivery of the disrupting agents described herein may include, but is not limited to, poly(ethylene) glycol (PEG), polyethylenimine (PEI), dithiobis(succinimidylpropionate) (DSP), Dimethy 1-3,3′-dithiobispropionimidate (DTBP), poly(ethylene imine) biscarbamate (PEIC), poly(L-lysine) (PLL), histidine modified PLL, poly(N-vinylpyrrohdone) (PVP), poly(propylenimine (PPI), poly(amidoamine) (PAMAM), poly(amido ethylenimine) (SS-PAEI), triehtylenetetramine (TETA), poly(O-aminoester), poly(4-hydroxy-L-proine ester) (PHP), poly(allylamine), poly(α-[4-aminobutyl]-L-glycolic acid (PAGA), Poly(D,L-lactic-coglycolid acid (PLGA), Poly(N-ethyl-4-vinylpyridinium bromide), poly(phosphazene)s (PPZ), poly(phosphoester)s (PPE), poly(phosphoramidate)s (PPA), poly(N-2-hydroxypropylmethacrylamide) (pHPMA), poly(2-(dimethylamino)ethyl methacrylate) (pDMAEMA), poly(2-aminoethyl propylene phosphate) PPE_EA), Chitosan, galactosylated chitosan, N-dodecylated chitosan, histone, collagen and dextran-spermine. In one embodiment, the polymer may be an inert polymer such as, but not limited to, PEG. In one embodiment, the polymer may be a cationic polymer such as, but not limited to, PEl, PLL, TETA, poly(allylamine), Poly(N-ethyl-4-vinylpyridinium bromide), pHPMA and pDMAEMA. In one embodiment, the polymer may be a biodegradable PEl such as, but not limited to, DSP, DTBP and PEIC. In one embodiment, the polymer may be biodegradable such as, but not limited to, histine modified PLL SSPAEI, poly(O-aminoester), PHP, PAGA, PLGA, PPZ, PPE, PPA and PPE-EA.

In one embodiment, an LNP formulation of the invention may be prepared according to the methods described in PCT Publication Nos. WO 2011/127255 or WO 2008/103276, the contents of each of which are herein incorporated by reference in their entirety. As a non-limiting example, a disrupting agent comprising a site-specific HNF4α targeting moiety may be encapsulated in an LNP formulation as described in PCT Publication Nos. WO 2011/127255 and/or WO 2008/103276; the contents of each of which are herein incorporated by reference in their entirety. As another non-limiting example, a disrupting agent comprising a site-specific HNF4α targeting moiety as described herein, may be formulated in a nanoparticle to be delivered by a parenteral route as described in U.S. Patent Publication No. 2012/0207845 and PCT Publication No. WO 2014/008334; the contents of each of which are herein incorporated by reference in their entirety.

In one embodiment, LNP formulations described herein may be administered intramusculary. The LNP formulation may comprise a cationic lipid described herein, such as, but not limited to, DLin-DMA, DLin-KC2-DMA, DLin-MC3-DMA, DODMA and C12-200.

In one embodiment, LNP formulations described herein comprising a disrupting agent as described herein, may be administered intradermally. The LNP formulation may comprise a cationic lipid described herein, such as, but not limited to, DLin-DMA, DLin-KC2-DMA, DLin-MC3-DMA, DODMA and C12-200.

The nanoparticle formulations may comprise conjugate, such as a phosphate conjugate, a polymer conjugates, a conjugate that enhances the delivery of nanoparticle as described in US Patent Publication No. US20160038612 A1.

In one embodiment, the lipid nanoparticle formulation comprises DLin-MC3-DMA as described in US Patent Publication No. US20100324120.

In one embodiment, the lipid nanoparticle comprises a lipid compound, or a pharmaceutically acceptable salt, tautomer or stereoisomer thereof, or a lipid nanoparticle formulation, as described in U.S. Pat. No. 10,723,692B2, US Patent Publication Nos. US20200172472A1, US20200163878A1, US20200046838A1, US20190359556A1, US20190314524A1, US20190274968A1, US20190022247A1, US20180303925A1, US20180185516A1, US20160317676A1, International Patent Publication No.: WO20200146805A1, WO2020081938A1, WO2019089828A1, WO2019036030A1, WO2019036028A1, WO2019036008A1, WO 2018200943A1, WO2018191719A1, WO2018107026A1, WO2018081480A1, the contents of each of which are herein incorporated by reference in their entirety (Acuitas Therapeutics, Inc.).

In one embodiment, the lipid nanoparticle comprises an amino lipid, or a pharmaceutically acceptable salt, tautomer or stereoisomer thereof, or a lipid nanoparticle formulation, described by Tekmira Pharmaceuticals Corp. in U.S. Pat. No. 9,139,554B2, U.S. Pat. No. 9,051,567B2, U.S. Pat. No. 8,883,203B2, US Patent Publication US20110117125A1, the contents of each of which are herein incorporated by reference in their entirety. In one particular example, the compound described in U.S. Pat. No. 9,139,554B2 is DLin-kC2-DMA.

In one embodiment, the lipid nanoparticle comprises an amino lipid, or a pharmaceutically acceptable salt, tautomer or stereoisomer thereof, or a lipid nanoparticle formulation, described by Arbutus Biopharma Corp. in U.S. Ser. No. 10/561,732B2, U.S. Pat. No. 9,938,236B2, U.S. Pat. No. 9,687,550B2, US Patent Publication US20190240354A1, US20170027658A1, WO2020097493A1, WO2020097520A1, WO2020097540A1, WO2020097548A1, the contents of each of which are herein incorporated by reference in their entirety.

Lipid nanoparticles may be engineered to alter the surface properties of particles so the lipid nanoparticles may penetrate the mucosal barrier. Mucus is located on mucosal tissue such as, but not limited to, oral (e.g., the buccal and esophageal membranes and tonsil tissue), ophthalmic, gastrointestinal (e.g., stomach, small intestine, large intestine, colon, rectum), nasal, respiratory (e.g., nasal, pharyngeal, tracheal and bronchial membranes), genital (e.g., vaginal, cervical and urethral membranes). Nanoparticles larger than 10-200 nm which are preferred for higher drug encapsulation efficiency and the ability to provide the sustained delivery of a wide array of drugs have been thought to be too large to rapidly diffuse through mucosal barriers. Mucus is continuously secreted, shed, discarded or digested and recycled so most of the trapped particles may be removed from the mucosla tissue within seconds or within a few hours. Large polymeric nanoparticles (200 nm-500 nm in diameter) which have been coated densely with a low molecular weight polyethylene glycol (PEG) diffused through mucus only 4 to 6-fold lower than the same particles diffusing in water (Lai et al. PNAS 2007 104(5): 1482-487; Lai et al. Adv Drug Deliv Rev. 200961(2): 158-171; the contents of each of which are herein incorporated by reference in their entirety). The transport of nanoparticles may be determined using rates of permeation and/or fluorescent microscopy techniques including, but not limited to, fluorescence recovery after photobleaching (FRAP) and high resolution multiple particle tracking (MPT). As a non-limiting example, compositions which can penetrate a mucosal barrier may be made as described in U.S. Pat. No. 8,241,670 or International Patent Publication No. WO2013110028, the contents of each of which are herein incorporated by reference in their entirety.

In one embodiment, a disrupting agent comprising a site-specific HNF4α targeting moiety as described herein, is formulated as a lipoplex, such as, without limitation, the ATUPLEX™ system, the DACC system, the DBTC system and other siRNAlipoplex technology from Silence Therapeutics (London, United Kingdom), STEMFECFM from STEMGENT® (Cambridge, Mass.), and polyethylenimine (PEl) or protamine-based targeted and non-targeted delivery of nucleic acids acids (Aleku et al. Cancer Res. 2008 68:9788-9798; Strumberg et al. Int J Clin Pharmacol Ther 2012 50:76-78; Santel et al., Gene Ther 2006 13:1222-1234; Santel et al., Gene Ther 200613:1360-1370; Gutbier et al., PulmPharmacol. Ther. 201023:334-344; Kaufmann et al. Microvasc Res 2010 80:286-293; Weide et al. J Immunother. 2009 32:498-507; Weide et al. J Immnnother. 2008 31:180-188; Pascolo Expert Opin. Biol. Ther. 4:1285-1294; Fotin-Mleczek et al., 2011 J. Immunother. 34: 1-15; Song et al., Nature Biotechnol. 2005,23:709-717; Peer et al., Proc Natl Acad Sci USA. 2007 6; 104:4095-4100; deFougerolles Hum Gene Ther. 2008 19: 125-132; all of which are incorporated herein by reference in their entirety).

In one embodiment such formulations may also be constructed or compositions altered such that they passively or actively are directed to different cell types in vivo, including but not limited to hepatocytes, immune cells, tumor cells, endothelial cells, antigen presenting cells, and leukocytes (Akinc et al. Mol Ther. 2010 18:1357-1364; Song et al., Nat Biotechnol. 2005 23:709-717; Judge et al., J Clin Invest. 2009 119:661-673; Kaufmann et al., Microvasc Res 2010 80:286-293; Santel et al., Gene Ther 200613:1222-1234; Santel et al., Gene Ther 2006 13: 1360-1370; Gutbier et al., Pulm Pharmacol. Ther. 2010 23:334-344; Basha et al., Mol. Ther. 2011 19:2186-2200; Fenske and Cullis, Expert Opin Drug Deliv. 20085:25-44; Peer et al., Science. 2008 319:627-630; Peer and Lieberman, Gene Ther. 2011 18: 1127-1133; all of which are incorporated herein by reference in its entirety). One example of passive targeting of formulations to liver cells includes the DLin-DMA, DLin-KC2-DMA and DLin-MC3-DMA-based lipid nanoparticle formulations which have been shown to bind to apolipoprotein E and promote binding and uptake of these formulations into hepatocytes in vivo (Akinc et al. Mol Ther. 2010 18: 1357-1364; the contents of which are herein incorporated by reference in its entirety). Formulations can also be selectively targeted through expression of different ligands on their surface as exemplified by, but not limited by, folate, transferrin, N-acetylgalactosamine (GalNAc), and antibody targeted approaches (Kolhatkar et al., Curr Drug Discov Technol. 2011 8: 197-206; Musacchio and Torchilin, Front Biosci. 201116: 1388-1412; Yu et al., Mol Membr Biol. 2010 27:286-298; Patil et al., Crit Rev Ther Drug Carrier Syst. 2008 25: 1-61; Benoit et al., Biomacromolecules. 2011 12:2708-2714; Zhao et al., Expert Opin Drug Deliv. 2008 5:309-319; Akinc et al., Mol Ther. 2010 18:1357-1364; Srinivasan et al., Methods Mol Biol. 2012 820: 105-116; Ben-Arie et al., Methods Mol Biol. 2012 757:497-507; Peer 2010 J Control Release. 20:63-68; Peer et al., Proc Natl Acad Sci USA. 2007 104:4095-4100; Kim et al., Methods Mol Biol. 2011 721:339-353; Subramanya et al., Mol Ther. 2010 18:2028-2037; Song et al., Nat Biotechnol. 2005 23:709-717; Peer et al., Science. 2008 319:627-630; Peer and Lieberman, Gene Ther. 2011 18:1127-1133; the contents of all of which are incorporated herein by reference in its entirety).

In one embodiment, a disrupting agent comprising a site-specific HNF4α targeting moiety of the invention, may be formulated as a solid lipid nanoparticle. A solid lipid nanoparticle (SLN) may be spherical with an average diameter between 10 to 1000 nm. SLN possess a solid lipid core matrix that can solubilize lipophilic molecules and may be stabilized with surfactants and/or emulsifiers. In a further embodiment, the lipid nanoparticle may be a self-assembly lipid-polymer nanoparticle (see Zhang et al., ACS Nano, 2008, 2 (8), pp 1696-1702; herein incorporated by reference in its entirety). As a non-limiting example, the SLN may be the SLN described in PCT Publication No. WO2013/105101, the contents of which are herein incorporated by reference in their entirety. As another non-limiting example, the SLN may be made by the methods or processes described in PCT Publication No. WO 2013/105101, the contents of which are herein incorporated by reference in their entirety.

Liposomes, lipoplexes, or lipid nanoparticles may be used to improve the efficacy of a disrupting agent comprising a site-specific HNF4α targeting moiety comprising, e.g., a nucleic acid molecule, to direct protein production as these formulations may be able to increase cell transfection by a nucleic acid molecule; and/or increase the translation of encoded protein (e.g., an effector of the invention). One such example involves the use of lipid encapsulation to enable the effective systemic delivery of polyplex plasmid DNA (Heyes et al., Mol Ther. 2007 15:713-720; the contents of which are herein incorporated by reference in its entirety). The liposomes, lipoplexes, or lipid nanoparticles of the invention may also increase the stability of a disrupting agent comprising a site-specific HNF4α targeting moiety comprising, e.g., a nucleic acid molecule. Liposomes, lipoplexes, or lipid nanoparticles are described in U.S. Patent Publication No. 2016/0038612, the contents of which are incorporated herein by reference in their entirety.

In one embodiment, a disrupting agent comprising a site-specific HNF4α targeting moiety comprising may be formulated for controlled release and/or targeted delivery. As used herein, “controlled release” refers to a pharmaceutical composition or compound release profile that conforms to a particular pattern of release to effect a therapeutic outcome. In one embodiment, a disrupting agent comprising a site-specific HNF4α targeting moiety, as described herein, may be encapsulated into a delivery agent described herein and/or known in the art for controlled release and/or targeted delivery. As used herein, the term “encapsulate” means to enclose, surround or encase. As it relates to the formulation of the compounds of the invention, encapsulation may be substantial, complete or partial. The term “substantially encapsulated” means that at least greater than 50, 60, 70, 80, 85, 90, 95, 96, 97, 98, 99, 99.9, 99.9 or greater than 99.999% of the pharmaceutical composition or disrupting agent of the invention may be enclosed, surrounded or encased within the delivery agent. “Partial encapsulation” or “partially encapsulated” means that less than 10, 10, 20, 30, 40 50 or less of the pharmaceutical composition or disrupting agent of the invention may be enclosed, surrounded or encased within the delivery agent. Advantageously, encapsulation may be determined by measuring the escape or the activity of the pharmaceutical composition or compound of the invention using fluorescence and/or electron micrograph. For example, at least 1, 5, 10, 20, 30, 40, 50, 60, 70, 80, 85, 90, 95, 96, 97, 98, 99, 99.9, 99.99 or greater than 99.99% of the pharmaceutical composition or disrupting agent of the invention are encapsulated in the delivery agent.

In one embodiment, a disrupting agent comprising a site-specific HNF4α targeting moiety comprising as described herein, may be encapsulated in a therapeutic nanoparticle (e.g., a therapeutic nanoparticle from BIND Therapeutics). Therapeutic nanoparticles may be formulated by methods described herein and known in the art such as, but not limited to, PCT Publication Nos. WO 2010/005740, WO 2010/030763, WO 2010/005721, WO 2010/005723, WO 2012/054923, U.S. Patent Publication Nos. 2201/10262491, 2010/0104645, 2010/0087337, 2010/0068285, 2011/0274759, 2010/0068286, 2012/0288541, 2013/0123351, 2013/0230567, 2013/0236500, 2013/0302433, 2013/0302432, 1013/0280339 and 2013/0251757, and U.S. Pat. Nos. 8,206,747, 8,293,276 8,318,208, 8,318,211, 8,623,417, 8,617,608, 8,613,954, 8,613,951, 8,609,142, 8,603,534 and 8,563,041; the contents of each of which is herein incorporated by reference in their entirety. In another embodiment, therapeutic polymer nanoparticles may be prepared by the methods described in U.S. Patent Publication No. 2012/0140790, herein incorporated by reference in its entirety. As a non-limiting example, the therapeutic nanoparticle may comprise about 4 to about 25 weight percent of a disrupting agent and about 10 to about 99 weight percent of a diblock poly (lactic) acid-poly (ethylene)glycol copolymer comprising poly(lactic) acid as described in US Patent Publication No. 2013/0236500 (Bind), the contents of which are herein incorporated by reference in its entirety. As another non-limiting example, the nanoparticle may comprise about 0.2 to about 35 weight percent of a disrupting agent and about 10 to about 99 weight percent of a diblock poly(lactic) acid-poly(ethylene)glycol copolymer as described in U.S. Patent Publication Nos. 2013/0280339 (Bind) and 2010251757 and U.S. Pat. No. 8,652,528, the contents of each of which are herein incorporated by reference in their entirety.

In one embodiment, a disrupting agent formulated in therapeutic nanoparticles may be administered intramuscularly, intrademrally, or intravenously.

In one embodiment, a disrupting agent formulated in ACCURINS™ nanoparticles may be administered intramuscularly, intrademrally, or intravenously.

In one embodiment, a disrupting agent may be delivered in therapeutic nanoparticles having a high glass transition temperature such as, but not limited to, the nanoparticles described in US Patent Publication Nos. 2014/0030351 and 2011/0294717, the entire contents of each of which are incorporated herein by reference.

In one embodiment, the therapeutic nanoparticle may be formulated for sustained release. As used herein, “sustained release” refers to a pharmaceutical composition or compound that conforms to a release rate over a specific period of time. The period of time may include, but is not limited to, hours, days, weeks, months and years. As a nonlimiting example, the sustained release nanoparticle may comprise a polymer and a disrupting agent of the present invention (see PCT Publication No. WO2010075072 and U.S. Patent Publication Nos. 2010/0216804, 2011/0217377, 2012/0201859, 2013/0243848 and 2013/0243827, each of which is herein incorporated by reference in their entirety).

In one embodiment, a disrupting agent of the invention may be encapsulated in, linked to and/or associated with synthetic nanocarriers. Synthetic nanocarriers include, but are not limited to, those described in PCT Publication. Nos. WO 2010/005740, WO 2010/030763, WO 2012/13501, WO 2012/149252, WO 2012149255, WO 2012149259, WO 2012149265, WO 2012149268, WO 2012149282, WO 2012149301, WO 2012149393, WO 2012149405, WO 2012149411 and WO 2012149454 and US Patent Publication Nos. 20110262491, 20100104645, 20100087337, 20120244222 and US20130236533, and U.S. Pat. No. 8,652,487, the contents of each of which is herein incorporated by reference in their entirety. The synthetic nanocarriers may be formulated using methods known in the art and/or described herein. As a nonlimiting example, the synthetic nanocarriers may be formulated by the methods described in PCT Publication Nos. WO 2010005740, WO 2010030763 and WO 201213501 and US Patent Publication Nos. 20110262491, 20100104645, 20100087337 and 20120244222, each of which is herein incorporated by reference in their entirety. In another embodiment, the synthetic nanocarrier formulations may be lyophilized by methods described in PCT Publication No. WO 2011072218 and U.S. Pat. No. 8,211,473; each of which is herein incorporated by reference in their entirety. In yet another embodiment, formulations of the present invention, including, but not limited to, synthetic nanocarriers, may be lyophilized or reconstituted by the methods described in US Patent Publication No. 20130230568, the contents of which are herein incorporated by reference in its entirety.

In one embodiment, synthetic nanocarriers comprising a disrupting agent may be administered intramuscularly, intrademrally, or intravenously.

In some embodiments, a disrupting agent may be formulated for delivery using smaller LNPs. Such particles may comprise a diameter from below 0.1 μm up to 1000 μm such as, but not limited to, less than 0.1 μm, less than 1.0 μm, less than 5 μm, less than 10 μm, less than 15 μm, less than 20 μm, less than 25 μm, less than 30 μm, less than 35 μm, less than 40 μm, less than 50 μm, less than 55 μm, less than 60 μm, less than 65 μm, less than 70 μm, less than 75 μm, less than 80 μm, less than 85 μm, less than 90 μm, less than 95 μm, less than 100 μm, less than 125 μm, less than 150 μm, less than 175 μm, less than 200 μm, less than 225 μm, less than 250 μm, less than 275 μm, less than 300 μm, less than 325 μm, less than 350 μm, less than 375 μm, less than 400 μm, less than 425 μm, less than 450 μm, less than 475 μm, less than 500 μm, less than 525 μm, less than 550 μm, less than 575 μm, less than 600 μm, less than 625 μm, less than 650 μm, less than 675 μm, less than 700 μm, less than 725 μm, less than 750 μm, less than 775 μm, less than 800 μm, less than 825 μm, less than 850 μm, less than 875 μm, less than 900 μm, less than 925 μm, less than 950 μm, less than 975 μm.

In another embodiment, a disrupting agent may be formulated for delivery using smaller LNPs which may comprise a diameter from about 1 nm to about 100 nm, from about 1 nm to about 10 nm, about 1 nm to about 20 nm, from about 1 nm to about 30 nm, from about 1 nm to about 40 nm, from about 1 nm to about 50 nm, from about 1 nm to about 60 nm, from about 1 nm to about 70 nm, from about 1 nm to about 80 nm, from about 1 nm to about 90 nm, from about 5 nm to about from 100 nm, from about 5 nm to about 10 nm, about 5 nm to about 20 nm, from about 5 nm to about 30 nm, from about 5 nm to about 40 nm, from about 5 nm to about 50 nm, from about 5 nm to about 60 nm, from about 5 nm to about 70 nm, from about 5 nm to about 80 nm, from about 5 nm to about 90 nm, about 10 to about 50 nm, from about 20 to about 50 nm, from about 30 to about 50 nm, from about 40 to about 50 nm, from about 20 to about 60 nm, from about 30 to about 60 nm, from about 40 to about 60 nm, from about 20 to about 70 nm, from about 30 to about 70 nm, from about 40 to about 70 nm, from about 50 to about 70 nm, from about 60 to about 70 nm, from about 20 to about 80 nm, from about 30 to about 80 nm, from about 40 to about 80 nm, from about 50 to about 80 nm, from about 60 to about 80 nm, from about 20 to about 90 nm, from about 30 to about 90 nm, from about 40 to about 90 nm, from about 50 to about 90 nm, from about 60 to about 90 nm and/or from about 70 to about 90 nm.

In one embodiment, a disrupting agent may be formulated in smaller LNPs and may be administered intramuscularly, intrademrally, or intravenously.

In one embodiment, a disrupting agent may be formulated for delivery using the drug encapsulating microspheres described in PCT Patent Publication No. WO 2013063468 or U.S. Pat. No. 8,440,614, each of which is herein incorporated by reference in its entirety. In another aspect, the amino acid, peptide, polypeptide, lipids (APPL) are useful in delivering the disrupting agents of the invention to cells (see PCT Patent Publication No. WO 2013063468, herein incorporated by reference in its entirety).

In one aspect, the lipid nanoparticle may be a limit size lipid nanoparticle described in PCT Patent Publication No. WO 2013059922, herein incorporated by reference in its entirety. The limit size lipid nanoparticle may comprise a lipid bilayer surrounding an aqueous core or a hydrophobic core; where the lipid bilayer may comprise a phospholipid such as, but not limited to, diacylphosphatidylcholine, a diacylphosphatidylethanolamine, a ceramide, a sphingomyelin, a dihydrosphingomyelin, a cephalin, a cerebroside, a C8-C20 fatty acid diacylphophatidylcholine, and I-palmitoyl-2-oIeoyl phosphatidylcholine (POPC). In another aspect the limit size lipid nanoparticle may comprise a polyethylene glycol-lipid such as, but not limited to, DLPEPEG, DMPE-PEG, DPPC-PEG and DSPE-PEG.

In one embodiment, a disrupting agent of the invention may be delivered, localized and/or concentrated in a specific location using the delivery methods described in PCT Patent Publication No. WO 2013063530, the contents of which are herein incorporated by reference in its entirety. As a non-limiting example, a subject may be administered an empty polymeric particle prior to, simultaneously with or after delivering the disrupting agent to the subject. The empty polymeric particle undergoes a change in volume once in contact with the subject and becomes lodged, embedded, immobilized or entrapped at a specific location in the subject.

In one embodiment, a disrupting agent may be formulated in an active substance release system (See e.g., US Patent Publication No. 20130102545, herein incorporated by reference in its entirety). The active substance release system may comprise 1) at least one nanoparticle bonded to an oligonucleotide inhibitor strand which is hybridized with a catalytically active nucleic acid and 2) a compound bonded to at least one substrate molecule bonded to a therapeutically active substance (e.g., a disrupting agent of the invention), where the therapeutically active substance is released by the cleavage of the substrate molecule by the catalytically active nucleic acid.

In one embodiment, the nanoparticles of the present invention may be water soluble nanoparticles such as, but not limited to, those described in PCT Publication No. WO 2013090601, the contents of which are herein incorporated by reference in its entirety. The nanoparticles may be inorganic nanoparticles which have a compact and zwitterionic ligand in order to exhibit good water solubility. The nanoparticles may also have small hydrodynamic diameters (HD), stability with respect to time, pH, and salinity and a low level of non-specific protein binding.

In one embodiment, the nanoparticles of the present invention are stealth nanoparticles or target-specific stealth nanoparticles such as, but not limited to, those described in U.S. Patent Publication Nos. 20130172406 (Bind), US20130251817 (Bind), 2013251816 (Bind) and 20130251766 (Bind), the contents of each of which are herein incorporated by reference in its entirety. The stealth nanoparticles may comprise a diblock copolymer and a chemotherapeutic agent.

These stealth nanoparticles may be made by the methods described in us Patent Publication Nos. 20130172406, 20130251817, 2013251816 and 20130251766, the contents of each of which are herein incorporated by reference in its entirety. As a non-limiting example, the stealth nanoparticles may target cancer cells such as the nanoparticles described in US Patent Publication Nos. 20130172406, 20130251817, 2013251816 and 20130251766, the contents of each of which are herein incorporated by reference in its entirety.

In one embodiment, stealth nanoparticles comprising a disrupting agent of the invention may be administered intramuscularly, intradermally, or intravenously.

In one embodiment, a disrupting agent of the invention may be formulated in and/or delivered in a lipid nanoparticle comprising a plurality of cationic lipids such as, but not limited to, the lipid nanoparticles described in US Patent Publication No. 20130017223, the contents of which are herein incorporated by reference in its entirety. As a non-limiting example, the LNP formulation may comprise a first cationic lipid and a second cationic lipid. As another non-limiting example, the LNP formulation may comprise DLin-MC2-DMA and DLinMC4-DMA. As yet another non-limiting example, the LNP formulation may comprise DLin-MC3-DMA and CI2-200. In one embodiment, the LNP formulations comprising a plurality of cationic lipids (such as, but not limited to, those described in US Patent Publication No. US20130017223, the contents of which are herein incorporated by reference in its entirety) and may be administered intramuscularly, intradermally, or intravenously.

In one embodiment, a disrupting agent as described herein, may be formulated in and/or delivered in a lipid nanoparticle comprising the cationic lipid DLin-MC3-DMA and the neutral lipid DOPE. The lipid nanoparticle may also comprise a PEG based lipid and a cholesterol or antioxidant. These lipid nanoparticle formulations comprising DLin-MC3-DMA and DOPE and a disrupting agent may be administered intramuscularly, intradermally, or intravenously.

In one embodiment, the lipid nanoparticle comprising DLin-MC3-DMA and DOPE may comprise a PEG lipid such as, but not limited to, pentaerythritol PEG ester tetrasuccinimidyl and pentaerythritol PEG ether tetra-thiol, PEGc-DOMG, PEG-DMG (1,2-Dimyristoyl-sn-glycerol, methoxypolyethylene Glycol), PEG-DSG (1,2-Distearoyl-snglycerol, methoxypolyethylene Glycol), PEG-DPG (1,2-Dipalmitoyl-sn-glycerol, methoxypolyethylene glycol), PEG-DSA (PEG coupled to 1,2-distearyloxypropyl-3-amine), PEG-DMA (PEG coupled to 1,2-dimyristyloxypropyl-3-amine, PEG-c-DNA, PEG-c-DMA, PEG-S-DSG, PEG-c-DMA, PEG-DPG, PEG-DMG 2000 and those described herein and/or known in the art.

In one embodiment, the lipid nanoparticle comprising DLin-MC3-DMA and DOPE may include 0.5% to about 3.0%, from about 1.0% to about 3.5%, from about 1.5% to about 4.0%, from about 2.0% to about 4.5%, from about 2.5% to about 5.0% and/or from about 3.0% to about 6.0% of the lipid molar ratio of a PEG lipid.

In one embodiment, the lipid nanoparticle comprising DLin-MC3-DMA and DOPE may include 25.0% cholesterol to about 50.0% cholesterol, from about 30.0% cholesterol to about 45.0% cholesterol, from about 35.0% cholesterol to about 50.0% cholesterol and/or from about 48.5% cholesterol to about 60% cholesterol. In one embodiment, formulations may comprise a percentage of cholesterol selected from the group consisting of 28.5%, 31.5%, 33.5%, 36.5%, 37.0%, 38.5%, 39.0%, 43.5% and 48.5%.

In one embodiment, the lipid nanoparticle comprising DLin-MC3-DMA and DOPE may include 25.0% antioxidant to about 50.0% antioxidant, from about 30.0% antioxidant to about 45.0% antioxidant, from about 35.0% antioxidant to about 50.0% antioxidant and/or from about 48.5% antioxidant to about 60% antioxidant. In one embodiment, formulations may comprise a percentage of antioxidant selected from the group consisting of 28.5%, 31.5%, 33.5%, 36.5%, 37.0%, 38.5%, 39.0%, 43.5% and 48.5%.

The disrupting agent of the invention can be formulated using natural and/or synthetic polymers. Non-limiting examples of polymers which may be used for delivery include, but are not limited to, DYNAMIC POLYCONJUGATE® (Arrowhead Research Corp., Pasadena, Calif.) formulations from MIRUS® Bio (Madison, Wis.) and Roche Madison (Madison, Wis.), PHASERX™ polymer formulations such as, without limitation, SMARTT POLYMER TECHNOLOGY™ (Seattle, Wash.), DMRIIDOPE, poloxamer, VAXFECTIN® adjuvant from Vical (San Diego, Calif.), chitosan, cyclodextrin from Calando Pharmaceuticals (Pasadena, Calif.), dendrimers and poly(lactic-co-glycolic acid) (PLGA) polymers, RONDEL™ (RNAi/Oligonucleotide Nanoparticle Delivery) polymers (Arrowhead Research Corporation, Pasadena, Calif.) and pH responsive co-block polymers such as, but not limited to, PHASERX™ (Seattle, Wash.).

The polymer formulations may permit the sustained or delayed release of a disrupting agent (e.g., following intramuscular, intradermal or subcutaneous injection). The altered release profile of the disrupting agent can result in, for example, translation of an encoded protein over an extended period of time. The polymer formulation may also be used to increase the stability of the disrupting agent. For example, biodegradable polymers have been previously used to protect nucleic acids other than modified mRNA from degradation and been shown to result in sustained release of payloads in vivo (Rozema et al., Proc Natl Acad Sci USA. 2007 104:12982-12887; Sullivan et al., Expert Opin Drug Deliv. 2010 7:1433-1446; Convertine et al., Biomacromolecules. 2010 Oct. 1; Chu et al., Acc Chern Res. 2012 Jan. 13; Manganiello et al., Biomaterials. 2012 33:2301-2309; Benoit et al., Biomacromolecules. 2011 12:2708-2714; Singha et al., Nucleic Acid Ther. 2011 2: 133-147; deFougerolles Hnm Gene Ther. 2008 19:125-132; Schaffert and Wagner, Gene Ther. 2008 16:1131-1138; Chaturvedi et al., Expert Opin Drug Deliv. 2011 8: 1455-1468; Davis, Mol Pharm. 2009 6:659-668; Davis, Nature 201 0464: 1067-1070; each of which is herein incorporated by reference in its entirety).

In one embodiment, the pharmaceutical compositions may be sustained release formulations. In a further embodiment, the sustained release formulations may be for subcutaneous delivery. Sustained release formulations may include, but are not limited to, PLGA microspheres, ethylene vinyl acetate (EVAc), poloxamer, GELSITE® (Nanotherapeutics, Inc. Alachua, Fla.), HYLENEX® (Halozyme Therapeutics, San Diego Calif.), surgical sealants such as fibrinogen polymers (Ethic on Inc. Cornelia, Ga.), TISSELL® (Baxter International, Inc Deerfield, Ill.), PEG-based sealants, and COSEAL® (Baxter International, Inc Deerfield, Ill.).

### B. Vector Encoded Site-Specific HNF4α Disrupting Agents of the Invention

Disrupting agents comprising a site-specific HNF4α targeting moiety, e.g., comprising a nucleic acid molecule, may be expressed from transcription units inserted into DNA or RNA vectors (see, e.g., Couture, A, et al., TIG. (1996), 12:5-10; WO 00/22113, WO 00/22114, and U.S. Pat. No. 6,054,299). In some embodiment, expression is sustained (months or longer), depending upon the specific construct used and the target tissue or cell type. These transgenes can be introduced as a linear construct, a circular plasmid, or a viral vector, which can be an integrating or non-integrating vector. The transgene can also be constructed to permit it to be inherited as an extrachromosomal plasmid (Gassmann, et al., (1995) Proc. Natl. Acad. Sci. USA 92:1292). Different components of the disrupting agent, e.g., gRNA and effector, can be located on separate expression vectors that can be co-introduced (e.g., by transfection or infection) into a target cell. Alternatively, each individual component can be transcribed by promoters both of which are located on the same expression plasmid.

Delivery of a disrupting agent expressing vector can be systemic, such as by intravenous or intramuscular administration, by administration to target cells ex-planted from the patient followed by reintroduction into the patient, or by any other means that allows for introduction into a desired target cell.

In certain embodiment, the nucleic acids described herein or the nucleic acids encoding a protein described herein, e.g., an effector, are incorporated into a vector, e.g., a viral vector.

The individual strand or strands of a disrupting agent comprising a site-specific HNF4α targeting moiety comprising a nucleic acid molecule can be transcribed from a promoter in an expression vector. Where two separate strands are to be expressed to generate, for example, a dsRNA, two separate expression vectors can be co-introduced (e.g., by transfection or infection) into a target cell. Alternatively, each individual strand of a nucleic acid molecule can be transcribed by promoters both of which are located on the same expression plasmid. In one embodiment, a nucleic acid molecule is expressed as inverted repeat polynucleotides joined by a linker polynucleotide sequence such that the nucleic acid molecule has a stem and loop structure.

Expression vectors are generally DNA plasmids or viral vectors. Expression vectors compatible with eukaryotic cells, preferably those compatible with vertebrate cells, can be used to produce recombinant constructs for the expression of a disrupting agent as described herein.

Constructs for the recombinant expression of a disrupting agent will generally require regulatory elements, e.g., promoters, enhancers, etc., to ensure the expression of the disrupting agent in target cells.

Expression of natural or synthetic nucleic acids is typically achieved by operably linking a nucleic acid encoding the nucleic acid of interest to a regulatory region, such as a promoter, and incorporating the construct into an expression vector. The vectors can be suitable for replication and integration in eukaryotes.

Regulatory regions, such as a promoter, suitable for operable linking to a nucleic acid molecules can be operably linked to a regulatory region such as a promoter. can be from any species. Any type of promoter can be operably linked to a nucleic acid sequence. Examples of promoters include, without limitation, tissue-specific promoters, constitutive promoters, and promoters responsive or unresponsive to a particular stimulus (e.g., inducible promoters). Additional promoter elements, e.g., enhancing sequences, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1a (EF-1a). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter.

Further, the present invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

Additional regulatory regions that may be useful in nucleic acid constructs, include, but are not limited to, transcription and translation terminators, initiation sequences, polyadenylation sequences, translation control sequences (e.g., an internal ribosome entry segment, IRES), enhancers, inducible elements, or introns. Such regulatory regions may not be necessary, although they may increase expression by affecting transcription, stability of the mRNA, translational efficiency, or the like. Such regulatory regions can be included in a nucleic acid construct as desired to obtain optimal expression of the nucleic acids in the cell(s). Sufficient expression, however, can sometimes be obtained without such additional elements.

The expression vector to be introduced can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate transcriptional control sequences to enable expression in the host cells. Useful selectable markers include, for example, antibiotic-resistance genes, such as neo and the like. Non-limiting examples of selectable markers include puromycin, ganciclovir, adenosine deaminase (ADA), aminoglycoside phosphotransferase (neo, G418, APH), dihydrofolate reductase (DHFR), hygromycin-B-phosphtransferase, thymidine kinase (TK), and xanthin-guanine phosphoribosyltransferase (XGPRT). Such markers are useful for selecting stable transformants in culture. Other selectable markers include fluorescent polypeptides, such as green fluorescent protein or yellow fluorescent protein.

Signal peptides may also be included and can be used such that an encoded polypeptide is directed to a particular cellular location (e.g., the cell surface).

Reporter genes may be used for identifying potentially transfected cells and for evaluating the functionality of transcriptional control sequences. In general, a reporter gene is a gene that is not present in or expressed by the recipient source and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Other aspects to consider for vectors and constructs are known in the art.

In some embodiments, a vector, e.g., a viral vector comprises a disrupting agent comprising a site-specific HNF4α targeting moiety comprising a nucleic acid molecule.

Viral vector systems which can be utilized with the methods and compositions described herein include, but are not limited to, (a) adenovirus vectors (e.g., an Ad5/F35 vector); (b) retrovirus vectors, including but not limited to lentiviral vectors (including integration competent or integration-defective lentiviral vectors), moloney murine leukemia virus, etc.; (c) adeno-associated virus vectors; (d) herpes simplex virus vectors; (e) SV 40 vectors; (f) polyoma virus vectors; (g) papilloma virus vectors; (h) picornavirus vectors; (i) pox virus vectors such as an orthopox, e.g., vaccinia virus vectors or avipox, e.g. canary pox or fowl pox; and (j) a helper-dependent or gutless adenovirus. Replication-defective viruses can also be advantageous. Different vectors will or will not become incorporated into the cells' genome. The constructs can include viral sequences for transfection, if desired. Alternatively, the construct can be incorporated into vectors capable of episomal replication, e.g. EPV and EBV vectors. See, e.g., U.S. Pat. Nos. 6,534,261; 6,607,882; 6,824,978; 6,933,113; 6,979,539; 7,013,219; and 7,163,824, the entire contents of each of which is incorporated by reference herein.

Vectors, including those derived from retroviruses such as adenoviruses and adeno-associated viruses and lentiviruses, are suitable tools to achieve long-term gene transfer since they allow long-term, stable integration of a transgene and its propagation in daughter cells. Examples of vectors include expression vectors, replication vectors, probe generation vectors, and sequencing vectors. The expression vector may be provided to a cell in the form of a viral vector. Viral vector technology is well known in the art, and described in a variety of virology and molecular biology manuals.

In one embodiment, a suitable viral vector for use in the present invention is an adeno-associated viral vector, such as a recombinant adeno-associate viral vector.

Recombinant adeno-associated virus vectors (rAAV) are gene delivery systems based on the defective and nonpathogenic parvovirus adeno-associated type 2 virus. All vectors are derived from a plasmid that retains only the AAV 145 bp inverted terminal repeats flanking the transgene expression cassette. Efficient gene transfer and stable transgene delivery due to integration into the genomes of the transduced cell are key features for this vector system. (Wagner et al., Lancet 351:9117 1702-3 (1998), Kearns et al., Gene Ther. 9:748-55 (1996)). AAV serotypes, including AAV1, AAV2, AAV3, AAV4, AAV5, AAV6, AAV7, AAV8 and AAV9, can be used in accordance with the present invention.

Replication-deficient recombinant adenoviral vectors (Ad) can be produced at high titer and readily infect a number of different cell types. Most adenovirus vectors are engineered such that a transgene replaces the Ad E1a, E1b, and/or E3 genes; subsequently the replication defective vector is propagated in human 293 cells that supply deleted gene function in trans. Ad vectors can transduce multiple types of tissues in vivo, including nondividing, differentiated cells such as those found in liver, kidney and muscle. Conventional Ad vectors have a large carrying capacity. An example of the use of an Ad vector in a clinical trial involved polynucleotide therapy for antitumor immunization with intramuscular injection (Sterman et al., Hum. Gene Ther. 7:1083-9 (1998)). Additional examples of the use of adenovirus vectors for gene transfer in clinical trials include Rosenecker et al., Infection 24:1 5-10 (1996); Sterman et al., Hum. Gene Ther. 9:7 1083-1089 (1998); Welsh et al., Hum. Gene Ther. 2:205-18 (1995); Alvarez et al., Hum. Gene Ther. 5:597-613 (1997); Topf et al., Gene Ther. 5:507-513 (1998); Sterman et al., Hum. Gene Ther. 7:1083-1089 (1998).

Packaging cells are used to form virus particles that are capable of infecting a host cell. Such cells include 293 cells, which package adenovirus, and ψ2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV

### IV. METHODS OF THE INVENTION

The present invention also provides methods of use of the agents and compositions described herein to modulate expression of hepatocyte nuclear factor 4 alpha-(HNF4α) in a cell. The methods include contacting the cell with a site-specific HNF4α disrupting agent, the disrupting agent comprising a site-specific HNF4α targeting moiety which targets an HNF4α expression control region, and an effector molecule, thereby modulating expression of HNF4α in the cell. The site-specific disrupting agent, the effector, or both the site-specific disrupting agent and the effector may be present in a composition, such as a composition described above. In some embodiments, the site-specific disrupting agent and the effector are present in the same compositions. In other embodiments, the site-specific disrupting agent and the effector are present in different compositions. In some embodiments, the methods of the invention include contacting a cell with two site-specific HNF4α disrupting agents (a first and a second agent). The two site specific HNF4α disrupting agents may be present in the same composition, e.g., pharmaceutical composition, e.g., pharmaceutical composition comprising an LNP, or in separate compositions, e.g., pharmaceutical composition, e.g., pharmaceutical composition comprising an LNP. The cell may be contacted with the first site specific HNF4α disrupting agent at one time and contacted with the second site specific HNF4α disrupting agent at a second time, or the cell may be contacted with both agents at the same time.

As indicated above, in fibrotic liver disease, HNF4α is dysregulated and, as a result, gene expression in its network declines significantly or stops. As reported by Guzman-Lepe (Guzman-Lepe J, et al. Liver-enriched transcription factor expression relates to chronic hepatic failure in humans. Hepatol Commun. 2018; 2(5):582-594. doi:10.1002/hep4.1172), HNF4α expression was down-regulated and correlated well with the extent of liver dysfunction (P=0.001), stage of fibrosis (P=0.0005), and serum levels of total bilirubin (P=0.009; r=0.35), albumin (P<0.001; r=0.52), and prothrombin time activity (P=0.002; r=0.41). HNF4α expression also correlated with CYP3A4, ornithine transcarbamylase (OTC), and F7 as well as CDH1 RNA levels. This dysregulation of the network contributes to the pathology of liver failure in the organ itself, and to co-morbidities throughout the patient. In addition to a repression of proteins associated with healthy liver (e.g. albumin and CYP3A enzyme production), proteins that contribute to the production of fibrosis are activated, including COL1a1 and αSMA.

Proof of principle that increased expression of HNF4α can revert senescent and irreversibly dysfunctional hepatocytes from terminal rodent livers to normal function has been established in several studies (Nishikawa, supra; Scholten D, Trebicka J, Liedtke C, Weiskirchen R. The carbon tetrachloride model in mice. Lab Anim. 2015; 49(1 Suppl):4-11. doi:10.1177/0023677215571192; Varga J, Brenner D A, Phan S H, eds. Fibrosis Research: Methods and Protocols. Humana Press; 2005. doi:10.1385/1592599400). Interestingly, as reported by Nishikawa et al. (Nishikawa, supra), reversal of the distorted extracellular matrix is not absolutely required to reverse hepatic failure in degenerative liver disease, as only minimal resolution of fibrosis was found by histology two weeks after forced re-expression of HNF4α, well after improvement in hepatic function was documented. Significant improvement in histology, however, was observed at 100 days. In addition, long-term correction took place despite that forced re-expression generated only 0.01% of the endogenous level of HNF4α. Thus, improvement in hepatic function may only require increasing expression of HNF4α in a relatively modest number of hepatocytes in end-stage degenerative disease.

Recently, Huang et al. (Huang K-W, et al. Liver Activation of Hepatocellular Nuclear Factor-4α by Small Activating RNA Rescues Dyslipidemia and Improves Metabolic Profile. Mol Ther Nucleic Acids. 2019; 19:361-370. doi:10.1016/j.omtn.2019.10.044) also observed that stimulating HNF4α expression with a small-activating RNA in a rat model of non-alcoholic fatty liver disease (NAFLD) restored metabolic regulation and improved lipid profile.

As demonstrated in the examples below, one embodiment of the invention is to increase the expression of the HNF4α gene by delivering an engineered transcription factor to the gene that is pathologically dysregulated. The transcriptional activator, VPR (Chavez A, et al. Highly efficient Cas9-mediated transcriptional programming. Nat Methods. 2015; 12(4):326-328. doi:10.1038/nmeth.3312), is a concatemer of the HSV transcriptional activator VP16, nuclear factor NF-kappa-B p65 subunit, and the EBV R transactivator. Each of these transcription factors is capable, individually, of attracting the cellular machinery of transcription, resulting in an upregulation of RNA production from the target locus. As a group, their synergistic cooperation results in physiologic or supra-physiologic expression of a target gene.

Expression of HNF4α may be enhanced or reduced as compared to, for example, a cell that was not contacted with the site-specific HNF4α disrupting agent. Modulation in gene expression can be assessed by any methods known in the art. For example, a modulation in the expression may be determined by determining the mRNA expression level of a gene, e.g., in a cell, a plurality of cells, and/or a tissue sample, using methods routine to one of ordinary skill in the art, e.g., northern blotting, qRT-PCR; by determining the protein level of a gene using methods routine to one of ordinary skill in the art, such as western blotting, immunological techniques.

The term “reduced” in the context of the level of HNF4α gene expression or HNF4α protein production in a subject, or a disease marker or symptom refers to a statistically significant decrease in such level. The decrease can be, for example, at least 20%, 25%, 30%, 35%, 40%, 45%, 50%, 55%, 60%, 65%, 70%, 75%, 80%, 85%, 90%, or 95%, or below the level of detection for the detection method. In certain embodiments, the expression of the target is normalized, i.e., decreased towards or to a level accepted as within the range of normal for an individual without such disorder. As used here, “lower” in a subject can refer to lowering of gene expression or protein production in a cell in a subject does not require lowering of expression in all cells or tissues of a subject. For example, as used herein, lowering in a subject can include lowering of gene expression or protein production in the liver of a subject.

The term “reduced” can also be used in association with normalizing a symptom of a disease or condition, i.e. decreasing the difference between a level in a subject suffering from an HNF4α-associated disease towards or to a level in a normal subject not suffering from an HNF4α-associated disease. As used herein, if a disease is associated with an elevated value for a symptom, “normal” is considered to be the upper limit of normal. If a disease is associated with a decreased value for a symptom, “normal” is considered to be the lower limit of normal.

The term “enhanced” in the context of the level of HNF4α gene expression or HNF4α protein production in a subject, or a disease marker or symptom refers to a statistically significant increase in such level. The increase can be, for example, at least 20%, 25%, 30%, 35%, 40%, 45%, 50%, 55%, 60%, 65%, 70%, 75%, 80%, 85%, 90%, or 95%, or above the level of detection for the detection method. In certain embodiments, the expression of the target is normalized, i.e., increase towards or to a level accepted as within the range of normal for an individual without such disorder. As used here, “higher” in a subject can refer to increasing gene expression or protein production in a cell in a subject does not require increasing expression in all cells or tissues of a subject. For example, as used herein, increasing in a subject can include increasing gene expression or protein production in the liver of a subject.

The term “enhanced” can also be used in association with normalizing a symptom of a disease or condition, i.e. increasing the difference between a level in a subject suffering from an HNF4α-associated disease towards or to a level in a normal subject not suffering from an HNF4α-associated disease. As used herein, if a disease is associated with an elevated value for a symptom, “normal” is considered to be the upper limit of normal. If a disease is associated with a decreased value for a symptom, “normal” is considered to be the lower limit of normal.

In some embodiments, a suitable cell for use in the methods of the invention is a mammalian cell. In some embodiments, the cell is a somatic cell. In some embodiments, the cell is a primary cell. For example, in some embodiments, the cell is a mammalian somatic cell. In some embodiments, the mammalian somatic cell is a primary cell. In some embodiments, the mammalian somatic cell is a non-embryonic cell.

The step of contacting may be performed in vitro, in vivo (i.e., the cell may be within a subject), or ex vivo. In some embodiments, contacting a cell is performed ex vivo and the methods further include, prior to the step of contacting, a step of removing the cell (e.g., a mammalian cell) from a subject. In some embodiments, the methods further comprise, after the step of contacting, a step of (b) administering the cell (e.g., mammalian cells) to a subject.

The in vivo methods of the invention may include administering to a subject an agent or composition of the invention.

The term “subject,” as used herein refers to an organism, for example, a mammal (e.g., a human, a non-human mammal, a non-human primate, a primate, a laboratory animal, a mouse, a rat, a hamster, a gerbil, a cat, or a dog). In some embodiments a human subject is an adult, adolescent, or pediatric subject. In some embodiments, a subject had a disease or a condition. In some embodiments, the subject is suffering from a disease, disorder or condition, e.g., a disease, disorder or condition that can be treated as provided herein. In some embodiments, a subject is susceptible to a disease, disorder, or condition; in some embodiments, a susceptible subject is predisposed to and/or shows an increased risk (as compared to the average risk observed in a reference subject or population) of developing the disease, disorder or condition. In some embodiments, a subject displays one or more symptoms of a disease, disorder or condition. In some embodiments, a subject does not display a particular symptom (e.g., clinical manifestation of disease) or characteristic of a disease, disorder, or condition. In some embodiments, a subject does not display any symptom or characteristic of a disease, disorder, or condition. In some embodiments, a subject is a patient. In some embodiments, a subject is an individual to whom diagnosis and/or therapy is and/or has been administered.

Subjects that would benefit from the methods of the invention include subjects having an “HNF4α-associated disease” or a subject at risk of an “HNF4α-associated disease.”

Thus, the present invention further provides methods of treatment of a subject in need thereof. The treatment methods of the invention include administering an agent or composition of the invention to a subject, e.g., a subject that would benefit from a modulation of HNF4α expression, such as a subject having an HNF4α-associated disease, in a therapeutically effective amount. In some embodiments, the methods of the invention include the subject may be administered two site-specific HNF4α disrupting agents (a first and a second agent). The two site specific HNF4α disrupting agents may be present in the same composition, e.g., pharmaceutical composition, e.g., pharmaceutical composition comprising an LNP, or in separate compositions, e.g., pharmaceutical composition, e.g., pharmaceutical composition comprising an LNP. The subject may be administered the first site specific HNF4α disrupting agent at one time and administered the second site specific HNF4α disrupting agent at a second time, or the subject may be administered both agents at the same time.

In addition, the present invention provides methods for preventing at least one symptom in a subject that would benefit from a modulation of HNF4α expression, such as a subject having an HNF4α-associated disease, by administering to the subject an agent or composition of the invention in a prophylactically effective amount.

“Therapeutically effective amount,” as used herein, is intended to include the amount of an agent or composition that, when administered to a patient for treating a subject having a HNF4α-associated disease, is sufficient to effect treatment of the disease (e.g., by diminishing, ameliorating, or maintaining the existing disease or one or more symptoms of disease or its related comorbidities).

The “therapeutically effective amount” may vary depending on the agent or composition, how it is administered, the disease and its severity and the history, age, weight, family history, genetic makeup, stage of pathological processes mediated by HNF4α gene expression, the types of preceding or concomitant treatments, if any, and other individual characteristics of the patient to be treated.

“Prophylactically effective amount,” as used herein, is intended to include the amount of an agent or composition that, when administered to a subject who does not yet experience or display symptoms of an HNF4α-associated disease, but who may be predisposed to an HNF4α-associated disease, is sufficient to prevent or delay the development or progression of the disease or one or more symptoms of the disease for a clinically significant period of time. The “prophylactically effective amount” may vary depending on the agent or composition, how it is administered, the degree of risk of disease, and the history, age, weight, family history, genetic makeup, the types of preceding or concomitant treatments, if any, and other individual characteristics of the patient to be treated.

As used herein, “prevention” or “preventing,” when used in reference to a disease, disorder or condition thereof, that would benefit from a reduction in expression of an HNF4α gene or production of HNF4α protein, refers to a reduction in the likelihood that a subject will develop a symptom associated with such a disease, disorder, or condition, e.g., a sign or symptom of HNF4α gene expression or HNF4α activity.

A “therapeutically-effective amount” or “prophylactically effective amount” also includes an amount of an agent or composition that produces some desired local or systemic effect at a reasonable benefit/risk ratio applicable to any treatment. Agents and compositions employed in the methods of the present invention may be administered in a sufficient amount to produce a reasonable benefit/risk ratio applicable to such treatment. In some embodiments, a therapeutically effective amount or prophylactically effect amount tis administered in a single dose; in some embodiments, multiple unit doses are required to deliver a therapeutically or prophylactically effective amount.

As used herein, the phrase “symptoms are reduced” may be used when one or more symptoms of a particular disease, disorder or condition is reduced in magnitude (e.g., intensity, severity, etc.) and/or frequency. In some embodiments, a delay in the onset of a particular symptom is considered one form of reducing the frequency of that symptom.

When the subject to be treated is a mammal such as a human, the composition can be administered by any means known in the art including, but not limited to oral, intraperitoneal, or parenteral routes, including intracranial (e.g., intraventricular, intraparenchymal, and intrathecal), intravenous, intramuscular, subcutaneous, transdermal, airway (aerosol), nasal, rectal, and topical (including buccal and sublingual) administration. In certain embodiments, the compositions are administered by intravenous infusion or injection. In certain embodiments, the compositions are administered by subcutaneous injection.

As used herein, the term “HNF4α-associated disease,” is a disease or disorder that is caused by, or associated with HNF4α gene expression or HNF4α protein production. The term “HNF4α-associated disease” includes a disease, disorder or condition that would benefit from a decrease in HNF4α gene expression, replication, or protein activity. Non-limiting examples of HNF4α-associated diseases include, for example, liver disease (e.g., fatty liver, steatohepatitis including non-alcoholic steatohepatitis (NASH)), inflammatory bowel disease (IBD), hepatocellular carcinoma, MODY I, polycystic kidney disease, dyslipidemia (e.g., hyperlipidemia, high LDL cholesterol, low HDL cholesterol, hypertriglyceridemia, postprandial hypertriglyceridemia), disorders of glycemic control (e.g., insulin resistance not related to immune response to insulin, type 2 diabetes), cardiovascular disease (e.g., hypertension, endothelial cell dysfunction), kidney disease (e.g., acute kidney disorder, tubular dysfunction, proinflammatory changes to the proximal tubules, chronic kidney disease), metabolic syndrome, disease of lipid deposition or dysfunction (e.g., adipocyte dysfunction, visceral adipose deposition, obesity), disease of elevated uric acid (e.g., hyperuricemia, gout), and eating disorders such as excessive sugar craving. Details regarding signs and symptoms of the various diseases or conditions are well known in the art.

In one embodiment, the HNF4α-associated disease is selected from the group consisting of fatty liver (steatosis), nonalcoholic steatohepatitis (NASH), cirrhosis of the liver, accumulation of fat in the liver, inflammation of the liver, hepatocellular necrosis, liver fibrosis, and nonalcoholic fatty liver disease (NAFLD), polycystic kidney disease, inflammatory bowel disease (IBD), and MODY I.

Administration of the agents or compositions according to the methods of the invention may result in a reduction of the severity, signs, symptoms, or markers of an HNF4α-associated disease or disorder in a patient with an HNF4α-associated disease or disorder. By “reduction” in this context is meant a statistically significant decrease in such level. The reduction (absolute reduction or reduction of the difference between the elevated level in the subject and a normal level) can be, for example, at least about 20%, 25%, 30%, 35%, 40%, 45%, 50%, 55%, 60%, 65%, 70%, 75%, 80%, 85%, 90%, or 95%, or to below the level of detection of the assay used.

Administration of the agents or compositions according to the methods of the invention may stably or transiently modulating expression of a target gene. In some embodiments, a modulation of expression persists for at least about 1 hr to about 30 days, or at least about 2 hrs, 6 hrs, 12 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or longer or any time therebetween. In some other embodiments, a modulation of expression persists for no more than about 30 mins to about 7 days, or no more than about 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 24 hrs, 36 hrs, 48 hrs, 60 hrs, 72 hrs, 4 days, 5 days, 6 days, 7 days, or any time therebetween.

The agents or compositions may be administered once to the subject or, alternatively, multiple administrations may be performed over a period of time. For example, two, three, four, five, or more administrations may be given to the subject during one treatment or over a period of time. In some embodiments, six, eight, ten, 12, 15 or 20 or more administrations may be given to the subject during one treatment or over a period of time as a treatment regimen.

In some embodiments, administrations may be given as needed, e.g., for as long as symptoms associated with the disease, disorder or condition persist. In some embodiments, repeated administrations may be indicated for the remainder of the subject's life. Treatment periods may vary and could be, e.g., one day, two days, three days, one week, two weeks, one month, two months, three months, six months, a year, or longer.

Efficacy of treatment or prevention of disease can be assessed, for example by measuring disease progression, disease remission, symptom severity, reduction in pain, quality of life, dose of a medication required to sustain a treatment effect, level of a disease marker, or any other measurable parameter appropriate for a given disease being treated or targeted for prevention. It is well within the ability of one skilled in the art to monitor efficacy of treatment or prevention by measuring any one of such parameters, or any combination of parameters. As discussed herein, the specific parameters to be measured depend on the HNF4α-associated disease that the subject is suffering from.

Comparisons of the later readings with the initial readings provide a physician an indication of whether the treatment is effective. It is well within the ability of one skilled in the art to monitor efficacy of treatment or prevention by measuring any one of such parameters, or any combination of parameters. In connection with the administration of an agent or composition, “effective against” a HNF4α-associated disorder indicates that administration in a clinically appropriate manner results in a beneficial effect for at least a statistically significant fraction of patients, such as a improvement of symptoms, a cure, a reduction in disease, extension of life, improvement in quality of life, or other effect generally recognized as positive by medical doctors familiar with treating HNF4α-associated disorders.

A treatment or preventive effect is evident when there is a statistically significant improvement in one or more parameters of disease status, or by a failure to worsen or to develop symptoms where they would otherwise be anticipated. As an example, a favorable change of at least 10% in a measurable parameter of disease, and preferably at least 20%, 30%, 40%, 50% or more can be indicative of effective treatment. Efficacy for a given agent or composition can also be judged using an experimental animal model for the given disease as known in the art. When using an experimental animal model, efficacy of treatment is evidenced when a statistically significant reduction in a marker or symptom is observed.

Alternatively, the efficacy can be measured by a reduction in the severity of disease as determined by one skilled in the art of diagnosis based on a clinically accepted disease severity grading scale. Any positive change resulting in e.g., lessening of severity of disease measured using the appropriate scale, represents adequate treatment using an agent or composition as described herein.

As used herein, the terms “treating” or “treatment” refer to a beneficial or desired result including, but not limited to, alleviation or amelioration of one or more signs or symptoms associated with HNF4α gene expression or HNF4α protein production. “Treatment” can also mean prolonging survival as compared to expected survival in the absence of treatment.

The present invention is next described by means of the following examples. However, the use of these and other examples anywhere in the specification is illustrative only, and in no way limits the scope and meaning of the invention or of any exemplified form. The invention is not limited to any particular preferred embodiments described herein. Many modifications and variations of the invention may be apparent to those skilled in the art and can be made without departing from its spirit and scope. The contents of all references, patents and published patent applications cited throughout this application, including the figures and informal sequence listing, are incorporated herein by reference.

### EXAMPLES

### Example 1. Modulation of HNF4α Expression

This example describes silencing of HNF4α expression with a site-specific HNF4α disrupting agent, comprising a site-specific HNF4α targeting moiety which targets an HNF4α expression control region, i.e., a guide RNA, and an effector comprising a fusion molecule comprising dCAS9 and KRAB.

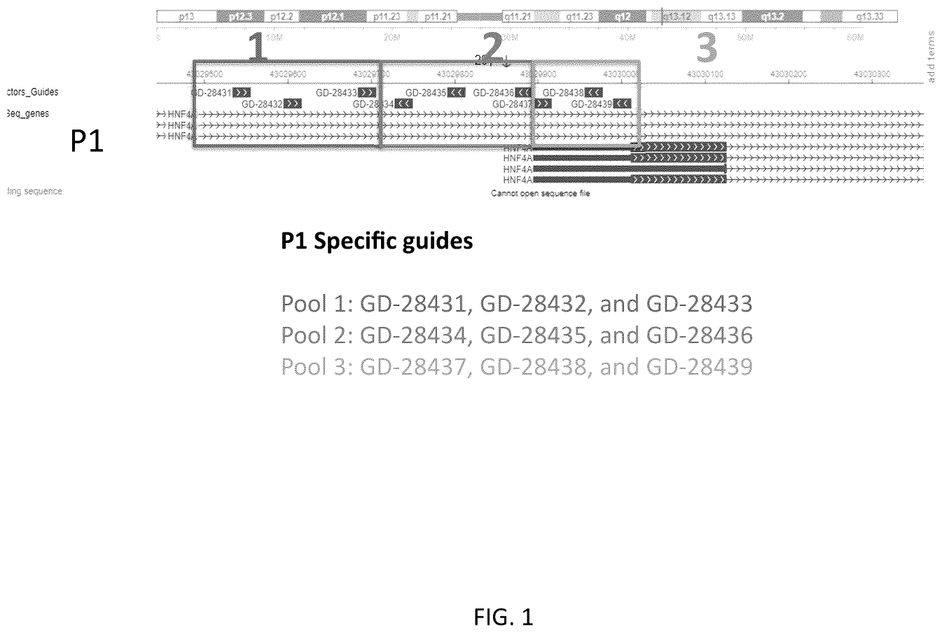

Guide RNAs were designed to site-specifically target the transcriptional control region comprising promoter 1 (near the transcriptional start site) of the HNF4α gene (see, e.g., FIG. 1) and synthesized according to standard methods for oligonucleotide synthesis. The nucleotide sequences of the guide RNAs are provided in Table 2, below. Exemplary guide RNAs designed to site-specifically target the transcriptional control region comprising promoter 2 (near the transcriptional start site) of the HNF4α gene were also designed and are provided in Table 3, below. Additional exemplary guide RNAs designed to site-specifically target the transcriptional control region comprising promoter 2 (near the transcriptional start site) of the HNF4α gene were also designed and are provided in Table 9, below. Table 4, below, includes the unmodified nucleotide sequences, reverse complement nucleotide sequences, and chromosomal coordinates of the targeting portion of the guide RNAs in Tables 2 and 3.

HepG2 cells were seeded in 96-well plate (at a density of 3×10E4 cells/well) in appropriate media 24 hours prior to transfection. Cells were transfected with SSOP Lipid Nano-Particles (LNPs) containing mRNA encoding fusion proteins for dCAS9-KRAB (MR_28122) and guide RNAs (sgRNA) according to standard methods (see, e.g., Akita, et al. (Advanced Healthcare Materials (2013) 2:8:1120-1125).

LNP formulations prepared using individual sgRNAs or Pools of sgRNAs (see, FIG. 1), were added to the cells for a final concentration of 5.0, 2.5, 1.25, or 0.625 g/ml. The experiment was ended after 72 hours by lysing/freezing in RLT buffer for downstream mRNA purification or treatment with the Cell Titer Glo 2 reagent to quantify final cell number. qPCR was performed using HNF4α and ACTB probes to quantify relative HNF4α RNA transcription.

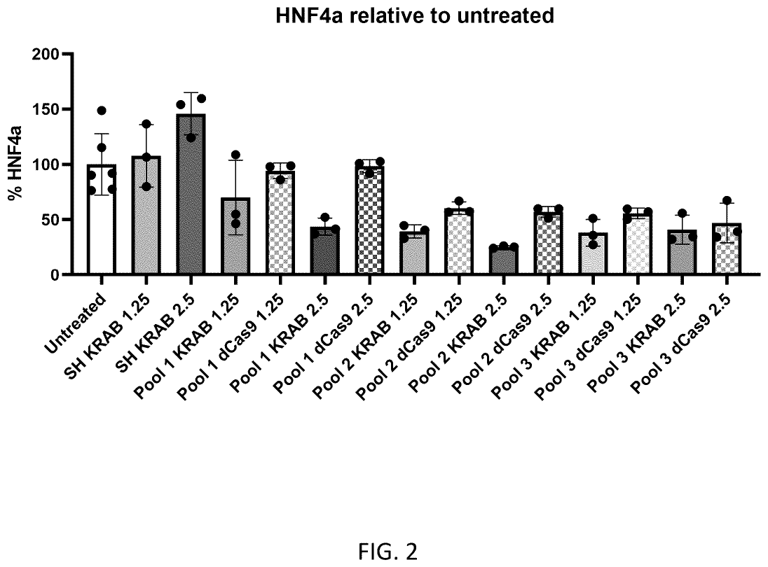

FIG. 2 demonstrates that Pool 1 or Pool 2 guides in combination with the effector dCAS-KRAB inhibited HNF4α expression in a dose dependent manner relative to dCAS alone, SH-KRAB, and untreated. dCAS alone does affect transcription in some cases, and without wishing to be bound by theory, the inhibition may be the result of dCAS interference with polymerase binding.

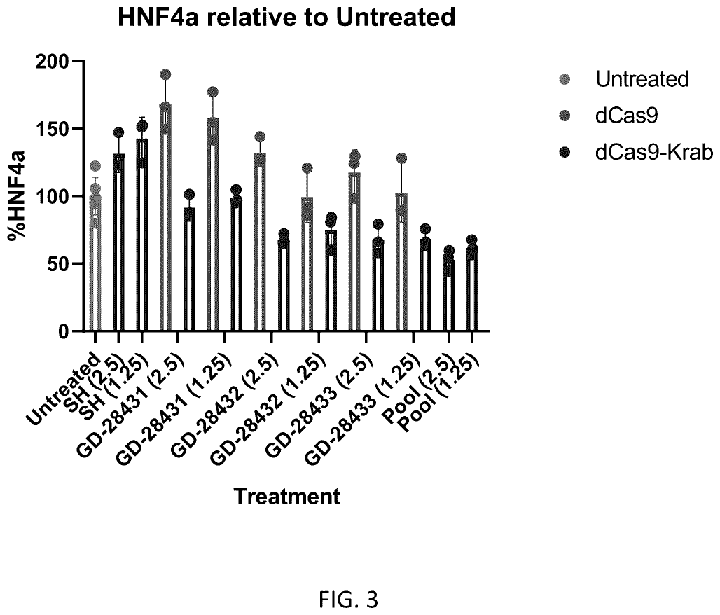

In addition, and as demonstrated in FIG. 3, Pool 1 at the highest concentration has the strongest silencing activity and guide RNAs GD-28432 and GD-28433 each have strong silencing activity alone, even at the lowest concentration.

### Example 2. Modulation of HNF4α Expression

This example describes activation of HNF4α expression with a site-specific HNF4α disrupting agent, comprising a site-specific HNF4α targeting moiety which targets an HNF4α expression control region, i.e., a guide RNA, and an effector comprising a fusion molecule comprising dCAS9 (a non-editing Cas9 protein) and VPR.

Guide RNAs were designed to site-specifically target the transcriptional control region comprising promoter 1 (near the transcriptional start site) of the HNF4α gene (see, e.g., FIG. 1) and were synthesized according to standard methods for oligonucleotide synthesis. The nucleotide sequences of the guide RNAs are provided in Table 2, below. Exemplary guide RNAs designed to site-specifically target the transcriptional control region comprising promoter 2 (near the transcriptional start site) of the HNF4α gene were also designed and are provided in Table 3, below. Additional exemplary guide RNAs designed to site-specifically target the transcriptional control region comprising promoter 2 (near the transcriptional start site) of the HNF4α gene were also designed and are provided in Table 9, below. Table 4, below, includes the unmodified nucleotide sequences, reverse complement nucleotide sequences, and chromosomal coordinates of the guide RNAs in Tables 2 and 3.

LX-2, A549, and HepG2 cells were seeded in 96-well plates (at a density of 1×10E4 cells/well) in appropriate media 24 hours prior to transfection. LX-2 cells normally do not express HNF4α; HepG2 cells, liver cancer cell line cells, normally express more HNF4α that normal non-cancerous liver cells.

Cells were transfected with mRNA encoding fusion proteins for dCAS9-VPR (MR_28196) and guide RNAs (sgRNA). Individual sgRNAs or pools of sgRNAs were prepared using the Lipofectamine MessengerMAX Transfection protocol as described by the manufacturer. Untreated HepG2 cells were also included in the experiment as an internal control (comparator).

Briefly, 2.5 μl lipofectamine per g of RNA was mixed with OptiMEM media and incubated for 10 minutes at room temperature (RT), and then added to cells in a 1:10 ratio of the media volume to the cells. Lipofectmine media mix was combined with RNA for a total volume of 120 μl at a final concentration of 100 g/ml. This mix was incubated at RT for 5 minutes and added to the cells for a final concentration of 5.0, 2.5, 1.25, 0.625 g/ml.

The experiment was ended after 48 hours by lysing/freezing in RLT buffer for downstream mRNA purification or treatment with the Cell Titer Glo 2 reagent to quantify final cell number. Briefly, the plate for RNA extraction was washed three times with PBS, following which 150 μL RLT buffer was added to each well. The plate was then frozen at −80° C. for later RNA processing. RNA was extracted following thawing of the plates, using the Qiagen RNeasy 96-well kit. RNA was quantified using Ribogreen. Reverse transcription and qPCR were carried out following the protocol for Absolute Quantitation for mRNA Expression by RT-qPCR.

The standard curves for HNF4α and β-actin (ACTB) were prepared as follows: HNF4α and ACTB gene block stocks (a synthesized reference copy of target cDNA) were prepared in nuclease-free water to a concentration of 10 mg/mL. A mixture 0.5 mg/mL of each gene block stock was prepared by combining 5 μL of each individual gene block stock and 40 μL H2O to a final volume of 50 uL. This mixture was then serially diluted (10-fold dilutions) 8 times. Two microliter (2 μL) of each standard curve dilution was used as the cDNA for the standard curve to which was added 8 μL of Taqman Master Mix with probes. The standard curve was set up in duplicate Wells were analyzed in technical triplicates.

RNA was extracted and HNF4α mRNA levels were determined by qPCR.

qPCR was performed using HNF4α and ACTB probes to quantify relative HNF4α RNA transcription. HepG2 HNF4α RNA expression was measured as a positive control.

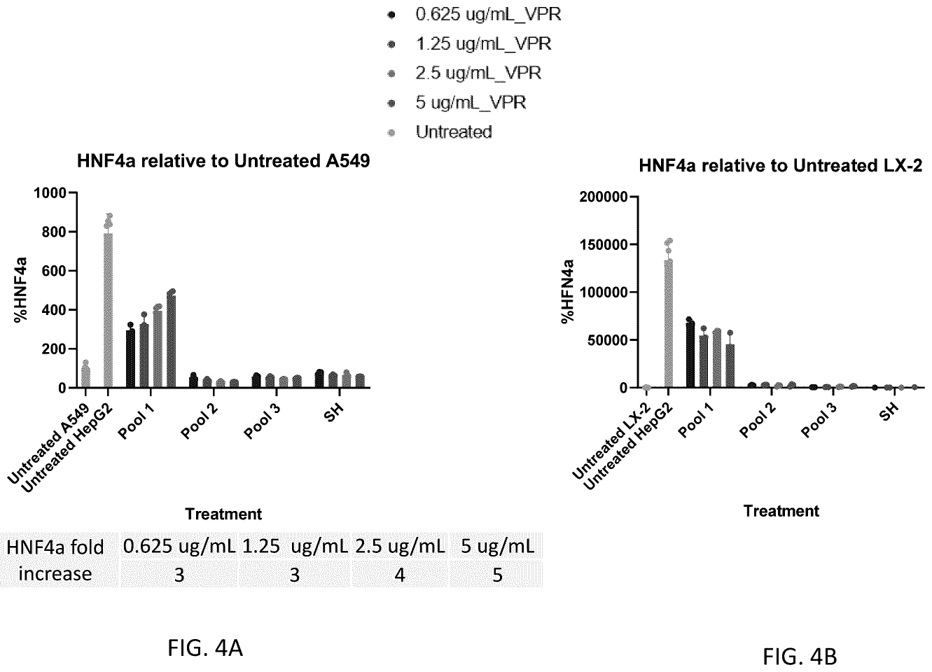

FIGS. 4A and 4B demonstrate that Pool 1 guides in combination with the effector dCAS-VPR show strong activation of HNF4α expression in both A549 cells and LX-2 cells in a dose dependent manner relative to SH-VPR, and untreated.

Delivering dCAS9-P300 using Lipofectamine MessengerMAX Transfection also upregulates HNF4α, but significantly less than dCAS-VPR (data not shown).

Similar experiments were conducted to evaluate the effect of dCas9 fusion proteins comprising dCas9-VPR and guides targeting the promoter region of HNF4α using Dlin-MC3-DMA (MC3), an LNP formulated for in vivo delivery. Using MC3-LNP, mRNA for dCas9-VPR and individual and pooled sgRNAs (Pool 1) were delivered to cells in culture. RNA was extracted and HNF4α mRNA levels were determined by qPCR as described above. Untreated HepG2 cells were included in the experiment as an internal control (comparitor).

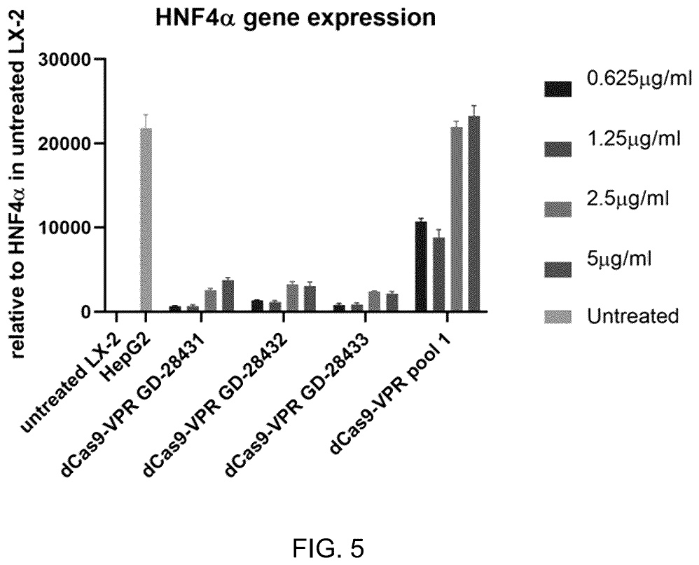

FIG. 5 demonstrates that, in this experiment, the MC3 LNPs are more efficient at transfecting LX-2 cells than Messenger Max in comparison to HNF4α levels to HepG2 (internal control). The individual sgRNAs targeting the HNF4α promoter show upregulation but significantly less than the pooled guides.

Activation of HNF4α in HepG2 Cells using MC3-LNP mediated delivery was also evaluated. HepG2 cells express a basal level of HNF4α that is higher than normal cells. Supraphysiological expression may facilitate greater durability of efficacy.

Using MC3-LNP, mRNA for dCas9-VPR and pooled sgRNAs were delivered to HepG2 cells in culture. A dCas9-VPR control with a control, SH (safe harbor non-targeting control) guide, was also included. HNF4α mRNA levels were determined by qPCR.

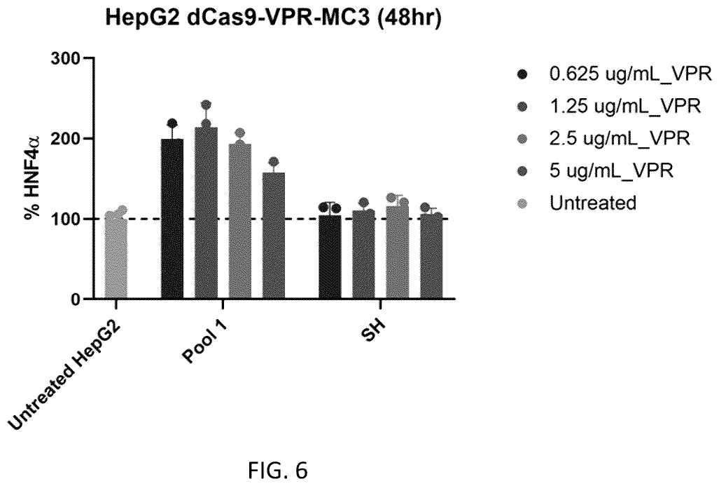

FIG. 6 demonstrates that MC3-LNP formulations induced over-expression of HNF4α in HepG2 cells. Expression levels of up to about 200% of control levels were observed. Such expression level were tolerated in cells in culture. The result of the SH control demonstrates that upregulation of HNF4α is specifically due to the promoter targeted delivery of the activating effector.

To investigate whether the HNF4α protein induced by dCas9-VPR Pool 1 was localized to the nucleus of the cell, LX-2 cells were transfected with mRNA for dCas9-VPR and pooled sgRNAs (Pool 1). In order to visualize cells, treated LX-2 cells were fixed and permeabilized. Cells were grown in 96 well plates with black walls and a clear bottom. Media was removed. All incubations were at room temperature (RT) and stationary. All solutions were made with 10×PBS stock solution.

Media was removed and cells were washed 2 times with 1×PBS. To wash the cells, 100 μl of 1×PBS was added, and removed. The wash was repeated twice. One hundred microliter (100 μl) of 4% PFA in 1×PBS was added and incubated for 15 minutes at RT. Cells were washed 3 times with 1×PBS, with 5 minutes incubation between each wash. Cells were then incubated in 100 μl of 0.1% Triton X 100 in 1×PBS for 15 minutes at RT. One hundred microliter (100 μl) of 10% normal goat serum in 1×PBS with 0.1% Triton X 100 was added and kept at 4 C until further use.

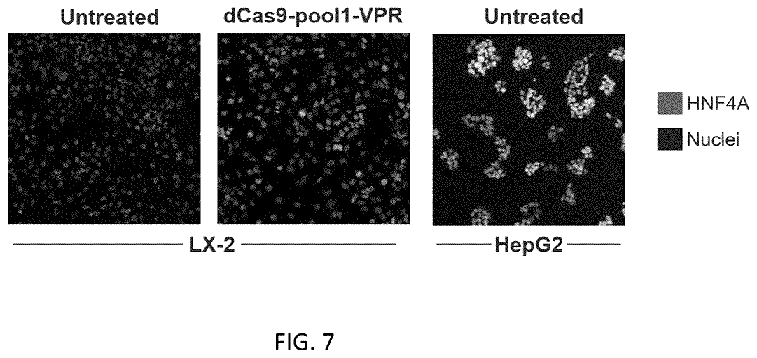

The fixed and permeabilized cells were stained with anti-HNF4α antibody and DAPL. FIG. 7 shows that the HNF4α protein induced by dCas9-VPR Pool 1 can be detected and travels to the nucleus.

TABLE 2
Site-Specific HNF4α Promoter 1 Targeting Moieties (sgRNA)- The first 20 nucleotides
in each moiety below comprise the targeting portion of the moiety.

| | SEQ | |
|---|---|---|
| | ID | |
| Identifier | NO. | Modified Nucleotide Sequence 5′ to 3′ |
| GD-28431 | 68 | mAs; mUs; mUs; rG; rA; rA; rU; rU; rA; rG; rG; rG; rG; rA; rU; rC; rU; rC; rG; rG; |
| | | rG; rU; rU; rU; rU; rA; rG; rA; rG; rC; rU; rA; rG; rA; rA; rA; rU; rA; rG; rC; rA; |
| | | rA; rG; rU; rU; rA; rA; rA; rA; rU; rA; rA; rG; rG; rC; rU; rA; rG; rU; rC; rC; rG; |
| | | rU; rU; rA; rU; rC; rA; rA; rC; rU; rU; rG; rA; rA; rA; rA; rA; rG; rU; rG; rG; rC; |
| | | rA; rC; rC; rG; rA; rG; rU; rC; rG; rG; rU; rG; rC; rUs; mUs; mUs; mU |
| GD-28432 | 69 | mGs; mAs; mCs; rU; rU; rG; rG; rG; rG; rU; rG; rA; rC; rA; rA; rU; rG; rG; rC; rU; |
| | | rG; rU; rU; rU; rU; rA; rG; rA; rG; rC; rU; rA; rG; rA; rA; rA; rU; rA; rG; rC; |
| | | rA; rA; rG; rU; rU; rA; rA; rA; rA; rU; rA; rA; rG; rG; rC; rU; rA; rG; rU; rC; |
| | | rC; rG; rU; rU; rA; rU; rC; rA; rA; rC; rU; rU; rG; rA; rA; rA; rA; rA; rG; rU; |
| | | rG; rG; rC; rA; rC; rC; rG; rA; rG; rU; rC; rG; rG; rU; rG; rC; rUs; mUs; mUs; mU |
| GD-28433 | 70 | mAs; mAs; mCs; rU; rG; rA; rA; rC; rA; rU; rC; rG; rG; rU; rG; rA; rG; rU; rU; rA; |
| | | rG; rU; rU; rU; rU; rA; rG; rA; rG; rC; rU; rA; rG; rA; rA; rA; rU; rA; rG; rC; rA; |
| | | rA; rG; rU; rU; rA; rA; rA; rA; rU; rA; rA; rG; rG; rC; rU; rA; rG; rU; rC; rC; rG; |
| | | rU; rU; rA; rU; rC; rA; rA; rC; rU; rU; rG; rA; rA; rA; rA; rA; rG; rU; rG; rG; rC; |
| | | rA; rC; rC; rG; rA; rG; rU; rC; rG; rG; rU; rG; rC; rUs; mUs; mUs; mU |
| GD-28434 | 71 | mUs; mGs; mGs; rU; rU; rU; rC; rU; rG; rG; rC; rU; rG; rA; rC; rA; rC; rC; rC; rG; |
| | | rG; rU; rU; rU; rU; rA; rG; rA; rG; rC; rU; rA; rG; rA; rA; rA; rU; rA; rG; rC; rA; |
| | | rA; rG; rU; rU; rA; rA; rA; rA; rU; rA; rA; rG; rG; rC; rU; rA; rG; rU; rC; rC; rG; |
| | | rU; rU; rA; rU; rC; rA; rA; rC; rU; rU; rG; rA; rA; rA; rA; rA; rG; rU; rG; rG; rC; |
| | | rA; rC; rC; rG; rA; rG; rU; rC; rG; rG; rU; rG; rC; rUs; mUs; mUs; mU |
| GD-28435 | 72 | mAs; mUs; mGs; rG; rU; rU; rA; rA; rU; rC; rG; rG; rU; rC; rC; rC; rC; rC; rG; rC; |
| | | rG; rU; rU; rU; rU; rA; rG; rA; rG; rC; rU; rA; rG; rA; rA; rA; rU; rA; rG; rC; rA; |
| | | rA; rG; rU; rU; rA; rA; rA; rA; rU; rA; rA; rG; rG; rC; rU; rA; rG; rU; rC; rC; rG; |
| | | rU; rU; rA; rU; rC; rA; rA; rC; rU; rU; rG; rA; rA; rA; rA; rA; rG; rU; rG; rG; rC; |
| | | rA; rC; rC; rG; rA; rG; rU; rC; rG; rG; rU; rG; rC; rUs; mUs; mUs; mU |
| GD-28436 | 73 | mGs; mUs; mCs; rC; rU; rC; rU; rG; rG; rG; rA; rA; rG; rA; rU; rC; rU; rG; rC; rU; |
| | | rG; rU; rU; rU; rU; rA; rG; rA; rG; rC; rU; rA; rG; rA; rA; rA; rU; rA; rG; rC; rA; |
| | | rA; rG; rU; rU; rA; rA; rA; rA; rU; rA; rA; rG; rG; rC; rU; rA; rG; rU; rC; rC; rG; |
| | | rU; rU; rA; rU; rC; rA; rA; rC; rU; rU; rG; rA; rA; rA; rA; rA; rG; rU; rG; rG; rC; |
| | | rA; rC; rC; rG; rA; rG; rU; rC; rG; rG; rU; rG; rC; rUs; mUs; mUs; mU |
| GD-28437 | 74 | mGs; mGs; mUs; rU; rU; rG; rA; rA; rA; rG; rG; rA; rA; rG; rG; rC; rA; rG; rA; rG; |
| | | rG; rU; rU; rU; rU; rA; rG; rA; rG; rC; rU; rA; rG; rA; rA; rA; rU; rA; rG; rC; rA; |
| | | rA; rG; rU; rU; rA; rA; rA; rA; rU; rA; rA; rG; rG; rC; rU; rA; rG; rU; rC; rC; rG; |
| | | rU; rU; rA; rU; rC; rA; rA; rC; rU; rU; rG; rA; rA; rA; rA; rA; rG; rU; rG; rG; rC; |
| | | rA; rC; rC; rG; rA; rG; rU; rC; rG; rG; rU; rG; rC; rUs; mUs; mUs; mU |
| GD-28438 | 75 | mAs; mCs; mCs; rC; rU; rG; rG; rG; rC; rG; rC; rC; rC; rA; rC; rC; rC; rC; rG; rA; |
| | | rG; rU; rU; rU; rU; rA; rG; rA; rG; rC; rU; rA; rG; rA; rA; rA; rU; rA; rG; rC; rA; |
| | | rA; rG; rU; rU; rA; rA; rA; rA; rU; rA; rA; rG; rG; rC; rU; rA; rG; rU; rC; rC; rG; |
| | | rU; rU; rA; rU; rC; rA; rA; rC; rU; rU; rG; rA; rA; rA; rA; rA; rG; rU; rG; rG; rC; |
| | | rA; rC; rC; rG; rA; rG; rU; rC; rG; rG; rU; rG; rC; rUs; mUs; mUs; mU |
| GD-28439 | 76 | mUs; mUs; mCs; rU; rC; rC; rC; rU; rG; rC; rC; rU; rC; rC; rA; rC; rG; rC; rC; rG; |
| | | rG; rU; rU; rU; rU; rA; rG; rA; rG; rC; rU; rA; rG; rA; rA; rA; rU; rA; rG; rC; rA; |
| | | rA; rG; rU; rU; rA; rA; rA; rA; rU; rA; rA; rG; rG; rC; rU; rA; rG; rU; rC; rC; rG; |
| | | rU; rU; rA; rU; rC; rA; rA; rC; rU; rU; rG; rA; rA; rA; rA; rA; rG; rU; rG; rG; rC; |
| | | rA; rC; rC; rG; rA; rG; rU; rC; rG; rG; rU; rG; rC; rUs; mUs; mUs; mU |

TABLE 3
Site-Specific HNF4α Promoter 2 Targeting Moieties (sgRNA) - The first 20
nucleotides in each moiety below comprise the targeting portion of the moiety.

| | SEQ | |
|---|---|---|
| | ID | |
| Identifier | NO. | Modified Nucleotide Sequence 5′ to 3′ |
| GD28427 | 77 | mAs; mUs; mGs; rC; rC; rC; rC; rC; rA; rG; rC; rU; rC; rU; rC; rC; rG; rG; rC; rU; |
| | | rG; rU; rU; rU; rU; rA; rG; rA; rG; rC; rU; rA; rG; rA; rA; rA; rU; rA; rG; rC; rA; |
| | | rA; rG; rU; rU; rA; rA; rA; rA; rU; rA; rA; rG; rG; rC; rU; rA; rG; rU; rC; rC; rG; |
| | | rU; rU; rA; rU; rC; rA; rA; rC; rU; rU; rG; rA; rA; rA; rA; rA; rG; rU; rG; rG; rC; |
| | | rA; rC; rC; rG; rA; rG; rU; rC; rG; rG; rU; rG; rC; rUs; mUs; mUs; mU |
| GD28428 | 78 | mCs; mAs; mGs; rC; rG; rU; rG; rA; rA; rC; rG; rC; rG; rC; rC; rC; rC; rU; rC; rG; |
| | | rG; rU; rU; rU; rU; rA; rG; rA; rG; rC; rU; rA; rG; rA; rA; rA; rU; rA; rG; rC; rA; |
| | | rA; rG; rU; rU; rA; rA; rA; rA; rU; rA; rA; rG; rG; rC; rU; rA; rG; rU; rC; rC; rG; |
| | | rU; rU; rA; rU; rC; rA; rA; rC; rU; rU; rG; rA; rA; rA; rA; rA; rG; rU; rG; rG; rC; |
| | | rA; rC; rC; rG; rA; rG; rU; rC; rG; rG; rU; rG; rC; rUs; mUs; mUs; mU |
| GD28429 | 79 | mCs; mUs; mUs; rA; rC; rG; rG; rU; rA; rA; rG; rU; rG; rG; rG; rG; rC; rU; rG; rG; |
| | | rG; rU; rU; rU; rU; rA; rG; rA; rG; rC; rU; rA; rG; rA; rA; rA; rU; rA; rG; rC; rA; |
| | | rA; rG; rU; rU; rA; rA; rA; rA; rU; rA; rA; rG; rG; rC; rU; rA; rG; rU; rC; rC; rG; |
| | | rU; rU; rA; rU; rC; rA; rA; rC; rU; rU; rG; rA; rA; rA; rA; rA; rG; rU; rG; rG; rC; |
| | | rA; rC; rC; rG; rA; rG; rU; rC; rG; rG; rU; rG; rC; rUs; mUs; mUs; mU |
| GD28430 | 80 | mCs; mCs; mCs; rG; rU; rA; rA; rG; rA; rA; rA; rC; rA; rC; rA; rC; rG; rG; rG; rG; rG; |
| | | rU; rU; rU; rU; rA; rG; rA; rG; rC; rU; rA; rG; rA; rA; rA; rU; rA; rG; rC; rA; rA; |
| | | rG; rU; rU; rA; rA; rA; rA; rU; rA; rA; rG; rG; rC; rU; rA; rG; rU; rC; rC; rG; rU; |
| | | rU; rA; rU; rC; rA; rA; rC; rU; rU; rG; rA; rA; rA; rA; rA; rG; rU; rG; rG; rC; rA; |
| | | rC; rC; rG; rA; rG; rU; rC; rG; rG; rU; rG; rC; rUs; mUs; mUs; mU |
| GD28431 | 68 | mAs; mUs; mUs; rG; rA; rA; rU; rU; rA; rG; rG; rG; rG; rA; |
| | | rU; rC; rU; rC; rG; rG; rG; rU; rU; rU; rU; rA; rG; rA; rG; rC; rU; rA; rG; rA; rA; |
| | | rA; rU; rA; rG; rC; rA; rA; rG; rU; rU; rA; rA; rA; rA; rU; rA; rA; rG; rG; rC; rU; |
| | | rA; rG; rU; rC; rC; rG; rU; rU; rA; rU; rC; rA; rA; rC; rU; rU; rG; rA; rA; rA; rA; |
| | | rA; rG; rU; rG; rG; rC; rA; rC; rC; rG; rA; rG; rU; rC; rG; rG; rU; rG; rC; rUs; |
| | | mUs; mUs; mU |

TABLE 4
Unmodified Nucleotide Sequences of the Targeting Portion of the Site-Specific HNF4α
Targeting Moieties in Tables 2 and 3.

| | SEQ | | SEQ | | |
|---|---|---|---|---|---|
| | ID | Unmodified Nucleotide Sequence | ID | Reverse Complement Nucleotide | Chromosomal |
| Identifier | NO. | 5′ to 3′ | NO. | Sequence 5′ to 3′ | Coordinates |
| GD-28427 | 329 | ATGCCCCCAGCTCTCCGGCT | 335 | AGCCGGAGAGCTGGGGGCAT | chr20: 42984411- |
| | | | | | 42984433 |
| GD-28428 | 330 | CAGCGTGAACGCGCCCCTCG | 336 | CGAGGGGCGCGTTCACGCTG | chr20: 42984450- |
| | | | | | 42984472 |
| GD-28429 | 331 | CTTACGGTAAGTGGGGCTGG | 337 | CCAGCCCCACTTACCGTAAG | chr20: 42984488- |
| | | | | | 42984510 |
| GD-28430 | 332 | CCCGTAAGAAACACACGGGG | 338 | CCCCGTGTGTTTCTTACGGG | chr20: 42984560- |
| | | | | | 42984582 |
| GD-28431 | 333 | ATTGAATTAGGGGATCTCGG | 339 | CCGAGATCCCCTAATTCAAT | chr20: 43029535- |
| | | | | | 43029557 |
| GD-28432 | 334 | GACTTGGGGTGACAATGGCT | 340 | AGCCATTGTCACCCCAAGTC | chr20: 43029596- |
| | | | | | 43029618 |
| GD-28433 | 81 | AACTGAACATCGGTGAGTTA | 82 | TAACTCACCGATGTTCAGTT | chr20: 43029685- |
| | | | | | 43029707 |
| GD-28434 | 83 | TGGTTTCTGGCTGACACCCG | 84 | CGGGTGTCAGCCAGAAACCA | chr20: 43029729- |
| | | | | | 43029751 |
| GD-28435 | 85 | ATGGTTAATCGGTCCCCCGC | 86 | GCGGGGGACCGATTAACCAT | chr20: 43029792- |
| | | | | | 43029814 |
| GD-28436 | 87 | GTCCTCTGGGAAGATCTGCT | 88 | AGCAGATCTTCCCAGAGGAC | chr20: 43029873- |
| | | | | | 43029895 |
| GD-28437 | 89 | GGTTTGAAAGGAAGGCAGAG | 90 | CTCTGCCTTCCTTTCAAACC | chr20: 43029896- |
| | | | | | 43029918 |
| GD-28438 | 91 | ACCCTGGGCGCCCACCCCGA | 92 | TCGGGGTGGGCGCCCAGGGT | chr20: 43029957- |
| | | | | | 43029979 |
| GD-28439 | 93 | TTCTCCCTGCCTCCACGCCG | 94 | CGGCGTGGAGGCAGGGAGAA | chr20: 43029991- |
| | | | | | 43030013 |

TABLE 5
Abbreviations of nucleotide monomers used in nucleic
acid sequence representation. It will be understood
that these monomers, when present in an oligonucleotide,
are mutually linked by 5′-3′-phosphodiester bonds.

| Abbreviation | Nucleotide(s) |
|---|---|
| A | Adenosine-3′-phosphate |
| As | adenosine-3′-phosphorothioate |
| C | cytidine-3′-phosphate |
| Cs | cytidine-3′-phosphorothioate |
| G | guanosine-3′-phosphate |
| Gs | guanosine-3′-phosphorothioate |
| U | Uridine-3′-phosphate |
| Us | uridine-3′-phosphorothioate |
| N | any nucleotide, modified or unmodified |
| mA | 2′-O-methyladenosine-3′-phosphate |
| mAs | 2′-O-methyladenosine-3′-phosphorothioate |
| mC | 2′-O-methylcytidine-3′-phosphate |
| mCs | 2′-O-methylcytidine-3′-phosphorothioate |
| mG | 2′-O-methylguanosine-3′-phosphate |
| mGs | 2′-O-methylguanosine-3′-phosphorothioate |
| mU | 2′-O-methyluridine-3′-phosphate |
| mUs | 2′-O-methyluridine-3′-phosphorothioate |
| s | phosphorothioate linkage |
| r | ribonucleotide |

### Example 3. Design of Zinc Finger DNA Binding Domain and TALE Fusion Protein

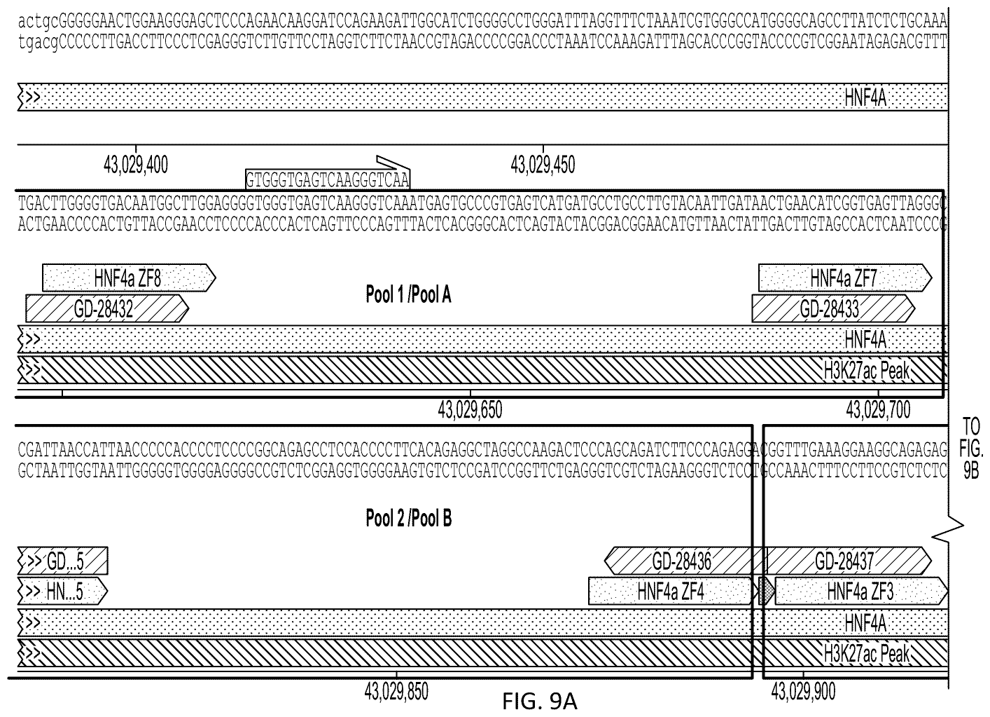

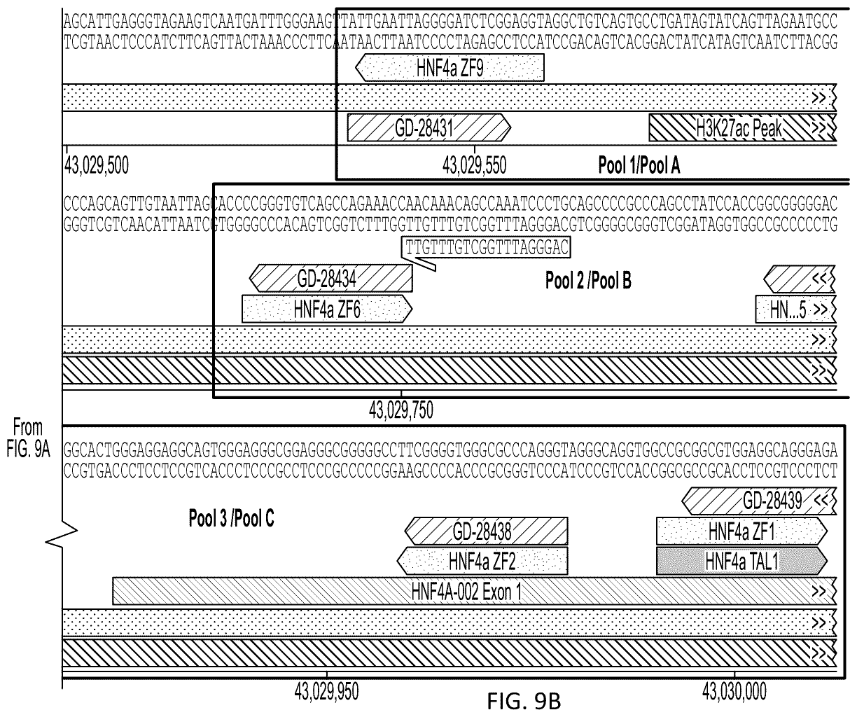

As described in Examples 1 and 2, the sites of effective activation of HNF4α gene expression were identified using dCas9 fusion proteins and guides directed to specific nucleotide regions 5′ of promoter 1 of the HNF4α gene. Based on these data, Zinc Finger DNA binding domain polypeptides (ZF) and TALE polypeptides were designed to target the same or similar sequence regions 5′ of promoter 1 of the HNF4α gene. FIGS. 9A and 9B depict the target areas for design of the exemplary ZF proteins and TALE proteins of the inventions.

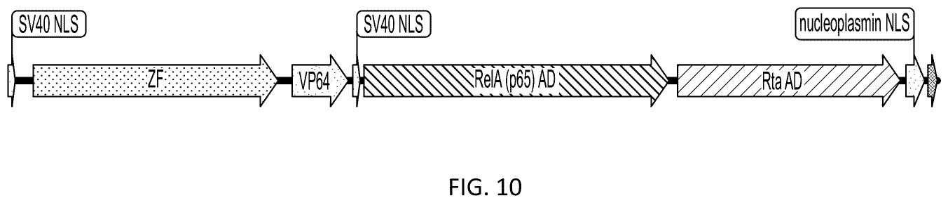

FIG. 10 depicts the structure of the exemplary site-specific HNF4α disrupting agent, comprising a site-specific HNF4α targeting moiety which targets an HNF4α expression control region, e.g., a zing finger (ZF) or a TALE, and an effector comprising, e.g., VPR (e.g., ZF-VPR or TALE-VPR fusion proteins) of the invention. As shown in FIG. 10, an exemplary ZF-VPR protein may include one or more nuclear localization signals (NLS), such as SV40 NLS or nucleoplasmin NLS. In some embodiments, the NLSs are located at the N-terminus, between the VP64 and RelA (p65) activation domain (VPR), and at the C-terminus.

In some embodiments, the mRNAs encoding the ZF fusion protein or the TAL fusion proteins, may contain a “natural cap” structure at the 5′-terminus, e.g., the mRNAs may be cap0 (no methyl), cap1 (methyl on first ribose), cap2 (methyl on second ribose).

Downstream of the cap is a 5′ untranslated region (UTR), a sequence which is designed to promote high levels of protein translation. Downstream of the 5′ UTR is the coding sequence, 3′ UTR and the polyA tail.

An exemplary nucleotide sequence of a 5′ UTR for use in the constructs is

(SEQ ID NO.: 341)
AGGAAAUAAGAGAGAAAAGAAGAGUAAGAAGAAAUAUAAGAGCCACC.

An exemplary nucleotide sequence of a 3′ UTR for use in the constructs is

(SEQ ID NO.: 342)
CUCUCCCUUGCACCUGUACCUCUUGGUCUUUGAAUAAAGCCUGAGUAGGA
AGUCUAG

An exemplary nucleotide sequence of a poly-A tail for use in the constructs is

(SEQ ID NO.: 343)
AAAAAAAAAAAAAAAAAAAAAAAAAAAAAAAAAAAAAAAAAAAAAAAAAA
AAAAAAAAAAAAAAAAAAAAAAAAAAAA

The mRNAs encoding the ZF fusion proteins or the TALE fusion proteins may be complexed with a lipid nanoparticle (e.g., MC3). The ZF domains of the fusion proteins are targeted to P1 of HNF4α, which controls the transcription of the isoforms of HNF4α expressed primarily in the liver.

Tables 6A and 6B below provide the amino acid sequences of various exemplary fusion protein constructs, the corresponding encoding mRNA sequences, the target genomic sequences thereof, and the amino acid sequences of the DNA binding domains for use in the disrupting agents of the constructs.

TABLE 6A

| | | | | | Column 6 | Column 7 |
|---|---|---|---|---|---|---|
| | | | Column 4 | Column 5 | Nucleotide | Genomic Coordinates of |
| | | | Sequence of | Amino Acid | Sequence | the Target Site in the |
| Column 1 | Column 2 | Column 3 | Target | Sequence of the | of the | HNF4α Expression |
| Exemplary HNF4α | Amino Acid | Nucleotide | Site in HNF4α | DNA Binding | DNA Binding | Control Region |
| Disrupting Agents | Sequence of the | Sequence of | Expression Control | Domain of the | Domain of the | (Genome Reference |
| Comprising a Zinc | Disrupting | the Disrupting | Region Targeted by | Disrupting | Disrupting | Consortium |
| Finger DNA Binding | Agent in | Agents in | the Disrupting | Agent in | Agent in | Human Build |
| Domain and an Effector | Column 1 | Column 1 | Agents in Column 1 | Column 1 | Column 1 | 37 (GRCh37): |
| Name | (SEQ ID NO:) | (SEQ ID NO). | (SEQ ID NO.) | (SEQ ID NO.) | (SEQ ID NO.) | Chromosome 20) |
| ZF1-VPR | 100 | 101 | 102 | 103 | 211 | GrCh37: chr20:43029991- |
| | | | | | | 43030011 |
| ZF2-VPR | 104 | 105 | 106 | 107 | 212 | GrCh37: chr20:43029959- |
| | | | | | | 43029979 |
| ZF3-VPR | 108 | 109 | 110 | 111 | 213 | GrCh37: chr20:43029897- |
| | | | | | | 43029917 |
| ZF4-VPR | 112 | 113 | 114 | 115 | 214 | GrCh37: chr20:43029874- |
| | | | | | | 43029894 |
| ZF5-VPR | 116 | 117 | 118 | 119 | 215 | GRCh37: chr20:43029794- |
| | | | | | | 43029814 |
| ZF6-VPR | 120 | 121 | 122 | 123 | 216 | CRCh37: chr20:43029731- |
| | | | | | | 43029751 |
| ZF7-VPR | 124 | 125 | 126 | 127 | 217 | GRCh37: chr20:43029686- |
| | | | | | | 43029706 |
| ZF8-VPR | 128 | 129 | 130 | 131 | 218 | GrCh37: chr20:43029598- |
| | | | | | | 43029618 |
| ZF9-VPR | 132 | 133 | 134 | 135 | 219 | GrCh37: chr20:43029536- |
| | | | | | | 43029558 |
| ZF10-VPR | 136 | 137 | 138 | 139 | 220 | GrCh37: chr20:43029767- |
| | | | | | | 43029787 |
| ZF11-VPR | 140 | 141 | 142 | 143 | 221 | GrCh37: chr20:43029820- |
| | | | | | | 43029840 |
| ZF12-VPR | 144 | 145 | 146 | 147 | 222 | GrCh37: chr20:43029855- |
| | | | | | | 43029875 |
| ZF13-VPR | 148 | 149 | 150 | 151 | 223 | GrCh37: chr20:43029766- |
| | | | | | | 43029786 |
| ZF14-VPR | 152 | 153 | 154 | 155 | 224 | GRCh37: chr20:43029810- |
| | | | | | | 43029830 |
| ZF15-VPR | 156 | 157 | 158 | 159 | 225 | GRCh37: chr20:43029832- |
| | | | | | | 43029852 |
| ZF5-VPR ATUM Opt_1 | 116 | 160 | 118 | 119 | 226 | GRCh37: chr20:43029794- |
| (ZF5.1-VPR) | | | | | | 43029814 |
| ZF5-VPR ATUM Opt_2 | 116 | 161 | 118 | 119 | 227 | GRCh37: chr20:43029794- |
| (ZF5.2-VPR) | | | | | | 43029814 |
| ZF5-VPR ATUM Opt_3 | 116 | 162 | 118 | 119 | 228 | GRCh37: chr20:43029794- |
| (ZF5.3-VPR) | | | | | | 43029814 |
| ZF5-VPR ATUM Opt_4 | 116 | 163 | 118 | 119 | 229 | GRCh37: chr20:43029794- |
| (ZF5.4-VPR) | | | | | | 43029814 |
| ZF5-VPR ATUM Opt_5 | 116 | 164 | 118 | 119 | 230 | GRCh37: chr20:43029794- |
| (ZF5.5-VPR) | | | | | | 43029814 |
| ZF5-VPR ATUM Opt_6 | 116 | 165 | 118 | 119 | 231 | GRCh37: chr20:43029794- |
| (ZF5.6-VPR) | | | | | | 43029814 |
| ZF5-P300 | 166 | 167 | 118 | 119 | 232 | GRCh37: chr20:43029794- |
| | | | | | | 43029814 |
| ZF5-VPR + ZF7-VPR | 116 + 124 | 117 + 125 | 118 + 126 | 119 + 127 | 215 + 217 | GRCh37: chr20:43029794- |
| | | | | | | 43029814 |
| | | | | | | GRCh37: chr20:43029686- |
| | | | | | | 43029706 |
| ZF5-VPR ATUMOpt_3 + | 116 + 124 | 162 + 125 | 118 + 126 | 119 + 127 | 228 + 217 | GRCh37: chr20:43029794- |
| ZF7-VPR | | | | | | 43029814 |
| | | | | | | GRCh37: chr20:43029686- |
| | | | | | | 43029706 |
| ZF7-P300 | 168 | 169 | 126 | 127 | 233 | GRCh37: chr20:43029686- |
| | | | | | | 43029706 |
| ZF5.3-VPR3 | 170 | 171 | 118 | 119 | 234 | GRCh37: chr20:43029794- |
| | | | | | | 43029814 |
| ZF5-no effector | 174 | 175 | 118 | 119 | 235 | GRCh37: chr20:43029794- |
| | | | | | | 43029814 |
| ZF5.3-VPR-tPT2a-ZF7- | 176 | 177 | 118 + 126 | 119 + 127 | 236 + 237 | GRCh37: chr20:43029794- |
| VPR | | | | | | 43029814 |
| | | | | | | GRCh37: chr20:43029686- |
| | | | | | | 43029706 |
| ZF7-VPR-tPT2a-ZF5.3- | 178 | 179 | 126 + 118 | 127 + 119 | 238 + 239 | GRCh37: chr20:43029686- |
| VPR | | | | | | 43029706 |
| | | | | | | GRCh37: chr20:43029794- |
| | | | | | | 43029814 |
| ZF5.3-VPR-tPT2a-ZF7- | 180 | 181 | 118 + 126 | 119 + 127 | 240 + 241 | GRCh37: chr20:43029794- |
| p300 | | | | | | 43029814 |
| | | | | | | GRCh37: chr20:43029686- |
| | | | | | | 43029706 |
| ZF7-p300-tPT2a-ZF5.3- | 182 | 183 | 126 + 118 | 127 + 119 | 242 + 243 | GRCh37: chr20:43029686- |
| VPR | | | | | | 43029706 |
| | | | | | | GRCh37: chr20:43029794- |
| | | | | | | 43029814 |
| TAL1-VPR | 184 | 185 | 102 | 186 | 244 | GrCh37: chr20:43029991- |
| | | | | | | 43030011 |
| TAL2-VPR | 187 | 188 | 106 | 189 | 245 | GrCh37: chr20:43029959- |
| | | | | | | 43029979 |
| TAL3-VPR | 190 | 191 | 110 | 192 | 246 | GrCh37: chr20:43029897- |
| | | | | | | 43029917 |
| TAL4-VPR | 193 | 194 | 114 | 195 | 247 | GrCh37: chr20:43029874- |
| | | | | | | 43029894 |
| TAL5-VPR | 196 | 197 | 118 | 198 | 248 | GRCh37: chr20:43029794- |
| | | | | | | 43029814 |
| TAL6-VPR | 199 | 200 | 122 | 201 | 249 | CRCh37: chr20:43029731- |
| | | | | | | 43029751 |
| TAL7-VPR | 202 | 203 | 126 | 204 | 250 | GRCh37: chr20:43029686- |
| | | | | | | 43029706 |
| TAL8-VPR | 205 | 206 | 130 | 207 | 251 | GrCh37: chr20:43029598- |
| | | | | | | 43029618 |
| TAL9-VPR | 208 | 209 | 134 | 210 | 252 | GrCh37: chr20:43029536- |
| | | | | | | 43029558 |

TABLE 6B

| Column 1 | | | Column 4 |
|---|---|---|---|
| Exemplary Effector | Column 2 | Column 3 | Amino acid Sequence of |
| Fusion Protein of | Amino acid sequence of | Nucleotide sequence of | DNA Binding Domain |
| Exemplary HNF4α | the Effector Fusion | the Effector Fusion | of the Effector Fusion |
| Disrupting Agents | Protein in Column 1 | Protein in Column 1 | Protein in Column 1 |
| Name | (SEQ ID NO:) | (SEQ ID NO:) | (SEQ ID NO:) |
| dCas9-VPR | 95 | 96 | 97 |
| dCas9-P300 | 98 | 99 | 97 |
| dCas9-VPR3 | 172 | 173 | 97 |

### Example 4. Modulation of HNF4α Expression by ZF-Fusion Proteins

In order to test a single mRNA encoding a site-specific HNF4α disrupting agent, comprising a site-specific HNF4α targeting moiety which targets an HNF4α expression control region, e.g., a zing finger (ZF), and an effector comprising, e.g., VPR, (ZF-VPR fusion proteins), rather than dCas9-effector fusions with pooled sgRNAs, Zinc Finger DNA Binding Domain (DBD)-VPR (ZF-VPR) fusion proteins were designed as described above. Such fusion proteins which bind sites similar to or identical to the P1 targeting guide RNAs described in Examples 1 and 2 were evaluated for their ability to effect expression of HNF4α in vitro.

Several mRNAs encoding fusion proteins comprising a ZF DBD and a VPR domain were constructed (see Table 6A). Individual and pooled fusion protein encoding mRNAs were delivered to LX-2 cells as MC3 LNP formulations as described above. Expression of HNF4α was measured by qPCR as described above. Untreated HepG2 and dCas9-VPR-Pool 1 were included as positive controls.

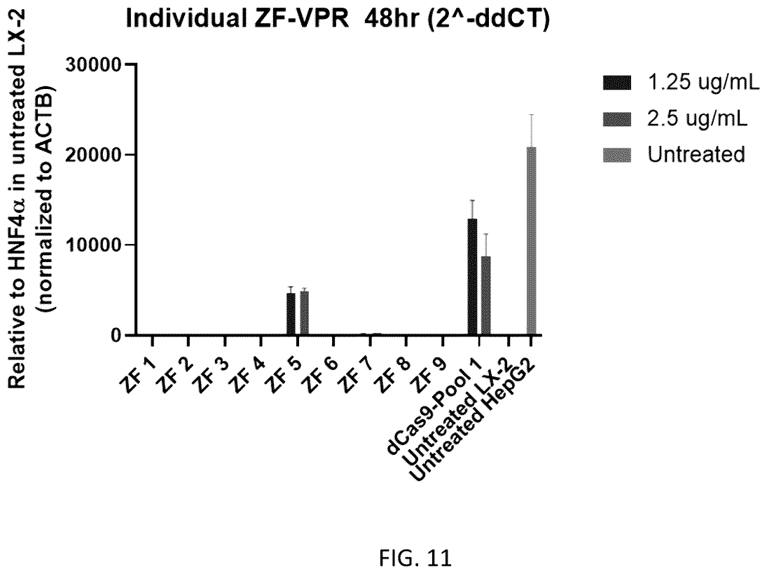

As shown in FIG. 11, it was found that ZF005-VPR (also referred to as ZF5-VPR) showed robust upregulation of HNF4α. ZF007-VPR (also referred to as ZF7-VPR) also showed a quantifiable upregulation.

### Example 5. Modulation of HNF4α Expression by TALE-fusion proteins

In a further effort to use a single mRNA coding a site-specific HNF4α disrupting agent, comprising a site-specific HNF4α targeting moiety which targets an HNF4α expression control region, e.g., a Talen based DNA Binding Domain (DBD), and an effector comprising, e.g., VPR, (TAL-VPR fusion proteins), were designed. Such fusion proteins which bind sites similar to or identical to the P1 targeting guide RNAs described in Examples 1 and 2 were evaluated for their ability to effect expression of HNF4α in vitro.

Several mRNAs encoding fusion proteins of a TALEN DBD with a VPR domain were constructed (see Table 6A). Individual and pooled fusion protein encoding mRNAs were delivered to LX-2 cells as MC3 LNP formulations as described above. Expression of HNF4α was measured by qPCR as described above. Untreated HepG2 and dCas9-VPR-Pool 1 were included a positive controls. ZF5-VPR and ZF7-VPR were also included in the study as comparators.

Table 7 below shows the experiment design for the pooled TAL-VPR experiments.

TABLE 7

| | | |
|---|---|---|
| TAL1-VPR | Pool X | Pools are made by mixing 1:1:1 |
| TAL2-VPR | | of each TAL fusion protein. |
| TAL3-VPR | | |
| TAL4-VPR | Pool Y | |
| TAL5-VPR | | |
| TAL6-VPR | | |
| TAL7-VPR | Pool Z | |
| TAL8-VPR | | |
| Unrelated domain-VPR | | |

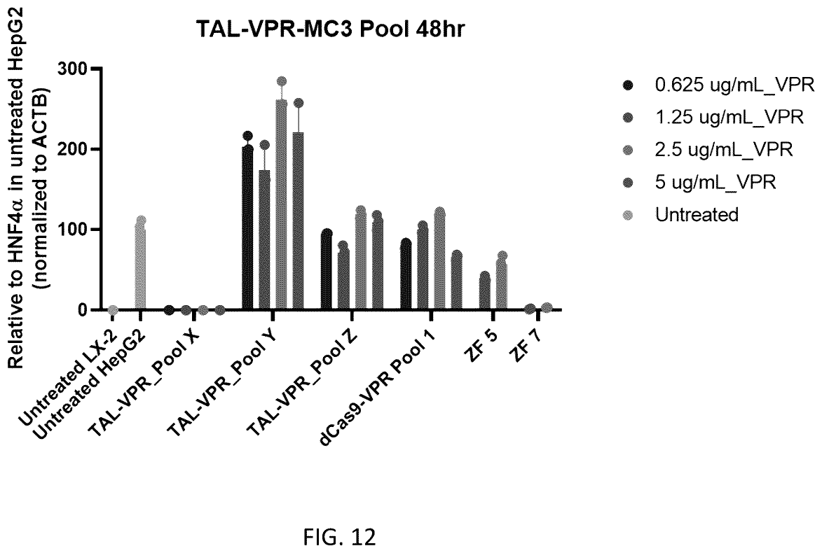

As shown in FIG. 12, it was found that pooled TALE based-VPR fusion proteins showed strong up-regulation of HNF4α expression Individually, each TAL-VPR fusion protein did not have significant activity to up-regulate the expression of HNF4α (data not shown). This Figure also demonstrates that the results described above for ZF5-VPR and Z7-VPR were reproducible.

### Example 6. Modulation of HNF4α Expression with Codon Optimized ZF5-VPR and Synergy of the Combination of ZF5-VPR and ZF7-VPR in LX-2 Cells

In this example, activation of HNF4α in LX-2 cells with codon optimized ZF5-VPR fusion proteins was evaluated. The objective was to analyze whether codon optimization of ZF5-VPR would improve activity of the ZF5-VPR fusion proteins at the HNF4α promoter. Another objective was to analyze whether the combination of ZF5-VPR and ZF7-VPR fusion proteins was synergistic in activating HNF4α expression.

The sequence for ZF5-VPR fusion proteins were sent to ATUM for codon optimization, a process that uses al algorithm to re-design ideal codon frequency and context to optimize translational efficiency. ATUM returned 6 new variants which all encode the same protein, each with different RNA sequences. (see Table 6A).

mRNAs for all five ATUM codon optimized ZF5-VPR, unaltered ZF5-VPR and ZF7-VPR were transfected into LX-2 cells as MC3 LNPs. Expression of HNF4α was measured by qPCR 48 hours after transfection.

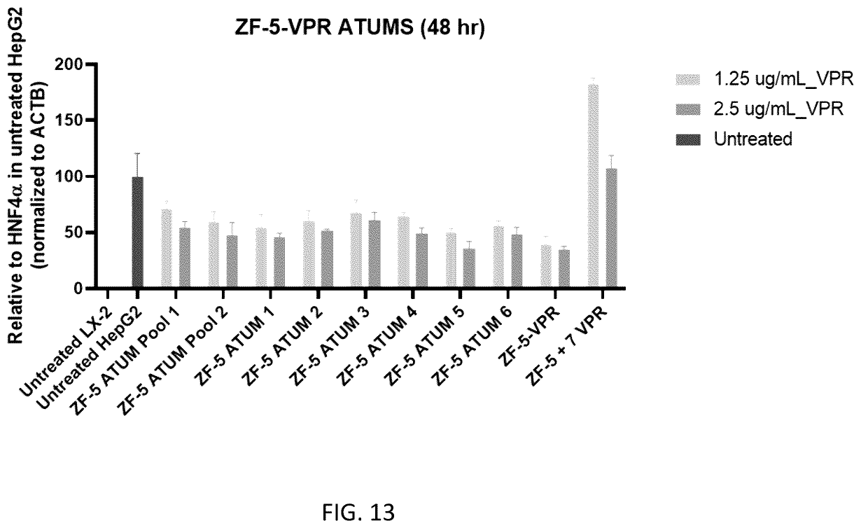

As shown in FIG. 13, ZF-5-VPR ATUM codon optimized variant 3 (ZF5.3-VPR) showed stronger upregulation of HNF4α expression as compared to the other codon-optimized variants. Co-transfecting ZF5-VPR and ZF7-VPR led to a supraphysiological (>200%) increase in expression of HNF4α, demonstrating synergy of the combination of these two fusion proteins. (In FIG. 13, Pool 1 is dCas9-VPR+GD-28431+GD-28432+GD-28433; Pool 2 is dCas9-VPR+GD-28434+GD-28435+GD-28436).

### Example 7. Durability of Modulation of HNF4α Expression in K562 Cells

This example identified the effectors (VPR and/or P300) that was able to induce the longest lasting up-regulation of HNF4α expression. The example also measures how long the up-regulation of HNF4α in K562 could last when treated with various ZF-effector fusion proteins and various combinations of ZF-effector fusion proteins.

Various individual mRNAs encoding ZF-effector fusion proteins and combinations of the mRNAs were transfected into K562 cells and allowed to grow for 10 days in culture.

The following individual fusion proteins and combinations were tested in K562 cells, transfected with MC3 LNPs: ZF5-VPR, ZF5-P300, a combination of ZF5-VPR and ZF5-P300, or a combination of ZF5-VPR and ZF7-VPR (see Table 6A).

Transfections and quantifications were performed as described above. Briefly, untreated K562 cells were included as an assay control. K562 cells were treated with individual and different combination of effectors (2.5 μg/mL) in triplicate. Data points were collected over a period of 10 days. qPCR readout was used to measure mRNA expression. K562 cells were seeded at 100 k/well in triplicates: Time points were collected every 2 days for a total of 10 days. Three wells of untreated K562 were included as a control. RT qPCR for HNF4α was performed to measure the expression of HNF4α at each time point for similarly treated LX-2 cells.

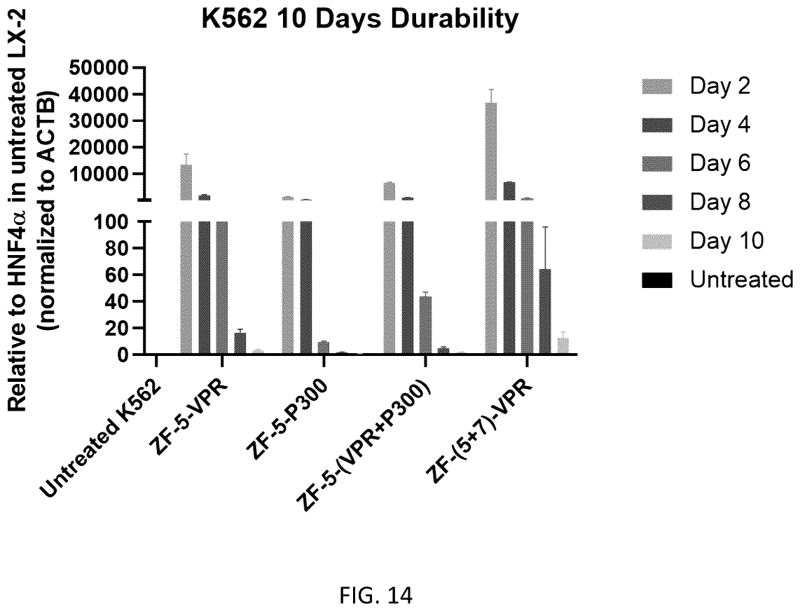

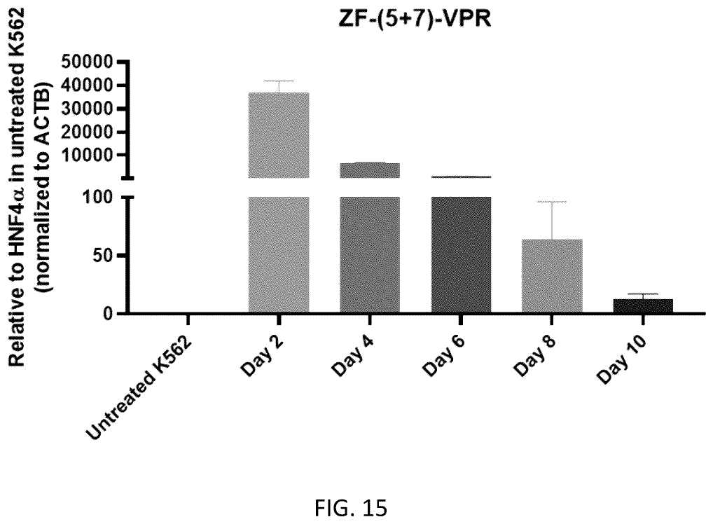

As shown in FIGS. 14 and 15, HNF4α expression upregulation was observed in K562 cell until day 6 when any of the single fusion proteins or combinations of the fusion proteins were transfected. Co-transfection with mRNAs encoding ZF5-VPR and ZF7-VPR led to the highest and most durable increase of HNF4α expression in cultured cells, with detectable expression out to 10 days.

In another experiment, the durability of up-regulation of HNF4α expression in K562 when treated with ZF-5-VPR, ZF-5-P300, a combination of ZF5-VPR-ATUM 3 and ZF7-VPR (also referred to as ZF (5.3+7) or a combination of ZF5-PR and ZF7-VPR (also referred to as ZF-(5+7)-VPR) was determined.

K562 cells were treated with individual and different combination of effectors (2.5 ug/mL) in triplicate. Time points were collected over a period of 10 days. qPCR readout was used to measure mRNA expression. K562 cells were seeded at 100 k/well in triplicates: Time points were collected every 2 days for a total of 10 days. Three wells of untreated K562 were included as a control. RT qPCR was performed to measure the expression of HNF4α at each time point. Fusion protein were formulated in MC3 LNPs.

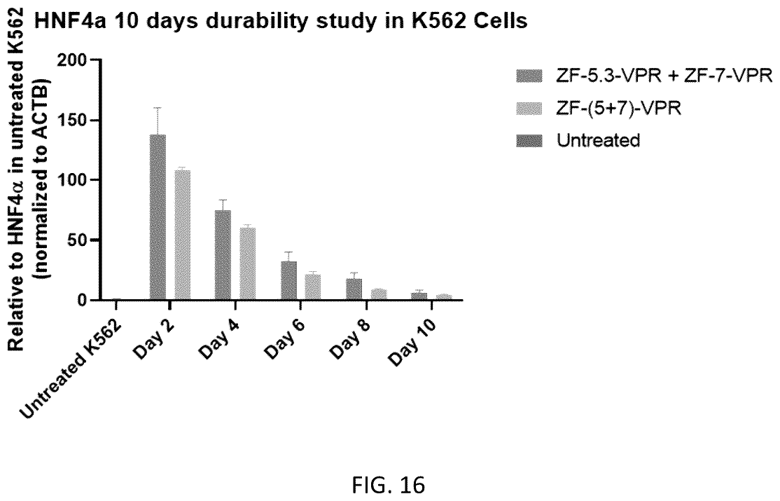

As shown in FIG. 16, HNF4α upregulation was observed in K562 cell until day 6 using both combinations of ZF5-VPR and ZF7-VPR and ZF5.3-VPR and ZF7-VPR. Both combinations of ZF5-VPR and ZF7-VPR and ZF5.3-VPR and ZF7-VPR showed slight upregulation at day 8 and 10.

### Example 8. Modulation of Biomarker Genes Expression Following Activation of HNF4α in LX-2 Cells

The first experiment of this example demonstrates the change in expression level of downstream biomarker genes following activation of HNF4α in LX-2 Cells. The objective of this experiment was to demonstrate that the HNF4α induced by dCas9-VPR in LX-2 cells is an active transcription factor by examining the effect of upregulating HNF4α expression on the expression of two downstream gene Cola1 and αSMA. Collagen 1a1 (Col1a1), and alpha-Smooth Muscle Actin (αSMA) are 2 proteins highly expressed in damaged liver cells and are key drivers of fibrosis in end-stage liver disease. Both of these genes are negatively-regulated by HNF4α.

LX-2 cells, which highly express Col1a1 and αSMA, were transfected with dCas-VPR-Pool 1 as described above, followed by qPCR measurement of HNF4A, Col1a1, and αSMA.

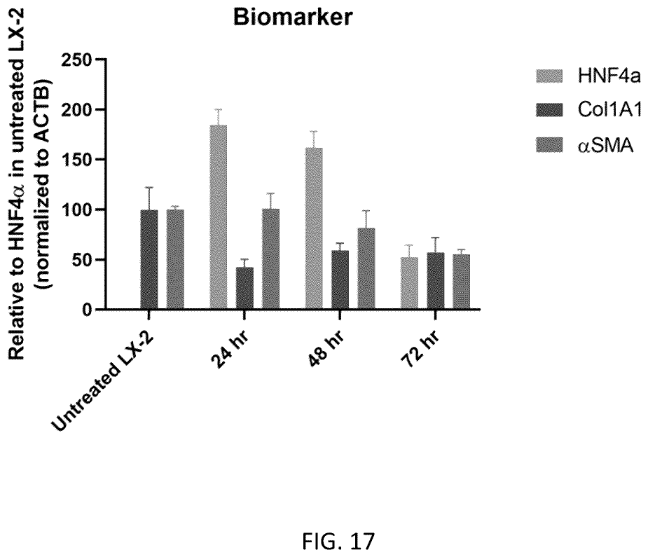

As shown in FIG. 17, upregulation of HNF4α significantly down-regulated expression of Col1a1 and αSMA.

The second experiment of this example demonstrates that the ZF-VPR fusion proteins also down-regulate expression of Col1a1 and αSMA, biomarkers of fibrotic liver disease.

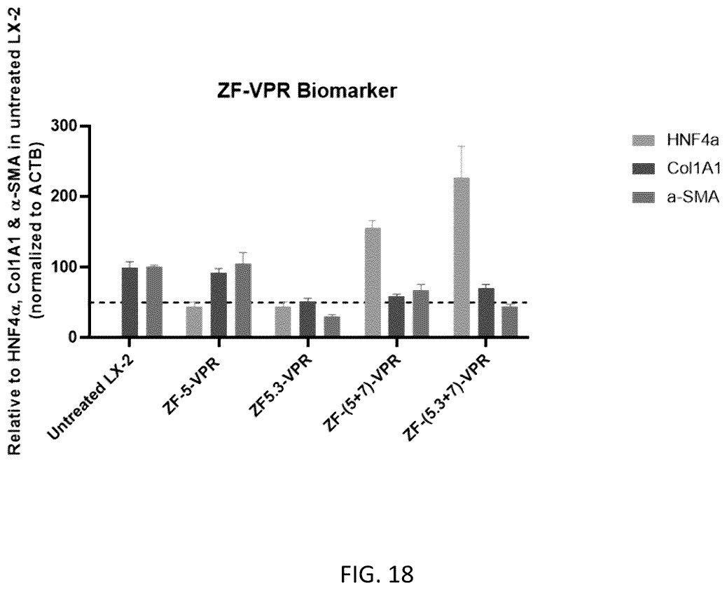

As shown in FIG. 18, biomarkers of fibrotic liver disease. Cola1 and αSMA, were down-regulated in LX-2 cells following up-regulation of HNF4α with ZF5.3-VPR, a combination of ZF5-VPR and ZF7-VPR or a combination of ZF5.3-VPR and ZF7-VPR.

### Example 9. Screening of Additional ZF-VPR Fusion Proteins and Combinations Thereof and Assessment of dCas9-VPR3

This example demonstrates that, in addition to the nine ZF-VPR proteins tested in Example 4, other ZF-VPR fusion proteins and various combinations thereof can upregulate the expression of HNF4α in LX-2 cells.

In the first experiment, fusion proteins were screened in LX-2 cells. Untreated Hep G2 cells were included as an assay control. LX-2 cells was treated with a single concentration of effector (2.5 μg/mL) in triplicate and incubated for 48 hours. ZF-5-VPR was used as a positive control for qPCR readout. LX-2 cells were transfected as described above using MC3-LNP formulations. The mRNAs encoding the following ZF-VPR fusion proteins and various combinations were transferred: ZF5-VPR, ZF5-VPR-ATUM3 (ZF5.3-VPR), ZF-7-VPR, ZF-11-VPR, ZF-13-VPR, and ZF-15-VPR, or combinations thereof.

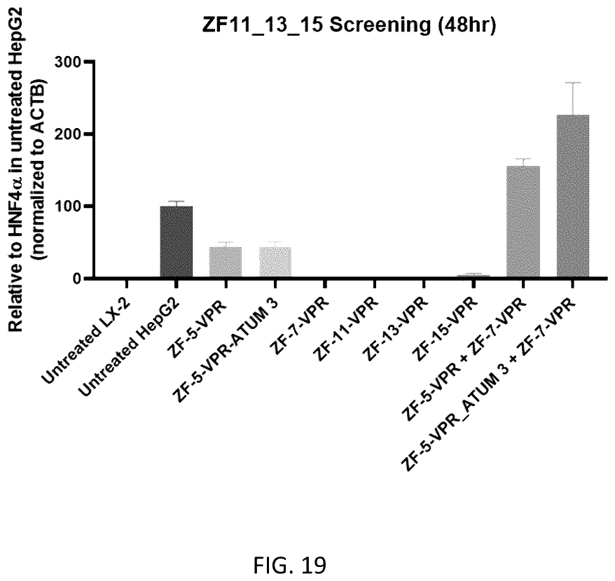

As shown in FIG. 19, stronger upregulation was observed when ZF5-VPR or ZF5.3-VPR were combined with ZF7-VPR. ZF7-VPR alone upregulated HNF4α in LX-2 cells to a low level.

In the second experiment, activation of HNF4α using dCas9-VPR3-Pool 1 or ZF-VPR fusion protein combinations was assessed. The objective was to evaluate the effects of various ZF-VPR fusion proteins in combinations in LX-2 Cells and test the new effector dCas9-VPR3 on LX-2 cells. VPR3 is a dCas9 DNA binding domain fused to 3 consecutive VPR domains in a row, expressed as a single protein. The combinations of ZF5.3-VPR and other ZF-VPRs that slightly upregulated the expression of HNF4α in LX-2 cells were tested for possible synergy. LX-2 cells were treated with a single concentration of effectors (2.5 ag/mL) in MC3 LNP formulations in triplicate for 48 hours. ZF-5-VPR was used as a positive control qPCR readout was used. The following combinations were tested: ZF5.3-VPR and ZF10-VPR, ZF5.3-VPR and ZF14-VPR, and ZF5.3-VPR and ZF15-VPR.

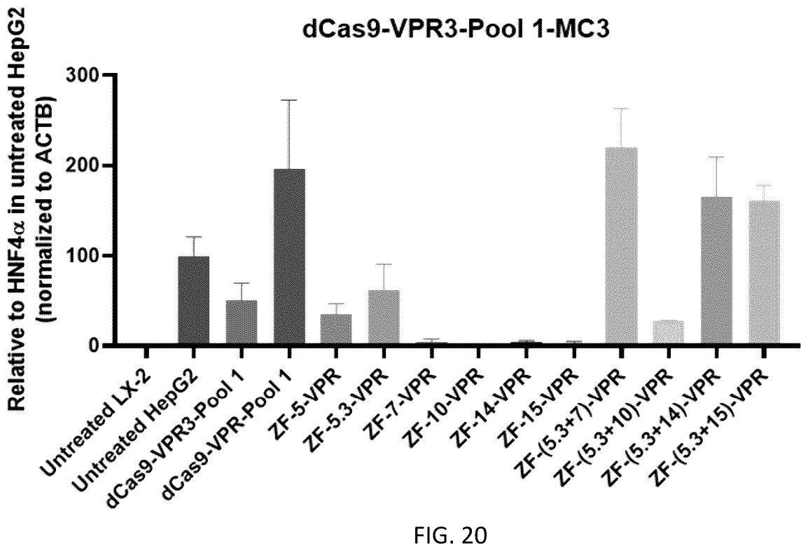

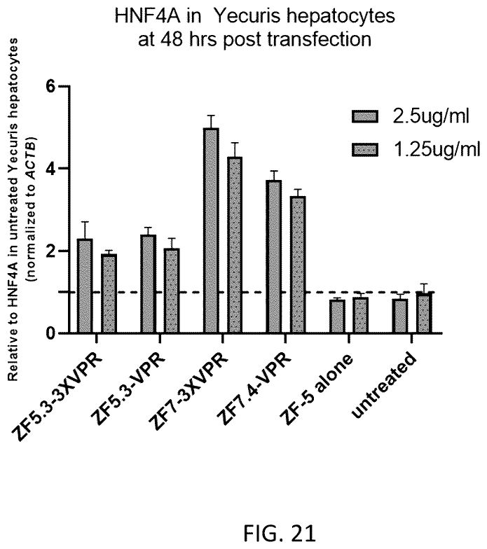

As shown in FIG. 20, dCas9-VPR3 Pool 1 upregulates HNF4α in LX-2 cells. ZF14-VPR and ZF15-VPR caused low upregulation of HNF4α when transfected individually. A strong synergistic upregulation (similar to the synergistic upregulation observed with ZF5.3-VPR+ZF7-VPR) was observed when ZF14-VPR or ZF15-VPR were in combination with ZF5.3-VPR.

### Example 10. HNF4α Activation in FRG-KO Mouse Liver Humanized Hepatocytes (Yecuris Human Hepatocytes)

Yecuris human hepatocytes are primary human hepatocytes ex-planted into an immunocompromised mouse, allowed to proliferate, and then harvested for in vitro tissue culture. Yecuris hepatocytes were obtained as a cell suspension and plated in 96-well format at 40K cells/well. Cells were treated with ZF fusion protein-MC3 LNPs for 24 hrs at two concentrations, 2.5 μg/ml and 1.25 μg/ml. HNF4α gene expression was measured at 48 hrs post treatment. ZF7.4-VPR was used instead of ZF7-VPR.

Yecuris cells were transfected with mRNAs encoding ZF5-VPR, ZF5.3-VPR, ZF7.4-VPR, ZF5.3-VPR3 (a ZF5.3 protein fused to 3 consecutive VPR domains in a row, expressed as a single protein), ZF7.4-VPR3, and ZF5-alone (with no VPR fused to ZF5).

As shown in FIG. 21, ZF5.3-VPR3 does not increase HNF4α gene expression compared to ZF5.3-VPR. An increase in HNF4α gene expression was observed with ZF7-VPR3 as compared to ZF7.4-VPR. As compared to the LX-2 cells, the ZF7-VPR constructs upregulate HNF4α to a greater level.

### Example 11. Activation of LX-2 Cells with Bicistronic ZF5.3-VPR and ZF7-VPR

This example evaluates whether ZF5.3-VPR (ZF5-VPR ATUM variant 3) and ZF7-VPR bicistronic constructs upregulate HNF4α in LX-2 cells to the same level as ZF5.3-VPR and ZF7-VPR when individually combined.

Untreated Hep G2 cells were included as an assay control. LX-2 cells were treated with a single concentration of the mRNAs encoding the fusion proteins (2.5 μg/mL) in triplicate. The combination of ZF5.3-VPR and ZF7-VPR was used as a positive control. qPCR readout was used to measure mRNA expression.

The following bicistronic mRNA constructs were tested: ZF5.3-VPR-tPT2A-ZF7-VPR, ZF7-VPR-tPT2A-ZF5.3-VPR, ZF5.3-VPR-tPT2A-ZF7-p300, and ZF7-p300-tPT2A-ZF5.3-VPR (see Table 6A). tPT2A is a linker that covalently links a first fusion protein to a second fusion protein to generate a bicistronic fusion protein.

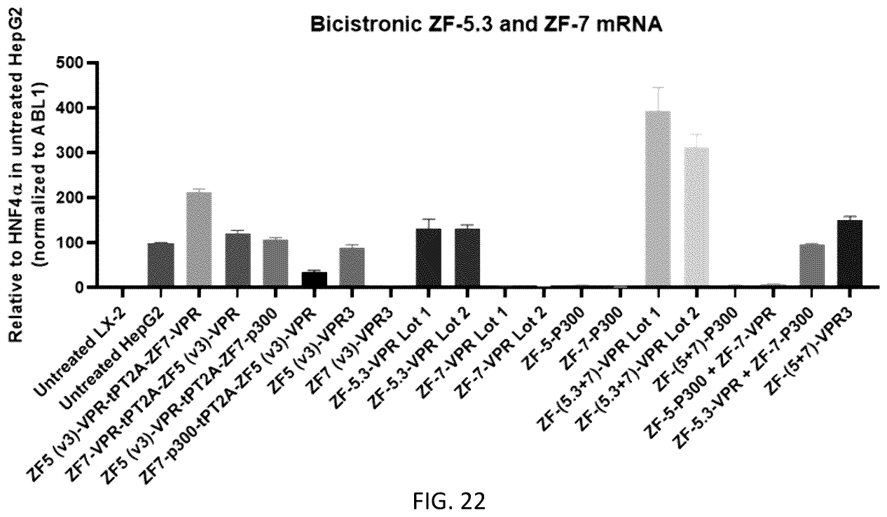

As shown in FIG. 22, the bicistronic mRNA ZF5.3-VPR-tPT2A-ZF7-VPR induced stronger upregulation of HNF4α in LX-2 cells than the other 3 bicistronic mRNAs tested.

### Example 12. Effect of Repeat Dosing of Yecuris Hepatocytes with VPR and p300 on HNF4α Gene Expression

This example describes the effect of delivering multiple doses of mRNA encoding ZF-effector fusion proteins to cells. The objective was to evaluate whether additive or synergistic upregulation of HNF4α can be achieved by multiple dosing.

Yecuris hepatocytes were plated at 64K cells/well. Cells were treated with the combination of ZF5.3-VPR and ZF7-VPR or ZF7-p300 via MC3 LNP formulations at a final concentration of 1.25 μg/ml. Bicistronic mRNAs ZF5.3-VPR-tPT2A-ZF7-VPR, ZF7-VPR-tPT2A-ZF5.3-VPR, ZF5.3-VPR-tPT2A-ZF7-p300, ZF7-p300-tPT2A-ZF5.3-VPR in MC3 LNP formulations were used to treat cells at a final concentration of 1.25 μg/ml.

The expression level of the HNF4α was measured at 48 hrs post last dose. The dosing and harvesting schedule followed in this experiment is provided in Table 8, below.

TABLE 8

| | Dose | Harvest |
|---|---|---|
| Day 0 | Dose 1 | |
| Day 1 | Media change | |
| Day 2 | | Harvest |
| Day 3 | Dose 2 | |
| Day 4 | Media change | |
| Day 5 | | Harvest |
| Day 6 | Dose 3 | |
| Day 7 | Media change | |
| Day 8 | | Harvest |

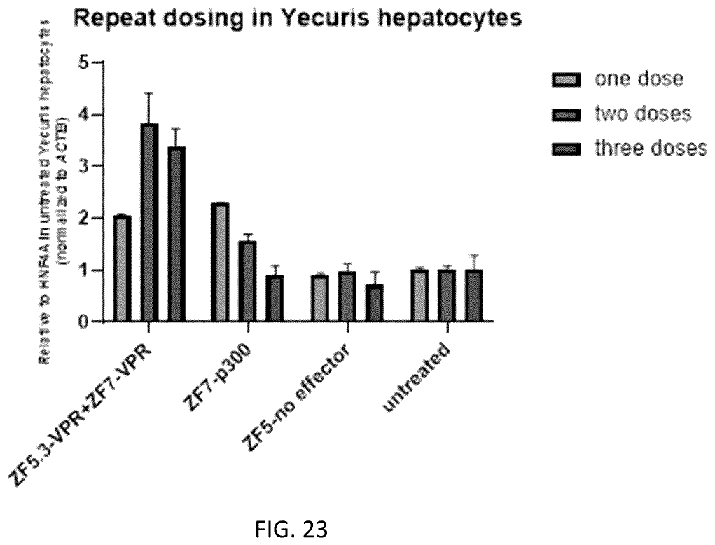

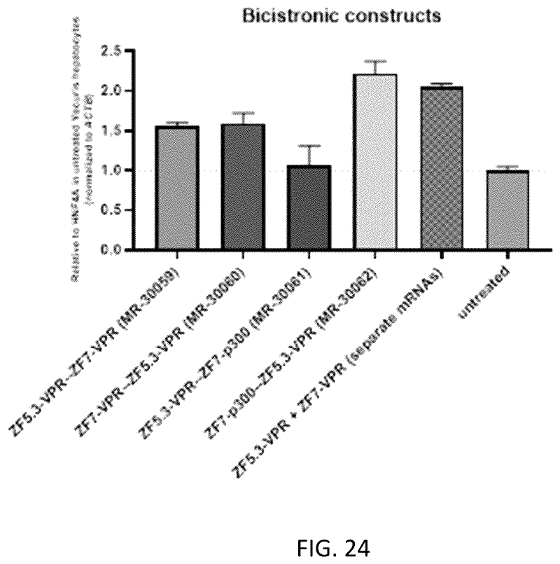

As shown in FIG. 23, repeated dosing of cells with MC3 LNP formulations containing mRNAs encoding ZF-effector fusion proteins resulted in an additive increase of expression of HNF4α. The combination of ZF5.3-VPR and ZF7-VPR was stronger than ZF7-p300 in its activation potential. In addition, and as shown in FIG. 24, bicistronic constructs increased HNF4α in Yecuris hepatocytes, with ZF7-p300-tPT2A-ZF5.3-VPR providing the strongest upregulation.

### Example 13: Activation of HNF4α in K562 Cells with Bi-cistronic mRNA-10 Day Durability Study

This example describes the durability of the bicistronic constructs in K562 cells. K562 cells were treated with a single concentration of mRNAs encoding the fusion proteins in MC3 LNPs (2.5 μg/mL) in triplicate as described above.

The bicistronic mRNAs encoding the following constructs were tested: ZF5.3-VPR-tPT2A-ZF7-VPR, ZF7-VPR-tPT2A-ZF5.3-VPR, ZF5.3-VPR-tPT2A-ZF7-p300, ZF7-p300-tPT2A-ZF5.3-VPR.

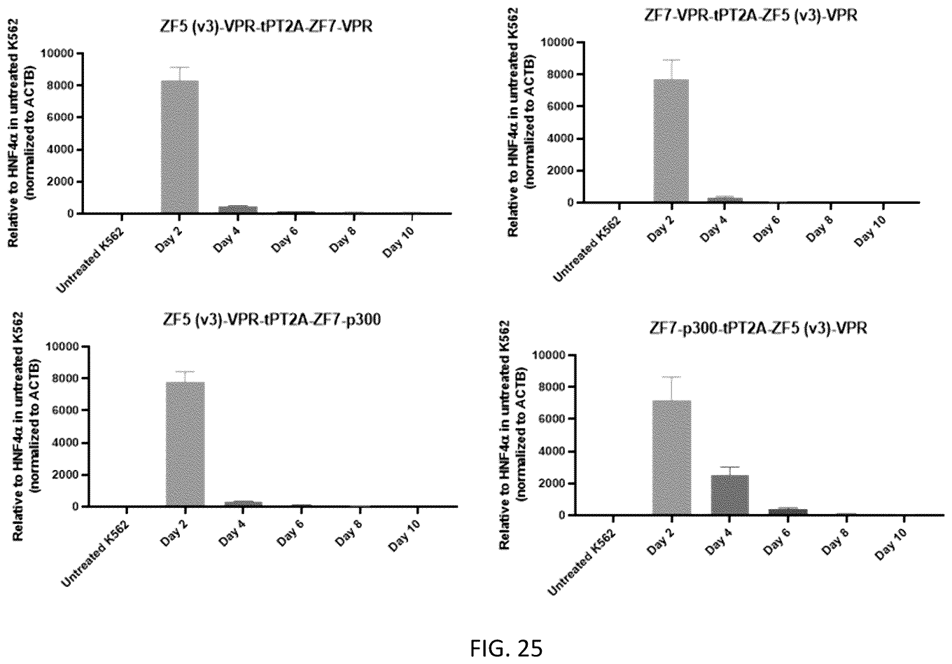

As shown in FIG. 25, ZF7-p300-tPT2A-ZF5 (v3)-VPR showed better durability in K562 at later days as compared to other bicistronic constructs.

TABLE 9

| Genomic Coordinates of | | | | | | |
|---|---|---|---|---|---|---|
| the Target Start Site in | | | | | | |
| Genome Reference Consortium | | | | | | |
| Human Build 37 (GRCh37): | | | | | sgRNA sequence | |
| Chromosome 20) | Strand | Target sequence | SEQ ID NO | PAM | [PLEASE ADVISE, NO “Us”] | SEQ ID NO |
| 43029193 | −1 | CCCTCACCCCCACCCCCTCC | 344 | CGG | CCCTCACCCCCACCCCCTCCGTTTTAGAGCTAGAAATAGCAAGTTA | 596 |
| | | | | | AAATAAGGCTAGTCCGTTATCAACTTGAAAAAGTGGCACCGAGTCG | |
| | | | | | GTGCTTTT | |
| 43029203 | 1 | TCCGGGAGGGGGTGGGGGTG | 345 | AGG | TCCGGGAGGGGGTGGGGGTGGTTTTAGAGCTAGAAATAGCAAGTTA | 597 |
| | | | | | AAATAAGGCTAGTCCGTTATCAACTTGAAAAAGTGGCACCGAGTCG | |
| | | | | | GTGCTTTT | |
| 43029204 | 1 | CCGGGAGGGGGTGGG | 346 | GGG | CCGGGAGGGGGTGGGGGTGAGTTTTAGAGCTAGAAATAGCAAGTTA | 598 |
| | | GGTGA | | | AAATAAGGCTAGTCCGTTATCAACTTGAAAAAGTGGCACCGAGTCG | |
| | | | | | GTGCTTTT | |
| 43029211 | 1 | GGGGTGGGGGTGAGG | 347 | AGG | GGGGTGGGGGTGAGGGAAACGTTTTAGAGCTAGAAATAGCAAGTTA | 599 |
| | | GAAAC | | | AAATAAGGCTAGTCCGTTATCAACTTGAAAAAGTGGCACCGAGTCG | |
| | | | | | GTGCTTTT | |
| 43029223 | 1 | AGGGAAACAGGAGAA | 348 | TGG | AGGGAAACAGGAGAATGTGAGTTTTAGAGCTAGAAATAGCAAGTTA | 600 |
| | | TGTGA | | | AAATAAGGCTAGTCCGTTATCAACTTGAAAAAGTGGCACCGAGTCG | |
| | | | | | GTGCTTTT | |
| 43029224 | 1 | GGGAAACAGGAGAAT | 349 | GGG | GGGAAACAGGAGAATGTGATGTTTTAGAGCTAGAAATAGCAAGTTA | 601 |
| | | GTGAT | | | AAATAAGGCTAGTCCGTTATCAACTTGAAAAAGTGGCACCGAGTCG | |
| | | | | | GTGCTTTT | |
| 43029238 | 1 | TGTGATGGGAAAATC | 350 | TGG | TGTGATGGGAAAATCCGAGAGTTTTAGAGCTAGAAATAGCAAGTTA | 602 |
| | | CGAGA | | | AAATAAGGCTAGTCCGTTATCAACTTGAAAAAGTGGCACCGAGTCG | |
| | | | | | GTGCTTTT | |
| 43029241 | −1 | GGCCCAGGCTGGCTC | 351 | CGG | GGCCCAGGCTGGCTCCATCTGTTTTAGAGCTAGAAATAGCAAGTTA | 603 |
| | | CATCT | | | AAATAAGGCTAGTCCGTTATCAACTTGAAAAAGTGGCACCGAGTCG | |
| | | | | | GTGCTTTT | |
| 43029249 | 1 | AATCCGAGATGGAGC | 352 | TGG | AATCCGAGATGGAGCCAGCCGTTTTAGAGCTAGAAATAGCAAGTTA | 604 |
| | | CAGCC | | | AAATAAGGCTAGTCCGTTATCAACTTGAAAAAGTGGCACCGAGTCG | |
| | | | | | GTGCTTTT | |
| 43029250 | 1 | ATCCGAGATGGAGCC | 353 | GGG | ATCCGAGATGGAGCCAGCCTGTTTTAGAGCTAGAAATAGCAAGTTA | 605 |
| | | AGCCT | | | AAATAAGGCTAGTCCGTTATCAACTTGAAAAAGTGGCACCGAGTCG | |
| | | | | | GTGCTTTT | |
| 43029252 | −1 | CCAGTGTTTCTGGCC | 354 | TGG | CCAGTGTTTCTGGCCCAGGCGTTTTAGAGCTAGAAATAGCAAGTTA | 606 |
| | | CAGGC | | | AAATAAGGCTAGTCCGTTATCAACTTGAAAAAGTGGCACCGAGTCG | |
| | | | | | GTGCTTTT | |
| 43029256 | −1 | GCTCCCAGTGTTTCT | 355 | AGG | GCTCCCAGTGTTTCTGGCCCGTTTTAGAGCTAGAAATAGCAAGTTA | 607 |
| | | GGCCC | | | AAATAAGGCTAGTCCGTTATCAACTTGAAAAAGTGGCACCGAGTCG | |
| | | | | | GTGCTTTT | |
| 43029262 | −1 | CCCACAGCTCCCAGT | 356 | TGG | CCCACAGCTCCCAGTGTTTCGTTTTAGAGCTAGAAATAGCAAGTTA | 608 |
| | | GTTTC | | | AAATAAGGCTAGTCCGTTATCAACTTGAAAAAGTGGCACCGAGTCG | |
| | | | | | GTGCTTTT | |
| 43029263 | 1 | CCAGCCTGGGCCAGA | 357 | TGG | CCAGCCTGGGCCAGAAACACGTTTTAGAGCTAGAAATAGCAAGTTA | 609 |
| | | AACAC | | | AAATAAGGCTAGTCCGTTATCAACTTGAAAAAGTGGCACCGAGTCG | |
| | | | | | GTGCTTTT | |
| 43029264 | 1 | CAGCCTGGGCCAGAA | 358 | GGG | CAGCCTGGGCCAGAAACACTGTTTTAGAGCTAGAAATAGCAAGTTA | 610 |
| | | ACACT | | | AAATAAGGCTAGTCCGTTATCAACTTGAAAAAGTGGCACCGAGTCG | |
| | | | | | GTGCTTTT | |
| 43029272 | 1 | GCCAGAAACACTGGG | 359 | TGG | GCCAGAAACACTGGGAGCTGGTTTTAGAGCTAGAAATAGCAAGTTA | 611 |
| | | AGCTG | | | AAATAAGGCTAGTCCGTTATCAACTTGAAAAAGTGGCACCGAGTCG | |
| | | | | | GTGCTTTT | |
| 43029273 | 1 | CCAGAAACACTGGGA | 360 | GGG | CCAGAAACACTGGGAGCTGTGTTTTAGAGCTAGAAATAGCAAGTTA | 612 |
| | | GCTGT | | | AAATAAGGCTAGTCCGTTATCAACTTGAAAAAGTGGCACCGAGTCG | |
| | | | | | GTGCTTTT | |
| 43029279 | 1 | ACACTGGGAGCTGTGGGAGA | 361 | CGG | ACACTGGGAGCTGTGGGAGAGTTTTAGAGCTAGAAATAGCAAGTTA | 613 |
| | | | | | AAATAAGGCTAGTCCGTTATCAACTTGAAAAAGTGGCACCGAGTCG | |
| | | | | | GTGCTTTT | |
| 43029284 | 1 | GGGAGCTGTGGGAGA | 362 | AGG | GGGAGCTGTGGGAGACGGAGGTTTTAGAGCTAGAAATAGCAAGTTA | 614 |
| | | CGGAG | | | AAATAAGGCTAGTCCGTTATCAACTTGAAAAAGTGGCACCGAGTCG | |
| | | | | | GTGCTTTT | |
| 43029285 | 1 | GGAGCTGTGGGAGAC | 363 | GGG | GGAGCTGTGGGAGACGGAGAGTTTTAGAGCTAGAAATAGCAAGTTA | 615 |
| | | GGAGA | | | AAATAAGGCTAGTCCGTTATCAACTTGAAAAAGTGGCACCGAGTCG | |
| | | | | | GTGCTTTT | |
| 43029286 | 1 | GAGCTGTGGGAGACG | 364 | GGG | GAGCTGTGGGAGACGGAGAGGTTTTAGAGCTAGAAATAGCAAGTTA | 616 |
| | | GAGAG | | | AAATAAGGCTAGTCCGTTATCAACTTGAAAAAGTGGCACCGAGTCG | |
| | | | | | GTGCTTTT | |
| 43029290 | 1 | TGTGGGAGACGGAGA | 365 | AGG | TGTGGGAGACGGAGAGGGGCGTTTTAGAGCTAGAAATAGCAAGTTA | 617 |
| | | GGGGC | | | AAATAAGGCTAGTCCGTTATCAACTTGAAAAAGTGGCACCGAGTCG | |
| | | | | | GTGCTTTT | |
| 43029291 | 1 | GTGGGAGACGGAGAG | 366 | GGG | GTGGGAGACGGAGAGGGGCAGTTTTAGAGCTAGAAATAGCAAGTTA | 618 |
| | | GGGCA | | | AAATAAGGCTAGTCCGTTATCAACTTGAAAAAGTGGCACCGAGTCG | |
| | | | | | GTGCTTTT | |
| 43029294 | 1 | GGAGACGGAGAGGGG | 367 | TGG | GGAGACGGAGAGGGGCAGGGGTTTTAGAGCTAGAAATAGCAAGTTA | 619 |
| | | CAGGG | | | AAATAAGGCTAGTCCGTTATCAACTTGAAAAAGTGGCACCGAGTCG | |
| | | | | | GTGCTTTT | |
| 43029295 | 1 | GAGACGGAGAGGGGC | 368 | GGG | GAGACGGAGAGGGGCAGGGTGTTTTAGAGCTAGAAATAGCAAGTTA | 620 |
| | | AGGGT | | | AAATAAGGCTAGTCCGTTATCAACTTGAAAAAGTGGCACCGAGTCG | |
| | | | | | GTGCTTTT | |
| 43029303 | 1 | GAGGGGCAGGGTGGG | 369 | AGG | GAGGGGCAGGGTGGGATCACGTTTTAGAGCTAGAAATAGCAAGTTA | 621 |
| | | ATCAC | | | AAATAAGGCTAGTCCGTTATCAACTTGAAAAAGTGGCACCGAGTCG | |
| | | | | | GTGCTTTT | |
| 43029304 | 1 | AGGGGCAGGGTGGGA | 370 | GGG | AGGGGCAGGGTGGGATCACAGTTTTAGAGCTAGAAATAGCAAGTTA | 622 |
| | | TCACA | | | AAATAAGGCTAGTCCGTTATCAACTTGAAAAAGTGGCACCGAGTCG | |
| | | | | | GTGCTTTT | |
| 43029310 | 1 | AGGGTGGGATCACAGGGAGC | 371 | AGG | AGGGTGGGATCACAGGGAGCGTTTTAGAGCTAGAAATAGCAAGTTA | 623 |
| | | | | | AAATAAGGCTAGTCCGTTATCAACTTGAAAAAGTGGCACCGAGTCG | |
| | | | | | GTGCTTTT | |
| 43029315 | 1 | GGGATCACAGGGAGC | 372 | CGG | GGGATCACAGGGAGCAGGAGGTTTTAGAGCTAGAAATAGCAAGTTA | 624 |
| | | AGGAG | | | AAATAAGGCTAGTCCGTTATCAACTTGAAAAAGTGGCACCGAGTCG | |
| | | | | | GTGCTTTT | |
| 43029316 | 1 | GGATCACAGGGAGCA | 373 | GGG | GGATCACAGGGAGCAGGAGCGTTTTAGAGCTAGAAATAGCAAGTTA | 625 |
| | | GGAGC | | | AAATAAGGCTAGTCCGTTATCAACTTGAAAAAGTGGCACCGAGTCG | |
| | | | | | GTGCTTTT | |
| 43029317 | 1 | GATCACAGGGAGCAG | 374 | GGG | GATCACAGGGAGCAGGAGCGGTTTTAGAGCTAGAAATAGCAAGTTA | 626 |
| | | GAGCG | | | AAATAAGGCTAGTCCGTTATCAACTTGAAAAAGTGGCACCGAGTCG | |
| | | | | | GTGCTTTT | |
| 43029323 | 1 | AGGGAGCAGGAGCGG | 375 | TGG | AGGGAGCAGGAGCGGGGAATGTTTTAGAGCTAGAAATAGCAAGTTA | 627 |
| | | GGAAT | | | AAATAAGGCTAGTCCGTTATCAACTTGAAAAAGTGGCACCGAGTCG | |
| | | | | | GTGCTTTT | |
| 43029326 | 1 | GAGCAGGAGCGGGGA | 376 | AGG | GAGCAGGAGCGGGGAATTGGGTTTTAGAGCTAGAAATAGCAAGTTA | 628 |
| | | ATTGG | | | AAATAAGGCTAGTCCGTTATCAACTTGAAAAAGTGGCACCGAGTCG | |
| | | | | | GTGCTTTT | |
| 43029335 | 1 | CGGGGAATTGGAGGT | 377 | TGG | CGGGGAATTGGAGGTGAATCGTTTTAGAGCTAGAAATAGCAAGTTA | 629 |
| | | | | | AAATAAGGCTAGTCCGTTATCAACTTGAAAAAGTGGCACCGAGTCG | |
| | | GAATC | | | GTGCTTTT | |
| 43029347 | −1 | AATGGACTGGAAGTT | 378 | GGG | AATGGACTGGAAGTTTGGGAGTTTTAGAGCTAGAAATAGCAAGTTA | 630 |
| | | TGGGA | | | AAATAAGGCTAGTCCGTTATCAACTTGAAAAAGTGGCACCGAGTCG | |
| | | | | | GTGCTTTT | |
| 43029348 | −1 | GAATGGACTGGAAGT | 379 | AGG | GAATGGACTGGAAGTTTGGGGTTTTAGAGCTAGAAATAGCAAGTTA | 631 |
| | | TTGGG | | | AAATAAGGCTAGTCCGTTATCAACTTGAAAAAGTGGCACCGAGTCG | |
| | | | | | GTGCTTTT | |
| 43029351 | −1 | GCAGAATGGACTGGA | 380 | GGG | GCAGAATGGACTGGAAGTTTGTTTTAGAGCTAGAAATAGCAAGTTA | 632 |
| | | AGTTT | | | AAATAAGGCTAGTCCGTTATCAACTTGAAAAAGTGGCACCGAGTCG | |
| | | | | | GTGCTTTT | |
| 43029352 | −1 | AGCAGAATGGACTGGAAGTT | 381 | TGG | AGCAGAATGGACTGGAAGTTGTTTTAGAGCTAGAAATAGCAAGTTA | 633 |
| | | | | | AAATAAGGCTAGTCCGTTATCAACTTGAAAAAGTGGCACCGAGTCG | |
| | | | | | GTGCTTTT | |
| 43029360 | −1 | CCCCTGGGAGCAGAA | 382 | TGG | CCCCTGGGAGCAGAATGGACGTTTTAGAGCTAGAAATAGCAAGTTA | 634 |
| | | TGGAC | | | AAATAAGGCTAGTCCGTTATCAACTTGAAAAAGTGGCACCGAGTCG | |
| | | | | | GTGCTTTT | |
| 43029365 | −1 | CGGTTCCCCTGGGAG | 383 | TGG | CGGTTCCCCTGGGAGCAGAAGTTTTAGAGCTAGAAATAGCAAGTTA | 635 |
| | | CAGAA | | | AAATAAGGCTAGTCCGTTATCAACTTGAAAAAGTGGCACCGAGTCG | |
| | | | | | GTGCTTTT | |
| 43029369 | 1 | TTCCAGTCCATTCTG | 384 | AGG | TTCCAGTCCATTCTGCTCCCGTTTTAGAGCTAGAAATAGCAAGTTA | 636 |
| | | CTCCC | | | AAATAAGGCTAGTCCGTTATCAACTTGAAAAAGTGGCACCGAGTCG | |
| | | | | | GTGCTTTT | |
| 43029370 | 1 | TCCAGTCCATTCTGC | 385 | GGG | TCCAGTCCATTCTGCTCCCAGTTTTAGAGCTAGAAATAGCAAGTTA | 637 |
| | | TCCCA | | | AAATAAGGCTAGTCCGTTATCAACTTGAAAAAGTGGCACCGAGTCG | |
| | | | | | GTGCTTTT | |
| 43029371 | 1 | CCAGTCCATTCTGCT | 386 | GGG | CCAGTCCATTCTGCTCCCAGGTTTTAGAGCTAGAAATAGCAAGTTA | 638 |
| | | CCCAG | | | AAATAAGGCTAGTCCGTTATCAACTTGAAAAAGTGGCACCGAGTCG | |
| | | | | | GTGCTTTT | |
| 43029375 | −1 | CGCAGTTTCCCGGTT | 387 | GGG | CGCAGTTTCCCGGTTCCCCTGTTTTAGAGCTAGAAATAGCAAGTTA | 639 |
| | | CCCCT | | | AAATAAGGCTAGTCCGTTATCAACTTGAAAAAGTGGCACCGAGTCG | |
| | | | | | GTGCTTTT | |
| 43029376 | −1 | CCGCAGTTTCCCGGT | 388 | TGG | CCGCAGTTTCCCGGTTCCCCGTTTTAGAGCTAGAAATAGCAAGTTA | 640 |
| | | TCCCC | | | AAATAAGGCTAGTCCGTTATCAACTTGAAAAAGTGGCACCGAGTCG | |
| | | | | | GTGCTTTT | |
| 43029377 | 1 | CATTCTGCTCCCAGG | 389 | CGG | CATTCTGCTCCCAGGGGAACGTTTTAGAGCTAGAAATAGCAAGTTA | 641 |
| | | GGAAC | | | AAATAAGGCTAGTCCGTTATCAACTTGAAAAAGTGGCACCGAGTCG | |
| | | | | | GTGCTTTT | |
| 43029378 | 1 | ATTCTGCTCCCAGGG | 390 | GGG | ATTCTGCTCCCAGGGGAACCGTTTTAGAGCTAGAAATAGCAAGTTA | 642 |
| | | GAACC | | | AAATAAGGCTAGTCCGTTATCAACTTGAAAAAGTGGCACCGAGTCG | |
| | | | | | GTGCTTTT | |
| 43029385 | −1 | CCAGTTCCCCCGCAG | 391 | CGG | CCAGTTCCCCCGCAGTTTCCGTTTTAGAGCTAGAAATAGCAAGTTA | 643 |
| | | TTTCC | | | AAATAAGGCTAGTCCGTTATCAACTTGAAAAAGTGGCACCGAGTCG | |
| | | | | | GTGCTTTT | |
| 43029387 | 1 | CCAGGGGAACCGGGA | 392 | CGG | CCAGGGGAACCGGGAAACTGGTTTTAGAGCTAGAAATAGCAAGTTA | 644 |
| | | AACTG | | | AAATAAGGCTAGTCCGTTATCAACTTGAAAAAGTGGCACCGAGTCG | |
| | | | | | GTGCTTTT | |
| 43029388 | 1 | CAGGGGAACCGGGAA | 393 | GGG | CAGGGGAACCGGGAAACTGCGTTTTAGAGCTAGAAATAGCAAGTTA | 645 |
| | | ACTGC | | | AAATAAGGCTAGTCCGTTATCAACTTGAAAAAGTGGCACCGAGTCG | |
| | | | | | GTGCTTTT | |
| 43029389 | 1 | AGGGGAACCGGGAAA | 394 | GGG | AGGGGAACCGGGAAACTGCGGTTTTAGAGCTAGAAATAGCAAGTTA | 646 |
| | | CTGCG | | | AAATAAGGCTAGTCCGTTATCAACTTGAAAAAGTGGCACCGAGTCG | |
| | | | | | GTGCTTTT | |
| 43029390 | 1 | GGGGAACCGGGAAAC | 395 | GGG | GGGGAACCGGGAAACTGCGGGTTTTAGAGCTAGAAATAGCAAGTTA | 647 |
| | | TGCGG | | | AAATAAGGCTAGTCCGTTATCAACTTGAAAAAGTGGCACCGAGTCG | |
| | | | | | GTGCTTTT | |
| 43029396 | 1 | CCGGGAAACTGCGGG | 396 | TGG | CCGGGAAACTGCGGGGGAACGTTTTAGAGCTAGAAATAGCAAGTTA | 648 |
| | | GGAAC | | | AAATAAGGCTAGTCCGTTATCAACTTGAAAAAGTGGCACCGAGTCG | |
| | | | | | GTGCTTTT | |
| 43029400 | 1 | GAAACTGCGGGGGAA | 397 | AGG | GAAACTGCGGGGGAACTGGAGTTTTAGAGCTAGAAATAGCAAGTTA | 649 |
| | | CTGGA | | | AAATAAGGCTAGTCCGTTATCAACTTGAAAAAGTGGCACCGAGTCG | |
| | | | | | GTGCTTTT | |
| 43029401 | 1 | AAACTGCGGGGGAAC | 398 | GGG | AAACTGCGGGGGAACTGGAAGTTTTAGAGCTAGAAATAGCAAGTTA | 650 |
| | | TGGAA | | | AAATAAGGCTAGTCCGTTATCAACTTGAAAAAGTGGCACCGAGTCG | |
| | | | | | GTGCTTTT | |
| 43029417 | 1 | GGAAGGGAGCTCCCA | 399 | AGG | GGAAGGGAGCTCCCAGAACAGTTTTAGAGCTAGAAATAGCAAGTTA | 651 |
| | | GAACA | | | AAATAAGGCTAGTCCGTTATCAACTTGAAAAAGTGGCACCGAGTCG | |
| | | | | | GTGCTTTT | |
| 43029417 | −1 | ATCTTCTGGATCCTT | 400 | GGG | ATCTTCTGGATCCTTGTTCTGTTTTAGAGCTAGAAATAGCAAGTTA | 652 |
| | | GTTCT | | | AAATAAGGCTAGTCCGTTATCAACTTGAAAAAGTGGCACCGAGTCG | |
| | | | | | GTGCTTTT | |
| 43029418 | −1 | AATCTTCTGGATCCTTGTTC | 401 | TGG | AATCTTCTGGATCCTTGTTCGTTTTAGAGCTAGAAATAGCAAGTTA | 653 |
| | | | | | AAATAAGGCTAGTCCGTTATCAACTTGAAAAAGTGGCACCGAGTCG | |
| | | | | | GTGCTTTT | |
| 43029431 | 1 | AGAACAAGGATCCAG | 402 | TGG | AGAACAAGGATCCAGAAGATGTTTTAGAGCTAGAAATAGCAAGTTA | 654 |
| | | AAGAT | | | AAATAAGGCTAGTCCGTTATCAACTTGAAAAAGTGGCACCGAGTCG | |
| | | | | | GTGCTTTT | |
| 43029431 | −1 | GGCCCCAGATGCCAA | 403 | TGG | GGCCCCAGATGCCAATCTTCGTTTTAGAGCTAGAAATAGCAAGTTA | 655 |
| | | TCTTC | | | AAATAAGGCTAGTCCGTTATCAACTTGAAAAAGTGGCACCGAGTCG | |
| | | | | | GTGCTTTT | |
| 43029438 | 1 | GGATCCAGAAGATTG | 404 | TGG | GGATCCAGAAGATTGGCATCGTTTTAGAGCTAGAAATAGCAAGTTA | 656 |
| | | GCATC | | | AAATAAGGCTAGTCCGTTATCAACTTGAAAAAGTGGCACCGAGTCG | |
| | | | | | GTGCTTTT | |
| 43029439 | 1 | GATCCAGAAGATTGG | 405 | GGG | GATCCAGAAGATTGGCATCTGTTTTAGAGCTAGAAATAGCAAGTTA | 657 |
| | | CATCT | | | AAATAAGGCTAGTCCGTTATCAACTTGAAAAAGTGGCACCGAGTCG | |
| | | | | | GTGCTTTT | |
| 43029440 | 1 | ATCCAGAAGATTGGC | 406 | GGG | ATCCAGAAGATTGGCATCTGGTTTTAGAGCTAGAAATAGCAAGTTA | 658 |
| | | ATCTG | | | AAATAAGGCTAGTCCGTTATCAACTTGAAAAAGTGGCACCGAGTCG | |
| | | | | | GTGCTTTT | |
| 43029445 | 1 | GAAGATTGGCATCTG | 407 | TGG | GAAGATTGGCATCTGGGGCCGTTTTAGAGCTAGAAATAGCAAGTTA | 659 |
| | | GGGCC | | | AAATAAGGCTAGTCCGTTATCAACTTGAAAAAGTGGCACCGAGTCG | |
| | | | | | GTGCTTTT | |
| 43029446 | 1 | AAGATTGGCATCTGG | 408 | GGG | AAGATTGGCATCTGGGGCCTGTTTTAGAGCTAGAAATAGCAAGTTA | 660 |
| | | GGCCT | | | AAATAAGGCTAGTCCGTTATCAACTTGAAAAAGTGGCACCGAGTCG | |
| | | | | | GTGCTTTT | |
| 43029452 | −1 | GATTTAGAAACCTAA | 409 | AGG | GATTTAGAAACCTAAATCCCGTTTTAGAGCTAGAAATAGCAAGTTA | 661 |
| | | ATCCC | | | AAATAAGGCTAGTCCGTTATCAACTTGAAAAAGTGGCACCGAGTCG | |
| | | | | | GTGCTTTT | |
| 43029453 | 1 | GCATCTGGGGCCTGG | 410 | AGG | GCATCTGGGGCCTGGGATTTGTTTTAGAGCTAGAAATAGCAAGTTA | 662 |
| | | GATTT | | | AAATAAGGCTAGTCCGTTATCAACTTGAAAAAGTGGCACCGAGTCG | |
| | | | | | GTGCTTTT | |
| 43029467 | 1 | GGATTTAGGTTTCTA | 411 | TGG | GGATTTAGGTTTCTAAATCGGTTTTAGAGCTAGAAATAGCAAGTTA | 663 |
| | | AATCG | | | AAATAAGGCTAGTCCGTTATCAACTTGAAAAAGTGGCACCGAGTCG | |
| | | | | | GTGCTTTT | |
| 43029468 | 1 | GATTTAGGTTTCTAA | 412 | GGG | GATTTAGGTTTCTAAATCGTGTTTTAGAGCTAGAAATAGCAAGTTA | 664 |
| | | ATCGT | | | AAATAAGGCTAGTCCGTTATCAACTTGAAAAAGTGGCACCGAGTCG | |
| | | | | | GTGCTTTT | |
| 43029474 | 1 | GGTTTCTAAATCGTG | 413 | TGG | GGTTTCTAAATCGTGGGCCAGTTTTAGAGCTAGAAATAGCAAGTTA | 665 |
| | | GGCCA | | | AAATAAGGCTAGTCCGTTATCAACTTGAAAAAGTGGCACCGAGTCG | |
| | | | | | GTGCTTTT | |
| 43029475 | 1 | GTTTCTAAATCGTGG | 414 | GGG | GTTTCTAAATCGTGGGCCATGTTTTAGAGCTAGAAATAGCAAGTTA | 666 |
| | | GCCAT | | | AAATAAGGCTAGTCCGTTATCAACTTGAAAAAGTGGCACCGAGTCG | |
| | | | | | GTGCTTTT | |
| 43029476 | 1 | TTTCTAAATCGTGGG | 415 | GGG | TTTCTAAATCGTGGGCCATGGTTTTAGAGCTAGAAATAGCAAGTTA | 667 |
| | | CCATG | | | AAATAAGGCTAGTCCGTTATCAACTTGAAAAAGTGGCACCGAGTCG | |
| | | | | | GTGCTTTT | |
| 43029480 | −1 | GCAGAGATAAGGCTG | 416 | TGG | GCAGAGATAAGGCTGCCCCAGTTTTAGAGCTAGAAATAGCAAGTTA | 668 |
| | | CCCCA | | | AAATAAGGCTAGTCCGTTATCAACTTGAAAAAGTGGCACCGAGTCG | |
| | | | | | GTGCTTTT | |
| 43029491 | −1 | TCAATGCTTTTGCAG | 417 | AGG | TCAATGCTTTTGCAGAGATAGTTTTAGAGCTAGAAATAGCAAGTTA | 669 |
| | | AGATA | | | AAATAAGGCTAGTCCGTTATCAACTTGAAAAAGTGGCACCGAGTCG | |
| | | | | | GTGCTTTT | |
| 43029504 | 1 | TTATCTCTGCAAAAG | 418 | AGG | TTATCTCTGCAAAAGCATTGGTTTTAGAGCTAGAAATAGCAAGTTA | 670 |
| | | CATTG | | | AAATAAGGCTAGTCCGTTATCAACTTGAAAAAGTGGCACCGAGTCG | |
| | | | | | GTGCTTTT | |
| 43029505 | 1 | TATCTCTGCAAAAGC | 419 | GGG | TATCTCTGCAAAAGCATTGAGTTTTAGAGCTAGAAATAGCAAGTTA | 671 |
| | | ATTGA | | | AAATAAGGCTAGTCCGTTATCAACTTGAAAAAGTGGCACCGAGTCG | |
| | | | | | GTGCTTTT | |
| 43029523 | 1 | GAGGGTAGAAGTCAA | 420 | TGG | GAGGGTAGAAGTCAATGATTGTTTTAGAGCTAGAAATAGCAAGTTA | 672 |
| | | TGATT | | | AAATAAGGCTAGTCCGTTATCAACTTGAAAAAGTGGCACCGAGTCG | |
| | | | | | GTGCTTTT | |
| 43029524 | 1 | AGGGTAGAAGTCAATGATTT | 421 | GGG | AGGGTAGAAGTCAATGATTTGTTTTAGAGCTAGAAATAGCAAGTTA | 673 |
| | | | | | AAATAAGGCTAGTCCGTTATCAACTTGAAAAAGTGGCACCGAGTCG | |
| | | | | | GTGCTTTT | |
| 43029540 | 1 | ATTTGGGAAGTTATT | 422 | AGG | ATTTGGGAAGTTATTGAATTGTTTTAGAGCTAGAAATAGCAAGTTA | 674 |
| | | GAATT | | | AAATAAGGCTAGTCCGTTATCAACTTGAAAAAGTGGCACCGAGTCG | |
| | | | | | GTGCTTTT | |
| 43029541 | 1 | TTTGGGAAGTTATTG | 423 | GGG | TTTGGGAAGTTATTGAATTAGTTTTAGAGCTAGAAATAGCAAGTTA | 675 |
| | | AATTA | | | AAATAAGGCTAGTCCGTTATCAACTTGAAAAAGTGGCACCGAGTCG | |
| | | | | | GTGCTTTT | |
| 43029542 | 1 | TTGGGAAGTTATTGA | 424 | GGG | TTGGGAAGTTATTGAATTAGGTTTTAGAGCTAGAAATAGCAAGTTA | 676 |
| | | ATTAG | | | AAATAAGGCTAGTCCGTTATCAACTTGAAAAAGTGGCACCGAGTCG | |
| | | | | | GTGCTTTT | |
| 43029549 | 1 | GTTATTGAATTAGGG | 425 | CGG | GTTATTGAATTAGGGGATCTGTTTTAGAGCTAGAAATAGCAAGTTA | 677 |
| | | GATCT | | | AAATAAGGCTAGTCCGTTATCAACTTGAAAAAGTGGCACCGAGTCG | |
| | | | | | GTGCTTTT | |
| 43029552 | 1 | ATTGAATTAGGGGAT | 333 | AGG | ATTGAATTAGGGGATCTCGGGTTTTAGAGCTAGAAATAGCAAGTTA | 678 |
| | | CTCGG | | | AAATAAGGCTAGTCCGTTATCAACTTGAAAAAGTGGCACCGAGTCG | |
| | | | | | GTGCTTTT | |
| 43029556 | 1 | AATTAGGGGATCTCG | 426 | AGG | AATTAGGGGATCTCGGAGGTGTTTTAGAGCTAGAAATAGCAAGTTA | 679 |
| | | GAGGT | | | AAATAAGGCTAGTCCGTTATCAACTTGAAAAAGTGGCACCGAGTCG | |
| | | | | | GTGCTTTT | |
| 43029577 | −1 | GCATTCTAACTGATA | 427 | AGG | GCATTCTAACTGATACTATCGTTTTAGAGCTAGAAATAGCAAGTTA | 680 |
| | | CTATC | | | AAATAAGGCTAGTCCGTTATCAACTTGAAAAAGTGGCACCGAGTCG | |
| | | | | | GTGCTTTT | |
| 43029597 | 1 | ATCAGTTAGAATGCC | 428 | TGG | ATCAGTTAGAATGCCTGACTGTTTTAGAGCTAGAAATAGCAAGTTA | 681 |
| | | TGACT | | | AAATAAGGCTAGTCCGTTATCAACTTGAAAAAGTGGCACCGAGTCG | |
| | | | | | GTGCTTTT | |
| 43029598 | 1 | TCAGTTAGAATGCCT | 429 | GGG | TCAGTTAGAATGCCTGACTTGTTTTAGAGCTAGAAATAGCAAGTTA | 682 |
| | | GACTT | | | AAATAAGGCTAGTCCGTTATCAACTTGAAAAAGTGGCACCGAGTCG | |
| | | | | | GTGCTTTT | |
| 43029599 | 1 | CAGTTAGAATGCCTG | 430 | GGG | CAGTTAGAATGCCTGACTTGGTTTTAGAGCTAGAAATAGCAAGTTA | 683 |
| | | ACTTG | | | AAATAAGGCTAGTCCGTTATCAACTTGAAAAAGTGGCACCGAGTCG | |
| | | | | | GTGCTTTT | |
| 43029599 | −1 | AGCCATTGTCACCCC | 340 | AGG | AGCCATTGTCACCCCAAGTCGTTTTAGAGCTAGAAATAGCAAGTTA | 684 |
| | | AAGTC | | | AAATAAGGCTAGTCCGTTATCAACTTGAAAAAGTGGCACCGAGTCG | |
| | | | | | GTGCTTTT | |
| 43029608 | 1 | TGCCTGACTTGGGGT | 431 | TGG | TGCCTGACTTGGGGTGACAAGTTTTAGAGCTAGAAATAGCAAGTTA | 685 |
| | | GACAA | | | AAATAAGGCTAGTCCGTTATCAACTTGAAAAAGTGGCACCGAGTCG | |
| | | | | | GTGCTTTT | |
| 43029613 | 1 | GACTTGGGGTGACAA | 334 | TGG | GACTTGGGGTGACAATGGCTGTTTTAGAGCTAGAAATAGCAAGTTA | 686 |
| | | TGGCT | | | AAATAAGGCTAGTCCGTTATCAACTTGAAAAAGTGGCACCGAGTCG | |
| | | | | | GTGCTTTT | |
| 43029616 | 1 | TTGGGGTGACAATGG | 432 | AGG | TTGGGGTGACAATGGCTTGGGTTTTAGAGCTAGAAATAGCAAGTTA | 687 |
| | | CTTGG | | | AAATAAGGCTAGTCCGTTATCAACTTGAAAAAGTGGCACCGAGTCG | |
| | | | | | GTGCTTTT | |
| 43029617 | 1 | TGGGGTGACAATGGC | 433 | GGG | TGGGGTGACAATGGCTTGGAGTTTTAGAGCTAGAAATAGCAAGTTA | 688 |
| | | TTGGA | | | AAATAAGGCTAGTCCGTTATCAACTTGAAAAAGTGGCACCGAGTCG | |
| | | | | | GTGCTTTT | |
| 43029618 | 1 | GGGGTGACAATGGCT | 434 | GGG | GGGGTGACAATGGCTTGGAGGTTTTAGAGCTAGAAATAGCAAGTTA | 689 |
| | | TGGAG | | | AAATAAGGCTAGTCCGTTATCAACTTGAAAAAGTGGCACCGAGTCG | |
| | | | | | GTGCTTTT | |
| 43029621 | 1 | GTGACAATGGCTTGG | 435 | TGG | GTGACAATGGCTTGGAGGGGGTTTTAGAGCTAGAAATAGCAAGTTA | 690 |
| | | AGGGG | | | AAATAAGGCTAGTCCGTTATCAACTTGAAAAAGTGGCACCGAGTCG | |
| | | | | | GTGCTTTT | |
| 43029622 | 1 | TGACAATGGCTTGGA | 436 | GGG | TGACAATGGCTTGGAGGGGTGTTTTAGAGCTAGAAATAGCAAGTTA | 691 |
| | | GGGGT | | | AAATAAGGCTAGTCCGTTATCAACTTGAAAAAGTGGCACCGAGTCG | |
| | | | | | GTGCTTTT | |
| 43029632 | 1 | TTGGAGGGGTGGGTG | 437 | AGG | TTGGAGGGGTGGGTGAGTCAGTTTTAGAGCTAGAAATAGCAAGTTA | 692 |
| | | AGTCA | | | AAATAAGGCTAGTCCGTTATCAACTTGAAAAAGTGGCACCGAGTCG | |
| | | | | | GTGCTTTT | |
| 43029633 | 1 | TGGAGGGGTGGGTGA | 438 | GGG | TGGAGGGGTGGGTGAGTCAAGTTTTAGAGCTAGAAATAGCAAGTTA | 693 |
| | | GTCAA | | | AAATAAGGCTAGTCCGTTATCAACTTGAAAAAGTGGCACCGAGTCG | |
| | | | | | GTGCTTTT | |
| 43029656 | −1 | AGGCAGGCATCATGA | 439 | GGG | AGGCAGGCATCATGACTCACGTTTTAGAGCTAGAAATAGCAAGTTA | 694 |
| | | CTCAC | | | AAATAAGGCTAGTCCGTTATCAACTTGAAAAAGTGGCACCGAGTCG | |
| | | | | | GTGCTTTT | |
| 43029657 | −1 | AAGGCAGGCATCATG | 440 | CGG | AAGGCAGGCATCATGACTCAGTTTTAGAGCTAGAAATAGCAAGTTA | 695 |
| | | ACTCA | | | AAATAAGGCTAGTCCGTTATCAACTTGAAAAAGTGGCACCGAGTCG | |
| | | | | | GTGCTTTT | |
| 43029672 | −1 | AGTTATCAATTGTAC | 441 | AGG | AGTTATCAATTGTACAAGGCGTTTTAGAGCTAGAAATAGCAAGTTA | 696 |
| | | AAGGC | | | AAATAAGGCTAGTCCGTTATCAACTTGAAAAAGTGGCACCGAGTCG | |
| | | | | | GTGCTTTT | |
| 43029676 | −1 | GTTCAGTTATCAATT | 442 | AGG | GTTCAGTTATCAATTGTACAGTTTTAGAGCTAGAAATAGCAAGTTA | 697 |
| | | GTACA | | | AAATAAGGCTAGTCCGTTATCAACTTGAAAAAGTGGCACCGAGTCG | |
| | | | | | GTGCTTTT | |
| 43029692 | 1 | TACAATTGATAACTG | 443 | CGG | TACAATTGATAACTGAACATGTTTTAGAGCTAGAAATAGCAAGTTA | 698 |
| | | AACAT | | | AAATAAGGCTAGTCCGTTATCAACTTGAAAAAGTGGCACCGAGTCG | |
| | | | | | GTGCTTTT | |
| 43029701 | 1 | TAACTGAACATCGGT | 444 | AGG | TAACTGAACATCGGTGAGTTGTTTTAGAGCTAGAAATAGCAAGTTA | 699 |
| | | GAGTT | | | AAATAAGGCTAGTCCGTTATCAACTTGAAAAAGTGGCACCGAGTCG | |
| | | | | | GTGCTTTT | |
| 43029702 | 1 | AACTGAACATCGGTG | 2511 | GGG | AACTGAACATCGGTGAGTTAGTTTTAGAGCTAGAAATAGCAAGTTA | 700 |
| | | AGTTA | | | AAATAAGGCTAGTCCGTTATCAACTTGAAAAAGTGGCACCGAGTCG | |
| | | | | | GTGCTTTT | |
| 43029714 | −1 | GGTGCTAATTACAAC | 445 | GGG | GGTGCTAATTACAACTGCTGGTTTTAGAGCTAGAAATAGCAAGTTA | 701 |
| | | TGCTG | | | AAATAAGGCTAGTCCGTTATCAACTTGAAAAAGTGGCACCGAGTCG | |
| | | | | | GTGCTTTT | |
| 43029715 | −1 | GGGTGCTAATTACAA | 446 | GGG | GGGTGCTAATTACAACTGCTGTTTTAGAGCTAGAAATAGCAAGTTA | 702 |
| | | CTGCT | | | AAATAAGGCTAGTCCGTTATCAACTTGAAAAAGTGGCACCGAGTCG | |
| | | | | | GTGCTTTT | |
| 43029716 | −1 | GGGGTGCTAATTACA | 447 | TGG | GGGGTGCTAATTACAACTGCGTTTTAGAGCTAGAAATAGCAAGTTA | 703 |
| | | ACTGC | | | AAATAAGGCTAGTCCGTTATCAACTTGAAAAAGTGGCACCGAGTCG | |
| | | | | | GTGCTTTT | |
| 43029729 | 1 | AGCAGTTGTAATTAG | 448 | CGG | AGCAGTTGTAATTAGCACCCGTTTTAGAGCTAGAAATAGCAAGTTA | 704 |
| | | CACCC | | | AAATAAGGCTAGTCCGTTATCAACTTGAAAAAGTGGCACCGAGTCG | |
| | | | | | GTGCTTTT | |
| 43029730 | 1 | GCAGTTGTAATTAGC | 449 | GGG | GCAGTTGTAATTAGCACCCCGTTTTAGAGCTAGAAATAGCAAGTTA | 705 |
| | | ACCCC | | | AAATAAGGCTAGTCCGTTATCAACTTGAAAAAGTGGCACCGAGTCG | |
| | | | | | GTGCTTTT | |
| 43029735 | −1 | TGGTTTCTGGCTGAC | 2512 | GGG | TGGTTTCTGGCTGACACCCGGTTTTAGAGCTAGAAATAGCAAGTTA | 706 |
| | | ACCCG | | | AAATAAGGCTAGTCCGTTATCAACTTGAAAAAGTGGCACCGAGTCG | |
| | | | | | GTGCTTTT | |
| 43029736 | −1 | TTGGTTTCTGGCTGA | 450 | GGG | TTGGTTTCTGGCTGACACCCGTTTTAGAGCTAGAAATAGCAAGTTA | 707 |
| | | CACCC | | | AAATAAGGCTAGTCCGTTATCAACTTGAAAAAGTGGCACCGAGTCG | |
| | | | | | GTGCTTTT | |
| 43029737 | −1 | GTTGGTTTCTGGCTG | 451 | CGG | GTTGGTTTCTGGCTGACACCGTTTTAGAGCTAGAAATAGCAAGTTA | 708 |
| | | ACACC | | | AAATAAGGCTAGTCCGTTATCAACTTGAAAAAGTGGCACCGAGTCG | |
| | | | | | GTGCTTTT | |
| 43029748 | −1 | TTTGGCTGTTTGTTG | 452 | TGG | TTTGGCTGTTTGTTGGTTTCGTTTTAGAGCTAGAAATAGCAAGTTA | 709 |
| | | GTTTC | | | AAATAAGGCTAGTCCGTTATCAACTTGAAAAAGTGGCACCGAGTCG | |
| | | | | | GTGCTTTT | |
| 43029755 | −1 | GCAGGGATTTGGCTG | 453 | TGG | GCAGGGATTTGGCTGTTTGTGTTTTAGAGCTAGAAATAGCAAGTTA | 710 |
| | | TTTGT | | | AAATAAGGCTAGTCCGTTATCAACTTGAAAAAGTGGCACCGAGTCG | |
| | | | | | GTGCTTTT | |
| 43029766 | -1 | TGGGCGGGGCTGCAG | 454 | TGG | TGGGCGGGGCTGCAGGGATTGTTTTAGAGCTAGAAATAGCAAGTTA | 711 |
| | | GGATT | | | AAATAAGGCTAGTCCGTTATCAACTTGAAAAAGTGGCACCGAGTCG | |
| | | | | | GTGCTTTT | |
| 43029772 | −1 | ATAGGCTGGGCGGGG | 455 | GGG | ATAGGCTGGGCGGGGCTGCAGTTTTAGAGCTAGAAATAGCAAGTTA | 712 |
| | | CTGCA | | | AAATAAGGCTAGTCCGTTATCAACTTGAAAAAGTGGCACCGAGTCG | |
| | | | | | GTGCTTTT | |
| 43029773 | −1 | GATAGGCTGGGCGGG | 456 | AGG | GATAGGCTGGGCGGGGCTGCGTTTTAGAGCTAGAAATAGCAAGTTA | 713 |
| | | GCTGC | | | AAATAAGGCTAGTCCGTTATCAACTTGAAAAAGTGGCACCGAGTCG | |
| | | | | | GTGCTTTT | |
| 43029780 | −1 | GCCGGTGGATAGGCT | 457 | GGG | GCCGGTGGATAGGCTGGGCGGTTTTAGAGCTAGAAATAGCAAGTTA | 714 |
| | | GGGCG | | | AAATAAGGCTAGTCCGTTATCAACTTGAAAAAGTGGCACCGAGTCG | |
| | | | | | GTGCTTTT | |
| 43029781 | −1 | CGCCGGTGGATAGGC | 458 | GGG | CGCCGGTGGATAGGCTGGGCGTTTTAGAGCTAGAAATAGCAAGTTA | 715 |
| | | TGGGC | | | AAATAAGGCTAGTCCGTTATCAACTTGAAAAAGTGGCACCGAGTCG | |
| | | | | | GTGCTTTT | |
| 43029782 | −1 | CCGCCGGTGGATAGG | 459 | CGG | CCGCCGGTGGATAGGCTGGGGTTTTAGAGCTAGAAATAGCAAGTTA | 716 |
| | | CTGGG | | | AAATAAGGCTAGTCCGTTATCAACTTGAAAAAGTGGCACCGAGTCG | |
| | | | | | GTGCTTTT | |
| 43029785 | −1 | CCCCCGCCGGTGGAT | 460 | GGG | CCCCCGCCGGTGGATAGGCTGTTTTAGAGCTAGAAATAGCAAGTTA | 717 |
| | | AGGCT | | | AAATAAGGCTAGTCCGTTATCAACTTGAAAAAGTGGCACCGAGTCG | |
| | | | | | GTGCTTTT | |
| 43029786 | −1 | TCCCCCGCCGGTGGA | 461 | TGG | TCCCCCGCCGGTGGATAGGCGTTTTAGAGCTAGAAATAGCAAGTTA | 718 |
| | | TAGGC | | | AAATAAGGCTAGTCCGTTATCAACTTGAAAAAGTGGCACCGAGTCG | |
| | | | | | GTGCTTTT | |
| 43029790 | 1 | GCCCCGCCCAGCCTA | 462 | CGG | GCCCCGCCCAGCCTATCCACGTTTTAGAGCTAGAAATAGCAAGTTA | 719 |
| | | TCCAC | | | AAATAAGGCTAGTCCGTTATCAACTTGAAAAAGTGGCACCGAGTCG | |
| | | | | | GTGCTTTT | |
| 43029790 | −1 | TCGGTCCCCCGCCGG | 463 | AGG | TCGGTCCCCCGCCGGTGGATGTTTTAGAGCTAGAAATAGCAAGTTA | 720 |
| | | TGGAT | | | AAATAAGGCTAGTCCGTTATCAACTTGAAAAAGTGGCACCGAGTCG | |
| | | | | | GTGCTTTT | |
| 43029793 | 1 | CCGCCCAGCCTATCC | 464 | CGG | CCGCCCAGCCTATCCACCGGGTTTTAGAGCTAGAAATAGCAAGTTA | 721 |
| | | ACCGG | | | AAATAAGGCTAGTCCGTTATCAACTTGAAAAAGTGGCACCGAGTCG | |
| | | | | | GTGCTTTT | |
| 43029794 | 1 | CGCCCAGCCTATCCA | 465 | GGG | CGCCCAGCCTATCCACCGGCGTTTTAGAGCTAGAAATAGCAAGTTA | 722 |
| | | CCGGC | | | AAATAAGGCTAGTCCGTTATCAACTTGAAAAAGTGGCACCGAGTCG | |
| | | | | | GTGCTTTT | |
| 43029795 | 1 | GCCCAGCCTATCCAC | 466 | GGG | GCCCAGCCTATCCACCGGCGGTTTTAGAGCTAGAAATAGCAAGTTA | 723 |
| | | CGGCG | | | AAATAAGGCTAGTCCGTTATCAACTTGAAAAAGTGGCACCGAGTCG | |
| | | | | | GTGCTTTT | |
| 43029795 | −1 | GTTAATCGGTCCCCC | 467 | TGG | GTTAATCGGTCCCCCGCCGGGTTTTAGAGCTAGAAATAGCAAGTTA | 724 |
| | | GCCGG | | | AAATAAGGCTAGTCCGTTATCAACTTGAAAAAGTGGCACCGAGTCG | |
| | | | | | GTGCTTTT | |
| 43029796 | 1 | CCCAGCCTATCCACC | 468 | GGG | CCCAGCCTATCCACCGGCGGGTTTTAGAGCTAGAAATAGCAAGTTA | 725 |
| | | GGCGG | | | AAATAAGGCTAGTCCGTTATCAACTTGAAAAAGTGGCACCGAGTCG | |
| | | | | | GTGCTTTT | |
| 43029798 | −1 | ATGGTTAATCGGTCC | 2513 | CGG | ATGGTTAATCGGTCCCCCGCGTTTTAGAGCTAGAAATAGCAAGTTA | 726 |
| | | CCCGC | | | AAATAAGGCTAGTCCGTTATCAACTTGAAAAAGTGGCACCGAGTCG | |
| | | | | | GTGCTTTT | |
| 43029809 | −1 | GGTGGGGGTTAATGG | 469 | CGG | GGTGGGGGTTAATGGTTAATGTTTTAGAGCTAGAAATAGCAAGTTA | 727 |
| | | TTAAT | | | AAATAAGGCTAGTCCGTTATCAACTTGAAAAAGTGGCACCGAGTCG | |
| | | | | | GTGCTTTT | |
| 43029817 | −1 | CGGGGAGGGGTGGGG | 470 | TGG | CGGGGAGGGGTGGGGGTTAAGTTTTAGAGCTAGAAATAGCAAGTTA | 728 |
| | | GTTAA | | | AAATAAGGCTAGTCCGTTATCAACTTGAAAAAGTGGCACCGAGTCG | |
| | | | | | GTGCTTTT | |
| 43029824 | −1 | GCTCTGCCGGGGAGG | 471 | GGG | GCTCTGCCGGGGAGGGGTGGGTTTTAGAGCTAGAAATAGCAAGTTA | 729 |
| | | GGTGG | | | AAATAAGGCTAGTCCGTTATCAACTTGAAAAAGTGGCACCGAGTCG | |
| | | | | | GTGCTTTT | |
| 43029825 | −1 | GGCTCTGCCGGGGAG | 472 | GGG | GGCTCTGCCGGGGAGGGGTGGTTTTAGAGCTAGAAATAGCAAGTTA | 730 |
| | | GGGTG | | | AAATAAGGCTAGTCCGTTATCAACTTGAAAAAGTGGCACCGAGTCG | |
| | | | | | GTGCTTTT | |
| 43029826 | −1 | AGGCTCTGCCGGGGA | 473 | GGG | AGGCTCTGCCGGGGAGGGGTGTTTTAGAGCTAGAAATAGCAAGTTA | 731 |
| | | GGGGT | | | AAATAAGGCTAGTCCGTTATCAACTTGAAAAAGTGGCACCGAGTCG | |
| | | | | | GTGCTTTT | |
| 43029827 | −1 | GAGGCTCTGCCGGGG | 474 | TGG | GAGGCTCTGCCGGGGAGGGGGTTTTAGAGCTAGAAATAGCAAGTTA | 732 |
| | | AGGGG | | | AAATAAGGCTAGTCCGTTATCAACTTGAAAAAGTGGCACCGAGTCG | |
| | | | | | GTGCTTTT | |
| 43029829 | 1 | CATTAACCCCCACCC | 475 | CGG | CATTAACCCCCACCCCTCCCGTTTTAGAGCTAGAAATAGCAAGTTA | 733 |
| | | CTCCC | | | AAATAAGGCTAGTCCGTTATCAACTTGAAAAAGTGGCACCGAGTCG | |
| | | | | | GTGCTTTT | |
| 43029830 | −1 | GTGGAGGCTCTGCCG | 476 | GGG | GTGGAGGCTCTGCCGGGGAGGTTTTAGAGCTAGAAATAGCAAGTTA | 734 |
| | | GGGAG | | | AAATAAGGCTAGTCCGTTATCAACTTGAAAAAGTGGCACCGAGTCG | |
| | | | | | GTGCTTTT | |
| 43029831 | −1 | GGTGGAGGCTCTGCC | 477 | GGG | GGTGGAGGCTCTGCCGGGGAGTTTTAGAGCTAGAAATAGCAAGTTA | 735 |
| | | GGGGA | | | AAATAAGGCTAGTCCGTTATCAACTTGAAAAAGTGGCACCGAGTCG | |
| | | | | | GTGCTTTT | |
| 43029832 | −1 | GGGTGGAGGCTCTGC | 478 | AGG | GGGTGGAGGCTCTGCCGGGGGTTTTAGAGCTAGAAATAGCAAGTTA | 736 |
| | | CGGGG | | | AAATAAGGCTAGTCCGTTATCAACTTGAAAAAGTGGCACCGAGTCG | |
| | | | | | GTGCTTTT | |
| 43029835 | −1 | AAGGGGTGGAGGCTC | 479 | GGG | AAGGGGTGGAGGCTCTGCCGGTTTTAGAGCTAGAAATAGCAAGTTA | 737 |
| | | TGCCG | | | AAATAAGGCTAGTCCGTTATCAACTTGAAAAAGTGGCACCGAGTCG | |
| | | | | | GTGCTTTT | |
| 43029836 | −1 | GAAGGGGTGGAGGCT | 480 | GGG | GAAGGGGTGGAGGCTCTGCCGTTTTAGAGCTAGAAATAGCAAGTTA | 738 |
| | | CTGCC | | | AAATAAGGCTAGTCCGTTATCAACTTGAAAAAGTGGCACCGAGTCG | |
| | | | | | GTGCTTTT | |
| 43029837 | −1 | TGAAGGGGTGGAGGC | 481 | CGG | TGAAGGGGTGGAGGCTCTGCGTTTTAGAGCTAGAAATAGCAAGTTA | 739 |
| | | TCTGC | | | AAATAAGGCTAGTCCGTTATCAACTTGAAAAAGTGGCACCGAGTCG | |
| | | | | | GTGCTTTT | |
| 43029846 | −1 | TAGCCTCTGTGAAGG | 482 | AGG | TAGCCTCTGTGAAGGGGTGGGTTTTAGAGCTAGAAATAGCAAGTTA | 740 |
| | | GGTGG | | | AAATAAGGCTAGTCCGTTATCAACTTGAAAAAGTGGCACCGAGTCG | |
| | | | | | GTGCTTTT | |
| 43029849 | −1 | GCCTAGCCTCTGTGA | 483 | TGG | GCCTAGCCTCTGTGAAGGGGGTTTTAGAGCTAGAAATAGCAAGTTA | 741 |
| | | AGGGG | | | AAATAAGGCTAGTCCGTTATCAACTTGAAAAAGTGGCACCGAGTCG | |
| | | | | | GTGCTTTT | |
| 43029852 | −1 | TTGGCCTAGCCTCTG | 484 | GGG | TTGGCCTAGCCTCTGTGAAGGTTTTAGAGCTAGAAATAGCAAGTTA | 742 |
| | | TGAAG | | | AAATAAGGCTAGTCCGTTATCAACTTGAAAAAGTGGCACCGAGTCG | |
| | | | | | GTGCTTTT | |
| 43029853 | −1 | CTTGGCCTAGCCTCT | 485 | GGG | CTTGGCCTAGCCTCTGTGAAGTTTTAGAGCTAGAAATAGCAAGTTA | 743 |
| | | GTGAA | | | AAATAAGGCTAGTCCGTTATCAACTTGAAAAAGTGGCACCGAGTCG | |
| | | | | | GTGCTTTT | |
| 43029854 | 1 | GAGCCTCCACCCCTT | 486 | AGG | GAGCCTCCACCCCTTCACAGGTTTTAGAGCTAGAAATAGCAAGTTA | 744 |
| | | CACAG | | | AAATAAGGCTAGTCCGTTATCAACTTGAAAAAGTGGCACCGAGTCG | |
| | | | | | GTGCTTTT | |
| 43029854 | −1 | TCTTGGCCTAGCCTC | 487 | AGG | TCTTGGCCTAGCCTCTGTGAGTTTTAGAGCTAGAAATAGCAAGTTA | 745 |
| | | TGTGA | | | AAATAAGGCTAGTCCGTTATCAACTTGAAAAAGTGGCACCGAGTCG | |
| | | | | | GTGCTTTT | |
| 43029859 | 1 | TCCACCCCTTCACAG | 488 | AGG | TCCACCCCTTCACAGAGGCTGTTTTAGAGCTAGAAATAGCAAGTTA | 746 |
| | | AGGCT | | | AAATAAGGCTAGTCCGTTATCAACTTGAAAAAGTGGCACCGAGTCG | |
| | | | | | GTGCTTTT | |
| 43029871 | −1 | GGAAGATCTGCTGGG | 489 | TGG | GGAAGATCTGCTGGGAGTCTGTTTTAGAGCTAGAAATAGCAAGTTA | 747 |
| | | AGTCT | | | AAATAAGGCTAGTCCGTTATCAACTTGAAAAAGTGGCACCGAGTCG | |
| | | | | | GTGCTTTT | |
| 43029879 | −1 | GTCCTCTGGGAAGAT | 2514 | GGG | GTCCTCTGGGAAGATCTGCTGTTTTAGAGCTAGAAATAGCAAGTTA | 748 |
| | | CTGCT | | | AAATAAGGCTAGTCCGTTATCAACTTGAAAAAGTGGCACCGAGTCG | |
| | | | | | GTGCTTTT | |
| 43029880 | −1 | CGTCCTCTGGGAAGA | 490 | TGG | CGTCCTCTGGGAAGATCTGCGTTTTAGAGCTAGAAATAGCAAGTTA | 749 |
| | | TCTGC | | | AAATAAGGCTAGTCCGTTATCAACTTGAAAAAGTGGCACCGAGTCG | |
| | | | | | GTGCTTTT | |
| 43029888 | 1 | CTCCCAGCAGATCTT | 491 | AGG | CTCCCAGCAGATCTTCCCAGGTTTTAGAGCTAGAAATAGCAAGTTA | 750 |
| | | CCCAG | | | AAATAAGGCTAGTCCGTTATCAACTTGAAAAAGTGGCACCGAGTCG | |
| | | | | | GTGCTTTT | |
| 43029892 | 1 | CAGCAGATCTTCCCA | 492 | CGG | CAGCAGATCTTCCCAGAGGAGTTTTAGAGCTAGAAATAGCAAGTTA | 751 |
| | | GAGGA | | | AAATAAGGCTAGTCCGTTATCAACTTGAAAAAGTGGCACCGAGTCG | |
| | | | | | GTGCTTTT | |
| 43029892 | −1 | TTCCTTTCAAACCGT | 493 | GGG | TTCCTTTCAAACCGTCCTCTGTTTTAGAGCTAGAAATAGCAAGTTA | 752 |
| | | CCTCT | | | AAATAAGGCTAGTCCGTTATCAACTTGAAAAAGTGGCACCGAGTCG | |
| | | | | | GTGCTTTT | |
| 43029893 | −1 | CTTCCTTTCAAACCG | 494 | TGG | CTTCCTTTCAAACCGTCCTCGTTTTAGAGCTAGAAATAGCAAGTTA | 753 |
| | | TCCTC | | | AAATAAGGCTAGTCCGTTATCAACTTGAAAAAGTGGCACCGAGTCG | |
| | | | | | GTGCTTTT | |
| 43029901 | 1 | TTCCCAGAGGACGGT | 495 | AGG | TTCCCAGAGGACGGTTTGAAGTTTTAGAGCTAGAAATAGCAAGTTA | 754 |
| | | TTGAA | | | AAATAAGGCTAGTCCGTTATCAACTTGAAAAAGTGGCACCGAGTCG | |
| | | | | | GTGCTTTT | |
| 43029905 | 1 | CAGAGGACGGTTTGA | 496 | AGG | CAGAGGACGGTTTGAAAGGAGTTTTAGAGCTAGAAATAGCAAGTTA | 755 |
| | | AAGGA | | | AAATAAGGCTAGTCCGTTATCAACTTGAAAAAGTGGCACCGAGTCG | |
| | | | | | GTGCTTTT | |
| 43029913 | 1 | GGTTTGAAAGGAAGG | 2515 | AGG | GGTTTGAAAGGAAGGCAGAGGTTTTAGAGCTAGAAATAGCAAGTTA | 756 |
| | | CAGAG | | | AAATAAGGCTAGTCCGTTATCAACTTGAAAAAGTGGCACCGAGTCG | |
| | | | | | GTGCTTTT | |
| 43029914 | 1 | GTTTGAAAGGAAGGC | 497 | GGG | GTTTGAAAGGAAGGCAGAGAGTTTTAGAGCTAGAAATAGCAAGTTA | 757 |
| | | AGAGA | | | AAATAAGGCTAGTCCGTTATCAACTTGAAAAAGTGGCACCGAGTCG | |
| | | | | | GTGCTTTT | |
| 43029920 | 1 | AAGGAAGGCAGAGAG | 498 | TGG | AAGGAAGGCAGAGAGGGCACGTTTTAGAGCTAGAAATAGCAAGTTA | 758 |
| | | GGCAC | | | AAATAAGGCTAGTCCGTTATCAACTTGAAAAAGTGGCACCGAGTCG | |
| | | | | | GTGCTTTT | |
| 43029921 | 1 | AGGAAGGCAGAGAGG | 499 | GGG | AGGAAGGCAGAGAGGGCACTGTTTTAGAGCTAGAAATAGCAAGTTA | 759 |
| | | GCACT | | | AAATAAGGCTAGTCCGTTATCAACTTGAAAAAGTGGCACCGAGTCG | |
| | | | | | GTGCTTTT | |
| 43029924 | 1 | AAGGCAGAGAGGGCA | 500 | AGG | AAGGCAGAGAGGGCACTGGGGTTTTAGAGCTAGAAATAGCAAGTTA | 760 |
| | | CTGGG | | | AAATAAGGCTAGTCCGTTATCAACTTGAAAAAGTGGCACCGAGTCG | |
| | | | | | GTGCTTTT | |
| 43029927 | 1 | GCAGAGAGGGCACTG | 501 | AGG | GCAGAGAGGGCACTGGGAGGGTTTTAGAGCTAGAAATAGCAAGTTA | 761 |
| | | GGAGG | | | AAATAAGGCTAGTCCGTTATCAACTTGAAAAAGTGGCACCGAGTCG | |
| | | | | | GTGCTTTT | |
| 43029933 | 1 | AGGGCACTGGGAGGA | 502 | TGG | AGGGCACTGGGAGGAGGCAGGTTTTAGAGCTAGAAATAGCAAGTTA | 762 |
| | | GGCAG | | | AAATAAGGCTAGTCCGTTATCAACTTGAAAAAGTGGCACCGAGTCG | |
| | | | | | GTGCTTTT | |
| 43029934 | 1 | GGGCACTGGGAGGAGGCAGT | 503 | GGG | GGGCACTGGGAGGAGGCAGTGTTTTAGAGCTAGAAATAGCAAGTTA | 763 |
| | | | | | AAATAAGGCTAGTCCGTTATCAACTTGAAAAAGTGGCACCGAGTCG | |
| | | | | | GTGCTTTT | |
| 43029937 | 1 | CACTGGGAGGAGGCA | 504 | AGG | CACTGGGAGGAGGCAGTGGGGTTTTAGAGCTAGAAATAGCAAGTTA | 764 |
| | | GTGGG | | | AAATAAGGCTAGTCCGTTATCAACTTGAAAAAGTGGCACCGAGTCG | |
| | | | | | GTGCTTTT | |
| 43029938 | 1 | ACTGGGAGGAGGCAG | 505 | GGG | ACTGGGAGGAGGCAGTGGGAGTTTTAGAGCTAGAAATAGCAAGTTA | 765 |
| | | TGGGA | | | AAATAAGGCTAGTCCGTTATCAACTTGAAAAAGTGGCACCGAGTCG | |
| | | | | | GTGCTTTT | |
| 43029941 | 1 | GGGAGGAGGCAGTGG | 506 | CGG | GGGAGGAGGCAGTGGGAGGGGTTTTAGAGCTAGAAATAGCAAGTTA | 766 |
| | | GAGGG | | | AAATAAGGCTAGTCCGTTATCAACTTGAAAAAGTGGCACCGAGTCG | |
| | | | | | GTGCTTTT | |
| 43029944 | 1 | AGGAGGCAGTGGGAG | 507 | AGG | AGGAGGCAGTGGGAGGGCGGGTTTTAGAGCTAGAAATAGCAAGTTA | 767 |
| | | GGCGG | | | AAATAAGGCTAGTCCGTTATCAACTTGAAAAAGTGGCACCGAGTCG | |
| | | | | | GTGCTTTT | |
| 43029945 | 1 | GGAGGCAGTGGGAGG | 508 | GGG | GGAGGCAGTGGGAGGGCGGAGTTTTAGAGCTAGAAATAGCAAGTTA | 768 |
| | | GCGGA | | | AAATAAGGCTAGTCCGTTATCAACTTGAAAAAGTGGCACCGAGTCG | |
| | | | | | GTGCTTTT | |
| 43029948 | 1 | GGCAGTGGGAGGGCG | 509 | CGG | GGCAGTGGGAGGGCGGAGGGGTTTTAGAGCTAGAAATAGCAAGTTA | 769 |
| | | GAGGG | | | AAATAAGGCTAGTCCGTTATCAACTTGAAAAAGTGGCACCGAGTCG | |
| | | | | | GTGCTTTT | |
| 43029949 | 1 | GCAGTGGGAGGGCGG | 510 | GGG | GCAGTGGGAGGGCGGAGGGCGTTTTAGAGCTAGAAATAGCAAGTTA | 770 |
| | | AGGGC | | | AAATAAGGCTAGTCCGTTATCAACTTGAAAAAGTGGCACCGAGTCG | |
| | | | | | GTGCTTTT | |
| 43029950 | 1 | CAGTGGGAGGGCGGA | 511 | GGG | CAGTGGGAGGGCGGAGGGCGGTTTTAGAGCTAGAAATAGCAAGTTA | 771 |
| | | GGGCG | | | AAATAAGGCTAGTCCGTTATCAACTTGAAAAAGTGGCACCGAGTCG | |
| | | | | | GTGCTTTT | |
| 43029951 | 1 | AGTGGGAGGGCGGAG | 512 | GGG | AGTGGGAGGGCGGAGGGCGGGTTTTAGAGCTAGAAATAGCAAGTTA | 772 |
| | | GGCGG | | | AAATAAGGCTAGTCCGTTATCAACTTGAAAAAGTGGCACCGAGTCG | |
| | | | | | GTGCTTTT | |
| 43029958 | 1 | GGGCGGAGGGCGGGG | 513 | CGG | GGGCGGAGGGCGGGGGCCTTGTTTTAGAGCTAGAAATAGCAAGTTA | 773 |
| | | GCCTT | | | AAATAAGGCTAGTCCGTTATCAACTTGAAAAAGTGGCACCGAGTCG | |
| | | | | | GTGCTTTT | |
| 43029959 | 1 | GGCGGAGGGCGGGGG | 514 | GGG | GGCGGAGGGCGGGGGCCTTCGTTTTAGAGCTAGAAATAGCAAGTTA | 774 |
| | | CCTTC | | | AAATAAGGCTAGTCCGTTATCAACTTGAAAAAGTGGCACCGAGTCG | |
| | | | | | GTGCTTTT | |
| 43029960 | 1 | GCGGAGGGCGGGGGC | 515 | GGG | GCGGAGGGCGGGGGCCTTCGGTTTTAGAGCTAGAAATAGCAAGTTA | 775 |
| | | CTTCG | | | AAATAAGGCTAGTCCGTTATCAACTTGAAAAAGTGGCACCGAGTCG | |
| | | | | | GTGCTTTT | |
| 43029963 | 1 | GAGGGCGGGGGCCTT | 516 | TGG | GAGGGCGGGGGCCTTCGGGGGTTTTAGAGCTAGAAATAGCAAGTTA | 776 |
| | | CGGGG | | | AAATAAGGCTAGTCCGTTATCAACTTGAAAAAGTGGCACCGAGTCG | |
| | | | | | GTGCTTTT | |
| 43029963 | −1 | ACCCTGGGCGCCCAC | 2516 | AGG | ACCCTGGGCGCCCACCCCGAGTTTTAGAGCTAGAAATAGCAAGTTA | 777 |
| | | CCCGA | | | AAATAAGGCTAGTCCGTTATCAACTTGAAAAAGTGGCACCGAGTCG | |
| | | | | | GTGCTTTT | |
| 43029964 | 1 | AGGGCGGGGGCCTTC | 517 | GGG | AGGGCGGGGGCCTTCGGGGTGTTTTAGAGCTAGAAATAGCAAGTTA | 778 |
| | | GGGGT | | | AAATAAGGCTAGTCCGTTATCAACTTGAAAAAGTGGCACCGAGTCG | |
| | | | | | GTGCTTTT | |
| 43029972 | 1 | GGCCTTCGGGGTGGG | 518 | AGG | GGCCTTCGGGGTGGGCGCCCGTTTTAGAGCTAGAAATAGCAAGTTA | 779 |
| | | CGCCC | | | AAATAAGGCTAGTCCGTTATCAACTTGAAAAAGTGGCACCGAGTCG | |
| | | | | | GTGCTTTT | |
| 43029973 | 1 | GCCTTCGGGGTGGGC | 519 | GGG | GCCTTCGGGGTGGGCGCCCAGTTTTAGAGCTAGAAATAGCAAGTTA | 780 |
| | | GCCCA | | | AAATAAGGCTAGTCCGTTATCAACTTGAAAAAGTGGCACCGAGTCG | |
| | | | | | GTGCTTTT | |
| 43029977 | 1 | TCGGGGTGGGCGCCC | 2517 | AGG | TCGGGGTGGGCGCCCAGGGTGTTTTAGAGCTAGAAATAGCAAGTTA | 781 |
| | | AGGGT | | | AAATAAGGCTAGTCCGTTATCAACTTGAAAAAGTGGCACCGAGTCG | |
| | | | | | GTGCTTTT | |
| 43029978 | 1 | CGGGGTGGGCGCCCA | 520 | GGG | CGGGGTGGGCGCCCAGGGTAGTTTTAGAGCTAGAAATAGCAAGTTA | 782 |
| | | GGGTA | | | AAATAAGGCTAGTCCGTTATCAACTTGAAAAAGTGGCACCGAGTCG | |
| | | | | | GTGCTTTT | |
| 43029978 | −1 | GCGGCCACCTGCCCT | 521 | GGG | GCGGCCACCTGCCCTACCCTGTTTTAGAGCTAGAAATAGCAAGTTA | 783 |
| | | ACCCT | | | AAATAAGGCTAGTCCGTTATCAACTTGAAAAAGTGGCACCGAGTCG | |
| | | | | | GTGCTTTT | |
| 43029979 | −1 | CGCGGCCACCTGCCC | 522 | TGG | CGCGGCCACCTGCCCTACCCGTTTTAGAGCTAGAAATAGCAAGTTA | 784 |
| | | TACCC | | | AAATAAGGCTAGTCCGTTATCAACTTGAAAAAGTGGCACCGAGTCG | |
| | | | | | GTGCTTTT | |
| 43029982 | 1 | GTGGGCGCCCAGGGT | 523 | AGG | GTGGGCGCCCAGGGTAGGGCGTTTTAGAGCTAGAAATAGCAAGTTA | 785 |
| | | AGGGC | | | AAATAAGGCTAGTCCGTTATCAACTTGAAAAAGTGGCACCGAGTCG | |
| | | | | | GTGCTTTT | |
| 43029985 | 1 | GGCGCCCAGGGTAGG | 524 | TGG | GGCGCCCAGGGTAGGGCAGGGTTTTAGAGCTAGAAATAGCAAGTTA | 786 |
| | | GCAGG | | | AAATAAGGCTAGTCCGTTATCAACTTGAAAAAGTGGCACCGAGTCG | |
| | | | | | GTGCTTTT | |
| 43029991 | 1 | CAGGGTAGGGCAGGT | 525 | CGG | CAGGGTAGGGCAGGTGGCCGGTTTTAGAGCTAGAAATAGCAAGTTA | 787 |
| | | GGCCG | | | AAATAAGGCTAGTCCGTTATCAACTTGAAAAAGTGGCACCGAGTCG | |
| | | | | | GTGCTTTT | |
| 43029996 | 1 | TAGGGCAGGTGGCCG | 526 | TGG | TAGGGCAGGTGGCCGCGGCGGTTTTAGAGCTAGAAATAGCAAGTTA | 788 |
| | | CGGCG | | | AAATAAGGCTAGTCCGTTATCAACTTGAAAAAGTGGCACCGAGTCG | |
| | | | | | GTGCTTTT | |
| 43029997 | −1 | TTCTCCCTGCCTCCA | 2518 | CGG | TTCTCCCTGCCTCCACGCCGGTTTTAGAGCTAGAAATAGCAAGTTA | 789 |
| | | CGCCG | | | AAATAAGGCTAGTCCGTTATCAACTTGAAAAAGTGGCACCGAGTCG | |
| | | | | | GTGCTTTT | |
| 43029999 | 1 | GGCAGGTGGCCGCGG | 527 | AGG | GGCAGGTGGCCGCGGCGTGGGTTTTAGAGCTAGAAATAGCAAGTTA | 790 |
| | | CGTGG | | | AAATAAGGCTAGTCCGTTATCAACTTGAAAAAGTGGCACCGAGTCG | |
| | | | | | GTGCTTTT | |
| 43030003 | 1 | GGTGGCCGCGGCGTG | 528 | AGG | GGTGGCCGCGGCGTGGAGGCGTTTTAGAGCTAGAAATAGCAAGTTA | 791 |
| | | GAGGC | | | AAATAAGGCTAGTCCGTTATCAACTTGAAAAAGTGGCACCGAGTCG | |
| | | | | | GTGCTTTT | |
| 43030004 | 1 | GTGGCCGCGGCGTGG | 529 | GGG | GTGGCCGCGGCGTGGAGGCAGTTTTAGAGCTAGAAATAGCAAGTTA | 792 |
| | | AGGCA | | | AAATAAGGCTAGTCCGTTATCAACTTGAAAAAGTGGCACCGAGTCG | |
| | | | | | GTGCTTTT | |
| 43030029 | −1 | GTCCATGTCGACGAG | 530 | TGG | GTCCATGTCGACGAGGGTTTGTTTTAGAGCTAGAAATAGCAAGTTA | 793 |
| | | GGTTT | | | AAATAAGGCTAGTCCGTTATCAACTTGAAAAAGTGGCACCGAGTCG | |
| | | | | | GTGCTTTT | |
| 43030035 | −1 | GGCCATGTCCATGTC | 531 | GGG | GGCCATGTCCATGTCGACGAGTTTTAGAGCTAGAAATAGCAAGTTA | 794 |
| | | GACGA | | | AAATAAGGCTAGTCCGTTATCAACTTGAAAAAGTGGCACCGAGTCG | |
| | | | | | GTGCTTTT | |
| 43030036 | −1 | CGGCCATGTCCATGT | 532 | AGG | CGGCCATGTCCATGTCGACGGTTTTAGAGCTAGAAATAGCAAGTTA | 795 |
| | | CGACG | | | AAATAAGGCTAGTCCGTTATCAACTTGAAAAAGTGGCACCGAGTCG | |
| | | | | | GTGCTTTT | |
| 43030038 | 1 | CTCCAAAACCCTCGT | 533 | TGG | CTCCAAAACCCTCGTCGACAGTTTTAGAGCTAGAAATAGCAAGTTA | 796 |
| | | CGACA | | | AAATAAGGCTAGTCCGTTATCAACTTGAAAAAGTGGCACCGAGTCG | |
| | | | | | GTGCTTTT | |
| 43030044 | 1 | AACCCTCGTCGACAT | 534 | TGG | AACCCTCGTCGACATGGACAGTTTTAGAGCTAGAAATAGCAAGTTA | 797 |
| | | GGACA | | | AAATAAGGCTAGTCCGTTATCAACTTGAAAAAGTGGCACCGAGTCG | |
| | | | | | GTGCTTTT | |
| 43030056 | −1 | GTCCAGTGCAGCACT | 535 | CGG | GTCCAGTGCAGCACTGTAGTGTTTTAGAGCTAGAAATAGCAAGTTA | 798 |
| | | GTAGT | | | AAATAAGGCTAGTCCGTTATCAACTTGAAAAAGTGGCACCGAGTCG | |
| | | | | | GTGCTTTT | |
| 43030065 | 1 | GGCCGACTACAGTGC | 536 | TGG | GGCCGACTACAGTGCTGCACGTTTTAGAGCTAGAAATAGCAAGTTA | 799 |
| | | TGCAC | | | AAATAAGGCTAGTCCGTTATCAACTTGAAAAAGTGGCACCGAGTCG | |
| | | | | | GTGCTTTT | |
| 43030078 | −1 | ATTCCAGGGTGGTGT | 537 | GGG | ATTCCAGGGTGGTGTAGGCTGTTTTAGAGCTAGAAATAGCAAGTTA | 800 |
| | | AGGCT | | | AAATAAGGCTAGTCCGTTATCAACTTGAAAAAGTGGCACCGAGTCG | |
| | | | | | GTGCTTTT | |
| 43030079 | −1 | AATTCCAGGGTGGTG | 538 | TGG | AATTCCAGGGTGGTGTAGGCGTTTTAGAGCTAGAAATAGCAAGTTA | 801 |
| | | TAGGC | | | AAATAAGGCTAGTCCGTTATCAACTTGAAAAAGTGGCACCGAGTCG | |
| | | | | | GTGCTTTT | |
| 43030083 | −1 | CTCAAATTCCAGGGT | 539 | AGG | CTCAAATTCCAGGGTGGTGTGTTTTAGAGCTAGAAATAGCAAGTTA | 802 |
| | | GGTGT | | | AAATAAGGCTAGTCCGTTATCAACTTGAAAAAGTGGCACCGAGTCG | |
| | | | | | GTGCTTTT | |
| 43030086 | 1 | GGACCCAGCCTACACCACCC | 540 | TGG | GGACCCAGCCTACACCACCCGTTTTAGAGCTAGAAATAGCAAGTTA | 803 |
| | | | | | AAATAAGGCTAGTCCGTTATCAACTTGAAAAAGTGGCACCGAGTCG | |
| | | | | | GTGCTTTT | |
| 43030089 | −1 | CACATTCTCAAATTC | 541 | TGG | CACATTCTCAAATTCCAGGGGTTTTAGAGCTAGAAATAGCAAGTTA | 804 |
| | | CAGGG | | | AAATAAGGCTAGTCCGTTATCAACTTGAAAAAGTGGCACCGAGTCG | |
| | | | | | GTGCTTTT | |
| 43030092 | −1 | CTGCACATTCTCAAA | 542 | GGG | CTGCACATTCTCAAATTCCAGTTTTAGAGCTAGAAATAGCAAGTTA | 805 |
| | | TTCCA | | | AAATAAGGCTAGTCCGTTATCAACTTGAAAAAGTGGCACCGAGTCG | |
| | | | | | GTGCTTTT | |
| 43030093 | −1 | CCTGCACATTCTCAA | 543 | AGG | CCTGCACATTCTCAAATTCCGTTTTAGAGCTAGAAATAGCAAGTTA | 806 |
| | | ATTCC | | | AAATAAGGCTAGTCCGTTATCAACTTGAAAAAGTGGCACCGAGTCG | |
| | | | | | GTGCTTTT | |
| 43030104 | 1 | CCTGGAATTTGAGAA | 544 | AGG | CCTGGAATTTGAGAATGTGCGTTTTAGAGCTAGAAATAGCAAGTTA | 807 |
| | | TGTGC | | | AAATAAGGCTAGTCCGTTATCAACTTGAAAAAGTGGCACCGAGTCG | |
| | | | | | GTGCTTTT | |
| 43030116 | 1 | GAATGTGCAGGTGTT | 545 | TGG | GAATGTGCAGGTGTTGACGAGTTTTAGAGCTAGAAATAGCAAGTTA | 808 |
| | | GACGA | | | AAATAAGGCTAGTCCGTTATCAACTTGAAAAAGTGGCACCGAGTCG | |
| | | | | | GTGCTTTT | |
| 43030117 | 1 | AATGTGCAGGTGTTG | 546 | GGG | AATGTGCAGGTGTTGACGATGTTTTAGAGCTAGAAATAGCAAGTTA | 809 |
| | | ACGAT | | | AAATAAGGCTAGTCCGTTATCAACTTGAAAAAGTGGCACCGAGTCG | |
| | | | | | GTGCTTTT | |
| 43030123 | 1 | CAGGTGTTGACGATG | 547 | TGG | CAGGTGTTGACGATGGGCAAGTTTTAGAGCTAGAAATAGCAAGTTA | 810 |
| | | GGCAA | | | AAATAAGGCTAGTCCGTTATCAACTTGAAAAAGTGGCACCGAGTCG | |
| | | | | | GTGCTTTT | |
| 43030127 | 1 | TGTTGACGATGGGCA | 548 | AGG | TGTTGACGATGGGCAATGGTGTTTTAGAGCTAGAAATAGCAAGTTA | 811 |
| | | ATGGT | | | AAATAAGGCTAGTCCGTTATCAACTTGAAAAAGTGGCACCGAGTCG | |
| | | | | | GTGCTTTT | |
| 43030130 | 1 | TGACGATGGGCAATG | 549 | TGG | TGACGATGGGCAATGGTAGGGTTTTAGAGCTAGAAATAGCAAGTTA | 812 |
| | | GTAGG | | | AAATAAGGCTAGTCCGTTATCAACTTGAAAAAGTGGCACCGAGTCG | |
| | | | | | GTGCTTTT | |
| 43030131 | 1 | GACGATGGGCAATGGTAGGT | 550 | GGG | GACGATGGGCAATGGTAGGTGTTTTAGAGCTAGAAATAGCAAGTTA | 813 |
| | | | | | AAATAAGGCTAGTCCGTTATCAACTTGAAAAAGTGGCACCGAGTCG | |
| | | | | | GTGCTTTT | |
| 43030132 | 1 | ACGATGGGCAATGGT | 551 | GGG | ACGATGGGCAATGGTAGGTGGTTTTAGAGCTAGAAATAGCAAGTTA | 814 |
| | | AGGTG | | | AAATAAGGCTAGTCCGTTATCAACTTGAAAAAGTGGCACCGAGTCG | |
| | | | | | GTGCTTTT | |
| 43030133 | 1 | CGATGGGCAATGGTA | 552 | GGG | CGATGGGCAATGGTAGGTGGGTTTTAGAGCTAGAAATAGCAAGTTA | 815 |
| | | GGTGG | | | AAATAAGGCTAGTCCGTTATCAACTTGAAAAAGTGGCACCGAGTCG | |
| | | | | | GTGCTTTT | |
| 43030147 | 1 | AGGTGGGGGCAGATG | 553 | AGG | AGGTGGGGGCAGATGTGCCCGTTTTAGAGCTAGAAATAGCAAGTTA | 816 |
| | | TGCCC | | | AAATAAGGCTAGTCCGTTATCAACTTGAAAAAGTGGCACCGAGTCG | |
| | | | | | GTGCTTTT | |
| 43030153 | −1 | CTGCCCCCACTGGCA | 554 | GGG | CTGCCCCCACTGGCACACCTGTTTTAGAGCTAGAAATAGCAAGTTA | 817 |
| | | CACCT | | | AAATAAGGCTAGTCCGTTATCAACTTGAAAAAGTGGCACCGAGTCG | |
| | | | | | GTGCTTTT | |
| 43030154 | −1 | CCTGCCCCCACTGGC | 555 | TGG | CCTGCCCCCACTGGCACACCGTTTTAGAGCTAGAAATAGCAAGTTA | 818 |
| | | ACACC | | | AAATAAGGCTAGTCCGTTATCAACTTGAAAAAGTGGCACCGAGTCG | |
| | | | | | GTGCTTTT | |
| 43030158 | 1 | GATGTGCCCAGGTGT | 556 | TGG | GATGTGCCCAGGTGTGCCAGGTTTTAGAGCTAGAAATAGCAAGTTA | 819 |
| | | GCCAG | | | AAATAAGGCTAGTCCGTTATCAACTTGAAAAAGTGGCACCGAGTCG | |
| | | | | | GTGCTTTT | |
| 43030159 | 1 | ATGTGCCCAGGTGTG | 557 | GGG | ATGTGCCCAGGTGTGCCAGTGTTTTAGAGCTAGAAATAGCAAGTTA | 820 |
| | | CCAGT | | | AAATAAGGCTAGTCCGTTATCAACTTGAAAAAGTGGCACCGAGTCG | |
| | | | | | GTGCTTTT | |
| 43030160 | 1 | TGTGCCCAGGTGTGC | 558 | GGG | TGTGCCCAGGTGTGCCAGTGGTTTTAGAGCTAGAAATAGCAAGTTA | 821 |
| | | CAGTG | | | AAATAAGGCTAGTCCGTTATCAACTTGAAAAAGTGGCACCGAGTCG | |
| | | | | | GTGCTTTT | |
| 43030161 | 1 | GTGCCCAGGTGTGCC | 559 | GGG | GTGCCCAGGTGTGCCAGTGGGTTTTAGAGCTAGAAATAGCAAGTTA | 822 |
| | | AGTGG | | | AAATAAGGCTAGTCCGTTATCAACTTGAAAAAGTGGCACCGAGTCG | |
| | | | | | GTGCTTTT | |
| 43030163 | −1 | CCAGGCACACCTGCC | 560 | TGG | CCAGGCACACCTGCCCCCACGTTTTAGAGCTAGAAATAGCAAGTTA | 823 |
| | | CCCAC | | | AAATAAGGCTAGTCCGTTATCAACTTGAAAAAGTGGCACCGAGTCG | |
| | | | | | GTGCTTTT | |
| 43030165 | 1 | CCAGGTGTGCCAGTG | 561 | AGG | CCAGGTGTGCCAGTGGGGGCGTTTTAGAGCTAGAAATAGCAAGTTA | 824 |
| | | GGGGC | | | AAATAAGGCTAGTCCGTTATCAACTTGAAAAAGTGGCACCGAGTCG | |
| | | | | | GTGCTTTT | |
| 43030174 | 1 | CCAGTGGGGGCAGGT | 562 | TGG | CCAGTGGGGGCAGGTGTGCCGTTTTAGAGCTAGAAATAGCAAGTTA | 825 |
| | | GTGCC | | | AAATAAGGCTAGTCCGTTATCAACTTGAAAAAGTGGCACCGAGTCG | |
| | | | | | GTGCTTTT | |
| 43030175 | 1 | CAGTGGGGGCAGGTG | 563 | GGG | CAGTGGGGGCAGGTGTGCCTGTTTTAGAGCTAGAAATAGCAAGTTA | 826 |
| | | TGCCT | | | AAATAAGGCTAGTCCGTTATCAACTTGAAAAAGTGGCACCGAGTCG | |
| | | | | | GTGCTTTT | |
| 43030181 | 1 | GGGCAGGTGTGCCTG | 564 | AGG | GGGCAGGTGTGCCTGGGTCCGTTTTAGAGCTAGAAATAGCAAGTTA | 827 |
| | | GGTCC | | | AAATAAGGCTAGTCCGTTATCAACTTGAAAAAGTGGCACCGAGTCG | |
| | | | | | GTGCTTTT | |
| 43030181 | −1 | AAAGATCTGCTCCTG | 565 | AGG | AAAGATCTGCTCCTGGACCCGTTTTAGAGCTAGAAATAGCAAGTTA | 828 |
| | | GACCC | | | AAATAAGGCTAGTCCGTTATCAACTTGAAAAAGTGGCACCGAGTCG | |
| | | | | | GTGCTTTT | |
| 43030188 | −1 | GAGTGCCAAAGATCT | 566 | TGG | GAGTGCCAAAGATCTGCTCCGTTTTAGAGCTAGAAATAGCAAGTTA | 829 |
| | | GCTCC | | | AAATAAGGCTAGTCCGTTATCAACTTGAAAAAGTGGCACCGAGTCG | |
| | | | | | GTGCTTTT | |
| 43030194 | 1 | TGGGTCCAGGAGCAG | 567 | TGG | TGGGTCCAGGAGCAGATCTTGTTTTAGAGCTAGAAATAGCAAGTTA | 830 |
| | | ATCTT | | | AAATAAGGCTAGTCCGTTATCAACTTGAAAAAGTGGCACCGAGTCG | |
| | | | | | GTGCTTTT | |
| 43030207 | 1 | AGATCTTTGGCACTC | 568 | TGG | AGATCTTTGGCACTCAACTTGTTTTAGAGCTAGAAATAGCAAGTTA | 831 |
| | | AACTT | | | AAATAAGGCTAGTCCGTTATCAACTTGAAAAAGTGGCACCGAGTCG | |
| | | | | | GTGCTTTT | |
| 43030208 | 1 | GATCTTTGGCACTCA | 569 | GGG | GATCTTTGGCACTCAACTTTGTTTTAGAGCTAGAAATAGCAAGTTA | 832 |
| | | ACTTT | | | AAATAAGGCTAGTCCGTTATCAACTTGAAAAAGTGGCACCGAGTCG | |
| | | | | | GTGCTTTT | |
| 43030209 | 1 | ATCTTTGGCACTCAACTTTG | 570 | GGG | ATCTTTGGCACTCAACTTTGGTTTTAGAGCTAGAAATAGCAAGTTA | 833 |
| | | | | | AAATAAGGCTAGTCCGTTATCAACTTGAAAAAGTGGCACCGAGTCG | |
| | | | | | GTGCTTTT | |
| 43030212 | 1 | TTTGGCACTCAACTT | 571 | TGG | TTTGGCACTCAACTTTGGGGGTTTTAGAGCTAGAAATAGCAAGTTA | 834 |
| | | TGGGG | | | AAATAAGGCTAGTCCGTTATCAACTTGAAAAAGTGGCACCGAGTCG | |
| | | | | | GTGCTTTT | |
| 43030213 | 1 | TTGGCACTCAACTTT | 572 | GGG | TTGGCACTCAACTTTGGGGTGTTTTAGAGCTAGAAATAGCAAGTTA | 835 |
| | | GGGGT | | | AAATAAGGCTAGTCCGTTATCAACTTGAAAAAGTGGCACCGAGTCG | |
| | | | | | GTGCTTTT | |
| 43030216 | 1 | GCACTCAACTTTGGG | 573 | AGG | GCACTCAACTTTGGGGTGGGGTTTTAGAGCTAGAAATAGCAAGTTA | 836 |
| | | GTGGG | | | AAATAAGGCTAGTCCGTTATCAACTTGAAAAAGTGGCACCGAGTCG | |
| | | | | | GTGCTTTT | |
| 43030233 | 1 | GGGAGGAGAATGATA | 574 | TGG | GGGAGGAGAATGATACAAAAGTTTTAGAGCTAGAAATAGCAAGTTA | 837 |
| | | CAAAA | | | AAATAAGGCTAGTCCGTTATCAACTTGAAAAAGTGGCACCGAGTCG | |
| | | | | | GTGCTTTT | |
| 43030237 | 1 | GGAGAATGATACAAA | 575 | AGG | GGAGAATGATACAAAATGGTGTTTTAGAGCTAGAAATAGCAAGTTA | 838 |
| | | ATGGT | | | AAATAAGGCTAGTCCGTTATCAACTTGAAAAAGTGGCACCGAGTCG | |
| | | | | | GTGCTTTT | |
| 43030241 | 1 | AATGATACAAAATGG | 576 | TGG | AATGATACAAAATGGTAGGTGTTTTAGAGCTAGAAATAGCAAGTTA | 839 |
| | | TAGGT | | | AAATAAGGCTAGTCCGTTATCAACTTGAAAAAGTGGCACCGAGTCG | |
| | | | | | GTGCTTTT | |
| 43030250 | 1 | AAATGGTAGGTTGGT | 577 | AGG | AAATGGTAGGTTGGTCCTACGTTTTAGAGCTAGAAATAGCAAGTTA | 840 |
| | | CCTAC | | | AAATAAGGCTAGTCCGTTATCAACTTGAAAAAGTGGCACCGAGTCG | |
| | | | | | GTGCTTTT | |
| 43030254 | −1 | CAACACCTGTGCTGG | 578 | AGG | CAACACCTGTGCTGGCCTGTGTTTTAGAGCTAGAAATAGCAAGTTA | 841 |
| | | CCTGT | | | AAATAAGGCTAGTCCGTTATCAACTTGAAAAAGTGGCACCGAGTCG | |
| | | | | | GTGCTTTT | |
| 43030260 | 1 | TTGGTCCTACAGGCC | 579 | AGG | TTGGTCCTACAGGCCAGCACGTTTTAGAGCTAGAAATAGCAAGTTA | 842 |
| | | AGCAC | | | AAATAAGGCTAGTCCGTTATCAACTTGAAAAAGTGGCACCGAGTCG | |
| | | | | | GTGCTTTT | |
| 43030262 | −1 | TCACTTGGCAACACC | 580 | TGG | TCACTTGGCAACACCTGTGCGTTTTAGAGCTAGAAATAGCAAGTTA | 843 |
| | | TGTGC | | | AAATAAGGCTAGTCCGTTATCAACTTGAAAAAGTGGCACCGAGTCG | |
| | | | | | GTGCTTTT | |
| 43030277 | −1 | CTGGGCACATGGGCT | 581 | TGG | CTGGGCACATGGGCTTCACTGTTTTAGAGCTAGAAATAGCAAGTTA | 844 |
| | | TCACT | | | AAATAAGGCTAGTCCGTTATCAACTTGAAAAAGTGGCACCGAGTCG | |
| | | | | | GTGCTTTT | |
| 43030287 | −1 | ATCACTGTGCCTGGG | 582 | GGG | ATCACTGTGCCTGGGCACATGTTTTAGAGCTAGAAATAGCAAGTTA | 845 |
| | | CACAT | | | AAATAAGGCTAGTCCGTTATCAACTTGAAAAAGTGGCACCGAGTCG | |
| | | | | | GTGCTTTT | |
| 43030288 | −1 | GATCACTGTGCCTGG | 583 | TGG | GATCACTGTGCCTGGGCACAGTTTTAGAGCTAGAAATAGCAAGTTA | 846 |
| | | GCACA | | | AAATAAGGCTAGTCCGTTATCAACTTGAAAAAGTGGCACCGAGTCG | |
| | | | | | GTGCTTTT | |
| 43030289 | 1 | CAAGTGAAGCCCATG | 584 | AGG | CAAGTGAAGCCCATGTGCCCGTTTTAGAGCTAGAAATAGCAAGTTA | 847 |
| | | TGCCC | | | AAATAAGGCTAGTCCGTTATCAACTTGAAAAAGTGGCACCGAGTCG | |
| | | | | | GTGCTTTT | |
| 43030295 | −1 | TGCCTGTGATCACTG | 585 | GGG | TGCCTGTGATCACTGTGCCTGTTTTAGAGCTAGAAATAGCAAGTTA | 848 |
| | | TGCCT | | | AAATAAGGCTAGTCCGTTATCAACTTGAAAAAGTGGCACCGAGTCG | |
| | | | | | GTGCTTTT | |
| 43030296 | −1 | ATGCCTGTGATCACT | 586 | TGG | ATGCCTGTGATCACTGTGCCGTTTTAGAGCTAGAAATAGCAAGTTA | 849 |
| | | GTGCC | | | AAATAAGGCTAGTCCGTTATCAACTTGAAAAAGTGGCACCGAGTCG | |
| | | | | | GTGCTTTT | |
| 43030304 | 1 | TGCCCAGGCACAGTG | 587 | AGG | TGCCCAGGCACAGTGATCACGTTTTAGAGCTAGAAATAGCAAGTTA | 850 |
| | | ATCAC | | | AAATAAGGCTAGTCCGTTATCAACTTGAAAAAGTGGCACCGAGTCG | |
| | | | | | GTGCTTTT | |
| 43030312 | 1 | CACAGTGATCACAGG | 588 | TGG | CACAGTGATCACAGGCATTCGTTTTAGAGCTAGAAATAGCAAGTTA | 851 |
| | | CATTC | | | AAATAAGGCTAGTCCGTTATCAACTTGAAAAAGTGGCACCGAGTCG | |
| | | | | | GTGCTTTT | |
| 43030313 | 1 | ACAGTGATCACAGGC | 589 | GGG | ACAGTGATCACAGGCATTCTGTTTTAGAGCTAGAAATAGCAAGTTA | 852 |
| | | ATTCT | | | AAATAAGGCTAGTCCGTTATCAACTTGAAAAAGTGGCACCGAGTCG | |
| | | | | | GTGCTTTT | |
| 43030319 | 1 | ATCACAGGCATTCTGGGTGA | 590 | AGG | ATCACAGGCATTCTGGGTGAGTTTTAGAGCTAGAAATAGCAAGTTA | 853 |
| | | | | | AAATAAGGCTAGTCCGTTATCAACTTGAAAAAGTGGCACCGAGTCG | |
| | | | | | GTGCTTTT | |
| 43030320 | 1 | TCACAGGCATTCTGG | 591 | GGG | TCACAGGCATTCTGGGTGAAGTTTTAGAGCTAGAAATAGCAAGTTA | 854 |
| | | GTGAA | | | AAATAAGGCTAGTCCGTTATCAACTTGAAAAAGTGGCACCGAGTCG | |
| | | | | | GTGCTTTT | |
| 43030323 | 1 | CAGGCATTCTGGGTG | 592 | AGG | CAGGCATTCTGGGTGAAGGGGTTTTAGAGCTAGAAATAGCAAGTTA | 855 |
| | | AAGGG | | | AAATAAGGCTAGTCCGTTATCAACTTGAAAAAGTGGCACCGAGTCG | |
| | | | | | GTGCTTTT | |
| 43030332 | 1 | TGGGTGAAGGGAGGC | 593 | AGG | TGGGTGAAGGGAGGCCTGCAGTTTTAGAGCTAGAAATAGCAAGTTA | 856 |
| | | CTGCA | | | AAATAAGGCTAGTCCGTTATCAACTTGAAAAAGTGGCACCGAGTCG | |
| | | | | | GTGCTTTT | |
| 43030333 | 1 | GGGTGAAGGGAGGCC | 594 | GGG | GGGTGAAGGGAGGCCTGCAAGTTTTAGAGCTAGAAATAGCAAGTTA | 857 |
| | | TGCAA | | | AAATAAGGCTAGTCCGTTATCAACTTGAAAAAGTGGCACCGAGTCG | |
| | | | | | GTGCTTTT | |
| 43030335 | −1 | TGCTGGAAATTGGCC | 595 | AGG | TGCTGGAAATTGGCCCTTGCGTTTTAGAGCTAGAAATAGCAAGTTA | 858 |
| | | CTTGC | | | AAATAAGGCTAGTCCGTTATCAACTTGAAAAAGTGGCACCGAGTCG | |
| | | | | | GTGCTTTT | |

Table 10 below provides exemplary target nucleotide sequences of an HNF4α expression control genomic region and the corresponding zinc finger DNA binding domain amino acid sequences and amino acid structures of disrupting agent fusion proteins comprising a zing finger polypeptide and an effector suitable for use in the present invention.

The forward strand of the HNF4α expression control region targeted by the exemplary target nucleotide sequence in Table 10 comprises the nucleotide sequence of:

(SEQ ID NO: 859)
CTCCGGGAGGGGGTGGGGGTGAGGGAAACAGGAGAATGTGATGGGAAAATC
CGAGATGGAGCCAGCCTGGGCCAGAAACACTGGGAGCTGTGGGAGACGGAG
AGGGGCAGGGTGGGATCACAGGGAGCAGGAGCGGGGAATTGGAGGTGAATC
TGGCCCTCCCAAACTTCCAGTCCATTCTGCTCCCAGGGGAACCGGGAAACT
GCGGGGGAACTGGAAGGGAGCTCCCAGAACAAGGATCCAGAAGATTGGCAT
CTGGGGCCTGGGATTTAGGTTTCTAAATCGTGGGCCATGGGGCAGCCTTAT
CTCTGCAAAAGCATTGAGGGTAGAAGTCAATGATTTGGGAAGTTATTGAAT
TAGGGGATCTCGGAGGTAGGCTGTCAGTGCCTGATAGTATCAGTTAGAATG
CCTGACTTGGGGTGACAATGGCTTGGAGGGGTGGGTGAGTCAAGGGTCAAA
TGAGTGCCCGTGAGTCATGATGCCTGCCTTGTACAATTGATAACTGAACAT
CGGTGAGTTAGGGCCCCAGCAGTTGTAATTAGCACCCCGGGTGTCAGCCAG
AAACCAACAAACAGCCAAATCCCTGCAGCCCCGCCCAGCCTATCCACCGGC
GGGGGACCGATTAACCATTAACCCCCACCCCTCCCCGGCAGAGCCTCCACC
CCTTCACAGAGGCTAGGCCAAGACTCCCAGCAGATCTTCCCAGAGGACGGT
TTGAAAGGAAGGCAGAGAGGGCACTGGGAGGAGGCAGTGGGAGGGCGGAGG
GCGGGGGCCTTCGGGGTGGGCGCCCAGGGTAGGGCAGGTGGCCGCGGCGTG
GAGGCAGGGAGAATGCGACTCTCCAAAACCCTCGTCGACATGGACATGGCC
GACTACAGTGCTGCACTGGACCCAGCCTACACCACCCTGGAATTTGAGAAT
GTGCAGGTGTTGACGATGGGCAATGGTAGGTGGGGGCAGATGTGCCCAGGT
GTGCCAGTGGGGGCAGGTGTGCCTGGGTCCAGGAGCAGATCTTTGGCACTC
AACTTTGGGGTGGGAGGAGAATGATACAAAATGGTAGGTTGGTCCTACAGG
CCAGCACAGGTGTTGCCAAGTGAAGCCCATGTGCCCAGGCACAGTGATCAC
AGGCATTCTGGGTGAAGGGAGGCCTGCAAGGGCCAATTTCCAGCAAAAGTT
GAT

The reverse strand of the HNF4α expression control region targeted by the exemplary target nucleotide sequence in Table 10 comprises the sequence of:

(SEQ ID NO: 860)
ATCGACTTTTGCTGGAAATTGGCCCTTGCAGGCCTCCCTTCACCCAGAATG
CCTGTGATCACTGTGCCTGGGCACATGGGCTTCACTTGGCAACACCTGTGC
TGGCCTGTAGGACCAACCTACCATTTTGTATCATTCTCCTCCCACCCCAAA
GTTGAGTGCCAAAGATCTGCTCCTGGACCCAGGCACACCTGCCCCCACTGG
CACACCTGGGCACATCTGCCCCCACCTACCATTGCCCATCGTCAACACCTG
CACATTCTCAAATTCCAGGGTGGTGTAGGCTGGGTCCAGTGCAGCACTGTA
GTCGGCCATGTCCATGTCGACGAGGGTTTTGGAGAGTCGCATTCTCCCTGC
CTCCACGCCGCGGCCACCTGCCCTACCCTGGGCGCCCACCCCGAAGGCCCC
CGCCCTCCGCCCTCCCACTGCCTCCTCCCAGTGCCCTCTCTGCCTTCCTTT
CAAACCGTCCTCTGGGAAGATCTGCTGGGAGTCTTGGCCTAGCCTCTGTGA
AGGGGTGGAGGCTCTGCCGGGGAGGGGTGGGGGTTAATGGTTAATCGGTCC
CCCGCCGGTGGATAGGCTGGGCGGGGCTGCAGGGATTTGGCTGTTTGTTGG
TTTCTGGCTGACACCCGGGGTGCTAATTACAACTGCTGGGGCCCTAACTCA
CCGATGTTCAGTTATCAATTGTACAAGGCAGGCATCATGACTCACGGGCAC
TCATTTGACCCTTGACTCACCCACCCCTCCAAGCCATTGTCACCCCAAGTC
AGGCATTCTAACTGATACTATCAGGCACTGACAGCCTACCTCCGAGATCCC
CTAATTCAATAACTTCCCAAATCATTGACTTCTACCCTCAATGCTTTTGCA
GAGATAAGGCTGCCCCATGGCCCACGATTTAGAAACCTAAATCCCAGGCCC
CAGATGCCAATCTTCTGGATCCTTGTTCTGGGAGCTCCCTTCCAGTTCCCC
CGCAGTTTCCCGGTTCCCCTGGGAGCAGAATGGACTGGAAGTTTGGGAGGG
CCAGATTCACCTCCAATTCCCCGCTCCTGCTCCCTGTGATCCCACCCTGCC
CCTCTCCGTCTCCCACAGCTCCCAGTGTTTCTGGCCCAGGCTGGCTCCATC
TCGGATTTTCCCATCACATTCTCCTGTTTCCCTCACCCCCACCCCCTCCCG
GAG

In some embodiments, the linker has the amino acid sequence of THPRAPIPKPFQ (SEQ ID NO: 311). In other embodiments, the linker has the amino acid sequence of TPNPHRRTDPSHKPFQ (SEQ ID NO: 312).

TABLE 10

| Column 1 | | | | | | |
|---|---|---|---|---|---|---|
| Nucleot1de | | | | | | |
| Sequence of | | Column 2 | | | | |
| Target Site | | SEQ ID NO. | Column 4 | | | |
| in HNF4α | Column 3 | (corresponds | Amino acid Sequence of Zinc | | Column 6 | |
| Expression | Target | to sequences | Finger DNA Binding Domain | Column 5 | Amino Acid Sequence Structure of Zinc Finger DNA Binding | Column 7 |
| Control | sequence | in Columns 1 | Polypeptides Targeting the | SEQ ID | Domain Polypeptides Targeting the Target Site in Column 1 | SEQ ID |
| Region | with space | and 3) | Target Site in Column 1 | NO: | (see Table 1B above and description thereof) | NO: |
| agatggagc | AGA TGG | 861 | LEPGEKPYKCPECGKSFSTSHSL | 1410 | X₁X₂X₃X₄X₅X₆X₇X₈X₉CX₁₀X₁₁CX₁₂X₁₃X₁₄X₁₅X₁₆TSHSLTEHX₁₇X₁₈X₁₉ | 1959 |
| cagcctggg | AGC CAG | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDKLVRHX₁₇X₁₈ | |
| cca | CCT ggg | | RSDKLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TKNSLTEHX₁₇ | |
| | CCA | | GKSFSTKNSLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RADNLTEH | |
| | | | KCPECGKSFSRADNLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆ERSHLR | |
| | | | GEKPYKCPECGKSFSERSHLREH | | EHX₁₇X₁₈X₁₉HX₂₀X₂₁X₂₂X₂₃X₂₄X₈X₉CX₁₀X₁₁CX₁₂X₁₃X₁₄X₁₅X₁₆RSDH | |
| | | | QRTHTGEKPYKCPECGKSFSRSD | | LTTHX₁₇X₁₈X₁₉HX₂₀X₂₁X₂₂X₂₃X₂₄X₈X₉CX₁₀X₁₁CX₁₂X₁₃X₁₄X₁₅X₁₆QL | |
| | | | HLTTHQRTHTGEKPYKCPECGKS | | AHLRAHX₁₇X₁₈X₁₉HX₂₀X₂₁X₂₂X₂₃X₂₄X₂₅X₂₆X₂₇X₂₈X₂₉X₃₀ | |
| | | | FSQLAHLRAHQRTHTGEKPTGKK | | | |
| | | | TS | | | |
| cagcctggg | CAG CCT | 862 | LEPGEKPYKCPECGKSFSRNDAL | 1411 | X₁X₂X₃X₄X₅X₆X₇X₈X₉CX₁₀X₁₁CX₁₂X₁₃X₁₄X₁₅X₁₆RNDALTEHX₁₇X₁₈X₁₉ | 1960 |
| ccagaaaca | ggg CCA | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆SPADLTRHX₁₇X₁₈ | |
| ctg | GAA ACA | | SPADLTRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QSSNLVRHX₁₇ | |
| | CTG | | GKSFSQSSNLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSHSLTEH | |
| | | | KCPECGKSFSTSHSLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDKLV | |
| | | | GEKPYKCPECGKSFSRSDKLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆TKNS | |
| | | | QRTHTGEKPYKCPECGKSFSTKN | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆RA | |
| | | | SLTEHQRTHTGEKPYKCPECGKS | | DNLTEHX₁₇X₁₈X₁₉HX₂₀X₂₁X₂₂X₂₃X₂₄X₂₅X₂₆X₂₇X₂₈X₂₉X₃₀ | |
| | | | FSRADNLTEHQRTHTGEKPTGKK | | | |
| | | | TS | | | |
| agccagcct | AGC CAG | 863 | LEPGEKPYKCPECGKSFSSPADL | 1412 | X₁X₂X₃X₄X₅X₆X₇X₈X₉CX₁₀X₁₁CX₁₂X₁₃X₁₄X₁₅X₁₆SPADLTRHX₁₇X₁₈X₁₉ | 1961 |
| gggccagaa | CCT ggg | | TRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QSSNLVRHX₁₇X₁₈ | |
| aca | CCA GAA | | QSSNLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSHSLTEHX₁₇ | |
| | ACA | | GKSFSTSHSLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDKLVRH | |
| | | | KCPECGKSFSRSDKLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TKNSLT | |
| | | | GEKPYKCPECGKSFSTKNSLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆RADN | |
| | | | QRTHTGEKPYKCPECGKSFSRAD | | LTEHX₁₇X₁₈X₁₉HX₂₀X₂₁X₂₂X₂₃X₂₄X₈X₉CX₁₀X₁₁CX₁₂XEX₁₄X₁₅X₁₆ER | |
| | | | NLTEHQRTHTGEKPYKCPECGKS | | SHLREHX₁₇X₁₈X₁₉HX₂₀X₂₁X₂₂X₂₃X₂₄X₂₅X₂₆X₂₇X₂₈X₂₉X₃₀ | |
| | | | FSERSHLREHQRTHTGEKPTGKK | | | |
| | | | TS | | | |
| tggagccag | TGG AGC | 864 | LEPGEKPYKCPECGKSFSQSSNL | 1413 | X₁X₂X₃X₄X₅X₆X₇X₈X₉CX₁₀X₁₁CX₁₂X₁₃X₁₄X₁₅X₁₆QSSNLVRHX₁₇X₁₈X₁₉ | 1962 |
| cctgggcca | CAG CCT | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSHSLTEHX₁₇X₁₈ | |
| gaa | ggg CCA | | TSHSLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDKLVRHX₁₇ | |
| | GAA | | GKSFSRSDKLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TKNSLTEH | |
| | | | KCPECGKSFSTKNSLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RADNLT | |
| | | | GEKPYKCPECGKSFSRADNLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆ERSH | |
| | | | QRTHTGEKPYKCPECGKSFSERS | | LREHX₁₇X₁₈X₁₉HX₂₀X₂₁X₂₂X₂₃X₂₄X₈X₉CX10X₁₁CX₁₂X₁₃X₁₄X₁₅X₁₆RS | |
| | | | HLREHQRTHTGEKPYKCPECGKS | | DHLTTHX₁₇X₁₈X₁₉HX₂₀X₂₁X₂₂X₂₃X₂₄X₂₅X₂₆X₂₇X₂₈X₂₉X₃₀ | |
| | | | FSRSDHLTTHQRTHTGEKPTGKK | | | |
| | | | TS | | | |
| ccgagatgg | CCG AGA | 865 | LEPGEKPYKCPECGKSFSRSDKL | 1414 | X₁X₂X₃X₄X₅X₆X₇X₈X₉CX₁₀X₁₁CX₁₂X₁₃X₁₄X₁₅X₁₆RSDKLVRHX₁₇X₁₈X₁₉ | 1963 |
| agccagcct | TGG AGC | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TKNSLTEHX₁₇X₁₈ | |
| ggg | CAG CCT | | TKNSLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RADNLTEHX₁₇ | |
| | GGG | | GKSFSRADNLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆ERSHLREH | |
| | | | KCPECGKSFSERSHLREHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDHLT | |
| | | | GEKPYKCPECGKSFSRSDHLTTH | | THX₁₇X₁₈X₁₉HX₂₀X₂₁X₂₂X₂₃X₂₄X₈X₉CX₁₀X₁₁CX₁₂X₁₃X₁₄X₁₅X₁₆QLAH | |
| | | | QRTHTGEKPYKCPECGKSFSQLA | | LRAHX₁₇X₁₈X₁₉HX₂₀X₂₁X₂₂X₂₃X₂₄X₈X₉CX₁₀X₁₁CX₁₂X₁₃X₁₄X₁₅X₁₆RN | |
| | | | HLRAHQRTHTGEKPYKCPECGKS | | DTLTEHX₁₇X₁₈X₁₉HX₂₀X₂₁X₂₂X₂₃X₂₄X₂₅X₂₆X₂₇X₂₈X₂₉X₃₀ | |
| | | | FSRNDTLTEHQRTHTGEKPTGKK | | | |
| | | | TS | | | |
| aatccgaga | AGA TGG | 866 | LEPGEKPYKCPECGKSFSTKNSL | 1415 | X₁X₂X₃X₄X₅X₆X₇X₈X₉CX₁₀X₁₁CX₁₂X₁₃X₁₄X₁₅X₁₆TKNSLTEHX₁₇X₁₈X₁₉ | 1964 |
| tggagccag | AAT CCG | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RADNLTEHX₁₇X₁₈ | |
| cct | AGC CAG | | RADNLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆ERSHLREHX₁₇ | |
| | CCT | | GKSFSERSHLREHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDHLTTH | |
| | | | KCPECGKSFSRSDHLTTHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QLAHLR | |
| | | | GEKPYKCPECGKSFSQLAHLRAH | | AHX₁₇X₁₈X₁₉HX₂₀X₂₁X₂₂X₂₃X₂₄X₈X₉CX₁₀X₁₁CX₁₂X₁₃X₁₄X₁₅X₁₆RNDT | |
| | | | QRTHTGEKPYKCPECGKSFSRND | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆TT | |
| | | | TLTEHQRTHTGEKPYKCPECGKS | | GNLTVHX₁₇X₁₈X₁₉HX₂₀X₂₁X₂₂X₂₃X₂₄X₂₅X₂₆X₂₇X₂₈X₂₉X₃₀ | |
| | | | FSTTGNLTVHQRTHTGEKPTGKK | | | |
| | | | TS | | | |
| aggagaatg | AGG AGA | 867 | LEPGEKPYKCPECGKSFSTTGNL | 1416 | X₁X₂X₃X₄X₅X₆X₇X₈X₉CX₁₀X₁₁CX₁₂X₁₃X₁₄X₁₅X₁₆TTGNLTVHX₁₇X₁₈X₁₉ | 1965 |
| tgatgggaa | ATG TGA | | TVHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QSSNLVRHX₁₇X₁₈ | |
| aat | TGG GAA | | QSSNLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDHLTTHX₁₇ | |
| | AAT | | GKSFSRSDHLTTHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QAGHLASH | |
| | | | KCPECGKSFSQAGHLASHQRTHT | | X₁₇X₁₈X₁₉HX₂₀X₂₁X₂₂X₂₃X₂₄X₈X₉CXHX₁₁CX₁₂X₁₃X₁₄X₁₅X₁₆RRDELN | |
| | | | GEKPYKCPECGKSFSRRDELNVH | | VHX₁₇X₁₈X₁₉HX₂₀X₂₁X₂₂X₂₃X₂₄X₈X₉CX₁₀X₁₁CX₁₂X₁₃X₁₄X₁₅X₁₆QLAH | |
| | | | QRTHTGEKPYKCPECGKSFSQLA | | LRAHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | HLRAHQRTHTGEKPYKCPECGKS | | DHLTNHX₁₇X₁₈X₁₉HX₂₀X₂₁X₂₂X₂₃X₂₄X₂₅X₂₆X₂₇X₂₈X₂₉X₃₀ | |
| | | | FSRSDHLTNHQRTHTGEKPTGKK | | | |
| | | | TS | | | |
| aacaggaga | AAC AGG | 868 | LEPGEKPYKCPECGKSFSQSSNL | 1417 | X₁X₂X₃X₄X₅X₆X₇X₈X₉CX₁₀X₁₁CX₁₂X₁₃X₁₄X₁₅X₁₆QSSNLVRHX₁₇X₁₈X₁₉ | 1966 |
| atgtgatgg | AGA ATG | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RDSDHLTTHX₁₇X₁₈ | |
| gaa | TGA TGG | | RSDHLTTHQRTHTGEKPYKCPEC | | X₁₉HX₂₀X₂₁X₂₂X₂₃X₂₄X₈X₉CX₁₀X₁₁CX₁₂X₁₃X₁₄HX₁₅X₁₆QAGHLASHX₁₇ | |
| | GAA | | GKSFSQAGHLASHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RRDELNVH | |
| | | | KCPECGKSFSRRDELNVHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QLAHLR | |
| | | | GEKPYKCPECGKSFSQLAHLRAH | | AHX₁₇X₁₈X₁₉HX₂₀X₂₁X₂₂X₂₃X₂₄X₈X₉CX₁₀X₁₁CX₁₂X₁₃X₁₄X₁₅X₁₆RSDH | |
| | | | QRTHTGEKPYKCPECGKSFSRSD | | LTNHX₁₇X₁₈X₁₉HX₂₀X₂₁X₂₂X₂₃X₂₄X₈X₉CX₁₀X₁₁CX₁₂X₁₃X₁₄X₁₅X₁₆DS | |
| | | | HLTNHQRTHTGEKPYKCPECGKS | | GNLRVHX₁₇X₁₈X₁₉HX₂₀X₂₁X₂₂X₂₃X₂₄X₂₅X₂₆X₂₇X₂₈X₂₉X₃₀ | |
| | | | FSDSGNLRVHQRTHTGEKPTGKK | | | |
| | | | TS | | | |
| tgatgggaa | TGA TGG | 869 | LEPGEKPYKCPECGKSFSRSDHL | 1418 | X₁X₂X₃X₄X₅X₆X₇X₈X₉CX₁₀X₁₁CX₁₂X₁₃X₁₄X₁₅X₁₆RSDHLTTHX₁₇X₁₈X₁₉ | 1967 |
| aatccgaga | GAA AAT | | TTHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QLAHLRAHX₁₇X₁₈ | |
| tgg | CCG AGA | | QLAHLRAHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RNDTLTEHX₁₇ | |
| | TGG | | GKSFSRNDTLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TTGNLTVH | |
| | | | KCPECGKSFSTTGNLTVHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QSSNLV | |
| | | | GEKPYKCPECGKSFSQSSNLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDH | |
| | | | QRTHTGEKPYKCPECGKSFSRSD | | LTTHX₁₇X₁₈X₁₉HX₂₀X₂₁X₂₂X₂₃X₂₄X₈X₉CX₁₀X₁₁CX₁₂X₁₃X₁₄X₁₅X₁₆QA | |
| | | | HLTTHQRTHTGEKPYKCPECGKS | | GHLASHX₁₇X₁₈X₁₉HX₂₀X₂₁X₂₂X₂₃X₂₄X₂₅X₂₆X₂₇X₂₈X₂₉X₃₀ | |
| | | | FSQAGHLASHQRTHTGEKPTGKK | | | |
| | | | TS | | | |
| gaaaatccg | GAA AAT | 870 | LEPGEKPYKCPECGKSFSRADNL | 1419 | X₁X₂X₃X₄X₅X₆X₇X₈X₉CX₁₀X₁₁CX₁₂X₁₃X₁₄X₁₅X₁₆RADNLTEHX₁₇X₁₈X₁₉ | 1968 |
| agatggagc | CCG AGA | | TEHQRTHTGEKPYKCPECGKSFS | | HX₂₀X2022X₂₃X₂₄X₈X₉CX₁₀X₁₁CX₁₂X₁₃X₁₄X₁₅X₁₆ERSHLREHX₁₇X₁₈ | |
| cag | TGG AGC | | ERSHLREHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDHLTTHX₁₇ | |
| | CAG | | GKSFSRSDHLTTHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QLAHLRAH | |
| | | | KCPECGKSFSQLAHLRAHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RNDTLT | |
| | | | GEKPYKCPECGKSFSRNDTLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆TTGN | |
| | | | QRTHTGEKPYKCPECGKSFSTTG | | LTVHX₁₇X₁₈X₁₉HX₂₀X₂₁X₂₂X₂₃X₂₄X₈X₉CX₁₀X₁₁CX₁₂X₁₃X₁₄X₁₅X₁₆QS | |
| | | | NLTVHQRTHTGEKPYKCPECGKS | | SNLVRHX₁₇X₁₈X₁₉HX₂₀X₂₁X₂₂X₂₃X₂₄X₂₅X₂₆X₂₇X₂₈X₂₉X₃₀ | |
| | | | FSQSSNLVRHQRTHTGEKPTGKK | | | |
| | | | TS | | | |
| tgggaaaat | TGG GAA | 871 | LEPGEKPYKCPECGKSFSERSHL | 1420 | X₁X₂X₃X₄X₅X₆X₇X₈X₉CX₁₀X₁₁CX₁₂X₁₃X₁₄X₁₅X₁₆ERSHLREHX₁₇X₁₈X₁₉ | 1969 |
| ccgagatgg | AAT CCG | | REHQRTHTGEKPYKCPECGKSFS | | HX₂₀X2022X₂₃X₂₄X₈X₉C10X₁₁CX₁₂X₁₃X₁₄X₁₅X₁₆RSDHLTTHX₁₇X₁₈ | |
| agc | AGA TGG | | RSDHLTTHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QLAHLRAHX₁₇ | |
| | AGC | | GKSFSQLAHLRAHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RNDTLTEH | |
| | | | KCPECGKSFSRNDTLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TTGNLT | |
| | | | GEKPYKCPECGKSFSTTGNLTVH | | VHX₁₇X₁₈X₁₉HX₂₀X₂₁X₂₂X₂₃X₂₄X₈X₉CX₁₀X₁₁CX₁₂X₁₃X₁₄X₁₅X₁₆QSSN | |
| | | | QRTHTGEKPYKCPECGKSFSQSS | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | NLVRHQRTHTGEKPYKCPECGKS | | DHLTTHX₁₇X₁₈X₁₉HX₂₀X₂₁X₂₂X₂₃X₂₄X₂₅X₂₆X₂₇X₂₈X₂₉X₃₀ | |
| | | | FSRSDHLTTHQRTHTGEKPTGKK | | | |
| | | | TS | | | |
| cctgggcca | CCT ggg | 872 | LEPGEKPYKCPECGKSFSQRAHL | 1421 | X₁X₂X₃X₄X₅X₆X₇X₈X₉CX₁₀X₁₁CX₁₂X₁₃X₁₄X₁₅X₁₆QRAHLERHX₁₇X₁₈X₁₉ | 1970 |
| gaaacactg | CCA GAA | | ERHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RNDALTEHX₁₇X₁₈ | |
| gga | ACA CTG | | RNDALTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆SPADLTRHX₁₇ | |
| | GGA | | GKSFSSPADLTRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QSSNLVRH | |
| | | | KCPECGKSFSQSSNLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSHSLT | |
| | | | GEKPYKCPECGKSFSTSHSLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆RSDK | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆TK | |
| | | | KLVRHQRTHTGEKPYKCPECGKS | | NSLTEHX₁₇X₁₈X₁₉HX₂₀X₂₁X₂₂X₂₃X₂₄X₂₅X₂₆X₂₇X₂₈X₂₉X₃₀ | |
| | | | FSTKNSLTEHQRTHTGEKPTGKK | | | |
| | | | TS | | | |
| ccagaaaca | CCA GAA | 873 | LEPGEKPYKCPECGKSFSRSDEL | 1422 | X₁X₂X₃X₄X₅X₆X₇X₈X₉CX₁₁X₁₁CX₁₂X₁₃X₁₄X₁₅X₁₆RSDELVRHX₁₇X₁₈X₁₉ | 1971 |
| ctgggagct | ACA CTG | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSGELVRHX₁₇X₁₈ | |
| gtg | GGA GCT | | TSGELVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QRAHLERHX₁₇ | |
| | GTG | | GKSFSQRAHLERHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RNDALTEH | |
| | | | KCPECGKSFSRNDALTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆SPADLT | |
| | | | GEKPYKCPECGKSFSSPADLTRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆QSSN | |
| | | | QRTHTGEKPYKCPECGKSFSQSS | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | NLVRHQRTHTGEKPYKCPECGKS | | HSLTEHX₁₇X₁₈X₁₉HX₂₀X₂₁X₂₂X₂₃X₂₄X₂₅X₂₆X₂₇X₂₈X₂₉X₃₀ | |
| | | | FSTSHSLTEHQRTHTGEKPTGKK | | | |
| | | | TS | | | |
| gggccagaa | ggg CCA | 874 | LEPGEKPYKCPECGKSFSTSGEL | 1423 | X₁X₂X₃X₄X₅X₆X₇X₈X₉CX₁₀X₁₁CX₁₂X₁₃X₁₄X₁₅X₁₆TSGELVRHX₁₇X₁₈X₁₉ | 1972 |
| acactggga | GAA ACA | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QRAHLERHX₁₇X₁₈ | |
| gct | CTG GGA | | QRAHLERHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RNDALTEHX₁₇ | |
| | GCT | | GKSFSRNDALTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆SPADLTRH | |
| | | | KCPECGKSFSSPADLTRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QSSNLV | |
| | | | GEKPYKCPECGKSFSQSSNLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆TSHS | |
| | | | QRTHTGEKPYKCPECGKSFSTSH | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | SLTEHQRTHTGEKPYKCPECGKS | | DKLVRHX₁₇X₁₈X₁₉HX₂₀X₂₁X₂₂X₂₃X₂₄X₂₅X₂₆X₂₇X₂₈X₂₉X₃₀ | |
| | | | FSRSDKLVRHQRTHTGEKPTGKK | | | |
| | | | TS | | | |
| gagggaaac | GAG GGA | 875 | LEPGEKPYKCPECGKSFSQAGHL | 1424 | X₁X₂X₃X₄X₅X₆X₇X₈X₉CX₁₀X₁₁CX₁₂X₁₃X₁₄X₁₅X₁₆QAGHLASHX₁₇X₁₈X₁₉ | 1973 |
| aggagaatg | AAC AGG | | ASHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RRDELNVHX₁₇X₁₈ | |
| tga | AGA ATG | | RRDELNVHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QLAHLRAHX₁₇ | |
| | TGA | | GKSFSQLAHLRAHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDHLTNH | |
| | | | KCPECGKSFSRSDHLTNHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DSGNLR | |
| | | | GEKPYKCPECGKSFSDSGNLRVH | | VHX₁₇X₁₈X₁₉HX₂₀X₂₁X₂₂X₂₃X₂₄X₈X₉CX₁₀X₁₁CX₁₂X₁₃X₁₄X₁₅X₁₆QRAH | |
| | | | QRTHTGEKPYKCPECGKSFSQRA | | LERHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | HLERHQRTHTGEKPYKCPECGKS | | DNLVRHX₁₇X₁₈X₁₉HX₂₀X₂₁X₂₂X₂₃X₂₄X₂₅X₂₆X₂₇X₂₈X₂₉X₃₀ | |
| | | | FSRSDNLVRHQRTHTGEKPTGKK | | | |
| | | | TS | | | |
| ggaaacagg | GGA AAC | 876 | LEPGEKPYKCPECGKSFSRSDHL | 1425 | X₁X₂X₃X₄X₅X₆X₇X₈X₉CX₁₀X₁₁CX₁₂X₁₃X₁₄X₁₅X₁₆RSDHLTTHX₁₇X₁₈X₁₉ | 1974 |
| agaatgtga | AGG AGA | | TTHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QAGHLASHX₁₇X₁₈ | |
| tgg | ATG TGA | | QAGHLASHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RRDELNVHX₁₇ | |
| | TGG | | GKSFSRRDELNVHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QLAHLRAH | |
| | | | KCPECGKSFSQLAHLRAHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDHLT | |
| | | | GEKPYKCPECGKSFSRSDHLTNH | | NHX₁₇X₁₈X₁₉HX₂₀X₂₁X₂₂X₂₃X₂₄X₈X₉CX₁₀X₁₁CX₁₂X₁₃X₁₄X₁₅X₁₆DSGN | |
| | | | QRTHTGEKPYKCPECGKSFSDSG | | LRVHX₁₇X₁₈X₁₉HX₂₀X₂₁X₂₂X₂₃X₂₄X₈X₉CX₁₀X₁₁CX₁₂X₁₃X₁₄X₁₅X₁₆QR | |
| | | | NLRVHQRTHTGEKPYKCPECGKS | | AHLERHX₁₇X₁₈X₁₉HX₂₀X₂₁X₂₂X₂₃X₂₄X₂₅X₂₆X₂₇X₂₈X₂₉X₃₀ | |
| | | | FSQRAHLERHQRTHTGEKPTGKK | | | |
| | | | TS | | | |
| atgtgatgg | ATG TGA | 877 | LEPGEKPYKCPECGKSFSQLAHL | 1426 | X₁X₂X₃X₄X₅X₆X₇X₈X₉CX₁₀X₁₁CX₁₂X₁₃X₁₄X₁₅X₁₆QLAHLRAHX₁₇X₁₈X₁₉ | 1975 |
| gaaaatccg | TGG GAA | | RAHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RNDTLTEHX₁₇X₁₈ | |
| aga | AAT CCG | | RNDTLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TTGNLTVHX₁₇ | |
| | AGA | | GKSFSTTGNLTVHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QSSNLVRH | |
| | | | KCPECGKSFSQSSNLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDHLY | |
| | | | GEKPYKCPECGKSFSRSDHLTTH | | THX₁₇X₁₈X₁₉HX₂₀X₂₁X₂₂X₂₃X₂₄X₈X₉CX₁₀X₁₁CX₁₂X₁₃X₁₄X₁₅X₁₆QAGH | |
| | | | QRTHTGEKPYKCPECGKSFSQAG | | LASHX₁₇X₁₈X₁₉HX₂₀X₂₁X₂₂X₂₃X₂₄X₈X₉CX₁₀X₁₁CX₁₂X₁₃X₁₄X₁₅X₁₆RR | |
| | | | HLASHQRTHTGEKPYKCPECGKS | | DELNVHX₁₇X₁₈X₁₉HX₂₀X₂₁X₂₂X₂₃X₂₄X₂₅X₂₆X₂₇X₂₈X₂₉X₃₀ | |
| | | | FSRRDELNVHQRTHTGEKPTGKK | | | |
| | | | TS | | | |
| agaatgtga | AGA ATG | 878 | LEPGEKPYKCPECGKSFSRNDTL | 1427 | X₁X₂X₃X₄X₅X₆X₇X₈X₉CX₁₀X₁₁CX₁₂X₁₃X₁₄X₁₅X₁₆RNDTLTEHX₁₇X₁₈X₁₉ | 1976 |
| tgggaaaat | TGA TGG | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TTGNLTVHX₁₇X₁₈ | |
| ccg | GAA AT | | TTGNLTVHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QSSNLVRHX₁₇ | |
| | CCG | | GKSFSQSSNLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDHLTTH | |
| | | | KCPECGKSFSRSDHLTTHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QAGHLA | |
| | | | GEKPYKCPECGKSFSQAGHLASH | | SHX₁₇X₁₈X₁₉HX₂₀X₂₁X₂₂X₂₃X₂₄X₈X₉CX₁₀X₁₁CX₁₂X₁₃X₁₄X₁₅X₁₆RRDE | |
| | | | QRTHTGEKPYKCPECGKSFSRRD | | LNVHX₁₇X₁₈X₁₉HX₂₀X₂₁X₂₂X₂₃X₂₄X₈X₉CX₁₀X₁₁CX₁₂X₁₃X₁₄X₁₅X₁₆QL | |
| | | | ELNVHQRTHTGEKPYKCPECGKS | | AHLRAHX₁₇X₁₈X₁₉HX₂₀X₂₁X₂₂X₂₃X₂₄X₂₅X₂₆X₂₇X₂₈X₂₉X₃₀ | |
| | | | FSQLAHLRAHQRTHTGEKPTGKK | | | |
| | | | TS | | | |
| gaaacactg | GAA ACA | 879 | LEPGEKPYKCPECGKSFSQRAHL | 1428 | X₁X₂X₃X₄X₅X₆X₇X₈X₉CX₁₀X₁₁CX₁₂X₁₃X₁₄X₁₅X₁₆QRAHLERHX₁₇X₁₈X₁₉ | 1977 |
| ggagctgtg | CTG GGA | | ERHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDELVRHX₁₇X₁₈ | |
| gga | GCT GTG | | RSDELVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSGELVRHX₁₇ | |
| | GGA | | GKSFSTSGELVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QRAHLERH | |
| | | | KCPECGKSFSQRAHLERHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RNDALT | |
| | | | GEKPYKCPECGKSFSRNDALTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆SPAD | |
| | | | QRTHTGEKPYKCPECGKSFSSPA | | LTRHX₁₇X₁₈X₁₉HX₂₀X₂₁X₂₂X₂₃X₂₄X₈X₉CX₁₀X₁₁CX₁₂X₁₃X₁₄X₁₅X₁₆QS | |
| | | | DLTRHQRTHTGEKPYKCPECGKS | | SNLVRHX₁₇X₁₈X₁₉HX₂₀X₂₁X₂₂X₂₃X₂₄X₂₅X₂₆X₂₇X₂₈X₂₉X₃₀ | |
| | | | FSQSSNLVRHQRTHTGEKPTGKK | | | |
| | | | TS | | | |
| gtgggagac | GTG GGA | 880 | LEPGEKPYKCPECGKSFSRADNL | 1429 | X₁X₂X₃X₄X₅X₆X₇X₈X₉CX₁₀X₁₁CX₁₂X₁₃X₁₄X₁₅X₁₆RADNLTEHX₁₇X₁₈X₁₉ | 1978 |
| ggagagggg | GAC GGA | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDKLVRHX₁₇X₁₈ | |
| cag | GAG ggg | | RSDKLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDNLVRHX₁₇ | |
| | CAG | | GKSFSRSDNLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QRAHLERH | |
| | | | KCPECGKSFSQRAHLERHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DPGNLV | |
| | | | GEKPYKCPECGKSFSDPGNLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆QRAH | |
| | | | QRTHTGEKPYKCPECGKSFSQRA | | LERHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | HLERHQRTHTGEKPYKCPECGKS | | DELVRHX₁₇X₁₈X₁₉HX₂₀X₂₁X₂₂X₂₃X₂₄X₂₅X₂₆X₂₇X₂₈X₂₉X₃₀ | |
| | | | FSRSDELVRHQRTHTGEKPTGKK | | | |
| | | | TS | | | |
| acactggga | ACA CTG | 881 | LEPGEKPYKCPECGKSFSDPGNL | 1430 | X₁X₂X₃X₄X₅X₆X₇X₈X₉CX₁₀X₁₁CX₁₂X₁₃X₁₄X₁₅X₁₆DPGNLVRHX₁₇X₁₈X₁₉ | 1979 |
| gctgtggga | GGA GCT | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QRAHLERHX₁₇X₁₈ | |
| gac | GTG GGA | | QRAHLERHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDELVRHX₁₇ | |
| | GAC | | GKSFSRSDELVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSGELVRH | |
| | | | KCPECGKSFSTSGELVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QRAHLE | |
| | | | GEKPYKCPECGKSFSQRAHLERH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RNDA | |
| | | | QRTHTGEKPYKCPECGKSFSRND | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆SP | |
| | | | ALTEHQRTHTGEKPYKCPECGKS | | ADLTRHX₁₇X₁₈X₁₉HX₂₀X₂₁X₂₂X₂₃X₂₄X₂₅X₂₆X₂₇X₂₈X₂₉X₃₀ | |
| | | | FSSPADLTRHQRTHTGEKPTGKK | | | |
| | | | TS | | | |
| gctgtggga | GCT GTG | 882 | LEPGEKPYKCPECGKSFSRSDKL | 1431 | X₁X₂X₃X₄X₅X₆X₇X₈X₉CX₁₀X₁₁CX₁₂X₁₃X₁₄X₁₅X₁₆RSDKLVRHX₁₇X₁₈X₁₉ | 1980 |
| gacggagag | GGA GAC | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDNLVRHX₁₇X₁₈ | |
| ggg | GGA GAG | | RSDNLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QRAHLERHX₁₇ | |
| | GGG | | GKSFSQRAHLERHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DPGNLVRH | |
| | | | KCPECGKSFSDPGNLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QRAHLE | |
| | | | GEKPYKCPECGKSFSQRAHLERH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDE | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | ELVRHQRTHTGEKPYKCPECGKS | | GELVRHX₁₇X₁₈X₁₉HX₂₀X₂₁X₂₂X₂₃X₂₄X₂₅X₂₆X₂₇X₂₈X₂₉X₃₀ | |
| | | | FSTSGELVRHQRTHTGEKPTGKK | | | |
| | | | TS | | | |
| ctgggagct | CTG GGA | 883 | LEPGEKPYKCPECGKSFSQRAHL | 1432 | X₁X₂X₃X₄X₅X₆X₇X₈X₉CX₁₀X₁₁CX₁₂X₁₃X₁₄X₁₅X₁₆QRAHLERHX₁₇X₁₈X₁₉ | 1981 |
| gtgggagac | GCT GTG | | ERHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆DPGNLVRHX₁₇X₁₈ | |
| gga | GGA GAC | | DPGNLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QRAHLERHX₁₇ | |
| | GGA | | GKSFSQRAHLERHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDELVRH | |
| | | | KCPECGKSFSRSDELVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSGELV | |
| | | | GEKPYKCPECGKSFSTSGELVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆QRAH | |
| | | | QRTHTGEKPYKCPECGKSFSQRA | | LERHX₁₇X₁₈X₁₉HX₂₀X₂₁X₂₂X₂₃X₂₄X₈X₉CX₁₀X₁₁CX₁₂X₁₃X₁₄X₁₅X₁₆RN | |
| | | | HLERHQRTHTGEKPYKCPECGKS | | DALTEHX₁₇X₁₃X₁₉HX₂₀X₂₁X₂₂X₂₃X₂₄X₂₅X₂₆X₂₇X₂₈X₂₉X₃₀ | |
| | | | FSRNDALTEHQRTHTGEKPTGKK | | | |
| | | | TS | | | |
| ggagctgtg | GGA GCT | 884 | LEPGEKPYKCPECGKSFSRSDNL | 1433 | X₁X₂X₃X₄X₅X₆X₇X₈X₉CX₁₀X₁₁CX₁₂X₁₃X₁₄X₁₅X₁₆RSDNLVRHX₁₇X₁₈X₁₉ | 1982 |
| ggagacgga | GTG GGA | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QRAHLERHX₁₇X₁₈ | |
| gag | GAC GGA | | QRAHLERHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DPGNLVRHX₁₇ | |
| | GAG | | GKSFSDPGNLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QRAHLERH | |
| | | | KCPECGKSFSQRAHLERHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDELV | |
| | | | GEKPYKCPECGKSFSRSDELVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆TSGE | |
| | | | QRTHTGEKPYKCPECGKSFSTSG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆QR | |
| | | | ELVRHQRTHTGEKPYKCPECGKS | | AHLERHX₁₇X₁₃X₁₉HX₂₀X₂₁X₂₂X₂₃X₂₄X₂₅X₂₆X₂₇X₂₈X₂₉X₃₀ | |
| | | | FSQRAHLERHQRTHTGEKPTGKK | | | |
| | | | TS | | | |
| gggggtgag | GGG GGT | 885 | LEPGEKPYKCPECGKSFSQLAHL | 1434 | X₁X₂X₃X₄X₅X₆X₇X₈X₉CX₁₀X₁₁CX₁₂X₁₃X₁₄X₁₅X₁₆QLAHLRAHX₁₇X₁₈X₁₉ | 1983 |
| ggaaacagg | GAG GGA | | RAHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDHLTNHX₁₇X₁₈ | |
| aga | AAC AGG | | RSDHLTNHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DSGNLRVHX₁₇ | |
| | AGA | | GKSFSDSGNLRVHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QRAHLERH | |
| | | | KCPECGKSFSQRAHLERHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDNLV | |
| | | | GEKPYKCPECGKSFSRSDNLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆TSGH | |
| | | | QRTHTGEKPYKCPECGKSFSTSG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | HLVRHQRTHTGEKPYKCPECGKS | | DKLVRHX₁₇X₁₈X₁₉HX₂₀X₂₁X₂₂X₂₃X₂₄X₂₅X₂₆X₂₇X₂₈X₂₉X₃₀ | |
| | | | FSRSDKLVRHQRTHTGEKPTGKK | | | |
| | | | TS | | | |
| gacggagag | GAC GGA | 886 | LEPGEKPYKCPECGKSFSRSDKL | 1435 | X₁X₂X₃X₄X₅X₆X₇X₈X₉CX₁₀X₁₁CX₁₂X₁₃X₁₄X₁₅X₁₆RSDKLVRHX₁₇X₁₈X₁₉ | 1984 |
| gggcagggt | GAG ggg | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSGHLVRHX₁₇X₁₈ | |
| ggg | CAG GGT | | TSGHLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RADNLTEHX₁₇ | |
| | GGG | | GKSFSRADNLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDKLVRH | |
| | | | KCPECGKSFSRSDKLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDNLV | |
| | | | GEKPYKCPECGKSFSRSDNLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆QRAH | |
| | | | QRTHTGEKPYKCPECGKSFSQRA | | LERHX₁₇X₁₈X₁₉HX₂₀X₂₁X₂₂X₂₃X₂₄X₈X₉CX₁₀X₁₁CX₁₂X₁₃X₁₄X₁₅X₁₆DP | |
| | | | HLERHQRTHTGEKPYKCPECGKS | | GNLVRHX₁₇X₁₈X₁₉HX₂₀X₂₁X₂₂X₂₃X₂₄X₂₅X₂₆X₂₇X₂₈X₂₉X₃₀ | |
| | | | FSDPGNLVRHQRTHTGEKPTGKK | | | |
| | | | TS | | | |
| ggtgaggga | GGT GAG | 887 | LEPGEKPYKCPECGKSFSRRDEL | 1436 | X₁X₂X₃X₄X₅X₆X₇X₈X₉CX₁₀X₁₁CX₁₂X₁₃X₁₄X₁₅X₁₆RRDELNVHX₁₇X₁₈X₁₉ | 1985 |
| aacaggaga | GGA AAC | | NVHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QLAHLRAHX₁₇X₁₈ | |
| atg | AGG AGA | | QLAHLRAHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDHLTNHX₁₇ | |
| | ATG | | GKSFSRSDHLTNHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DSGNLRVH | |
| | | | KCPECGKSFSDSGNLRVHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QRAHLE | |
| | | | GEKPYKCPECGKSFSQRAHLERH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDN | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | NLVRHQRTHTGEKPYKCPECGKS | | GHLVRHX₁₇X₁₈X₁₉HX₂₀X₂₁X₂₂X₂₃X₂₄X₂₅X₂₆X₂₇X₂₈X₂₉X₃₀ | |
| | | | FSTSGHLVRHQRTHTGEKPTGKK | | | |
| | | | TS | | | |
| ggagacgga | GGA GAC | 888 | LEPGEKPYKCPECGKSFSTSGHL | 1437 | X₁X₂X₃X₄X₅X₆X₇X₈X₉CX₁₀X₁₁CX₁₂X₁₃X₁₄X₁₅X₁₆TSGHLVRHX₁₇X₁₈X₁₉ | 1986 |
| gaggggcag | GGA GAG | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RADNLTEHX₁₇X₁₈ | |
| ggt | ggg CAG | | RADNLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDKLVRHX₁₇ | |
| | GGT | | GKSFSRSDKLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDNLVRH | |
| | | | KCPECGKSFSRSDNLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QRAHLE | |
| | | | GEKPYKCPECGKSFSQRAHLERH | | RHX₁₇X₁₈X₁₉HX₂₀X₂₁X₂₂X₂₃X₂₄X₈X₉CX₁₀X₁₁CX₁₂X₁₃X₁₄X₁₅X₁₆DPGN | |
| | | | QRTHTGEKPYKCPECGKSFSDPG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆QR | |
| | | | NLVRHQRTHTGEKPYKCPECGKS | | AHLERHX₁₇X₁₈X₁₉HX₂₀X₂₁X₂₂X₂₃X₂₄X₂₅X₂₆X₂₇X₂₈X₂₉X₃₀ | |
| | | | FSQRAHLERHQRTHTGEKPTGKK | | | |
| | | | TS | | | |
| gggggtggg | GGG GGT | 889 | LEPGEKPYKCPECGKSFSDSGNL | 1438 | X₁X₂X₃X₄X₅X₆X₇X₈X₉CX₁₀X₁₁CX₁₂X₁₃X₁₄X₁₅X₁₆DSGNLRVHX₁₇X₁₈X₁₉ | 1987 |
| ggtgaggga | GGG GGT | | RVHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QRAHLERHX₁₇X₁₈ | |
| aac | GAG GGA | | QRAHLERHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDNLVRHX₁₇ | |
| | AAC | | GKSFSRSDNLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSGHLVRH | |
| | | | KCPECGKSFSTSGHLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDKLV | |
| | | | GEKPYKCPECGKSFSRSDKLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆TSGH | |
| | | | QRTHTGEKPYKCPECGKSFSTSG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | HLVRHQRTHTGEKPYKCPECGKS | | DKLVRHX₁₇X₁₈X₁₉HX₂₀X₂₁X₂₂X₂₃X₂₄X₂₅X₂₆X₂₇X₂₈X₂₉X₃₀ | |
| | | | FSRSDKLVRHQRTHTGEKPTGKK | | | |
| | | | TS | | | |
| ggtgggggt | GGT GGG | 890 | LEPGEKPYKCPECGKSFSRSDHL | 1439 | X₁X₂X₃X₄X₅X₆X₇X₈X₉CX₁₀X₁₁CX₁₂X₁₃X₁₄X₁₅X₁₆RSDHLTNHX₁₇X₁₈X₁₉ | 1988 |
| gagggaaac | GGT GAG | | TNHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆DSGNLRVHX₁₇X₁₈ | |
| agg | GGA AAC | | DSGNLRVHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QRAHLERHX₁₇ | |
| | AGG | | GKSFSQRAHLERHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDNLVRH | |
| | | | KCPECGKSFSRSDNLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSGHLV | |
| | | | GEKPYKCPECGKSFSTSGHLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDK | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | KLVRHQRTHTGEKPYKCPECGKS | | GHLVRHX₁₇X₁₈X₁₉HX₂₀X₂₁X₂₂X₂₃X₂₄X₂₅X₂₆X₂₇X₂₈X₂₉X₃₀ | |
| | | | FSTSGHLVRHQRTHTGEKPTGKK | | | |
| | | | TS | | | |
| ggagggggt | GGA GGG | 891 | LEPGEKPYKCPECGKSFSQRAHL | 1440 | X₁X₂X₃X₄X₅X₆X₇X₈X₉CX₁₀X₁₁CX₁₂X₁₃X₁₄X₁₅X₁₆QRAHLERHX₁₇X₁₈X₁₉ | 1989 |
| gggggtgag | GGT GGG | | ERHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDNLVRHX₁₇X₁₈ | |
| gga | GGT GAG | | RSDNLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSGHLVRHX₁₇ | |
| | GGA | | GKSFSTSGHLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDKLVRH | |
| | | | KCPECGKSFSRSDKLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSGHLV | |
| | | | GEKPYKCPECGKSFSTSGHLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDK | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆QR | |
| | | | KLVRHQRTHTGEKPYKCPECGKS | | AHLERHX₁₇X₁₈X₁₉HX₂₀X₂₁X₂₂X₂₃X₂₄X₂₅X₂₆X₂₇X₂₈X₂₉X₃₀ | |
| | | | FSQRAHLERHQRTHTGEKPTGKK | | | |
| | | | TS | | | |
| ccgggaggg | CCG GGA | 892 | LEPGEKPYKCPECGKSFSRSDNL | 1441 | X₁X₂X₃X₄X₅X₆X₇X₈X₉CX₁₀X₁₁CX₁₂X₁₃X₁₄X₁₅X₁₆RSDNLVRHX₁₇X₁₈X₁₉ | 1990 |
| ggtgggggt | GGG GGT | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSGHLVRHX₁₇X₁₈ | |
| gag | GGG GGT | | TSGHLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDKLVRHX₁₇ | |
| | GAG | | GKSFSRSDKLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSGHLVRH | |
| | | | KCPECGKSFSTSGHLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDKLV | |
| | | | GEKPYKCPECGKSFSRSDKLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆QRAH | |
| | | | QRTHTGEKPYKCPECGKSFSQRA | | LERHX₁₇X₁₈X₁₉HX₂₀X₂₁X₂₂X₂₃X₂₄X₈X₉CX₁₀X₁₁CX₁₂X₁₃X₁₄X₁₅X₁₆RN | |
| | | | HLERHQRTHTGEKPYKCPECGKS | | DTLTEHX₁₇X₁₈X₁₉HX₂₀X₂₁X₂₂X₂₃X₂₄X₂₅X₂₆X₂₇X₂₈X₂₉X₃₀ | |
| | | | FSRNDTLTEHQRTHTGEKPTGKK | | | |
| | | | TS | | | |
| cgggagggg | CGG GAG | 893 | LEPGEKPYKCPECGKSFSRSDHL | 1442 | X₁X₂X₃X₄X₅X₆X₇X₈X₉CX₁₀X₁₁CX₁₂X₁₃X₁₄X₁₅X₁₆RSDHLTNHX₁₇X₁₈X₁₉ | 1991 |
| gtgggggtg | GGG GTG | | TNHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDELVRHX₁₇X₁₈ | |
| agg | GGG gtg | | RSDELVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDKLVRHX₁₇ | |
| | AGG | | GKSFSRSDKLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDELVRH | |
| | | | KCPECGKSFSRSDELVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDKLV | |
| | | | GEKPYKCPECGKSFSRSDKLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDN | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | NLVRHQRTHTGEKPYKCPECGKS | | DKLTEHX₁₇X₁₈X₁₉HX₂₀X₂₁X₂₂X₂₃X₂₄X₂₅X₂₆X₂₇X₂₈X₂₉X₃₀ | |
| | | | FSRSDKLTEHQRTHTGEKPTGKK | | | |
| | | | TS | | | |
| gagggggtg | GAG GGG | 894 | LEPGEKPYKCPECGKSFSQSSNL | 1443 | X₁X₂X₃X₄X₅X₆X₇X₈X₉CX₁₀X₁₁CX₁₂X₁₃X₁₄X₁₅X₁₆QSSNLVRHX₁₇X₁₈X₁₉ | 1992 |
| ggggtgagg | GTG GGG | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDHLTNHX₁₇X₁₈ | |
| gaa | gtg AGG | | RSDHLTNHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDELVRHX₁₇ | |
| | GAA | | GKSFSRSDELVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDKLVRH | |
| | | | KCPECGKSFSRSDKLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDELV | |
| | | | GEKPYKCPECGKSFSRSDELVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDK | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | KLVRHQRTHTGEKPYKCPECGKS | | DNLVRHX₁₇X₁₈X₁₉HX₂₀X₂₁X₂₂X₂₃X₂₄X₂₅X₂₆X₂₇X₂₈X₂₉X₃₀ | |
| | | | FSRSDNLVRHQRTHTGEKPTGKK | | | |
| | | | TS | | | |
| ggggtgggg | GGG GTG | 895 | LEPGEKPYKCPECGKSFSSPADL | 1444 | X₁X₂X₃X₄X₅X₆X₇X₈X₉CX₁₀X₁₁CX₁₂X₁₃X₁₄X₁₅X₁₆SPADLTRHX₁₇X₁₈X₁₉ | 1993 |
| gtgagggaa | GGG gtg | | TRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QSSNLVRHX₁₇X₁₈ | |
| aca | AGG GAA | | QSSNLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDHLTNHX₁₇ | |
| | ACA | | GKSFSRSDHLTNHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDELVRH | |
| | | | KCPECGKSFSRSDELVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDKLV | |
| | | | GEKPYKCPECGKSFSRSDKLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDE | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | ELVRHQRTHTGEKPYKCPECGKS | | DKLVRHX₁₇X₁₈X₁₉HX₂₀X₂₁X₂₂X₂₃X₂₄X₂₅X₂₆X₂₇X₂₈X₂₉X₃₀ | |
| | | | FSRSDKLVRHQRTHTGEKPTGKK | | | |
| | | | TS | | | |
| gtgggggtg | GTG GGG | 896 | LEPGEKPYKCPECGKSFSQRAHL | 1445 | X₁X₂X₃X₄X₅X₆X₇X₈X₉CX₁₀X₁₁CX₁₂X₁₃X₁₄X₁₅X₁₆QRAHLERHX₁₇X₁₈X₁₉ | 1994 |
| agggaaaca | gtg AGG | | ERHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆SPADLTRHX₁₇X₁₈ | |
| gga | GAA ACA | | SPADLTRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QSSNLVRHX₁₇ | |
| | GGA | | GKSFSQSSNLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDHLTNH | |
| | | | KCPECGKSFSRSDHLTNHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDELV | |
| | | | GEKPYKCPECGKSFSRSDELVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDK | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | KLVRHQRTHTGEKPYKCPECGKS | | DELVRHX₁₇X₁₈X₁₉HX₂₀X₂₁X₂₂X₂₃X₂₄X₂₅X₂₆X₂₇X₂₈X₂₉X₃₀ | |
| | | | FSRSDELVRHQRTHTGEKPTGKK | | | |
| | | | TS | | | |
| ggggtgagg | GGG gtg | 897 | LEPGEKPYKCPECGKSFSQSSNL | 1446 | X₁X₂X₃X₄X₅X₆X₇X₈X₉CX₁₀X₁₁CX₁₂X₁₃X₁₄X₁₅X₁₆QSSNLVRHX₁₇X₁₈X₁₉ | 1995 |
| gaaacagga | AGG GAA | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QRAHLERHX₁₇X₁₈ | |
| gaa | ACA GGA | | QRAHLERHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆SPADLTRHX₁₇ | |
| | GAA | | GKSFSSPADLTRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QSSNLVRH | |
| | | | KCPECGKSFSQSSNLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDHLT | |
| | | | GEKPYKCPECGKSFSRSDHLTNH | | NHX₁₇X₁₈X₁₉HX₂₀X₂₁X₂₂X₂₃X₂₄X₈X₉CX₁₀X₁₁CX₁₂X₁₃X₁₄X₁₅X₁₆RSDE | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | ELVRHQRTHTGEKPYKCPECGKS | | DKLVRHX₁₇X₁₈X₁₉HX₂₀X₂₁X₂₂X₂₃X₂₄X₂₅X₂₆X₂₇X₂₈X₂₉X₃₀ | |
| | | | FSRSDKLVRHQRTHTGEKPTGKK | | | |
| | | | TS | | | |
| gggtgaggg | GGG TGA | 898 | LEPGEKPYKCPECGKSFSTTGNL | 1447 | X₁X₂X₃X₄X₅X₆X₇X₈X₉CX₁₀X₁₁CX₁₂X₁₃X₁₄X₁₅X₁₆TTGNLTVHX₁₇X₁₈X₁₉ | 1996 |
| aaacaggag | ggg AAA | | TVHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDNLVRHX₁₇X₁₈ | |
| aat | CAG gag | | RSDNLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RADNLTEHX₁₇ | |
| | AAT | | GKSFSRADNLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QRANLRAH | |
| | | | KCPECGKSFSQRANLRAHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDKLV | |
| | | | GEKPYKCPECGKSFSRSDKLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆QAGH | |
| | | | QRTHTGEKPYKCPECGKSFSQAG | | LASHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | HLASHQRTHTGEKPYKCPECGKS | | DKLVRHX₁₇X₁₈X₁₉HX₂₀X₂₁X₂₂X₂₃X₂₄X₂₅X₂₆X₂₇X₂₈X₂₉X₃₀ | |
| | | | FSRSDKLVRHQRTHTGEKPTGKK | | | |
| | | | TS | | | |
| gggtggggg | GGG TGG | 899 | LEPGEKPYKCPECGKSFSRADNL | 1448 | X₁X₂X₃X₄X₅X₆X₇X₈X₉CX₁₀X₁₁CX₁₂X₁₃X₁₄X₁₅X₁₆RADNLTEHX₁₇X₁₈X₁₉ | 1997 |
| tgagggaaa | GGG TGA | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QRANLRAHX₁₇X₁₈ | |
| cag | ggg AAA | | QRANLRAHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDKLVRHX₁₇ | |
| | CAG | | GKSFSRSDKLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QAGHLASH | |
| | | | KCPECGKSFSQAGHLASHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDKLV | |
| | | | GEKPYKCPECGKSFSRSDKLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDH | |
| | | | QRTHTGEKPYKCPECGKSFSRSD | | LTTHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | HLTTHQRTHTGEKPYKCPECGKS | | DKLVRHX₁₇X₁₈X₁₉HX₂₀X₂₁X₂₂X₂₃X₂₄X₂₅X₂₆X₂₇X₂₈X₂₉X₃₀ | |
| | | | FSRSDKLVRHQRTHTGEKPTGKK | | | |
| | | | TS | | | |
| tgagggaaa | TGA ggg | 900 | LEPGEKPYKCPECGKSFSRSDEL | 1449 | X₁X₂X₃X₄X₅X₆X₇X₈X₉CX₁₀X₁₁CX₁₂X₁₃X₁₄X₁₅X₁₆RSDELVRHX₁₇X₁₈X₁₉ | 1998 |
| caggagaat | AAA CAG | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TTGNLTVHX₁₇X₁₈ | |
| gtg | gag AAT | | TTGNLTVHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDNLVRHX₁₇ | |
| | GTG | | GKSFSRSDNLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RADNLTEH | |
| | | | KCPECGKSFSRADNLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QRANLR | |
| | | | GEKPYKCPECGKSFSQRANLRAH | | AHX₁₇X₁₈X₁₉HX₂₀X₂₁X₂₂X₂₃X₂₄X₈X₉CX₁₀X₁₁CX₁₂X₁₃X₁₄X₁₅X₁₆RSDK | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆QA | |
| | | | KLVRHQRTHTGEKPYKCPECGKS | | GHLASHX₁₇X₁₈X₁₉HX₂₀X₂₁X₂₂X₂₃X₂₄X₂₅X₂₆X₂₇X₂₈X₂₉X₃₀ | |
| | | | FSQAGHLASHQRTHTGEKPTGKK | | | |
| | | | TS | | | |
| tgggggtga | TGG GGG | 901 | LEPGEKPYKCPECGKSFSRSDNL | 1450 | X₁X₂X₃X₄X₅X₆X₇X₈X₉CX₁₀X₁₁CX₁₂X₁₃X₁₄X₁₅X₁₆RSDNLVRHX₁₇X₁₈X₁₉ | 1999 |
| gggaaacag | TGA ggg | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RADNLTEHX₁₇X₁₈ | |
| gag | AAA CAG | | RADNLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QRANLRAHX₁₇ | |
| | GAG | | GKSFSQRANLRAHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDKLVRH | |
| | | | KCPECGKSFSRSDKLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QAGHLA | |
| | | | GEKPYKCPECGKSFSQAGHLASH | | SHX₁₇X₁₈X₁₉HX₂₀X₂₁X₂₂X₂₃X₂₄X₈X₉CX₁₀X₁₁CX₁₂X₁₃X₁₄X₁₅X₁₆RSDK | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | KLVRHQRTHTGEKPYKCPECGKS | | DHLTTHX₁₇X₁₈X₁₉HX₂₀X₂₁X₂₂X₂₃X₂₄X₂₅X₂₆X₂₇X₂₈X₂₉X₃₀ | |
| | | | FSRSDHLTTHQRTHTGEKPTGKK | | | |
| | | | TS | | | |
| gggaggggg | ggg AGG | 902 | LEPGEKPYKCPECGKSFSRSDKL | 1451 | X₁X₂X₃X₄X₅X₆X₇X₈X₉CX₁₀X₁₁CX₁₂X₁₃X₁₄X₁₅X₁₆RSDKLVRHX₁₇X₁₈X₁₉ | 2000 |
| tgggggtga | GGG TGG | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QAGHLASHX₁₇X₁₈ | |
| ggg | GGG TGA | | QAGHLASHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDKLVRHX₁₇ | |
| | GGG | | GKSFSRSDKLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDHLTTH | |
| | | | KCPECGKSFSRSDHLTTHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDKLV | |
| | | | GEKPYKCPECGKSFSRSDKLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDH | |
| | | | QRTHTGEKPYKCPECGKSFSRSD | | LTNHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | HLTNHQRTHTGEKPYKCPECGKS | | DKLVRHX₁₇X₁₈X₁₉HX₂₀X₂₁X₂₂X₂₃X₂₄X₂₅X₂₆X₂₇X₂₈X₂₉X₃₀ | |
| | | | FSRSDKLVRHQRTHTGEKPTGKK | | | |
| | | | TS | | | |
| agggggtgg | AGG GGG | 903 | LEPGEKPYKCPECGKSFSQRANL | 1452 | X₁X₂X₃X₄X₅X₆X₇X₈X₉CX₁₀X₁₁CX₁₂X₁₃X₁₄X₁₅X₁₆QRANLRAHX₁₇X₁₈X₁₉ | 2001 |
| gggtgaggg | TGG GGG | | RAHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDKLVRHX₁₇X₁₈ | |
| aaa | TGA ggg | | RSDKLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QAGHLASHX₁₇ | |
| | AAA | | GKSFSQAGHLASHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDKLVRH | |
| | | | KCPECGKSFSRSDKLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDHLT | |
| | | | GEKPYKCPECGKSFSRSDHLTTH | | THX₁₇X₁₈X₁₉HX₂₀X₂₁X₂₂X₂₃X₂₄X₈X₉CX₁₀X₁₁CX₁₂X₁₃X₁₄X₁₅X₁₆RSDK | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | KLVRHQRTHTGEKPYKCPECGKS | | DHLTNHX₁₇X₁₈X₁₉HX₂₀X₂₁X₂₂X₂₃X₂₄X₂₅X₂₆X₂₇X₂₈X₂₉X₃₀ | |
| | | | FSRSDHLTNHQRTHTGEKPTGKK | | | |
| | | | TS | | | |
| gggaaacag | ggg AAA | 904 | LEPGEKPYKCPECGKSFSRRDEL | 1453 | X₁X₂X₃X₄X₅X₆X₇X₈X₉CX₁₀X₁₁CX₁₂X₁₃X₁₄X₁₅X₁₆RRDELNVHX₁₇X₁₈X₁₉ | 2002 |
| gagaatgtg | CAG gag | | NVHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDELVRHX₁₇X₁₈ | |
| atg | AAT gtg | | RSDELVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TTGNLTVHX₁₇ | |
| | ATG | | GKSFSTTGNLTVHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDNLVRH | |
| | | | KCPECGKSFSRSDNLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RADNLT | |
| | | | GEKPYKCPECGKSFSRADNLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆QRAN | |
| | | | QRTHTGEKPYKCPECGKSFSQRA | | LRAHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | NLRAHQRTHTGEKPYKCPECGKS | | DKLVRHX₁₇X₁₈X₁₉HX₂₀X₂₁X₂₂X₂₃X₂₄X₂₅X₂₆X₂₇X₂₈X₂₉X₃₀ | |
| | | | FSRSDKLVRHQRTHTGEKPTGKK | | | |
| | | | TS | | | |
| aaacaggag | AAA CAG | 905 | LEPGEKPYKCPECGKSFSQRAHL | 1454 | X₁X₂X₃X₄X₅X₆X₇X₈X₉CX₁₀X₁₁CX₁₂X₁₃X₁₄X₁₅X₁₆QRAHLERHX₁₇X₁₈X₁₉ | 2003 |
| aatgtgatg | gag AAT | | ERHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RRDELNVHX₁₇X₁₈ | |
| gga | gtg ATG | | RRDELNVHQRTHTGEKPYKCPEC | | X₁₁HX₂₀X₂₁X₂₂X₂₃X₂₄X₈X₉CX₁₀X₁₁CX₁₂X₁₃X₁₄X₁₅X₁₆RSDELVRHX₁₇ | |
| | GGA | | GKSFSRSDELVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TTGNLTVH | |
| | | | KCPECGKSFSTTGNLTVHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDNLV | |
| | | | GEKPYKCPECGKSFSRSDNLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RADN | |
| | | | QRTHTGEKPYKCPECGKSFSRAD | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆QR | |
| | | | NLTEHQRTHTGEKPYKCPECGKS | | ANLRAHX₁₇X₁₈X₁₉HX₂₀X₂₁X₂₂X₂₃X₂₄X₂₅X₂₆X₂₇X₂₈X₂₉X₃₀ | |
| | | | FSQRANLRAHQRTHTGEKPTGKK | | | |
| | | | TS | | | |
| caggagaat | CAG gag | 906 | LEPGEKPYKCPECGKSFSQRANL | 1455 | X₁X₂X₃X₄X₅X₆X₇X₈X₉CX₁₀X₁₁CX₁₂X₁₃X₁₄X₁₅X₁₆QRANLRAHX₁₇X₁₈X₁₉ | 2004 |
| gtgatggga | AAT gtg | | RAHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QRAHLERHX₁₇X₁₈ | |
| aaa | ATG GGA | | QRAHLERHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RRDELNVHX₁₇ | |
| | AAA | | GKSFSRRDELNVHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDELVRH | |
| | | | KCPECGKSFSRSDELVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TTGNLT | |
| | | | GEKPYKCPECGKSFSTTGNLTVH | | VHX₁₇X₁₈X₁₉HX₂₀X₂₁X₂₂X₂₃X₂₄X₈X₉CX₁₀X₁₁CX₁₂X₁₃X₁₄X₁₅X₁₆RSDN | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RA | |
| | | | NLVRHQRTHTGEKPYKCPECGKS | | DNLTEHX₁₇X₁₈X₁₉HX₂₀X₂₁X₂₂X₂₃X₂₄X₂₅X₂₆X₂₇X₂₈X₂₉X₃₀ | |
| | | | FSRADNLTEHQRTHTGEKPTGKK | | | |
| | | | TS | | | |
| ggccagaaa | GGC CAG | 907 | LEPGEKPYKCPECGKSFSRNDAL | 1456 | X₁X₂X₃X₄X₅X₆X₇X₈X₉CX₁₀X₁₁CX₁₂X₁₃X₁₄X₁₅X₁₆RNDALTEHX₁₇X₁₈X₁₉ | 2005 |
| cactgggag | AAA CAC | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDNLVRHX₁₇X₁₈ | |
| ctg | TGG gag | | RSDNLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDHLTTHX₁₇ | |
| | CTG | | GKSFSRSDHLTTHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆SKKALTEH | |
| | | | KCPECGKSFSSKKALTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QRANLR | |
| | | | GEKPYKCPECGKSFSQRANLRAH | | AHX₁₇X₁₈X₁₉HX₂₀X₂₁X₂₂X₂₃X₂₄X₈X₉CX₁₀X₁₁CX₁₂X₁₃X₁₄X₁₅X₁₆RADN | |
| | | | QRTHTGEKPYKCPECGKSFSRAD | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆DP | |
| | | | NLTEHQRTHTGEKPYKCPECGKS | | GHLVRHX₁₇X₁₈X₁₉HX₂₀X₂₁X₂₂X₂₃X₂₄X₂₅X₂₆X₂₇X₂₈X₂₉X₃₀ | |
| | | | FSDPGHLVRHQRTHTGEKPTGKK | | | |
| | | | TS | | | |
| ctgggccag | CTG GGC | 908 | LEPGEKPYKCPECGKSFSRSDNL | 1457 | X₁X₂X₃X₄X₅X₆X₇X₈X₉CX₁₀X₁₁CX₁₂X₁₃X₁₄X₁₅X₁₆RSDNLVRHX₁₇X₁₈X₁₉ | 2006 |
| aaacactgg | CAG AAA | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDHLTTHX₁₇X₁₈ | |
| gag | CAC TGG | | RSDHLTTHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆SKKALTEHX₁₇ | |
| | GAG | | GKSFSSKKALTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QRANLRAH | |
| | | | KCPECGKSFSQRANLRAHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RADNLT | |
| | | | GEKPYKCPECGKSFSRADNLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆DPGH | |
| | | | QRTHTGEKPYKCPECGKSFSDPG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RN | |
| | | | HLVRHQRTHTGEKPYKCPECGKS | | DALTEHX₁₇X₁₈X₁₉HX₂₀X₂₁X₂₂X₂₃X₂₄X₂₅X₂₆X₂₇X₂₈X₂₉X₃₀ | |
| | | | FSRNDALTEHQRTHTGEKPTGKK | | | |
| | | | TS | | | |
| aaacactgg | AAA CAC | 909 | LEPGEKPYKCPECGKSFSRSDNL | 1458 | X₁X₂X₃X₄X₅X₆X₇X₈X₉CX₁₀X₁₁CX₁₂X₁₃X₁₄X₁₅X₁₆RSDNLVRHX₁₇X₁₈X₁₉ | 2007 |
| gagctgtgg | TGG gag | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDHLTTHX₁₇X₁₈ | |
| gag | CTG TGG | | RSDHLTTHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RNDALTEHX₁₇ | |
| | GAG | | GKSFSRNDALTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDNLVRH | |
| | | | KCPECGKSFSRSDNLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDHLT | |
| | | | GEKPYKCPECGKSFSRSDHLTTH | | THX₁₇X₁₈X₁₉HX₂₀X₂₁X₂₂X₂₃X₂₄X₈X₉CX₁₀X₁₁CX₁₂X₁₃X₁₄X₁₅X₁₆SKKA | |
| | | | QRTHTGEKPYKCPECGKSFSSKK | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆QR | |
| | | | ALTEHQRTHTGEKPYKCPECGKS | | ANLRAHX₁₇X₁₈X₁₉HX₂₀X₂₁X₂₂X₂₃X₂₄X₂₅X₂₆X₂₇X₂₈X₂₉X₃₀ | |
| | | | FSQRANLRAHQRTHTGEKPTGKK | | | |
| | | | TS | | | |
| gccagcctg | GCC AGC | 910 | LEPGEKPYKCPECGKSFSSKKAL | 1459 | X₁X₂X₃X₄X₅X₆X₇X₈X₉CX₁₀X₁₁CX₁₂X₁₃X₁₄X₁₅X₁₆SKKALTEHX₁₇X₁₈X₁₉ | 2008 |
| ggccagaaa | CTG GGC | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QRANLRAHX₁₇X₁₈ | |
| cac | CAG AAA | | QRANLRAHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RADNLTEHX₁₇ | |
| | CAC | | GKSFSRADNLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DPGHLVRH | |
| | | | KCPECGKSFSDPGHLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RNDALT | |
| | | | GEKPYKCPECGKSFSRNDALTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆ERSH | |
| | | | QRTHTGEKPYKCPECGKSFSERS | | LREHX₁₇X₁₈X₁₉HX₂₀X₂₁X₂₂X₂₃X₂₄X₈X₉CX₁₀X₁₁CX₁₂X₁₃X₁₄X₁₅X₁₆DC | |
| | | | HLREHQRTHTGEKPYKCPECGKS | | RDLARHX₁₇X₁₈X₁₉HX₂₀X₂₁X₂₂X₂₃X₂₄X₂₅X₂₆X₂₇X₂₈X₂₉X₃₀ | |
| | | | FSDCRDLARHQRTHTGEKPTGKK | | | |
| | | | TS | | | |
| agcctgggc | AGC CTG | 911 | LEPGEKPYKCPECGKSFSRSDHL | 1460 | X₁X₂X₃X₄X₅X₆X₇X₈X₉CX₁₀X₁₁CX₁₂X₁₃X₁₄X₁₅X₁₆RSDHLTTHX₁₇X₁₈X₁₉ | 2009 |
| cagaaacac | GGC CAG | | TTHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆SKKALTEHX₁₇X₁₈ | |
| tgg | AAA CAC | | SKKALTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QRANLRAHX₁₇ | |
| | TGG | | GKSFSQRANLRAHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RADNLTEH | |
| | | | KCPECGKSFSRADNLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DPGHLV | |
| | | | GEKPYKCPECGKSFSDPGHLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RNDA | |
| | | | QRTHTGEKPYKCPECGKSFSRND | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆ER | |
| | | | ALTEHQRTHTGEKPYKCPECGKS | | SHLREHX₁₇X₁₈X₁₉HX₂₀X₂₁X₂₂X₂₃X₂₄X₂₅X₂₆X₂₇X₂₈X₂₉X₃₀ | |
| | | | FSERSHLREHQRTHTGEKPTGKK | | | |
| | | | TS | | | |
| cagaaacac | CAG AAA | 912 | LEPGEKPYKCPECGKSFSRSDHL | 1461 | X₁X₂X₃X₄X₅X₆X₇X₈X₉CX₁₀X₁₁CX₁₂X₁₃X₁₄X₁₅X₁₆RSDHLTTHX₁₇X₁₈X₁₉ | 2010 |
| tgggagctg | CAC TGG | | TTHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RNDALTEHX₁₇X₁₈ | |
| tgg | gag CTG | | RNDALTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDNLVRHX₁₇ | |
| | TGG | | GKSFSRSDNLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDHLTTH | |
| | | | KCPECGKSFSRSDHLTTHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆SKKALT | |
| | | | GEKPYKCPECGKSFSSKKALTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆QRAN | |
| | | | QRTHTGEKPYKCPECGKSFSQRA | | LRAHX₁₇X₁₈X₁₉HX₂₀X₂₁X₂₂X₂₃X₂₄X₈X₉CX₁₀X₁₁CX₁₂X₁₃X₁₄X₁₅X₁₆RA | |
| | | | NLRAHQRTHTGEKPYKCPECGKS | | DNLTEHX₁₇X₁₈X₁₉HX₂₀X₂₁X₂₂X₂₃X₂₄X₂₅X₂₆X₂₇X₂₈X₂₉X₃₀ | |
| | | | FSRADNLTEHQRTHTGEKPTGKK | | | |
| | | | TS | | | |
| gatggagcc | GAT GGA | 913 | LEPGEKPYKCPECGKSFSRADNL | 1462 | X₁X₂X₃X₄X₅X₆X₇X₈X₉CX₁₀X₁₁CX₁₂X₁₃X₁₄X₁₅X₁₆RADNLTEHX₁₇X₁₈X₁₉ | 2011 |
| agcctgggc | GCC AGC | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆DPGHLVRHX₁₇X₁₈ | |
| cag | CTG GGC | | DPGHLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RNDALTEHX₁₇ | |
| | CAG | | GKSFSRNDALTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆ERSHLREH | |
| | | | KCPECGKSFSERSHLREHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DCRDLA | |
| | | | GEKPYKCPECGKSFSDCRDLARH | | RHX₁₇X₁₈X₁₉HX₂₀X₂₁X₂₂X₂₃X₂₄X₈X₉CX₁₀X₁₁CX₁₂X₁₃X₁₄X₁₅X₁₆QRAH | |
| | | | QRTHTGEKPYKCPECGKSFSQRA | | LERHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | HLERHQRTHTGEKPYKCPECGKS | | GNLVRHX₁₇X₁₈X₁₉HX₂₀X₂₁X₂₂X₂₃X₂₄X₂₅X₂₆X₂₇X₂₈X₂₉X₃₀ | |
| | | | FSTSGNLVRHQRTHTGEKPTGKK | | | |
| | | | TS | | | |
| ggagccagc | GGA GCC | 914 | LEPGEKPYKCPECGKSFSQRANL | 1463 | X₁X₂X₃X₄X₅X₆X₇X₈X₉CX₁₀X₁₁CX₁₂X₁₃X₁₄X₁₅X₁₆QRANLRAHX₁₇X₁₈X₁₉ | 2012 |
| ctgggccag | AGC CTG | | RAHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RADNLTEHX₁₇X₁₈ | |
| aaa | GGC CAG | | RADNLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DPGHLVRHX₁₇ | |
| | AAA | | GKSFSDPGHLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RNDALTEH | |
| | | | KCPECGKSFSRNDALTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆ERSHLR | |
| | | | GEKPYKCPECGKSFSERSHLREH | | EHX₁₇X₁₈X₁₉HX₂₀X₂₁X₂₂X₂₃X₂₄X₈X₉CX₁₀X₁₁CX₁₂X₁₃X₁₄X₁₅X₁₆DCRD | |
| | | | QRTHTGEKPYKCPECGKSFSDCR | | LARHX₁₇X₁₈X₁₉HX₂₀X₂₁X₂₂X₂₃X₂₄X₈X₉CX₁₀X₁₁CX₁₂X₁₃X₁₄X₁₅X₁₆QR | |
| | | | DLARHQRTHTGEKPYKCPECGKS | | AHLERHX₁₇X₁₈X₁₉HX₂₀X₂₁X₂₂X₂₃X₂₄X₂₅X₂₆X₂₇X₂₈X₂₉X₃₀ | |
| | | | FSQRAHLERHQRTHTGEKPTGKK | | | |
| | | | TS | | | |
| tgggagctg | TGG gag | 915 | LEPGEKPYKCPECGKSFSRSDNL | 1464 | X₁X₂X₃X₄X₅X₆X₇X₈X₉CX₁₀X₁₁CX₁₂X₁₃X₁₄X₁₅X₁₆RSDNLVRHX₁₇X₁₈X₁₉ | 2013 |
| tgggagacg | CTG TGG | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RTDTLRDHX₁₇X₁₈ | |
| gag | gag ACG | | RTDTLRDHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDNLVRHX₁₇ | |
| | GAG | | GKSFSRSDNLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDHLTTH | |
| | | | KCPECGKSFSRSDHLTTHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RNDALT | |
| | | | GEKPYKCPECGKSFSRNDALTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆RSDN | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | NLVRHQRTHTGEKPYKCPECGKS | | DHLTTHX₁₇X₁₈X₁₉HX₂₀X₂₁X₂₂X₂₃X₂₄X₂₅X₂₆X₂₇X₂₈X₂₉X₃₀ | |
| | | | FSRSDHLTTHQRTHTGEKPTGKK | | | |
| | | | TS | | | |
| gagctgtgg | gag CTG | 916 | LEPGEKPYKCPECGKSFSRSDHL | 1465 | X₁X₂X₃X₄X₅X₆X₇X₈X₉CX₁₀X₁₁CX₁₂X₁₃X₁₄X₁₅X₁₆RSDHLTNHX₁₇X₁₈X₁₉ | 2014 |
| gagacggag | TGG gag | | TNHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDNLVRHX₁₇X₁₈ | |
| agg | ACG gag | | RSDNLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RTDTLRDHX₁₇ | |
| | AGG | | GKSFSRTDTLRDHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDNLVRH | |
| | | | KCPECGKSFSRSDNLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDHLT | |
| | | | GEKPYKCPECGKSFSRSDHLTTH | | THX₁₇X₁₈X₁₉HX₂₀X₂₁X₂₂X₂₃X₂₄X₈X₉CX₁₀X₁₁CX₁₂X₁₃X₁₄X₁₅X₁₆RNDA | |
| | | | QRTHTGEKPYKCPECGKSFSRND | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | ALTEHQRTHTGEKPYKCPECGKS | | DNLVRHX₁₇X₁₈X₁₉HX₂₀X₂₁X₂₂X₂₃X₂₄X₂₅X₂₆X₂₇X₂₈X₂₉X₃₀ | |
| | | | FSRSDNLVRHQRTHTGEKPTGKK | | | |
| | | | TS | | | |
| ctgtgggag | CTG TGG | 917 | LEPGEKPYKCPECGKSFSDPGHL | 1466 | X₁X₂X₃X₄X₅X₆X₇X₈X₉CX₁₀X₁₁CX₁₂X₁₃X₁₄X₁₅X₁₆DPGHLVRHX₁₇X₁₈X₁₉ | 2015 |
| acggagagg | gag ACG | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDHLTNHX₁₇X₁₈ | |
| ggc | gag AGG | | RSDHLTNHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDNLVRHX₁₇ | |
| | GGC | | GKSFSRSDNLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RTDTLRDH | |
| | | | KCPECGKSFSRTDTLRDHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDNLV | |
| | | | GEKPYKCPECGKSFSRSDNLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDH | |
| | | | QRTHTGEKPYKCPECGKSFSRSD | | LTTHX₁₇X₁₈X₁₉HX₂₀X₂₁X₂₂X₂₃X₂₄X₈X₉CX₁₀X₁₁CX₁₂X₁₃X₁₄X₁₅X₁₆RN | |
| | | | HLTTHQRTHTGEKPYKCPECGKS | | DALTEHX₁₇X₁₈X₁₉HX₂₀X₂₁X₂₂X₂₃X₂₄X₂₅X₂₆X₂₇X₂₈X₂₉X₃₀ | |
| | | | FSRNDALTEHQRTHTGEKPTGKK | | | |
| | | | TS | | | |
| gagacggag | gag ACG | 918 | LEPGEKPYKCPECGKSFSRSDEL | 1467 | X₁X₂X₃X₄X₅X₆X₇X₈X₉CX₁₀X₁₁CX₁₂X₁₃X₁₄X₁₅X₁₆RSDELVRHX₁₇X₁₈X₁₉ | 2016 |
| aggggcagg | gag AGG | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDHLTNHX₁₇X₁₈ | |
| gtg | GGC AGG | | RSDHLTNHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DPGHLVRHX₁₇ | |
| | GTG | | GKSFSDPGHLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDHLTNH | |
| | | | KCPECGKSFSRSDHLTNHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDNLV | |
| | | | GEKPYKCPECGKSFSRSDNLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RTDT | |
| | | | QRTHTGEKPYKCPECGKSFSRTD | | LRDHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | TLRDHQRTHTGEKPYKCPECGKS | | DNLVRHX₁₇X₁₈X₁₉HX₂₀X₂₁X₂₂X₂₃X₂₄X₂₅X₂₆X₂₇X₂₈X₂₉X₃₀ | |
| | | | FSRSDNLVRHQRTHTGEKPTGKK | | | |
| | | | TS | | | |
| tgggagacg | TGG gag | 919 | LEPGEKPYKCPECGKSFSRSDHL | 1468 | X₁X₂X₃X₄X₅X₆X₇X₈X₉CX₁₀X₁₁CX₁₂X₁₃X₁₄X₁₅X₁₆RSDHLTNHX₁₇X₁₈X₁₉ | 2017 |
| gagaggggc | ACG gag | | TNHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆DPGHLVRHX₁₇X₁₈ | |
| agg | AGG GGC | | DPGHLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDHLTNHX₁₇ | |
| | AGG | | GKSFSRSDHLTNHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDNLVRH | |
| | | | KCPECGKSFSRSDNLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RTDTLR | |
| | | | GEKPYKCPECGKSFSRTDTLRDH | | DHX₁₇X₁₈X₁₉HX₂₀X₂₁X₂₂X₂₃X₂₄X₈X₉CX₁₀X₁₁CX₁₂X₁₃X₁₄X₁₅X₁₆RSDN | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | NLVRHQRTHTGEKPYKCPECGKS | | DHLTTHX₁₇X₁₈X₁₉HX₂₀X₂₁X₂₂X₂₃X₂₄X₂₅X₂₆X₂₇X₂₈X₂₉X₃₀ | |
| | | | FSRSDHLTTHQRTHTGEKPTGKK | | | |
| | | | TS | | | |
| cactgggag | CAC TGG | 920 | LEPGEKPYKCPECGKSFSRTDTL | 1469 | X₁X₂X₃X₄X₅X₆X₇X₈X₉CX₁₀X₁₁CX₁₂X₁₃X₁₄X₁₅X₁₆RTDTLRDHX₁₇X₁₈X₁₉ | 2018 |
| ctgtgggag | gag CTG | | RDHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDNLVRHX₁₇X₁₈ | |
| acg | TGG gag | | RSDNLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDHLTTHX₁₇ | |
| | ACG | | GKSFSRSDHLTTHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RNDALTEH | |
| | | | KCPECGKSFSRNDALTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDNLV | |
| | | | GEKPYKCPECGKSFSRSDNLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDH | |
| | | | QRTHTGEKPYKCPECGKSFSRSD | | LTTHX₁₇X₁₈X₁₉HX₂₀X₂₁X₂₂X₂₃X₂₄X₈X₉CX₁₀X₁₁CX₁₂X₁₃X₁₄X₁₅X₁₆SK | |
| | | | HLTTHQRTHTGEKPYKCPECGKS | | KALTEHX₁₇X₁₈X₁₉HX₂₀X₂₁X₂₂X₂₃X₂₄X₂₅X₂₆X₂₇X₂₈X₂₉X₃₀ | |
| | | | FSSKKALTEHQRTHTGEKPTGKK | | | |
| | | | TS | | | |
| acggagagg | ACG gag | 921 | LEPGEKPYKCPECGKSFSQRAHL | 1470 | X₁X₂X₃X₄X₅X₆X₇X₈X₉CX₁₀X₁₁CX₁₂X₁₃X₁₄X₁₅X₁₆QRAHLERHX₁₇X₁₈X₁₉ | 2019 |
| ggcagggtg | AGG GGC | | ERHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDELVRHX₁₇X₁₈ | |
| gga | AGG GTG | | RSDELVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDHLTNHX₁₇ | |
| | GGA | | GKSFSRSDHLTNHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DPGHLVRH | |
| | | | KCPECGKSFSDPGHLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDHLT | |
| | | | GEKPYKCPECGKSFSRSDHLTNH | | NHX₁₇X₁₈X₁₉HX₂₀X₂₁X₂₂X₂₃X₂₄X₈X₉CX₁₀X₁₁CX₁₂X₁₃X₁₄X₁₅X₁₆RSDN | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RT | |
| | | | NLVRHQRTHTGEKPYKCPECGKS | | DTLRDHX₁₇X₁₈X₁₉HX₂₀X₂₁X₂₂X₂₃X₂₄X₂₅X₂₆X₂₇X₂₈X₂₉X₃₀ | |
| | | | FSRTDTLRDHQRTHTGEKPTGKK | | | |
| | | | TS | | | |
| cgagatgga | CGA GAT | 922 | LEPGEKPYKCPECGKSFSDPGHL | 1471 | X₁X₂X₃X₄X₅X₆X₇X₈X₉CX₁₀X₁₁CX₁₂X₁₃X₁₄X₁₅X₁₆DPGHLVRHX₁₇X₁₈X₁₉ | 2020 |
| gccagcctg | GGA GCC | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RNDALTEHX₁₇X₁₈ | |
| ggc | AGC CTG | | RNDALTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆ERSHLREHX₁₇ | |
| | GGC | | GKSFSERSHLREHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DCRDLARH | |
| | | | KCPECGKSFSDCRDLARHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QRAHLE | |
| | | | GEKPYKCPECGKSFSQRAHLERH | | RHX₁₇X₁₈X₁₉HX₂₀X₂₁X₂₂X₂₃X₂₄X₈X₉CX₁₀X₁₁CX₁₂X₁₃X₁₄X₁₅X₁₆TSGN | |
| | | | QRTHTGEKPYKCPECGKSFSTSG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆QS | |
| | | | NLVRHQRTHTGEKPYKCPECGKS | | GHLTEHX₁₇X₁₈X₁₉HX₂₀X₂₁X₂₂X₂₃X₂₄X₂₅X₂₆X₂₇X₂₈X₂₉X₃₀ | |
| | | | FSQSGHLTEHQRTHTGEKPTGKK | | | |
| | | | TS | | | |
| gcctgggcc | GCC TGG | 923 | LEPGEKPYKCPECGKSFSRSDKL | 1472 | X₁X₂X₃X₄X₅X₆X₇X₈X₉CX₁₀X₁₁CX₁₂X₁₃X₁₄X₁₅X₁₆RSDKLVRHX₁₇X₁₈X₁₉ | 2021 |
| agaaacact | GCC AGA | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆THLDLIRHX₁₇X₁₈ | |
| ggg | AAC ACT | | THLDLIRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DSGNLRVHX₁₇ | |
| | GGG | | GKSFSDSGNLRVHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QLAHLRAH | |
| | | | KCPECGKSFSQLAHLRAHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DCRDLA | |
| | | | GEKPYKCPECGKSFSDCRDLARH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDH | |
| | | | QRTHTGEKPYKCPECGKSFSRSD | | LTTHX₁₇X₁₈X₁₉HX₂₀X₂₁X₂₂X₂₃X₂₄X₈X₉CX₁₀X₁₁CX₁₂X₁₃X₁₄X₁₅X₁₆DC | |
| | | | HLTTHQRTHTGEKPYKCPECGKS | | RDLARHX₁₇X₁₈X₁₉HX₂₀X₂₁X₂₂X₂₃X₂₄X₂₅X₂₆X₂₇X₂₈X₂₉X₃₀ | |
| | | | FSDCRDLARHQRTHTGEKPTGKK | | | |
| | | | TS | | | |
| cagcctgg | CCA GCC | 924 | LEPGEKPYKCPECGKSFSTHLDL | 1473 | X₁X₂X₃X₄X₅X₆X₇X₈X₉CX₁₀X₁₁CX₁₂X₁₃X₁₄X₁₅X₁₆THLDLIRHX₁₇X₁₈X₁₉ | 2022 |
| gccagaaac | TGG GCC | | IRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆DSGNLRVHX₁₇X₁₈ | |
| act | AGA AAC | | DSGNLRVHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QLAHLRAHX₁₇ | |
| | ACT | | GKSFSQLAHLRAHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DCRDLARH | |
| | | | KCPECGKSFSDCRDLARHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDHLT | |
| | | | GEKPYKCPECGKSFSRSDHLTTH | | THX₁₇X₁₈X₁₉HX₂₀X₂₁X₂₂X₂₃X₂₄X₈X₉CX₁₀X₁₁CX₁₂X₁₃X₁₄X₁₅X₁₆DCRD | |
| | | | QRTHTGEKPYKCPECGKSFSDCR | | LARHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | DLARHQRTHTGEKPYKCPECGKS | | HSLTEHX₁₇X₁₈X₁₉HX₂₀X₂₁X₂₂X₂₃X₂₄X₂₅X₂₆X₂₇X₂₈X₂₉X₃₀ | |
| | | | FSTSHSLTEHQRTHTGEKPTGKK | | | |
| | | | TS | | | |
| tgggccaga | TGG GCC | 925 | LEPGEKPYKCPECGKSFSERSHL | 1474 | X₁X₂X₃X₄X₅X₆X₇X₈X₉CX₁₀X₁₁CX₁₂X₁₃X₁₄X₁₅X₁₆ERSHLREHX₁₇X₁₈X₁₉ | 2023 |
| aacactggg | AGA AAC | | REHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDKLVRHX₁₇X₁₈ | |
| agc | ACT ggg | | RSDKLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆THLDLIRHX₁₇ | |
| | AGC | | GKSFSTHLDLIRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DSGNLRVH | |
| | | | KCPECGKSFSDSGNLRVHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QLAHLR | |
| | | | GEKPYKCPECGKSFSQLAHLRAH | | AHX₁₇X₁₈X₁₉HX₂₀X₂₁X₂₂X₂₃X₂₄X₈X₉CX₁₀X₁₁CX₁₂X₁₃X₁₄X₁₅X₁₆DCRD | |
| | | | QRTHTGEKPYKCPECGKSFSDCR | | LARHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | DLARHQRTHTGEKPYKCPECGKS | | DHLTTHX₁₇X₁₈X₁₉HX₂₀X₂₁X₂₂X₂₃X₂₄X₂₅X₂₆X₂₇X₂₈X₂₉X₃₀ | |
| | | | FSRSDHLTTHQRTHTGEKPTGKK | | | |
| | | | TS | | | |
| gagccagcc | gag CCA | 926 | LEPGEKPYKCPECGKSFSDSGNL | 1475 | X₁X₂X₃X₄X₅X₆X₇X₈X₉CX₁₀X₁₁CX₁₂X₁₃X₁₄X₁₅X₁₆DSGNLRVHX₁₇X₁₈X₁₉ | 2024 |
| tgggccaga | GCC TGG | | RVHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QLAHLRAHX₁₇X₁₈ | |
| aac | GCC AGA | | QLAHLRAHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DCRDLARHX₁₇ | |
| | AAC | | GKSFSDCRDLARHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDHLTTH | |
| | | | KCPECGKSFSRSDHLTTHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DCRDLA | |
| | | | GEKPYKCPECGKSFSDCRDLARH | | RHX₁₇X₁₈X₁₉HX₂₀X₂₁X₂₂X₂₃X₂₄X₈X₉CX₁₀X₁₁CX₁₂X₁₃X₁₄X₁₅X₁₆TSHS | |
| | | | QRTHTGEKPYKCPECGKSFSTSH | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | SLTEHQRTHTGEKPYKCPECGKS | | DNLVRHX₁₇X₁₈X₁₉HX₂₀X₂₁X₂₂X₂₃X₂₄X₂₅X₂₆X₂₇X₂₈X₂₉X₃₀ | |
| | | | FSRSDNLVRHQRTHTGEKPTGKK | | | |
| | | | TS | | | |
| atggagcca | ATG gag | 927 | LEPGEKPYKCPECGKSFSQLAHL | 1476 | X₁X₂X₃X₄X₅X₆X₇X₈X₉CX₁₀X₁₁CX₁₂X₁₃X₁₄X₁₅X₁₆QLAHLRAHX₁₇X₁₈X₁₉ | 2025 |
| gcctgggcc | CCA GCC | | RAHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆DCRDLARHX₁₇X₁₈ | |
| aga | TGG GCC | | DCRDLARHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDHLTTHX₁₇ | |
| | AGA | | GKSFSRSDHLTTHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DCRDLARH | |
| | | | KCPECGKSFSDCRDLARHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSHSLT | |
| | | | GEKPYKCPECGKSFSTSHSLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆RSDN | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RR | |
| | | | NLVRHQRTHTGEKPYKCPECGKS | | DELNVHX₁₇X₁₈X₁₉HX₂₀X₂₁X₂₂X₂₃X₂₄X₂₅X₂₆X₂₇X₂₈X₂₉X₃₀ | |
| | | | FSRRDELNVHQRTHTGEKPTGKK | | | |
| | | | TS | | | |
| gagatggag | gag ATG | 928 | LEPGEKPYKCPECGKSFSDCRDL | 1477 | X₁X₂X₃X₄X₅X₆X₇X₈X₉CX₁₀X₁₁CX₁₂X₁₃X₁₄X₁₅X₁₆DCRDLARHX₁₇X₁₈X₁₉ | 2026 |
| ccagcctgg | gag CCA | | ARHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDHLTTHX₁₇X₁₈ | |
| gcc | GCC TGG | | RSDHLTTHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DCRDLARHX₁₇ | |
| | GCC | | GKSFSDCRDLARHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSHSLTEH | |
| | | | KCPECGKSFSTSHSLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDNLV | |
| | | | GEKPYKCPECGKSFSRSDNLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RRDE | |
| | | | QRTHTGEKPYKCPECGKSFSRRD | | LNVHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | ELNVHQRTHTGEKPYKCPECGKS | | DNLVRHX₁₇X₁₈X₁₉HX₂₀X₂₁X₂₂X₂₃X₂₄X₂₅X₂₆X₂₇X₂₈X₂₉X₃₀ | |
| | | | FSRSDNLVRHQRTHTGEKPTGKK | | | |
| | | | TS | | | |
| gggagacgg | ggg AGA | 929 | LEPGEKPYKCPECGKSFSRSDKL | 1478 | X₁X₂X₃X₄X₅X₆X₇X₈X₉CX₁₀X₁₁CX₁₂X₁₃X₁₄X₁₅X₁₆RSDKLVRHX₁₇X₁₈X₁₉ | 2027 |
| agaggggca | CGG AGA | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QSGDLRRHX₁₇X₁₈ | |
| ggg | GGG GCA | | QSGDLRRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDKLVRHX₁₇ | |
| | GGG | | GKSFSRSDKLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QLAHLRAH | |
| | | | KCPECGKSFSQLAHLRAHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDKLT | |
| | | | GEKPYKCPECGKSFSRSDKLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆QLAH | |
| | | | QRTHTGEKPYKCPECGKSFSQLA | | LRAHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | HLRAHQRTHTGEKPYKCPECGKS | | DKLVRHX₁₇X₁₈X₁₉HX₂₀X₂₁X₂₂X₂₃X₂₄X₂₅X₂₆X₂₇X₂₈X₂₉X₃₀ | |
| | | | FSRSDKLVRHQRTHTGEKPTGKK | | | |
| | | | TS | | | |
| agacggaga | AGA CGG | 930 | LEPGEKPYKCPECGKSFSRSDHL | 1479 | X₁X₂X₃X₄X₅X₆X₇X₈X₉CX₁₀X₁₁CX₁₂X₁₃X₁₄X₁₅X₁₆RSDHLTTHX₁₇X₁₈X₁₉ | 2028 |
| ggggcaggg | AGA GGG | | TTHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDKLVRHX₁₇X₁₈ | |
| tgg | GCA GGG | | RSDKLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QSGDLRRHX₁₇ | |
| | TGG | | GKSF SQSGDLRRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDKLVRH | |
| | | | KCPECGKSFSRSDKLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QLAHLR | |
| | | | GEKPYKCPECGKSFSQLAHLRAH | | AHX₁₇X₁₈X₁₉HX₂₀X₂₁X₂₂X₂₃X₂₄X₈X₉CX₁₀X₁₁CX₁₂X₁₃X₁₄X₁₅X₁₆RSDK | |
| | | | QRTHTGEKPYKCPECGKSFSRSD | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆QL | |
| | | | KLTEHQRTHTGEKPYKCPECGKS | | AHLRAHX₁₇X₁₈X₁₉HX₂₀X₂₁X₂₂X₂₃X₂₄X₂₅X₂₆X₂₇X₂₈X₂₉X₃₀ | |
| | | | FSQLAHLRAHQRTHTGEKPTGKK | | | |
| | | | TS | | | |
| cggagaggg | CGG AGA | 931 | LEPGEKPYKCPECGKSFSTSGNL | 1480 | X₁X₂X₃X₄X₅X₆X₇X₈X₉CX₁₀X₁₁CX₁₂X₁₃X₁₄X₁₅X₁₆TSGNLVRHX₁₇X₁₈X₁₉ | 2029 |
| gcagggtgg | GGG GCA | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDHLTTHX₁₇X₁₈ | |
| gat | GGG TGG | | RSDHLTTHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDKLVRHX₁₇ | |
| | GAT | | GKSFSRSDKLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QSGDLRRH | |
| | | | KCPECGKSFSQSGDLRRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDKLV | |
| | | | GEKPYKCPECGKSFSRSDKLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆QLAH | |
| | | | QRTHTGEKPYKCPECGKSFSQLA | | LRAHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | HLRAHQRTHTGEKPYKCPECGKS | | DKLTEHX₁₇X₁₈X₁₉HX₂₀X₂₁X₂₂X₂₃X₂₄X₂₅X₂₆X₂₇X₂₈X₂₉X₃₀ | |
| | | | FSRSDKLTEHQRTHTGEKPTGKK | | | |
| | | | TS | | | |
| agaggggca | AGA GGG | 932 | LEPGEKPYKCPECGKSFSSKKAL | 1481 | X₁X₂X₃X₄X₅X₆X₇X₈X₉CX₁₀X₁₁CX₁₂X₁₃X₁₄X₁₅X₁₆SKKALTEHX₁₇X₁₈X₁₉ | 2030 |
| gggtgggat | GCA GGG | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSGNLVRHX₁₇X₁₈ | |
| cac | TGG GAT | | TSGNLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDHLTTHX₁₇ | |
| | CAC | | GKSFSRSDHLTTHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDKLVRH | |
| | | | KCPECGKSFSRSDKLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QSGDLR | |
| | | | GEKPYKCPECGKSFSQSGDLRRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDK | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆QL | |
| | | | KLVRHQRTHTGEKPYKCPECGKS | | AHLRAHX₁₇X₁₈X₁₉HX₂₀X₂₁X₂₂X₂₃X₂₄X₂₅X₂₆X₂₇X₂₈X₂₉X₃₀ | |
| | | | FSQLAHLRAHQRTHTGEKPTGKK | | | |
| | | | TS | | | |
| gatcacagg | GAT CAC | 933 | LEPGEKPYKCPECGKSFSRSDKL | 1482 | X₁X₂X₃X₄X₅X₆X₇X₈X₉CX₁₀X₁₁CX₁₂X₁₃X₁₄X₁₅X₁₆RSDKLTEHX₁₇X₁₈X₁₉ | 2031 |
| gagcaggag | AGG gag | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDNLVRHX₁₇X₁₈ | |
| cgg | CAG gag | | RSDNLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RADNLTEHX₁₇ | |
| | CGG | | GKSFSRADNLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDNLVRH | |
| | | | KCPECGKSFSRSDNLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDHLT | |
| | | | GEKPYKCPECGKSFSRSDHLTNH | | NHX₁₇X₁₈X₁₉HX₂₀X₂₁X₂₂X₂₃X₂₄X₈X₉CX₁₀X₁₁CX₁₂X₁₃X₁₄X₁₅X₁₆SKKA | |
| | | | QRTHTGEKPYKCPECGKSFSSKK | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | ALTEHQRTHTGEKPYKCPECGKS | | GNLVRHX₁₇X₁₈X₁₉HX₂₀X₂₁X₂₂X₂₃X₂₄X₂₅X₂₆X₂₇X₂₈X₂₉X₃₀ | |
| | | | FSTSGNLVRHQRTHTGEKPTGKK | | | |
| | | | TS | | | |
| ggggcaggg | GGG GCA | 934 | LEPGEKPYKCPECGKSFSRSDHL | 1483 | X₁X₂X₃X₄X₅X₆X₇X₈X₉CX₁₀X₁₁CX₁₂X₁₃X₁₄X₁₅X₁₆RSDHLTNHX₁₇X₁₈X₁₉ | 2032 |
| tgggatcac | GGG TGG | | TNHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆SKKALTEHX₁₇X₁₈ | |
| agg | GAT CAC | | SKKALTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSGNLVRHX₁₇ | |
| | AGG | | GKSFSTSGNLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDHLTTH | |
| | | | KCPECGKSFSRSDHLTTHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDKLV | |
| | | | GEKPYKCPECGKSFSRSDKLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆QSGD | |
| | | | QRTHTGEKPYKCPECGKSFSQSG | | LRRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | DLRRHQRTHTGEKPYKCPECGKS | | DKLVRHX₁₇X₁₈X₁₉HX₂₀X₂₁X₂₂X₂₃X₂₄X₂₅X₂₆X₂₇X₂₈X₂₉X₃₀ | |
| | | | FSRSDKLVRHQRTHTGEKPTGKK | | | |
| | | | TS | | | |
| gggtgggat | GGG TGG | 935 | LEPGEKPYKCPECGKSFSRADNL | 1484 | X₁X₂X₃X₄X₅X₆X₇X₈X₉CX₁₀X₁₁CX₁₂X₁₃X₁₄X₁₅X₁₆RADNLTEHX₁₇X₁₈X₁₉ | 2033 |
| cacagggag | GAT CAC | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDNLVRHX₁₇X₁₈ | |
| cag | AGG gag | | RSDNLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDHLTNHX₁₇ | |
| | CAG | | GKSFSRSDHLTNHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆SKKALTEH | |
| | | | KCPECGKSFSSKKALTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSGNLV | |
| | | | GEKPYKCPECGKSFSTSGNLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDH | |
| | | | QRTHTGEKPYKCPECGKSFSRSD | | LTTHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | HLTTHQRTHTGEKPYKCPECGKS | | DKLVRHX₁₇X₁₈X₁₉HX₂₀X₂₁X₂₂X₂₃X₂₄X₂₅X₂₆X₂₇X₂₈X₂₉X₃₀ | |
| | | | FSRSDKLVRHQRTHTGEKPTGKK | | | |
| | | | TS | | | |
| tgggatcac | TGG GAT | 936 | LEPGEKPYKCPECGKSFSRSDNL | 1485 | X₁X₂X₃X₄X₅X₆X₇X₈X₉CX₁₀X₁₁CX₁₂X₁₃X₁₄X₁₅X₁₆RSDNLVRHX₁₇X₁₈X₁₉ | 2034 |
| agggagcag | CAC AGG | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RADNLTEHX₁₇X₁₈ | |
| gag | gag CAG | | RADNLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDNLVRHX₁₇ | |
| | GAG | | GKSFSRSDNLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDHLTNH | |
| | | | KCPECGKSFSRSDHLTNHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆SKKALT | |
| | | | GEKPYKCPECGKSFSSKKALTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆TSGN | |
| | | | QRTHTGEKPYKCPECGKSFSTSG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | NLVRHQRTHTGEKPYKCPECGKS | | DHLTTHX₁₇X₁₈X₁₉HX₂₀X₂₁X₂₂X₂₃X₂₄X₂₅X₂₆X₂₇X₂₈X₂₉X₃₀ | |
| | | | FSRSDHLTTHQRTHTGEKPTGKK | | | |
| | | | TS | | | |
| gcagggtgg | GCA GGG | 937 | LEPGEKPYKCPECGKSFSRSDNL | 1486 | X₁X₂X₃X₄X₅X₆X₇X₈X₉CX₁₀X₁₁CX₁₂X₁₃X₁₄X₁₅X₁₆RSDNLVRHX₁₇X₁₈X₁₉ | 2035 |
| gatcacagg | TGG GAT | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDHLTNHX₁₇X₁₈ | |
| gag | CAC AGG | | RSDHLTNHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆SKKALTEHX₁₇ | |
| | GAG | | GKSFSSKKALTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSGNLVRH | |
| | | | KCPECGKSFSTSGNLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDHLT | |
| | | | GEKPYKCPECGKSFSRSDHLTTH | | THX₁₇X₁₈X₁₉HX₂₀X₂₁X₂₂X₂₃X₂₄X₈X₉CX₁₀X₁₁CX₁₂X₁₃X₁₄X₁₅X₁₆RSDK | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆QS | |
| | | | KLVRHQRTHTGEKPYKCPECGKS | | GDLRRHX₁₇X₁₈X₁₉HX₂₀X₂₁X₂₂X₂₃X₂₄X₂₅X₂₆X₂₇X₂₈X₂₉X₃₀ | |
| | | | FSQSGDLRRHQRTHTGEKPTGKK | | | |
| | | | TS | | | |
| agggagcag | AGG gag | 938 | LEPGEKPYKCPECGKSFSHKNAL | 1487 | X₁X₂X₃X₄X₅X₆X₇X₈X₉CX₁₀X₁₁CX₁₂X₁₃X₁₄X₁₅X₁₆HKNALQNHX₁₇X₁₈X₁₉ | 2036 |
| gagcgggga | CAG gag | | QNHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QRAHLERHX₁₇X₁₈ | |
| att | CGG GGA | | QRAHLERHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDKLTEHX₁₇ | |
| | ATT | | GKSFSRSDKLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDNLVRH | |
| | | | KCPECGKSFSRSDNLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RADNLT | |
| | | | GEKPYKCPECGKSFSRADNLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆RSDN | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | NLVRHQRTHTGEKPYKCPECGKS | | DHLTNHX₁₇X₁₈X₁₉HX₂₀X₂₁X₂₂X₂₃X₂₄X₂₅X₂₆X₂₇X₂₈X₂₉X₃₀ | |
| | | | FSRSDHLTNHQRTHTGEKPTGKK | | | |
| | | | TS | | | |
| cacagggag | CAC AGG | 939 | LEPGEKPYKCPECGKSFSQRAHL | 1488 | X₁X₂X₃X₄X₅X₆X₇X₈X₉CX₁₀X₁₁CX₁₂X₁₃X₁₄X₁₅X₁₆QRAHLERHX₁₇X₁₈X₁₉ | 2037 |
| caggagcgg | gag CAG | | ERHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDKLTEHX₁₇X₁₈ | |
| gga | gag CGG | | RSDKLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDNLVRHX₁₇ | |
| | GGA | | GKSFSRSDNLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RADNLTEH | |
| | | | KCPECGKSFSRADNLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDNLV | |
| | | | GEKPYKCPECGKSFSRSDNLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDH | |
| | | | QRTHTGEKPYKCPECGKSFSRSD | | LTNHX₁₇X₁₈X₁₉HX₂₀X₂₁X₂₂X₂₃X₂₄X₈X₉CX₁₀X₁₁CX₁₂X₁₃X₁₄X₁₅X₁₆SK | |
| | | | HLTNHQRTHTGEKPYKCPECGKS | | KALTEHX₁₇X₁₈X₁₉HX₂₀X₂₁X₂₂X₂₃X₂₄X₂₅X₂₆X₂₇X₂₈X₂₉X₃₀ | |
| | | | FSSKKALTEHQRTHTGEKPTGKK | | | |
| | | | TS | | | |
| gagcaggag | gag CAG | 940 | LEPGEKPYKCPECGKSFSQRAHL | 1489 | X₁X₂X₃X₄X₅X₆X₇X₈X₉CX₁₀X₁₁CX₁₂X₁₃X₁₄X₁₅X₁₆QRAHLERHX₁₇X₁₈X₁₉ | 2038 |
| cggggaatt | gag CGG | | ERHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆HKNALQNHX₁₇X₁₈ | |
| gga | GGA ATT | | HKNALQNHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QRAHLERHX₁₇ | |
| | GGA | | GKSFSQRAHLERHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDKLTEH | |
| | | | KCPECGKSFSRSDKLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDNLV | |
| | | | GEKPYKCPECGKSFSRSDNLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RADN | |
| | | | QRTHTGEKPYKCPECGKSFSRAD | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | NLTEHQRTHTGEKPYKCPECGKS | | DNLVRHX₁₇X₁₈X₁₉HX₂₀X₂₁X₂₂X₂₃X₂₄X₂₅X₂₆X₂₇X₂₈X₂₉X₃₀ | |
| | | | FSRSDNLVRHQRTHTGEKPTGKK | | | |
| | | | TS | | | |
| caggagcgg | CAG gag | 941 | LEPGEKPYKCPECGKSFSTSGHL | 1490 | X₁X₂X₃X₄X₅X₆X₇X₈X₉CX₁₀X₁₁CX₁₂X₁₃X₁₄X₁₅X₁₆TSGHLVRHX₁₇X₁₈X₁₉ | 2039 |
| ggaattgga | CGG GGA | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QRAHLERHX₁₇X₁₈ | |
| ggt | ATT GGA | | QRAHLERHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆HKNALQNHX₁₇ | |
| | GGT | | GKSFSHKNALQNHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QRAHLERH | |
| | | | KCPECGKSFSQRAHLERHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDKLT | |
| | | | GEKPYKCPECGKSFSRSDKLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆RSDN | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RA | |
| | | | NLVRHQRTHTGEKPYKCPECGKS | | DNLTEHX₁₇X₁₈X₁₉HX₂₀X₂₁X₂₂X₂₃X₂₄X₂₅X₂₆X₂₇X₂₈X₂₉X₃₀ | |
| | | | FSRADNLTEHQRTHTGEKPTGKK | | | |
| | | | TS | | | |
| gagcgggga | gag CGG | 942 | LEPGEKPYKCPECGKSFSQSSNL | 1491 | X₁X₂X₃X₄X₅X₆X₇X₈X₉CX₁₀X₁₁CX₁₂X₁₃X₁₄X₁₅X₁₆QSSNLVRHX₁₇X₁₈X₁₉ | 2040 |
| attggaggt | GGA ATT | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSGHLVRHX₁₇X₁₈ | |
| gaa | GGA GGT | | TSGHLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QRAHLERHX₁₇ | |
| | GAA | | GKSFSQRAHLERHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆HKNALQNH | |
| | | | KCPECGKSFSHKNALQNHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QRAHLE | |
| | | | GEKPYKCPECGKSFSQRAHLERH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDK | |
| | | | QRTHTGEKPYKCPECGKSFSRSD | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | KLTEHQRTHTGEKPYKCPECGKS | | DNLVRHX₁₇X₁₈X₁₉HX₂₀X₂₁X₂₂X₂₃X₂₄X₂₅X₂₆X₂₇X₂₈X₂₉X₃₀ | |
| | | | FSRSDNLVRHQRTHTGEKPTGKK | | | |
| | | | TS | | | |
| acagggagc | ACA ggg | 943 | LEPGEKPYKCPECGKSFSQSSNL | 1492 | X₁X₂X₃X₄X₅X₆X₇X₈X₉CX₁₀X₁₁CX₁₂X₁₃X₁₄X₁₅X₁₆QSSNLVRHX₁₇X₁₈X₁₉ | 2041 |
| aggagcggg | AGC AGG | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDKLVRHX₁₇X₁₈ | |
| gaa | AGC GGG | | RSDKLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆ERSHLREHX₁₇ | |
| | GAA | | GKSFSERSHLREHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDHLTNH | |
| | | | KCPECGKSFSRSDHLTNHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆ERSHLR | |
| | | | GEKPYKCPECGKSFSERSHLREH | | EHX₁₇X₁₈X₁₉HX₂₀X₂₁X₂₂X₂₃X₂₄X₈X₉CX₁₀X₁₁CX₁₂X₁₃X₁₄X₁₅X₁₆RSDK | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆SP | |
| | | | KLVRHQRTHTGEKPYKCPECGKS | | ADLTRHX₁₇X₁₈X₁₉HX₂₀X₂₁X₂₂X₂₃X₂₄X₂₅X₂₆X₂₇X₂₈X₂₉X₃₀ | |
| | | | FSSPADLTRHQRTHTGEKPTGKK | | | |
| | | | TS | | | |
| gcaggagcg | GCA GGA | 944 | LEPGEKPYKCPECGKSFSRSDHL | 1493 | X₁X₂X₃X₄X₅X₆X₇X₈X₉CX₁₀X₁₁CX₁₂X₁₃X₁₄X₁₅X₁₆RSDHLTNHX₁₇X₁₈X₁₉ | 2042 |
| gggaattgg | GCG ggg | | TNHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDHLTTHX₁₇X₁₈ | |
| agg | AAT TGG | | RSDHLTTHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TTGNLTVHX₁₇ | |
| | AGG | | GKSFSTTGNLTVHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDKLVRH | |
| | | | KCPECGKSFSRSDKLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDDLV | |
| | | | GEKPYKCPECGKSFSRSDDLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆QRAH | |
| | | | QRTHTGEKPYKCPECGKSFSQRA | | LERHX₁₇X₁₈X₁₉HX₂₀X₂₁X₂₂X₂₃X₂₄X₈X₉CX₁₀X₁₁CX₁₂X₁₃X₁₄X₁₅X₁₆QS | |
| | | | HLERHQRTHTGEKPYKCPECGKS | | GDLRRHX₁₇X₁₈X₁₉HX₂₀X₂₁X₂₂X₂₃X₂₄X₂₅X₂₆X₂₇X₂₈X₂₉X₃₀ | |
| | | | FSQSGDLRRHQRTHTGEKPTGKK | | | |
| | | | TS | | | |
| ggagcgggg | GGA GCG | 945 | LEPGEKPYKCPECGKSFSQAGHL | 1494 | X₁X₂X₃X₄X₅X₆X₇X₈X₉CX₁₀X₁₁CX₁₂X₁₃X₁₄X₁₅X₁₆QAGHLASHX₁₇X₁₈X₁₉ | 2043 |
| aattggagg | ggg AAT | | ASHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDHLTNHX₁₇X₁₈ | |
| tga | TGG AGG | | RSDHLTNHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDHLTTHX₁₇ | |
| | TGA | | GKSFSRSDHLTTHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TTGNLTVH | |
| | | | KCPECGKSFSTTGNLTVHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDKLV | |
| | | | GEKPYKCPECGKSFSRSDKLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDD | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆QR | |
| | | | DLVRHQRTHTGEKPYKCPECGKS | | AHLERHX₁₇X₁₈X₁₉HX₂₀X₂₁X₂₂X₂₃X₂₄X₂₅X₂₆X₂₇X₂₈X₂₉X₃₀ | |
| | | | FSQRAHLERHQRTHTGEKPTGKK | | | |
| | | | TS | | | |
| cagggagca | CAG GGA | 946 | LEPGEKPYKCPECGKSFSTTGNL | 1495 | X₁X₂X₃X₄X₅X₆X₇X₈X₉CX₁₀X₁₁CX₁₂X₁₃X₁₄X₁₅X₁₆TTGNLTVHX₁₇X₁₈X₁₉ | 2044 |
| ggagcgggg | GCA GGA | | TVHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDKLVRHX₁₇X₁₈ | |
| aat | GCG ggg | | RSDKLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDDLVRHX₁₇ | |
| | AAT | | GKSFSRSDDLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QRAHLERH | |
| | | | KCPECGKSFSQRAHLERHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QSGDLR | |
| | | | GEKPYKCPECGKSFSQSGDLRRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆QRAH | |
| | | | QRTHTGEKPYKCPECGKSFSQRA | | LERHX₁₇X₁₈X₁₉HX₂₀X₂₁X₂₂X₂₃X₂₄X₈X₉CX₁₀X₁₁CX₁₂X₁₃X₁₄X₁₅X₁₆RA | |
| | | | HLERHQRTHTGEKPYKCPECGKS | | DNLTEHX₁₇X₁₈X₁₉HX₂₀X₂₁X₂₂X₂₃X₂₄X₂₅X₂₆X₂₇X₂₈X₂₉X₃₀ | |
| | | | FSRADNLTEHQRTHTGEKPTGKK | | | |
| | | | TS | | | |
| ggagcagga | GGA GCA | 947 | LEPGEKPYKCPECGKSFSRSDHL | 1496 | X₁X₂X₃X₄X₅X₆X₇X₈X₉CX₁₀X₁₁CX₁₂X₁₃X₁₄X₁₅X₁₆RSDHLTTHX₁₇X₁₈X₁₉ | 2045 |
| gcggggaat | GGA GCG | | TTHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TTGNLTVHX₁₇X₁₈ | |
| tgg | ggg AAT | | TTGNLTVHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDKLVRHX₁₇ | |
| | TGG | | GKSFSRSDKLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDDLVRH | |
| | | | KCPECGKSFSRSDDLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QRAHLE | |
| | | | GEKPYKCPECGKSFSQRAHLERH | | RHX₁₇X₁₈X₁₉HX₂₀X₂₁X₂₂X₂₃X₂₄X₈X₉CX₁₀X₁₁CX₁₂X₁₃X₁₄X₁₅X₁₆QSGD | |
| | | | QRTHTGEKPYKCPECGKSFSQSG | | LRRHX₁₇X₁₈X₁₉HX₂₀X₂₁X₂₂X₂₃X₂₄X₈X₉CX₁₀X₁₁CX₁₂X₁₃X₁₄X₁₅X₁₆QR | |
| | | | DLRRHQRTHTGEKPYKCPECGKS | | AHLERHX₁₇X₁₈X₁₉HX₂₀X₂₁X₂₂X₂₃X₂₄X₂₅X₂₆X₂₇X₂₈X₂₉X₃₀ | |
| | | | FSQRAHLERHQRTHTGEKPTGKK | | | |
| | | | TS | | | |
| ggccctccc | GGC CCT | 948 | LEPGEKPYKCPECGKSFSDPGAL | 1497 | X₁X₂X₃X₄X₅X₆X₇X₈X₉CX₁₀X₁₁CX₁₂X₁₃X₁₄X₁₅X₁₆DPGALVRHX₁₇X₁₈X₁₉ | 2046 |
| aaacttcca | CCC AAA | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSHSLTEHX₁₇X₁₈ | |
| gtc | CTT CCA | | TSHSLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TTGALTEHX₁₇ | |
| | GTC | | GKSFSTTGALTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QRANLRAH | |
| | | | KCPECGKSFSQRANLRAHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆SKKHLA | |
| | | | GEKPYKCPECGKSFSSKKHLAEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆TKNS | |
| | | | QRTHTGEKPYKCPECGKSFSTKN | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆DP | |
| | | | SLTEHQRTHTGEKPYKCPECGKS | | GHLVRHX₁₇X₁₈X₁₉HX₂₀X₂₁X₂₂X₂₃X₂₄X₂₅X₂₆X₂₇X₂₈X₂₉X₃₀ | |
| | | | FSDPGHLVRHQRTHTGEKPTGKK | | | |
| | | | TS | | | |
| cctcccaaa | CCT CCC | 949 | LEPGEKPYKCPECGKSFSTSGNL | 1498 | X₁X₂X₃X₄X₅X₆X₇X₈X₉CX₁₀X₁₁CX₁₂X₁₃X₁₄X₁₅X₁₆TSGNLTEHX₁₇X₁₈X₁₉ | 2047 |
| cttccagtc | AAA CTT | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆DPGALVRHX₁₇X₁₈ | |
| cat | CCA GTC | | DPGALVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSHSLTEHX₁₇ | |
| | CAT | | GKSFSTSHSLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TTGALTEH | |
| | | | KCPECGKSFSTTGALTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QRANLR | |
| | | | GEKPYKCPECGKSFSQRANLRAH | | AHX₁₇X₁₈X₁₉HX₂₀X₂₁X₂₂X₂₃X₂₄X₈X₉CX₁₀X₁₁CX₁₂X₁₃X₁₄X₁₅X₁₆SKKH | |
| | | | QRTHTGEKPYKCPECGKSFSSKK | | LAEHX₁₇X₁₈X₁₉HX₂₀X₂₁X₂₂X₂₃X₂₄X₈X₉CX₁₀X₁₁CX₁₂X₁₃X₁₄X₁₅X₁₆TK | |
| | | | HLAEHQRTHTGEKPYKCPECGKS | | NSLTEHX₁₇X₁₈X₁₉HX₂₀X₂₁X₂₂X₂₃X₂₄X₂₅X₂₆X₂₇X₂₈X₂₉X₃₀ | |
| | | | FSTKNSLTEHQRTHTGEKPTGKK | | | |
| | | | TS | | | |
| gggaaactg | ggg AAA | 950 | LEPGEKPYKCPECGKSFSRSDHL | 1499 | X₁X₂X₃X₄X₅X₆X₇X₈X₉CX₁₀X₁₁CX₁₂X₁₃X₁₄X₁₅X₁₆RSDHLTTHX₁₇X₁₈X₁₉ | 2048 |
| cgggggaac | CTG CGG | | TTHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆DSGNLRVHX₁₇X₁₈ | |
| tgg | ggg AAC | | DSGNLRVHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDKLVRHX₁₇ | |
| | TGG | | GKSFSRSDKLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDKLTEH | |
| | | | KCPECGKSFSRSDKLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RNDALT | |
| | | | GEKPYKCPECGKSFSRNDALTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆QRAN | |
| | | | QRTHTGEKPYKCPECGKSFSQRA | | LRAHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | NLRAHQRTHTGEKPYKCPECGKS | | DKLVRHX₁₇X₁₈X₁₉HX₂₀X₂₁X₂₂X₂₃X₂₄X₂₅X₂₆X₂₇X₂₈X₂₉X₃₀ | |
| | | | FSRSDKLVRHQRTHTGEKPTGKK | | | |
| | | | TS | | | |
| accgggaaa | ACC ggg | 951 | LEPGEKPYKCPECGKSFSDSGNL | 1500 | X₁X₂X₃X₄X₅X₆X₇X₈X₉CX₁₀X₁₁CX₁₂X₁₃X₁₄X₁₅X₁₆DSGNLRVHX₁₇X₁₈X₁₉ | 2049 |
| ctgcggggg | AAA CTG | | RVHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDKLVRHX₁₇X₁₈ | |
| aac | CGG ggg | | RSDKLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDKLTEHX₁₇ | |
| | AAC | | GKSFSRSDKLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RNDALTEH | |
| | | | KCPECGKSFSRNDALTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QRANLR | |
| | | | GEKPYKCPECGKSFSQRANLRAH | | AHX₁₇X₁₈X₁₉HX₂₀X₂₁X₂₂X₂₃X₂₄X₈X₉CX₁₀X₁₁CX₁₂X₁₃X₁₄X₁₅X₁₆RSDK | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂XEX₁₄X₁₅X₁₆DK | |
| | | | KLVRHQRTHTGEKPYKCPECGKS | | KDLTRHX₁₇X₁₈X₁₉HX₂₀X₂₁X₂₂X₂₃X₂₄X₂₅X₂₆X₂₇X₂₈X₂₉X₃₀ | |
| | | | FSDKKDLTRHQRTHTGEKPTGKK | | | |
| | | | TS | | | |
| aaactgcgg | AAA CTG | 952 | LEPGEKPYKCPECGKSFSRKDNL | 1501 | X₁X₂X₃X₄X₅X₆X₇X₈X₉CX₁₀X₁₁CX₁₂X₁₃X₁₄X₁₅X₁₆RKDNLKNHX₁₇X₁₈X₁₉ | 2050 |
| gggaactgg | CGG ggg | | KNHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDHLTTHX₁₇X₁₈ | |
| aag | AAC TGG | | RSDHLTTHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DSGNLRVHX₁₇ | |
| | AAG | | GKSFSDSGNLRVHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDKLVRH | |
| | | | KCPECGKSFSRSDKLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDKLT | |
| | | | GEKPYKCPECGKSFSRSDKLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆RNDA | |
| | | | QRTHTGEKPYKCPECGKSFSRND | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆QR | |
| | | | ALTEHQRTHTGEKPYKCPECGKS | | ANLRAHX₁₇X₁₈X₁₉HX₂₀X₂₁X₂₂X₂₃X₂₄X₂₅X₂₆X₂₇X₂₈X₂₉X₃₀ | |
| | | | FSQRANLRAHQRTHTGEKPTGKK | | | |
| | | | TS | | | |
| ggaaccggg | GGA ACC | 953 | LEPGEKPYKCPECGKSFSRSDKL | 1502 | X₁X₂X₃X₄X₅X₆X₇X₈X₉CX₁₀X₁₁CX₁₂X₁₃X₁₄X₁₅X₁₆RSDKLVRHX₁₇X₁₈X₁₉ | 2051 |
| aaactgcgg | ggg AAA | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDKLTEHX₁₇X₁₈ | |
| ggg | CTG CGG | | RSDKLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RNDALTEHX₁₇ | |
| | GGG | | GKSFSRNDALTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QRANLRAH | |
| | | | KCPECGKSFSQRANLRAHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDKLV | |
| | | | GEKPYKCPECGKSFSRSDKLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆DKKD | |
| | | | QRTHTGEKPYKCPECGKSFSDKK | | LTRHX₁₇X₁₈X₁₉HX₂₀X₂₁X₂₂X₂₃X₂₄X₈X₉CX₁₀X₁₁CX₁₂X₁₃X₁₄X₁₅X₁₆QR | |
| | | | DLTRHQRTHTGEKPYKCPECGKS | | AHLERHX₁₇X₁₈X₁₉HX₂₀X₂₁X₂₂X₂₃X₂₄X₂₅X₂₆X₂₇X₂₈X₂₉X₃₀ | |
| | | | FSQRAHLERHQRTHTGEKPTGKK | | | |
| | | | TS | | | |
| aggggaacc | AGG GGA | 954 | LEPGEKPYKCPECGKSFSRSDKL | 1503 | X₁X₂X₃X₄X₅X₆X₇X₈X₉CX₁₀X₁₁CX₁₂X₁₃X₁₄X₁₅X₁₆RSDKLTEHX₁₇X₁₈X₁₉ | 2052 |
| gggaaactg | ACC ggg | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RNDALTEHX₁₇X₁₈ | |
| cgg | AAA CTG | | RNDALTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QRANLRAHX₁₇ | |
| | CGG | | GKSFSQRANLRAHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDKLVRH | |
| | | | KCPECGKSFSRSDKLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DKKDLT | |
| | | | GEKPYKCPECGKSFSDKKDLTRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆QRAH | |
| | | | QRTHTGEKPYKCPECGKSFSQRA | | LERHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | HLERHQRTHTGEKPYKCPECGKS | | DHLTNHX₁₇X₁₈X₁₉HX₂₀X₂₁X₂₂X₂₃X₂₄X₂₅X₂₆X₂₇X₂₈X₂₉X₃₀ | |
| | | | FSRSDHLTNHQRTHTGEKPTGKK | | | |
| | | | TS | | | |
| ctgcggggg | CTG CGG | 955 | LEPGEKPYKCPECGKSFSQRAHL | 1504 | X₁X₂X₃X₄X₅X₆X₇X₈X₉CX₁₀X₁₁CX₁₂X₁₃X₁₄X₁₅X₁₆QPAHLERHX₁₇X₁₈X₁₉ | 2053 |
| aactggaag | ggg AAC | | ERHQRTHTGEKPYKCPECGKSFS | | HX₂₀X₂₁X₂₂X₂₃X₂₄X₈X₉CX₁₀X₁₁CX₁₂X₁X₁₄X₁₅X₁₆RKDNLKNHX₁₇X₁₈ | |
| gga | TGG AAG | | RKDNLKNHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDHLTTHX₁₇ | |
| | GGA | | GKSFSRSDHLTTHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DSGNLRVH | |
| | | | KCPECGKSFSDSGNLRVHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDKLV | |
| | | | GEKPYKCPECGKSFSRSDKLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDK | |
| | | | QRTHTGEKPYKCPECGKSFSRSD | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆RN | |
| | | | KLTEHQRTHTGEKPYKCPECGKS | | DALTEHX₁₇X₁₈X₁₉HX₂₀X₂₁X₂₂X₂₃X₂₄X₂₅X₂₆X₂₇X₂₈X₂₉X₃₀ | |
| | | | FSRNDALTEHQRTHTGEKPTGKK | | | |
| | | | TS | | | |
| cgggggaac | CGG ggg | 956 | LEPGEKPYKCPECGKSFSTSGEL | 1505 | X₁X₂X₃X₄X₅X₆X₇X₈X₉CX₁₀X₁₁CX₁₂X₁₃X₁₄X₁₅X₁₆TSGELVRHX₁₇X₁₈X₁₉ | 2054 |
| tggaaggga | AAC TGG | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QRAHLERHX₁₇X₁₈ | |
| gct | AAG GGA | | QRAHLERHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RKDNLKNHX₁₇ | |
| | GCT | | GKSFSRKDNLKNHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDHLTTH | |
| | | | KCPECGKSFSRSDHLTTHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DSGNLR | |
| | | | GEKPYKCPECGKSFSDSGNLRVH | | VHX₁₇X₁₈X₁₉HX₂₀X₂₁X₂₂X₂₃X₂₄X₈X₉CX₁₀X₁₁CX₁₂X₁₃X₁₄X₁₅X₁₆RSDK | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | KLVRHQRTHTGEKPYKCPECGKS | | DKLTEHX₁₇X₁₈X₁₉HX₂₀X₂₁X₂₂X₂₃X₂₄X₂₅X₂₆X₂₇X₂₈X₂₉X₃₀ | |
| | | | FSRSDKLTEHQRTHTGEKPTGKK | | | |
| | | | TS | | | |
| cccagggga | CCC AGG | 957 | LEPGEKPYKCPECGKSFSRNDAL | 1506 | X₁X₂X₃X₄X₅X₆X₇X₈X₉CX₁₀X₁₁CX₁₂X₁₃X₁₄X₁₅X₁₆RNDALTEHX₁₇X₁₈X₁₉ | 2055 |
| accgggaaa | GGA ACC | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QRANLRAHX₁₇X₁₈ | |
| ctg | ggg AAA | | QRANLRAHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDKLVRHX₁₇ | |
| | CTG | | GKSFSRSDKLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DKKDLTRH | |
| | | | KCPECGKSFSDKKDLTRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QRAHLE | |
| | | | GEKPYKCPECGKSFSQRAHLERH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDH | |
| | | | QRTHTGEKPYKCPECGKSFSRSD | | LTNHX₁₇X₁₈X₁₉HX₂₀X₂₁X₂₂X₂₃X₂₄X₈X₉CX₁₀X₁₁CX₁₂X₁₃X₁₄X₁₅X₁₆SK | |
| | | | HLTNHQRTHTGEKPYKCPECGKS | | KHLAEHX₁₇X₁₈X₁₉HX₂₀X₂₁X₂₂X₂₃X₂₄X₂₅X₂₆X₂₇X₂₈X₂₉X₃₀ | |
| | | | FSSKKHLAEHQRTHTGEKPTGKK | | | |
| | | | TS | | | |
| gctcccagg | GCT CCC | 958 | LEPGEKPYKCPECGKSFSQRANL | 1507 | X₁X₂X₃X₄X₅X₆X₇X₈X₉CX₁₀X₁₁CX₁₂X₁₃X₁₄X₁₅X₁₆QRANLRAHX₁₇X₁₈X₁₉ | 2056 |
| ggaaccggg | AGG GGA | | RAHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDKLVRHX₁₇X₁₈ | |
| aaa | ACC ggg | | RSDKLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DKKDLTRHX₁₇ | |
| | AAA | | GKSFSDKKDLTRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QRAHLERH | |
| | | | KCPECGKSFSQRAHLERHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDHLT | |
| | | | GEKPYKCPECGKSFSRSDHLTNH | | NHX₁₇X₁₈X₁₉HX₂₀X₂₁X₂₂X₂₃X₂₄X₈X₉CX₁₀X₁₁CX₁₂X₁₃X₁₄X₁₅X₁₆SKKH | |
| | | | QRTHTGEKPYKCPECGKSFSSKK | | LAEHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | HLAEHQRTHTGEKPYKCPECGKS | | GELVRHX₁₇X₁₈X₁₉HX₂₀X₂₁X₂₂X₂₃X₂₄X₂₅X₂₆X₂₇X₂₈X₂₉X₃₀ | |
| | | | FSTSGELVRHQRTHTGEKPTGKK | | | |
| | | | TS | | | |
| gggaactgg | ggg AAC | 959 | LEPGEKPYKCPECGKSFSSKKHL | 1508 | X₁X₂X₃X₄X₅X₆X₇X₈X₉CX₁₀X₁₁CX₁₂X₁₃X₁₄X₁₅X₁₆SKKHLAEHX₁₇X₁₈X₁₉ | 2057 |
| aagggagct | TGG AAG | | AEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSGELVRHX₁₇X₁₈ | |
| ccc | GGA GCT | | TSGELVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QRAHLERHX₁₇ | |
| | CCC | | GKSFSQRAHLERHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RKDNLKNH | |
| | | | KCPECGKSFSRKDNLKNHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDHLT | |
| | | | GEKPYKCPECGKSFSRSDHLTTH | | THX₁₇X₁₈X₁₉HX₂₀X₂₁X₂₂X₂₃X₂₄X₈X₉CX₁₀X₁₁CX₁₂X₁₃X₁₄X₁₅X₁₆DSGN | |
| | | | QRTHTGEKPYKCPECGKSFSDSG | | LRVHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | NLRVHQRTHTGEKPYKCPECGKS | | DKLVRHX₁₇X₁₈X₁₉HX₂₀X₂₁X₂₂X₂₃X₂₄X₂₅X₂₆X₂₇X₂₈X₂₉X₃₀ | |
| | | | FSRSDKLVRHQRTHTGEKPTGKK | | | |
| | | | TS | | | |
| aactggaag | AAC TGG | 960 | LEPGEKPYKCPECGKSFSQLAHL | 1509 | X₁X₂X₃X₄X₅X₆X₇X₈X₉CX₁₀X₁₁CX₁₂X₁₃X₁₄X₁₅X₁₆QLAHLRAHX₁₇X₁₈X₁₉ | 2058 |
| ggagctccc | AAG GGA | | RAHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆SKKHLAEHX₁₇X₁₈ | |
| aga | GCT CCC | | SKKHLAEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSGELVRHX₁₇ | |
| | AGA | | GKSFSTSGELVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QRAHLERH | |
| | | | KCPECGKSFSQRAHLERHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RKDNLK | |
| | | | GEKPYKCPECGKSFSRKDNLKNH | | NHX₁₇X₁₈X₁₉HX₂₀X₂₁X₂₂X₂₃X₂₄X₈X₉CX₁₀X₁₁CX₁₂X₁₃X₁₄X₁₅X₁₆RSDH | |
| | | | QRTHTGEKPYKCPECGKSFSRSD | | LTTHX₁₇X₁₈X₁₉HX₂₀X₂₁X₂₂X₂₃X₂₄X₈X₉CX₁₀X₁₁CX₁₂X₁₃X₁₄X₁₅X₁₆DS | |
| | | | HLTTHQRTHTGEKPYKCPECGKS | | GNLRVHX₁₇X₁₈X₁₉HX₂₀X₂₁X₂₂X₂₃X₂₄X₂₅X₂₆X₂₇X₂₈X₂₉X₃₀ | |
| | | | FSDSGNLRVHQRTHTGEKPTGKK | | | |
| | | | TS | | | |
| tggaaggga | TGG AAG | 961 | LEPGEKPYKCPECGKSFSSPADL | 1510 | X₁X₂X₃X₄X₅X₆X₇X₈X₉CX₁₀X₁₁CX₁₂X₁₃X₁₄X₁₅X₁₆SPADLTRHX₁₇X₁₈X₁₉ | 2059 |
| gctcccaga | GGA GCT | | TRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QLAHLRAHX₁₇X₁₈ | |
| aca | CCC AGA | | QLAHLRAHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆SKKHLAEHX₁₇ | |
| | ACA | | GKSFSSKKHLAEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSGELVRH | |
| | | | KCPECGKSFSTSGELVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QRAHLE | |
| | | | GEKPYKCPECGKSFSQRAHLERH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RKDN | |
| | | | QRTHTGEKPYKCPECGKSFSRKD | | LKNHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | NLKNHQRTHTGEKPYKCPECGKS | | DHLTTHX₁₇X₁₈X₁₉HX₂₀X₂₁X₂₂X₂₃X₂₄X₂₅X₂₆X₂₇X₂₈X₂₉X₃₀ | |
| | | | FSRSDHLTTHQRTHTGEKPTGKK | | | |
| | | | TS | | | |
| aagggagct | AAG GGA | 962 | LEPGEKPYKCPECGKSFSRSDHL | 1511 | X₁X₂X₃X₄X₅X₆X₇X₈X₉CX₁₀X₁₁CX₁₂X₁₃X₁₄X₁₅X₁₆RSDHLTNHX₁₇X₁₈X₁₉ | 2060 |
| cccagaaca | GCT CCC | | TNHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆SPADLTRHX₁₇X₁₈ | |
| agg | AGA ACA | | SPADLTRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QLAHLRAHX₁₇ | |
| | AGG | | GKSFSQLAHLRAHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆SKKHLAEH | |
| | | | KCPECGKSFSSKKHLAEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSGELV | |
| | | | GEKPYKCPECGKSFSTSGELVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆QRAH | |
| | | | QRTHTGEKPYKCPECGKSFSQRA | | LERHX₁₇X₁₈X₁₉HX₂₀X₂₁X₂₂X₂₃X₂₄X₈X₉CX₁₀X₁₁CX₁₂X₁₃X₁₄X₁₅X₁₆RK | |
| | | | HLERHQRTHTGEKPYKCPECGKS | | DNLKNHX₁₇X₁₈X₁₉HX₂₀X₂₁X₂₂X₂₃X₂₄X₂₅X₂₆X₂₇X₂₈X₂₉X₃₀ | |
| | | | FSRKDNLKNHQRTHTGEKPTGKK | | | |
| | | | TS | | | |
| caggggaac | CAG ggg | 963 | LEPGEKPYKCPECGKSFSRSDDL | 1512 | X₁X₂X₃X₄X₅X₆X₇X₈X₉CX₁₀X₁₁CX₁₂X₁₃X₁₄X₁₅X₁₆RSDDLVRHX₁₇X₁₈X₁₉ | 2061 |
| cgggaaact | AAC CGG | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆THLDLIRHX₁₇X₁₈ | |
| gcg | GAA ACT | | THLDLIRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QSSNLVRHX₁₇ | |
| | GCG | | GKSFSQSSNLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDKLTEH | |
| | | | KCPECGKSFSRSDKLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DSGNLR | |
| | | | GEKPYKCPECGKSFSDSGNLRVH | | VHX₁₇X₁₈X₁₉HX₂₀X₂₁X₂₂X₂₃X₂₄X₈X₉CX₁₀X₁₁CX₁₂X₁₃X₁₄X₁₅X₁₆RSDK | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RA | |
| | | | KLVRHQRTHTGEKPYKCPECGKS | | DNLTEHX₁₇X₁₈X₁₉HX₂₀X₂₁X₂₂X₂₃X₂₄X₂₅X₂₆X₂₇X₂₈X₂₉X₃₀ | |
| | | | FSRADNLTEHQRTHTGEKPTGKK | | | |
| | | | TS | | | |
| gggaaccgg | ggg AAC | 964 | LEPGEKPYKCPECGKSFSRSDKL | 1513 | X₁X₂X₃X₄X₅X₆X₇X₈X₉CX₁₀X₁₁CX₁₂X₁₃X₁₄X₁₅X₁₆RSDKLVRHX₁₇X₁₈X₁₉ | 2062 |
| gaaactgcg | CGG GAA | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDDLVRHX₁₇X₁₈ | |
| ggg | ACT GCG | | RSDDLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆THLDLTRHX₁₇ | |
| | GGG | | GKSFSTHLDLIRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QSSNLVRH | |
| | | | KCPECGKSFSQSSNLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDKLT | |
| | | | GEKPYKCPECGKSFSRSDKLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆DSGN | |
| | | | QRTHTGEKPYKCPECGKSFSDSG | | LRVHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | NLRVHQRTHTGEKPYKCPECGKS | | DKLVRHX₁₇X₁₈X₁₉HX₂₀X₂₁X₂₂X₂₃X₂₄X₂₅X₂₆X₂₇X₂₈X₂₉X₃₀ | |
| | | | FSRSDKLVRHQRTHTGEKPTGKK | | | |
| | | | TS | | | |
| aaccgggaa | AAC CGG | 965 | LEPGEKPYKCPECGKSFSQSSNL | 1514 | X₁X₂X₃X₄X₅X₆X₇X₈X₉CX₁₀X₁₁CX₁₂X₁₃X₁₄X₁₅X₁₆QSSNLVRHX₁₇X₁₈X₁₉ | 2063 |
| actgcgggg | GAA ACT | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDKLVRHX₁₇X₁₈ | |
| gaa | GCG GGG | | RSDKLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDDLVRHX₁₇ | |
| | GAA | | GKSFSRSDDLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆THLDLIRH | |
| | | | KCPECGKSFSTHLDLIRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QSSNLV | |
| | | | GEKPYKCPECGKSFSQSSNLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDK | |
| | | | QRTHTGEKPYKCPECGKSFSRSD | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆DS | |
| | | | KLTEHQRTHTGEKPYKCPECGKS | | GNLRVHX₁₇X₁₈X₁₉HX₂₀X₂₁X₂₂X₂₃X₂₄X₂₅X₂₆X₂₇X₂₈X₂₉X₃₀ | |
| | | | FSDSGNLRVHQRTHTGEKPTGKK | | | |
| | | | TS | | | |
| gcgggggaa | GCG GGG | 966 | LEPGEKPYKCPECGKSFSERSHL | 1515 | X₁X₂X₃X₄X₅X₆X₇X₈X₉CX₁₀X₁₁CX₁₂X₁₃X₁₄X₁₅X₁₆ERSHLREHX₁₇X₁₈X₁₉ | 2064 |
| ctggaaggg | GAA CTG | | REHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDKLVRHX₁₇X₁₈ | |
| agc | GAA ggg | | RSDKLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QSSNLVRHX₁₇ | |
| | AGC | | GKSFSQSSNLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RNDALTEH | |
| | | | KCPECGKSFSRNDALTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QSSNLV | |
| | | | GEKPYKCPECGKSFSQSSNLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDK | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | KLVRHQRTHTGEKPYKCPECGKS | | DDLVRHX₁₇X₁₈X₁₉HX₂₀X₂₁X₂₂X₂₃X₂₄X₂₅X₂₆X₂₇X₂₈X₂₉X₃₀ | |
| | | | FSRSDDLVRHQRTHTGEKPTGKK | | | |
| | | | TS | | | |
| actgcgggg | ACT GCG | 967 | LEPGEKPYKCPECGKSFSRSDKL | 1516 | X₁X₂X₃X₄X₅X₆X₇X₈X₉CX₁₀X₁₁CX₁₂X₁₃X₁₄X₁₅X₁₆RSDKLVRHX₁₇X₁₈X₁₉ | 2065 |
| gaactggaa | GGG GAA | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QSSNLVRHX₁₇X₁₈ | |
| ggg | CTG GAA | | QSSNLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RNDALTEHX₁₇ | |
| | GGG | | GKSFSRNDALTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QSSNLVRH | |
| | | | KCPECGKSFSQSSNLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDKLV | |
| | | | GEKPYKCPECGKSFSRSDKLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDD | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆TH | |
| | | | DLVRHQRTHTGEKPYKCPECGKS | | LDLIRHX₁₇X₁₈X₁₉HX₂₀X₂₁X₂₂X₂₃X₂₄X₂₅X₂₆X₂₇X₂₈X₂₉X₃₀ | |
| | | | FSTHLDLIRHQRTHTGEKPTGKK | | | |
| | | | TS | | | |
| cgggaaact | CGG GAA | 968 | LEPGEKPYKCPECGKSFSRNDAL | 1517 | X₁X₂X₃X₄X₅X₆X₇X₈X₉CX₁₀X₁₁CX₁₂X₁₃X₁₄X₁₅X₁₆RNDALTEHX₁₇X₁₈X₁₉ | 2066 |
| gcgggggaa | ACT GCG | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QSSNLVRHX₁₇X₁₈ | |
| ctg | GGG GAA | | QSSNLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDKLVRHX₁₇ | |
| | CTG | | GKSFSRSDKLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDDLVRH | |
| | | | KCPECGKSFSRSDDLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆THLDLI | |
| | | | GEKPYKCPECGKSFSTHLDLIRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆QSSN | |
| | | | QRTHTGEKPYKCPECGKSFSQSS | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | NLVRHQRTHTGEKPYKCPECGKS | | DKLTEHX₁₇X₁₈X₁₉HX₂₀X₂₁X₂₂X₂₃X₂₄X₂₅X₂₆X₂₇X₂₈X₂₉X₃₀ | |
| | | | FSRSDKLTEHQRTHTGEKPTGKK | | | |
| | | | TS | | | |
| gaaactgcg | GAA ACT | 969 | LEPGEKPYKCPECGKSFSQSSNL | 1518 | X₁X₂X₃X₄X₅X₆X₇X₈X₉CX₁₀X₁₁CX₁₂X₁₃X₁₄X₁₅X₁₆QSSNLVRHX₁₇X₁₈X₁₉ | 2067 |
| ggggaactg | GCG GGG | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RNDALTEHX₁₇X₁₈ | |
| gaa | GAA CTG | | RNDALTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QSSNLVRHX₁₇ | |
| | GAA | | GKSFSQSSNLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDKLVRH | |
| | | | KCPECGKSFSRSDKLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDDLV | |
| | | | GEKPYKCPECGKSFSRSDDLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆THLD | |
| | | | QRTHTGEKPYKCPECGKSFSTHL | | LIRHX₁₇X₁₈X₁₉HX₂₀X₂₁X₂₂X₂₃X₂₄X₈X₉CX₁₀X₁₁CX₁₂X₁₃X₁₄X₁₅X₁₆QS | |
| | | | DLIRHQRTHTGEKPYKCPECGKS | | SNLVRHX₁₇X₁₈X₁₉HX₂₀X₂₁X₂₂X₂₃X₂₄X₂₅X₂₆X₂₇X₂₈X₂₉X₃₀ | |
| | | | FSQSSNLVRHQRTHTGEKPTGKK | | | |
| | | | TS | | | |
| catctgggg | CAT CTG | 970 | LEPGEKPYKCPECGKSFSREDNL | 1519 | X₁X₂X₃X₄X₅X₆X₇X₈X₉CX₁₀X₁₁CX₁₂X₁₃X₁₄X₁₅X₁₆REDNLHTHX₁₇X₁₈X₁₉ | 2068 |
| cctgggatt | ggg CCT | | HTHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆HKNALQNHX₁₇X₁₈ | |
| tag | ggg ATT | | HKNALQNHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDKLVRHX₁₇ | |
| | TAG | | GKSFSRSDKLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TKNSLTEH | |
| | | | KCPECGKSFSTKNSLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDKLV | |
| | | | GEKPYKCPECGKSFSRSDKLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RNDA | |
| | | | QRTHTGEKPYKCPECGKSFSRND | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | ALTEHQRTHTGEKPYKCPECGKS | | GNLTEHX₁₇X₁₈X₁₉HX₂₀X₂₁X₂₂X₂₃X₂₄X₂₅X₂₆X₂₇X₂₈X₂₉X₃₀ | |
| | | | FSTSGNLTEHQRTHTGEKPTGKK | | | |
| | | | TS | | | |
| gattggcat | GAT TGG | 971 | LEPGEKPYKCPECGKSFSRSDKL | 1520 | X₁X₂X₃X₄X₅X₆X₇X₈X₉CX₁₀X₁₁CX₁₂X₁₃X₁₄X₁₅X₁₆RSDKLVRHX₁₇X₁₈X₁₉ | 2069 |
| ctggggcct | CAT CTG | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TKNSLTEHX₁₇X₁₈ | |
| ggg | ggg CCT | | TKNSLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDKLVRHX₁₇ | |
| | GGG | | GKSFSRSDKLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RNDALTEH | |
| | | | KCPECGKSFSRNDALTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSGNLT | |
| | | | GEKPYKCPECGKSFSTSGNLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆RSDH | |
| | | | QRTHTGEKPYKCPECGKSFSRSD | | LTTHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | HLTTHQRTHTGEKPYKCPECGKS | | GNLVRHX₁₇X₁₈X₁₉HX₂₀X₂₁X₂₂X₂₃X₂₄X₂₅X₂₆X₂₇X₂₈X₂₉X₃₀ | |
| | | | FSTSGNLVRHQRTHTGEKPTGKK | | | |
| | | | TS | | | |
| tggcatctg | TGG CAT | 972 | LEPGEKPYKCPECGKSFSHKNAL | 1521 | X₁X₂X₃X₄X₅X₆X₇X₈X₉CX₁₀X₁₁CX₁₂X₁₃X₁₄X₁₅X₁₆HKNALQNHX₁₇X₁₈X₁₉ | 2070 |
| gggcctggg | CTG ggg | | QNHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDKLVRHX₁₇X₁₈ | |
| att | CCT ggg | | RSDKLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TKNSLTEHX₁₇ | |
| | ATT | | GKSFSTKNSLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDKLVRH | |
| | | | KCPECGKSFSRSDKLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RNDALT | |
| | | | GEKPYKCPECGKSFSRNDALTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆TSGN | |
| | | | QRTHTGEKPYKCPECGKSFSTSG | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | NLTEHQRTHTGEKPYKCPECGKS | | DHLTTHX₁₇X₁₈X₁₉HX₂₀X₂₁X₂₂X₂₃X₂₄X₂₅X₂₆X₂₇X₂₈X₂₉X₃₀ | |
| | | | FSRSDHLTTHQRTHTGEKPTGKK | | | |
| | | | TS | | | |
| cagaacaag | CAG AAC | 973 | LEPGEKPYKCPECGKSFSTSGNL | 1522 | X₁X₂X₃X₄X₅X₆X₇X₈X₉CX₁₀X₁₁CX₁₂X₁₃X₁₄X₁₅X₁₆TSGNLVRHX₁₇X₁₈X₁₉ | 2071 |
| gatccagaa | AAG GAT | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QSSNLVRHX₁₇X₁₈ | |
| gat | CCA GAA | | QSSNLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSHSLTEHX₁₇ | |
| | GAT | | GKSFSTSHSLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSGNLVRH | |
| | | | KCPECGKSFSTSGNLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RKDNLK | |
| | | | GEKPYKCPECGKSFSRKDNLKNH | | NHX₁₇X₁₈X₁₉HX₂₀X₂₁X₂₂X₂₃X₂₄X₈X₉CX₁₀X₁₁CX₁₂X₁₃X₁₄X₁₅X₁₆DSGN | |
| | | | QRTHTGEKPYKCPECGKSFSDSG | | LRVHX₁₇X₁₈X₁₉HX₂₀X₂₁X₂₂X₂₃X₂₄X₈X₉CX₁₀X₁₁CX₁₂X₁₃X₁₄X₁₅X₁₆RA | |
| | | | NLRVHQRTHTGEKPYKCPECGKS | | DNLTEHX₁₇X₁₈X₁₉HX₂₀X₂₁X₂₂X₂₃X₂₄X₂₅X₂₆X₂₇X₂₈X₂₉X₃₀ | |
| | | | FSRADNLTEHQRTHTGEKPTGKK | | | |
| | | | TS | | | |
| gaagattgg | GAA GAT | 974 | LEPGEKPYKCPECGKSFSTKNSL | 1523 | X₁X₂X₃X₄X₅X₆X₇X₈X₉CX₁₀X₁₁CX₁₂X₁₃X₁₄X₁₅X₁₆TKNSLTEHX₁₇X₁₈X₁₉ | 2072 |
| catctgggg | TGG CAT | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDKLVRHX₁₇X₁₈ | |
| cct | CTG ggg | | RSDKLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RNDALTEHX₁₇ | |
| | CCT | | GKSFSRNDALTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSGNLTEH | |
| | | | KCPECGKSFSTSGNLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDHLT | |
| | | | GEKPYKCPECGKSFSRSDHLTTH | | THX₁₇X₁₈X₁₉HX₂₀X₂₁X₂₂X₂₃X₂₄X₈X₉CX₁₀X₁₁CX₁₂X₁₃X₁₄X₁₅X₁₆TSGN | |
| | | | QRTHTGEKPYKCPECGKSFSTSG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆QS | |
| | | | NLVRHQRTHTGEKPYKCPECGKS | | SNLVRHX₁₇X₁₈X₁₉HX₂₀X₂₁X₂₂X₂₃X₂₄X₂₅X₂₆X₂₇X₂₈X₂₉X₃₀ | |
| | | | FSQSSNLVRHQRTHTGEKPTGKK | | | |
| | | | TS | | | |
| aacaaggat | AAC AAG | 975 | LEPGEKPYKCPECGKSFSRSDHL | 1524 | X₁X₂X₃X₄X₅X₆X₇X₈X₉CX₁₀X₁₁CX₁₂X₁₃X₁₄X₁₅X₁₆RSDHLTTHX₁₇X₁₈X₁₉ | 2073 |
| ccagaagat | GAT CCA | | TTHQRTHTGEKPYKCPECGKSFS | | HX₂₀X₂₀X₂₁X₂₂X₂₃X₂₄X₈X₉CX₁₀X₁₁CX₁₂X₁₃X₁₄X₁₅X₁₆TSGNLVRHX₁₇X₁₈ | |
| tgg | GAA GAT | | TSGNLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QSSNLVRHX₁₇ | |
| | TGG | | GKSFSQSSNLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSHSLTEH | |
| | | | KCPECGKSFSTSHSLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSGNLV | |
| | | | GEKPYKCPECGKSFSTSGNLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RKDN | |
| | | | QRTHTGEKPYKCPECGKSFSRKD | | LKNHX₁₇X₁₈X₁₉HX₂₀X₂₁X₂₂X₂₃X₂₄X₈X₉CX₁₀X₁₁CX₁₂X₁₃X₁₄X₁₅X₁₆DS | |
| | | | NLKNHQRTHTGEKPYKCPECGKS | | GNLRVHX₁₇X₁₈X₁₉HX₂₀X₂₁X₂₂X₂₃X₂₄X₂₅X₂₆X₂₇X₂₈X₂₉X₃₀ | |
| | | | FSDSGNLRVHQRTHTGEKPTGKK | | | |
| | | | TS | | | |
| ccagaagat | CCA GAA | 976 | LEPGEKPYKCPECGKSFSRSDKL | 1525 | X₁X₂X₃X₄X₅X₆X₇X₈X₉CX₁₀X₁₁CX₁₂X₁₃X₁₄X₁₅X₁₆RSDKLVRHX₁₇X₁₈X₁₉ | 2074 |
| tggcatctg | GAT TGG | | VRHQRTHTGEKPYKCPECGKSFS | | HX₂₀X₂₀X₂₁X₂₂X₂₃X₂₄X₈X₉CX₁₀X₁₁CX₁₂X₁₃X₁₄X₁₅X₁₆RNDALTEHX₁₇X₁₈ | |
| ggg | CAT CTG | | RNDALTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSGNLTEHX₁₇ | |
| | GGG | | GKSFSTSGNLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDHLTTH | |
| | | | KCPECGKSFSRSDHLTTHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSGNLV | |
| | | | GEKPYKCPECGKSFSTSGNLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆QSSN | |
| | | | QRTHTGEKPYKCPECGKSFSQSS | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | NLVRHQRTHTGEKPYKCPECGKS | | HSLTEHX₁₇X₁₈X₁₉HX₂₀X₂₁X₂₂X₂₃X₂₄X₂₅X₂₆X₂₇X₂₈X₂₉X₃₀ | |
| | | | FSTSHSLTEHQRTHTGEKPTGKK | | | |
| | | | TS | | | |
| gatccagaa | GAT CCA | 977 | LEPGEKPYKCPECGKSFSRNDAL | 1526 | X₁X₂X₃X₄X₅X₆X₇X₈X₉CX₁₀X₁₁CX₁₂X₁₃X₁₄X₁₅X₁₆RNDALTEHX₁₇X₁₈X₁₉ | 2075 |
| gattggcat | GAA GAT | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSGNLTEHX₁₇X₁₈ | |
| ctg | TGG CAT | | TSGNLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDHLTTHX₁₇ | |
| | CTG | | GKSFSRSDHLTTHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSGNLVRH | |
| | | | KCPECGKSFSTSGNLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QSSNLV | |
| | | | GEKPYKCPECGKSFSQSSNLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆TSHS | |
| | | | QRTHTGEKPYKCPECGKSFSTSH | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | SLTEHQRTHTGEKPYKCPECGKS | | GNLVRHX₁₇X₁₈X₁₉HX₂₀X₂₁X₂₂X₂₃X₂₄X₂₅X₂₆X₂₇X₂₈X₂₉X₃₀ | |
| | | | FSTSGNLVRHQRTHTGEKPTGKK | | | |
| | | | TS | | | |
| aaggatcca | AAG GAT | 978 | LEPGEKPYKCPECGKSFSTSGNL | 1527 | X₁X₂X₃X₄X₅X₆X₇X₈X₉CX₁₀X₁₁CX₁₂X₁₃X₁₄X₁₅X₁₆TSGNLTEHX₁₇X₁₈X₁₉ | 2076 |
| gaagattgg | CCA GAA | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDHLTTHX₁₇X₁₈ | |
| cat | GAT TGG | | RSDHLTTHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSGNLVRHX₁₇ | |
| | CAT | | GKSFSTSGNLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QSSNLVRH | |
| | | | KCPECGKSFSQSSNLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSHSLT | |
| | | | GEKPYKCPECGKSFSTSHSLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆TSGN | |
| | | | QRTHTGEKPYKCPECGKSFSTSG | | LVRHX₁₇X₁₈X₁₉HX₂₀X₂₁X₂₂X₂₃X₂₄X₈X₉CX₁₀X₁₁CX₁₂XEX₁₄X₁₅X₁₆RK | |
| | | | NLVRHQRTHTGEKPYKCPECGKS | | DNLKNHX₁₇X₁₈X₁₉HX₂₀X₂₁X₂₂X₂₃X₂₄X₂₅X₂₆X₂₇X₂₈X₂₉X₃₀ | |
| | | | FSRKDNLKNHQRTHTGEKPTGKK | | | |
| | | | TS | | | |
| ctggggcct | CTG ggg | 979 | LEPGEKPYKCPECGKSFSTSGSL | 1528 | X₁X₂X₃X₄X₅X₆X₇X₈X₉CX₁₀X₁₁CX₁₂X₁₃X₁₄X₁₅X₁₆TSGSLVRHX₁₇X₁₈X₁₉ | 2077 |
| gggatttag | CCT ggg | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆REDNLHTHX₁₇X₁₈ | |
| gtt | ATT TAG | | REDNLHTHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆HKNALQNHX₁₇ | |
| | GTT | | GKSFSHKNALQNHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDKLVRH | |
| | | | KCPECGKSFSRSDKLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TKNSLT | |
| | | | GEKPYKCPECGKSFSTKNSLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆RSDK | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RN | |
| | | | KLVRHQRTHTGEKPYKCPECGKS | | DALTEHX₁₇X₁₈X₁₉HX₂₀X₂₁X₂₂X₂₃X₂₄X₂₅X₂₆X₂₇X₂₈X₂₉X₃₀ | |
| | | | FSRNDALTEHQRTHTGEKPTGKK | | | |
| | | | TS | | | |
| ctaaatcgt | CTA AAT | 980 | LEPGEKPYKCPECGKSFSDPGHL | 1529 | X₁X₂X₃X₄X₅X₆X₇X₈X₉CX₁₀X₁₁CX₁₂X₁₃X₁₄X₁₅X₁₆DPGHLVRHX₁₇X₁₈X₁₉ | 2078 |
| gggccatgg | CGT ggg | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDHLTTHX₁₇X₁₈ | |
| ggc | CCA TGG | | RSDHLTTHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSHSLTEHX₁₇ | |
| | GGC | | GKSFSTSHSLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDKLVRH | |
| | | | KCPECGKSFSRSDKLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆SRRTCR | |
| | | | GEKPYKCPECGKSFSSRRTCRAH | | AHX₁₇X₁₈X₁₉HX₂₀X₂₁X₂₂X₂₃X₂₄X₈X₉CX₁₀X₁₁CX₁₂X₁₃X₁₄X₁₅X₁₆TTGN | |
| | | | QRTHTGEKPYKCPECGKSFSTTG | | LTVHX₁₇X₁₈X₁₉HX₂₀X₂₁X₂₂X₂₃X₂₄X₈X₉CX₁₀X₁₁CX₁₂X₁₃X₁₄X₁₅X₁₆QN | |
| | | | NLTVHQRTHTGEKPYKCPECGKS | | STLTEHX₁₇X₁₈X₁₉HX₂₀X₂₁X₂₂X₂₃X₂₄X₂₅X₂₆X₂₇X₂₈X₂₉X₃₀ | |
| | | | FSQNSTLTEHQRTHTGEKPTGKK | | | |
| | | | TS | | | |
| aatcgtggg | AAT CGT | 981 | LEPGEKPYKCPECGKSFSERSHL | 1530 | X₁X₂X₃X₄X₅X₆X₇X₈X₉CX₁₀X₁₁CX₁₂X₁₃X₁₄X₁₅X₁₆ERSHLREHX₁₇X₁₈X₁₉ | 2079 |
| ccatggggc | ggg CCA | | REHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆DPGHLVRHX₁₇X₁₈ | |
| agc | TGG GGC | | DPGHLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDHLTTHX₁₇ | |
| | AGC | | GKSFSRSDHLTTHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSHSLTEH | |
| | | | KCPECGKSFSTSHSLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDKLV | |
| | | | GEKPYKCPECGKSFSRSDKLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆SRRT | |
| | | | QRTHTGEKPYKCPECGKSFSSRR | | CRAHX₁₇X₁₈X₁₉HX₂₀X₂₁X₂₂X₂₃X₂₄X₈X₉CX₁₀X₁₁CX₁₂X₁₃X₁₄X₁₅X₁₆TT | |
| | | | TCRAHQRTHTGEKPYKCPECGKS | | GNLTVHX₁₇X₁₈X₁₉HX₂₀X₂₁X₂₂X₂₃X₂₄X₂₅X₂₆X₂₇X₂₈X₂₉X₃₀ | |
| | | | FSTTGNLTVHQRTHTGEKPTGKK | | | |
| | | | TS | | | |
| cgtgggcca | CGT ggg | 982 | LEPGEKPYKCPECGKSFSTTGAL | 1531 | X₁X₂X₃X₄X₅X₆X₇X₈X₉CX₁₀X₁₁CX₁₂X₁₃X₁₄X₁₅X₁₆TTGALTEHX₁₇X₁₈X₁₉ | 2080 |
| tggggcagc | CCA TGG | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆ERSHLREHX₁₇X₁₈ | |
| ctt | GGC AGC | | ERSHLREHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DPGHLVRHX₁₇ | |
| | CTT | | GKSFSDPGHLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDHLTTH | |
| | | | KCPECGKSFSRSDHLTTHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSHSLT | |
| | | | GEKPYKCPECGKSFSTSHSLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆RSDK | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂XEX₁₄X₁₅X₁₆SR | |
| | | | KLVRHQRTHTGEKPYKCPECGKS | | RTCRAHX₁₇X₁₈X₁₉HX₂₀X₂₁X₂₂X₂₃X₂₄X₂₅X₂₆X₂₇X₂₈X₂₉X₃₀ | |
| | | | FSSRRTCRAHQRTHTGEKPTGKK | | | |
| | | | TS | | | |
| ctgcaaaag | CTG CAA | 983 | LEPGEKPYKCPECGKSFSREDNL | 1532 | X₁X₂X₃X₄X₅X₆X₇X₈X₉CX₁₀X₁₁CX₁₂X₁₃X₁₄X₁₅X₁₆REDNLHTHX₁₇X₁₈X₁₉ | 2081 |
| cattgaggg | AAG CAT | | HTHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDKLVRHX₁₇X₁₈ | |
| tag | TGA GGG | | RSDKLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QAGHLASHX₁₇ | |
| | TAG | | GKSFSQAGHLASHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSGNLTEH | |
| | | | KCPECGKSFSTSGNLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RKDNLK | |
| | | | GEKPYKCPECGKSFSRKDNLKNH | | NHX₁₇X₁₈X₁₉HX₂₀X₂₁X₂₂X₂₃X₂₄X₈X₉CX₁₀X₁₁CX₁₂X₁₃X₁₄X₁₅X₁₆QSGN | |
| | | | QRTHTGEKPYKCPECGKSFSQSG | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆RN | |
| | | | NLTEHQRTHTGEKPYKCPECGKS | | DALTEHX₁₇X₁₈X₁₉HX₂₀X₂₁X₂₂X₂₃X₂₄X₂₅X₂₆X₂₇X₂₈X₂₉X₃₀ | |
| | | | FSRNDALTEHQRTHTGEKPTGKK | | | |
| | | | TS | | | |
| caaaagcat | CAA AAG | 984 | LEPGEKPYKCPECGKSFSRKDNL | 1533 | X₁X₂X₃X₄X₅X₆X₇X₈X₉CX₁₀X₁₁CX₁₂X₁₃X₁₄X₁₅X₁₆RKDNLKNHX₁₇X₁₈X₁₉ | 2082 |
| tgagggtag | CAT TGA | | KNHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆REDNLHTHX₁₇X₁₈ | |
| aag | GGG TAG | | REDNLHTHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDKLVRHX₁₇ | |
| | APG | | GKSFSRSDKLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QAGHLASH | |
| | | | KCPECGKSFSQAGHLASHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSGNLT | |
| | | | GEKPYKCPECGKSFSTSGNLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆RKDN | |
| | | | QRTHTGEKPYKCPECGKSFSRKD | | LKNHX₁₇X₁₈X₁₉HX₂₀X₂₁X₂₂X₂₃X₂₄X₈X₉CX₁₀X₁₁CX₁₂X₁₃X₁₄X₁₅X₁₆QS | |
| | | | NLKNHQRTHTGEKPYKCPECGKS | | GNLTEHX₁₇X₁₈X₁₉HX₂₀X₂₁X₂₂X₂₃X₂₄X₂₅X₂₆X₂₇X₂₈X₂₉X₃₀ | |
| | | | FSQSGNLTEHQRTHTGEKPTGKK | | | |
| | | | TS | | | |
| aaaagcatt | AAA AGC | 985 | LEPGEKPYKCPECGKSFSHRTTL | 1534 | X₁X₂X₃X₄X₅X₆X₇X₈X₉CX₁₀X₁₁CX₁₂X₁₃X₁₄X₁₅X₁₆HRTTLTNHX₁₇X₁₈X₁₉ | 2083 |
| gagggtaga | ATT GAG | | TNHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QLAHLRAHX₁₇X₁₈ | |
| agt | GGT AGA | | QLAHLRAHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSGHLVRHX₁₇ | |
| | AGT | | GKSFSTSGHLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDNLVRH | |
| | | | KCPECGKSFSRSDNLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆HKNALQ | |
| | | | GEKPYKCPECGKSFSHKNALQNH | | NHX₁₇X₁₈X₁₉HX₂₀X₂₁X₂₂X₂₃X₂₄X₈X₉CX₁₀X₁₁CX₁₂X₁₃X₁₄X₁₅X₁₆ERSH | |
| | | | QRTHTGEKPYKCPECGKSFSERS | | LREHX₁₇X₁₈X₁₉HX₂₀X₂₁X₂₂X₂₃X₂₄X₈X₉CX₁₀X₁₁CX₁₂X₁₃X₁₄X₁₅X₁₆QR | |
| | | | HLREHQRTHTGEKPYKCPECGKS | | ANLRAHX₁₇X₁₈X₁₉HX₂₀X₂₁X₂₂X₂₃X₂₄X₂₅X₂₆X₂₇X₂₈X₂₉X₃₀ | |
| | | | FSQRANLRAHQRTHTGEKPTGKK | | | |
| | | | TS | | | |
| attgagggt | ATT GAG | 986 | LEPGEKPYKCPECGKSFSQAGHL | 1535 | X₁X₂X₃X₄X₅X₆X₇X₈X₉CX₁₀X₁₁CX₁₂X₁₃X₁₄X₁₅X₁₆QAGHLASHX₁₇X₁₈X₁₉ | 2084 |
| agaagtcaa | GGT AGA | | ASHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QSGNLTEHX₁₇X₁₈ | |
| tga | AGT CAA | | QSGNLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆HRTTLTNHX₁₇ | |
| | TGA | | GKSFSHRTTLTNHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QLAHLRAH | |
| | | | KCPECGKSFSQLAHLRAHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSGHLV | |
| | | | GEKPYKCPECGKSFSTSGHLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDN | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆HK | |
| | | | NLVRHQRTHTGEKPYKCPECGKS | | NALQNHX₁₇X₁₈X₁₉HX₂₀X₂₁X₂₂X₂₃X₂₄X₂₅X₂₆X₂₇X₂₈X₂₉X₃₀ | |
| | | | FSHKNALQNHQRTHTGEKPTGKK | | | |
| | | | TS | | | |
| agcattgag | AGC ATT | 987 | LEPGEKPYKCPECGKSFSQSGNL | 1536 | X₁X₂X₃X₄X₅X₆X₇X₈X₉CX₁₀X₁₁CX₁₂X₁₃X₁₄X₁₅X₁₆QSGNLTEHX₁₇X₁₈X₁₉ | 2085 |
| ggtagaagt | GAG GGT | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆HRTTLTNHX₁₇X₁₈ | |
| caa | AGA AGT | | HRTTLTNHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QLAHLRAHX₁₇ | |
| | CAA | | GKSFSQLAHLRAHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSGHLVRH | |
| | | | KCPECGKSFSTSGHLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDNLV | |
| | | | GEKPYKCPECGKSFSRSDNLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆HKNA | |
| | | | QRTHTGEKPYKCPECGKSFSHKN | | LQNHX₁₇X₁₈X₁₉HX₂₀X₂₁X₂₂X₂₃X₂₄X₈X₉CX₁₀X₁₁CX₁₂X₁₃X₁₄X₁₅X₁₆ER | |
| | | | ALQNHQRTHTGEKPYKCPECGKS | | SHLREHX₁₇X₁₈X₁₉HX₂₀X₂₁X₂₂X₂₃X₂₄X₂₅X₂₆X₂₇X₂₈X₂₉X₃₀ | |
| | | | FSERSHLREHQRTHTGEKPTGKK | | | |
| | | | TS | | | |
| atgatttgg | ATG ATT | 988 | LEPGEKPYKCPECGKSFSQSSNL | 1537 | X₁X₂X₃X₄X₅X₆X₇X₈X₉CX₁₀X₁₁CX₁₂X₁₃X₁₄X₁₅X₁₆QSSNLVRHX₁₇X₁₈X₁₉ | 2086 |
| gaagttatt | TGG GAA | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆HKNALQNHX₁₇X₁₈ | |
| gaa | GTT ATT | | HKNALQNHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSGSLVRHX₁₇ | |
| | GAA | | GKSFSTSGSLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QSSNLVRH | |
| | | | KCPECGKSFSQSSNLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDHLT | |
| | | | GEKPYKCPECGKSFSRSDHLTTH | | THX₁₇X₁₈X₁₉HX₂₀X₂₁X₂₂X₂₃X₂₄X₈X₉CX₁₀X₁₁CX₁₂X₁₃X₁₄X₁₅X₁₆HKNA | |
| | | | QRTHTGEKPYKCPECGKSFSHKN | | LQNHX₁₇X₁₈X₁₉HX₂₀X₂₁X₂₂X₂₃X₂₄X₈X₉CX₁₀X₁₁CX₁₂X₁₃X₁₄X₁₅X₁₆RR | |
| | | | ALQNHQRTHTGEKPYKCPECGKS | | DELNVHX₁₇X₁₈X₁₉HX₂₀X₂₁X₂₂X₂₃X₂₄X₂₅X₂₆X₂₇X₂₈X₂₉X₃₀ | |
| | | | FSRRDELNVHQRTHTGEKPTGKK | | | |
| | | | TS | | | |
| taggctgtc | TAG GCT | 989 | LEPGEKPYKCPECGKSFSREDNL | 1538 | X₁X₂X₃X₄X₅X₆X₇X₈X₉CX₁₀X₁₁CX₁₂X₁₃X₁₄X₁₅X₁₆REDNLHTHX₁₇X₁₈X₁₉ | 2087 |
| agtgcctga | GTC AGT | | HTHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QAGHLASHX₁₇X₁₈ | |
| tag | GCC TGA | | QAGHLASHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DCRDLARHX₁₇ | |
| | TAG | | GKSFSDCRDLARHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆HRTTLTNH | |
| | | | KCPECGKSFSHRTTLTNHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DPGALV | |
| | | | GEKPYKCPECGKSFSDPGALVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆TSGE | |
| | | | QRTHTGEKPYKCPECGKSFSTSG | | LVRHX₁₇X₁₈X₁₉HX₂₀X₂₁X₂₂X₂₃X₂₄X₈X₉CX₁₀X₁₁CX₁₂XEX₁₄X₁₅X₁₆RE | |
| | | | ELVRHQRTHTGEKPYKCPECGKS | | DNLHTHX₁₇X₁₈X₁₉HX₂₀X₂₁X₂₂X₂₃X₂₄X₂₅X₂₆X₂₇X₂₈X₂₉X₃₀ | |
| | | | FSREDNLHTHQRTHTGEKPTGKK | | | |
| | | | TS | | | |
| aggtaggct | AGG TAG | 990 | LEPGEKPYKCPECGKSFSQAGHL | 1539 | X₁X₂X₃X₄X₅X₆X₇X₈X₉CX₁₀X₁₁CX₁₂X₁₃X₁₄X₁₅X₁₆QAGHLASHX₁₇X₁₈X₁₉ | 2088 |
| gtcagtgcc | GCT GTC | | ASHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆DCRDLARHX₁₇X₁₈ | |
| tga | AGT GCC | | DCRDLARHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆HRTTLTNHX₁₇ | |
| | TGA | | GKSFSHRTTLTNHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DPGALVRH | |
| | | | KCPECGKSFSDPGALVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSGELV | |
| | | | GEKPYKCPECGKSFSTSGELVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆REDN | |
| | | | QRTHTGEKPYKCPECGKSFSRED | | LHTHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | NLHTHQRTHTGEKPYKCPECGKS | | DHLTNHX₁₇X₁₈X₁₉HX₂₀X₂₁X₂₂X₂₃X₂₄X₂₅X₂₆X₂₇X₂₈X₂₉X₃₀ | |
| | | | FSRSDHLTNHQRTHTGEKPTGKK | | | |
| | | | TS | | | |
| cggaggtag | CGG AGG | 991 | LEPGEKPYKCPECGKSFSDCRDL | 1540 | X₁X₂X₃X₄X₅X₆X₇X₈X₉CX₁₀X₁₁CX₁₂X₁₃X₁₄X₁₅X₁₆DCRDLARHX₁₇X₁₈X₁₉ | 2089 |
| gctgtcagt | TAG GCT | | ARHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆HRTTLTNHX₁₇X₁₈ | |
| gcc | GTC AGT | | HRTTLTNHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DPGALVRHX₁₇ | |
| | GCC | | GKSFSDPGALVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSGELVRH | |
| | | | KCPECGKSFSTSGELVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆REDNLH | |
| | | | GEKPYKCPECGKSFSREDNLHTH | | THX₁₇X₁₈X₁₉HX₂₀X₂₁X₂₂X₂₃X₂₄X₈X₉CX₁₀X₁₁CX₁₂X₁₃X₁₄X₁₅X₁₆RSDH | |
| | | | QRTHTGEKPYKCPECGKSFSRSD | | LTNHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | HLTNHQRTHTGEKPYKCPECGKS | | DKLTEHX₁₇X₁₈X₁₉HX₂₀X₂₁X₂₂X₂₃X₂₄X₂₅X₂₆X₂₇X₂₈X₂₉X₃₀ | |
| | | | FSRSDKLTEHQRTHTGEKPTGKK | | | |
| | | | TS | | | |
| tagaatgcc | TAG AAT | 992 | LEPGEKPYKCPECGKSFSRSDEL | 1541 | X₁X₂X₃X₄X₅X₆X₇X₈X₉CX₁₀X₁₁CX₁₂X₁₃X₁₄X₁₅X₁₆RSDELVRHX₁₇X₁₈X₁₉ | 2090 |
| tgacttggg | GCC TGA | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDKLVRHX₁₇X₁₈ | |
| gtg | CTT GGG | | RSDKLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TTGALTEHX₁₇ | |
| | GTG | | GKSFSTTGALTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QAGHLASH | |
| | | | KCPECGKSFSQAGHLASHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DCRDLA | |
| | | | GEKPYKCPECGKSFSDCRDLARH | | RHX₁₇X₁₈X₁₉HX₂₀X₂₁X₂₂X₂₃X₂₄X₈X₉CX₁₀X₁₁CX₁₂X₁₃X₁₄X₁₅X₁₆TTGN | |
| | | | QRTHTGEKPYKCPECGKSFSTTG | | LTVHX₁₇X₁₈X₁₉HX₂₀X₂₁X₂₂X₂₃X₂₄X₈X₉CX₁₀X₁₁CX₁₂X₁₃X₁₄X₁₅X₁₆RE | |
| | | | NLTVHQRTHTGEKPYKCPECGKS | | DNLHTHX₁₇X₁₈X₁₉HX₂₀X₂₁X₂₂X₂₃X₂₄X₂₅X₂₆X₂₇X₂₈X₂₉X₃₀ | |
| | | | FSREDNLHTHQRTHTGEKPTGKK | | | |
| | | | TS | | | |
| aatgcctga | AAT GCC | 993 | LEPGEKPYKCPECGKSFSSPADL | 1542 | X₁X₂X₃X₄X₅X₆X₇X₈X₉CX₁₀X₁₁CX₁₂X₁₃X₁₄X₁₅X₁₆SPADLTRHX₁₇X₁₈X₁₉ | 2091 |
| cttggggtg | TGA CTT | | TRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDELVRHX₁₇X₁₈ | |
| aca | GGG gtg | | RSDELVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDKLVRHX₁₇ | |
| | ACA | | GKSFSRSDKLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TTGALTEH | |
| | | | KCPECGKSFSTTGALTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QAGHLA | |
| | | | GEKPYKCPECGKSFSQAGHLASH | | SHX₁₇X₁₈X₁₉HX₂₀X₂₁X₂₂X₂₃X₂₄X₈X₉CX₁₀X₁₁CX₁₂X₁₃X₁₄X₁₅X₁₆DCRD | |
| | | | QRTHTGEKPYKCPECGKSFSDCR | | LARHX₁₇X₁₈X₁₉HX₂₀X₂₁X₂₂X₂₃X₂₄X₈X₉CX₁₀X₁₁CX₁₂XEX₁₄X₁₅X₁₆TT | |
| | | | DLARHQRTHTGEKPYKCPECGKS | | GNLTVHX₁₇X₁₈X₁₉HX₂₀X₂₁X₂₂X₂₃X₂₄X₂₅X₂₆X₂₇X₂₈X₂₉X₃₀ | |
| | | | FSTTGNLTVHQRTHTGEKPTGKK | | | |
| | | | TS | | | |
| agttagaat | AGT TAG | 994 | LEPGEKPYKCPECGKSFSRSDKL | 1543 | X₁X₂X₃X₄X₅X₆X₇X₈X₉CX₁₀X₁₁CX₁₂X₁₃X₁₄X₁₅X₁₆RSDKLVRHX₁₇X₁₈X₁₉ | 2092 |
| gcctgactt | AT GCC | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TTGALTEHX₁₇X₁₈ | |
| ggg | TGA CTT | | TTGALTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QAGHLASHX₁₇ | |
| | GGG | | GKSFSQAGHLASHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DCRDLARH | |
| | | | KCPECGKSFSDCRDLARHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TTGNLT | |
| | | | GEKPYKCPECGKSFSTTGNLTVH | | VHX₁₇X₁₈X₁₉HX₂₀X₂₁X₂₂X₂₃X₂₄X₈X₉CX₁₀X₁₁CX₁₂X₁₃X₁₄X₁₅X₁₆REDN | |
| | | | QRTHTGEKPYKCPECGKSFSRED | | LHTHX₁₇X₁₈X₁₉HX₂₀X₂₁X₂₂X₂₃X₂₄X₈X₉CX₁₀X₁₁CX₁₂X₁₃X₁₄X₁₅X₁₆HR | |
| | | | NLHTHQRTHTGEKPYKCPECGKS | | TTLTNHX₁₇X₁₈X₁₉HX₂₀X₂₁X₂₂X₂₃X₂₄X₂₅X₂₆X₂₇X₂₈X₂₉X₃₀ | |
| | | | FSHRTTLTNHQRTHTGEKPTGKK | | | |
| | | | TS | | | |
| gcctgactt | GCC TGA | 995 | LEPGEKPYKCPECGKSFSRRDEL | 1544 | X₁X₂X₃X₄X₅X₆X₇X₈X₉CX₁₀X₁₁CX₁₂X₁₃X₁₄X₁₅X₁₆RRDELNVHX₁₇X₁₈X₁₉ | 2093 |
| ggggtgaca | CTT GGG | | NVHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆SPADLTRHX₁₇X₁₈ | |
| atg | gtg ACA | | SPADLTRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDELVRHX₁₇ | |
| | ATG | | GKSFSRSDELVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDKLVRH | |
| | | | KCPECGKSFSRSDKLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TTGALT | |
| | | | GEKPYKCPECGKSFSTTGALTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆QAGH | |
| | | | QRTHTGEKPYKCPECGKSFSQAG | | LASHX₁₇X₁₈X₁₉HX₂₀X₂₁X₂₂X₂₃X₂₄X₈X₉CX₁₀X₁₁CX₁₂X₁₃X₁₄X₁₅X₁₆DC | |
| | | | HLASHQRTHTGEKPYKCPECGKS | | RDLARHX₁₇X₁₈X₁₉HX₂₀X₂₁X₂₂X₂₃X₂₄X₂₅X₂₆X₂₇X₂₈X₂₉X₃₀ | |
| | | | FSDCRDLARHQRTHTGEKPTGKK | | | |
| | | | TS | | | |
| ggggtgaca | GGG gtg | 996 | LEPGEKPYKCPECGKSFSRSDHL | 1545 | X₁X₂X₃X₄X₅X₆X₇X₈X₉CX₁₀X₁₁CX₁₂X₁₃X₁₄X₁₅X₁₆RSDHLTNHX₁₇X₁₈X₁₉ | 2094 |
| atggcttgg | ACA ATG | | TNHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDHLTTHX₁₇X₁₈ | |
| agg | GCT TGG | | RSDHLTTHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSGELVRHX₁₇ | |
| | AGG | | GKSFSTSGELVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RRDELNVH | |
| | | | KCPECGKSFSRRDELNVHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆SPADLT | |
| | | | GEKPYKCPECGKSFSSPADLTRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDE | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | ELVRHQRTHTGEKPYKCPECGKS | | DKLVRHX₁₇X₁₈X₁₉HX₂₀X₂₁X₂₂X₂₃X₂₄X₂₅X₂₆X₂₇X₂₈X₂₉X₃₀ | |
| | | | FSRSDKLVRHQRTHTGEKPTGKK | | | |
| | | | TS | | | |
| tgacttggg | TGA CTT | 997 | LEPGEKPYKCPECGKSFSTSGEL | 1546 | X₁X₂X₃X₄X₅X₆X₇X₈X₉CX₁₀X₁₁CX₁₂X₁₃X₁₄X₁₅X₁₆TSGELVRHX₁₇X₁₈X₁₉ | 2095 |
| gtgacaatg | GGG gtg | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RRDELNVHX₁₇X₁₈ | |
| gct | ACA ATG | | RRDELNVHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆SPADLTRHX₁₇ | |
| | GCT | | GKSFSSPADLTRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDELVRH | |
| | | | KCPECGKSFSRSDELVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDKLV | |
| | | | GEKPYKCPECGKSFSRSDKLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆TTGA | |
| | | | QRTHTGEKPYKCPECGKSFSTTG | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆QA | |
| | | | ALTEHQRTHTGEKPYKCPECGKS | | GHLASHX₁₇X₁₈X₁₉HX₂₀X₂₁X₂₂X₂₃X₂₄X₂₅X₂₆X₂₇X₂₈X₂₉X₃₀ | |
| | | | FSQAGHLASHQRTHTGEKPTGKK | | | |
| | | | TS | | | |
| cttggggtg | CTT GGG | 130 | LEPGEKPYKCPECGKSFSRSDHL | 131 | X₁X₂X₃X₄X₅X₆X₇X₈X₉CX₁₀X₁₁CX₁₂X₁₃X₁₄X₁₅X₁₆RSDHLTTHX₁₇X₁₈X₁₉ | 320 |
| acaatggct | gtg ACA | | TTHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSGELVRHX₁₇X₁₈ | |
| tgg | ATG GCT | | TSGELVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RRDELNVHX₁₇ | |
| | TGG | | GKSFSRRDELNVHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆SPADLTRH | |
| | | | KCPECGKSFSSPADLTRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDELV | |
| | | | GEKPYKCPECGKSFSRSDELVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDK | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂XEX₁₄X₁₅X₁₆TT | |
| | | | KLVRHQRTHTGEKPYKCPECGKS | | GALTEHX₁₇X₁₈X₁₉HX₂₀X₂₁X₂₂X₂₃X₂₄X₂₅X₂₆X₂₇X₂₈X₂₉X₃₀ | |
| | | | FSTTGALTEHQRTHTGEKPTGKK | | | |
| | | | TS | | | |
| gcttggagg | GCT TGG | 998 | LEPGEKPYKCPECGKSFSDPGAL | 1547 | X₁X₂X₃X₄X₅X₆X₇X₈X₉CX₁₀X₁₁CX₁₂X₁₃X₁₄X₁₅X₁₆DPGALVRHX₁₇X₁₈X₁₉ | 2096 |
| ggtgggtga | AGG GGT | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QAGHLASHX₁₇X₁₈ | |
| gtc | GGG TGA | | QAGHLASHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDKLVRHX₁₇ | |
| | GTC | | GKSFSRSDKLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSGHLVRH | |
| | | | KCPECGKSFSTSGHLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDHLT | |
| | | | GEKPYKCPECGKSFSRSDHLTNH | | NHX₁₇X₁₈X₁₉HX₂₀X₂₁X₂₂X₂₃X₂₄X₈X₉CX₁₀X₁₁CX₁₂X₁₃X₁₄X₁₅X₁₆RSDH | |
| | | | QRTHTGEKPYKCPECGKSFSRSD | | LTTHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | HLTTHQRTHTGEKPYKCPECGKS | | GELVRHX₁₇X₁₈X₁₉HX₂₀X₂₁X₂₂X₂₃X₂₄X₂₅X₂₆X₂₇X₂₈X₂₉X₃₀ | |
| | | | FSTSGELVRHQRTHTGEKPTGKK | | | |
| | | | TS | | | |
| tggaggggt | TGG AGG | 999 | LEPGEKPYKCPECGKSFSRKDNL | 1548 | X₁X₂X₃X₄X₅X₆X₇X₈X₉CX₁₀X₁₁CX₁₂X₁₃X₁₄X₁₅X₁₆RKDNLKNHX₁₇X₁₈X₁₉ | 2097 |
| gggtgagtc | GGT GGG | | KNHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆DPGALVRHX₁₇X₁₈ | |
| aag | TGA GTC | | DPGALVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QAGHLASHX₁₇ | |
| | APG | | GKSFSQAGHLASHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDKLVRH | |
| | | | KCPECGKSFSRSDKLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSGHLV | |
| | | | GEKPYKCPECGKSFSTSGHLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDH | |
| | | | QRTHTGEKPYKCPECGKSFSRSD | | LTNHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | HLTNHQRTHTGEKPYKCPECGKS | | DHLTTHX₁₇X₁₈X₁₉HX₂₀X₂₁X₂₂X₂₃X₂₄X₂₅X₂₆X₂₇X₂₈X₂₉X₃₀ | |
| | | | FSRSDHLTTHQRTHTGEKPTGKK | | | |
| | | | TS | | | |
| gtgacaatg | gtg ACA | 1000 | LEPGEKPYKCPECGKSFSTSGHL | 1549 | X₁X₂X₃X₄X₅X₆X₇X₈X₉CX₁₀X₁₁CX₁₂X₁₃X₁₄X₁₅X₁₆TSGHLVRHX₁₇X₁₈X₁₉ | 2098 |
| gcttggagg | ATG GCT | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDHLTNHX₁₇X₁₈ | |
| ggt | TGG AGG | | RSDHLTNHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDHLTTHX₁₇ | |
| | GGT | | GKSFSRSDHLTTHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSGELVRH | |
| | | | KCPECGKSFSTSGELVRHQRTHT | | X₂₇X₁₈X₁₉HX₂₀X₂₁X₂₂X₂₃X₂₄X₈X₉CX₁₀X₁₁CX₁₂X₁₃X₁₄X₁₅X₁₆RRDELN | |
| | | | GEKPYKCPECGKSFSRRDELNVH | | VHX₁₇X₁₈X₁₉HX₂₀X₂₁X₂₂X₂₃X₂₄X₈X₉CX₁₀X₁₁CX₁₂X₁₃X₁₄X₁₅X₁₆SPAD | |
| | | | QRTHTGEKPYKCPECGKSFSSPA | | LTRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | DLTRHQRTHTGEKPYKCPECGKS | | DELVRHX₁₇X₁₈X₁₉HX₂₀X₂₁X₂₂X₂₃X₂₄X₂₅X₂₆X₂₇X₂₈X₂₉X₃₀ | |
| | | | FSRSDELVRHQRTHTGEKPTGKK | | | |
| | | | TS | | | |
| atggcttgg | ATG GCT | 1001 | LEPGEKPYKCPECGKSFSQAGHL | 1550 | X₁X₂X₃X₄X₅X₆X₇X₈X₉CX₁₀X₁₁CX₁₂X₁₃X₁₄X₁₅X₁₆QAGHLASHX₁₇X₁₈X₁₉ | 2099 |
| aggggtggg | TGG AGG | | ASHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDKLVRHX₁₇X₁₈ | |
| tga | GGT GGG | | RSDKLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSGHLVRHX₁₇ | |
| | TGA | | GKSFSTSGHLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDHLTNH | |
| | | | KCPECGKSFSRSDHLTNHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDHLT | |
| | | | GEKPYKCPECGKSFSRSDHLTTH | | THX₁₇X₁₈X₁₉HX₂₀X₂₁X₂₂X₂₃X₂₄X₈X₉CX₁₀X₁₁CX₁₂X₁₃X₁₄X₁₅X₁₆TSGE | |
| | | | QRTHTGEKPYKCPECGKSFSTSG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RR | |
| | | | ELVRHQRTHTGEKPYKCPECGKS | | DELNVHX₁₇X₁₈X₁₉HX₂₀X₂₁X₂₂X₂₃X₂₄X₂₅X₂₆X₂₇X₂₈X₂₉X₃₀ | |
| | | | FSRRDELNVHQRTHTGEKPTGKK | | | |
| | | | TS | | | |
| acaatggct | ACA ATG | 1002 | LEPGEKPYKCPECGKSFSRSDKL | 1551 | X₁X₂X₃X₄X₅X₆X₇X₈X₉CX₁₀X₁₁CX₁₂X₁₃X₁₄X₁₅X₁₆RSDKLVRHX₁₇X₁₈X₁₉ | 2100 |
| tggaggggt | GCT TGG | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSGHLVRHX₁₇X₁₈ | |
| ggg | AGG GGT | | TSGHLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDHLTNHX₁₇ | |
| | GGG | | GKSFSRSDHLTNHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDHLTTH | |
| | | | KCPECGKSFSRSDHLTTHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSGELV | |
| | | | GEKPYKCPECGKSFSTSGELVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RRDE | |
| | | | QRTHTGEKPYKCPECGKSFSRRD | | LNVHX₁₇X₁₈X₁₉HX₂₀X₂₁X₂₂X₂₃X₂₄X₈X₉CX₁₀X₁₁CX₁₂X₁₃X₁₄X₁₅X₁₆SP | |
| | | | ELNVHQRTHTGEKPYKCPECGKS | | ADLTRHX₁₇X₁₈X₁₉HX₂₀X₂₁X₂₂X₂₃X₂₄X₂₅X₂₆X₂₇X₂₈X₂₉X₃₀ | |
| | | | FSSPADLTRHQRTHTGEKPTGKK | | | |
| | | | TS | | | |
| gggtgagtc | GGG TGA | 1003 | LEPGEKPYKCPECGKSFSRRDEL | 1552 | X₁X₂X₃X₄X₅X₆X₇X₈X₉CX₁₀X₁₁CX₁₂X₁₃X₁₄X₁₅X₁₆RRDELNVHX₁₇X₁₈X₁₉ | 2101 |
| aagggtcaa | GTC AAG | | NVHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QSGNLTEHX₁₇X₁₈ | |
| atg | GGT CAA | | QSGNLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSGHLVRHX₁₇ | |
| | ATG | | GKSFSTSGHLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RKDNLKNH | |
| | | | KCPECGKSFSRKDNLKNHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DPGALV | |
| | | | GEKPYKCPECGKSFSDPGALVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆QAGH | |
| | | | QRTHTGEKPYKCPECGKSFSQAG | | LASHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | HLASHQRTHTGEKPYKCPECGKS | | DKLVRHX₁₇X₁₈X₁₉HX₂₀X₂₁X₂₂X₂₃X₂₄X₂₅X₂₆X₂₇X₂₈X₂₉X₃₀ | |
| | | | FSRSDKLVRHQRTHTGEKPTGKK | | | |
| | | | TS | | | |
| ggtcaaatg | GGT CAA | 1004 | LEPGEKPYKCPECGKSFSRSDNL | 1553 | X₁X₂X₃X₄X₅X₆X₇X₈X₉CX₁₀X₁₁CX₁₂X₁₃X₁₄X₁₅X₁₆RSDNLVRHX₁₇X₁₈X₁₉ | 2102 |
| agtgcccgt | ATG AGT | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆SRRTCRAHX₁₇X₁₈ | |
| gag | GCC CGT | | SRRTCRAHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DCRDLARHX₁₇ | |
| | GAG | | GKSFSDCRDLARHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆HRTTLTNH | |
| | | | KCPECGKSFSHRTTLTNHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RRDELN | |
| | | | GEKPYKCPECGKSFSRRDELNVH | | VHX₁₇X₁₈X₁₉HX₂₀X₂₁X₂₂X₂₃X₂₄X₈X₉CX₁₀X₁₁CX₁₂X₁₃X₁₄X₁₅X₁₆QSGN | |
| | | | QRTHTGEKPYKCPECGKSFSQSG | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | NLTEHQRTHTGEKPYKCPECGKS | | GHLVRHX₁₇X₁₈X₁₉HX₂₀X₂₁X₂₂X₂₃X₂₄X₂₅X₂₆X₂₇X₂₈X₂₉X₃₀ | |
| | | | FSTSGHLVRHQRTHTGEKPTGKK | | | |
| | | | TS | | | |
| tgagtcaag | TGA GTC | 1005 | LEPGEKPYKCPECGKSFSHRTTL | 1554 | X₁X₂X₃X₄X₅X₆X₇X₈X₉CX₁₀X₁₁CX₁₂X₁₃X₁₄X₁₅X₁₆HRTTLTNHX₁₇X₁₈X₁₉ | 2103 |
| ggtcaaatg | AAG GGT | | TNHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RRDELNVHX₁₇X₁₈ | |
| agt | CAA ATG | | RRDELNVHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QSGNLTEHX₁₇ | |
| | AGT | | GKSFSQSGNLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSGHLVRH | |
| | | | KCPECGKSFSTSGHLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RKDNLK | |
| | | | GEKPYKCPECGKSFSRKDNLKNH | | NHX₁₇X₁₈X₁₉HX₂₀X₂₁X₂₂X₂₃X₂₄X₈X₉CX₁₀X₁₁CX₁₂X₁₃X₁₄X₁₅X₁₆DPGA | |
| | | | QRTHTGEKPYKCPECGKSFSDPG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆QA | |
| | | | ALVRHQRTHTGEKPYKCPECGKS | | GHLASHX₁₇X₁₈X₁₉HX₂₀X₂₁X₂₂X₂₃X₂₄X₂₅X₂₆X₂₇X₂₈X₂₉X₃₀ | |
| | | | FSQAGHLASHQRTHTGEKPTGKK | | | |
| | | | TS | | | |
| gtcaagggt | GTC AAG | 1006 | LEPGEKPYKCPECGKSFSDCRDL | 1555 | X₁X₂X₃X₄X₅X₆X₇X₈X₉CX₁₀X₁₁CX₁₂X₁₃X₁₄X₁₅X₁₆DCRDLARHX₁₇X₁₈X₁₉ | 2104 |
| caaatgagt | GGT CAA | | ARHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆HRTTLTNHX₁₇X₁₈ | |
| gcc | ATG AGT | | HRTTLTNHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RRDELNVHX₁₇ | |
| | GCC | | GKSFSRRDELNVHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QSGNLTEH | |
| | | | KCPECGKSFSQSGNLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSGHLV | |
| | | | GEKPYKCPECGKSFSTSGHLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RKDN | |
| | | | QRTHTGEKPYKCPECGKSFSRKD | | LKNHX₁₇X₁₈X₁₉HX₂₀X₂₁X₂₂X₂₃X₂₄X₈X₉CX₁₀X₁₁CX₁₂X₁₃X₁₄X₁₅X₁₆DP | |
| | | | NLKNHQRTHTGEKPYKCPECGKS | | GALVRHX₁₇X₁₈X₁₉HX₂₀X₂₁X₂₂X₂₃X₂₄X₂₅X₂₆X₂₇X₂₈X₂₉X₃₀ | |
| | | | FSDPGALVRHQRTHTGEKPTGKK | | | |
| | | | TS | | | |
| aagggtcaa | AAG GGT | 1007 | LEPGEKPYKCPECGKSFSSRRTC | 1556 | X₁X₂X₃X₄X₅X₆X₇X₈X₉CX₁₀X₁₁CX₁₂X₁₃X₁₄X₁₅X₁₆SRRTCRAHX₁₇X₁₈X₁₉ | 2105 |
| atgagtgcc | CAA ATG | | RAHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆DCRDLARHX₁₇X₁₈ | |
| cgt | AGT GCC | | DCRDLARHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆HRTTLTNHX₁₇ | |
| | CGT | | GKSFSHRTTLTNHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RRDELNVH | |
| | | | KCPECGKSFSRRDELNVHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QSGNLT | |
| | | | GEKPYKCPECGKSFSQSGNLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆TSGH | |
| | | | QRTHTGEKPYKCPECGKSFSTSG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RK | |
| | | | HLVRHQRTHTGEKPYKCPECGKS | | DNLKNHX₁₇X₁₈X₁₉HX₂₀X₂₁X₂₂X₂₃X₂₄X₂₅X₂₆X₂₇X₂₈X₂₉X₃₀ | |
| | | | FSRKDNLKNHQRTHTGEKPTGKK | | | |
| | | | TS | | | |
| aggggtggg | AGG GGT | 1008 | LEPGEKPYKCPECGKSFSTSGHL | 1557 | X₁X₂X₃X₄X₅X₆X₇X₈X₉CX₁₀X₁₁CX₁₂X₁₃X₁₄X₁₅X₁₆TSGHLVRHX₁₇X₁₈X₁₉ | 2106 |
| tgagtcaag | GGG TGA | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RKDNLKNHX₁₇X₁₈ | |
| ggt | GTC AAG | | RKDNLKNHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DPGALVRHX₁₇ | |
| | GGT | | GKSFSDPGALVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QAGHLASH | |
| | | | KCPECGKSFSQAGHLASHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDKLV | |
| | | | GEKPYKCPECGKSFSRSDKLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆TSGH | |
| | | | QRTHTGEKPYKCPECGKSFSTSG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | HLVRHQRTHTGEKPYKCPECGKS | | DHLTNHX₁₇X₁₃X₁₉HX₂₀X₂₁X₂₂X₂₃X₂₄X₂₅X₂₆X₂₇X₂₈X₂₉X₃₀ | |
| | | | FSRSDHLTNHQRTHTGEKPTGKK | | | |
| | | | TS | | | |
| ggtgggtga | GGT GGG | 1009 | LEPGEKPYKCPECGKSFSQSGNL | 1558 | X₁X₂X₃X₄X₅X₆X₇X₈X₉CX₁₀X₁₁CX₁₂X₁₃X₁₄X₁₅X₁₆QSGNLTEHX₁₇X₁₈X₁₉ | 2107 |
| gtcaagggt | TGA GTC | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSGHLVRHX₁₇X₁₈ | |
| caa | AAG GGT | | TSGHLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RKDNLKNHX₁₇ | |
| | CAA | | GKSFSRKDNLKNHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DPGALVRH | |
| | | | KCPECGKSFSDPGALVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QAGHLA | |
| | | | GEKPYKCPECGKSFSQAGHLASH | | SHX₁₇X₁₈X₁₉HX₂₀X₂₁X₂₂X₂₃X₂₄X₈X₉CX₁₀X₁₁CX₁₂X₁₃X₁₄X₁₅X₁₆RSDK | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | KLVRHQRTHTGEKPYKCPECGKS | | GHLVRHX₁₇X₁₈X₁₉HX₂₀X₂₁X₂₂X₂₃X₂₄X₂₅X₂₆X₂₇X₂₈X₂₉X₃₀ | |
| | | | FSTSGHLVRHQRTHTGEKPTGKK | | | |
| | | | TS | | | |
| ctgacttgg | CTG ACT | 1010 | LEPGEKPYKCPECGKSFSDPGHL | 1559 | X₁X₂X₃X₄X₅X₆X₇X₈X₉CX₁₀X₁₁CX₁₂X₁₃X₁₄X₁₅X₁₆DPGHLVRHX₁₇X₁₈X₁₉ | 2108 |
| ggtgacaat | TGG GGT | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TTGNLTVHX₁₇X₁₈ | |
| ggc | GAC AAT | | TTGNLTVHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DPGNLVRHX₁₇ | |
| | GGC | | GKSFSDPGNLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSGHLVRH | |
| | | | KCPECGKSFSTSGHLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDHLT | |
| | | | GEKPYKCPECGKSFSRSDHLTTH | | THX₁₇X₁₈X₁₉HX₂₀X₂₁X₂₂X₂₃X₂₄X₈X₉CX₁₀X₁₁CX₁₂X₁₃X₁₄X₁₅X₁₆THLD | |
| | | | QRTHTGEKPYKCPECGKSFSTHL | | LIRHX₁₇X₁₈X₁₉HX₂₀X₂₁X₂₂X₂₃X₂₄X₈X₉CX₁₀X₁₁CX₁₂X₁₃X₁₄X₁₅X₁₆RN | |
| | | | DLIRHQRTHTGEKPYKCPECGKS | | DALTEHX₁₇X₁₈X₁₉XHX₂₀X₂₁X₂₂X₂₃X₂₄X₂₅X₂₆X₂₇X₂₈X₂₉X₃₀ | |
| | | | FSRNDALTEHQRTHTGEKPTGKK | | | |
| | | | TS | | | |
| gggtgacaa | GGG TGA | 1011 | LEPGEKPYKCPECGKSFSRSDKL | 1560 | X₁X₂X₃X₄X₅X₆X₇X₈X₉CXₗ₀X₁₁CX₁₂X₁₃X₁₄X₁₅X₁₆RSDKLVRHX₁₇X₁₈X₁₉ | 2109 |
| tggcttgga | CAA TGG | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QRAHLERHX₁₇X₁₈ | |
| ggg | CTT GGA | | QRAHLERHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TTGALTEHX₁₇ | |
| | GGG | | GKSFSTTGALTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDHLTTH | |
| | | | KCPECGKSFSRSDHLTTHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QSGNLT | |
| | | | GEKPYKCPECGKSFSQSGNLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆QAGH | |
| | | | QRTHTGEKPYKCPECGKSFSQAG | | LASHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | HLASHQRTHTGEKPYKCPECGKS | | DKLVRHX₁₇X₁₈X₁₉HX₂₀X₂₁X₂₂X₂₃X₂₄X₂₅X₂₆X₂₇X₂₈X₂₉X₃₀ | |
| | | | FSRSDKLVRHQRTHTGEKPTGKK | | | |
| | | | TS | | | |
| tgacaatgg | TGA CAA | 1012 | LEPGEKPYKCPECGKSFSRSDEL | 1561 | X₁X₂X₃X₄X₅X₆X₇X₈X₉CX₁₀X₁₁CX₁₂X₁₃X₁₄X₁₅X₁₆RSDELVRHX₁₇X₁₈X₁₉ | 2110 |
| cttggaggg | TGG CTT | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDKLVRHX₁₇X₁₈ | |
| gtg | GGA GGG | | RSDKLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QRAHLERHX₁₇ | |
| | GTG | | GKSFSQRAHLERHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TTGALTEH | |
| | | | KCPECGKSFSTTGALTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDHLT | |
| | | | GEKPYKCPECGKSFSRSDHLTTH | | THX₁₇X₁₈X₁₉HX₂₀X₂₁X₂₂X₂₃X₂₄X₈X₉CX₁₀X₁₁CX₁₂X₁₃X₁₄X₁₅X₁₆QSGN | |
| | | | QRTHTGEKPYKCPECGKSFSQSG | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆QA | |
| | | | NLTEHQRTHTGEKPYKCPECGKS | | GHLASHX₁₇X₁₈X₁₉HX₂₀X₂₁X₂₂X₂₃X₂₄X₂₅X₂₆X₂₇X₂₈X₂₉X₃₀ | |
| | | | FSQAGHLASHQRTHTGEKPTGKK | | | |
| | | | TS | | | |
| tggcttgga | TGG CTT | 1013 | LEPGEKPYKCPECGKSFSRSDNL | 1562 | X₁X₂X₃X₄X₅X₆X₇X₈X₉CX₁₀X₁₁CX₁₂X₁₃X₁₄X₁₅X₁₆RSDNLVRHX₁₇X₁₈X₁₉ | 2111 |
| ggggtgggt | GGA GGG | | VRHQRTHTGEKPYKCPECGKSFS | | HX₂₀X₂₁X₂₂X₂₃X₂₄X₈X₉CX₁₀X₁₁CX₁₂X₁₃X₁₄Z15X₁₆TSGHLVRHX₁₇X₁₈ | |
| gag | GTG GGT | | TSGHLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDELVRHX₁₇ | |
| | GAG | | GKSFSRSDELVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDKLVRH | |
| | | | KCPECGKSFSRSDKLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QRAHLE | |
| | | | GEKPYKCPECGKSFSQRAHLERH | | RHX₁₇X₁₈X₁₉HX₂₀X₂₁X₂₂X₂₃X₂₄X₈X₉CX₁₀X₁₁CX₁₂X₁₃X₁₄X₁₅X₁₆TTGA | |
| | | | QRTHTGEKPYKCPECGKSFSTTG | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | ALTEHQRTHTGEKPYKCPECGKS | | DHLTTHX₁₇X₁₈X₁₉HX₂₀X₂₁X₂₂X₂₃X₂₄X₂₅X₂₆X₂₇X₂₈X₂₉X₃₀ | |
| | | | FSRSDHLTTHQRTHTGEKPTGKK | | | |
| | | | TS | | | |
| caatggctt | CAA TGG | 1014 | LEPGEKPYKCPECGKSFSTSGHL | 1563 | X₁X₂X₃X₄X₅X₆X₇X₈X₉CX₁₀X₁₁CX₁₂X₁₃X₁₄X₁₅X₁₆TSGHLVRHX₁₇X₁₈X₁₉ | 2112 |
| ggaggggtg | CTT GGA | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDELVRHX₁₇X₁₈ | |
| ggt | GGG GTG | | RSDELVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDKLVRHX₁₇ | |
| | GGT | | GKSFSRSDKLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QRAHLERH | |
| | | | KCPECGKSFSQRAHLERHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TTGALT | |
| | | | GEKPYKCPECGKSFSTTGALTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆RSDH | |
| | | | QRTHTGEKPYKCPECGKSFSRSD | | LTTHX₁₇X₁₈X₁₉HX₂₀X₂₁X₂₂X₂₃X₂₄X₈X₉CX₁₀X₁₁CX₁₂X₁₃X₁₄X₁₅X₁₆QS | |
| | | | HLTTHQRTHTGEKPYKCPECGKS | | GNLTEHX₁₇X₁₈X₁₉HX₂₀X₂₁X₂₂X₂₃X₂₄X₂₅X₂₆X₂₇X₂₈X₂₉X₃₀ | |
| | | | FSQSGNLTEHQRTHTGEKPTGKK | | | |
| | | | TS | | | |
| gaggggtgg | GAG GGG | 1015 | LEPGEKPYKCPECGKSFSRSDKL | 1564 | X₁X₂X₃X₄X₅X₆X₇X₈X₉CX₁₀X₁₁CX₁₂X₁₃X₁₄X₁₅X₁₆RSDKLVRHX₁₇X₁₈X₁₉ | 2113 |
| gtgagtcaa | TGG gtg | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QSGNLTEHX₁₇X₁₈ | |
| ggg | AGT CAA | | QSGNLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆HRTTLTNHX₁₇ | |
| | GGG | | GKSFSHRTTLTNHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDELVRH | |
| | | | KCPECGKSFSRSDELVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDHLT | |
| | | | GEKPYKCPECGKSFSRSDHLTTH | | THX₁₇X₁₈X₁₉HX₂₀X₂₁X₂₂X₂₃X₂₄X₈X₉CX₁₀X₁₁CX₁₂X₁₃X₁₄X₁₅X₁₆RSDK | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | KLVRHQRTHTGEKPYKCPECGKS | | DNLVRHX₁₇X₁₈X₁₉HX₂₀X₂₁X₂₂X₂₃X₂₄X₂₅X₂₆X₂₇X₂₈X₂₉X₃₀ | |
| | | | FSRSDNLVRHQRTHTGEKPTGKK | | | |
| | | | TS | | | |
| agggtcaaa | AGG GTC | 1016 | LEPGEKPYKCPECGKSFSRSDEL | 1565 | X₁X₂X₃X₄X₅X₆X₇X₈X₉CX₁₀X₁₁CX₁₂X₁₃X₁₄X₁₅X₁₆RSDELVRHX₁₇X₁₈X₁₉ | 2114 |
| tgagtgccc | AAA TGA | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆SKKHLAEHX₁₇X₁₈ | |
| gtg | gtg CCC | | SKKHLAEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDELVRHX₁₇ | |
| | GTG | | GKSFSRSDELVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QAGHLASH | |
| | | | KCPECGKSFSQAGHLASHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QRANLR | |
| | | | GEKPYKCPECGKSFSQRANLRAH | | AHX₁₇X₁₈X₁₉HX₂₀X₂₁X₂₂X₂₃X₂₄X₈X₉CX₁₀X₁₁CX₁₂X₁₃X₁₄X₁₅X₁₆DPGA | |
| | | | QRTHTGEKPYKCPECGKSFSDPG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | ALVRHQRTHTGEKPYKCPECGKS | | DHLTNHX₁₇X₁₈X₁₉HX₂₀X₂₁X₂₂X₂₃X₂₄X₂₅X₂₆X₂₇X₂₈X₂₉X₃₀ | |
| | | | FSRSDHLTNHQRTHTGEKPTGKK | | | |
| | | | TS | | | |
| gtcaaatga | GTC AAA | 1017 | LEPGEKPYKCPECGKSFSHRTTL | 1566 | X₁X₂X₃X₄X₅X₆X₇X₈X₉CX₁₀X₁₁CX₁₂X₁₃X₁₄X₁₅X₁₆HRTTLTNHX₁₇X₁₈X₁₉ | 2115 |
| gtgcccgtg | TGA gtg | | TNHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDELVRHX₁₇X₁₈ | |
| agt | CCC gtg | | RSDELVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆SKKHLAEHX₁₇ | |
| | AGT | | GKSFSSKKHLAEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDELVRH | |
| | | | KCPECGKSFSRSDELVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QAGHLA | |
| | | | GEKPYKCPECGKSFSQAGHLASH | | SHX₁₇X₁₈X₁₉HX₂₀X₂₁X₂₂X₂₃X₂₄X₈X₉CX₁₀X₁₁CX₁₂X₁₃X₁₄X₁₅X₁₆QRAN | |
| | | | QRTHTGEKPYKCPECGKSFSQRA | | LRAHX₁₇X₁₈X₁₉HX₂₀X₂₁X₂₂X₂₃X₂₄X₈X₉CX₁₀X₁₁CX₁₂X₁₃X₁₄X₁₅X₁₆DP | |
| | | | NLRAHQRTHTGEKPYKCPECGKS | | GALVRHX₁₇X₁₈X₁₉HX₂₀X₂₁X₂₂X₂₃X₂₄X₂₅X₂₆X₂₇X₂₈X₂₉X₃₀ | |
| | | | FSDPGALVRHQRTHTGEKPTGKK | | | |
| | | | TS | | | |
| aaatgagtg | AAA TGA | 1018 | LEPGEKPYKCPECGKSFSTSGNL | 1567 | X₁X₂X₃X₄X₅X₆X₇X₈X₉CX₁₀X₁₁CX₁₂X₁₃X₁₄X₁₅X₁₆TSGNLTEHX₁₇X₁₈X₁₉ | 2116 |
| cccgtgagt | gtg CCC | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆HRTTLTNHX₁₇X₁₈ | |
| cat | gtg AGT | | HRTTLTNHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDELVRHX₁₇ | |
| | CAT | | GKSFSRSDELVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆SKKHLAEH | |
| | | | KCPECGKSFSSKKHLAEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDELV | |
| | | | GEKPYKCPECGKSFSRSDELVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆QAGH | |
| | | | QRTHTGEKPYKCPECGKSFSQAG | | LASHX₁₇X₁₈X₁₉HX₂₀X₂₁X₂₂X₂₃X₂₄X₈X₉CX₁₀X₁₁CX₁₂X₁₃X₁₄X₁₅X₁₆QR | |
| | | | HLASHQRTHTGEKPYKCPECGKS | | ANLRAHX₁₇X₁₈X₁₉HX₂₀X₂₁X₂₂X₂₃X₂₄X₂₅X₂₆X₂₇X₂₈X₂₉X₃₀ | |
| | | | FSQRANLRAHQRTHTGEKPTGKK | | | |
| | | | TS | | | |
| tgagtgccc | TGA gtg | 1019 | LEPGEKPYKCPECGKSFSTSGNL | 1568 | X₁X₂X₃X₄X₅X₆X₇X₈X₉CX₁₀X₁₁CX₁₂X₁₃X₁₄X₁₅X₁₆TSGNLVRHX₁₇X₁₈X₁₉ | 2117 |
| gtgagtcat | CCC gtg | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSGNLTEHX₁₇X₁₈ | |
| gat | AGT CAT | | TSGNLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆HRTTLTNHX₁₇ | |
| | GAT | | GKSFSHRTTLTNHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDELVRH | |
| | | | KCPECGKSFSRSDELVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆SKKHLA | |
| | | | GEKPYKCPECGKSFSSKKHLAEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆RSDE | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆QA | |
| | | | ELVRHQRTHTGEKPYKCPECGKS | | GHLASHX₁₇X₁₈X₁₉HX₂₀X₂₁X₂₂X₂₃X₂₄X₂₅X₂₆X₂₇X₂₈X₂₉X₃₀ | |
| | | | FSQAGHLASHQRTHTGEKPTGKK | | | |
| | | | TS | | | |
| gtgcccgtg | gtg CCC | 1020 | LEPGEKPYKCPECGKSFSDCRDL | 1569 | X₁X₂X₃X₄X₅X₆X₇X₈X₉CX₁₀X₁₁CX₁₂X₁₃X₁₄X₁₅X₁₆DCRDLARHX₁₇X₁₈X₁₉ | 2118 |
| agtcatgat | gtg AGT | | ARHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSGNLVRHX₁₇X₁₈ | |
| gcc | CAT GAT | | TSGNLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSGNLTEHX₁₇ | |
| | GCC | | GKSFSTSGNLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆HRTTLTNH | |
| | | | KCPECGKSFSHRTTLTNHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDELV | |
| | | | GEKPYKCPECGKSFSRSDELVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆SKKH | |
| | | | QRTHTGEKPYKCPECGKSFSSKK | | LAEHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | HLAEHQRTHTGEKPYKCPECGKS | | DELVRHX₁₇X₁₈X₁₉HX₂₀X₂₁X₂₂X₂₃X₂₄X₂₅X₂₆X₂₇X₂₈X₂₉X₃₀ | |
| | | | FSRSDELVRHQRTHTGEKPTGKK | | | |
| | | | TS | | | |
| ccgtgagtc | CCG TGA | 1021 | LEPGEKPYKCPECGKSFSDCRDL | 1570 | X₁X₂X₃X₄X₅X₆X₇X₈X₉CX₁₀X₁₁CX₁₂X₁₃X₁₄X₁₅X₁₆DCRDLARHX₁₇X₁₈X₁₉ | 2119 |
| atgatgcct | GTC ATG | | ARHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TKNSLTEHX₁₇X₁₈ | |
| gcc | ATG CCT | | TKNSLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RRDELNVHX₁₇ | |
| | GCC | | GKSFSRRDELNVHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RRDELNVH | |
| | | | KCPECGKSFSRRDELNVHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DPGALV | |
| | | | GEKPYKCPECGKSFSDPGALVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆QAGH | |
| | | | QRTHTGEKPYKCPECGKSFSQAG | | LASHX₁₇X₁₈X₁₉HX₂₀X₂₁X₂₂X₂₃X₂₄X₈X₉CX₁₀X₁₁CX₁₂X₁₃X₁₄X₁₅X₁₆RN | |
| | | | HLASHQRTHTGEKPYKCPECGKS | | DTLTEHX₁₇X₁₈X₁₉HX₂₀X₂₁X₂₂X₂₃X₂₄X₂₅X₂₆X₂₇X₂₈X₂₉X₃₀ | |
| | | | FSRNDTLTEHQRTHTGEKPTGKK | | | |
| | | | TS | | | |
| gtcagccag | GTC AGC | 1022 | LEPGEKPYKCPECGKSFSDSGNL | 1571 | X₁X₂X₃X₄X₅X₆X₇X₈X₉CX₁₀X₁₁CX₁₂X₁₃X₁₄X₁₅X₁₆DSGNLRVHX₁₇X₁₈X₁₉ | 2120 |
| aaaccaaca | CAG AAA | | RVHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆SPADLTRHX₁₇X₁₈ | |
| aac | CCA ACA | | SPADLTRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSHSLTEHX₁₇ | |
| | AAC | | GKSFSTSHSLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QRANLRAH | |
| | | | KCPECGKSFSQRANLRAHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RADNLT | |
| | | | GEKPYKCPECGKSFSRADNLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆ERSH | |
| | | | QRTHTGEKPYKCPECGKSFSERS | | LREHX₁₇X₁₈X₁₉HX₂₀X₂₁X₂₂X₂₃X₂₄X₈X₉CX₁₀X₁₁CX₁₂X₁₃X₁₄X₁₅X₁₆DP | |
| | | | HLREHQRTHTGEKPYKCPECGKS | | GALVRHX₁₇X₁₈X₁₉HX₂₀X₂₁X₂₂X₂₃X₂₄X₂₅X₂₆X₂₇X₂₈X₂₉X₃₀ | |
| | | | FSDPGALVRHQRTHTGEKPTGKK | | | |
| | | | TS | | | |
| agccagaaa | AGC CAG | 1023 | LEPGEKPYKCPECGKSFSERSHL | 1572 | X₁X₂X₃X₄X₅X₆X₇X₈X₉CX₁₀X₁₁CX₁₂X₁₃X₁₄X₁₅X₁₆ERSHLREHX₁₇X₁₈X₁₉ | 2121 |
| ccaacaaac | AAA CCA | | REHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆DSGNLRVHX₁₇X₁₈ | |
| agc | ACA AAC | | DSGNLRVHQRTHTGEKPYKCPEC | | X9HX₂₀X₂₁X₂₂X₂₃X₂₄X₈X₉CX₁₀X₁₁CX₁₂X₁₃X₁₄X₁₅X₁₆SPADLTRHX₁₇ | |
| | AGC | | GKSFSSPADLTRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSHSLTEH | |
| | | | KCPECGKSFSTSHSLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QRANLR | |
| | | | GEKPYKCPECGKSFSQRANLRAH | | AHX₁₇X₁₈X₁₉HX₂₀X₂₁X₂₂X₂₃X₂₄X₈X₉CX₁₀X₁₁CX₁₂X₁₃X₁₄X₁₅X₁₆RADN | |
| | | | QRTHTGEKPYKCPECGKSFSRAD | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆ER | |
| | | | NLTEHQRTHTGEKPYKCPECGKS | | SHLREHX₁₇X₁₈X₁₉HX₂₀X₂₁X₂₂X₂₃X₂₄X₂₅X₂₆X₂₇X₂₈X₂₉X₃₀ | |
| | | | FSERSHLREHQRTHTGEKPTGKK | | | |
| | | | TS | | | |
| cagaaacca | CAG AAA | 1024 | LEPGEKPYKCPECGKSFSQSGNL | 1573 | X₁X₂X₃X₄X₅X₆X₇X₈X₉CX₁₀X₁₁CX₁₂X₁₃X₁₄X₁₅X₁₆QSGNLTEHX₁₇X₁₈X₁₉ | 2122 |
| acaaacagc | CCA ACA | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆ERSHLREHX₁₇X₁₈ | |
| caa | AAC AGC | | ERSHLREHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DSGNLRVHX₁₇ | |
| | CAA | | GKSFSDSGNLRVHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆SPADLTRH | |
| | | | KCPECGKSFSSPADLTRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSHSLT | |
| | | | GEKPYKCPECGKSFSTSHSLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆QRAN | |
| | | | QRTHTGEKPYKCPECGKSFSQRA | | LRAHX₁₇X₁₈X₁₉HX₂₀X₂₁X₂₂X₂₃X₂₄X₈X₉CX₁₀X₁₁CX₁₂X₁₃X₁₄X₁₅X₁₆RA | |
| | | | NLRAHQRTHTGEKPYKCPECGKS | | DNLTEHX₁₇X₁₈X₁₉HX₂₀X₂₁X₂₂X₂₃X₂₄X₂₅X₂₆X₂₇X₂₈X₂₉X₃₀ | |
| | | | FSRADNLTEHQRTHTGEKPTGKK | | | |
| | | | TS | | | |
| gccccagca | GCC CCA | 1025 | LEPGEKPYKCPECGKSFSERSHL | 1574 | X₁X₂X₃X₄X₅X₆X₇X₈X₉CX₁₀X₁₁CX₁₂X₁₃X₁₄X₁₅X₁₆ERSHLREHX₁₇X₁₈X₁₉ | 2123 |
| gttgtaatt | GCA GTT | | REHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆HKNALQNHX₁₇X₁₈ | |
| agc | GTA ATT | | HKNALQNHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QSSSLVRHX₁₇ | |
| | AGC | | GKSFSQSSSLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSGSLVRH | |
| | | | KCPECGKSFSTSGSLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QSGDLR | |
| | | | GEKPYKCPECGKSFSQSGDLRRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆TSHS | |
| | | | QRTHTGEKPYKCPECGKSFSTSH | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆DC | |
| | | | SLTEHQRTHTGEKPYKCPECGKS | | RDLARHX₁₇X₁₈X₁₉HX₂₀X₂₁X₂₂X₂₃X₂₄X₂₅X₂₆X₂₇X₂₈X₂₉X3 | |
| | | | FSDCRDLARHQRTHTGEKPTGKK | | | |
| | | | TS | | | |
| cggtgagtt | CGG TGA | 1026 | LEPGEKPYKCPECGKSFSQSGDL | 1575 | X₁X₂X₃X₄X₅X₆X₇X₈X₉CX₁₀X₁₁CX₁₂X₁₃X₁₄X₁₅X₁₆QSGDLRRHX₁₇X₁₈X₁₉ | 2124 |
| agggcccca | GTT AGG | | RRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSHSLTEHX₁₇X₁₈ | |
| gca | GCC CCA | | TSHSLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DCRDLARHX₁₇ | |
| | GCA | | GKSFSDCRDLARHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDHLTNH | |
| | | | KCPECGKSFSRSDHLTNHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSGSLV | |
| | | | GEKPYKCPECGKSFSTSGSLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆QAGH | |
| | | | QRTHTGEKPYKCPECGKSFSQAG | | LASHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | HLASHQRTHTGEKPYKCPECGKS | | DKLTEHX₁₇X₁₈X₁₉HX₂₀X₂₁X₂₂X₂₃X₂₄X₂₅X₂₆X₂₇X₂₈X₂₉X₃₀ | |
| | | | FSRSDKLTEHQRTHTGEKPTGKK | | | |
| | | | TS | | | |
| ggtgtcagc | GGT GTC | 1027 | LEPGEKPYKCPECGKSFSSPADL | 1576 | X₁X₂X₃X₄X₅X₆X₇X₈X₉CX₁₀X₁₁CX₁₂X₁₃X₁₄X₁₅X₁₆SPADLTRHX₁₇X₁₈X₁₉ | 2125 |
| cagaaacca | AGC CAG | | TRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSHSLTEHX₁₇X₁₈ | |
| aca | AAA CCA | | TSHSLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QRANLRAHX₁₇ | |
| | ACA | | GKSFSQRANLRAHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RADNLTEH | |
| | | | KCPECGKSFSRADNLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆ERSHLR | |
| | | | GEKPYKCPECGKSFSERSHLREH | | EHX₁₇X₁₈X₁₉HX₂₀X₂₁X₂₂X₂₃X₂₄X₈X₉CX₁₀X₁₁CX₁₂X₁₃X₁₄X₁₅X₁₆DPGA | |
| | | | QRTHTGEKPYKCPECGKSFSDPG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | ALVRHQRTHTGEKPYKCPECGKS | | GHLVRHX₁₇X₁₈X₁₉HX₂₀X₂₁X₂₂X₂₃X₂₄X₂₅X₂₆X₂₇X₂₈X₂₉X₃₀ | |
| | | | FSTSGHLVRHQRTHTGEKPTGKK | | | |
| | | | TS | | | |
| agggcccca | AGG GCC | 1028 | LEPGEKPYKCPECGKSFSHKNAL | 1577 | X₁X₂X₃X₄X₅X₆X₇X₈X₉CX₁₀X₁₁CX₁₂X₁₃X₁₄X₁₅X₁₆HKNALQNHX₁₇X₁₈X₁₉ | 2126 |
| gcagttgta | CCA GCA | | QNHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QSSSLVRHX₁₇X₁₈ | |
| att | GTT GTA | | QSSSLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSGSLVRHX₁₇ | |
| | ATT | | GKSFSTSGSLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QSGDLRRH | |
| | | | KCPECGKSFSQSGDLRRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSHSLT | |
| | | | GEKPYKCPECGKSFSTSHSLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆DCRD | |
| | | | QRTHTGEKPYKCPECGKSFSDCR | | LARHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | DLARHQRTHTGEKPYKCPECGKS | | DHLTNHX₁₇X₁₈X₁₉HX₂₀X₂₁X₂₂X₂₃X₂₄X₂₅X₂₆X₂₇X₂₈X₂₉X₃₀ | |
| | | | FSRSDHLTNHQRTHTGEKPTGKK | | | |
| | | | TS | | | |
| ccagcagtt | CCA GCA | 1029 | LEPGEKPYKCPECGKSFSDKKDL | 1578 | X₁X₂X₃X₄X₅X₆X₇X₈X₉CX₁₀X₁₁CX₁₂X₁₃X₁₄X₁₅X₁₆DKKDLTRHX₁₇X₁₈X₁₉ | 2127 |
| gtaattagc | GTT GTA | | TRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆ERSHLREHX₁₇X₁₈ | |
| acc | ATT AGC | | ERSHLREHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆HKNALQNHX₁₇ | |
| | ACC | | GKSFSHKNALQNHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QSSSLVRH | |
| | | | KCPECGKSFSQSSSLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSGSLV | |
| | | | GEKPYKCPECGKSFSTSGSLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆QSGD | |
| | | | QRTHTGEKPYKCPECGKSFSQSG | | LRRHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | DLRRHQRTHTGEKPYKCPECGKS | | HSLTEHX₁₇X₁₈X₁₉HX₂₀X₂₁X₂₂X₂₃X₂₄X₂₅X₂₆X₂₇X₂₈X₂₉X₃₀ | |
| | | | FSTSHSLTEHQRTHTGEKPTGKK | | | |
| | | | TS | | | |
| ataactgaa | ATA ACT | 1030 | LEPGEKPYKCPECGKSFSTSGSL | 1579 | X₁X₂X₃X₄X₅X₆X₇X₈X₉CX₁₀X₁₁CX₁₂X₁₃X₁₄X₁₅X₁₆TSGSLVRHX₁₇X₁₈X₁₉ | 2128 |
| catcggtga | GAA CAT | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QAGHLASHX₁₇X₁₈ | |
| gtt | CGG TGA | | QAGHLASHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDKLTEHX₁₇ | |
| | GTT | | GKSFSRSDKLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSGNLTEH | |
| | | | KCPECGKSFSTSGNLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QSSNLV | |
| | | | GEKPYKCPECGKSFSQSSNLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆THLD | |
| | | | QRTHTGEKPYKCPECGKSFSTHL | | LIRHX₁₇X₁₈X₁₉HX₂₀X₂₁X₂₂X₂₃X₂₄X₈X₉CX₁₀X₁₁CX₁₂X₁₃X₁₄X₁₅X₁₆QK | |
| | | | DLIRHQRTHTGEKPYKCPECGKS | | SSLIAHX₁₇X₁₈X₁₉HX₂₀X₂₁X₂₂X₂₃X₂₄X₂₅X₂₆X₂₇X₂₈X₂₉X₃₀ | |
| | | | FSQKSSLIAHQRTHTGEKPTGKK | | | |
| | | | TS | | | |
| catcggtga | CAT CGG | 1031 | LEPGEKPYKCPECGKSFSTSHSL | 1580 | X₁X₂X₃X₄X₅X₆X₇X₈X₉CX₁₀X₁₁CX₁₂X₁₃X₁₄X₁₅X₁₆TSHSLTEHX₁₇X₁₈X₁₉ | 2129 |
| gttagggcc | TGA GTT | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆DCRDLARHX₁₇X₁₈ | |
| cca | AGG GCC | | DCRDLARHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDHLTNHX₁₇ | |
| | CCA | | GKSFSRSDHLTNHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSGSLVRH | |
| | | | KCPECGKSFSTSGSLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QAGHLA | |
| | | | GEKPYKCPECGKSFSQAGHLASH | | SHX₁₇X₁₈X₁₉HX₂₀X₂₁X₂₂X₂₃X₂₄X₈X₉CX₁₀X₁₁CX₁₂X₁₃X₁₄X₁₅X₁₆RSDK | |
| | | | QRTHTGEKPYKCPECGKSFSRSD | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | KLTEHQRTHTGEKPYKCPECGKS | | GNLTEHX₁₇X₁₈X₁₉HX₂₀X₂₁X₂₂X₂₃X₂₄X₂₅X₂₆X₂₇X₂₈X₂₉X₃₀ | |
| | | | FSTSGNLTEHQRTHTGEKPTGKK | | | |
| | | | TS | | | |
| ccgggtgtc | CCG GGT | 122 | LEPGEKPYKCPECGKSFSTSHSL | 123 | X₁X₂X₃X₄X₅X₆X₇X₈X₉CX₁₀X₁₁CX₁₂X₁₃X₁₄X₁₅X₁₆TSHSLTEHX₁₇X₁₈X₁₉ | 318 |
| agccagaaa | GTC AGC | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QRANLRAHX₁₇X₁₈ | |
| cca | CAG AAA | | QRANLRAHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RADNLTEHX₁₇ | |
| | CCA | | GKSFSRADNLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆ERSHLREH | |
| | | | KCPECGKSFSERSHLREHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DPGALV | |
| | | | GEKPYKCPECGKSFSDPGALVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆TSGH | |
| | | | QRTHTGEKPYKCPECGKSFSTSG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RN | |
| | | | HLVRHQRTHTGEKPYKCPECGKS | | DTLTEHX₁₇X₁₈X₁₉HX₂₀X₂₁X₂₂X₂₃X₂₄X₂₅X₂₆X₂₇X₂₈X₂₉X₃₀ | |
| | | | FSRNDTLTEHQRTHTGEKPTGKK | | | |
| | | | TS | | | |
| actgaacat | ACT GAA | 126 | LEPGEKPYKCPECGKSFSRSDHL | 127 | X₁X₂X₃X₄X₅X₆X₇X₈X₉CX₁₀X₁₁CX₁₂X₁₃X₁₄X₁₅X₁₆RSDHLTNHX₁₇X₁₈X₁₉ | 319 |
| cggtgagtt | CAT CGG | | TNHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSGSLVRHX₁₇X₁₈ | |
| agg | TGA GTT | | TSGSLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QAGHLASHX₁₇ | |
| | AGG | | GKSFSQAGHLASHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDKLTEH | |
| | | | KCPECGKSFSRSDKLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSGNLT | |
| | | | GEKPYKCPECGKSFSTSGNLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆QSSN | |
| | | | QRTHTGEKPYKCPECGKSFSQSS | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆TH | |
| | | | NLVRHQRTHTGEKPYKCPECGKS | | LDLIRHX₁₇X₁₈X₁₉HX₂₀X₂₁X₂₂X₂₃X₂₄X₂₅X₂₆X₂₇X₂₈X₂₉X₃₀ | |
| | | | FSTHLDLIRHQRTHTGEKPTGKK | | | |
| | | | TS | | | |
| gaacatcgg | GAA CAT | 1032 | LEPGEKPYKCPECGKSFSDCRDL | 1581 | X₁X₂X₃X₄X₅X₆X₇X₈X₉CX₁₀X₁₁CX₁₂X₁₃X₁₄X₁₅X₁₆DCRDLARHX₁₇X₁₈X₁₉ | 2130 |
| tgagttagg | CGG TGA | | ARHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDHLTNHX₁₇X₁₈ | |
| gcc | GTT AGG | | RSDHLTNHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSGSLVRHX₁₇ | |
| | GCC | | GKSFSTSGSLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QAGHLASH | |
| | | | KCPECGKSFSQAGHLASHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDKLT | |
| | | | GEKPYKCPECGKSFSRSDKLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆TSGN | |
| | | | QRTHTGEKPYKCPECGKSFSTSG | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆QS | |
| | | | NLTEHQRTHTGEKPYKCPECGKS | | SNLVRHX₁₇X₁₈X₁₉HX₂₀X₂₁X₂₂X₂₃X₂₄X₂₅X₂₆X₂₇X₂₈X₂₉X₃₀ | |
| | | | FSQSSNLVRHQRTHTGEKPTGKK | | | |
| | | | TS | | | |
| accccgggt | ACC CCG | 1033 | LEPGEKPYKCPECGKSFSQRANL | 1582 | X₁X₂X₃X₄X₅X₆X₇X₈X₉CX₁₀X₁₁CX₁₂X₁₃X₁₄X₁₅X₁₆QRANLRAHX₁₇X₁₈X₁₉ | 2131 |
| gtcagccag | GGT GTC | | RAHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RADNLTEHX₁₇X₁₈ | |
| aaa | AGC CAG | | RADNLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆ERSHLREHX₁₇ | |
| | AAA | | GKSFSERSHLREHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DPGALVRH | |
| | | | KCPECGKSFSDPGALVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSGHLV | |
| | | | GEKPYKCPECGKSFSTSGHLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RNDT | |
| | | | QRTHTGEKPYKCPECGKSFSRND | | LTEHX₁XX9HX₂₀X₂₁X₂₂X₂₃X₂₄X₈X₉CX₁₀X₁₁CX₁₂X₁₃X₁₄X₁₅X₁₆DK | |
| | | | TLTEHQRTHTGEKPYKCPECGKS | | KDLTRHX₁₇X₁₈X₁₉HX₂₀X₂₁X₂₂X₂₃X₂₄X₂₅X₂₆X₂₇X₂₈X₂₉X₃₀ | |
| | | | FSDKKDLTRHQRTHTGEKPTGKK | | | |
| | | | TS | | | |
| agcaccccg | AGC ACC | 1034 | LEPGEKPYKCPECGKSFSRADNL | 1583 | X₁X₂X₃X₄X₅X₆X₇X₈X₉CX₁₀X₁₁CX₁₂X₁₃X₁₄X₁₅X₁₆RADNLTEHX₁₇X₁₈X₁₉ | 2132 |
| ggtgtcagc | CCG GGT | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆ERSHLREHX₁₇X₁₈ | |
| cag | GTC AGC | | ERSHLREHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DPGALVRHX₁₇ | |
| | CAG | | GKSFSDPGALVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSGHLVRH | |
| | | | KCPECGKSFSTSGHLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RNDTLT | |
| | | | GEKPYKCPECGKSFSRNDTLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆DKKD | |
| | | | QRTHTGEKPYKCPECGKSFSDKK | | LTRHX₁₇X₁₈X₁₉HX₂₀X₂₁X₂₂X₂₃X₂₄X₈X₉CX₁₀X₁₁CX₁₂X₁₃X₁₄X₁₅X₁₆ER | |
| | | | DLTRHQRTHTGEKPYKCPECGKS | | SHLREHX₁₇X₁₈X₁₉HX₂₀X₂₁X₂₂X₂₃X₂₄X₂₅X₂₆X₂₇X₂₈X₂₉X₃₀ | |
| | | | FSERSHLREHQRTHTGEKPTGKK | | | |
| | | | TS | | | |
| gcagttgta | GCA GTT | 1035 | LEPGEKPYKCPECGKSFSRNDTL | 1584 | X₁X₂X₃X₄X₅X₆X₇X₈X₉CX₁₀X₁₁CX₁₂X₁₃X₁₄X₁₅X₁₆RNDTLTEHX₁₇X₁₈X₁₉ | 2133 |
| attagcacc | GTA ATT | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆DKKDLTRHX₁₇X₁₈ | |
| ccg | AGC ACC | | DKKDLTRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆ERSHLREHX₁₇ | |
| | CCG | | GKSFSERSHLREHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆HKNALQNH | |
| | | | KCPECGKSFSHKNALQNHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QSSSLV | |
| | | | GEKPYKCPECGKSFSQSSSLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆TSGS | |
| | | | QRTHTGEKPYKCPECGKSFSTSG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆QS | |
| | | | SLVRHQRTHTGEKPYKCPECGKS | | GDLRRHX₁₇X₁₈X₁₉HX₂₀X₂₁X₂₂X₂₃X₂₄X₂₅X₂₆X₂₇X₂₈X₂₉X₃₀ | |
| | | | FSQSGDLRRHQRTHTGEKPTGKK | | | |
| | | | TS | | | |
| attagcacc | ATT AGC | 1036 | LEPGEKPYKCPECGKSFSERSHL | 1585 | X₁X₂X₃X₄X₅X₆X₇X₈X₉CX₁₀X₁₁CX₁₂X₁₃X₁₄X₁₅X₁₆ERSHLREHX₁₇X₁₈X₁₉ | 2134 |
| ccgggtgtc | ACC CCG | | REHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆DPGALVRHX₁₇X₁₈ | |
| agc | GGT GTC | | DPGALVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSGHLVRHX₁₇ | |
| | AGC | | GKSFSTSGHLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RNDTLTEH | |
| | | | KCPECGKSFSRNDTLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DKKDLT | |
| | | | GEKPYKCPECGKSFSDKKDLTRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆ERSH | |
| | | | QRTHTGEKPYKCPECGKSFSERS | | LREHX₁₇X₁₈X₁₉HX₂₀X₂₁X₂₂X₂₃X₂₄X₈X₉CX₁₀X₁₁CX₁₂X₁₃X₁₄X₁₅X₁₆HK | |
| | | | HLREHQRTHTGEKPYKCPECGKS | | NALQNHX₁₇X₁₈X₁₉HX₂₀X₂₁X₂₂X₂₃X₂₄X₂₅X₂₆X₂₇X₂₈X₂₉X₃₀ | |
| | | | FSHKNALQNHQRTHTGEKPTGKK | | | |
| | | | TS | | | |
| gtaattagc | GTA ATT | 1037 | LEPGEKPYKCPECGKSFSDPGAL | 1586 | X₁X₂X₃X₄X₅X₆X₇X₈X₉CX₁₀X₁₁CX₁₂X₁₃X₁₄X₁₅X₁₆DPGALVRHX₁₇X₁₈X₁₉ | 2135 |
| accccgggt | AGC ACC | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSGHLVRHX₁₇X₁₈ | |
| gtc | CCG GGT | | TSGHLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RNDTLTEHX₁₇ | |
| | GTC | | GKSFSRNDTLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DKKDLTRH | |
| | | | KCPECGKSFSDKKDLTRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆ERSHLR | |
| | | | GEKPYKCPECGKSFSERSHLREH | | EHX₁₇X₁₈X₁₉HX₂₀X₂₁X₂₂X₂₃X₂₄X₈X₉CX₁₀X₁₁CX₁₂X₁₃X₁₄X₁₅X₁₆HKNA | |
| | | | QRTHTGEKPYKCPECGKSFSHKN | | LQNHX₁₇X₁₈X₁₉HX₂₀X₂₁X₂₂X₂₃X₂₄X₈X₉CX₁₀X₁₁CX₁₂X₁₃X₁₄X₁₅X₁₆QS | |
| | | | ALQNHQRTHTGEKPYKCPECGKS | | SSLVRHX₁₇X₁₈X₁₉HX₂₀X₂₁X₂₂X₂₃X₂₄X₂₅X₂₆X₂₇X₂₈X₂₉X₃₀ | |
| | | | FSQSSSLVRHQRTHTGEKPTGKK | | | |
| | | | TS | | | |
| tgagttagg | TGA GTT | 1038 | LEPGEKPYKCPECGKSFSTSGSL | 1587 | X₁X₂X₃X₄X₅X₆X₇X₈X₉CX₁₀X₁₁CX₁₂X₁₃X₁₄X₁₅X₁₆TSGSLVRHX₁₇X₁₈X₁₉ | 2136 |
| gccccagca | AGG GCC | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QSGDLRRHX₁₇X₁₈ | |
| gtt | CCA GCA | | QSGDLRRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSHSLTEHX₁₇ | |
| | GTT | | GKSFSTSHSLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DCRDLARH | |
| | | | KCPECGKSFSDCRDLARHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDHLT | |
| | | | GEKPYKCPECGKSFSRSDHLTNH | | NHX₁₇X₁₈X₁₉HX₂₀X₂₁X₂₂X₂₃X₂₄X₈X₉CX₁₀X₁₁CX₁₂X₁₃X₁₄X₁₅X₁₆TSGS | |
| | | | QRTHTGEKPYKCPECGKSFSTSG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆QA | |
| | | | SLVRHQRTHTGEKPYKCPECGKS | | GHLASHX₁₇X₁₈X₁₉HX₂₀X₂₁X₂₂X₂₃X₂₄X₂₅X₂₆X₂₇X₂₈X₂₉X₃₀ | |
| | | | FSQAGHLASHQRTHTGEKPTGKK | | | |
| | | | TS | | | |
| gttagggcc | GTT AGG | 1039 | LEPGEKPYKCPECGKSFSQSSSL | 1588 | X₁X₂X₃X₄X₅X₆X₇X₈X₉CX₁₀X₁₁CX₁₂X₁₃X₁₄X₁₅X₁₆QSSSLVRHX₁₇X₁₈X₁₉ | 2137 |
| ccagcagtt | GCC CCA | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSGSLVRHX₁₇X₁₈ | |
| gta | GCA GTT | | TSGSLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QSGDLRRHX₁₇ | |
| | GTA | | GKSFSQSGDLRRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSHSLTEH | |
| | | | KCPECGKSFSTSHSLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DCRDLA | |
| | | | GEKPYKCPECGKSFSDCRDLARH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDH | |
| | | | QRTHTGEKPYKCPECGKSFSRSD | | LTNHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | HLTNHQRTHTGEKPYKCPECGKS | | GSLVRHX₁₇X₁₈X₁₉HX₂₀X₂₁X₂₂X₂₃X₂₄X₂₅X₂₆X₂₇X₂₈X₂₉X₃₀ | |
| | | | FSTSGSLVRHQRTHTGEKPTGKK | | | |
| | | | TS | | | |
| gttgtaatt | GTT GTA | 1040 | LEPGEKPYKCPECGKSFSTSGHL | 1589 | X₁X₂X₃X₄X₅X₆X₇X₈X₉CX₁₀X₁₁CX₁₂X₁₃X₁₄X₁₅X₁₆TSGHLVRHX₁₇X₁₈X₁₉ | 2138 |
| agcaccccg | ATT AGC | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RNDTLTEHX₁₇X₁₈ | |
| ggt | ACC CCG | | RNDTLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DKKDLTRHX₁₇ | |
| | GGT | | GKSFSDKKDLTRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆ERSHLREH | |
| | | | KCPECGKSFSERSHLREHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆HKNALQ | |
| | | | GEKPYKCPECGKSFSHKNALQNH | | NHX₁₇X₁₈X₁₉HX₂₀X₂₁X₂₂X₂₃X₂₄X₈X₉CX₁₀X₁₁CX₁₂X₁₃X₁₄X₁₅X₁₆QSSS | |
| | | | QRTHTGEKPYKCPECGKSFSQSS | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | SLVRHQRTHTGEKPYKCPECGKS | | GSLVRHX₁₇X₁₈X₁₉HX₂₀X₂₁X₂₂X₂₃X₂₄X₂₅X₂₆X₂₇X₂₈X₂₉X₃₀ | |
| | | | FSTSGSLVRHQRTHTGEKPTGKK | | | |
| | | | TS | | | |
| gtgagttag | gtg AGT | 1041 | LEPGEKPYKCPECGKSFSHRTTL | 1590 | X₁X₂X₃X₄X₅X₆X₇X₈X₉CX₁₀X₁₁CX₁₂X₁₃X₁₄X₁₅X₁₆HRTTLTNHX₁₇X₁₈X₁₉ | 2139 |
| ggccccagc | TAG GGC | | TNHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆ERSHLREHX₁₇X₁₈ | |
| agt | CCC AGC | | ERSHLREHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆SKKHLAEHX₁₇ | |
| | AGT | | GKSFSSKKHLAEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DPGHLVRH | |
| | | | KCPECGKSFSDPGHLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆REDNLH | |
| | | | GEKPYKCPECGKSFSREDNLHTH | | THX₁₇X₁₈X₁₉HX₂₀X₂₁X₂₂X₂₃X₂₄X₈X₉CX₁₀X₁₁CX₁₂X₁₃X₁₄X₁₅X₁₆HRTT | |
| | | | QRTHTGEKPYKCPECGKSFSHRT | | LTNHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | TLTNHQRTHTGEKPYKCPECGKS | | DELVRHX₁₇X₁₈X₁₉HX₂₀X₂₁X₂₂X₂₃X₂₄X₂₅X₂₆X₂₇X₂₈X₂₉X₃₀ | |
| | | | FSRSDELVRHQRTHTGEKPTGKK | | | |
| | | | TS | | | |
| cagccagaa | CAG CCA | 1042 | LEPGEKPYKCPECGKSFSRADNL | 1591 | X₁X₂X₃X₄X₅X₆X₇X₈X₉CX₁₀X₁₁CX₁₂X₁₃X₁₄X₁₅X₁₆RADNLTEHX₁₇X₁₈X₁₉ | 2140 |
| accaacaaa | GAA ACC | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QRANLRAHX₁₇X₁₈ | |
| cag | AAC AAA | | QRANLRAHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DSGNLRVHX₁₇ | |
| | CAG | | GKSFSDSGNLRVHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DKKDLTRH | |
| | | | KCPECGKSFSDKKDLTRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QSSNLV | |
| | | | GEKPYKCPECGKSFSQSSNLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆TSHS | |
| | | | QRTHTGEKPYKCPECGKSFSTSH | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆RA | |
| | | | SLTEHQRTHTGEKPYKCPECGKS | | DNLTEHX₁₇X₁₈X₁₉HX₂₀X₂₁X₂₂X₂₃X₂₄X₂₅X₂₆X₂₇X₂₈X₂₉X₃₀ | |
| | | | FSRADNLTEHQRTHTGEKPTGKK | | | |
| | | | TS | | | |
| gaaaccaac | GAA ACC | 1043 | LEPGEKPYKCPECGKSFSTTGNL | 1592 | X₁X₂X₃X₄X₅X₆X₇X₈X₉CX₁₀X₁₁CX₁₂X₁₃X₁₄X₁₅X₁₆TTGNLTVHX₁₇X₁₈X₁₉ | 2141 |
| aaacagcca | AAC AAA | | TVHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSHSLTEHX₁₇X₁₈ | |
| aat | CAG CCA | | TSHSLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RADNLTEHX₁₇ | |
| | AAT | | GKSFSRADNLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QRANLRAH | |
| | | | KCPECGKSFSQRANLRAHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DSGNLR | |
| | | | GEKPYKCPECGKSFSDSGNLRVH | | VHX₁₇X₁₈X₁₉HX₂₀X₂₁X₂₂X₂₃X₂₄X₈X₉CX₁₀X₁₁CX₁₂X₁₃X₁₄X₁₅X₁₆DKKD | |
| | | | QRTHTGEKPYKCPECGKSFSDKK | | LTRHX₁₇X₁₈X₁₉HX₂₀X₂₁X₂₂X₂₃X₂₄X₈X₉CX₁₀X₁₁CX₁₂X₁₃X₁₄X₁₅X₁₆QS | |
| | | | DLTRHQRTHTGEKPYKCPECGKS | | SNLVRHX₁₇X₁₈X₁₉HX₂₀X₂₁X₂₂X₂₃X₂₄X₂₅X₂₆X₂₇X₂₈X₂₉X₃₀ | |
| | | | FSQSSNLVRHQRTHTGEKPTGKK | | | |
| | | | TS | | | |
| ccagaaacc | CCA GAA | 1044 | LEPGEKPYKCPECGKSFSTSHSL | 1593 | X₁X₂X₃X₄X₅X₆X₇X₈X₉CX₁₀X₁₁CX₁₂X₁₃X₁₄X₁₅X₁₆TSHSLTEHX₁₇X₁₈X₁₉ | 2142 |
| aacaaacag | ACC AAC | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RADNLTEHX₁₇X₁₈ | |
| cca | AAA CAG | | RADNLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QRANLRAHX₁₇ | |
| | CCA | | GKSFSQRANLRAHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DSGNLRVH | |
| | | | KCPECGKSFSDSGNLRVHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DKKDLT | |
| | | | GEKPYKCPECGKSFSDKKDLTRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆QSSN | |
| | | | QRTHTGEKPYKCPECGKSFSQSS | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | NLVRHQRTHTGEKPYKCPECGKS | | HSLTEHX₁₇X₁₈X₁₉HX₂₀X₂₁X₂₂X₂₃X₂₄X₂₅X₂₆X₂₇X₂₈X₂₉X₃₀ | |
| | | | FSTSHSLTEHQRTHTGEKPTGKK | | | |
| | | | TS | | | |
| accaacaaa | ACC AAC | 1045 | LEPGEKPYKCPECGKSFSSKKHL | 1594 | X₁X₂X₃X₄X₅X₆X₇X₈X₉CX₁₀X₁₁CX₁₂X₁₃X₁₄X₁₅X₁₆SKKHLAEHX₁₇X₁₈X₁₉ | 2143 |
| cagccaaat | AAA CAG | | AEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TTGNLTVHX₁₇X₁₈ | |
| ccc | CCA AAT | | TTGNLTVHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSHSLTEHX₁₇ | |
| | CCC | | GKSFSTSHSLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RADNLTEH | |
| | | | KCPECGKSFSRADNLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QRANLR | |
| | | | GEKPYKCPECGKSFSQRANLRAH | | AHX₁₇X₁₈X₁₉HX₂₀X₂₁X₂₂X₂₃X₂₄X₈X₉CX₁₀X₁₁CX₁₂X₁₃X₁₄X₁₅X₁₆DSGN | |
| | | | QRTHTGEKPYKCPECGKSFSDSG | | LRVHX₁₇X₁₈X₁₉HX₂₀X₂₁X₂₂X₂₃X₂₄X₈X₉CX₁₀X₁₁CX₁₂X₁₃X₁₄X₁₅X₁₆DK | |
| | | | NLRVHQRTHTGEKPYKCPECGKS | | KDLTRHX₁₇X₁₈X₁₉HX₂₀X₂₁X₂₂X₂₃X₂₄X₂₅X₂₆X₂₇X₂₈X₂₉X₃₀ | |
| | | | FSDKKDLTRHQRTHTGEKPTGKK | | | |
| | | | TS | | | |
| gccagaaac | GCC AGA | 1046 | LEPGEKPYKCPECGKSFSDCRDL | 1595 | X₁X₂X₃X₄X₅X₆X₇X₈X₉CX₁₀X₁₁CX₁₂X₁₃X₁₄X₁₅X₁₆DCRDLARHX₁₇X₁₈X₁₉ | 2144 |
| caacaaaca | AAC CAA | | ARHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆SPADLTRHX₁₇X₁₈ | |
| gcc | CAA ACA | | SPADLTRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QSGNLTEHX₁₇ | |
| | GCC | | GKSFSQSGNLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QSGNLTEH | |
| | | | KCPECGKSFSQSGNLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DSGNLR | |
| | | | GEKPYKCPECGKSFSDSGNLRVH | | VHX₁₇X₁₈X₁₉HX₂₀X₂₁X₂₂X₂₃X₂₄X₈X₉CX₁₀X₁₁CX₁₂X₁₃X₁₄X₁₅X₁₆QLAH | |
| | | | QRTHTGEKPYKCPECGKSFSQLA | | LRAHX₁₇X₁₈X₁₉HX₂₀X₂₁X₂₂X₂₃X₂₄X₈X₉CX₁₀X₁₁CX₁₂X₁₃X₁₄X₁₅X₁₆DC | |
| | | | HLRAHQRTHTGEKPYKCPECGKS | | RDLARHX₁₇X₁₈X₁₉HX₂₀X₂₁X₂₂X₂₃X₂₄X₂₅X₂₆X₂₇X₂₈X₂₉X₃₀ | |
| | | | FSDCRDLARHQRTHTGEKPTGKK | | | |
| | | | TS | | | |
| agaaaccaa | AGA AAC | 1047 | LEPGEKPYKCPECGKSFSQRANL | 1596 | X₁X₂X₃X₄X₅X₆X₇X₈X₉CX₁₀X₁₁X₁₂X₁₃X₁₄X₁₅X₁₆QRANLRAHX₁₇X₁₈X₁₉ | 2145 |
| caaacagcc | CAA CAA | | RAHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆DCRDLARHX₁₇X₁₈ | |
| aaa | ACA GCC | | DCRDLARHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆SPADLTRHX₁₇ | |
| | AAA | | GKSFSSPADLTRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QSGNLTEH | |
| | | | KCPECGKSFSQSGNLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QSGNLT | |
| | | | GEKPYKCPECGKSFSQSGNLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆DSGN | |
| | | | QRTHTGEKPYKCPECGKSFSDSG | | LRVHX₁₇X₁₈X₁₉HX₂₀X₂₁X₂₂X₂₃X₂₄X₈X₉CX₁₀X₁₁CX₁₂X₁₃X₁₄X₁₅X₁₆QL | |
| | | | NLRVHQRTHTGEKPYKCPECGKS | | AHLRAHX₁₇X₁₈X₁₉HX₂₀X₂₁X₂₂X₂₃X₂₄X₂₅X₂₆X₂₇X₂₈X₂₉X₃₀ | |
| | | | FSQLAHLRAHQRTHTGEKPTGKK | | | |
| | | | TS | | | |
| cctgcagcc | CCT GCA | 138 | LEPGEKPYKCPECGKSFSQNSTL | 139 | X₁X₂X₃X₄X₅X₆X₇X₈X₉CX₁₀X₁₁CX₁₂X₁₃X₁₄X₁₅X₁₆QNSTLTEHX₁₇X₁₈X₁₉ | 322 |
| ccgcccagc | GCC CCG | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆ERSHLREHX₁₇X₁₈ | |
| cta | CCC AGC | | ERSHLREHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆SKKHLAEHX₁₇ | |
| | CTA | | GKSFSSKKHLAEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RNDTLTEH | |
| | | | KCPECGKSFSRNDTLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DCRDLA | |
| | | | GEKPYKCPECGKSFSDCRDLARH | | RHX₁₇X₁₈X₁₉HX₂₀X₂₁X₂₂X₂₃X₂₄X₈X₉CX₁₀X₁₁CX₁₂X₁₃X₁₄X₁₅X₁₆QSGD | |
| | | | QRTHTGEKPYKCPECGKSFSQSG | | LRRHX₁₇X₁₈X₁₉HX₂₀X₂₁X₂₂X₂₃X₂₄X₈X₉CX₁₀X₁₁CX₁₂X₁₃X₁₄X₁₅X₁₆TK | |
| | | | DLRRHQRTHTGEKPYKCPECGKS | | NSLTEHX₁₇X₁₈X₁₉HX₂₀X₂₁X₂₂X₂₃X₂₄X₂₅X₂₆X₂₇X₂₈X₂₉X₃₀ | |
| | | | FSTKNSLTEHQRTHTGEKPTGKK | | | |
| | | | TS | | | |
| accggcggg | ACC GGC | 1048 | LEPGEKPYKCPECGKSFSDSGNL | 1597 | X₁X₂X₃X₄X₅X₆X₇X₈X₉CX₁₀X₁₁CX₁₂X₁₃X₁₄X₁₅X₁₆DSGNLRVHX₁₇X₁₈X₁₉ | 2146 |
| ggaccgatt | GGG GGA | | RVHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆HKNALQNHX₁₇X₁₈ | |
| aac | CCG ATT | | HKNALQNHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RNDTLTEHX₁₇ | |
| | AAC | | GKSFSRNDTLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QRAHLERH | |
| | | | KCPECGKSFSQRAHLERHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDKLV | |
| | | | GEKPYKCPECGKSFSRSDKLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆DPGH | |
| | | | QRTHTGEKPYKCPECGKSFSDPG | | LVRHX₁₇X₁₈X₁₉HX₂₀X₂₁X₂₂X₂₃X₂₄X₈X₉CX₁₀X₁₁CX₁₂XEX₁₄X₁₅X₁₆DK | |
| | | | HLVRHQRTHTGEKPYKCPECGKS | | KDLTRHX₁₇X₁₈X₁₉HX₂₀X₂₁X₂₂X₂₃X₂₄X₂₅X₂₆X₂₇X₂₈X₂₉X₃₀ | |
| | | | FSDKKDLTRHQRTHTGEKPTGKK | | | |
| | | | TS | | | |
| ggcggggga | GGC GGG | 118 | LEPGEKPYKCPECGKSFSTSGNL | 119 | X₁X₂X₃X₄X₅X₆X₇X₈X₉CX₁₀X₁₁CX₁₂X₁₃X₁₄X₁₅X₁₆TSGNLTEHX₁₇X₁₈X₁₉ | 317 |
| ccgattaac | GGA CCG | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆DSGNLRVHX₁₇X₁₈ | |
| cat | ATT AAC | | DSGNLRVHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆HKNALQNHX₁₇ | |
| | CAT | | GKSFSHKNALQNHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RNDTLTEH | |
| | | | KCPECGKSFSRNDTLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QRAHLE | |
| | | | GEKPYKCPECGKSFSQRAHLERH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDK | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆DP | |
| | | | KLVRHQRTHTGEKPYKCPECGKS | | GHLVRHX₁₇X₁₈X₁₉HX₂₀X₂₁X₂₂X₂₃X₂₄X₂₅X₂₆X₂₇X₂₈X₂₉X₃₀ | |
| | | | FSDPGHLVRHQRTHTGEKPTGKK | | | |
| | | | TS | | | |
| cccacccct | CCC ACC | 142 | LEPGEKPYKCPECGKSFSERSHL | 143 | X₁X₂X₃X₄X₅X₆X₇X₈X₉CX₁₀X₁₁CX₁₂X₁₃X₁₄X₁₅X₁₆ERSHLREHX₁₇X₁₈X₁₉ | 323 |
| ccccggcag | CCT CCC | | REHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RADNLTEHX₁₇X₁₈ | |
| agc | CGG CAG | | RADNLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDKLTEHX₁₇ | |
| | AGC | | GKSFSRSDKLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆SKKHLAEH | |
| | | | KCPECGKSFSSKKHLAEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TKNSLT | |
| | | | GEKPYKCPECGKSFSTKNSLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆DKKD | |
| | | | QRTHTGEKPYKCPECGKSFSDKK | | LTRHX₁₇X₁₈X₁₉HX₂₀X₂₁X₂₂X₂₃X₂₄X₈X₉CX₁₀X₁₁CX₁₂X₁₃X₁₄X₁₅X₁₆SK | |
| | | | DLTRHQRTHTGEKPYKCPECGKS | | KHLAEHX₁₇X₁₈X₁₉HX₂₀X₂₁X₂₂X₂₃X₂₄X₂₅X₂₆X₂₇X₂₈X₂₉X₃₀ | |
| | | | FSSKKHLAEHQRTHTGEKPTGKK | | | |
| | | | TS | | | |
| acccccacc | ACC CCC | 1049 | LEPGEKPYKCPECGKSFSRADNL | 1598 | X₁X₂X₃X₄X₅X₆X₇X₈X₉CX₁₀X₁₁CX₁₂X₁₃X₁₄X₁₅X₁₆RADNLTEHX₁₇X₁₈X₁₉ | 2147 |
| cctccccgg | ACC CCT | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDKLTEHX₁₇X₁₈ | |
| cag | CCC CGG | | RSDKLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆SKKHLAEHX₁₇ | |
| | CAG | | GKSFSSKKHLAEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TKNSLTEH | |
| | | | KCPECGKSFSTKNSLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DKKDLT | |
| | | | GEKPYKCPECGKSFSDKKDLTRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆SKKH | |
| | | | QRTHTGEKPYKCPECGKSFSSKK | | LAEHX₁₇X₁₈X₁₉HX₂₀X₂₁X₂₂X₂₃X₂₄X₈X₉CX₁₀X₁₁CX₁₂X₁₃X₁₄X₁₅X₁₆DK | |
| | | | HLAEHQRTHTGEKPYKCPECGKS | | KDLTRHX₁₇X₁₈X₁₉HX₂₀X₂₁X₂₂X₂₃X₂₄X₂₅X₂₆X₂₇X₂₈X₂₉X₃₀ | |
| | | | FSDKKDLTRHQRTHTGEKPTGKK | | | |
| | | | TS | | | |
| cacagaggc | CAC AGA | 1050 | LEPGEKPYKCPECGKSFSTHLDL | 1599 | X₁X₂X₃X₄X₅X₆X₇X₈X₉CX₁₀X₁₁CX₁₂X₁₃X₁₄X₁₅X₁₆THLDLIRHX₁₇X₁₈X₁₉ | 2148 |
| taggccaag | GGC TAG | | IRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RKDNLKNHX₁₇X₁₈ | |
| act | GCC AAG | | RKDNLKNHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DCRDLARHX₁₇ | |
| | ACT | | GKSFSDCRDLARHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆REDNLHTH | |
| | | | KCPECGKSFSREDNLHTHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DPGHLV | |
| | | | GEKPYKCPECGKSFSDPGHLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆QLAH | |
| | | | QRTHTGEKPYKCPECGKSFSQLA | | LRAHX₁₇X₁₈X₁₉HX₂₀X₂₁X₂₂X₂₃X₂₄X₈X₉CX₁₀X₁₁CX₁₂X₁₃X₁₄X₁₅X₁₆SK | |
| | | | HLRAHQRTHTGEKPYKCPECGKS | | KALTEHX₁₇X₁₈X₁₉HX₂₀X₂₁X₂₂X₂₃X₂₄X₂₅X₂₆X₂₇X₂₈X₂₉X₃₀ | |
| | | | FSSKKALTEHQRTHTGEKPTGKK | | | |
| | | | TS | | | |
| agaggctag | AGA GGC | 146 | LEPGEKPYKCPECGKSFSSKKHL | 147 | X₁X₂X₃X₄X₅X₆X₇X₈X₉CX₁₀X₁₁CX₁₂X₁₃X₁₄X₁₅X₁₆SKKHLAEHX₁₇X₁₈X₁₉ | 324 |
| gccaagact | TAG GCC | | AEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆THLDLIRHX₁₇X₁₈ | |
| ccc | AAG ACT | | THLDLIRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RKDNLKNHX₁₇ | |
| | CCC | | GKSFSRKDNLKNHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DCRDLARH | |
| | | | KCPECGKSFSDCRDLARHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆REDNLH | |
| | | | GEKPYKCPECGKSFSREDNLHTH | | THX₁₇X₁₈X₁₉HX₂₀X₂₁X₂₂X₂₃X₂₄X₈X₉CX₁₀X₁₁CX₁₂X₁₃X₁₄X₁₅X₁₆DPGH | |
| | | | QRTHTGEKPYKCPECGKSFSDPG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆QL | |
| | | | HLVRHQRTHTGEKPYKCPECGKS | | AHLRAHX₁₇X₁₈X₁₉HX₂₀X₂₁X₂₂X₂₃X₂₄X₂₅X₂₆X₂₇X₂₈X₂₉X₃₀ | |
| | | | FSQLAHLRAHQRTHTGEKPTGKK | | | |
| | | | TS | | | |
| cttcacaga | CTT CAC | 1051 | LEPGEKPYKCPECGKSFSRKDNL | 1600 | X₁X₂X₃X₄X₅X₆X₇X₈X₉CX₁₀X₁₁CX₁₂X₁₃X₁₄X₁₅X₁₆RKDNLKNHX₁₇X₁₈X₁₉ | 2149 |
| ggctaggcc | AGA GGC | | KNHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆DCRDLARHX₁₇X₁₈ | |
| aag | TAG GCC | | DCRDLARHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆REDNLHTHX₁₇ | |
| | APG | | GKSFSREDNLHTHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DPGHLVRH | |
| | | | KCPECGKSFSDPGHLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QLAHLR | |
| | | | GEKPYKCPECGKSFSQLAHLRAH | | AHX₁₇X₁₈X₁₉HX₂₀X₂₁X₂₂X₂₃X₂₄X₈X₉CX₁₀X₁₁CX₁₂X₁₃X₁₄X₁₅X₁₆SKKA | |
| | | | QRTHTGEKPYKCPECGKSFSSKK | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆TT | |
| | | | ALTEHQRTHTGEKPYKCPECGKS | | GALTEHX₁₇X₁₈X₁₉HX₂₀X₂₁X₂₂X₂₃X₂₄X₂₅X₂₆X₂₇X₂₈X₂₉X₃₀ | |
| | | | FSTTGALTEHQRTHTGEKPTGKK | | | |
| | | | TS | | | |
| taggccaag | TAG GCC | 1052 | LEPGEKPYKCPECGKSFSQLAHL | 1601 | X₁X₂X₃X₄X₅X₆X₇X₈X₉CX₁₀X₁₁CX₁₂X₁₃X₁₄X₁₅X₁₆QLAHLRAHX₁₇X₁₈X₁₉ | 2150 |
| actcccagc | AAG ACT | | RAHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆ERSHLREHX₁₇X₁₈ | |
| aga | CCC AGC | | ERSHLREHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆SKKHLAEHX₁₇ | |
| | AGA | | GKSFSSKKHLAEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆THLDLIRH | |
| | | | KCPECGKSFSTHLDLIRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RKDNLK | |
| | | | GEKPYKCPECGKSFSRKDNLKNH | | NHX₁₇X₁₈X₁₉HX₂₀X₂₁X₂₂X₂₃X₂₄X₈X₉CX₁₀X₁₁CX₁₂X₁₃X₁₄X₁₅X₁₆DCRD | |
| | | | QRTHTGEKPYKCPECGKSFSDCR | | LARHX₁₇X₁₈X₁₉HX₂₀X₂₁X₂₂X₂₃X₂₄X₈X₉CX₁₀X₁₁CX₁₂X₁₃X₁₄X₁₅X₁₆RE | |
| | | | DLARHQRTHTGEKPYKCPECGKS | | DNLHTHX₁₇X₁₈X₁₉HX₂₀X₂₁X₂₂X₂₃X₂₄X₂₅X₂₆X₂₇X₂₈X₂₉X₃₀ | |
| | | | FSREDNLHTHQRTHTGEKPTGKK | | | |
| | | | TS | | | |
| ggctaggcc | GGC TAG | 1053 | LEPGEKPYKCPECGKSFSERSHL | 1602 | X₁X₂X₃X₄X₅X₆X₇X₈X₉CX₁₀X₁₁CX₁₂X₁₃X₁₄X₁₅X₁₆ERSHLREHX₁₇X₁₈X₁₉ | 2151 |
| aagactccc | GCC AAG | | REHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆SKKHLAEHX₁₇X₁₈ | |
| agc | ACT CCC | | SKKHLAEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆THLDLIRHX₁₇ | |
| | AGC | | GKSFSTHLDLIRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RKDNLKNH | |
| | | | KCPECGKSFSRKDNLKNHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DCRDLA | |
| | | | GEKPYKCPECGKSFSDCRDLARH | | RHX₁₇X₁₈X₁₉HX₂₀X₂₁X₂₂X₂₃X₂₄X₈X₉CX₁₀X₁₁CX₁₂X₁₃X₁₄X₁₅X₁₆REDN | |
| | | | QRTHTGEKPYKCPECGKSFSRED | | LHTHX₁₇X₁₈X₁₉HX₂₀X₂₁X₂₂X₂₃X₂₄X₈X₉CX₁₀X₁₁CX₁₂X₁₃X₁₄X₁₅X₁₆DP | |
| | | | NLHTHQRTHTGEKPYKCPECGKS | | GHLVRHX₁₇X₁₈X₁₉HX₂₀X₂₁X₂₂X₂₃X₂₄X₂₅X₂₆X₂₇X₂₈X₂₉X₃₀ | |
| | | | FSDPGHLVRHQRTHTGEKPTGKK | | | |
| | | | TS | | | |
| ccccttcac | CCC CTT | 1054 | LEPGEKPYKCPECGKSFSDCRDL | 1603 | X₁X₂X₃X₄X₅X₆X₇X₈X₉CX₁₀X₁₁CX₁₂X₁₃X₁₄X₁₅X₁₆DCRDLARHX₁₇X₁₈X₁₉ | 2152 |
| agaggctag | CAC AGA | | ARHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆REDNLHTHX₁₇X₁₈ | |
| gcc | GGC TAG | | REDNLHTHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DPGHLVRHX₁₇ | |
| | GCC | | GKSFSDPGHLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QLAHLRAH | |
| | | | KCPECGKSFSQLAHLRAHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆SKKALT | |
| | | | GEKPYKCPECGKSFSSKKALTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆TTGA | |
| | | | QRTHTGEKPYKCPECGKSFSTTG | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆SK | |
| | | | ALTEHQRTHTGEKPYKCPECGKS | | KHLAEHX₁₇X₁₈X₁₉HX₂₀X₂₁X₂₂X₂₃X₂₄X₂₅X₂₆X₂₇X₂₈X₂₉X₃₀ | |
| | | | FSSKKHLAEHQRTHTGEKPTGKK | | | |
| | | | TS | | | |
| ccacccctt | CCA CCC | 1055 | LEPGEKPYKCPECGKSFSREDNL | 1604 | X₁X₂X₃X₄X₅X₆X₇X₈X₉CX₁₀X₁₁CX₁₂X₁₃X₁₄X₁₅X₁₆REDNLHTHX₁₇X₁₈X₁₉ | 2153 |
| cacagaggc | CTT CAC | | HTHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆DPGHLVRHX₁₇X₁₈ | |
| tag | AGA GGC | | DPGHLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QLAHLRAHX₁₇ | |
| | TAG | | GKSFSQLAHLRAHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆SKKALTEH | |
| | | | KCPECGKSFSSKKALTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TTGALT | |
| | | | GEKPYKCPECGKSFSTTGALTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆SKKH | |
| | | | QRTHTGEKPYKCPECGKSFSSKK | | LAEHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | HLAEHQRTHTGEKPYKCPECGKS | | HSLTEHX₁₇X₁₈X₁₉HX₂₀X₂₁X₂₂X₂₃X₂₄X₂₅X₂₆X₂₇X₂₈X₂₉X₃₀ | |
| | | | FSTSHSLTEHQRTHTGEKPTGKK | | | |
| | | | TS | | | |
| cctccaccc | CCT CCA | 1056 | LEPGEKPYKCPECGKSFSDPGHL | 1605 | X₁X₂X₃X₄X₅X₆X₇X₈X₉CX₁₀X₁₁CX₁₂X₁₃X₁₄X₁₅X₁₆DPGHLVRHX₁₇X₁₈X₁₉ | 2154 |
| cttcacaga | CCC CTT | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QLAHLRAHX₁₇X₁₈ | |
| ggc | CAC AGA | | QLAHLRAHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆SKKALTEHX₁₇ | |
| | GGC | | GKSFSSKKALTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TTGALTEH | |
| | | | KCPECGKSFSTTGALTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆SKKHLA | |
| | | | GEKPYKCPECGKSFSSKKHLAEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆TSHS | |
| | | | QRTHTGEKPYKCPECGKSFSTSH | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆TK | |
| | | | SLTEHQRTHTGEKPYKCPECGKS | | NSLTEHX₁₇X₁₈X₁₉HX₂₀X₂₁X₂₂X₂₃X₂₄X₂₅X₂₆X₂₇X₂₈X₂₉X₃₀ | |
| | | | FSTKNSLTEHQRTHTGEKPTGKK | | | |
| | | | TS | | | |
| gcagagcct | GCA gag | 1057 | LEPGEKPYKCPECGKSFSSKKAL | 1606 | X₁X₂X₃X₄X₅X₆X₇X₈X₉CX₁₀X₁₁CX₁₂X₁₃X₁₄X₁₅X₁₆SKKALTEHX₁₇X₁₈X₁₉ | 2155 |
| ccacccctt | CCT CCA | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TTGALTEHX₁₇X₁₈ | |
| cac | CCC CTT | | TTGALTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆SKKHLAEHX₁₇ | |
| | CAC | | GKSFSSKKHLAEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSHSLTEH | |
| | | | KCPECGKSFSTSHSLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TKNSLT | |
| | | | GEKPYKCPECGKSFSTKNSLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆RSDN | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆QS | |
| | | | NLVRHQRTHTGEKPYKCPECGKS | | GDLRRHX₁₇X₁₈X₁₉HX₂₀X₂₁X₂₂X₂₃X₂₄X₂₅X₂₆X₂₇X₂₈X₂₉X₃₀ | |
| | | | FSQSGDLRRHQRTHTGEKPTGKK | | | |
| | | | TS | | | |
| gagcctcca | gag CCT | 1058 | LEPGEKPYKCPECGKSFSQLAHL | 1607 | X₁X₂X₃X₄X₅X₆X₇X₈X₉CX₁₀X₁₁CX₁₂X₁₃X₁₄X₁₅X₁₆QLAHLRAHX₁₇X₁₈X₁₉ | 2156 |
| ccccttcac | CCA CCC | | RAHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆SKKALTEHX₁₇X₁₈ | |
| aga | CTT CAC | | SKKALTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TTGALTEHX₁₇ | |
| | AGA | | GKSFSTTGALTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆SKKHLAEH | |
| | | | KCPECGKSFSSKKHLAEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSHSLT | |
| | | | GEKPYKCPECGKSFSTSHSLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆TKNS | |
| | | | QRTHTGEKPYKCPECGKSFSTKN | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | SLTEHQRTHTGEKPYKCPECGKS | | DNLVRHX₁₇X₁₈X₁₉HX₂₀X₂₁X₂₂X₂₃X₂₄X₂₅X₂₆X₂₇X₂₈X₂₉X₃₀ | |
| | | | FSRSDNLVRHQRTHTGEKPTGKK | | | |
| | | | TS | | | |
| ccggcagag | CCG GCA | 1059 | LEPGEKPYKCPECGKSFSTTGAL | 1608 | X₁X₂X₃X₄X₅X₆X₇X₈X₉CX₁₀X₁₁CX₁₂X₁₃X₁₄X₁₅X₁₆TTGALTEHX₁₇X₁₈X₁₉ | 2157 |
| cctccaccc | gag CCT | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆SKKHLAEHX₁₇X₁₈ | |
| ctt | CCA CCC | | SKKHLAEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSHSLTEHX₁₇ | |
| | CTT | | GKSFSTSHSLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TKNSLTEH | |
| | | | KCPECGKSFSTKNSLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDNLV | |
| | | | GEKPYKCPECGKSFSRSDNLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆QSGD | |
| | | | QRTHTGEKPYKCPECGKSFSQSG | | LRRHX₁₇X₁₈X₁₉HX₂₀X₂₁X₂₂X₂₃X₂₄X₈X₉CX₁₀X₁₁CX₁₂X₁₃X₁₄X₁₅X₁₆RN | |
| | | | DLRRHQRTHTGEKPYKCPECGKS | | DTLTEHX₁₇X₁₈X₁₉HX₂₀X₂₁X₂₂X₂₃X₂₄X₂₅X₂₆X₂₇X₂₈X₂₉X₃₀ | |
| | | | FSRNDTLTEHQRTHTGEKPTGKK | | | |
| | | | TS | | | |
| gcagatctt | GCA GAT | 1060 | LEPGEKPYKCPECGKSFSRSDKL | 1609 | X₁X₂X₃X₄X₅X₆X₇X₈X₉CX₁₀X₁₁CX₁₂X₁₃X₁₄X₁₅X₁₆RSDKLTEHX₁₇X₁₈X₁₉ | 2158 |
| cccagagga | CTT CCC | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QRAHLERHX₁₇X₁₈ | |
| cgg | AGA GGA | | QRAHLERHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QLAHLRAHX₁₇ | |
| | CGG | | GKSFSQLAHLRAHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆SKKHLAEH | |
| | | | KCPECGKSFSSKKHLAEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TTGALT | |
| | | | GEKPYKCPECGKSFSTTGALTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆TSGN | |
| | | | QRTHTGEKPYKCPECGKSFSTSG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆QS | |
| | | | NLVRHQRTHTGEKPYKCPECGKS | | GDLRRHX₁₇X₁₈X₁₉HX₂₀X₂₁X₂₂X₂₃X₂₄X₂₅X₂₆X₂₇X₂₈X₂₉X₃₀ | |
| | | | FSQSGDLRRHQRTHTGEKPTGKK | | | |
| | | | TS | | | |
| ccagcagat | CCA GCA | 114 | LEPGEKPYKCPECGKSFSQRAHL | 115 | X₁X₂X₃X₄X₅X₆X₇X₈X₉CX₁₀X₁₁CX₁₂X₁₃X₁₄X₁₅X₁₆QRAHLERHX₁₇X₁₈X₁₉ | 316 |
| cttcccaga | GAT CTT | | ERHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QLAHLRAHX₁₇X₁₈ | |
| gga | CCC AGA | | QLAHLRAHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆SKKHLAEHX₁₇ | |
| | GGA | | GKSFSSKKHLAEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TTGALTEH | |
| | | | KCPECGKSFSTTGALTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSGNLV | |
| | | | GEKPYKCPECGKSFSTSGNLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆QSGD | |
| | | | QRTHTGEKPYKCPECGKSFSQSG | | LRRHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | DLRRHQRTHTGEKPYKCPECGKS | | HSLTEHX₁₇X₁₈X₁₉HX₂₀X₂₁X₂₂X₂₃X₂₄X₂₅X₂₆X₂₇X₂₈X₂₉X₃₀ | |
| | | | FSTSHSLTEHQRTHTGEKPTGKK | | | |
| | | | TS | | | |
| ggcagagag | GGC AGA | 1061 | LEPGEKPYKCPECGKSFSRSDHL | 1610 | X₁X₂X₃X₄X₅X₆X₇X₈X₉CX₁₀X₁₁CX₁₂X₁₃X₁₄X₁₅X₁₆RSDHLTNHX₁₇X₁₈X₁₉ | 2159 |
| ggcactggg | GAG GGC | | TNHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDKLVRHX₁₇X₁₈ | |
| agg | ACT ggg | | RSDKLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆THLDLIRHX₁₇ | |
| | AGG | | GKSFSTHLDIIRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DPGHLVRH | |
| | | | KCPECGKSFSDPGHLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDNLV | |
| | | | GEKPYKCPECGKSFSRSDNLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆QLAH | |
| | | | QRTHTGEKPYKCPECGKSFSQLA | | LRAHX₁₇X₁₈X₁₉HX₂₀X₂₁X₂₂X₂₃X₂₄X₈X₉CX₁₀X₁₁CX₁₂X₁₃X₁₄X₁₅X₁₆DP | |
| | | | HLRAHQRTHTGEKPYKCPECGKS | | GHLVRHX₁₇X₁₈X₁₉HX₂₀X₂₁X₂₂X₂₃X₂₄X₂₅X₂₆X₂₇X₂₈X₂₉X₃₀ | |
| | | | FSDPGHLVRHQRTHTGEKPTGKK | | | |
| | | | TS | | | |
| gaaggcaga | GAA GGC | 1062 | LEPGEKPYKCPECGKSFSRSDKL | 1611 | X₁X₂X₃X₄X₅X₆X₇X₈X₉CX₁₀X₁₁CX₁₂X₁₃X₁₄X₁₅X₁₆RSDKLVRHX₁₇X₁₈X₁₉ | 2160 |
| gagggcact | AGA GAG | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆THLDLIRHX₁₇X₁₈ | |
| ggg | GGC ACT | | THLDLIRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DPGHLVRHX₁₇ | |
| | GGG | | GKSFSDPGHLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDNLVRH | |
| | | | KCPECGKSFSRSDNLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QLAHLR | |
| | | | GEKPYKCPECGKSFSQLAHLRAH | | AHX₁₇X₁₈X₁₉HX₂₀X₂₁X₂₂X₂₃X₂₄X₈X₉CX₁₀X₁₁CX₁₂X₁₃X₁₄X₁₅X₁₆DPGH | |
| | | | QRTHTGEKPYKCPECGKSFSDPG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆QS | |
| | | | HLVRHQRTHTGEKPYKCPECGKS | | SNLVRHX₁₇X₁₈X₁₉HX₂₀X₂₁X₂₂X₂₃X₂₄X₂₅X₂₆X₂₇X₂₈X₂₉X₃₀ | |
| | | | FSQSSNLVRHQRTHTGEKPTGKK | | | |
| | | | TS | | | |
| gagggcact | GAG GGC | 1063 | LEPGEKPYKCPECGKSFSRADNL | 1612 | X₁X₂X₃X₄X₅X₆X₇X₈X₉CX₁₀X₁₁CX₁₂X₁₃X₁₄X₁₅X₁₆RADNLTEHX₁₇X₁₈X₁₉ | 2161 |
| gggaggagg | ACT ggg | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDHLTNHX₁₇X₁₈ | |
| cag | AGG AGG | | RSDHLTNHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDHLTNHX₁₇ | |
| | CAG | | GKSFSRSDHLTNHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDKLVRH | |
| | | | KCPECGKSFSRSDKLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆THLDLI | |
| | | | GEKPYKCPECGKSFSTHLDLIRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆DPGH | |
| | | | QRTHTGEKPYKCPECGKSFSDPG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | HLVRHQRTHTGEKPYKCPECGKS | | DNLVRHX₁₇X₁₈X₁₉HX₂₀X₂₁X₂₂X₂₃X₂₄X₂₅X₂₆X₂₇X₂₈X₂₉X₃₀ | |
| | | | FSRSDNLVRHQRTHTGEKPTGKK | | | |
| | | | TS | | | |
| agagagggc | AGA GAG | 1064 | LEPGEKPYKCPECGKSFSRSDHL | 1613 | X₁X₂X₃X₄X₅X₆X₇X₈X₉CX₁₀X₁₁CX₁₂X₁₃X₁₄X₁₅X₁₆RSDHLTNHX₁₇X₁₈X₁₉ | 2162 |
| actgggagg | GGC ACT | | TNHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDHLTNHX₁₇X₁₈ | |
| agg | ggg AGG | | RSDHLTNHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDKLVRHX₁₇ | |
| | AGG | | GKSFSRSDKLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆THLDLIRH | |
| | | | KCPECGKSFSTHLDLIRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DPGHLV | |
| | | | GEKPYKCPECGKSFSDPGHLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDN | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆QL | |
| | | | NLVRHQRTHTGEKPYKCPECGKS | | AHLRAHX₁₇X₁₈X₁₉HX₂₀X₂₁X₂₂X₂₃X₂₄X₂₅X₂₆X₂₇X₂₈X₂₉X₃₀ | |
| | | | FSQLAHLRAHQRTHTGEKPTGKK | | | |
| | | | TS | | | |
| gggaggagg | ggg AGG | 1065 | LEPGEKPYKCPECGKSFSDPGHL | 1614 | X₁X₂X₃X₄X₅X₆X₇X₈X₉CX₁₀X₁₁CX₁₂X₁₃X₁₄X₁₅X₁₆DPGHLVRHX₁₇X₁₈X₁₉ | 2163 |
| cagtgggag | AGG CAG | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDNLVRHX₁₇X₁₈ | |
| ggc | TGG GAG | | RSDNLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDHLTTHX₁₇ | |
| | GGC | | GKSFSRSDHLTTHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RADNLTEH | |
| | | | KCPECGKSFSRADNLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDHLT | |
| | | | GEKPYKCPECGKSFSRSDHLTNH | | NHX₁₇X₁₈X₁₉HX₂₀X₂₁X₂₂X₂₃X₂₄X₈X₉CX₁₀X₁₁CX₁₂X₁₃X₁₄X₁₅X₁₆RSDH | |
| | | | QRTHTGEKPYKCPECGKSFSRSD | | LTNHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | HLTNHQRTHTGEKPYKCPECGKS | | DKLVRHX₁₇X₁₈KX₁₉HX₂₀X₂₁X₂₂X₂₃X₂₄X₂₅X₂₆X₂₇X₂₈X₂₉X₃₀ | |
| | | | FSRSDKLVRHQRTHTGEKPTGKK | | | |
| | | | TS | | | |
| ggcactggg | GGC ACT | 1066 | LEPGEKPYKCPECGKSFSRSDHL | 1615 | X₁X₂X₃X₄X₅X₆X₇X₈X₉CX₁₀X₁₁CX₁₂X₁₃X₁₄X₁₅X₁₆RSDHLTTHX₁₇X₁₈X₁₉ | 2164 |
| aggaggcag | ggg AGG | | TTHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RADNLTEHX₁₇X₁₈ | |
| tgg | AGG CAG | | RADNLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDHLTNHX₁₇ | |
| | TGG | | GKSFSRSDHLTNHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDHLTNH | |
| | | | KCPECGKSFSRSDHLTNHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDKLV | |
| | | | GEKPYKCPECGKSFSRSDKLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆THLD | |
| | | | QRTHTGEKPYKCPECGKSFSTHL | | LIRHX₁₇X₁₈X₁₉HX₂₀X₂₁X₂₂X₂₃X₂₄X₈X₉CX₁₀X₁₁CX₁₂X₁₃X₁₄X₁₅X₁₆DP | |
| | | | DLIRHQRTHTGEKPYKCPECGKS | | GHLVRHX₁₇X₁₈X₁₉HX₂₀X₂₁X₂₂X₂₃X₂₄X₂₅X₂₆X₂₇X₂₈X₂₉X₃₀ | |
| | | | FSDPGHLVRHQRTHTGEKPTGKK | | | |
| | | | TS | | | |
| gagggcgga | GAG GGC | 1067 | LEPGEKPYKCPECGKSFSTKNSL | 1616 | X₁X₂X₃X₄X₅X₆X₇X₈X₉CX₁₀X₁₁CX₁₂X₁₃X₁₄X₁₅X₁₆TKNSLTEHX₁₇X₁₈X₁₉ | 2165 |
| gggcggggg | GGA ggg | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDKLVRHX₁₇X₁₈ | |
| cct | CGG ggg | | RSDKLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDKLTEHX₁₇ | |
| | CCT | | GKSFSRSDKLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDKLVRH | |
| | | | KCPECGKSFSRSDKLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QRAHLE | |
| | | | GEKPYKCPECGKSFSQRAHLERH | | RHX₁₇X₁₈X₁₉HX₂₀X₂₁X₂₂X₂₃X₂₄X₈X₉CX₁₀X₁₁CX₁₂X₁₃X₁₄X₁₅X₁₆DPGH | |
| | | | QRTHTGEKPYKCPECGKSFSDPG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | HLVRHQRTHTGEKPYKCPECGKS | | DNLVRHX₁₇X₁₈X₁₉HX₂₀X₂₁X₂₂X₂₃X₂₄X₂₅X₂₆X₂₇X₂₈X₂₉X₃₀ | |
| | | | FSRSDNLVRHQRTHTGEKPTGKK | | | |
| | | | TS | | | |
| actgggagg | ACT ggg | 1068 | LEPGEKPYKCPECGKSFSRSDNL | 1617 | X₁X₂X₃X₄X₅X₆X₇X₈X₉CX₁₀X₁₁CX₁₂X₁₃X₁₄X₁₅X₁₆RSDNLVRHX₁₇X₁₈X₁₉ | 2166 |
| aggcagtgg | AGG AGG | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDHLTTHX₁₇X₁₈ | |
| gag | CAG TGG | | RSDHLTTHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RADNLTEHX₁₇ | |
| | GAG | | GKSFSRADNLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDHLTNH | |
| | | | KCPECGKSFSRSDHLTNHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDHLT | |
| | | | GEKPYKCPECGKSFSRSDHLTNH | | NHX₁₇X₁₈X₁₉HX₂₀X₂₁X₂₂X₂₃X₂₄X₈X₉CX₁₀X₁₁CX₁₂X₁₃X₁₄X₁₅X₁₆RSDK | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆TH | |
| | | | KLVRHQRTHTGEKPYKCPECGKS | | LDLIRHX₁₇X₁₈X₁₉HX₂₀X₂₁X₂₂X₂₃X₂₄X₂₅X₂₆X₂₇X₂₈X₂₉X₃₀ | |
| | | | FSTHLDLIRHQRTHTGEKPTGKK | | | |
| | | | TS | | | |
| tgaaaggaa | TGA AAG | 1069 | LEPGEKPYKCPECGKSFSDPGHL | 1618 | X₁X₂X₃X₄X₅X₆X₇X₈X₉CX₁₀X₁₁CX₁₂X₁₃X₁₄X₁₅X₁₆DPGHLVRHX₁₇X₁₈X₁₉ | 2167 |
| ggcagagag | GAA GGC | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDNLVRHX₁₇X₁₈ | |
| ggc | AGA GAG | | RSDNLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QLAHLRAHX₁₇ | |
| | GGC | | GKSFSQLAHLRAHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DPGHLVRH | |
| | | | KCPECGKSFSDPGHLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QSSNLV | |
| | | | GEKPYKCPECGKSFSQSSNLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RKDN | |
| | | | QRTHTGEKPYKCPECGKSFSRKD | | LKNHX₁₇X₁₈X₁₉HX₂₀X₂₁X₂₂X₂₃X₂₄X₈X₉CX₁₀X₁₁CX₁₂X₁₃X₁₄X₁₅X₁₆QA | |
| | | | NLKNHQRTHTGEKPYKCPECGKS | | GHLASHX₁₇X₁₈X₁₉HX₂₀X₂₁X₂₂X₂₃X₂₄X₂₅X₂₆X₂₇X₂₈X₂₉X₃₀ | |
| | | | FSQAGHLASHQRTHTGEKPTGKK | | | |
| | | | TS | | | |
| aaggaaggc | AAG GAA | 1070 | LEPGEKPYKCPECGKSFSTHLDL | 1619 | X₁X₂X₃X₄X₅X₆X₇X₈X₉CX₁₀X₁₁CX₁₂X₁₃X₁₄X₁₅X₁₆THLDLIRHX₁₇X₁₈X₁₉ | 2168 |
| agagagggc | GGC AGA | | IRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆DPGHLVRHX₁₇X₁₈ | |
| act | GAG GGC | | DPGHLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDNLVRHX₁₇ | |
| | ACT | | GKSFSRSDNLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QLAHLRAH | |
| | | | KCPECGKSFSQLAHLRAHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DPGHLV | |
| | | | GEKPYKCPECGKSFSDPGHLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆QSSN | |
| | | | QRTHTGEKPYKCPECGKSFSQSS | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RK | |
| | | | NLVRHQRTHTGEKPYKCPECGKS | | DNLKNHX₁₇X₁₈X₁₉HX₂₀X₂₁X₂₂X₂₃X₂₄X₂₅X₂₆X₂₇X₂₈X₂₉X₃₀ | |
| | | | FSRKDNLKNHQRTHTGEKPTGKK | | | |
| | | | TS | | | |
| tgggagggc | TGG GAG | 1071 | LEPGEKPYKCPECGKSFSRSDKL | 1620 | X₁X₂X₃X₄X₅X₆X₇X₈X₉CX₁₀X₁₁CX₁₂X₁₃X₁₄X₁₅X₁₆RSDKLVRHX₁₇X₁₈X₁₉ | 2169 |
| ggagggcgg | GGC GGA | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDKLTEHX₁₇X₁₈ | |
| ggg | ggg CGG | | RSDKLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDKLVRHX₁₇ | |
| | GGG | | GKSFSRSDKLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QRAHLERH | |
| | | | KCPECGKSFSQRAHLERHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DPGHLV | |
| | | | GEKPYKCPECGKSFSDPGHLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDN | |
| | | | QRTHTGEKPYKCPECGKSFSRSD | | VLVRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | NLVRHQRTHTGEKPYKCPECGKS | | DHLTTHX₁₇X₁₈X₁₉HX₂₀X₂₁X₂₂X₂₃X₂₄X₂₅X₂₆X₂₇X₂₈X₂₉X₃₀ | |
| | | | FSRSDHLTTHQRTHTGEKPTGKK | | | |
| | | | TS | | | |
| cagtgggag | CAG TGG | 1072 | LEPGEKPYKCPECGKSFSRSDKL | 1621 | X₁X₂X₃X₄X₅X₆X₇X₈X₉CX₁₀X₁₁CX₁₂X₁₃X₁₄X₁₅X₁₆RSDKLTEHX₁₇X₁₈X₁₉ | 2170 |
| ggcggaggg | GAG GGC | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDKLVRHX₁₇X₁₈ | |
| cgg | GGA ggg | | RSDKLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QRAHLERHX₁₇ | |
| | CGG | | GKSFSQRAHLERHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DPGHLVRH | |
| | | | KCPECGKSFSDPGHLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDNLV | |
| | | | GEKPYKCPECGKSFSRSDNLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDH | |
| | | | QRTHTGEKPYKCPECGKSFSRSD | | LTTHX₁₇X₁₈X₁₉HX₂₀X₂₁X₂₂X₂₃X₂₄X₈X₉CX₁₀X₁₁CX₁₂X₁₃X₁₄X₁₅X₁₆RA | |
| | | | HLTTHQRTHTGEKPYKCPECGKS | | DNLTEHX₁₇X₁₈X₁₉HX₂₀X₂₁X₂₂X₂₃X₂₄X₂₅X₂₆X₂₇X₂₈X₂₉X₃₀ | |
| | | | FSRADNLTEHQRTHTGEKPTGKK | | | |
| | | | TS | | | |
| aggcagtgg | AGG CAG | 1073 | LEPGEKPYKCPECGKSFSRSDKL | 1622 | X₁X₂X₃X₄X₅X₆X₇X₈X₉CX₁₀X₁₁CX₁₂X₁₃X₁₄X₁₅X₁₆RSDKLVRHX₁₇X₁₈X₁₉ | 2171 |
| gagggcgga | TGG GAG | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QRAHLERHX₁₇X₁₈ | |
| ggg | GGC GGA | | QRAHLERHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DPGHLVRHX₁₇ | |
| | GGG | | GKSFSDPGHLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDNLVRH | |
| | | | KCPECGKSFSRSDNLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDHLT | |
| | | | GEKPYKCPECGKSFSRSDHLTTH | | THX₁₇X₁₈X₁₉HX₂₀X₂₁X₂₂X₂₃X₂₄X₈X₉CX₁₀X₁₁CX₁₂X₁₃X₁₄X₁₅X₁₆RADN | |
| | | | QRTHTGEKPYKCPECGKSFSRAD | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | NLTEHQRTHTGEKPYKCPECGKS | | DHLTNHX₁₇X₁₈X₁₉HX₂₀X₂₁X₂₂X₂₃X₂₄X₂₅X₂₆X₂₇X₂₈X₂₉X₃₀ | |
| | | | FSRSDHLTNHQRTHTGEKPTGKK | | | |
| | | | TS | | | |
| aggaggcag | AGG AGG | 1074 | LEPGEKPYKCPECGKSFSQRAHL | 1623 | X₁X₂X₃X₄X₅X₆X₇X₈X₉CX₁₀X₁₁CX₁₂X₁₃X₁₄X₁₅X₁₆QRAHLERHX₁₇X₁₈X₁₉ | 2172 |
| tgggagggc | CAG TGG | | ERHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆DPGHLVRHX₁₇X₁₈ | |
| gga | GAG GGC | | DPGHLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDNLVRHX₁₇ | |
| | GGA | | GKSFSRSDNLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDHLTTH | |
| | | | KCPECGKSFSRSDHLTTHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RADNLT | |
| | | | GEKPYKCPECGKSFSRADNLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆RSDH | |
| | | | QRTHTGEKPYKCPECGKSFSRSD | | LTNHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | HLTNHQRTHTGEKPYKCPECGKS | | DHLTNHX₁₇X₁₈X₁₉HX₂₀X₂₁X₂₂X₂₃X₂₄X₂₅X₂₆X₂₇X₂₈X₂₉X₃₀ | |
| | | | FSRSDHLTNHQRTHTGEKPTGKK | | | |
| | | | TS | | | |
| gtttgaaag | GTT TGA | 110 | LEPGEKPYKCPECGKSFSRSDNL | 111 | X₁X₂X₃X₄X₅X₆X₇X₈X₉CX₁₀X₁₁CX₁₂X₁₃X₁₄X₁₅X₁₆RSDNLVRHX₁₇X₁₈X₁₉ | 315 |
| gaaggcaga | AAG GAA | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QLAHLRAHX₁₇X₁₈ | |
| gag | GGC AGA | | QLAHLRAHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DPGHLVRHX₁₇ | |
| | GAG | | GKSFSDPGHLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QSSNLVRH | |
| | | | KCPECGKSFSQSSNLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RKDNLK | |
| | | | GEKPYKCPECGKSFSRKDNLKNH | | NHX₁₇X₁₈X₁₉HX₂₀X₂₁X₂₂X₂₃X₂₄X₈X₉CX₁₀X₁₁CX₁₂X₁₃X₁₄X₁₅X₁₆QAGH | |
| | | | QRTHTGEKPYKCPECGKSFSQAG | | LASHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | HLASHQRTHTGEKPYKCPECGKS | | GSLVRHX₁₇X₁₈X₁₉HX₂₀X₂₁X₂₂X₂₃X₂₄X₂₅X₂₆X₂₇X₂₈X₂₉X₃₀ | |
| | | | FVSTSGSLVRHQRTHTGEKPTGK | | | |
| | | | TS | | | |
| acggtttga | ACG GTT | 1075 | LEPGEKPYKCPECGKSFSQLAHL | 1624 | X₁X₂X₃X₄X₅X₆X₇X₈X₉CX₁₀X₁₁CX₁₂X₁₃X₁₄X₁₅X₁₆QLAHLRAHX₁₇X₁₈X₁₉ | 2173 |
| aaggaaggc | TGA AAG | | RAHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆DPGHLVRHX₁₇X₁₈ | |
| aga | GAA GGC | | DPGHLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QSSNLVRHX₁₇ | |
| | AGA | | GKSFSQSSNLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RKDNLKNH | |
| | | | KCPECGKSFSRKDNLKNHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QAGHLA | |
| | | | GEKPYKCPECGKSFSQAGHLASH | | SHX₁₇X₁₈X₁₉HX₂₀X₂₁X₂₂X₂₃X₂₄X₈X₉CX₁₀X₁₁CX₁₂X₁₃X₁₄X₁₅X₁₆TSGS | |
| | | | QRTHTGEKPYKCPECGKSFSTSG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RT | |
| | | | SLVRHQRTHTGEKPYKCPECGKS | | DTLRDHX₁₇X₁₈X₁₉HX₂₀X₂₁X₂₂X₂₃X₂₄X₂₅X₂₆X₂₇X₂₈X₂₉X₃₀ | |
| | | | FSRTDTLRDHQRTHTGEKPTGKK | | | |
| | | | TS | | | |
| aggacggtt | AGG ACG | 1076 | LEPGEKPYKCPECGKSFSDPGHL | 1625 | X₁X₂X₃X₄X₅X₆X₇X₈X₉CX₁₀X₁₁CX₁₂X₁₃X₁₄X₁₅X₁₆DPGHLVRHX₁₇X₁₈X₁₉ | 2174 |
| tgaaaggaa | GTT TGA | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QSSNLVRHX₁₇X₁₈ | |
| ggc | AAG GAA | | QSSNLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RKDNLKNHX₁₇ | |
| | GGC | | GKSFSRKDNLKNHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QAGHLASH | |
| | | | KCPECGKSFSQAGHLASHQRTHT | | X₁₇X₁₈X₁₉HX₂₀X₂₁X₂₂X₂₃X₂₄X₈X₉CXHX₁₁CX₁₂X₁₃X₁₄X₁₅X₁₆TSGSLV | |
| | | | GEKPYKCPECGKSFSTSGSLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RTDT | |
| | | | QRTHTGEKPYKCPECGKSFSRTD | | LRDHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | TLRDHQRTHTGEKPYKCPECGKS | | DHLTNHX₁₇X₁₈X₁₉HX₂₀X₂₁X₂₂X₂₃X₂₄X₂₅X₂₆X₂₇X₂₈X₂₉X₃₀ | |
| | | | FSRSDHLTNHQRTHTGEKPTGKK | | | |
| | | | TS | | | |
| cagaggacg | CAG AGG | 1077 | LEPGEKPYKCPECGKSFSQSSNL | 1626 | X₁X₂X₃X₄X₅X₆X₇X₈X₉CX₁₀X₁₁CX₁₂X₁₃X₁₄X₁₅X₁₆QSSNLVRHX₁₇X₁₈X₁₉ | 2175 |
| gtttgaaag | ACG GTT | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RKDNLKNHX₁₇X₁₈ | |
| gaa | TGA AAG | | RKDNLKNHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QAGHLASHX₁₇ | |
| | GAA | | GKSFSQAGHLASHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSGSLVRH | |
| | | | KCPECGKSFSTSGSLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RTDTLR | |
| | | | GEKPYKCPECGKSFSRTDTLRDH | | DHX₁₇X₁₈X₁₉HX₂₀X₂₁X₂₂X₂₃X₂₄X₈X₉CX₁₀X₁₁CX₁₂X₁₃X₁₄X₁₅X₁₆RSDH | |
| | | | QRTHTGEKPYKCPECGKSFSRSD | | LTNHX₁₇X₁₈X₁₉HX₂₀X₂₁X₂₂X₂₃X₂₄X₈X₉CX₁₀X₁₁CX₁₂X₁₃X₁₄X₁₅X₁₆RA | |
| | | | HLTNHQRTHTGEKPYKCPECGKS | | DNLTEHX₁₇X₁₈X₁₉HX₂₀X₂₁X₂₂X₂₃X₂₄X₂₅X₂₆X₂₇X₂₈X₂₉X₃₀ | |
| | | | FSRADNLTEHQRTHTGEKPTGKK | | | |
| | | | TS | | | |
| gggcgccca | ggg CGC | 1078 | LEPGEKPYKCPECGKSFSRSDHL | 1627 | X₁X₂X₃X₄X₅X₆X₇X₈X₉CX₁₀X₁₁CX₁₂X₁₃X₁₄X₁₅X₁₆RSDHLTNHX₁₇X₁₈X₁₉ | 2176 |
| gggtagggc | CCA GGG | | TNHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆DPGHLVRHX₁₇X₁₈ | |
| agg | TAG GGC | | DPGHLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆REDNLHTHX₁₇ | |
| | AGG | | GKSFSREDNLHTHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDKLVRH | |
| | | | KCPECGKSFSRSDKLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSHSLT | |
| | | | GEKPYKCPECGKSFSTSHSLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆HTGH | |
| | | | QRTHTGEKPYKCPECGKSFSHTG | | LLEHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | HLLEHQRTHTGEKPYKCPECGKS | | DKLVRHX₁₇X₁₈X₁₉HX₂₀X₂₁X₂₂X₂₃X₂₄X₂₅X₂₆X₂₇X₂₈X₂₉X₃₀ | |
| | | | FSRSDKLVRHQRTHTGEKPTGKK | | | |
| | | | TS | | | |
| cgcccaggg | CGC CCA | 1079 | LEPGEKPYKCPECGKSFSRSDHL | 1628 | X₁X₂X₃X₄X₅X₆X₇X₈X₉CX₁₀X₁₁CX₁₂X₁₃X₁₄X₁₅X₁₆RSDHLTTHX₁₇X₁₈X₁₉ | 2177 |
| tagggcagg | GGG TAG | | TTHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDHLTNHX₁₇X₁₈ | |
| tgg | GGC AGG | | RSDHLTNHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DPGHLVRHX₁₇ | |
| | TGG | | GKSFSDPGHLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆REDNLHTH | |
| | | | KCPECGKSFSREDNLHTHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDKLV | |
| | | | GEKPYKCPECGKSFSRSDKLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆TSHS | |
| | | | QRTHTGEKPYKCPECGKSFSTSH | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆HT | |
| | | | SLTEHQRTHTGEKPYKCPECGKS | | GHLLEHX₁₇X₁₈X₁₉HX₂₀X₂₁X₂₂X₂₃X₂₄X₂₅X₂₆X₂₇X₂₈X₂₉X₃₀ | |
| | | | FSHTGHLLEHQRTHTGEKPTGKK | | | |
| | | | TS | | | |
| gggagggcg | ggg AGG | 1080 | LEPGEKPYKCPECGKSFSDPGHL | 1629 | X₁X₂X₃X₄X₅X₆X₇X₈X₉CX₁₀X₁₁CX₁₂X₁₃X₁₄X₁₅X₁₆DPGHLVRHX₁₇X₁₈X₁₉ | 2178 |
| gagggcggg | GCG GAG | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDKLVRHX₁₇X₁₈ | |
| ggc | GGC GGG | | RSDKLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DPGHLVRHX₁₇ | |
| | GGC | | GKSFSDPGHLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDNLVRH | |
| | | | KCPECGKSFSRSDNLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDDLV | |
| | | | GEKPYKCPECGKSFSRSDDLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDH | |
| | | | QRTHTGEKPYKCPECGKSFSRSD | | LTNHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | HLTNHQRTHTGEKPYKCPECGKS | | DKLVRHX₁₇X₁₈X₁₉HX₂₀X₂₁X₂₂X₂₃X₂₄X₂₅X₂₆X₂₇X₂₈X₂₉X₃₀ | |
| | | | FSRSDKLVRHQRTHTGEKPTGKK | | | |
| | | | TS | | | |
| cggcgtgga | CGG CGT | 1081 | LEPGEKPYKCPECGKSFSTTGNL | 1630 | X₁X₂X₃X₄X₅X₆X₇X₈X₉CX₁₀X₁₁CX₁₂X₁₃X₁₄X₁₅X₁₆TTGNLTVHX₁₇X₁₈X₁₉ | 2179 |
| ggcagggag | GGA GGC | | TVHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDNLVRHX₁₇X₁₈ | |
| aat | AGG gag | | RSDNLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDHLTNHX₁₇ | |
| | AAT | | GKSFSRSDHLTNHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DPGHLVRH | |
| | | | KCPECGKSFSDPGHLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QRAHLE | |
| | | | GEKPYKCPECGKSFSQRAHLERH | | RHX₁₇X₁₈X₁₉HX₂₀X₂₁X₂₂X₂₃X₂₄X₈X₉CX₁₀X₁₁CX₁₂X₁₃X₁₄X₁₅X₁₆SRRT | |
| | | | QRTHTGEKPYKCPECGKSFSSRR | | CRAHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | TCRAHQRTHTGEKPYKCPECGKS | | DKLTEHX₁₇X₁₈X₁₉HX₂₀X₂₁X₂₂X₂₃X₂₄X₂₅X₂₆X₂₇X₂₈X₂₉X₃₀ | |
| | | | FSRSDKLTEHQRTHTGEKPTGKK | | | |
| | | | TS | | | |
| aggaaggca | AGG AAG | 1082 | LEPGEKPYKCPECGKSFSRNDAL | 1631 | X₁X₂X₃X₄X₅X₆X₇X₈X₉CX₁₀X₁₁CX₁₂X₁₃X₁₄X₁₅X₁₆RNDALTEHX₁₇X₁₈X₁₉ | 2180 |
| gagagggca | GCA gag | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QSGDLRRHX₁₇X₁₈ | |
| ctg | AGG GCA | | QSGDLRRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDHLTNHX₁₇ | |
| | CTG | | GKSFSRSDHLTNHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDNLVRH | |
| | | | KCPECGKSFSRSDNLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QSGDLR | |
| | | | GEKPYKCPECGKSFSQSGDLRRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RKDN | |
| | | | QRTHTGEKPYKCPECGKSFSRKD | | LKNHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | NLKNHQRTHTGEKPYKCPECGKS | | DHLTNHX₁₇X₁₈X₁₉HX₂₀X₂₁X₂₂X₂₃X₂₄X₂₅X₂₆X₂₇X₂₈X₂₉X₃₀ | |
| | | | FSRSDHLTNHQRTHTGEKPTGKK | | | |
| | | | TS | | | |
| tagggcagg | TAG GGC | 1083 | LEPGEKPYKCPECGKSFSSRRTC | 1632 | X₁X₂X₃X₄X₅X₆X₇X₈X₉CX₁₀X₁₁CX₁₂X₁₃X₁₄X₁₅X₁₆SRRTCRAHX₁₇X₁₈X₁₉ | 2181 |
| tggccgcgg | AGG TGG | | RAHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDKLTEHX₁₇X₁₈ | |
| cgt | CCG CGG | | RSDKLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RNDTLTEHX₁₇ | |
| | CGT | | GKSFSRNDTLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDHLTTH | |
| | | | KCPECGKSFSRSDHLTTHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDHLT | |
| | | | GEKPYKCPECGKSFSRSDHLTNH | | NHX₁₇X₁₈X₁₉HX₂₀X₂₁X₂₂X₂₃X₂₄X₈X₉CX₁₀X₁₁CX₁₂X₁₃X₁₄X₁₅X₁₆DPGH | |
| | | | QRTHTGEKPYKCPECGKSFSDPG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RE | |
| | | | HLVRHQRTHTGEKPYKCPECGKS | | DNLHTHX₁₇X₁₈X₁₉HX₂₀X₂₁X₂₂X₂₃X₂₄X₂₅X₂₆X₂₇X₂₈X₂₉X₃₀ | |
| | | | FSREDNLHTHQRTHTGEKPTGKK | | | |
| | | | TS | | | |
| gggtagggc | GGG TAG | 1084 | LEPGEKPYKCPECGKSFSRSDKL | 1633 | X₁X₂X₃X₄X₅X₆X₇X₈X₉CX₁₀X₁₁CX₁₂X₁₃X₁₄X₁₅X₁₆RSDKLTEHX₁₇X₁₈X₁₉ | 2182 |
| aggtggccg | GGC AGG | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RNDTLTEHX₁₇X₁₈ | |
| cgg | TGG CCG | | RNDTLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDHLTTHX₁₇ | |
| | CGG | | GKSFSRSDHLTTHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDHLTNH | |
| | | | KCPECGKSFSRSDHLTNHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DPGHLV | |
| | | | GEKPYKCPECGKSFSDPGHLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆REDN | |
| | | | QRTHTGEKPYKCPECGKSFSRED | | LHTHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | NLHTHQRTHTGEKPYKCPECGKS | | DKLVRHX₁₇X₁₈X9HX₂₀X₂₁X₂₂X₂₃X₂₄X₂₅X₂₆X₂₇X₂₈X₂₉X₃₀ | |
| | | | FSRSDKLVRHQRTHTGEKPTGKK | | | |
| | | | TS | | | |
| ccagggtag | CCA GGG | 1085 | LEPGEKPYKCPECGKSFSRNDTL | 1634 | X₁X₂X₃X₄X₅X₆X₇X₈X₉CX₁₀X₁₁CX₁₂X₁₃X₁₄X₁₅X₁₆RNDTLTEHX₁₇X₁₈X₁₉ | 2183 |
| ggcaggtgg | TAG GGC | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDHLTTHX₁₇X₁₈ | |
| ccg | AGG TGG | | RSDHLTTHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDHLTNHX₁₇ | |
| | CCG | | GKSFSRSDHLTNHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DPGHLVRH | |
| | | | KCPECGKSFSDPGHLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆REDNLH | |
| | | | GEKPYKCPECGKSFSREDNLHTH | | THX₁₇X₁₈X₁₉HX₂₀X₂₁X₂₂X₂₃X₂₄X₈X₉CX₁₀X₁₁CX₁₂X₁₃X₁₄X₁₅X₁₆RSDK | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | KLVRHQRTHTGEKPYKCPECGKS | | HSLTEHX₁₇X₁₈X₁₉HX₂₀X₂₁X₂₂X₂₃X₂₄X₂₅X₂₆X₂₇X₂₈X₂₉X₃₀ | |
| | | | FSTSHSLTEHQRTHTGEKPTGKK | | | |
| | | | TS | | | |
| gcggagggc | GCG GAG | 1086 | LEPGEKPYKCPECGKSFSRSDKL | 1635 | X₁X₂X₃X₄X₅X₆X₇X₈X₉CX₁₀X₁₁CX₁₂X₁₃X₁₄X₁₅X₁₆RSDKLTEHX₁₇X₁₈X₁₉ | 2184 |
| gggggcctt | GGC GGG | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TTGALTEHX₁₇X₁₈ | |
| cgg | GGC CTT | | TTGALTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DPGHLVRHX₁₇ | |
| | CGG | | GKSFSDPGHLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDKLVRH | |
| | | | KCPECGKSFSRSDKLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DPGHLV | |
| | | | GEKPYKCPECGKSFSDPGHLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDN | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | NLVRHQRTHTGEKPYKCPECGKS | | DDLVRHX₁₇X₁₈X₁₉HX₂₀X₂₁X₂₂X₂₃X₂₄X₂₅X₂₆X₂₇X₂₈X₂₉X₃₀ | |
| | | | FSRSDDLVRHQRTHTGEKPTGKK | | | |
| | | | TS | | | |
| gaaaggaag | GAA AGG | 1087 | LEPGEKPYKCPECGKSFSQSGDL | 1636 | X₁X₂X₃X₄X₅X₆X₇X₈X₉CX₁₀X₁₁CX₁₂X₁₃X₁₄X₁₅X₁₆QSGDLRRHX₁₇X₁₈X₁₉ | 2185 |
| gcagagagg | AAG GCA | | RRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDHLTNHX₁₇X₁₈ | |
| gca | gag AGG | | RSDHLTNHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDNLVRHX₁₇ | |
| | GCA | | GKSFSRSDNLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QSGDLRRH | |
| | | | KCPECGKSFSQSGDLRRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RKDNLK | |
| | | | GEKPYKCPECGKSFSRKDNLKNH | | NHX₁₇X₁₈X₁₉HX₂₀X₂₁X₂₂X₂₃X₂₄X₈X₉CX₁₀X₁₁CX₁₂X₁₃X₁₄X₁₅X₁₆RSDH | |
| | | | QRTHTGEKPYKCPECGKSFSRSD | | LTNHX₁₇X₁₈X₁₉HX₂₀X₂₁X₂₂X₂₃X₂₄X₈X₉CX₁₀X₁₁CX₁₂X₁₃X₁₄X₁₅X₁₆QS | |
| | | | HLTNHQRTHTGEKPYKCPECGKS | | SNLVRHX₁₇X₁₈X₁₉HX₂₀X₂₁X₂₂X₂₃X₂₄X₂₅X₂₆X₂₇X₂₈X₂₉X₃₀ | |
| | | | FSQSSNLVRHQRTHTGEKPTGKK | | | |
| | | | TS | | | |
| ggcagtggg | GGC AGT | 1088 | LEPGEKPYKCPECGKSFSDPGHL | 1637 | X₁X₂X₃X₄X₅X₆X₇X₈X₉CX₁₀X₁₁CX₁₂X₁₃X₁₄X₁₅X₁₆DPGHLVRHX₁₇X₁₈X₁₉ | 2186 |
| agggcggag | ggg AGG | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDNLVRHX₁₇X₁₈ | |
| ggc | GCG GAG | | RSDNLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDDLVRHX₁₇ | |
| | GGC | | GKSFSRSDDLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDHLTNH | |
| | | | KCPECGKSSRSDHLVTNHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDKLV | |
| | | | GEKPYKCPECGKSFSRSDKLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆HRTT | |
| | | | QRTHTGEKPYKCPECGKSFSHRT | | LTNHX₁₇X₁₈X₁₉HX₂₀X₂₁X₂₂X₂₃X₂₄X₈X₉CX₁₀X₁₁CX₁₂X₁₃X₁₄X₁₅X₁₆DP | |
| | | | TLTNHQRTHTGEKPYKCPECGKS | | GHLVRHX₁₇X₁₈X₁₉HX₂₀X₂₁X₂₂X₂₃X₂₄X₂₅X₂₆X₂₇X₂₈X₂₉X₃₀ | |
| | | | FSDPGHLVRHQRTHTGEKPTGKK | | | |
| | | | TS | | | |
| agtgggagg | AGT ggg | 1089 | LEPGEKPYKCPECGKSFSRSDKL | 1638 | X₁X₂X₃X₄X₅X₆X₇X₈X₉CX₁₀X₁₁CX₁₂X₁₃X₁₄X₁₅X₁₆RSDKLVRHX₁₇X₁₈X₁₉ | 2187 |
| gcggagggc | AGG GCG | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆DPGHLVRHX₁₇X₁₈ | |
| ggg | GAG GGC | | DPGHLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDNLVRHX₁₇ | |
| | GGG | | GKSFSRSDNLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDDLVRH | |
| | | | KCPECGKSFSRSDDLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDHLT | |
| | | | GEKPYKCPECGKSFSRSDHLTNH | | NHX₁₇X₁₈X₁₉HX₂₀X₂₁X₂₂X₂₃X₂₄X₈X₉CX₁₀X₁₁CX₁₂X₁₃X₁₄X₁₅X₁₆RSDK | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆HR | |
| | | | KLVRHQRTHTGEKPYKCPECGKS | | TTLTNHX₁₇X₁₈X₁₉HX₂₀X₂₁X₂₂X₂₃X₂₄X₂₅X₂₆X₂₇X₂₈X₂₉X₃₀ | |
| | | | FSHRTTLTNHQRTHTGEKPTGKK | | | |
| | | | TS | | | |
| agggcggag | AGG GCG | 1090 | LEPGEKPYKCPECGKSFSTTGAL | 1639 | X₁X₂X₃X₄X₅X₆X₇X₈X₉CX₁₀X₁₁CX₁₂X₁₃X₁₄X₁₅X₁₆TTGALTEHX₁₇X₁₈X₁₉ | 2188 |
| ggcgggggc | GAG GGC | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆DPGHLVRHX₁₇X₁₈ | |
| ctt | GGG GGC | | DPGHLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDKLVRHX₁₇ | |
| | CTT | | GKSFSRSDKLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DPGHLVRH | |
| | | | KCPECGKSFSDPGHLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDNLV | |
| | | | GEKYKCPEVCGKSFSRSDNLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDD | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | DLVRHQRTHTGEKPYKCPECGKS | | DHLTNHX₁₇X₁₈X₁₉HX₂₀X₂₁X₂₂X₂₃X₂₄X₂₅X₂₆X₂₇X₂₈X₂₉X₃₀ | |
| | | | FSRSDHLTNHQRTHTGEKPTGKK | | | |
| | | | TS | | | |
| aaggcagag | AAG GCA | 1091 | LEPGEKPYKCPECGKSFSQRAHL | 1640 | X₁X₂X₃X₄X₅X₆X₇X₈X₉CX₁₀X₁₁CX₁₂X₁₃X₁₄X₁₅X₁₆QRAHLERHX₁₇X₁₈X₁₉ | 2189 |
| agggcactg | gag AGG | | ERHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RNDALTEHX₁₇X₁₈ | |
| gga | GCA CTG | | RNDALTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QSGDLRRHX₁₇ | |
| | GGA | | GKSFSQSGDLRRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDHLTNH | |
| | | | KCPECGKSFSRSDHLTNHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDNLV | |
| | | | GEKPYKCPECGKSFSRSDNLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆QSGD | |
| | | | QRTHTGEKPYKCPECGKSFSQSG | | LRRHX₁₇X₁₈X₁₉HX₂₀X₂₁X₂₂X₂₃X₂₄X₈X₉CX₁₀X₁₁CX₁₂X₁₃X₁₄X₁₅X₁₆RK | |
| | | | DLRRHQRTHTGEKPYKCPECGKS | | DNLKNHX₁₇X₁₈X₁₉HX₂₀X₂₁X₂₂X₂₃X₂₄X₂₅X₂₆X₂₇X₂₈X₂₉X₃₀ | |
| | | | FSRKDNLKNHQRTHTGEKPTGKK | | | |
| | | | TS | | | |
| ccgcggcgt | CCG CGG | 102 | LEPGEKPYKCPECGKSFSRSDNL | 103 | X₁X₂X₃X₄X₅X₆X₇X₈X₉CX₁₀X₁₁CX₁₂X₁₃X₁₄X₁₅X₁₆RSDNLVRHX₁₇X₁₈X₁₉ | 313 |
| ggaggcagg | CGT GGA | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDHLTNHX₁₇X₁₈ | |
| gag | GGC AGG | | RSDHLTNHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DPGHLVRHX₁₇ | |
| | GAG | | GKSFSDPGHLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QRAHLERH | |
| | | | KCPECGKSFSQRAHLERHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆SRRTCR | |
| | | | GEKPYKCPECGKSFSSRRTCRAH | | AHX₁₇X₁₈X₁₉HX₂₀X₂₁X₂₂X₂₃X₂₄X₈X₉CX₁₀X₁₁CX₁₂X₁₃X₁₄X₁₅X₁₆RSDK | |
| | | | QRTHTGEKPYKCPECGKSFSRSD | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆RN | |
| | | | KLTEHQRTHTGEKPYKCPECGKS | | DTLTEHX₁₇X₁₈X₁₉HX₂₀X₂₁X₂₂X₂₃X₂₄X₂₅X₂₆X₂₇X₂₈X₂₉X₃₀ | |
| | | | FSRNDTLTEHQRTHTGEKPTGKK | | | |
| | | | TS | | | |
| cgtggaggc | CGT GGA | 1092 | LEPGEKPYKCPECGKSFSRSDDL | 1641 | X₁X₂X₃X₄X₅X₆X₇X₈X₉CX₁₀X₁₁CX₁₂X₁₃X₁₄X₁₅X₁₆RSDDLVRHX₁₇X₁₈X₁₉ | 2190 |
| agggagaat | GGC AGG | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TTGNLTVHX₁₇X₁₈ | |
| gcg | gag AAT | | TTGNLTVHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDNLVRHX₁₇ | |
| | GCG | | GKSFSRSDNLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDHLTNH | |
| | | | KCPECGKSFSRSDHLTNHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DPGHLV | |
| | | | GEKPYKCPECGKSFSDPGHLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆QRAH | |
| | | | QRTHTGEKPYKCPECGKSFSQRA | | LERHX₁₇X₁₈X₁₉HX₂₀X₂₁X₂₂X₂₃X₂₄X₈X₉CX₁₀X₁₁CX₁₂X₁₃X₁₄X₁₅X₁₆SR | |
| | | | HLERHQRTHTGEKPYKCPECGKS | | RTCRAHX₁₇X₁₈X₁₉HX₂₀X₂₁X₂₂X₂₃X₂₄X₂₅X₂₆X₂₇X₂₈X₂₉X₃₀ | |
| | | | FSSRRTCRAHQRTHTGEKPTGKK | | | |
| | | | TS | | | |
| gcagagagg | GCA gag | 1093 | LEPGEKPYKCPECGKSFSQRAHL | 1642 | X₁X₂X₃X₄X₅X₆X₇X₈X₉CX₁₀X₁₁CX₁₂X₁₃X₁₄X₁₅X₁₆QRAHLERHX₁₇X₁₈X₁₉ | 2191 |
| gcactggga | AGG GCA | | ERHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QRAHLERHX₁₇X₁₈ | |
| gga | CTG GGA | | QRAHLERHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RNDALTEHX₁₇ | |
| | GGA | | GKSFSRNDALTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QSGDLRRH | |
| | | | KCPECGKSFSQSGDLRRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDHLT | |
| | | | GEKPYKCPECGKSFSRSDHLTNH | | NHX₁₇X₁₈X₁₉HX₂₀X₂₁X₂₂X₂₃X₂₄X₈X₉CX₁₀X₁₁CX₁₂X₁₃X₁₄X₁₅X₁₆RSDN | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆QS | |
| | | | NLVRHQRTHTGEKPYKCPECGKS | | GDLRRHX₁₇X₁₈X₁₉HX₂₀X₂₁X₂₂X₂₃X₂₄X₂₅X₂₆X₂₇X₂₈X₂₉X₃₀ | |
| | | | FSQSGDLRRHQRTHTGEKPTGKK | | | |
| | | | TS | | | |
| gagagggca | gag AGG | 1094 | LEPGEKPYKCPECGKSFSDPGHL | 1643 | X₁X₂X₃X₄X₅X₆X₇X₈X₉CX₁₀X₁₁CX₁₂X₁₃X₁₄X₁₅X₁₆DPGHLVRHX₁₇X₁₈X₁₉ | 2192 |
| ctgggagga | GCA CTG | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QRAHLERHX₁₇X₁₈ | |
| ggc | GGA GGA | | QRAHLERHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QRAHLERHX₁₇ | |
| | GGC | | GKSFSQRAHLERHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RNDALTEH | |
| | | | KCPECGKSFSRNDALTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QSGDLR | |
| | | | GEKPYKCPECGKSFSQSGDLRRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDH | |
| | | | QRTHTGEKPYKCPECGKSFSRSD | | LTNHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | HLTNHQRTHTGEKPYKCPECGKS | | DNLVRHX₁₇X₁₈X₁₉HX₂₀X₂₁X₂₂X₂₃X₂₄X₂₅X₂₆X₂₇X₂₈X₂₉X₃₀ | |
| | | | FSRSDNLVRHQRTHTGEKPTGKK | | | |
| | | | TS | | | |
| aggtggccg | AGG TGG | 1095 | LEPGEKPYKCPECGKSFSDPGHL | 1644 | X₁X₂X₃X₄X₅X₆X₇X₈X₉CX₁₀X₁₁CX₁₂X₁₃X₁₄X₁₅X₁₆DPGHLVRHX₁₇X₁₈X₁₉ | 2193 |
| cggcgtgga | CCG CGG | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QRAHLERHX₁₇X₁₈ | |
| ggc | CGT GGA | | QRAHLERHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆SRRTCRAHX₁₇ | |
| | GGC | | GKSFSSRRTCRAHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDKLTEH | |
| | | | KCPECGKSFSRSDKLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RNDTLT | |
| | | | GEKPYKCPECGKSFSRNDTLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆RSDH | |
| | | | QRTHTGEKPYKCPECGKSFSRSD | | LTTHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | HLTTHQRTHTGEKPYKCPECGKS | | DHLTNHX₁₇X₁₈X₁₉HX₂₀X₂₁X₂₂X₂₃X₂₄X₂₅X₂₆X₂₇X₂₈X₂₉X₃₀ | |
| | | | FSRSDHLTNHQRTHTGEKPTGKK | | | |
| | | | TS | | | |
| tggccgcgg | TGG CCG | 1096 | LEPGEKPYKCPECGKSFSRSDHL | 1645 | X₁X₂X₃X₄X₅X₆X₇X₈X₉CX₁₀X₁₁CX₁₂X₁₃X₁₄X₁₅X₁₆RSDHLTNHX₁₇X₁₈X₁₉ | 2194 |
| cgtggaggc | CGG CGT | | TNHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆DPGHLVRHX₁₇X₁₈ | |
| agg | GGA GGC | | DPGHLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QRAHLERHX₁₇ | |
| | AGG | | GKSFSQRAHLERHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆SRRTCRAH | |
| | | | KCPECGKSFSSRRTCRAHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDKLT | |
| | | | GEKPYKCPECGKSFSRSDKLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆RNDT | |
| | | | QRTHTGEKPYKCPECGKSFSRND | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | TLTEHQRTHTGEKPYKCPECGKS | | DHLTTHX₁₇X₁₈X₁₉HX₂₀X₂₁X₂₂X₂₃X₂₄X₂₅X₂₆X₂₇X₂₈X₂₉X₃₀ | |
| | | | FSRSDHLTTHQRTHTGEKPTGKK | | | |
| | | | TS | | | |
| ggcaggtgg | GGC AGG | 1097 | LEPGEKPYKCPECGKSFSQRAHL | 1646 | X₁X₂X₃X₄X₅X₆X₇X₈X₉CX₁₀X₁₁CX₁₂X₁₃X₁₄X₁₅X₁₆QRAHLERHX₁₇X₁₈X₁₉ | 2195 |
| ccgcggcgt | TGG CCG | | ERHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆SRRTCRAHX₁₇X₁₈ | |
| gga | CGG CGT | | SRRTCRAHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDKLTEHX₁₇ | |
| | GGA | | GKSFSRSDKLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RNDTLTEH | |
| | | | KCPECGKSFSRNDTLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDHLT | |
| | | | GEKPYKCPECGKSFSRSDHLTTH | | THX₁₇X₁₈X₁₉HX₂₀X₂₁X₂₂X₂₃X₂₄X₈X₉CX₁₀X₁₁CX₁₂X₁₃X₁₄X₁₅X₁₆RSDH | |
| | | | QRTHTGEKPYKCPECGKSFSRSD | | LTNHX₁₇X₁₈X₁₉HX₂₀X₂₁X₂₂X₂₃X₂₄X₈X₉CX₁₀X₁₁CX₁₂X₁₃X₁₄X₁₅X₁₆DP | |
| | | | HLTNHQRTHTGEKPYKCPECGKS | | GHLVRHX₁₇X₁₈X₁₉HX₂₀X₂₁X₂₂X₂₃X₂₄X₂₅X₂₆X₂₇X₂₈X₂₉X₃₀ | |
| | | | FSDPGHLVRHQRTHTGEKPTGKK | | | |
| | | | TS | | | |
| ggaggcagg | GGA GGC | 1098 | LEPGEKPYKCPECGKSFSTHLDL | 1647 | X₁X₂X₃X₄X₅X₆X₇X₈X₉CX₁₀X₁₁CX₁₂X₁₃X₁₄X₁₅X₁₆THLDLIRHX₁₇X₁₈X₁₉ | 2196 |
| gagaatgcg | AGG gag | | IRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDDLVRHX₁₇X₁₈ | |
| act | AAT gcg | | RSDDLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TTGNLTVHX₁₇ | |
| | ACT | | GKSFSTTGNLTVHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDNLVRH | |
| | | | KCPECGKSFSRSDNLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDHLT | |
| | | | GEKPYKCPECGKSFSRSDHLTNH | | NHX₁₇X₁₈X₁₉HX₂₀X₂₁X₂₂X₂₃X₂₄X₈X₉CX₁₀X₁₁CX₁₂X₁₃X₁₄X₁₅X₁₆DPGH | |
| | | | QRTHTGEKPYKCPECGKSFSDPG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆QR | |
| | | | HLVRHQRTHTGEKPYKCPECGKS | | AHLERHX₁₇X₁₃X₁₉HX₂₀X₂₁X₂₂X₂₃X₂₄X₂₅X₂₆X₂₇X₂₈X₂₉X₃₀ | |
| | | | FSQRAHLERHQRTHTGEKPTGKK | | | |
| | | | TS | | | |
| gcactggga | GCA CTG | 1099 | LEPGEKPYKCPECGKSFSRSDKL | 1648 | X₁X₂X₃X₄X₅X₆X₇X₈X₉CX₁₀X₁₁CX₁₂X₁₃X₁₄X₁₅X₁₆RSDKLVRHX₁₇X₁₈X₁₉ | 2197 |
| ggaggcagt | GGA GGA | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆HRTTLTNHX₁₇X₁₈ | |
| ggg | GGC AGT | | HRTTLTNHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DPGHLVRHX₁₇ | |
| | GGG | | GKSFSDPGHLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QRAHLERH | |
| | | | KCPECGKSFSQRAHLERHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QRAHLE | |
| | | | GEKPYKCPECGKSFSQRAHLERH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RNDA | |
| | | | QRTHTGEKPYKCPECGKSFSRND | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆QS | |
| | | | ALTEHQRTHTGEKPYKCPECGKS | | GDLRRHX₁₇X₁₈X₁₉HX₂₀X₂₁X₂₂X₂₃X₂₄X₂₅X₂₆X₂₇X₂₈X₂₉X₃₀ | |
| | | | FSQSGDLRRHQRTHTGEKPTGKK | | | |
| | | | TS | | | |
| ggaggcagt | GGA GGC | 1100 | LEPGEKPYKCPECGKSFSRSDNL | 1649 | X₁X₂X₃X₄X₅X₆X₇X₈X₉CX₁₀X₁₁CX₁₂X₁₃X₁₄X₁₅X₁₆RSDNLVRHX₁₇X₁₈X₁₉ | 2198 |
| gggagggcg | AGT ggg | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDDLVRHX₁₇X₁₈ | |
| gag | AGG GCG | | RSDDLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDHLTNHX₁₇ | |
| | GAG | | GKSFSRSDHLTNHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDKLVRH | |
| | | | KCPECGKSFSRSDKLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆HRTTLT | |
| | | | GEKPYKCPECGKSFSHRTTLTNH | | NHX₁₇X₁₈X₁₉HX₂₀X₂₁X₂₂X₂₃X₂₄X₈X₉CX₁₀X₁₁CX₁₂X₁₃X₁₄X₁₅X₁₆DPGH | |
| | | | QRTHTGEKPYKCPECGKSFSDPG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆QR | |
| | | | HLVRHQRTHTGEKPYKCPECGKS | | AHLERHX₁₇X₁₈X₁₉HX₂₀X₂₁X₂₂X₂₃X₂₄X₂₅X₂₆X₂₇X₂₈X₂₉X₃₀ | |
| | | | FSQRAHLERHQRTHTGEKPTGKK | | | |
| | | | TS | | | |
| agggcactg | AGG GCA | 1101 | LEPGEKPYKCPECGKSFSHRTTL | 1650 | X₁X₂X₃X₄X₅X₆X₇X₈X₉CX₁₀X₁₁CX₁₂X₁₃X₁₄X₁₅X₁₆HRTTLTNHX₁₇X₁₈X₁₉ | 2199 |
| ggaggaggc | CTG GGA | | TNHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆DPGHLVRHX₁₇X₁₈ | |
| agt | GGA GGC | | DPGHLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QRAHLERHX₁₇ | |
| | AGT | | GKSFSQRAHLERHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QRAHLERH | |
| | | | KCPECGKSFSQRAHLERHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RNDALT | |
| | | | GEKPYKCPECGKSFSRNDALTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆QSGD | |
| | | | QRTHTGEKPYKCPECGKSFSQSG | | LRRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | DLRRHQRTHTGEKPYKCPECGKS | | DHLTNHX₁₇X₁₈X₁₉HX₂₀X₂₁X₂₂X₂₃X₂₄X₂₅X₂₆X₂₇X₂₈X₂₉X₃₀ | |
| | | | FSRSDHLTNHQRTHTGEKPTGKK | | | |
| | | | TS | | | |
| ctgggagga | CTG GGA | 1102 | LEPGEKPYKCPECGKSFSRSDHL | 1651 | X₁X₂X₃X₄X₅X₆X₇X₈X₉CX₁₀X₁₁CX₁₂X₁₃X₁₄X₁₅X₁₆RSDHLTNHX₁₇X₁₈X₁₉ | 2200 |
| ggcagtggg | GGA GGC | | TNHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDKLVRHX₁₇X₁₈ | |
| agg | AGT ggg | | RSDKLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆HRTTLTNHX₁₇ | |
| | AGG | | GKSFSHRTTLTNHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DPGHLVRH | |
| | | | KCPECGKSFSDPGHLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QRAHLE | |
| | | | GEKPYKCPECGKSFSQRAHLERH | | RHX₁₇X₁₈X₁₉HX₂₀X₂₁X₂₂X₂₃X₂₄X₈X₉CX₁₀X₁₁CX₁₂X₁₃X₁₄X₁₅X₁₆QRAH | |
| | | | QRTHTGEKPYKCPECGKSFSQRA | | LERHX₁₇X₁₈X₁₉HX₂₀X₂₁X₂₂X₂₃X₂₄X₈X₉CX₁₀X₁₁CX₁₂X₁₃X₁₄X₁₅X₁₆RN | |
| | | | HLERHQRTHTGEKPYKCPECGKS | | DALTEHX₁₇X₁₈X₁₉HX₂₀X₂₁X₂₂X₂₃X₂₄X₂₅X₂₆X₂₇X₂₈X₂₉X₃₀ | |
| | | | FSRNDALTEHQRTHTGEKPTGKK | | | |
| | | | TS | | | |
| ggtgggcgc | GGT ggg | 1103 | LEPGEKPYKCPECGKSFSDPGHL | 1652 | X₁X₂X₃X₄X₅X₆X₇X₈X₉CX₁₀X₁₁CX₁₂X₁₃X₁₄X₁₅X₁₆DPGHLVRHX₁₇X₁₈X₁₉ | 2201 |
| ccagggtag | CGC CCA | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆REDNLHTHX₁₇X₁₈ | |
| ggc | GGG TAG | | REDNLHTHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDKLVRHX₁₇ | |
| | GGC | | GKSFSRSDKLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSHSLTEH | |
| | | | KCPECGKSFSTSHSLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆HTGHLL | |
| | | | GEKPYKCPECGKSFSHTGHLLEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆RSDK | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | KLVRHQRTHTGEKPYKCPECGKS | | GHLVRHX₁₇X₁₈X₁₉HX₂₀X₂₁X₂₂X₂₃X₂₄X₂₅X₂₆X₂₇X₂₈X₂₉X₃₀ | |
| | | | FSTSGHLVRHQRTHTGEKPTGKK | | | |
| | | | TS | | | |
| cggggtggg | CGG GGT | 1104 | LEPGEKPYKCPECGKSFSREDNL | 1653 | X₁X₂X₃X₄X₅X₆X₇X₈X₉CX₁₀X₁₁CX₁₂X₁₃X₁₄X₁₅X₁₆REDNLHTHX₁₇X₁₈X₁₉ | 2202 |
| cgcccaggg | ggg CGC | | HTHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDKLVRHX₁₇X₁₈ | |
| tag | CCA GGG | | RSDKLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSHSLTEHX₁₇ | |
| | TAG | | GKSFSTSHSLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆HTGHLLEH | |
| | | | KCPECGKSFSHTGHLLEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDKLV | |
| | | | GEKPYKCPECGKSFSRSDKLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆TSGH | |
| | | | QRTHTGEKPYKCPECGKSFSTSG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | HLVRHQRTHTGEKPYKCPECGKS | | DKLTEHX₁₇X₁₈X₁₉HX₂₀X₂₁X₂₂X₂₃X₂₄X₂₅X₂₆X₂₇X₂₈X₂₉X₃₀ | |
| | | | FSRSDKLTEHQRTHTGEKPTGKK | | | |
| | | | TS | | | |
| ggcgggggc | GGC GGG | 1105 | LEPGEKPYKCPECGKSFSRSDKL | 1654 | X₁X₂X₃X₄X₅X₆X₇X₈X₉CX₁₀X₁₁CX₁₂X₁₃X₁₄X₁₅X₁₆RSDKLVRHX₁₇X₁₈X₁₉ | 2203 |
| cttcggggt | GGC CTT | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSGHLVRHX₁₇X₁₈ | |
| ggg | CGG GGT | | TSGHLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDKLTEHX₁₇ | |
| | GGG | | GKSFSRSDKLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TTGALTEH | |
| | | | KCPECGKSFSTTGALTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DPGHLV | |
| | | | GEKPYKCPECGKSFSDPGHLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDK | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆DP | |
| | | | KLVRHQRTHTGEKPYKCPECGKS | | GHLVRHX₁₇X₁₈X₁₉HX₂₀X₂₁X₂₂X₂₃X₂₄X₂₅X₂₆X₂₇X₂₈X₂₉X₃₀ | |
| | | | FSDPGHLVRHQRTHTGEKPTGKK | | | |
| | | | TS | | | |
| gagggcggg | GAG GGC | 1106 | LEPGEKPYKCPECGKSFSTSGHL | 1655 | X₁X₂X₃X₄X₅X₆X₇X₈X₉CX₁₀X₁₁CX₁₂X₁₃X₁₄X₁₅X₁₆TSGHLVRHX₁₇X₁₈X₁₉ | 2204 |
| ggccttcgg | GGG GGC | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDKLTEHX₁₇X₁₈ | |
| ggt | CTT CGG | | RSDKLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TTGALTEHX₁₇ | |
| | GGT | | GKSFSTTGALTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DPGHLVRH | |
| | | | KCPECGKSFSDPGHLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDKLV | |
| | | | GEKPYKCPECGKSFSRSDKLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆DPGH | |
| | | | QRTHTGEKPYKCPECGKSFSDPG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | HLVRHQRTHTGEKPYKCPECGKS | | DNLVRHX₁₇X₁₈X₁₉HX₂₀X₂₁X₂₂X₂₃X₂₄X₂₅X₂₆X₂₇X₂₈X₂₉X₃₀ | |
| | | | FSRSDNLVRHQRTHTGEKPTGKK | | | |
| | | | TS | | | |
| gggggcctt | GGG GGC | 1107 | LEPGEKPYKCPECGKSFSHTGHL | 1656 | X₁X₂X₃X₄X₅X₆X₇X₈X₉CX₁₀X₁₁CX₁₂X₁₃X₁₄X₁₅X₁₆HTGHLLEHX₁₇X₁₈X₁₉ | 2205 |
| cggggtggg | CTT CGG | | LEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDKLVRHX₁₇X₁₈ | |
| cgc | GGT ggg | | RSDKLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSGHLVRHX₁₇ | |
| | CGC | | GKSFSTSGHLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDKLTEH | |
| | | | KCPECGKSFSRSDKLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TTGALT | |
| | | | GEKPYKCPECGKSFSTTGALTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆DPGH | |
| | | | QRTHTGEKPYKCPECGKSFSDPG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | HLVRHQRTHTGEKPYKCPECGKS | | DKLVRHX₁₇X₁₈X₁₉HX₂₀X₂₁X₂₂X₂₃X₂₄X₂₅X₂₆X₂₇X₂₈X₂₉X₃₀ | |
| | | | FSRSDKLVRHQRTHTGEKPTGKK | | | |
| | | | TS | | | |
| ggaggaggc | GGA GGA | 1108 | LEPGEKPYKCPECGKSFSRSDDL | 1657 | X₁X₂X₃X₄X₅X₆X₇X₈X₉CX₁₀X₁₁CX₁₂X₁₃X₁₄X₁₅X₁₆RSDDLVRHX₁₇X₁₈X₁₉ | 2206 |
| agtgggagg | GGC AGT | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDHLTNHX₁₇X₁₈ | |
| gcg | ggg AGG | | RSDHLTNHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDKLVRHX₁₇ | |
| | GCG | | GKSFSRSDKLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆HRTTLTNH | |
| | | | KCPECGKSFSHRTTLTNHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DPGHLV | |
| | | | GEKPYKCPECGKSFSDPGHLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆QRAH | |
| | | | QRTHTGEKPYKCPECGKSFSQRA | | LERHX₁₇X₁₈X₁₉HX₂₀X₂₁X₂₂X₂₃X₂₄X₈X₉CX₁₀X₁₁CX₁₂X₁₃X₁₄X₁₅X₁₆QR | |
| | | | HLERHQRTHTGEKPYKCPECGKS | | AHLERHX₁₇X₁₈X₁₉HX₂₀X₂₁X₂₂X₂₃X₂₄X₂₅X₂₆X₂₇X₂₈X₂₉X₃₀ | |
| | | | FSQRAHLERHQRTHTGEKPTGKK | | | |
| | | | TS | | | |
| ggccttcgg | GGC CTT | 1109 | LEPGEKPYKCPECGKSFSTSHSL | 1658 | X₁X₂X₃X₄X₅X₆X₇X₈X₉CX₁₀X₁₁CX₁₂X₁₃X₁₄X₁₅X₁₆TSHSLTEHX₁₇X₁₈X₁₉ | 2207 |
| ggtgggcgc | CGG GGT | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆HTGHLLEHX₁₇X₁₈ | |
| cca | ggg CGC | | HTGHLLEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDKLVRHX₁₇ | |
| | CCA | | GKSFSRSDKLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSGHLVRH | |
| | | | KCPECGKSFSTSGHLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDKLT | |
| | | | GEKPYKCPECGKSFSRSDKLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆TTGA | |
| | | | QRTHTGEKPYKCPECGKSFSTTG | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆DP | |
| | | | ALTEHQRTHTGEKPYKCPECGKS | | GHLVRHX₁₇X₁₈X₁₉HX₂₀X₂₁X₂₂X₂₃X₂₄X₂₅X₂₆X₂₇X₂₈X₂₉X₃₀ | |
| | | | FSDPGHLVRHQRTHTGEKPTGKK | | | |
| | | | TS | | | |
| cttcggggt | CTT CGG | 1110 | LEPGEKPYKCPECGKSFSRSDKL | 1659 | X₁X₂X₃X₄X₅X₆X₇X₈X₉CX₁₀X₁₁CX₁₂X₁₃X₁₄X₁₅X₁₆RSDKLVRHX₁₇X₁₈X₁₉ | 2208 |
| gggcgccca | GGT ggg | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSHSLTEHX₁₇X₁₈ | |
| ggg | CGC CCA | | TSHSLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆HTGHLLEHX₁₇ | |
| | GGG | | GKSFSHTGHLLEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDKLVRH | |
| | | | KCPECGKSFSRSDKLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSGHLV | |
| | | | GEKPYKCPECGKSFSTSGHLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDK | |
| | | | QRTHTGEKPYKCPECGKSFSRSD | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆TT | |
| | | | KLTEHQRTHTGEKPYKCPECGKS | | GALTEHX₁₇X₁₈X₁₉HX₂₀X₂₁X₂₂X₂₃X₂₄X₂₅X₂₆X₂₇X₂₈X₂₉X₃₀ | |
| | | | FSTTGALTEHQRTHTGEKPTGKK | | | |
| | | | TS | | | |
| cagagaggg | CAG AGA | 1111 | LEPGEKPYKCPECGKSFSRSDNL | 1660 | X₁X₂X₃X₄X₅X₆X₇X₈X₉CX₁₀X₁₁CX₁₂X₁₃X₁₄X₁₅X₁₆RSDNLVRHX₁₇X₁₈X₁₉ | 2209 |
| cactgggag | ggg CAC | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDNLVRHX₁₇X₁₈ | |
| gag | TGG GAG | | RSDNLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDHLTTHX₁₇ | |
| | GAG | | GKSFSRSDHLTTHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆SKKALTEH | |
| | | | KCPECGKSFSSKKALTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDKLV | |
| | | | GEKPYKCPECGKSFSRSDKLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆QLAH | |
| | | | QRTHTGEKPYKCPECGKSFSQLA | | LRAHX₁₇X₁₈X₁₉HX₂₀X₂₁X₂₂X₂₃X₂₄X₈X₉CX₁₀X₁₁CX₁₂X₁₃X₁₄X₁₅X₁₆RA | |
| | | | HLRAHQRTHTGEKPYKCPECGKS | | DNLTEHX₁₇X₁₈X₁₉HX₂₀X₂₁X₂₂X₂₃X₂₄X₂₅X₂₆X₂₇X₂₈X₂₉X₃₀ | |
| | | | FSRADNLTEHQRTHTGEKPTGKK | | | |
| | | | TS | | | |
| agagggcac | AGA ggg | 1112 | LEPGEKPYKCPECGKSFSQSGDL | 1661 | X₁X₂X₃X₄X₅X₆X₇X₈X₉CX₁₀X₁₁CX₁₂X₁₃X₁₄X₁₅X₁₆QSGDLRRHX₁₇X₁₈X₁₉ | 2210 |
| tgggaggag | CAC TGG | | RRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDNLVRHX₁₇X₁₈ | |
| gca | GAG GAG | | RSDNLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDNLVRHX₁₇ | |
| | GCA | | GKSFSRSDNLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDHLTTH | |
| | | | KCPECGKSFSRSDHLTTHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆SKKALT | |
| | | | GEKPYKCPECGKSFSSKKALTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆RSDK | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆QL | |
| | | | KLVRHQRTHTGEKPYKCPECGKS | | AHLRAHX₁₇X₁₈X₁₉HX₂₀X₂₁X₂₂X₂₃X₂₄X₂₅X₂₆X₂₇X₂₈X₂₉X₃₀ | |
| | | | FSQLAHLRAHQRTHTGEKPTGKK | | | |
| | | | TS | | | |
| gggcactgg | ggg CAC | 1113 | LEPGEKPYKCPECGKSFSRSDEL | 1662 | X₁X₂X₃X₄X₅X₆X₇X₈X₉CX₁₀X₁₁CX₁₂X₁₃X₁₄X₁₅X₁₆RSDELVRHX₁₇X₁₈X₁₉ | 2211 |
| gaggaggca | TGG GAG | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QSGDLRRHX₁₇X₁₈ | |
| gtg | GAG GCA | | QSGDLRRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDNLVRHX₁₇ | |
| | GTG | | GKSFSRSDNLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDNLVRH | |
| | | | KCPECGKSFSRSDNLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDHLT | |
| | | | GEKPYKCPECGKSFSRSDHLTTH | | THX₁₇X₁₈X₁₉HX₂₀X₂₁X₂₂X₂₃X₂₄X₈X₉CX₁₀X₁₁CX₁₂X₁₃X₁₄X₁₅X₁₆SKKA | |
| | | | QRTHTGEKPYKCPECGKSFSSKK | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | ALTEHQRTHTGEKPYKCPECGKS | | DKLVRHX₁₇X₁₈X₁₉HX₂₀X₂₁X₂₂X₂₃X₂₄X₂₅X₂₆X₂₇X₂₈X₂₉X₃₀ | |
| | | | FSRSDKLVRHQRTHTGEKPTGKK | | | |
| | | | TS | | | |
| aggcagaga | AGG CAG | 1114 | LEPGEKPYKCPECGKSFSRSDNL | 1663 | X₁X₂X₃X₄X₅X₆X₇X₈X₉CX₁₀X₁₁CX₁₂X₁₃X₁₄X₁₅X₁₆RSDNLVRHX₁₇X₁₈X₁₉ | 2212 |
| gggcactgg | AGA ggg | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDHLTTHX₁₇X₁₈ | |
| gag | CAC TGG | | RSDHLTTHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆SKKALTEHX₁₇ | |
| | GAG | | GKSFSSKKALTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDKLVRH | |
| | | | KCPECGKSFSRSDKLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QLAHLR | |
| | | | GEKPYKCPECGKSFSQLAHLRAH | | AHX₁₇X₁₈X₁₉HX₂₀X₂₁X₂₂X₂₃X₂₄X₈X₉CX₁₀X₁₁CX₁₂X₁₃X₁₄X₁₅X₁₆RADN | |
| | | | QRTHTGEKPYKCPECGKSFSRAD | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | NLTEHQRTHTGEKPYKCPECGKS | | DHLTNHX₁₇X₁₈X₁₉HX₂₀X₂₁X₂₂X₂₃X₂₄X₂₅X₂₆X₂₇X₂₈X₂₉X₃₀ | |
| | | | FSRSDHLTNHQRTHTGEKPTGKK | | | |
| | | | TS | | | |
| gaggaggca | GAG GAG | 1115 | LEPGEKPYKCPECGKSFSRSDKL | 1664 | X₁X₂X₃X₄X₅X₆X₇X₈X₉CX₁₀X₁₁CX₁₂X₁₃X₁₄X₁₅X₁₆RSDKLTEHX₁₇X₁₈X₁₉ | 2213 |
| gtgggaggg | GCA GTG | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDKLVRHX₁₇X₁₈ | |
| cgg | GGA ggg | | RSDKLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QRAHLERHX₁₇ | |
| | CGG | | GKSFSQRAHLERHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDELVRH | |
| | | | KCPECGKSFSRSDELVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QSGDLR | |
| | | | GEKPYKCPECGKSFSQSGDLRRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDN | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | NLVRHQRTHTGEKPYKCPECGKS | | DNLVRHX₁₇X₁₈X₁₉HX₂₀X₂₁X₂₂X₂₃X₂₄X₂₅X₂₆X₂₇X₂₈X₂₉X₃₀ | |
| | | | FSRSDNLVRHQRTHTGEKPTGKK | | | |
| | | | TS | | | |
| tgggaggag | TGG GAG | 1116 | LEPGEKPYKCPECGKSFSRSDKL | 1665 | X₁X₂X₃X₄X₅X₆X₇X₈X₉CX₁₀X₁₁CX₁₂X₁₃X₁₄X₁₅X₁₆RSDKLVRHX₁₇X₁₈X₁₉ | 2214 |
| gcagtggga | GAG GCA | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QRAHLERHX₁₇X₁₈ | |
| ggg | GTG GGA | | QRAHLERHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDELVRHX₁₇ | |
| | GGG | | GKSFSRSDELVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QSGDLRRH | |
| | | | KCPECGKSFSQSGDLRRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDNLV | |
| | | | GEKPYKCPECGKSFSRSDNLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDN | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | NLVRHQRTHTGEKPYKCPECGKS | | DHLTTHX₁₇X₁₈X₁₉HX₂₀X₂₁X₂₂X₂₃X₂₄X₂₅X₂₆X₂₇X₂₈X₂₉X₃₀ | |
| | | | FSRSDHLTTHQRTHTGEKPTGKK | | | |
| | | | TS | | | |
| gaggcagtg | GAG GCA | 1117 | LEPGEKPYKCPECGKSFSRSDHL | 1666 | X₁X₂X₃X₄X₅X₆X₇X₈X₉CX₁₀X₁₁CX₁₂X₁₃X₁₄X₁₅X₁₆RSDHLTNHX₁₇X₁₈X₁₉ | 2215 |
| ggagggcgg | GTG GGA | | TNHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDKLTEHX₁₇X₁₈ | |
| agg | ggg CGG | | RSDKLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDKLVRHX₁₇ | |
| | AGG | | GKSFSRSDKLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QRAHLERH | |
| | | | KCPECGKSFSQRAHLERHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDELV | |
| | | | GEKPYKCPECGKSFSRSDELVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆QSGD | |
| | | | QRTHTGEKPYKCPECGKSFSQSG | | LRRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | DLRRHQRTHTGEKPYKCPECGKS | | DNLVRHX₁₇X₁₈X₁₉HX₂₀X₂₁X₂₂X₂₃X₂₄X₂₅X₂₆X₂₇X₂₈X₂₉X₃₀ | |
| | | | FSRSDNLVRHQRTHTGEKPTGKK | | | |
| | | | TS | | | |
| cactgggag | CAC TGG | 1118 | LEPGEKPYKCPECGKSFSQRAHL | 1667 | X₁X₂X₃X₄X₅X₆X₇X₈X₉CX₁₀X₁₁CX₁₂X₁₃X₁₄X₁₅X₁₆QRAHLERHX₁₇X₁₈X₁₉ | 2216 |
| gaggcagtg | GAG GAG | | ERHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDELVRHX₁₇X₁₈ | |
| gga | GCA GTG | | RSDELVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QSGDLRRHX₁₇ | |
| | GGA | | GKSFSQSGDLRRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDNLVRH | |
| | | | KCPECGKSFSRSDNLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDNLV | |
| | | | GEKPYKCPECGKSFSRSDNLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDH | |
| | | | QRTHTGEKPYKCPECGKSFSRSD | | LTTHX₁₇X₁₈X₁₉HX₂₀X₂₁X₂₂X₂₃X₂₄X₈X₉CX₁₀X₁₁CX₁₂X₁₃X₁₄X₁₅X₁₆SK | |
| | | | HLTTHQRTHTGEKPYKCPECGKS | | KALTEHX₁₇X₁₈X₁₉HX₂₀X₂₁X₂₂X₂₃X₂₄X₂₅X₂₆X₂₇X₂₈X₂₉X₃₀ | |
| | | | FSSKKALTEHQRTHTGEKPTGKK | | | |
| | | | TS | | | |
| aaaggaagg | AAA GGA | 1119 | LEPGEKPYKCPECGKSFSSKKAL | 1668 | X₁X₂X₃X₄X₅X₆X₇X₈X₉CX₁₀X₁₁CX₁₂X₁₃X₁₄X₁₅X₁₆SKKALTEHX₁₇X₁₈X₁₉ | 2217 |
| cagagaggg | AGG CAG | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDKLVRHX₁₇X₁₈ | |
| cac | AGA ggg | | RSDKLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QLAHLRAHX₁₇ | |
| | CAC | | GKSFSQLAHLRAHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RADNLTEH | |
| | | | KCPECGKSFSRADNLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDHLT | |
| | | | GEKPYKCPECGKSFSRSDHLTNH | | NHX₁₇X₁₈X₁₉HX₂₀X₂₁X₂₂X₂₃X₂₄X₈X₉CX₁₀X₁₁CX₁₂X₁₃X₁₄X₁₅X₁₆QRAH | |
| | | | QRTHTGEKPYKCPECGKSFSQRA | | LERHX₁₇X₁₈X₁₉HX₂₀X₂₁X₂₂X₂₃X₂₄X₈X₉CX₁₀X₁₁CX₁₂X₁₃X₁₄X₁₅X₁₆QR | |
| | | | HLERHQRTHTGEKPYKCPECGKS | | ANLRAHX₁₇X₁₈X₁₉HX₂₀X₂₁X₂₂X₂₃X₂₄X₂₅X₂₆X₂₇X₂₈X₂₉X₃₀ | |
| | | | FSQRANLRAHQRTHTGEKPTGKK | | | |
| | | | TS | | | |
| ggaaggcag | GGA AGG | 1120 | LEPGEKPYKCPECGKSFSRSDHL | 1669 | X₁X₂X₃X₄X₅X₆X₇X₈X₉CC10X₁₁CX₁₂X₁₃X₁₄X₁₅X₁₆RSDHLTTHX₁₇X₁₈X₁₉ | 2218 |
| agagggcac | CAG AGA | | TTHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆SKKALTEHX₁₇X₁₈ | |
| tgg | ggg CAC | | SKKALTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDKLVRHX₁₇ | |
| | TGG | | GKSFSRSDKLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QLAHLRAH | |
| | | | KCPECGKSFSQLAHLRAHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RADNLT | |
| | | | GEKPYKCPECGKSFSRADNLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆RSDH | |
| | | | QRTHTGEKPYKCPECGKSFSRSD | | LTNHX₁₇X₁₈X₁₉HX₂₀X₂₁X₂₂X₂₃X₂₄X₈X₉CX₁₀X₁₁CX₁₂X₁₃X₁₄X₁₅X₁₆QR | |
| | | | HLTNHQRTHTGEKPYKCPECGKS | | AHLERHX₁₇X₁₈X₁₉HX₂₀X₂₁X₂₂X₂₃X₂₄X₂₅X₂₆X₂₇X₂₈X₂₉X₃₀ | |
| | | | FSQRAHLERHQRTHTGEKPTGKK | | | |
| | | | TS | | | |
| gtgggaggg | GTG GGA | 1121 | LEPGEKPYKCPECGKSFSRSDKL | 1670 | X₁X₂X₃X₄X₅X₆X₇X₈X₉CX₁₀X₁₁CX₁₂X₁₃X₁₄X₁₅X₁₆RSDKLVRHX₁₇X₁₈X₁₉ | 2219 |
| cggagggcg | ggg CGG | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDDLVRHX₁₇X₁₈ | |
| ggg | AGG GCG | | RSDDLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDHLTNHX₁₇ | |
| | GGG | | GKSFSRSDHLTNHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDKLTEH | |
| | | | KCPECGKSFSRSDKLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDKLV | |
| | | | GEKPYKCPECGKSFSRSDKLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆QRAH | |
| | | | QRTHTGEKPYKCPECGKSFSQRA | | LERHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | HLERHQRTHTGEKPYKCPECGKS | | DELVRHX₁₇X₁₈X₁₉HX₂₀X₂₁X₂₂X₂₃X₂₄X₂₅X₂₆X₂₇X₂₈X₂₉X₃₀ | |
| | | | FSRSDELVRHQRTHTGEKPTGKK | | | |
| | | | TS | | | |
| gcagtggga | GCA GTG | 1122 | LEPGEKPYKCPECGKSFSRSDDL | 1671 | X₁X₂X₃X₄X₅X₆X₇X₈X₉CX₁₀X₁₁CX₁₂X₁₃X₁₄X₁₅X₁₆RSDDLVRHX₁₇X₁₈X₁₉ | 2220 |
| gggcggagg | GGA ggg | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDHLTNHX₁₇X₁₈ | |
| gcg | CGG AGG | | RSDHLTNHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDKLTEHX₁₇ | |
| | GCG | | GKSFSRSDKLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDKLVRH | |
| | | | KCPECGKSFSRSDKLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QRAHLE | |
| | | | GEKPYKCPECGKSFSQRAHLERH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDE | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆QS | |
| | | | ELVRHQRTHTGEKPYKCPECGKS | | GDLRRHX₁₇X₁₈X₁₉HX₂₀X₂₁X₂₂X₂₃X₂₄X₂₅X₂₆X₂₇X₂₈X₂₉X₃₀ | |
| | | | FSQSGDLRRHQRTHTGEKPTGKK | | | |
| | | | TS | | | |
| ggagggcgg | GGA ggg | 1123 | LEPGEKPYKCPECGKSFSDCRDL | 1672 | X₁X₂X₃X₄X₅X₆X₇X₈X₉CX₁₀X₁₁CX₁₂X₁₃X₁₄X₁₅X₁₆DCRDLARHX₁₇X₁₈X₁₉ | 2221 |
| agggcgggg | CGG AGG | | ARHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDKLVRHX₁₇X₁₈ | |
| gcc | GCG GGG | | RSDKLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDDLVRHX₁₇ | |
| | GCC | | GKSFSRSDDLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDHLTNH | |
| | | | KCPECGKSFSRSDHLTNHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDKLT | |
| | | | GEKPYKCPECGKSFSRSDKLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆RSDK | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆QR | |
| | | | KLVRHQRTHTGEKPYKCPECGKS | | AHLERHX₁₇X₁₈X₁₉HX₂₀X₂₁X₂₂X₂₃X₂₄X₂₅X₂₆X₂₇X₂₈X₂₉X₃₀ | |
| | | | FSQRAHLERHQRTHTGEKPTGKK | | | |
| | | | TS | | | |
| cgcggcgtg | CGC GGC | 1124 | LEPGEKPYKCPECGKSFSQLAHL | 1673 | X₁X₂X₃X₄X₅X₆X₇X₈X₉CX₁₀X₁₁CX₁₂X₁₃X₁₄X₁₅X₁₆QLAHLRAHX₁₇X₁₈X₁₉ | 2222 |
| gaggcaggg | GTG GAG | | RAHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDKLVRHX₁₇X₁₈ | |
| aga | GCA ggg | | RSDKLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QSGDLRRHX₁₇ | |
| | AGA | | GKSFSQSGDLRRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDNLVRH | |
| | | | KCPECGKSFSRSDNLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDELV | |
| | | | GEKPYKCPECGKSFSRSDELVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆DPGH | |
| | | | QRTHTGEKPYKCPECGKSFSDPG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆HT | |
| | | | HLVRHQRTHTGEKPYKCPECGKS | | GHLLEHX₁₇X₁₈X₁₉HX₂₀X₂₁X₂₂X₂₃X₂₄X₂₅X₂₆X₂₇X₂₈X₂₉X₃₀ | |
| | | | FSHTGHLLEHQRTHTGEKPTGKK | | | |
| | | | TS | | | |
| ggccgcggc | GGC CGC | 1125 | LEPGEKPYKCPECGKSFSRSDKL | 1674 | X₁X₂X₃X₄X₅X₆X₇X₈X₉CX₁₀X₁₁CX₁₂X₁₃X₁₄X₁₅X₁₆RSDKLVRHX₁₇X₁₈X₁₉ | 2223 |
| gtggaggca | GGC GTG | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QSGDLRRHX₁₇X₁₈ | |
| ggg | GAG GCA | | QSGDLRRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDNLVRHX₁₇ | |
| | GGG | | GKSFSRSDNLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDELVRH | |
| | | | KCPECGKSFSRSDELVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DPGHLV | |
| | | | GEKPYKCPECGKSFSDPGHLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆HTGH | |
| | | | QRTHTGEKPYKCPECGKSFSHTG | | LLEHX₁₇X₁₈X₁₉HX₂₀X₂₁X₂₂X₂₃X₂₄X₈X₉CX₁₀X₁₁CX₁₂X₁₃X₁₄X₁₅X₁₆DP | |
| | | | HLLEHQRTHTGEKPYKCPECGKS | | GHLVRHX₁₇X₁₈X₁₉HX₂₀X₂₁X₂₂X₂₃X₂₄X₂₅X₂₆X₂₇X₂₈X₂₉X₃₀ | |
| | | | FSDPGHLVRHQRTHTGEKPTGKK | | | |
| | | | TS | | | |
| ggcgtggag | GGC GTG | 1126 | LEPGEKPYKCPECGKSFSRRDEL | 1675 | X₁X₂X₃X₄X₅X₆X₇X₈X₉CX₁₀X₁₁CX₁₂X₁₃X₁₄X₁₅X₁₆RRDELNVHX₁₇X₁₈X₁₉ | 2224 |
| gcagggaga | GAG GCA | | NVHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QLAHLRAHX₁₇X₁₈ | |
| atg | ggg AGA | | QLAHLRAHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDKLVRHX₁₇ | |
| | ATG | | GKSFSRSDKLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QSGDLRRH | |
| | | | KCPECGKSFSQSGDLRRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDNLV | |
| | | | GEKPYKCPECGKSFSRSDNLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDE | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆DP | |
| | | | ELVRHQRTHTGEKPYKCPECGKS | | GHLVRHX₁₇X₁₈X₁₉HX₂₀X₂₁X₂₂X₂₃X₂₄X₂₅X₂₆X₂₇X₂₈X₂₉X₃₀ | |
| | | | FSDPGHLVRHQRTHTGEKPTGKK | | | |
| | | | TS | | | |
| gtggaggca | GTG GAG | 1127 | LEPGEKPYKCPECGKSFSQSGHL | 1676 | X₁X₂X₃X₄X₅X₆X₇X₈X₉CX₁₀X₁₁CX₁₂X₁₃X₁₄X₁₅X₁₆QSGHLTEHX₁₇X₁₈X₁₉ | 2225 |
| gggagaatg | GCA ggg | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RRDELNVHX₁₇X₁₈ | |
| cga | AGA ATG | | RRDELNVHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QLAHLRAHX₁₇ | |
| | CGA | | GKSFSQLAHLRAHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDKLVRH | |
| | | | KCPECGKSFSRSDKLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QSGDLR | |
| | | | GEKPYKCPECGKSFSQSGDLRRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDN | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | NLVRHQRTHTGEKPYKCPECGKS | | DELVRHX₁₇X₁₈X₁₉HX₂₀X₂₁X₂₂X₂₃X₂₄X₂₅X₂₆X₂₇X₂₈X₂₉X₃₀ | |
| | | | FSRSDELVRHQRTHTGEKPTGKK | | | |
| | | | TS | | | |
| ggggtgggc | GGG GTG | 1128 | LEPGEKPYKCPECGKSFSRSDHL | 1677 | X₁X₂X₃X₄X₅X₆X₇X₈X₉CX₁₀X₁₁CX₁₂X₁₃X₁₄X₁₅X₁₆RSDHLTNHX₁₇X₁₈X₁₉ | 2226 |
| gcccagggt | GGC GCC | | TNHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSGHLVRHX₁₇X₁₈ | |
| agg | CAG GGT | | TSGHLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RADNLTEHX₁₇ | |
| | AGG | | GKSFSRADNLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DCRDLARH | |
| | | | KCPECGKSFSDCRDLARHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DPGHLV | |
| | | | GEKPYKCPECGKSFSDPGHLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDE | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | ELVRHQRTHTGEKPYKCPECGKS | | DKLVRHX₁₇X₁₈X₁₉HX₂₀X₂₁X₂₂X₂₃X₂₄X₂₅X₂₆X₂₇X₂₈X₂₉X₃₀ | |
| | | | FSRSDKLVRHQRTHTGEKPTGKK | | | |
| | | | TS | | | |
| gtgggcgcc | GTG GGC | 1129 | LEPGEKPYKCPECGKSFSQSGDL | 1678 | X₁X₂X₃X₄X₅X₆X₇X₈X₉CX₁₀X₁₁CX₁₂X₁₃X₁₄X₁₅X₁₆QSGDLRRHX₁₇X₁₈X₁₉ | 2227 |
| cagggtagg | GCC CAG | | RRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDHLTNHX₁₇X₁₈ | |
| gca | GGT AGG | | RSDHLTNHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSGHLVRHX₁₇ | |
| | GCA | | GKSFSTSGHLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RADNLTEH | |
| | | | KCPECGKSFSRADNLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DCRDLA | |
| | | | GEKPYKCPECGKSFSDCRDLARH | | RHX₁₇X₁₈X₁₉HX₂₀X₂₁X₂₂X₂₃X₂₄X₈X₉CX₁₀X₁₁CX₁₂X₁₃X₁₄X₁₅X₁₆DPGH | |
| | | | QRTHTGEKPYKCPECGKSFSDPG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | HLVRHQRTHTGEKPYKCPECGKS | | DELVRHX₁₇X₁₈X₁₉HX₂₀X₂₁X₂₂X₂₃X₂₄X₂₅X₂₆X₂₇X₂₈X₂₉X₃₀ | |
| | | | FSRSDELVRHQRTHTGEKPTGKK | | | |
| | | | TS | | | |
| gcaggtggc | GCA GGT | 1130 | LEPGEKPYKCPECGKSFSRSDNL | 1679 | X₁X₂X₃X₄X₅X₆X₇X₈X₉CX₁₀X₁₁CX₁₂X₁₃X₁₄X₁₅X₁₆RSDNLVRHX₁₇X₁₈X₁₉ | 2228 |
| cgcggcgtg | GGC CGC | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDELVRHX₁₇X₁₈ | |
| gag | GGC GTG | | RSDELVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DPGHLVRHX₁₇ | |
| | GAG | | GKSFSDPGHLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆HTGHLLEH | |
| | | | KCPECGKSFSHTGHLLEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DPGHLV | |
| | | | GEKPYKCPECGKSFSDPGHLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆TSGH | |
| | | | QRTHTGEKPYKCPECGKSFSTSG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆QS | |
| | | | HLVRHQRTHTGEKPYKCPECGKS | | GDLRRHX₁₇X₁₈X₁₉HX₂₀X₂₁X₂₂X₂₃X₂₄X₂₅X₂₆X₂₇X₂₈X₂₉X₃₀ | |
| | | | FSQSGDLRRHQRTHTGEKPTGKK | | | |
| | | | TS | | | |
| ggtggccgc | GGT GGC | 1131 | LEPGEKPYKCPECGKSFSQSGDL | 1680 | X₁X₂X₃X₄X₅X₆X₇X₈X₉CX₁₀X₁₁CX₁₂X₁₃X₁₄X₁₅X₁₆QSGDLRRHX₁₇X₁₈X₁₉ | |
| ggcgtggag | CGC GGC | | RRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDNLVRHX₁₇X₁₈ | 2229 |
| gca | GTG GAG | | RSDNLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDELVRHX₁₇ | |
| | GCA | | GKSFSRSDELVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DPGHLVRH | |
| | | | KCPECGKSFSDPGHLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆HTGHLL | |
| | | | GEKPYKCPECGKSFSHTGHLLEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆DPGH | |
| | | | QRTHTGEKPYKCPECGKSFSDPG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | HLVRHQRTHTGEKPYKCPECGKS | | GHLVRHX₁₇X₁₈X₁₉HX₂₀X₂₁X₂₂X₂₃X₂₄X₂₅X₂₆X₂₇X₂₈X₂₉X₃₀ | |
| | | | FSTSGHLVRHQRTHTGEKPTGKK | | | |
| | | | TS | | | |
| agggcaggt | AGG GCA | 1132 | LEPGEKPYKCPECGKSFSRSDEL | 1681 | X₁X₂X₃X₄X₅X₆X₇X₈X₉CX₁₀X₁₁CX₁₂X₁₃X₁₄X₁₅X₁₆RSDELVRHX₁₇X₁₈X₁₉ | 2230 |
| ggccgcggc | GGT GGC | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆DPGHLVRHX₁₇X₁₈ | |
| gtg | CGC GGC | | DPGHLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆HTGHLLEHX₁₇ | |
| | GTG | | GKSFSHTGHLLEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DPGHLVRH | |
| | | | KCPECGKSFSDPGHLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSGHLV | |
| | | | GEKPYKCPECGKSFSTSGHLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆QSGD | |
| | | | QRTHTGEKPYKCPECGKSFSQSG | | LRRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | DLRRHQRTHTGEKPYKCPECGKS | | DHLTNHX₁₇X₁₈X₁₉HX₂₀X₂₁X₂₂X₂₃X₂₄X₂₅X₂₆X₂₇X₂₈X₂₉X₃₀ | |
| | | | FSRSDHLTNHQRTHTGEKPTGKK | | | |
| | | | TS | | | |
| gcccagggt | GCC CAG | 1133 | LEPGEKPYKCPECGKSFSDPGHL | 1682 | X₁X₂X₃X₄X₅X₆X₇X₈X₉CX₁₀X₁₁CX₁₂X₁₃X₁₄X₁₅X₁₆DPGHLVRHX₁₇X₁₈X₁₉ | 2231 |
| agggcaggt | GGT AGG | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSGHLVRHX₁₇X₁₈ | |
| ggc | GCA GGT | | TSGHLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QSGDLRRHX₁₇ | |
| | GGC | | GKSFSQSGDLRRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDHLTNH | |
| | | | KCPECGKSFSRSDHLTNHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSGHLV | |
| | | | GEKPYKCPECGKSFSTSGHLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RADN | |
| | | | QRTHTGEKPYKCPECGKSFSRAD | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆DC | |
| | | | NLTEHQRTHTGEKPYKCPECGKS | | RDLARHX₁₇X₁₈X₁₉HX₂₀X₂₁X₂₂X₂₃X₂₄X₂₅X₂₆X₂₇X₂₈X₂₉X₃₀ | |
| | | | FSDCRDLARHQRTHTGEKPTGKK | | | |
| | | | TS | | | |
| ggcgcccag | GGC GCC | 1134 | LEPGEKPYKCPECGKSFSTSGHL | 1683 | X₁X₂X₃X₄X₅X₆X₇X₈X₉CX₁₀X₁₁CX₁₂X₁₃X₁₄X₁₅X₁₆TSGHLVRHX₁₇X₁₈X₁₉ | 2232 |
| ggtagggca | CAG GGT | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QSGDLRRHX₁₇X₁₈ | |
| ggt | AGG GCA | | QSGDLRRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDHLTNHX₁₇ | |
| | GGT | | GKSFSRSDHLTNHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSGHLVRH | |
| | | | KCPECGKSFSTSGHLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RADNLT | |
| | | | GEKPYKCPECGKSFSRADNLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆DCRD | |
| | | | QRTHTGEKPYKCPECGKSFSDCR | | LARHX₁₇X₁₈X₁₉HX₂₀X₂₁X₂₂X₂₃X₂₄X₈X₉CX₁₀X₁₁CX₁₂X₁₃X₁₄X₁₅X₁₆DP | |
| | | | DLARHQRTHTGEKPYKCPECGKS | | GHLVRHX₁₇X₁₈X₁₉HX₂₀X₂₁X₂₂X₂₃X₂₄X₂₅X₂₆X₂₇X₂₈X₂₉X₃₀ | |
| | | | FSDPGHLVRHQRTHTGEKPTGKK | | | |
| | | | TS | | | |
| cagggtagg | CAG GGT | 1135 | LEPGEKPYKCPECGKSFSHTGHL | 1684 | X₁X₂X₃X₄X₅X₆X₇X₈X₉CX₁₀X₁₁CX₁₂X₁₃X₁₄X₁₅X₁₆HTGHLLEHX₁₇X₁₈X₁₉ | 2233 |
| gcaggtggc | AGG GCA | | LEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆DPGHLVRHX₁₇X₁₈ | |
| cgc | GGT GGC | | DPGHLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSGHLVRHX₁₇ | |
| | CGC | | GKSFSTSGHLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QSGDLRRH | |
| | | | KCPECGKSFSQSGDLRRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDHLT | |
| | | | GEKPYKCPECGKSFSRSDHLTNH | | NHX₁₇X₁₈X₁₉HX₂₀X₂₁X₂₂X₂₃X₂₄X₈X₉CX₁₀X₁₁CX₁₂X₁₃X₁₄X₁₅X₁₆TSGH | |
| | | | QRTHTGEKPYKCPECGKSFSTSG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RA | |
| | | | HLVRHQRTHTGEKPYKCPECGKS | | DNLTEHX₁₇X₁₈X₁₉HX₂₀X₂₁X₂₂X₂₃X₂₄X₂₅X₂₆X₂₇X₂₈X₂₉X₃₀ | |
| | | | FSRADNLTEHQRTHTGEKPTGKK | | | |
| | | | TS | | | |
| ggtagggca | GGT AGG | 1136 | LEPGEKPYKCPECGKSFSDPGHL | 1685 | X₁X₂X₃X₄X₅X₆X₇X₈X₉CX₁₀X₁₁CX₁₂X₁₃X₁₄X₁₅X₁₆DPGHLVRHX₁₇X₁₈X₁₉ | 2234 |
| ggtggccgc | GCA GGT | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆HTGHLLEHX₁₇X₁₈ | |
| ggc | GGC CGC | | HTGHLLEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DPGHLVRHX₁₇ | |
| | GGC | | GKSFSDPGHLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSGHLVRH | |
| | | | KCPECGKSFSTSGHLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QSGDLR | |
| | | | GEKPYKCPECGKSFSQSGDLRRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDH | |
| | | | QRTHTGEKPYKCPECGKSFSRSD | | VLTNHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | HLTNHQRTHTGEKPYKCPECGKS | | GHLVRHX₁₇X₁₈X₁₉HX₂₀X₂₁X₂₂X₂₃X₂₄X₂₅X₂₆X₂₇X₂₈X₂₉X₃₀ | |
| | | | FSTSGHLVRHQRTHTGEKPTGKK | | | |
| | | | TS | | | |
| agggtaggg | AGG GTA | 1137 | LEPGEKPYKCPECGKSFSRSDDL | 1686 | X₁X₂X₃X₄X₅X₆X₇X₈X₉CX₁₀X₁₁CX₁₂X₁₃X₁₄X₁₅X₁₆RSDDLVRHX₁₇X₁₈X₁₉ | 2235 |
| caggtggcc | ggg CAG | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆DCRDLARHX₁₇X₁₈ | |
| gcg | GTG GCC | | DCRDLARHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDELVRHX₁₇ | |
| | GCG | | GKSFSRSDELVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RADNLTEH | |
| | | | KCPECGKSFSRADNLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDKLV | |
| | | | GEKPYKCPECGKSFSRSDKLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆QSSS | |
| | | | QRTHTGEKPYKCPECGKSFSQSS | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | SLVRHQRTHTGEKPYKCPECGKS | | DHLTNHX₁₇X₁₈X₁₉HX₂₀X₂₁X₂₂X₂₃X₂₄X₂₅X₂₆X₂₇X₂₈X₂₉X₃₀ | |
| | | | FSRSDHLTNHQRTHTGEKPTGKK | | | |
| | | | TS | | | |
| cccagggta | CCC AGG | 1138 | LEPGEKPYKCPECGKSFSDCRDL | 1687 | X₁X₂X₃X₄X₅X₆X₇X₈X₉CX₁₀X₁₁CX₁₂X₁₃X₁₄X₁₅X₁₆DCRDLARHX₁₇X₁₈X₁₉ | 2236 |
| gggcaggtg | GTA ggg | | ARHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDELVRHX₁₇X₁₈ | |
| gcc | CAG GTG | | RSDELVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RADNLTEHX₁₇ | |
| | GCC | | GKSFSRADNLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDKLVRH | |
| | | | KCPECGKSFSRSDKLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QSSSLV | |
| | | | GEKPYKCPECGKSFSQSSSLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDH | |
| | | | QRTHTGEKPYKCPECGKSFSRSD | | LTNHX₁₇X₁₈X₁₉HX₂₀X₂₁X₂₂X₂₃X₂₄X₈X₉CX₁₀X₁₁CX₁₂X₁₃X₁₄X₁₅X₁₆SK | |
| | | | HLTNHQRTHTGEKPYKCPECGKS | | KHLAEHX₁₇X₁₈X₁₉HX₂₀X₂₁X₂₂X₂₃X₂₄X₂₅X₂₆X₂₇X₂₈X₂₉X₃₀ | |
| | | | FSSKKHLAEHQRTHTGEKPTGKK | | | |
| | | | TS | | | |
| gtagggcag | GTA ggg | 1139 | LEPGEKPYKCPECGKSFSRSDDL | 1688 | X₁X₂X₃X₄X₅X₆X₇X₈X₉CX₁₀X₁₁CX₁₂X₁₃X₁₄X₁₅X₁₆RSDDLVRHX₁₇X₁₈X₁₉ | 2237 |
| gtggccgcg | CAG GTG | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDDLVRHX₁₇X₁₈ | |
| gcg | GCC GCG | | RSDDLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DCRDLARHX₁₇ | |
| | GCG | | GKSFSDCRDLARHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDELVRH | |
| | | | KCPECGKSFSRSDELVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RADNLT | |
| | | | GEKPYKCPECGKSFSRADNLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆RSDK | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆QS | |
| | | | KLVRHQRTHTGEKPYKCPECGKS | | SSLVRHX₁₇X₁₈X₁₉HX₂₀X₂₁X₂₂X₂₃X₂₄X₂₅X₂₆X₂₇X₂₈X₂₉X₃₀ | |
| | | | FSQSSSLVRHQRTHTGEKPTGKK | | | |
| | | | TS | | | |
| gggcaggtg | ggg CAG | 1140 | LEPGEKPYKCPECGKSFSRSDHL | 1689 | X₁X₂X₃X₄X₅X₆X₇X₈X₉CX₁₀X₁₁CX₁₂X₁₃X₁₄X₁₅X₁₆RSDHLTTHX₁₇X₁₈X₁₉ | 2238 |
| gccgcggcg | GTG GCC | | TTHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDDLVRHX₁₇X₁₈ | |
| tgg | GCG GCG | | RSDDLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDDLVRHX₁₇ | |
| | TGG | | GKSFSRSDDLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DCRDLARH | |
| | | | KCPECGKSFSDCRDLARHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDELV | |
| | | | GEKPYKCPECGKSFSRSDELVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RADN | |
| | | | QRTHTGEKPYKCPECGKSFSRAD | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | NLTEHQRTHTGEKPYKCPECGKS | | DKLVRHX₁₇X₁₈X₁₉HX₂₀X₂₁X₂₂X₂₃X₂₄X₂₅X₂₆X₂₇X₂₈X₂₉X₃₀ | |
| | | | FSRSDKLVRHQRTHTGEKPTGKK | | | |
| | | | TS | | | |
| gtggccgcg | GTG GCC | 1141 | LEPGEKPYKCPECGKSFSRADNL | 1690 | X₁X₂X₃X₄X₅X₆X₇X₈X₉CX₁₀X₁₁CX₁₂X₁₃X₁₄X₁₅X₁₆RADNLTEHX₁₇X₁₈X₁₉ | 2239 |
| gcgtggagg | GCG GCG | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDHLTNHX₁₇X₁₈ | |
| cag | TGG AGG | | RSDHLTNHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDHLTTHX₁₇ | |
| | CAG | | GKSFSRSDHLTTHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDDLVRH | |
| | | | KCPECGKSFSRSDDLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDDLV | |
| | | | GEKPYKCPECGKSFSRSDDLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆DCRD | |
| | | | QRTHTGEKPYKCPECGKSFSDCR | | LARHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | DLARHQRTHTGEKPYKCPECGKS | | DELVRHX₁₇X₁₈X₁₉HX₂₀X₂₁X₂₂X₂₃X₂₄X₂₅X₂₆X₂₇X₂₈X₂₉X₃₀ | |
| | | | FSRSDELVRHQRTHTGEKPTGKK | | | |
| | | | TS | | | |
| caggtggcc | CAG GTG | 1142 | LEPGEKPYKCPECGKSFSRSDHL | 1691 | X₁X₂X₃X₄X₅X₆X₇X₈X₉CX₁₀X₁₁CX₁₂X₁₃X₁₄X₁₅X₁₆RSDHLTNHX₁₇X₁₈X₁₉ | 2240 |
| gcggcgtgg | GCC GCG | | TNHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDHLTTHX₁₇X₁₈ | |
| agg | GCG TGG | | RSDHLTTHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDDLVRHX₁₇ | |
| | AGG | | GKSFSRSDDLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDDLVRH | |
| | | | KCPECGKSFSRSDDLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DCRDLA | |
| | | | GEKPYKCPECGKSFSDCRDLARH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDE | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RA | |
| | | | ELVRHQRTHTGEKPYKCPECGKS | | DNLTEHX₁₇X₁₈X₁₉HX₂₀X₂₁X₂₂X₂₃X₂₄X₂₅X₂₆X₂₇X₂₈X₂₉X₃₀ | |
| | | | FSRADNLTEHQRTHTGEKPTGKK | | | |
| | | | TS | | | |
| gcgcccagg | gcg CCC | 1143 | LEPGEKPYKCPECGKSFSRSDEL | 1692 | X₁X₂X₃X₄X₅X₆X₇X₈X₉CX₁₀X₁₁CX₁₂X₁₃X₁₄X₁₅X₁₆RSDELVRHX₁₇X₁₈X₁₉ | 2241 |
| gtagggcag | AGG GTA | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RADNLTEHX₁₇X₁₈ | |
| gtg | ggg CAG | | RADNLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDKLVRHX₁₇ | |
| | GTG | | GKSFSRSDKLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QSSSLVRH | |
| | | | KCPECGKSFSQSSSLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDHLT | |
| | | | GEKPYKCPECGKSFSRSDHLTNH | | NHX₁₇X₁₈X₁₉HX₂₀X₂₁X₂₂X₂₃X₂₄X₈X₉CX₁₀X₁₁CX₁₂X₁₃X₁₄X₁₅X₁₆SKKH | |
| | | | QRTHTGEKPYKCPECGKSFSSKK | | LAEHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | HLAEHQRTHTGEKPYKCPECGKS | | DDLVRHX₁₇X₁₈X₁₉HX₂₀X₂₁X₂₂X₂₃X₂₄X₂₅X₂₆X₂₇X₂₈X₂₉X₃₀ | |
| | | | FSRSDDLVRHQRTHTGEKPTGKK | | | |
| | | | TS | | | |
| gggtgggcg | GGG TGG | 1144 | LEPGEKPYKCPECGKSFSRSDKL | 1693 | X₁X₂X₃X₄X₅X₆X₇X₈X₉CX₁₀X₁₁CX₁₂X₁₃X₁₄X₁₅X₁₆RSDKLVRHX₁₇X₁₈X₁₉ | 2242 |
| cccagggta | gcg CCC | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QSSSLVRHX₁₇X₁₈ | |
| ggg | AGG GTA | | QSSSLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDHLTNHX₁₇ | |
| | GGG | | GKSFSRSDHLTNHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆SKKHLAEH | |
| | | | KCPECGKSFSSKKHLAEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDDLV | |
| | | | GEKPYKCPECGKSFSRSDDLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDH | |
| | | | QRTHTGEKPYKCPECGKSFSRSD | | LTTHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | HLTTHQRTHTGEKPYKCPECGKS | | DKLVRHX₁₇X₁₈X₁₉HX₂₀X₂₁X₂₂X₂₃X₂₄X₂₅X₂₆X₂₇X₂₈X₂₉X₃₀ | |
| | | | FSRSDKLVRHQRTHTGEKPTGKK | | | |
| | | | TS | | | |
| tgggcgccc | TGG gcg | 1145 | LEPGEKPYKCPECGKSFSRADNL | 1694 | X₁X₂X₃X₄X₅X₆X₇X₈X₉CX₁₀X₁₁CX₁₂X₁₃X₁₄X₁₅X₁₆RADNLTEHX₁₇X₁₈X₁₉ | 2243 |
| agggtaggg | CCC AGG | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDKLVRHX₁₇X₁₈ | |
| cag | GTA ggg | | RSDKLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QSSSLVRHX₁₇ | |
| | CAG | | GKSFSQSSSLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDHLTNH | |
| | | | KCPECGKSFSRSDHLTNHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆SKKHLA | |
| | | | GEKPYKCPECGKSFSSKKHLAEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆RSDD | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | DLVRHQRTHTGEKPYKCPECGKS | | DHLTTHX₁₇X₁₈X₁₉HX₂₀X₂₁X₂₂X₂₃X₂₄X₂₅X₂₆X₂₇X₂₈X₂₉X₃₀ | |
| | | | FSRSDHLTTHQRTHTGEKPTGKK | | | |
| | | | TS | | | |
| gcggcgtgg | GCG GCG | 1146 | LEPGEKPYKCPECGKSFSQSSNL | 1695 | X₁X₂X₃X₄X₅X₆X₇X₈X₉CX₁₀X₁₁CX₁₂X₁₃X₁₄X₁₅X₁₆QSSNLVRHX₁₇X₁₈X₁₉ | 2244 |
| aggcaggga | TGG AGG | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QRAHLERHX₁₇X₁₈ | |
| gaa | CAG GGA | | QRAHLERHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RADNLTEHX₁₇ | |
| | GAA | | GKSFSRADNLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDHLTNH | |
| | | | KCPECGKSFSRSDHLTNHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDHLT | |
| | | | GEKPYKCPECGKSFSRSDHLTTH | | THX₁₇X₁₈X₁₉HX₂₀X₂₁X₂₂X₂₃X₂₄X₈X₉CX₁₀X₁₁CX₁₂X₁₃X₁₄X₁₅X₁₆RSDD | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | DLVRHQRTHTGEKPYKCPECGKS | | DDLVRHX₁₇X₁₈X₁₉HX₂₀X₂₁X₂₂X₂₃X₂₄X₂₅X₂₆X₂₇X₂₈X₂₉X₃₀ | |
| | | | FSRSDDLVRHQRTHTGEKPTGKK | | | |
| | | | TS | | | |
| gccgcggcg | GCC GCG | 1147 | LEPGEKPYKCPECGKSFSQRAHL | 1696 | X₁X₂X₃X₄X₅X₆X₇X₈X₉CX₁₀X₁₁CX₁₂X₁₃X₁₄X₁₅X₁₆QRAHLERHX₁₇X₁₈X₁₉ | 2245 |
| tggaggcag | GCG TGG | | ERHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RADNLTEHX₁₇X₁₈ | |
| gga | AGG CAG | | RADNLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDHLTNHX₁₇ | |
| | GGA | | GKSFSRSDHLTNHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDHLTTH | |
| | | | KCPECGKSFSRSDHLTTHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDDLV | |
| | | | GEKPYKCPECGKSFSRSDDLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDD | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆DC | |
| | | | DLVRHQRTHTGEKPYKCPECGKS | | RDLARHX₁₇X₁₈X₁₉HX₂₀X₂₁X₂₂X₂₃X₂₄X₂₅X₂₆X₂₇X₂₈X₂₉X₃₀ | |
| | | | FSDCRDLARHQRTHTGEKPTGKK | | | |
| | | | TS | | | |
| aaccctcgt | AAC CCT | 1148 | LEPGEKPYKCPECGKSFSTSGNL | 1697 | X₁X₂X₃X₄X₅X₆X₇X₈X₉CX₁₀X₁₁CX₁₂X₁₃X₁₄X₁₅X₁₆TSGNLTEHX₁₇X₁₈X₁₉ | 2246 |
| cgacatgga | CGT CGA | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QRAHLERHX₁₇X₁₈ | |
| cat | CAT GGA | | QRAHLERHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSGNLTEHX₁₇ | |
| | CAT | | GKSFSTSGNLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QSGHLTEH | |
| | | | KCPECGKSFSQSGHLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆SRRTCR | |
| | | | GEKPYKCPECGKSFSSRRTCRAH | | AHX₁₇X₁₈X₁₉HX₂₀X₂₁X₂₂X₂₃X₂₄X₈X₉CX₁₀X₁₁CX₁₂X₁₃X₁₄X₁₅X₁₆TKNS | |
| | | | QRTHTGEKPYKCPECGKSFSTKN | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆DS | |
| | | | SLTEHQRTHTGEKPYKCPECGKS | | GNLRVHX₁₇X₁₈X₁₉HX₂₀X₂₁X₂₂X₂₃X₂₄X₂₅X₂₆X₂₇X₂₈X₂₉X₃₀ | |
| | | | FSDSGNLRVHQRTHTGEKPTGKK | | | |
| | | | TS | | | |
| cctcgtcga | CCT CGT | 1149 | LEPGEKPYKCPECGKSFSDPGHL | 1698 | X₁X₂X₃X₄X₅X₆X₇X₈X₉CX₁₀X₁₁CX₁₂X₁₃X₁₄X₁₅X₁₆DPGHLVRHX₁₇X₁₈X₁₉ | 2247 |
| catggacat | CGA CAT | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSGNLTEHX₁₇X₁₈ | |
| ggc | GGA CAT | | TSGNLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QRAHLERHX₁₇ | |
| | GGC | | GKSFSQRAHLERHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSGNLTEH | |
| | | | KCPECGKSFSTSGNLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QSGHLT | |
| | | | GEKPYKCPECGKSFSQSGHLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆SRRT | |
| | | | QRTHTGEKPYKCPECGKSFSSRR | | CRAHX₁₇X₁₈X₁₉HX₂₀X₂₁X₂₂X₂₃X₂₄X₈X₉CX₁₀X₁₁CX₁₂X₁₃X₁₄X₁₅X₁₆TK | |
| | | | TCRAHQRTHTGEKPYKCPECGKS | | NSLTEHX₁₇X₁₈X₁₉HX₂₀X₂₁X₂₂X₂₃X₂₄X₂₅X₂₆X₂₇X₂₈X₂₉X₃₀ | |
| | | | FSTKNSLTEHQRTHTGEKPTGKK | | | |
| | | | TS | | | |
| catggacat | CAT GGA | 1150 | LEPGEKPYKCPECGKSFSRADNL | 1699 | X₁X₂X₃X₄X₅X₆X₇X₈X₉CX₁₀X₁₁CX₁₂X₁₃X₁₄X₁₅X₁₆RADNLTEHX₁₇X₁₈X₁₉ | 2248 |
| ggccgacta | CAT GGC | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QNSTLTEHX₁₇X₁₈ | |
| cag | CGA CTA | | QNSTLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QSGHLTEHX₁₇ | |
| | CAG | | GKSFSQSGHLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DPGHLVRH | |
| | | | KCPECGKSFSDPGHLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSGNLT | |
| | | | GEKPYKCPECGKSFSTSGNLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆QRAH | |
| | | | QRTHTGEKPYKCPECGKSFSQRA | | LERHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | HLERHQRTHTGEKPYKCPECGKS | | GNLTEHX₁₇X₁₈X₁₉HX₂₀X₂₁X₂₂X₂₃X₂₄X₂₅X₂₆X₂₇X₂₈X₂₉X₃₀ | |
| | | | FSTSGNLTEHQRTHTGEKPTGKK | | | |
| | | | TS | | | |
| caaaaccct | CAA AAC | 1151 | LEPGEKPYKCPECGKSFSQRAHL | 1700 | X₁X₂X₃X₄X₅X₆X₇X₈X₉CX₁₀X₁₁CX₁₂X₁₃X₁₄X₁₅X₁₆QRAHLERHX₁₇X₁₈X₁₉ | 2249 |
| cgtcgacat | CCT CGT | | ERHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSGNLTEHX₁₇X₁₈ | |
| gga | CGA CAT | | TSGNLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QSGHLTEHX₁₇ | |
| | GGA | | GKSFSQSGHLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆SRRTCRAH | |
| | | | KCPECGKSFSSRRTCRAHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TKNSLT | |
| | | | GEKPYKCPECGKSFSTKNSLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆DSGN | |
| | | | QRTHTGEKPYKCPECGKSFSDSG | | LRVHX₁₇X₁₈X₁₉HX₂₀X₂₁X₂₂X₂₃X₂₄X₈X₉CX₁₀X₁₁CX₁₂X₁₃X₁₄X₁₅X₁₆QS | |
| | | | NLRVHQRTHTGEKPYKCPECGKS | | GNLTEHX₁₇X₁₈X₁₉HX₂₀X₂₁X₂₂X₂₃X₂₄X₂₅X₂₆X₂₇X₂₈X₂₉X₃₀ | |
| | | | FSQSGNLTEHQRTHTGEKPTGKK | | | |
| | | | TS | | | |
| cgtcgacat | CGT CGA | 1152 | LEPGEKPYKCPECGKSFSQSGHL | 1701 | X₁X₂X₃X₄X₅X₆X₇X₈X₉CX₁₀X₁₁CX₁₂X₁₃X₁₄X₁₅X₁₆QSGHLTEHX₁₇X₁₈X₁₉ | 2250 |
| ggacatggc | CAT GGA | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆DPGHLVRHX₁₇X₁₈ | |
| cga | CAT GGC | | DPGHLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSGNLTEHX₁₇ | |
| | CGA | | GKSFSTSGNLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QRAHLERH | |
| | | | KCPECGKSFSQRAHLERHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSGNLT | |
| | | | GEKPYKCPECGKSFSTSGNLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆QSGH | |
| | | | QRTHTGEKPYKCPECGKSFSQSG | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆SR | |
| | | | HLTEHQRTHTGEKPYKCPECGKS | | RTCRAHX₁₇X₁₈X₁₉HX₂₀X₂₁X₂₂X₂₃X₂₄X₂₅X₂₆X₂₇X₂₈X₂₉X₃₀ | |
| | | | FSSRRTCRAHQRTHTGEKPTGKK | | | |
| | | | TS | | | |
| cgacatgga | CGA CAT | 1153 | LEPGEKPYKCPECGKSFSQNSTL | 1702 | XX₂X₃X₄X₅X₆X₇X₈X₉CX₁₀X₁₁CX₁₂X₁₃X₁₄X₁₅X₁₆QNSTLTEHX₁₇X₁₈X₁₉ | 2251 |
| catggccga | GGA CAT | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QSGHLTEHX₁₇X₁₈ | |
| cta | GGC CGA | | QSGHLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DPGHLVRHX₁₇ | |
| | CTA | | GKSFSDPGHLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSGNLTEH | |
| | | | KCPECGKSFSTSGNLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QRAHLE | |
| | | | GEKPYKCPECGKSFSQRAHLERH | | RHX₁₇X₁₈X₁₉HX₂₀X₂₁X₂₂X₂₃X₂₄X₈X₉CX₁₀X₁₁CX₁₂X₁₃X₁₄X₁₅X₁₆TSGN | |
| | | | QRTHTGEKPYKCPECGKSFSTSG | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆QS | |
| | | | NLTEHQRTHTGEKPYKCPECGKS | | GHLTEHX₁₇X₁₈KX₁₉HX₂₀X₂₁X₂₂X₂₃X₂₄X₂₅X₂₆X₂₇X₂₈X₂₉X₃₀ | |
| | | | FSQSGHLTEHQRTHTGEKPTGKK | | | |
| | | | TS | | | |
| gtcgacatg | GTC GAC | 1154 | LEPGEKPYKCPECGKSFSDPGNL | 1703 | X₁X₂X₃X₄X₅X₆X₇X₈X₉CX₁₀X₁₁CX₁₂X₁₃X₁₄X₁₅X₁₆DPGNLVRHX₁₇X₁₈X₁₉ | 2252 |
| gacatggcc | ATG GAC | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆DCRDLARHX₁₇X₁₈ | |
| gac | ATG GCC | | DCRDLARHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RRDELNVHX₁₇ | |
| | GAC | | GKSFSRRDELNVHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DPGNLVRH | |
| | | | KCPECGKSFSDPGNLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RRDELN | |
| | | | GEKPYKCPECGKSFSRRDELNVH | | VHX₁₇X₁₈X₁₉HX₂₀X₂₁X₂₂X₂₃X₂₄X₈X₉CX₁₀X₁₁CX₁₂X₁₃X₁₄X₁₅X₁₆DPGN | |
| | | | QRTHTGEKPYKCPECGKSFSDPG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆DP | |
| | | | NLVRHQRTHTGEKPYKCPECGKS | | GALVRHX₁₇X₁₈X₁₉HX₂₀X₂₁X₂₂X₂₃X₂₄X₂₅X₂₆X₂₇X₂₈X₂₉X₃₀ | |
| | | | FSDPGALVRHQRTHTGEKPTGKK | | | |
| | | | TS | | | |
| gtgctgcac | gtg CTG | 1155 | LEPGEKPYKCPECGKSFSTKNSL | 1704 | X₁X₂X₃X₄X₅X₆X₇X₈X₉CX₁₀X₁₁CX₁₂X₁₃X₁₄X₁₅X₁₆TKNSLTEHX₁₇X₁₈X₁₉ | 2253 |
| tggacccag | CAC TGG | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RADNLTEHX₁₇X₁₈ | |
| cct | ACC CAG | | RADNLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DKKDLTRHX₁₇ | |
| | CCT | | GKSFSDKKDLTRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDHLTTH | |
| | | | KCPECGKSFSRSDHLTTHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆SKKALT | |
| | | | GEKPYKCPECGKSFSSKKALTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆RNDA | |
| | | | QRTHTGEKPYKCPECGKSFSRND | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | ALTEHQRTHTGEKPYKCPECGKS | | DELVRHX₁₇X₁₈X₁₉HX₂₀X₂₁X₂₂X₂₃X₂₄X₂₅X₂₆X₂₇X₂₈X₂₉X₃₀ | |
| | | | FSRSDELVRHQRTHTGEKPTGKK | | | |
| | | | TS | | | |
| acagtgctg | ACA gtg | 1156 | LEPGEKPYKCPECGKSFSRADNL | 1705 | X₁X₂X₃X₄X₅X₆X₇X₈X₉CX₁₀X₁₁CX₁₂X₁₃X₁₄X₁₅X₁₆RADNLTEHX₁₇X₁₈X₁₉ | 2254 |
| cactggacc | CTG CAC | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆DKKDLTRHX₁₇X₁₈ | |
| cag | TGG ACC | | DKKDLTRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDHLTTHX₁₇ | |
| | CAG | | GKSFSRSDHLTTHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆SKKALTEH | |
| | | | KCPECGKSFSSKKALTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RNDALT | |
| | | | GEKPYKCPECGKSFSRNDALTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆RSDE | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆SP | |
| | | | ELVRHQRTHTGEKPYKCPECGKS | | ADLTRHX₁₇X₁₈X₁₉HX₂₀X₂₁X₂₂X₂₃X₂₄X₂₅X₂₆X₂₇X₂₈X₂₉X₃₀ | |
| | | | FSSPADLTRHQRTHTGEKPTGKK | | | |
| | | | TS | | | |
| ctgcactgg | CTG CAC | 1157 | LEPGEKPYKCPECGKSFSSPADL | 1706 | X₁X₂X₃X₄X₅X₆X₇X₈X₉CX₁₀X₁₁CX₁₂X₁₃X₁₄X₁₅X₁₆SPADLTRHX₁₇X₁₈X₁₉ | 2255 |
| acccagcct | TGG ACC | | TRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TKNSLTEHX₁₇X₁₈ | |
| aca | CAG CCT | | TKNSLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RADNLTEHX₁₇ | |
| | ACA | | GKSFSRADNLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DKKDLTRH | |
| | | | KCPECGKSFSDKKDLTRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDHLT | |
| | | | GEKPYKCPECGKSFSRSDHLTTH | | THX₁₇X₁₈X₁₉HX₂₀X₂₁X₂₂X₂₃X₂₄X₈X₉CX₁₀X₁₁CX₁₂X₁₃X₁₄X₁₅X₁₆SKKA | |
| | | | QRTHTGEKPYKCPECGKSFSSKK | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆RN | |
| | | | ALTEHQRTHTGEKPYKCPECGKS | | DALTEHX₁₇X₁₈X₁₉HX₂₀X₂₁X₂₂X₂₃X₂₄X₂₅X₂₆X₂₇X₂₈X₂₉X₃₀ | |
| | | | FSRNDALTEHQRTHTGEKPTGKK | | | |
| | | | TS | | | |
| cactggacc | CAC TGG | 1158 | LEPGEKPYKCPECGKSFSTSHSL | 1707 | X₁X₂X₃X₄X₅X₆X₇X₈X₉CX₁₀X₁₁CX₁₂X₁₃X₁₄X₁₅X₁₆TSHSLTEHX₁₇X₁₈X₁₉ | 2256 |
| cagcctaca | ACC CAG | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆SPADLTRHX₁₇X₁₈ | |
| cca | CCT ACA | | SPADLTRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TKNSLTEHX₁₇ | |
| | CCA | | GKSFSTKNSLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RADNLTEH | |
| | | | KCPECGKSFSRADNLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DKKDLT | |
| | | | GEKPYKCPECGKSFSDKKDLTRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDH | |
| | | | QRTHTGEKPYKCPECGKSFSRSD | | LTTHX₁₇X₁₈X₁₉HX₂₀X₂₁X₂₂X₂₃X₂₄X₈X₉CX₁₀X₁₁CX₁₂X₁₃X₁₄X₁₅X₁₆SK | |
| | | | HLTTHQRTHTGEKPYKCPECGKS | | KALTEHX₁₇X₁₈X₁₉HX₂₀X₂₁X₂₂X₂₃X₂₄X₂₅X₂₆X₂₇X₂₈X₂₉X₃₀ | |
| | | | FSSKKALTEHQRTHTGEKPTGKK | | | |
| | | | TS | | | |
| actacagtg | ACT ACA | 1159 | LEPGEKPYKCPECGKSFSDKKDL | 1708 | X₁X₂X₃X₄X₅X₆X₇X₈X₉CX₁₀X₁₁CX₁₂X₁₃X₁₄X₁₅X₁₆DKKDLTRHX₁₇X₁₈X₁₉ | 2257 |
| ctgcactgg | gtg CTG | | TRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDHLTTHX₁₇X₁₈ | |
| acc | CAC TGG | | RSDHLTTHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆SKKALTEHX₁₇ | |
| | ACC | | GKSFSSKKALTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RNDALTEH | |
| | | | KCPECGKSFSRNDALTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDELV | |
| | | | GEKPYKCPECGKSFSRSDELVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆SPAD | |
| | | | QRTHTGEKPYKCPECGKSFSSPA | | LTRHX₁₇X₁₈X₁₉HX₂₀X₂₁X₂₂X₂₃X₂₄X₈X₉CX₁₀X₁₁CX₁₂X₁₃X₁₄X₁₅X₁₆TH | |
| | | | DLTRHQRTHTGEKPYKCPECGKS | | LDLIRHX₁₇X₁₈X₁₉HX₂₀X₂₁X₂₂X₂₃X₂₄X₂₅X₂₆X₂₇X₂₈X₂₉X₃₀ | |
| | | | FSTHLDLIRHQRTHTGEKPTGKK | | | |
| | | | TS | | | |
| acatggccg | ACA TGG | 1160 | LEPGEKPYKCPECGKSFSRNDAL | 1709 | X₁X₂X₃X₄X₅X₆X₇X₈X₉CX₁₀X₁₁CX₁₂X₁₃X₁₄X₁₅X₁₆RNDALTEHX₁₇X₁₈X₁₉ | 2258 |
| actacagtg | CCG ACT | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDELVRHX₁₇X₁₈ | |
| ctg | ACA gtg | | RSDELVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆SPADLTRHX₁₇ | |
| | CTG | | GKSFSSPADLTRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆THLDLIRH | |
| | | | KCPECGKSFSTHLDLIRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RNDTLT | |
| | | | GEKPYKCPECGKSFSRNDTLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆RSDH | |
| | | | QRTHTGEKPYKCPECGKSFSRSD | | LTTHX₁₇X₁₈X₁₉HX₂₀X₂₁X₂₂X₂₃X₂₄X₈X₉CX₁₀X₁₁CX₁₂X₁₃X₁₄X₁₅X₁₆SP | |
| | | | HLTTHQRTHTGEKPYKCPECGKS | | ADLTRHX₁₇X₁₈X₁₉HX₂₀X₂₁X₂₂X₂₃X₂₄X₂₅X₂₆X₂₇X₂₈X₂₉X₃₀ | |
| | | | FSSPADLTRHQRTHTGEKPTGKK | | | |
| | | | TS | | | |
| tggacatgg | TGG ACA | 1161 | LEPGEKPYKCPECGKSFSRSDEL | 1710 | X₁X₂X₃X₄X₅X₆X₇X₈X₉CX₁₀X₁₁CX₁₂X₁₃X₁₄X₁₅X₁₆RSDELVRHX₁₇X₁₈X₁₉ | 2259 |
| ccgactaca | TGG CCG | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆SPADLTRHX₁₇X₁₈ | |
| gtg | ACT ACA | | SPADLTRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆THLDLIRHX₁₇ | |
| | GTG | | GKSFSTHLDLIRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RNDTLTEH | |
| | | | KCPECGKSFSRNDTLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDHLT | |
| | | | GEKPYKCPECGKSFSRSDHLTTH | | THX₁₇X₁₈X₁₉HX₂₀X₂₁X₂₂X₂₃X₂₄X₈X₉CX₁₀X₁₁CX₁₂X₁₃X₁₄X₁₅X₁₆SPAD | |
| | | | QRTHTGEKPYKCPECGKSFSSPA | | LTRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | DLTRHQRTHTGEKPYKCPECGKS | | DHLTTHX₁₇X₁₈X₁₉HX₂₀X₂₁X₂₂X₂₃X₂₄X₂₅X₂₆X₂₇X₂₈X₂₉X₃₀ | |
| | | | FSRSDHLTTHQRTHTGEKPTGKK | | | |
| | | | TS | | | |
| cagcctaca | CAG CCT | 1162 | LEPGEKPYKCPECGKSFSTTGNL | 1711 | X₁X₂X₃X₄X₅X₆X₇X₈X₉CX₁₀X₁₁CX₁₂X₁₃X₁₄X₁₅X₁₆TTGNLTVHX₁₇X₁₈X₁₉ | 2260 |
| ccaccctgg | ACA CCA | | TVHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDHLTTHX₁₇X₁₈ | |
| aat | CCC TGG | | RSDHLTTHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆SKKHLAEHX₁₇ | |
| | AAT | | GKSFSSKKHLAEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSHSLTEH | |
| | | | KCPECGKSFSTSHSLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆SPADLT | |
| | | | GEKPYKCPECGKSFSSPADLTRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆TKNS | |
| | | | QRTHTGEKPYKCPECGKSFSTKN | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆RA | |
| | | | SLTEHQRTHTGEKPYKCPECGKS | | DNLTEHX₁₇X₁₈X₁₉HX₂₀X₂₁X₂₂X₂₃X₂₄X₂₅X₂₆X₂₇X₂₈X₂₉X₃₀ | |
| | | | FSRADNLTEHQRTHTGEKPTGKK | | | |
| | | | TS | | | |
| acatggaca | ACA TGG | 1163 | LEPGEKPYKCPECGKSFSSPADL | 1712 | X₁X₂X₃X₄X₅X₆X₇X₈X₉CX₁₀X₁₁CX₁₂X₁₃X₁₄X₁₅X₁₆SPADLTRHX₁₇X₁₈X₁₉ | 2261 |
| tggccgact | ACA TGG | | TRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆THLDLIRHX₁₇X₁₈ | |
| aca | CCG ACT | | THLDLIRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RNDTLTEHX₁₇ | |
| | ACA | | GKSFSRNDTLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDHLTTH | |
| | | | KCPECGKSFSRSDHLTTHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆SPADLT | |
| | | | GEKPYKCPECGKSFSSPADLTRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDH | |
| | | | QRTHTGEKPYKCPECGKSFSRSD | | LTTHX₁₇X₁₈X₁₉HX₂₀X₂₁X₂₂X₂₃X₂₄X₈X₉CX₁₀X₁₁CX₁₂X₁₃X₁₄X₁₅X₁₆SP | |
| | | | HLTTHQRTHTGEKPYKCPECGKS | | ADLTRHX₁₇X₁₈X₁₉HX₂₀X₂₁X₂₂X₂₃X₂₄X₂₅X₂₆X₂₇X₂₈X₂₉X₃₀ | |
| | | | FSSPADLTRHQRTHTGEKPTGKK | | | |
| | | | TS | | | |
| tggacccag | TGG ACC | 1164 | LEPGEKPYKCPECGKSFSSKKHL | 1713 | X₁X₂X₃X₄X₅X₆X₇X₈X₉CX₁₀X₁₁CX₁₂X₁₃X₁₄X₁₅X₁₆SKKHLAEHX₁₇X₁₈X₁₉ | 2262 |
| cctacacca | CAG CCT | | AEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSHSLTEHX₁₇X₁₈ | |
| ccc | ACA CCA | | TSHSLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆SPADLTRHX₁₇ | |
| | CCC | | GKSFSSPADLTRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TKNSLTEH | |
| | | | KCPECGKSFSTKNSLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RADNLT | |
| | | | GEKPYKCPECGKSFSRADNLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆DKKD | |
| | | | QRTHTGEKPYKCPECGKSFSDKK | | LTRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | DLTRHQRTHTGEKPYKCPECGKS | | DHLTTHX₁₇X₁₈X₁₉HX₂₀X₂₁X₂₂X₂₃X₂₄X₂₅X₂₆X₂₇X₂₈X₂₉X₃₀ | |
| | | | FSRSDHLTTHQRTHTGEKPTGKK | | | |
| | | | TS | | | |
| acccagcct | ACC CAG | 1165 | LEPGEKPYKCPECGKSFSRSDHL | 1714 | X₁X₂X₃X₄X₅X₆X₇X₈X₉CX₁₀X₁₁CX₁₂X₁₃X₁₄X₁₅X₁₆RSDHLTTHX₁₇X₁₈X₁₉ | 2263 |
| acaccaccc | CCT ACA | | TTHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆SKKHLAEHX₁₇X₁₈ | |
| tgg | CCA CCC | | SKKHLAEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSHSLTEHX₁₇ | |
| | TGG | | GKSFSTSHSLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆SPADLTRH | |
| | | | KCPECGKSFSSPADLTRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TKNSLT | |
| | | | GEKPYKCPECGKSFSTKNSLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆RADN | |
| | | | QRTHTGEKPYKCPECGKSFSRAD | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆DK | |
| | | | NLTEHQRTHTGEKPYKCPECGKS | | KDLTRHX₁₇X₁₈X₁₉HX₂₀X₂₁X₂₂X₂₃X₂₄X₂₅X₂₆X₂₇X₂₈X₂₉X₃₀ | |
| | | | FSDKKDLTRHQRTHTGEKPTGKK | | | |
| | | | TS | | | |
| ccgactaca | CCG ACT | 1166 | LEPGEKPYKCPECGKSFSRSDHL | 1715 | X₁X₂X₃X₄X₅X₆X₇X₈X₉CX₁₀X₁₁CX₁₂X₁₃X₁₄X₁₅X₁₆RSDHLTTHX₁₇X₁₈X₁₉ | 2264 |
| gtgctgcac | ACA gtg | | TTHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆SKKALTEHX₁₇X₁₈ | |
| tgg | CTG CAC | | SKKALTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RNDALTEHX₁₇ | |
| | TGG | | GKSFSRNDALTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDELVRH | |
| | | | KCPECGKSFSRSDELVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆SPADLT | |
| | | | GEKPYKCPECGKSFSSPADLTRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆THLD | |
| | | | QRTHTGEKPYKCPECGKSFSTHL | | LIRHX₁₇X₁₈X₁₉HX₂₀X₂₁X₂₂X₂₃X₂₄X₈X₉CX₁₀X₁₁CX₁₂X₁₃X₁₄X₁₅X₁₆RN | |
| | | | DLIRHQRTHTGEKPYKCPECGKS | | DTLTEHX₁₇X₁₈X₁₉HX₂₀X₂₁X₂₂X₂₃X₂₄X₂₅X₂₆X₂₇X₂₈X₂₉X₃₀ | |
| | | | FSRNDTLTEHQRTHTGEKPTGKK | | | |
| | | | TS | | | |
| tggccgact | TGG CCG | 1167 | LEPGEKPYKCPECGKSFSSKKAL | 1716 | X₁X₂X₃X₄X₅X₆X₇X₈X₉CX₁₀X₁₁CX₁₂X₁₃X₁₄X₁₅X₁₆SKKALTEHX₁₇X₁₈X₁₉ | 2265 |
| acagtgctg | ACT ACA | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RNDALTEHX₁₇X₁₈ | |
| cac | gtg CTG | | RNDALTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDELVRHX₁₇ | |
| | CAC | | GKSFSRSDELVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆SPADLTRH | |
| | | | KCPECGKSFSSPADLTRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆THLDLI | |
| | | | GEKPYKCPECGKSFSTHLDLIRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RNDT | |
| | | | QRTHTGEKPYKCPECGKSFSRND | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | TLTEHQRTHTGEKPYKCPECGKS | | DHLTTHX₁₇X₁₈X₁₉HX₂₀X₂₁X₂₂X₂₃X₂₄X₂₅X₂₆X₂₇X₂₈X₂₉X₃₀ | |
| | | | FSRSDHLTTHQRTHTGEKPTGKK | | | |
| | | | TS | | | |
| agtgctgca | AGT GCT | 1168 | LEPGEKPYKCPECGKSFSDCRDL | 1717 | X₁X₂X₃X₄X₅X₆X₇X₈X₉CX₁₀X₁₁CX₁₂X₁₃X₁₄X₁₅X₁₆DCRDLARHX₁₇X₁₈X₁₉ | 2266 |
| ctggaccca | GCA CTG | | ARHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSHSLTEHX₁₇X₁₈ | |
| gcc | GAC CCA | | TSHSLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DPGNLVRHX₁₇ | |
| | GCC | | GKSFSDPGNLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RNDALTEH | |
| | | | KCPECGKSFSRNDALTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QSGDLR | |
| | | | GEKPYKCPECGKSFSQSGDLRRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆TSGE | |
| | | | QRTHTGEKPYKCPECGKSFSTSG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆HR | |
| | | | ELVRHQRTHTGEKPYKCPECGKS | | TTLTNHX₁₇X₁₈X₁₉HX₂₀X₂₁X₂₂X₂₃X₂₄X₂₅X₂₆X₂₇X₂₈X₂₉X₃₀ | |
| | | | FSHRTTLTNHQRTHTGEKPTGKK | | | |
| | | | TS | | | |
| ctacaccac | CTA CAC | 1169 | LEPGEKPYKCPECGKSFSQAGHL | 1718 | X₁X₂X₃X₄X₅X₆X₇X₈X₉CX₁₀X₁₁CX₁₂X₁₃X₁₄X₁₅X₁₆QAGHLASHX₁₇X₁₈X₁₉ | 2267 |
| cctggaatt | CAC CCT | | ASHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆HKNALQNHX₁₇X₁₈ | |
| tga | GGA ATT | | HKNALQNHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QRAHLERHX₁₇ | |
| | TGA | | GKSFSQRAHLERHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TKNSLTEH | |
| | | | KCPECGKSFSTKNSLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆SKKALT | |
| | | | GEKPYKCPECGKSFSSKKALTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆SKKA | |
| | | | QRTHTGEKPYKCPECGKSFSSKK | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆QN | |
| | | | ALTEHQRTHTGEKPYKCPECGKS | | STLTEHX₁₇X₁₈X₁₉HX₂₀X₂₁X₂₂X₂₃X₂₄X₂₅X₂₆X₂₇X₂₈X₂₉X₃₀ | |
| | | | FSQNSTLTEHQRTHTGEKPTGKK | | | |
| | | | TS | | | |
| agcctacac | AGC CTA | 1170 | LEPGEKPYKCPECGKSFSHKNAL | 1719 | X₁X₂X₃X₄X₅X₆X₇X₈X₉CX₁₀X₁₁CX₁₂X₁₃X₁₄X₁₅X₁₆HKNALQNHX₁₇X₁₈X₁₉ | 2268 |
| caccctgga | CAC CAC | | QNHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QRAHLERHX₁₇X₁₈ | |
| att | CCT GGA | | QRAHLERHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TKNSLTEHX₁₇ | |
| | ATT | | GKSFSTKNSLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆SKKALTEH | |
| | | | KCPECGKSFSSKKALTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆SKKALT | |
| | | | GEKPYKCPECGKSFSSKKALTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆QNST | |
| | | | QRTHTGEKPYKCPECGKSFSQNS | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆ER | |
| | | | TLTEHQRTHTGEKPYKCPECGKS | | SHLREHX₁₇X₁₈X₁₉HX₂₀X₂₁X₂₂X₂₃X₂₄X₂₅X₂₆X₂₇X₂₈X₂₉X₃₀ | |
| | | | FSERSHLREHQRTHTGEKPTGKK | | | |
| | | | TS | | | |
| caccaccct | CAC CAC | 1171 | LEPGEKPYKCPECGKSFSQSSNL | 1720 | X₁X₂X₃X₄X₅X₆X₇X₈X₉CX₁₀X₁₁CX₁₂X₁₃X₁₄X₁₅X₁₆QSSNLVRHX₁₇X₁₈X₁₉ | 2269 |
| ggaatttga | CCT GGA | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QAGHLASHX₁₇X₁₈ | |
| gaa | ATT TGA | | QAGHLASHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆HKNALQNHX₁₇ | |
| | GAA | | GKSFSHKNALQNHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QRAHLERH | |
| | | | KCPECGKSFSQRAHLERHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TKNSLT | |
| | | | GEKPYKCPECGKSFSTKNSLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆SKKA | |
| | | | QRTHTGEKPYKCPECGKSFSSKK | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆SK | |
| | | | ALTEHQRTHTGEKPYKCPECGKS | | KALTEHX₁₇X₁₃X₁₉HX₂₀X₂₁X₂₂X₂₃X₂₄X₂₅X₂₆X₂₇X₂₈X₂₉X₃₀ | |
| | | | FSSKKALTEHQRTHTGEKPTGKK | | | |
| | | | TS | | | |
| ggacccagc | GGA CCC | 1172 | LEPGEKPYKCPECGKSFSTKNSL | 1721 | X₁X₂X₃X₄X₅X₆X₇X₈X₉CX₁₀X₁₁CX₁₂X₁₃X₁₄X₁₅X₁₆TKNSLTEHX₁₇X₁₈X₁₉ | 2270 |
| ctacaccac | AGC CTA | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆SKKALTEHX₁₇X₁₈ | |
| cct | CAC CAC | | SKKALTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆SKKALTEHX₁₇ | |
| | CCT | | GKSFSSKKALTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QNSTLTEH | |
| | | | KCPECGKSFSQNSTLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆ERSHLR | |
| | | | GEKPYKCPECGKSFSERSHLREH | | EHX₁₇X₁₈X₁₉HX₂₀X₂₁X₂₂X₂₃X₂₄X₈X₉CX₁₀X₁₁CX₁₂X₁₃X₁₄X₁₅X₁₆SKKH | |
| | | | QRTHTGEKPYKCPECGKSFSSKK | | LAEHX₁₇X₁₈X₁₉HX₂₀X₂₁X₂₂X₂₃X₂₄X₈X₉CX₁₀X₁₁CX₁₂X₁₃X₁₄X₁₅X₁₆QR | |
| | | | HLAEHQRTHTGEKPYKCPECGKS | | AHLERHX₁₇X₁₈X₁₉HX₂₀X₂₁X₂₂X₂₃X₂₄X₂₅X₂₆X₂₇X₂₈X₂₉X₃₀ | |
| | | | FSQRAHLERHQRTHTGEKPTGKK | | | |
| | | | TS | | | |
| cccagccta | CCC AGC | 1173 | LEPGEKPYKCPECGKSFSQRAHL | 1722 | X₁X₂X₃X₄X₅X₆X₇X₈X₉CX₁₀X₁₁CX₁₂X₁₃X₁₄X₁₅X₁₆QRAHLERHX₁₇X₁₈X₁₉ | 2271 |
| caccaccct | CTA CAC | | ERHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TKNSLTEHX₁₇X₁₈ | |
| gga | CAC CCT | | TKNSLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆SKKALTEHX₁₇ | |
| | GGA | | GKSFSSKKALTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆SKKALTEH | |
| | | | KCPECGKSFSSKKALTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QNSTLT | |
| | | | GEKPYKCPECGKSFSQNSTLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆ERSH | |
| | | | QRTHTGEKPYKCPECGKSFSERS | | LREHX₁₇X₁₈X₁₉HX₂₀X₂₁X₂₂X₂₃X₂₄X₈X₉CX₁₀X₁₁CX₁₂X₁₃X₁₄X₁₅X₁₆SK | |
| | | | HLREHQRTHTGEKPYKCPECGKS | | KHLAEHX₁₇X₁₈X₁₉HX₂₀X₂₁X₂₂X₂₃X₂₄X₂₅X₂₆X₂₇X₂₈X₂₉X₃₀ | |
| | | | FSSKKHLAEHQRTHTGEKPTGKK | | | |
| | | | TS | | | |
| actggaccc | ACT GGA | 1174 | LEPGEKPYKCPECGKSFSSKKAL | 1723 | X₁X₂X₃X₄X₅X₆X₇X₈X₉CX₁₀X₁₁CX₁₂X₁₃X₁₄X₁₅X₁₆SKKALTEHX₁₇X₁₈X₁₉ | 2272 |
| agcctacac | CCC AGC | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆SKKALTEHX₁₇X₁₈ | |
| cac | CTA CAC | | SKKALTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QNSTLTEHX₁₇ | |
| | CAC | | GKSFSQNSTLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆ERSHLREH | |
| | | | KCPECGKSFSERSHLREHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆SKKHLA | |
| | | | GEKPYKCPECGKSFSSKKHLAEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆QRAH | |
| | | | QRTHTGEKPYKCPECGKSFSQRA | | LERHX₁₇X₁₈X₁₉HX₂₀X₂₁X₂₂X₂₃X₂₄X₈X₉CX₁₀X₁₁CX₁₂X₁₃X₁₄X₁₅X₁₆TH | |
| | | | HLERHQRTHTGEKPYKCPECGKS | | LDLIRHX₁₇X₁₈X₁₉HX₂₀X₂₁X₂₂X₂₃X₂₄X₂₅X₂₆X₂₇X₂₈X₂₉X₃₀ | |
| | | | FSTHLDLIRHQRTHTGEKPTGKK | | | |
| | | | TS | | | |
| tggtaggtg | TGG TAG | 1175 | LEPGEKPYKCPECGKSFSRSDEL | 1724 | X₁X₂X₃X₄X₅X₆X₇X₈X₉CX₁₀X₁₁CX₁₂X₁₃X₁₄X₁₅X₁₆RSDELVRHX₁₇X₁₈X₁₉ | 2273 |
| ggggcagat | GTG GGG | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSGNLVRHX₁₇X₁₈ | |
| gtg | GCA GAT | | TSGNLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QSGDLRRHX₁₇ | |
| | GTG | | GKSFSQSGDLRRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDKLVRH | |
| | | | KCPECGKSFSRSDKLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDELV | |
| | | | GEKPYKCPECGKSFSRSDELVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆REDN | |
| | | | QRTHTGEKPYKCPECGKSFSRED | | LHTHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | NLHTHQRTHTGEKPYKCPECGKS | | DHLTTHX₁₇X₁₈X₁₉HX₂₀X₂₁X₂₂X₂₃X₂₄X₂₅X₂₆X₂₇X₂₈X₂₉X₃₀ | |
| | | | FSRSDHLTTHQRTHTGEKPTGKK | | | |
| | | | TS | | | |
| taggtgggg | TAG GTG | 1176 | LEPGEKPYKCPECGKSFSSKKHL | 1725 | X₁X₂X₃X₄X₅X₆X₇X₈X₉CX₁₀X₁₁CX₁₂X₁₃X₁₄X₁₅X₁₆SKKHLAEHX₁₇X₁₈X₁₉ | 2274 |
| gcagatgtg | GGG GCA | | AEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDELVRHX₁₇X₁₈ | |
| ccc | GAT gtg | | RSDELVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSGNLVRHX₁₇ | |
| | CCC | | GKSFSTSGNLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QSGDLRRH | |
| | | | KCPECGKSFSQSGDLRRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDKLV | |
| | | | GEKPYKCPECGKSFSRSDKLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDE | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RE | |
| | | | ELVRHQRTHTGEKPYKCPECGKS | | DNLHTHX₁₇X₁₈X₁₉HX₂₀X₂₁X₂₂X₂₃X₂₄X₂₅X₂₆X₂₇X₂₈X₂₉X₃₀ | |
| | | | FSREDNLHTHQRTHTGEKPTGKK | | | |
| | | | TS | | | |
| gatgggcaa | GAT ggg | 1177 | LEPGEKPYKCPECGKSFSRSDKL | 1726 | X₁X₂X₃X₄X₅X₆X₇X₈X₉CX₁₀X₁₁CX₁₂X₁₃X₁₄X₁₅X₁₆RSDKLVRHX₁₇X₁₈X₁₉ | 2275 |
| tggtaggtg | CAA TGG | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDELVRHX₁₇X₁₈ | |
| ggg | TAG GTG | | RSDELVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆REDNLHTHX₁₇ | |
| | GGG | | GKSFSREDNLHTHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDHLTTH | |
| | | | KCPECGKSFSRSDHLTTHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QSGNLT | |
| | | | GEKPYKCPECGKSFSQSGNLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆RSDK | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | KLVRHQRTHTGEKPYKCPECGKS | | GNLVRHX₁₇X₁₈X₁₉HX₂₀X₂₁X₂₂X₂₃X₂₄X₂₅X₂₆X₂₇X₂₈X₂₉X₃₀ | |
| | | | FSTSGNLVRHQRTHTGEKPTGKK | | | |
| | | | TS | | | |
| gggcaatgg | ggg CAA | 1178 | LEPGEKPYKCPECGKSFSQSGDL | 1727 | X₁X₂X₃X₄X₅X₆X₇X₈X₉CX₁₀X₁₁CX₁₂X₁₃X₁₄X₁₅X₁₆QSGDLRRHX₁₇X₁₈X₁₉ | 2276 |
| taggtgggg | TGG TAG | | RRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDKLVRHX₁₇X₁₈ | |
| gca | GTG GGG | | RSDKLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDELVRHX₁₇ | |
| | GCA | | GKSFSRSDELVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆REDNLHTH | |
| | | | KCPECGKSFSREDNLHTHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDHLT | |
| | | | GEKPYKCPECGKSFSRSDHLTTH | | THX₁₇X₁₈X₁₉HX₂₀X₂₁X₂₂X₂₃X₂₄X₈X₉CX₁₀X₁₁CX₁₂X₁₃X₁₄X₁₅X₁₆QSGN | |
| | | | QRTHTGEKPYKCPECGKSFSQSG | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | NLTEHQRTHTGEKPYKCPECGKS | | DKLVRHX₁₇X₁₈X₁₉HX₂₀X₂₁X₂₂X₂₃X₂₄X₂₅X₂₆X₂₇X₂₈X₂₉X₃₀ | |
| | | | FSRSDKLVRHQRTHTGEKPTGKK | | | |
| | | | TS | | | |
| caatggtag | CAA TGG | 1179 | LEPGEKPYKCPECGKSFSTSGNL | 1728 | X₁X₂X₃X₄X₅X₆X₇X₈X₉CX₁₀X₁₁CX₁₂X₁₃X₁₄X₁₅X₁₆TSGNLVRHX₁₇X₁₈X₁₉ | 2277 |
| gtgggggca | TAG GTG | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QSGDLRRHX₁₇X₁₈ | |
| gat | GGG GCA | | QSGDLRRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDKLVRHX₁₇ | |
| | GAT | | GKSFSRSDKLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDELVRH | |
| | | | KCPECGKSFSRSDELVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆REDNLH | |
| | | | GEKPYKCPECGKSFSREDNLHTH | | THX₁₇X₁₈X₁₉HX₂₀X₂₁X₂₂X₂₃X₂₄X₈X₉CX₁₀X₁₁CX₁₂X₁₃X₁₄X₁₅X₁₆RSDH | |
| | | | QRTHTGEKPYKCPECGKSFSRSD | | LTTHX₁₇X₁₈X₁₉HX₂₀X₂₁X₂₂X₂₃X₂₄X₈X₉CX₁₀X₁₁CX₁₂X₁₃X₁₄X₁₅X₁₆QS | |
| | | | HLTTHQRTHTGEKPYKCPECGKS | | GNLTEHX₁₇X₁₈X₁₉HX₂₀X₂₁X₂₂X₂₃X₂₄X₂₅X₂₆X₂₇X₂₈X₂₉X₃₀ | |
| | | | FSQSGNLTEHQRTHTGEKPTGKK | | | |
| | | | TS | | | |
| gtgggggca | GTG GGG | 1180 | LEPGEKPYKCPECGKSFSRSDHL | 1729 | X₁X₂X₃X₄X₅X₆X₇X₈X₉CX₁₀X₁₁CX₁₂X₁₃X₁₄X₁₅X₁₆RSDHLTNHX₁₇X₁₈X₁₉ | 2278 |
| gatgtgccc | GCA GAT | | TNHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆SKKHLAEHX₁₇X₁₈ | |
| agg | gtg CCC | | SKKHLAEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDELVRHX₁₇ | |
| | AGG | | GKSFSRSDELVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSGNLVRH | |
| | | | KCPECGKSFSTSGNLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QSGDLR | |
| | | | GEKPYKCPECGKSFSQSGDLRRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDK | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | KLVRHQRTHTGEKPYKCPECGKS | | DELVRHX₁₇X₁₈X₁₉HX₂₀X₂₁X₂₂X₂₃X₂₄X₂₅X₂₆X₂₇X₂₈X₂₉X₃₀ | |
| | | | FSRSDELVRHQRTHTGEKPTGKK | | | |
| | | | TS | | | |
| gacgatggg | GAC GAT | 1181 | LEPGEKPYKCPECGKSFSRSDEL | 1730 | X₁X₂X₃X₄X₅X₆X₇X₈X₉CX₁₀X₁₁CX₁₂X₁₃X₁₄X₁₅X₁₆RSDELVRHX₁₇X₁₈X₁₉ | 2279 |
| caatggtag | ggg CAA | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆REDNLHTHX₁₇X₁₈ | |
| gtg | TGG TAG | | REDNLHTHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDHLTTHX₁₇ | |
| | GTG | | GKSFSRSDHLTTHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QSGNLTEH | |
| | | | KCPECGKSFSQSGNLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDKLV | |
| | | | GEKPYKCPECGKSFSRSDKLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆TSGN | |
| | | | QRTHTGEKPYKCPECGKSFSTSG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆DP | |
| | | | NLVRHQRTHTGEKPYKCPECGKS | | GNLVRHX₁₇X₁₈X₁₉HX₂₀X₂₁X₂₂X₂₃X₂₄X₂₅X₂₆X₂₇X₂₈X₂₉X₃₀ | |
| | | | FSDPGNLVRHQRTHTGEKPTGKK | | | |
| | | | TS | | | |
| gttgacgat | GTT GAC | 1182 | LEPGEKPYKCPECGKSFSREDNL | 1731 | X₁X₂X₃X₄X₅X₆X₇X₈X₉CX₁₀X₁₁CX₁₂X₁₃X₁₄X₁₅X₁₆REDNLHTHX₁₇X₁₈X₁₉ | 2280 |
| gggcaatgg | GAT ggg | | HTHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDHLTTHX₁₇X₁₈ | |
| tag | CAA TGG | | RSDHLTTHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QSGNLTEHX₁₇ | |
| | TAG | | GKSFSQSGNLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDKLVRH | |
| | | | KCPECGKSFSRSDKLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSGNLV | |
| | | | GEKPYKCPECGKSFSTSGNLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆DPGN | |
| | | | QRTHTGEKPYKCPECGKSFSDPG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | NLVRHQRTHTGEKPYKCPECGKS | | GSLVRHX₁₇X₁₈X₁₉HX₂₀X₂₁X₂₂X₂₃X₂₄X₂₅X₂₆X₂₇X₂₈X₂₉X₃₀ | |
| | | | FSTSGSLVRHQRTHTGEKPTGKK | | | |
| | | | TS | | | |
| gcaggtgtt | GCA GGT | 1183 | LEPGEKPYKCPECGKSFSQSGNL | 1732 | X₁X₂X₃X₄X₅X₆X₇X₈X₉CX₁₀X₁₁CX₁₂X₁₃X₁₄X₁₅X₁₆QSGNLTEHX₁₇X₁₈X₁₉ | 2281 |
| gacgatggg | GTT GAC | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDKLVRHX₁₇X₁₈ | |
| caa | GAT ggg | | RSDKLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSGNLVRHX₁₇ | |
| | CAA | | GKSFSTSGNLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DPGNLVRH | |
| | | | KCPECGKSFSDPGNLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSGSLV | |
| | | | GEKPYKCPECGKSFSTSGSLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆TSGH | |
| | | | QRTHTGEKPYKCPECGKSFSTSG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆QS | |
| | | | HLVRHQRTHTGEKPYKCPECGKS | | GDLRRHX₁₇X₁₈X₁₉HX₂₀X₂₁X₂₂X₂₃X₂₄X₂₅X₂₆X₂₇X₂₈X₂₉X₃₀ | |
| | | | FSQSGDLRRHQRTHTGEKPTGKK | | | |
| | | | TS | | | |
| ggtgttgac | GGT GTT | 1184 | LEPGEKPYKCPECGKSFSRSDHL | 1733 | X₁X₂X₃X₄X₅X₆X₇X₈X₉CX₁₀X₁₁CX₁₂X₁₃X₁₄X₁₅X₁₆RSDHLTTHX₁₇X₁₈X₁₉ | 2282 |
| gatgggcaa | GAC GAT | | TTHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QSGNLTEHX₁₇X₁₈ | |
| tgg | ggg CAA | | QSGNLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDKLVRHX₁₇ | |
| | TGG | | GKSFSRSDKLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSGNLVRH | |
| | | | KCPECGKSFSTSGNLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DPGNLV | |
| | | | GEKPYKCPECGKSFSDPGNLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆TSGS | |
| | | | QRTHTGEKPYKCPECGKSFSTSG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | SLVRHQRTHTGEKPYKCPECGKS | | GHLVRHX₁₇X₁₈X₁₉HX₂₀X₂₁X₂₂X₂₃X₂₄X₂₅X₂₆X₂₇X₂₈X₂₉X₃₀ | |
| | | | FSTSGHLVRHQRTHTGEKPTGKK | | | |
| | | | TS | | | |
| gcaatggta | GCA ATG | 1185 | LEPGEKPYKCPECGKSFSQLAHL | 1734 | X₁X₂X₃X₄X₅X₆X₇X₈X₉CX₁₀X₁₁CX₁₂X₁₃X₁₄X₁₅X₁₆QLAHLRAHX₁₇X₁₈X₁₉ | 2283 |
| ggtgggggc | GTA GGT | | RAHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆DPGHLVRHX₁₇X₁₈ | |
| aga | GGG GGC | | DPGHLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDKLVRHX₁₇ | |
| | AGA | | GKSFSRSDKLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSGHLVRH | |
| | | | KCPECGKSFSTSGHLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QSSSLV | |
| | | | GEKPYKCPECGKSFSQSSSLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RRDE | |
| | | | QRTHTGEKPYKCPECGKSFSRRD | | LNVHX₁₇X₁₈X₁₉HX₂₀X₂₁X₂₂X₂₃X₂₄X₈X₉CX₁₀X₁₁CX₁₂X₁₃X₁₄X₁₅X₁₆QS | |
| | | | ELNVHQRTHTGEKPYKCPECGKS | | GDLRRHX₁₇X₁₈X₁₉HX₂₀X₂₁X₂₂X₂₃X₂₄X₂₅X₂₆X₂₇X₂₈X₂₉X₃₀ | |
| | | | FSQSGDLRRHQRTHTGEKPTGKK | | | |
| | | | TS | | | |
| tgggcaatg | TGG GCA | 1186 | LEPGEKPYKCPECGKSFSDPGHL | 1735 | X₁X₂X₃X₄X₅X₆X₇X₈X₉CX₁₀X₁₁CX₁₂X₁₃X₁₄X₁₅X₁₆DPGHLVRHX₁₇X₁₈X₁₉ | 2284 |
| gtaggtggg | ATG GTA | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDKLVRHX₁₇X₁₈ | |
| ggc | GGT GGG | | RSDKLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSGHLVRHX₁₇ | |
| | GGC | | GKSFSTSGHLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QSSSLVRH | |
| | | | KCPECGKSFSQSSSLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RRDELN | |
| | | | GEKPYKCPECGKSFSRRDELNVH | | VHX₁₇X₁₈X₁₉HX₂₀X₂₁X₂₂X₂₃X₂₄X₈X₉CX₁₀X₁₁CX₁₂X₁₃X₁₄X₁₅X₁₆4SGD | |
| | | | QRTHTGEKPYKCPECGKSFSQSG | | LRRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | DLRRHQRTHTGEKPYKCPECGKS | | DHLTTHX₁₇X₁₈X₁₉HX₂₀X₂₁X₂₂X₂₃X₂₄X₂₅X₂₆X₂₇X₂₈X₂₉X₃₀ | |
| | | | FSRSDHLTTHQRTHTGEKPTGKK | | | |
| | | | TS | | | |
| tgacgatgg | TGA CGA | 1187 | LEPGEKPYKCPECGKSFSTSGHL | 1736 | X₁X₂X₃X₄X₅X₆X₇X₈X₉CX₁₀X₁₁CX₁₂X₁₃X₁₄X₁₅X₁₆TSGHLVRHX₁₇X₁₈X₁₉ | |
| gcaatggta | TGG GCA | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QSSSLVRHX₁₇X₁₈ | 2285 |
| ggt | ATG GTA | | QSSSLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RRDELNVHX₁₇ | |
| | GGT | | GKSFSRRDELNVHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QSGDLRRH | |
| | | | KCPECGKSFSQSGDLRRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDHLT | |
| | | | GEKPYKCPECGKSFSRSDHLTTH | | THX₁₇X₁₈X₁₉HX₂₀X₂₁X₂₂X₂₃X₂₄X₈X₉CX₁₀X₁₁CX₁₂X₁₃X₁₄X₁₅X₁₆QSGH | |
| | | | QRTHTGEKPYKCPECGKSFSQSG | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆QA | |
| | | | HLTEHQRTHTGEKPYKCPECGKS | | GHLASHX₁₇X₁₈X₁₉HX₂₀X₂₁X₂₂X₂₃X₂₄X₂₅X₂₆X₂₇X₂₈X₂₉X₃₀ | |
| | | | FSQAGHLASHQRTHTGEKPTGKK | | | |
| | | | TS | | | |
| cgatgggca | CGA TGG | 1188 | LEPGEKPYKCPECGKSFSRSDKL | 1737 | X₁X₂X₃X₄X₅X₆X₇X₈X₉CX₁₀X₁₁CX₁₂X₁₃X₁₄X₁₅X₁₆RSDKLVRHX₁₇X₁₈X₁₉ | 2286 |
| atggtaggt | GCA ATG | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSGHLVRHX₁₇X₁₈ | |
| ggg | GTA GGT | | TSGHLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QSSSLVRHX₁₇ | |
| | GGG | | GKSFSQSSSLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RRDELNVH | |
| | | | KCPECGKSFSRRDELNVHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QSGDLR | |
| | | | GEKPYKCPECGKSFSQSGDLRRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDH | |
| | | | QRTHTGEKPYKCPECGKSFSRSD | | LTTHX₁₇X₁₈X₁₉HX₂₀X₂₁X₂₂X₂₃X₂₄X₈X₉CX₁₀X₁₁CX₁₂X₁₃X₁₄X₁₅X₁₆QS | |
| | | | HLTTHQRTHTGEKPYKCPECGKS | | GHLTEHX₁₇X₁₈X₁₉HX₂₀X₂₁X₂₂X₂₃X₂₄X₂₅X₂₆X₂₇X₂₈X₂₉X₃₀ | |
| | | | FSQSGHLTEHQRTHTGEKPTGKK | | | |
| | | | TS | | | |
| ggcaatggt | GGC AAT | 1189 | LEPGEKPYKCPECGKSFSRADNL | 1738 | X₁X₂X₃X₄X₅X₆X₇X₈X₉CX₁₀X₁₁CX₁₂X₁₃X₁₄X₁₅X₁₆RADNLTEHX₁₇X₁₈X₁₉ | 2287 |
| aggtggggg | GGT AGG | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDKLVRHX₁₇X₁₈ | |
| cag | TGG ggg | | RSDKLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDHLTTHX₁₇ | |
| | CAG | | GKSFSRSDHLTTHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDHLTNH | |
| | | | KCPECGKSFSRSDHLTNHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSGHLV | |
| | | | GEKPYKCPECGKSFSTSGHLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆TTGN | |
| | | | QRTHTGEKPYKCPECGKSFSTTG | | LTVHX₁₇X₁₈X₁₉HX₂₀X₂₁X₂₂X₂₃X₂₄X₈X₉CX₁₀X₁₁CX₁₂X₁₃X₁₄X₁₅X₁₆DP | |
| | | | NLTVHQRTHTGEKPYKCPECGKS | | GHLVRHX₁₇X₁₈X₁₉HX₂₀X₂₁X₂₂X₂₃X₂₄X₂₅X₂₆X₂₇X₂₈X₂₉X₃₀ | |
| | | | FSDPGHLVRHQRTHTGEKPTGKK | | | |
| | | | TS | | | |
| atgggcaat | ATG GGC | 1190 | LEPGEKPYKCPECGKSFSRSDKL | 1739 | XX₂X₃X₄X₅X₆X₇X₈X₉CX₁₀X₁₁CX₁₂X₁₃X₁₄X₁₅X₁₆RSDKLVRHX₁₇X₁₈X₁₉ | 2288 |
| ggtaggtgg | AAT GGT | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDHLTTHX₁₇X₁₈ | |
| ggg | AGG TGG | | RSDHLTTHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDHLTNHX₁₇ | |
| | GGG | | GKSFSRSDHLTNHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSGHLVRH | |
| | | | KCPECGKSFSTSGHLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TTGNLT | |
| | | | GEKPYKCPECGKSFSTTGNLTVH | | VHX₁₇X₁₈X₁₉HX₂₀X₂₁X₂₂X₂₃X₂₄X₈X₉CX₁₀X₁₁CX₁₂X₁₃X₁₄X₁₅X₁₆DPGH | |
| | | | QRTHTGEKPYKCPECGKSFSDPG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RR | |
| | | | HLVRHQRTHTGEKPYKCPECGKS | | DELNVHX₁₇X₁₈X₁₉HX₂₀X₂₁X₂₂X₂₃X₂₄X₂₅X₂₆X₂₇X₂₈X₂₉X₃₀ | |
| | | | FSRRDELNVHQRTHTGEKPTGKK | | | |
| | | | TS | | | |
| aatggtagg | AAT GGT | 1191 | LEPGEKPYKCPECGKSFSRRDEL | 1740 | X₁X₂X₃X₄X₅X₆X₇X₈X₉CX₁₀X₁₁CX₁₂X₁₃X₁₄X₁₅X₁₆RRDELNVHX₁₇X₁₈X₁₉ | 2289 |
| tgggggcag | AGG TGG | | NVHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RADNLTEHX₁₇X₁₈ | |
| atg | ggg CAG | | RADNLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDKLVRHX₁₇ | |
| | ATG | | GKSFSRSDKLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDHLTTH | |
| | | | KCPECGKSFSRSDHLTTHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDHLT | |
| | | | GEKPYKCPECGKSFSRSDHLTNH | | NHX₁₇X₁₈X₁₉HX₂₀X₂₁X₂₂X₂₃X₂₄X₈X₉CX₁₀X₁₁CX₁₂X₁₃X₁₄X₁₅X₁₆TSGH | |
| | | | QRTHTGEKPYKCPECGKSFSTSG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆TT | |
| | | | HLVRHQRTHTGEKPYKCPECGKS | | GNLTVHX₁₇X₁₈X₁₉HX₂₀X₂₁X₂₂X₂₃X₂₄X₂₅X₂₆X₂₇X₂₈X₂₉X₃₀ | |
| | | | FSTTGNLTVHQRTHTGEKPTGKK | | | |
| | | | TS | | | |
| acgatgggc | ACG ATG | 1192 | LEPGEKPYKCPECGKSFSRSDHL | 1741 | X₁X₂X₃X₄X₅X₆X₇X₈X₉CX₁₀X₁₁CX₁₂X₁₃X₁₄X₁₅X₁₆RSDHLTTHX₁₇X₁₈X₁₉ | 2290 |
| aatggtagg | GGC AAT | | TTHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDHLTNHX₁₇X₁₈ | |
| tgg | GGT AGG | | RSDHLTNHQRTHTGEKPYKCPEC | | X₁₉HX₂₀X₂₁X₂₂X₂₃X₂₄X₈X₉CKX₁₀X₁₁CX₁₂X₁₃X₁₄X₁₅X₁₆TSGHLVRHX₁₇ | |
| | TGG | | GKSFSTSGHLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TTGNLTVH | |
| | | | KCPECGKSFSTTGNLTVHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DPGHLV | |
| | | | GEKPYKCPECGKSFSDPGHLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RRDE | |
| | | | QRTHTGEKPYKCPECGKSFSRRD | | LNVHX₁₇X₁₈X₁₉HX₂₀X₂₁X₂₂X₂₃X₂₄X₈X₉CX₁₀X₁₁CX₁₂X₁₃X₁₄X₁₅X₁₆RT | |
| | | | ELNVHQRTHTGEKPYKCPECGKS | | DTLRDHX₁₇X₁₈X₁₉HX₂₀X₂₁X₂₂X₂₃X₂₄X₂₅X₂₆X₂₇X₂₈X₂₉X₃₀ | |
| | | | FSRTDTLRDHQRTHTGEKPTGKK | | | |
| | | | TS | | | |
| gtgggggca | GTG GGG | 1193 | LEPGEKPYKCPECGKSFSRSDKL | 1742 | X₁X₂X₃X₄X₅X₆X₇X₈X₉CX₁₀X₁₁CX₁₂X₁₃X₁₄X₁₅X₁₆RSDKLVRHX₁₇X₁₈X₁₉ | 2291 |
| ggtgtgcct | GCA GGT | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TKNSLTEHX₁₇X₁₈ | |
| ggg | gtg CCT | | TKNSLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDELVRHX₁₇ | |
| | GGG | | GKSFSRSDELVRHQRTHTGEKPY | | X₁₈X₁₉HX₂₀X₂₁X₂₂X₂₃X₂₄X₈X₉CX₁₀X₁₁CX₁₂X₁₃X₁₄X15KX₁₆TSGHLVRH | |
| | | | KCPECGKSFSTSGHLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QSGDLR | |
| | | | GEKPYKCPECGKSFSQSGDLRRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDK | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | KLVRHQRTHTGEKPYKCPECGKS | | DELVRHX₁₇X₁₈X₁₉HX₂₀X₂₁X₂₂X₂₃X₂₄X₂₅X₂₆X₂₇X₂₈X₂₉X₃₀ | |
| | | | FSRSDELVRHQRTHTGEKPTGKK | | | |
| | | | TS | | | |
| ccagtgggg | CCA GTG | 1194 | LEPGEKPYKCPECGKSFSTKNSL | 1743 | X₁X₂X₃X₄X₅X₆X₇X₈X₉CX₁₀X₁₁CX₁₂X₁₃X₁₄X₁₅X₁₆TKNSLTEHX₁₇X₁₈X₁₉ | 2292 |
| gcaggtgtg | GGG GCA | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDELVRHX₁₇X₁₈ | |
| cct | GGT gtg | | RSDELVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSGHLVRHX₁₇ | |
| | CCT | | GKSFSTSGHLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QSGDLRRH | |
| | | | KCPECGKSFSQSGDLRRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDKLV | |
| | | | GEKPYKCPECGKSFSRSDKLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDE | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | ELVRHQRTHTGEKPYKCPECGKS | | HSLTEHX₁₇X₁₈X₁₉HX₂₀X₂₁X₂₂X₂₃X₂₄X₂₅X₂₆X₂₇X₂₈X₂₉X₃₀ | |
| | | | FSTSHSLTEHQRTHTGEKPTGKK | | | |
| | | | TS | | | |
| gtgccagtg | gtg CCA | 1195 | LEPGEKPYKCPECGKSFSRSDEL | 1744 | X₁X₂X₃X₄X₅X₆X₇X₈X₉CX₁₀X₁₁CX₁₂X₁₃X₁₄X₁₅X₁₆RSDELVRHX₁₇X₁₈X₁₉ | 2293 |
| ggggcaggt | GTG GGG | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSGHLVRHX₁₇X₁₈ | |
| gtg | GCA GGT | | TSGHLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QSGDLRRHX₁₇ | |
| | GTG | | GKSFSQSGDLRRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDKLVRH | |
| | | | KCPECGKSFSRSDKLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDELV | |
| | | | GEKPYKCPECGKSFSRSDELVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆TSHS | |
| | | | QRTHTGEKPYKCPECGKSFSTSH | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | SLTEHQRTHTGEKPYKCPECGKS | | DELVRHX₁₇X₁₈X₁₉HX₂₀X₂₁X₂₂X₂₃X₂₄X₂₅X₂₆X₂₇X₂₈X₂₉X₃₀ | |
| | | | FSRSDELVRHQRTHTGEKPTGKK | | | |
| | | | TS | | | |
| ccaggtgtg | CCA GGT | 1196 | LEPGEKPYKCPECGKSFSQSGDL | 1745 | X₁X₂X₃X₄X₅X₆X₇X₈X₉CX₁₀X₁₁CX₁₂X₁₃X₁₄X₁₅X₁₆QSGDLRRHX₁₇X₁₈X₁₉ | 2294 |
| ccagtgggg | gtg CCA | | RRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDKLVRHX₁₇X₁₈ | |
| gca | GTG GGG | | RSDKLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDELVRHX₁₇ | |
| | GCA | | GKSFSRSDELVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSHSLTEH | |
| | | | KCPECGKSFSTSHSLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDELV | |
| | | | GEKPYKCPECGKSFSRSDELVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆TSGH | |
| | | | QRTHTGEKPYKCPECGKSFSTSG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | HLVRHQRTHTGEKPYKCPECGKS | | HSLTEHX₁₇X₁₈X₁₉HX₂₀X₂₁X₂₂X₂₃X₂₄X₂₅X₂₆X₂₇X₂₈X₂₉X₃₀ | |
| | | | FSTSHSLTEHQRTHTGEKPTGKK | | | |
| | | | TS | | | |
| ggtgtgcca | GGT gtg | 1197 | LEPGEKPYKCPECGKSFSTSGHL | 1746 | X₁X₂X₃X₄X₅X₆X₇X₈X₉CX₁₀X₁₁CX₁₂X₁₃X₁₄X₁₅X₁₆TSGHLVRHX₁₇X₁₈X₁₉ | 2295 |
| gtgggggca | CCA GTG | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QSGDLRRHX₁₇X₁₈ | |
| ggt | GGG GCA | | QSGDLRRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDKLVRHX₁₇ | |
| | GGT | | GKSFSRSDKLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDELVRH | |
| | | | KCPECGKSFSRSDELVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSHSLT | |
| | | | GEKPYKCPECGKSFSTSHSLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆RSDE | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | ELVRHQRTHTGEKPYKCPECGKS | | GHLVRHX₁₇X₁₈X₁₉HX₂₀X₂₁X₂₂X₂₃X₂₄X₂₅X₂₆X₂₇X₂₈X₂₉X₃₀ | |
| | | | FSTSGHLVRHQRTHTGEKPTGKK | | | |
| | | | TS | | | |
| ccaggagca | CCA GGA | 1198 | LEPGEKPYKCPECGKSFSSKKAL | 1747 | X₁X₂X₃X₄X₅X₆X₇X₈X₉CX₁₀X₁₁CX₁₂X₁₃X₁₄X₁₅X₁₆SKKALTEHX₁₇X₁₈X₁₉ | 2296 |
| gatctttgg | GCA GAT | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDHLTTHX₁₇X₁₈ | |
| cac | CTT TGG | | RSDHLTTHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TTGALTEHX₁₇ | |
| | CAC | | GKSFSTTGALTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSGNLVRH | |
| | | | KCPECGKSFSTSGNLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QSGDLR | |
| | | | GEKPYKCPECGKSFSQSGDLRRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆QRAH | |
| | | | QRTHTGEKPYKCPECGKSFSQRA | | LERHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | HLERHQRTHTGEKPYKCPECGKS | | HSLTEHX₁₇X₁₈X₁₉HX₂₀X₂₁X₂₂X₂₃X₂₄X₂₅X₂₆X₂₇X₂₈X₂₉X₃₀ | |
| | | | FSTSHSLTEHQRTHTGEKPTGKK | | | |
| | | | TS | | | |
| ctgggtcca | CTG GGT | 1199 | LEPGEKPYKCPECGKSFSTTGAL | 1748 | X₁X₂X₃X₄X₅X₆X₇X₈X₉CX₁₀X₁₁CX₁₂X₁₃X₁₄X₁₅X₁₆TTGALTEHX₁₇X₁₈X₁₉ | 2297 |
| ggagcagat | CCA GGA | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSGNLVRHX₁₇X₁₈ | |
| ctt | GCA GAT | | TSGNLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QSGDLRRHX₁₇ | |
| | CTT | | GKSFSQSGDLRRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QRAHLERH | |
| | | | KCPECGKSFSQRAHLERHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSHSLT | |
| | | | GEKPYKCPECGKSFSTSHSLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆TSGH | |
| | | | QRTHTGEKPYKCPECGKSFSTSG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RN | |
| | | | HLVRHQRTHTGEKPYKCPECGKS | | DALTEHX₁₇X₁₈X₁₉HX₂₀X₂₁X₂₂X₂₃X₂₄X₂₅X₂₆X₂₇X₂₈X₂₉X₃₀ | |
| | | | FSRNDALTEHQRTHTGEKPTGKK | | | |
| | | | TS | | | |
| ggtccagga | GGT CCA | 1200 | LEPGEKPYKCPECGKSFSRSDHL | 1749 | X₁X₂X₃X₄X₅X₆X₇X₈X₉CX₁₀X₁₁CX₁₂X₁₃X₁₄X₁₅X₁₆RSDHLTTHX₁₇X₁₈X₁₉ | 2298 |
| gcagatctt | GGA GCA | | TTHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TTGALTEHX₁₇X₁₈ | |
| tgg | GAT CTT | | TTGALTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSGNLVRHX₁₇ | |
| | TGG | | GKSFSTSGNLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QSGDLRRH | |
| | | | KCPECGKSFSQSGDLRRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QRAHLE | |
| | | | GEKPYKCPECGKSFSQRAHLERH | | RHX₁₇X₁₈X₁₉HX₂₀X₂₁X₂₂X₂₃X₂₄X₈X₉CX₁₀X₁₁CX₁₂X₁₃X₁₄X₁₅X₁₆TSHS | |
| | | | QRTHTGEKPYKCPECGKSFSTSH | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | SLTEHQRTHTGEKPYKCPECGKS | | GHLVRHX₁₇X₁₈X₁₉HX₂₀X₂₁X₂₂X₂₃X₂₄X₂₅X₂₆X₂₇X₂₈X₂₉X₃₀ | |
| | | | FSTSGHLVRHQRTHTGEKPTGKK | | | |
| | | | TS | | | |
| gggaggaga | ggg AGG | 1201 | LEPGEKPYKCPECGKSFSTTGNL | 1750 | X₁X₂X₃X₄X₅X₆X₇X₈X₉CX₁₀X₁₁CX₁₂X₁₃X₁₄X₁₅X₁₆TTGNLTVHX₁₇X₁₈X₁₉ | 2299 |
| atgatacaa | AGA ATG | | TVHQRTHTGEKPYKCPECGKSFS | | 9HX₂₀X₂₁X₂₂X₂₃X₂₄X₈X₉CX₁₀X₁₁CX₁₂X₁₃X₁₄X₁₅X₁₆QSGNLTEHX₁₇X₁₈ | |
| aat | ATA CAA | | QSGNLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QKSSLIAHX₁₇ | |
| | AAT | | GKSFSQKSSLIAHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RRDELNVH | |
| | | | KCPECGKSFSRRDELNVHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QLAHLR | |
| | | | GEKPYKCPECGKSFSQLAHLRAH | | AHX₁₇X₁₈X₁₉HX₂₀X₂₁X₂₂X₂₃X₂₄X₈X₉CX₁₀X₁₁CX₁₂X₁₃X₁₄X₁₅X₁₆RSDH | |
| | | | QRTHTGEKPYKCPECGKSFSRSD | | LTNHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | HLTNHQRTHTGEKPYKCPECGKS | | DKLVRHX₁₇X₁₈X₁₉HX₂₀X₂₁X₂₂X₂₃X₂₄X₂₅X₂₆X₂₇X₂₈X₂₉X₃₀ | |
| | | | FSRSDKLVRHQRTHTGEKPTGKK | | | |
| | | | TS | | | |
| tggggtggg | TGG GGT | 1202 | LEPGEKPYKCPECGKSFSQKSSL | 1751 | X₁X₂X₃X₄X₅X₆X₇X₈X₉CX₁₀X₁₁CX₁₂X₁₃X₁₄X₁₅X₁₆QKSSLIAHX₁₇X₁₈X₁₉ | 2300 |
| aggagaatg | ggg AGG | | IAHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RRDELNVHX₁₇X₁₈ | |
| ata | AGA ATG | | RRDELNVHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QLAHLRAHX₁₇ | |
| | ATA | | GKSFSQLAHLRAHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDHLTNH | |
| | | | KCPECGKSFSRSDHLTNHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDKLV | |
| | | | GEKPYKCPECGKSFSRSDKLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆TSGH | |
| | | | QRTHTGEKPYKCPECGKSFSTSG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | HLVRHQRTHTGEKPYKCPECGKS | | DHLTTHX₁₇X₁₈X₁₉HX₂₀X₂₁X₂₂X₂₃X₂₄X₂₅X₂₆X₂₇X₂₈X₂₉X₃₀ | |
| | | | FSRSDHLTTHQRTHTGEKPTGKK | | | |
| | | | TS | | | |
| ctttggggt | CTT TGG | 1203 | LEPGEKPYKCPECGKSFSRRDEL | 1752 | X₁X₂X₃X₄X₅X₆X₇X₈X₉CX₁₀X₁₁CX₁₂X₁₃X₁₄X₁₅X₁₆RRDELNVHX₁₇X₁₈X₁₉ | 2301 |
| gggaggaga | GGT ggg | | NVHQRTHTGEKPYKCPECGKSFS | | HX₂₀X₂₁X₂₂X₂₃X₂₄X₈X₉CX₁₀X₁₁CX₁₂X₁₁X₁₃X₁₄X₁₅X₁₆QLAHLRAHX₁₇X₁₈ | |
| atg | AGG AGA | | QLAHLRAHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDHLTNHX₁₇ | |
| | ATG | | GKSFSRSDHLTNHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDKLVRH | |
| | | | KCPECGKSFSRSDKLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSGHLV | |
| | | | GEKPYKCPECGKSFSTSGHLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDH | |
| | | | QRTHTGEKPYKCPECGKSFSRSD | | LTTHX₁₇X₁₈X₁₉HX₂₀X₂₁X₂₂X₂₃X₂₄X₈X₉CX₁₀X₁₁CX₁₂X₁₃X₁₄X₁₅X₁₆TT | |
| | | | HLTTHQRTHTGEKPYKCPECGKS | | GALTEHX₁₇X₁₈X₁₉HX₂₀X₂₁X₂₂X₂₃X₂₄X₂₅X₂₆X₂₇X₂₈X₂₉X₃₀ | |
| | | | FSTTGALTEHQRTHTGEKPTGKK | | | |
| | | | TS | | | |
| ggcactcaa | GGC ACT | 1204 | LEPGEKPYKCPECGKSFSRSDKL | 1753 | X₂X₃X₄X₅X₆X₇X₈X₉CX₁₀X₁₁CX₁₂X₁₃X₁₄X₁₅X₁₆RSDKLVRHX₁₇X₁₈X₁₉ | 2302 |
| ctttggggt | CAA CTT | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSGHLVRHX₁₇X₁₈ | |
| ggg | TGG GGT | | TSGHLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDHLTTHX₁₇ | |
| | GGG | | GKSFSRSDHLTTHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TTGALTEH | |
| | | | KCPECGKSFSTTGALTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QSGNLT | |
| | | | GEKPYKCPECGKSFSQSGNLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆THLD | |
| | | | QRTHTGEKPYKCPECGKSFSTHL | | LIRHX₁₇X₁₈X₁₉HX₂₀X₂₁X₂₂X₂₃X₂₄X₈X₉CX₁₀X₁₁CX₁₂X₁₃X₁₄X₁₅X₁₆DP | |
| | | | DLIRHQRTHTGEKPYKCPECGKS | | GHLVRHX₁₇X₁₈X₁₉HX₂₀X₂₁X₂₂X₂₃X₂₄X₂₅X₂₆X₂₇X₂₈X₂₉X₃₀ | |
| | | | FSDPGHLVRHQRTHTGEKPTGKK | | | |
| | | | TS | | | |
| aggagaatg | AGG AGA | 1205 | LEPGEKPYKCPECGKSFSTSGHL | 1754 | X₁X₂X₃X₄X₅X₆X₇X₈X₉CX₁₀X₁₁CX₁₂X₁₃X₁₄X₁₅X₁₆TSGHLVRHX₁₇X₁₈X₁₉ | 2303 |
| atacaaaat | ATG ATA | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TTGNLTVHX₁₇X₁₈ | |
| ggt | CAA AAT | | TTGNLTVHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QSGNLTEHX₁₇ | |
| | GGT | | GKSFSQSGNLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QKSSLIAH | |
| | | | KCPECGKSFSQKSSLIAHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RRDELN | |
| | | | GEKPYKCPECGKSFSRRDELNVH | | VHX₁₇X₁₈X₁₉HX₂₀X₂₁X₂₂X₂₃X₂₄X₈X₉CX₁₀X1CX₁₂X₁₃X₁₄X₁₅X₁₆QLAH | |
| | | | QRTHTGEKPYKCPECGKSFSQLA | | LRAHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | HLRAHQRTHTGEKPYKCPECGKS | | DHLTNHX₁₇X₁₈X₁₉HX₂₀X₂₁X₂₂X₂₃X₂₄X₂₅X₂₆X₂₇X₂₈X₂₉X₃₀ | |
| | | | FSRSDHLTNHQRTHTGEKPTGKK | | | |
| | | | TS | | | |
| agaatgata | AGA ATG | 1206 | LEPGEKPYKCPECGKSFSRSDHL | 1755 | X₁X₂X₃X₄X₅X₆X₇X₈X₉CX₁₀X₁₁CX₁₂X₁₃X₁₄X₁₅X₁₆RSDHLTNHX₁₇X₁₈X₁₉ | 2304 |
| caaaatggt | ATA CAA | | TNHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSGHLVRHX₁₇X₁₈ | |
| agg | AAT GGT | | TSGHLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TTGNLTVHX₁₇ | |
| | AGG | | GKSFSTTGNLTVHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QSGNLTEH | |
| | | | KCPECGKSFSQSGNLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QKSSLI | |
| | | | GEKPYKCPECGKSFSQKSSLIAH | | AHX₁₇X₁₈X₁₉HX₂₀X₂₁X₂₂X₂₃X₂₄X₈X₉CX₁₀X₁₁CX₁₂X₁₃X₁₄X₁₅X₁₆RRDE | |
| | | | QRTHTGEKPYKCPECGKSFSRRD | | LNVHX₁₇X₁₈X₁₉HX₂₀X₂₁X₂₂X₂₃X₂₄X₈X₉CX₁₀X₁₁CX₁₂X₁₃X₁₄X₁₅X₁₆QL | |
| | | | ELNVHQRTHTGEKPYKCPECGKS | | AHLRAHX₁₇X₁₈X₁₉HX₂₀X₂₁X₂₂X₂₃X₂₄X₂₅X₂₆X₂₇X₂₈X₂₉X₃₀ | |
| | | | FSQLAHLRAHQRTHTGEKPTGKK | | | |
| | | | TS | | | |
| ggtgggagg | GGT ggg | 1207 | LEPGEKPYKCPECGKSFSQSGNL | 1756 | X₁X₂X₃X₄X₅X₆X₇X₈X₉CX₁₀X₁₁CX₁₂X₁₃X₁₄X₁₅X₁₆QSGNLTEHX₁₇X₁₈X₁₉ | 2305 |
| agaatgata | AGG AGA | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QKSSLIAHX₁₇X₁₈ | |
| caa | ATG ATA | | QKSSLIAHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RRDELNVHX₁₇ | |
| | CAA | | GKSFSRRDELNVHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QLAHLRAH | |
| | | | KCPECGKSFSQLAHLRAHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDHLT | |
| | | | GEKPYKCPECGKSFSRSDHLTNH | | NHX₁₇X₁₈X₁₉HX₂₀X₂₁X₂₂X₂₃X₂₄X₈X₉CX₁₀X₁₁CX₁₂X₁₃X₁₄X₁₅X₁₆RSDK | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | KLVRHQRTHTGEKPYKCPECGKS | | GHLVRHX₁₇X₁₈X₁₉HX₂₀X₂₁X₂₂X₂₃X₂₄X₂₅X₂₆X₂₇X₂₈X₂₉X₃₀ | |
| | | | FSTSGHLVRHQRTHTGEKPTGKK | | | |
| | | | TS | | | |
| caactttgg | CAA CTT | 1208 | LEPGEKPYKCPECGKSFSQLAHL | 1757 | X₁X₂X₃X₄X₅X₆X₇X₈X₉CX₁₀X₁₁CX₁₂X₁₃X₁₄X₁₅X₁₆QLAHLRAHX₁₇X₁₈X₁₉ | 2306 |
| ggtgggagg | TGG GGT | | RAHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDHLTNHX₁₇X₁₈ | |
| aga | ggg AGG | | RSDHLTNHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDKLVRHX₁₇ | |
| | AGA | | GKSFSRSDKLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSGHLVRH | |
| | | | KCPECGKSFSTSGHLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDHLT | |
| | | | GEKPYKCPECGKSFSRSDHLTTH | | THX₁₇X₁₈X₁₉HX₂₀X₂₁X₂₂X₂₃X₂₄X₈X₉CX₁₀X₁₁CX₁₂X₁₃X₁₄X₁₅X₁₆TTGA | |
| | | | QRTHTGEKPYKCPECGKSFSTTG | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆QS | |
| | | | ALTEHQRTHTGEKPYKCPECGKS | | GNLTEHX₁₇X₁₈X₁₉HX₂₀X₂₁X₂₂X₂₃X₂₄X₂₅X₂₆X₂₇X₂₈X₂₉X₃₀ | |
| | | | FSQSGNLTEHQRTHTGEKPTGKK | | | |
| | | | TS | | | |
| actcaactt | ACT CAA | 1209 | LEPGEKPYKCPECGKSFSRSDHL | 1758 | X₁X₂X₃X₄X₅X₆X₇X₈X₉CX₁₀X₁₁CX₁₂X₁₃X₁₄X₁₅X₁₆RSDHLTNHX₁₇X₁₈X₁₉ | 2307 |
| tggggtggg | CTT TGG | | TNHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDKLVRHX₁₇X₁₈ | |
| agg | GGT ggg | | RSDKLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSGHLVRHX₁₇ | |
| | AGG | | GKSFSTSGHLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDHLTTH | |
| | | | KCPECGKSFSRSDHLTTHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TTGALT | |
| | | | GEKPYKCPECGKSFSTTGALTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆QSGN | |
| | | | QRTHTGEKPYKCPECGKSFSQSG | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆TH | |
| | | | NLTEHQRTHTGEKPYKCPECGKS | | LDLIRHX₁₇X₁₈X₁₉HX₂₀X₂₁X₂₂X₂₃X₂₄X₂₅X₂₆X₂₇X₂₈X₂₉X₃₀ | |
| | | | FSTHLDLIRHQRTHTGEKPTGKK | | | |
| | | | TS | | | |
| gggtgggag | GGG TGG | 1210 | LEPGEKPYKCPECGKSFSSPADL | 1759 | X₁X₂X₃X₄X₅X₆X₇X₈X₉CX₁₀X₁₁CX₁₂X₁₃X₁₄X₁₅X₁₆SPADLTRHX₁₇X₁₈X₁₉ | 2308 |
| gagaatgat | GAG gag | | TRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSGNLVRHX₁₇X₁₈ | |
| aca | AAT GAT | | TSGNLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TTGNLTVHX₁₇ | |
| | ACA | | GKSFSTTGNLTVHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDNLVRH | |
| | | | KCPECGKSFSRSDNLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDNLV | |
| | | | GEKPYKCPECGKSFSRSDNLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDH | |
| | | | QRTHTGEKPYKCPECGKSFSRSD | | LTTHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | HLTTHQRTHTGEKPYKCPECGKS | | DKLVRHX₁₇X₁₈X₁₉HX₂₀X₂₁X₂₂X₂₃X₂₄X₂₅X₂₆X₂₇X₂₈X₂₉X₃₀ | |
| | | | FSRSDKLVRHQRTHTGEKPTGKK | | | |
| | | | TS | | | |
| gagaatgat | gag AAT | 1211 | LEPGEKPYKCPECGKSFSREDNL | 1760 | X₁X₂X₃X₄X₅X₆X₇X₈X₉CX₁₀X₁₁CX₁₂X₁₃X₁₄X₁₅X₁₆REDNLHTHX₁₇X₁₈X₁₉ | 2309 |
| acaaaatgg | GAT ACA | | HTHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDHLTTHX₁₇X₁₈ | |
| tag | AAA TGG | | RSDHLTTHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QRANLRAHX₁₇ | |
| | TAG | | GKSFSQRANLRAHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆SPADLTRH | |
| | | | KCPECGKSFSSPADLTRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSGNLV | |
| | | | GEKPYKCPECGKSFSTSGNLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆TTGN | |
| | | | QRTHTGEKPYKCPECGKSFSTTG | | LTVHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | NLTVHQRTHTGEKPYKCPECGKS | | DNLVRHX₁₇X₁₈X₁₉HX₂₀X₂₁X₂₂X₂₃X₂₄X₂₅X₂₆X₂₇X₂₈X₂₉X₃₀ | |
| | | | FSRSDNLVRHQRTHTGEKPTGKK | | | |
| | | | TS | | | |
| tgggaggag | TGG GAG | 1212 | LEPGEKPYKCPECGKSFSQRANL | 1761 | X₁X₂X₃X₄X₅X₆X₇X₈X₉CX₁₀X₁₁CX₁₂X₁₃X₁₄X₁₅X₁₆QRANLRAHX₁₇X₁₈X₁₉ | 2310 |
| aatgataca | gag AAT | | RAHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆SPADLTRHX₁₇X₁₈ | |
| aaa | GAT ACA | | SPADLTRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSGNLVRHX₁₇ | |
| | AAA | | GKSFSTSGNLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TTGNLTVH | |
| | | | KCPECGKSFSTTGNLTVHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDNLV | |
| | | | GEKPYKCPECGKSFSRSDNLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDN | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | NLVRHQRTHTGEKPYKCPECGKS | | DHLTTHX₁₇X₁₈X₁₉HX₂₀X₂₁X₂₂X₂₃X₂₄X₂₅X₂₆X₂₇X₂₈X₂₉X₃₀ | |
| | | | FSRSDHLTTHQRTHTGEKPTGKK | | | |
| | | | TS | | | |
| gaggagaat | GAG gag | 1213 | LEPGEKPYKCPECGKSFSRSDHL | 1762 | X₁X₂X₃X₄X₅X₆X₇X₈X₉CX₁₀X₁₁CX₁₂X₁₃X₁₄X₁₅X₁₆RSDHLTTHX₁₇X₁₈X₁₉ | 2311 |
| gatacaaaa | AAT GAT | | TTHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QRANLRAHX₁₇X₁₈ | |
| tgg | ACA AAA | | QRANLRAHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆SPADLTRHX₁₇ | |
| | TGG | | GKSFSSPADLTRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSGNLVRH | |
| | | | KCPECGKSFSTSGNLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TTGNLT | |
| | | | GEKPYKCPECGKSFSTTGNLTVH | | VHX₁₇X₁₈X₁₉HX₂₀X₂₁X₂₂X₂₃X₂₄X₈X₉CX₁₀X₁₁CX₁₂X₁₃X₁₄X₁₅X₁₆RSDN | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | NLVRHQRTHTGEKPYKCPECGKS | | DNLVRHX₁₇X₁₈X₁₉HX₂₀X₂₁X₂₂X₂₃X₂₄X₂₅X₂₆X₂₇X₂₈X₂₉X₃₀ | |
| | | | FSRSDNLVRHQRTHTGEKPTGKK | | | |
| | | | TS | | | |
| aatgataca | AAT GAT | 1214 | LEPGEKPYKCPECGKSFSTSGSL | 1763 | X₁X₂X₃X₄X₅X₆X₇X₈X₉CX₁₀X₁₁CX₁₂X₁₃X₁₄X₁₅X₁₆TSGSLVRHX₁₇X₁₈X₁₉ | 2312 |
| aaatggtag | ACA AAA | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆REDNLHTHX₁₇X₁₈ | |
| gtt | TGG TAG | | REDNLHTHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDHLTTHX₁₇ | |
| | GTT | | GKSFSRSDHLTTHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QRANLRAH | |
| | | | KCPECGKSFSQRANLRAHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆SPADLT | |
| | | | GEKPYKCPECGKSFSSPADLTRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆TSGN | |
| | | | QRTHTGEKPYKCPECGKSFSTSG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆TT | |
| | | | NLVRHQRTHTGEKPYKCPECGKS | | GNLTVHX₁₇X₁₈X₁₉HX₂₀X₂₁X₂₂X₂₃X₂₄X₂₅X₂₆X₂₇X₂₈X₂₉X₃₀ | |
| | | | FSTTGNLTVHQRTHTGEKPTGKK | | | |
| | | | TS | | | |
| ggtcctaca | GGT CCT | 1215 | LEPGEKPYKCPECGKSFSRSDHL | 1764 | X₁X₂X₃X₄X₅X₆X₇X₈X₉CX₁₀X₁₁CX₁₂X₁₃X₁₄X₁₅X₁₆RSDHLTNHX₁₇X₁₈X₁₉ | 2313 |
| ggccagcac | ACA GGC | | TNHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆SKKALTEHX₁₇X₁₈ | |
| agg | CAG CAC | | SKKALTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RADNLTEHX₁₇ | |
| | AGG | | GKSFSRADNLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DPGHLVRH | |
| | | | KCPECGKSFSDPGHLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆SPADLT | |
| | | | GEKPYKCPECGKSFSSPADLTRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆TKNS | |
| | | | QRTHTGEKPYKCPECGKSFSTKN | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | SLTEHQRTHTGEKPYKCPECGKS | | GHLVRHX₁₇X₁₈X₁₉HX₂₀X₂₁X₂₂X₂₃X₂₄X₂₅X₂₆X₂₇X₂₈X₂₉X₃₀ | |
| | | | FSTSGHLVRHQRTHTGEKPTGKK | | | |
| | | | TS | | | |
| taggttggt | TAG GTT | 1216 | LEPGEKPYKCPECGKSFSRADNL | 1765 | X₁X₂X₃X₄X₅X₆X₇X₈X₉CX₁₀X₁₁CX₁₂X₁₃X₁₄X₁₅X₁₆RADNLTEHX₁₇X₁₈X₁₉ | 2314 |
| cctacaggc | GGT CCT | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆DPGHLVRHX₁₇X₁₈ | |
| cag | ACA GGC | | DPGHLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆SPADLTRHX₁₇ | |
| | CAG | | GKSFSSPADLTRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TKNSLTEH | |
| | | | KCPECGKSFSTKNSLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSGHLV | |
| | | | GEKPYKCPECGKSFSTSGHLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆TSGS | |
| | | | QRTHTGEKPYKCPECGKSFSTSG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RE | |
| | | | SLVRHQRTHTGEKPYKCPECGKS | | DNLHTHX₁₇X₁₈X₁₉HX₂₀X₂₁X₂₂X₂₃X₂₄X₂₅X₂₆X₂₇X₂₈X₂₉X₃₀ | |
| | | | FSREDNLHTHQRTHTGEKPTGKK | | | |
| | | | TS | | | |
| tggtaggtt | TGG TAG | 1217 | LEPGEKPYKCPECGKSFSDPGHL | 1766 | X₁X₂X₃X₄X₅X₆X₇X₈X₉CX₁₀X₁₁CX₁₂X₁₃X₁₄X₁₅X₁₆DPGHLVRHX₁₇X₁₈X₁₉ | 2315 |
| ggtcctaca | GTT GGT | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆SPADLTRHX₁₇X₁₈ | |
| ggc | CCT ACA | | SPADLTRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TKNSLTEHX₁₇ | |
| | GGC | | GKSFSTKNSLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSGHLVRH | |
| | | | KCPECGKSFSTSGHLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSGSLV | |
| | | | GEKPYKCPECGKSFSTSGSLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆REDN | |
| | | | QRTHTGEKPYKCPECGKSFSRED | | LHTHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | NLHTHQRTHTGEKPYKCPECGKS | | DHLTTHX₁₇X₁₈X₁₉HX₂₀X₂₁X₂₂X₂₃X₂₄X₂₅X₂₆X₂₇X₂₈X₂₉X₃₀ | |
| | | | FSRSDHLTTHQRTHTGEKPTGKK | | | |
| | | | TS | | | |
| aaatggtag | AAA TGG | 1218 | LEPGEKPYKCPECGKSFSSPADL | 1767 | X₁X₂X₃X₄X₅X₆X₇X₈X₉CX₁₀X₁₁CX₁₂X₁₃X₁₄X₁₅X₁₆SPADLTRHX₁₇X₁₈X₁₉ | 2316 |
| gttggtcct | TAG GTT | | TRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TKNSLTEHX₁₇X₁₈ | |
| aca | GGT CCT | | TKNSLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSGHLVRHX₁₇ | |
| | GAC | | GKSFSTSGHLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSGSLVRH | |
| | | | KCPECGKSFSTSGSLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆REDNLH | |
| | | | GEKPYKCPECGKSFSREDNLHTH | | THX₁₇X₁₈X₁₉HX₂₀X₂₁X₂₂X₂₃X₂₄X₈X₉CX₁₀X₁₁CX₁₂X₁₃X₁₄X₁₅X₁₆RSDH | |
| | | | ARTHTGEKPYKCPECGKSFSRSD | | LTTHX₁₇X₁₈X₁₉HX₂₀X₂₁X₂₂X₂₃X₂₄X₈X₉CX₁₀X₁₁CX₁₂X₁₃X₁₄X₁₅X₁₆QR | |
| | | | HLTTHQRTHTGEKPYKCPECGKS | | ANLRAHX₁₇X₁₈X₁₉HX₂₀X₂₁X₂₂X₂₃X₂₄X₂₅X₂₆X₂₇X₂₈X₂₉X₃₀ | |
| | | | FSQRANLRAHQRTHTGEKPTGKK | | | |
| | | | TS | | | |
| acaaaatgg | ACA AAA | 1219 | LEPGEKPYKCPECGKSFSTKNSL | 1768 | X₁X₂X₃X₄X₅X₆X₇X₈X₉CX₁₀X₁₁CX₁₂X₁₃X₁₄X₁₅X₁₆TKNSLTEHX₁₇X₁₈X₁₉ | 2317 |
| taggttggt | TGG TAG | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSGHLVRHX₁₇X₁₈ | |
| cct | GTT GGT | | TSGHLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSGSLVRHX₁₇ | |
| | CCT | | GKSFSTSGSLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆REDNLHTH | |
| | | | KCPECGKSFSREDNLHTHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDHLT | |
| | | | GEKPYKCPECGKSFSRSDHLTTH | | THX₁₇X₁₈X₁₉HX₂₀X₂₁X₂₂X₂₃X₂₄X₈X₉CX₁₀X₁₁CX₁₂X₁₃X₁₄X₁₅X₁₆QRAN | |
| | | | QRTHTGEKPYKCPECGKSFSQRA | | LRAHX₁₇X₁₈X₁₉HX₂₀X₂₁X₂₂X₂₃X₂₄X₈X₉CX₁₀X₁₁CX₁₂X₁₃X₁₄X₁₅X₁₆SP | |
| | | | NLRAHQRTHTGEKPYKCPECGKS | | ADLTRHX₁₇X₁₈X₁₉HX₂₀X₂₁X₂₂X₂₃X₂₄X₂₅X₂₆X₂₇X₂₈X₂₉X₃₀ | |
| | | | FSSPADLTRHQRTHTGEKPTGKK | | | |
| | | | TS | | | |
| gatacaaaa | GAT ACA | 1220 | LEPGEKPYKCPECGKSFSTSGHL | 1769 | X₁X₂X₃X₄X₅X₆X₇X₈X₉CX₁₀X₁₁CX₁₂X₁₃X₁₄X₁₅X₁₆TSGHLVRHX₁₇X₁₈X₁₉ | 2318 |
| tggtaggtt | AAA TGG | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSGSLVRHX₁₇X₁₈ | |
| ggt | TAG GTT | | TSGSLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆REDNLHTHX₁₇ | |
| | GGT | | GKSFSREDNLHTHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDHLTTH | |
| | | | KCPECGKSFSRSDHLTTHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QRANLR | |
| | | | GEKPYKCPECGKSFSQRANLRAH | | AHX₁₇X₁₈X₁₉HX₂₀X₂₁X₂₂X₂₃X₂₄X₈X₉CX₁₀X₁₁CX₁₂X₁₃X₁₄X₁₅X₁₆SPAD | |
| | | | QRTHTGEKPYKCPECGKSFSSPA | | LTRHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | DLTRHQRTHTGEKPYKCPECGKS | | GNLVRHX₁₇X₁₈X₁₉HX₂₀X₂₁X₂₂X₂₃X₂₄X₂₅X₂₆X₂₇X₂₈X₂₉X₃₀ | |
| | | | FSTSGNLVRHQRTHTGEKPTGKK | | | |
| | | | TS | | | |
| gttggtcct | GTT GGT | 1221 | LEPGEKPYKCPECGKSFSSKKAL | 1770 | X₁X₂X₃X₄X₅X₆X₇X₈X₉CX₁₀X₁₁CX₁₂X₁₃X₁₄X₁₅X₁₆SKKALTEHX₁₇X₁₈X₁₉ | 2319 |
| acaggccag | CCT ACA | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RADNLTEHX₁₇X₁₈ | |
| cac | GGC CAG | | RADNLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DPGHLVRHX₁₇ | |
| | CAC | | GKSFSDPGHLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆SPADLTRH | |
| | | | KCPECGKSFSSPADLTRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TKNSLT | |
| | | | GEKPYKCPECGKSFSTKNSLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆TSGH | |
| | | | QRTHTGEKPYKCPECGKSFSTSG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | HLVRHQRTHTGEKPYKCPECGKS | | GSLVRHX₁₇X₁₈X₁₉HX₂₀X₂₁X₂₂X₂₃X₂₄X₂₅X₂₆X₂₇X₂₈X₂₉X₃₀ | |
| | | | FSTSGSLVRHQRTHTGEKPTGKK | | | |
| | | | TS | | | |
| gtcctacag | GTC CTA | 1222 | LEPGEKPYKCPECGKSFSTSGHL | 1771 | X₁X₂X₃X₄X₅X₆X₇X₈X₉CX₁₀X₁₁CX₁₂X₁₃X₁₄X₁₅X₁₆TSGHLVRHX₁₇X₁₈X₁₉ | 2320 |
| gccagcaca | CAG GCC | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆SPADLTRHX₁₇X₁₈ | |
| ggt | AGC ACA | | SPADLTRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆ERSHLREHX₁₇ | |
| | GGT | | GKSFSERSHLREHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DCRDLARH | |
| | | | KCPECGKSFSDCRDLARHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RADNLT | |
| | | | GEKPYKCPECGKSFSRADNLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆QNST | |
| | | | QRTHTGEKPYKCPECGKSFSQNS | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆DP | |
| | | | TLTEHQRTHTGEKPYKCPECGKS | | GALVRHX₁₇X₁₈X₁₉HX₂₀X₂₁X₂₂X₂₃X₂₄X₂₅X₂₆X₂₇X₂₈X₂₉X₃₀ | |
| | | | FSDPGALVRHQRTHTGEKPTGKK | | | |
| | | | TS | | | |
| caggccagc | CAG GCC | 1223 | LEPGEKPYKCPECGKSFSDCRDL | 1772 | X₁X₂X₃X₄X₅X₆X₇X₈X₉CX₁₀X₁₁CX₁₂X₁₃X₁₄X₁₅X₁₆DCRDLARHX₁₇X₁₈X₁₉ | 2321 |
| acaggtgtt | AGC ACA | | ARHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSGSLVRHX₁₇X₁₈ | |
| gcc | GGT GTT | | TSGSLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSGHLVRHX₁₇ | |
| | GCC | | GKSFSTSGHLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆SPADLTRH | |
| | | | KCPECGKSFSSPADLTRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆ERSHLR | |
| | | | GEKPYKCPECGKSFSERSHLREH | | EHX₁₇X₁₈X₁₉HX₂₀X₂₁X₂₂X₂₃X₂₄X₈X₉CX₁₀X₁₁CX₁₂X₁₃X₁₄X₁₅X₁₆DCRD | |
| | | | QRTHTGEKPYKCPECGKSFSDCR | | LARHX₁₇X₁₈X₁₉HX₂₀X₂₁X₂₂X₂₃X₂₄X₈X₉CX₁₀X₁₁CX₁₂X₁₃X₁₄X₁₅X₁₆RA | |
| | | | DLARHQRTHTGEKPYKCPECGKS | | DNLTEHX₁₇X₁₈X₁₉HX₂₀X₂₁X₂₂X₂₃X₂₄X₂₅X₂₆X₂₇X₂₈X₂₉X₃₀ | |
| | | | FSRADNLTEHQRTHTGEKPTGKK | | | |
| | | | TS | | | |
| ctacaggcc | CTA CAG | 1224 | LEPGEKPYKCPECGKSFSTSGSL | 1773 | X₁X₂X₃X₄X₅X₆X₇X₈X₉CX₁₀X₁₁CX₁₂X₁₃X₁₄X₁₅X₁₆TSGSLVRHX₁₇X₁₈X₁₉ | 2322 |
| agcacaggt | GCC AGC | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSGHLVRHX₁₇X₁₈ | |
| gtt | ACA GGT | | TSGHLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆SPADLTRHX₁₇ | |
| | GTT | | GKSFSSPADLTRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆ERSHLREH | |
| | | | KCPECGKSFSERSHLREHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DCRDLA | |
| | | | GEKPYKCPECGKSFSDCRDLARH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RADN | |
| | | | QRTHTGEKPYKCPECGKSFSRAD | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆QN | |
| | | | NLTEHQRTHTGEKPYKCPECGKS | | STLTEHX₁₇X₁₈X₁₉HX₂₀X₂₁X₂₂X₂₃X₂₄X₂₅X₂₆X₂₇X₂₈X₂₉X₃₀ | |
| | | | FSQNSTLTEHQRTHTGEKPTGKK | | | |
| | | | TS | | | |
| ggtgttgcc | GGT GTT | 1225 | LEPGEKPYKCPECGKSFSTSHSL | 1774 | X₁X₂X₃X₄X₅X₆X₇X₈X₉CX₁₀X₁₁CX₁₂X₁₃X₁₄X₁₅X₁₆TSHSLTEHX₁₇X₁₈X₁₉ | 2323 |
| aagtgaagc | GCC AAG | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆ERSHLREHX₁₇X₁₈ | |
| cca | TGA AGC | | ERSHLREHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QAGHLASHX₁₇ | |
| | CCA | | GKSFSQAGHLASHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RKDNLKNH | |
| | | | KCPECGKSFSRKDNLKNHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DCRDLA | |
| | | | GEKPYKCPECGKSFSDCRDLARH | | RHX₁₇X₁₈X₁₉HX₂₀X₂₁X₂₂X₂₃X₂₄X₈X₉CX₁₀X₁₁CX₁₂X₁₃X₁₄X₁₅X₁₆TSGS | |
| | | | QRTHTGEKPYKCPECGKSFSTSG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | SLVRHQRTHTGEKPYKCPECGKS | | GHLVRHX₁₇X₁₈X₁₉HX₂₀X₂₁X₂₂X₂₃X₂₄X₂₅X₂₆X₂₇X₂₈X₂₉X₃₀ | |
| | | | FSTSGHLVRHQRTHTGEKPTGKK | | | |
| | | | TS | | | |
| agcacaggt | AGC ACA | 1226 | LEPGEKPYKCPECGKSFSQAGHL | 1775 | X₁X₂X₃X₄X₅X₆X₇X₈X₉CX₁₀X₁₁CX₁₂X₁₃X₁₄X₁₅X₁₆QAGHLASHX₁₇X₁₈X₁₉ | 2324 |
| gttgccaag | GGT GTT | | ASHQRTHTGEKPYKCPECGKSFS | | HX₂₀X₂₁X₂₂X₂₃X₂₄X₈X₉CX₁₀XCX₁₂X₁₃X₁₄X₁₅X₁₆RKDNLKNHX₁₇X₁₈ | |
| tga | GCC AAG | | RKDNLKNHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DCRDLARHX₁₇ | |
| | TGA | | GKSFSDCRDLARHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSGSLVRH | |
| | | | KCPECGKSFSTSGSLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSGHLV | |
| | | | GEKPYKCPECGKSFSTSGHLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆SPAD | |
| | | | QRTHTGEKPYKCPECGKSFSSPA | | LTRHX₁₇X₁₈X₁₉HX₂₀X₂₁X₂₂X₂₃X₂₄X₈X₉CX₁₀X₁₁CX₁₂X₁₃X₁₄X₁₅X₁₆ER | |
| | | | DLTRHQRTHTGEKPYKCPECGKS | | SSHLREHX₁₇X₁₈X₁₉HX₂₀X₂₁X₂₂X₂₃X₂₄X₂₅X₂₆X₂₇X₂₈X₂₉X₃₀ | |
| | | | FSERSHLREHQRTHTGEKPTGKK | | | |
| | | | TS | | | |
| acaggtgtt | ACA GGT | 1227 | LEPGEKPYKCPECGKSFSERSHL | 1776 | X₁X₂X₃X₄X₅X₆X₇X₈X₉CX₁₀X₁₁CX₁₂X₁₃X₁₄X₁₅X₁₆ERSHLREHX₁₇X₁₈X₁₉ | 2325 |
| gccaagtga | GTT GCC | | REHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QAGHLASHX₁₇X₁₈ | |
| agc | AAG TGA | | QAGHLASHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RKDNLKNHX₁₇ | |
| | AGC | | GKSFSRKDNLKNHQRTHTGEKPY | | X₈X₁₉HX₂₀X₂₁X₂₂X₂₃X₂₄X₈X₉CX₁₀X₁₁CX₁₂X₁₃X₁₄X₁₅X₁₆DCRDLARH | |
| | | | KCPECGKSFSDCRDLARHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSGSLV | |
| | | | GEKPYKCPECGKSFSTSGSLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆TSGH | |
| | | | QRTHTGEKPYKCPECGKSFSTSG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆SP | |
| | | | HLVRHQRTHTGEKPYKCPECGKS | | ADLTRHX₁₇X₁₈X₁₉HX₂₀X₂₁X₂₂X₂₃X₂₄X₂₅X₂₆X₂₇X₂₈X₂₉X₃₀ | |
| | | | FSSPADLTRHQRTHTGEKPTGKK | | | |
| | | | TS | | | |
| gccagcaca | GCC AGC | 1228 | LEPGEKPYKCPECGKSFSRKDNL | 1777 | X₁X₂X₃X₄X₅X₆X₇X₈X₉CX₁₀X₁₁CX₁₂X₁₃X₁₄X₁₅X₁₆RKDNLKNHX₁₇X₁₈X₁₉ | 2326 |
| ggtgttgcc | ACA GGT | | KNHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆DCRDLARHX₁₇X₁₈ | |
| aag | GTT GCC | | DCRDLARHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSGSLVRHX₁₇ | |
| | APG | | GKSFSTSGSLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSGHLVRH | |
| | | | KCPECGKSFSTSGHLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆SPADLT | |
| | | | GEKPYKCPECGKSFSSPADLTRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆ERSH | |
| | | | QRTHTGEKPYKCPECGKSFSERS | | LREHX₁₇X₁₈X₁₉HX₂₀X₂₁X₂₂X₂₃X₂₄X₈X₉CX₁₀X₁₁CX₁₂X₁₃X₁₄X₁₅X₁₆DC | |
| | | | HLREHQRTHTGEKPYKCPECGKS | | RDLARHX₁₇X₁₈X₁₉HX₂₀X₂₁X₂₂X₂₃X₂₄X₂₅X₂₆X₂₇X₂₈X₂₉X₃₀ | |
| | | | FSDCRDLARHQRTHTGEKPTGKK | | | |
| | | | TS | | | |
| aggcacagt | AGG CAC | 1229 | LEPGEKPYKCPECGKSFSTSGNL | 1778 | X₁X₂X₃X₄X₅X₆X₇X₈X₉CX₁₀X₁₁CX₁₂X₁₃X₁₄X₁₅X₁₆TSGNLTEHX₁₇X₁₈X₁₉ | 2327 |
| gatcacagg | AGT GAT | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDHLTNHX₁₇X₁₈ | |
| cat | CAC AGG | | RSDHLTNHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆SKKALTEHX₁₇ | |
| | CAT | | GKSFSSKKALTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSGNLVRH | |
| | | | KCPECGKSFSTSGNLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆HRTTLT | |
| | | | GEKPYKCPECGKSFSHRTTLTNH | | NHX₁₇X₁₈X₁₉HX₂₀X₂₁X₂₂X₂₃X₂₄X₈X₉CX₁₀X₁₁CX₁₂X₁₃X₁₄X₁₅X₁₆SKKA | |
| | | | QRTHTGEKPYKCPECGKSFSSKK | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | ALTEHQRTHTGEKPYKCPECGKS | | DHLTNHX₁₇X₁₈X₁₉HX₂₀X₂₁X₂₂X₂₃X₂₄X₂₅X₂₆X₂₇X₂₈X₂₉X₃₀ | |
| | | | FSRSDHLTNHQRTHTGEKPTGKK | | | |
| | | | TS | | | |
| ccaagtgaa | CCA AGT | 1230 | LEPGEKPYKCPECGKSFSSKKHL | 1779 | X₁X₂X₃X₄X₅X₆X₇X₈X₉CX₁₀X₁₁CX₁₂X₁₃X₁₄X₁₅X₁₆SKKHLAEHX₁₇X₁₈X₁₉ | 2328 |
| gcccatgtg | GAA GCC | | AEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDELVRHX₁₇X₁₈ | |
| ccc | CAT gtg | | RSDELVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSGNLTEHX₁₇ | |
| | CCC | | GKSFSTSGNLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DCRDLARH | |
| | | | KCPECGKSFSDCRDLARHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QSSNLV | |
| | | | GEKPYKCPECGKSFSQSSNLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆HRTT | |
| | | | QRTHTGEKPYKCPECGKSFSHRT | | LTNHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | TLTNHQRTHTGEKPYKCPECGKS | | HSLTEHX₁₇X₁₈X₁₉HX₂₀X₂₁X₂₂X₂₃X₂₄X₂₅X₂₆X₂₇X₂₈X₂₉X₃₀ | |
| | | | FSTSHSLTEHQRTHTGEKPTGKK | | | |
| | | | TS | | | |
| gaagcccat | GAA GCC | 1231 | LEPGEKPYKCPECGKSFSSKKAL | 1780 | X₁X₂X₃X₄X₅X₆X₇X₈X₉CX₁₀X₁₁CX₁₂X₁₃X₁₄X₁₅X₁₆SKKALTEHX₁₇X₁₈X₁₉ | 2329 |
| gtgcccagg | CAT gtg | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDHLTNHX₁₇X₁₈ | |
| cac | CCC AGG | | RSDHLTNHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆SKKHLAEHX₁₇ | |
| | CAC | | GKSFSSKKHLAEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDELVRH | |
| | | | KCPECGKSFSRSDELVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSGNLT | |
| | | | GEKPYKCPECGKSFSTSGNLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆DCRD | |
| | | | QRTHTGEKPYKCPECGKSFSDCR | | LARHX₁₇X₁₈X₁₉HX₂₀X₂₁X₂₂X₂₃X₂₄X₈X₉CX₁₀X₁₁CX₁₂X₁₃X₁₄X₁₅X₁₆QS | |
| | | | DLARHQRTHTGEKPYKCPECGKS | | SNLVRHX₁₇X₁₈X₁₉HX₂₀X₂₁X₂₂X₂₃X₂₄X₂₅X₂₆X₂₇X₂₈X₂₉X₃₀ | |
| | | | FSQSSNLVRHQRTHTGEKPTGKK | | | |
| | | | TS | | | |
| agtgaagcc | AGT GAA | 1232 | LEPGEKPYKCPECGKSFSRSDHL | 1781 | X₁X₂X₃X₄X₅X₆X₇X₈X₉CX₁₀X₁₁CX₁₂X₁₃X₁₄X₁₅X₁₆RSDHLTNHX₁₇X₁₈X₁₉ | 2330 |
| catgtgccc | GCC CAT | | TNHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆SKKHLAEHX₁₇X₁₈ | |
| agg | gtg CCC | | SKKHLAEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDELVRHX₁₇ | |
| | AGG | | GKSFSRSDELVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSGNLTEH | |
| | | | KCPECGKSFSTSGNLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DCRDLA | |
| | | | GEKPYKCPECGKSFSDCRDLARH | | RHX₁₇X₁₈X₁₉HX₂₀X₂₁X₂₂X₂₃X₂₄X₈X₉CX₁₀X₁₁CX₁₂X₁₃X₁₄X₁₅X₁₆QSSN | |
| | | | QRTHTGEKPYKCPECGKSFSQSS | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆HR | |
| | | | NLVRHQRTHTGEKPYKCPECGKS | | TTLTNHX₁₇X₁₈X₁₉HX₂₀X₂₁X₂₂X₂₃X₂₄X₂₅X₂₆X₂₇X₂₈X₂₉X₃₀ | |
| | | | FSHRTTLTNHQRTHTGEKPTGKK | | | |
| | | | TS | | | |
| gtgcccagg | gtg CCC | 1233 | LEPGEKPYKCPECGKSFSSKKAL | 1782 | X₁X₂X₃X₄X₅X₆X₇X₈X₉CX₁₀X₁₁CX₁₂X₁₃X₁₄X₁₅X₁₆SKKALTEHX₁₇X₁₈X₁₉ | 2331 |
| cacagtgat | AGG CAC | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSGNLVRHX₁₇X₁₈ | |
| cac | AGT GAT | | TSGNLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆HRTTLTNHX₁₇ | |
| | CAC | | GKSFSHRTTLTNHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆SKKALTEH | |
| | | | KCPECGKSFSSKKALTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDHLT | |
| | | | GEKPYKCPECGKSFSRSDHLTNH | | NHX₁₇X₁₈X₁₉HX₂₀X₂₁X₂₂X₂₃X₂₄X₈X₉CX₁₀X₁₁CX₁₂X₁₃X₁₄X₁₅X₁₆SKKH | |
| | | | QRTHTGEKPYKCPECGKSFSSKK | | LAEHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | HLAEHQRTHTGEKPYKCPECGKS | | DELVRHX₁₇X₁₃X₁₉HX₂₀X₂₁X₂₂X₂₃X₂₄X₂₅X₂₆X₂₇X₂₈X₂₉X₃₀ | |
| | | | FSRSDELVRHQRTHTGEKPTGKK | | | |
| | | | TS | | | |
| gcccatgtg | GCC CAT | 1234 | LEPGEKPYKCPECGKSFSHRTTL | 1783 | X₁X₂X₃X₄X₅X₆X₇X₈X₉CX₁₀X₁₁CX₁₂X₁₃X₁₄X₁₅X₁₆HRTTLTNHX₁₇X₁₈X₁₉ | 2332 |
| cccaggcac | gtg CCC | | TNHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆SKKALTEHX₁₇X₁₈ | |
| agt | AGG CAC | | SKKALTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDHLTNHX₁₇ | |
| | AGT | | GKSFSRSDHLTNHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆SKKHLAEH | |
| | | | KCPECGKSFSSKKHLAEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDELV | |
| | | | GEKPYKCPECGKSFSRSDELVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆TSGN | |
| | | | QRTHTGEKPYKCPECGKSFSTSG | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆DC | |
| | | | NLTEHQRTHTGEKPYKCPECGKS | | RDLARHX₁₇X₁₈X₁₉HX₂₀X₂₁X₂₂X₂₃X₂₄X₂₅X₂₆X₂₇X₂₈X₂₉X₃₀ | |
| | | | FSDCRDLARHQRTHTGEKPTGKK | | | |
| | | | TS | | | |
| catgtgccc | CAT gtg | 1235 | LEPGEKPYKCPECGKSFSTSGNL | 1784 | X₁X₂X₃X₄X₅X₆X₇X₈X₉CX₁₀X₁₁CX₁₂X₁₃X₁₄X₁₅X₁₆TSGNLVRHX₁₇X₁₈X₁₉ | 2333 |
| aggcacagt | CCC AGG | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆HRTTLTNHX₁₇X₁₈ | |
| gat | CAC AGT | | HRTTLTNHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆SKKALTEHX₁₇ | |
| | GAT | | GKSFSSKKALTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDHLTNH | |
| | | | KCPECGKSFSRSDHLTNHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆SKKHLA | |
| | | | GEKPYKCPECGKSFSSKKHLAEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆RSDE | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | ELVRHQRTHTGEKPYKCPECGKS | | GNLTEHX₁₇X₁₈X₁₉HX₂₀X₂₁X₂₂X₂₃X₂₄X₂₅X₂₆X₂₇X₂₈X₂₉X₃₀ | |
| | | | FSTSGNLTEHQRTHTGEKPTGKK | | | |
| | | | TS | | | |
| cccaggcac | CCC AGG | 1236 | LEPGEKPYKCPECGKSFSRSDHL | 1785 | X₁X₂X₃X₄X₅X₆X₇X₈X₉CX₁₀X₁₁CX₁₂X₁₃X₁₄X₁₅X₁₆RSDHLTNHX₁₇X₁₈X₁₉ | 2334 |
| agtgatcac | CAC AGT | | TNHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆SKKALTEHX₁₇X₁₈ | |
| agg | GAT CAC | | SKKALTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSGNLVRHX₁₇ | |
| | AGG | | GKSFSTSGNLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆HRTTLTNH | |
| | | | KCPECGKSFSHRTTLTNHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆SKKALT | |
| | | | GEKPYKCPECGKSFSSKKALTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆RSDH | |
| | | | QRTHTGEKPYKCPECGKSFSRSD | | LTNHX₁₇X₁₈X₁₉HX₂₀X₂₁X₂₂X₂₃X₂₄X₈X₉CX₁₀X1CX₁₂X₁₃X₁₄X₁₅X₁₆SK | |
| | | | HLTNHQRTHTGEKPYKCPECGKS | | KHLAEHX₁₇X₁₈X₁₉HX₂₀X₂₁X₂₂X₂₃X₂₄X₂₅X₂₆X₂₇X₂₈X₂₉X₃₀ | |
| | | | ESSKKHLAEHQRTHTGEKPTGKK | | | |
| | | | TS | | | |
| gggaggcct | ggg AGG | 1237 | LEPGEKPYKCPECGKSFSTTGNL | 1786 | X₁X₂X₃X₄X₅X₆X₇X₈X₉CX₁₀X₁₁CX₁₂X₁₃X₁₄X₁₅X₁₆TTGNLTVHX₁₇X₁₈X₁₉ | 2335 |
| gcaagggcc | CCT GCA | | TVHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆DCRDLARHX₁₇X₁₈ | |
| aat | AGG GCC | | DCRDLARHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDHLTNHX₁₇ | |
| | AAT | | GKSFSRSDHLTNHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QSGDLRRH | |
| | | | KCPECGKSFSQSGDLRRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TKNSLT | |
| | | | GEKPYKCPECGKSFSTKNSLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆RSDH | |
| | | | QRTHTGEKPYKCPECGKSFSRSD | | LTNHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | HLTNHQRTHTGEKPYKCPECGKS | | DKLVRHX₁₇X₁₈X₁₉HX₂₀X₂₁X₂₂X₂₃X₂₄X₂₅X₂₆X₂₇X₂₈X₂₉X₃₀ | |
| | | | FSRSDKLVRHQRTHTGEKPTGKK | | | |
| | | | TS | | | |
| gaagggagg | GAA ggg | 1238 | LEPGEKPYKCPECGKSFSDCRDL | 1787 | X₁X₂X₃X₄X₅X₆X₇X₈X₉CX₁₀X₁₁CX₁₂X₁₃X₁₄X₁₅X₁₆DCRDLARHX₁₇X₁₈X₁₉ | 2336 |
| cctgcaagg | AGG CCT | | ARHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDHLTNHX₁₇X₁₈ | |
| gcc | GCA AGG | | RSDHLTNHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QSGDLRRHX₁₇ | |
| | GCC | | GKSFSQSGDLRRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TKNSLTEH | |
| | | | KCPECGKSFSTKNSLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDHLT | |
| | | | GEKPYKCPECGKSFSRSDHLTNH | | NHX₁₇X₁₈X₁₉HX₂₀X₂₁X₂₂X₂₃X₂₄X₈X₉CX₁₀X₁₁CX₁₂X₁₃X₁₄X₁₅X₁₆RSDK | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆QS | |
| | | | KLVRHQRTHTGEKPYKCPECGKS | | SNLVRHX₁₇X₁₈X₁₉HX₂₀X₂₁X₂₂X₂₃X₂₄X₂₅X₂₆X₂₇X₂₈X₂₉X₃₀ | |
| | | | FSQSSNLVRHQRTHTGEKPTGKK | | | |
| | | | TS | | | |
| acaggcatt | ACA GGC | 1239 | LEPGEKPYKCPECGKSFSRSDKL | 1788 | X₁X₂X₃X₄X₅X₆X₇X₈X₉CX₁₀X₁₁CX₁₂X₁₃X₁₄X₁₅X₁₆RSDKLVRHX₁₇X₁₈X₁₉ | 2337 |
| ctgggtgaa | ATT CTG | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QSSNLVRHX₁₇X₁₈ | |
| ggg | GGT GAA | | QSSNLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSGHLVRHX₁₇ | |
| | GGG | | GKSFSTSGHLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RNDALTEH | |
| | | | KCPECGKSFSRNDALTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆HKNALQ | |
| | | | GEKPYKCPECGKSFSHKNALQNH | | NHX₁₇X₁₈X₁₉HX₂₀X₂₁X₂₂X₂₃X₂₄X₈X₉CX₁₀X₁₁CX₁₂X₁₃X₁₄X₁₅X₁₆DPGH | |
| | | | QRTHTGEKPYKCPECGKSFSDPG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆SP | |
| | | | HLVRHQRTHTGEKPYKCPECGKS | | ADLTRHX₁₇X₁₈X₁₉HX₂₀X₂₁X₂₂X₂₃X₂₄X₂₅X₂₆X₂₇X₂₈X₂₉X₃₀ | |
| | | | FSSPADLTRHQRTHTGEKPTGKK | | | |
| | | | TS | | | |
| ctgggtgaa | CTG GGT | 1240 | LEPGEKPYKCPECGKSFSQSGDL | 1789 | X₁X₂X₃X₄X₅X₆X₇X₈X₉CX₁₀X₁₁CX₁₂X₁₃X₁₄X₁₅X₁₆QSGDLRRHX₁₇X₁₈X₁₉ | 2338 |
| gggaggcct | GAA ggg | | RRHQRTHTGEKPYKCPECGKSIS | | HX₂₀X₂₁X₂₂X₂₃X₂₄X₈X₉CX₁₀X₁₁CX₁₂X₁₃X₁₄X₁₅X₁₆TKNSLTEHX₁₇X₁₈ | |
| gca | AGG CCT | | TKNSLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDHLTNHX₁₇ | |
| | GCA | | GKSFSRSDHLTNHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDKLVRH | |
| | | | KCPECGKSFSRSDKLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QSSNLV | |
| | | | GEKPYKCPECGKSFSQSSNLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆TSGH | |
| | | | QRTHTGEKPYKCPECGKSFSTSG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RN | |
| | | | HLVRHQRTHTGEKPYKCPECGKS | | DALTEHX₁₇X₁₈X₁₉HX₂₀X₂₁X₂₂X₂₃X₂₄X₂₅X₂₆X₂₇X₂₈X₂₉X₃₀ | |
| | | | FSRNDALTEHQRTHTGEKPTGKK | | | |
| | | | TS | | | |
| attctgggt | ATT CTG | 1241 | LEPGEKPYKCPECGKSFSTKNSL | 1790 | X₁X₂X₃X₄X₅X₆X₇X₈X₉CX₁₀X₁₁CX₁₂X₁₃X₁₄X₁₅X₁₆TKNSLTEHX₁₇X₁₈X₁₉ | 2339 |
| gaagggagg | GGT GAA | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDHLTNHX₁₇X₁₈ | |
| cct | ggg AGG | | RSDHLTNHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDKLVRHX₁₇ | |
| | CCT | | GKSFSRSDKLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QSSNLVRH | |
| | | | KCPECGKSFSQSSNLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSGHLV | |
| | | | GEKPYKCPECGKSFSTSGHLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RNDA | |
| | | | QRTHTGEKPYKCPECGKSFSRND | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆HK | |
| | | | ALTEHQRTHTGEKPYKCPECGKS | | NALQNHX₁₇X₁₈X₁₉HX₂₀X₂₁X₂₂X₂₃X₂₄X₂₅X₂₆X₂₇X₂₈X₂₉X₃₀ | |
| | | | FSHKNALQNHQRTHTGEKPTGKK | | | |
| | | | TS | | | |
| ggcattctg | GGC ATT | 1242 | LEPGEKPYKCPECGKSFSRSDHL | 1791 | X₁X₂X₃X₄X₅X₆X₇X₈X₉CX₁₀X₁₁CX₁₂X₁₃X₁₄X₁₅X₁₆RSDHLTNHX₁₇X₁₈X₁₉ | 2340 |
| ggtgaaggg | CTG GGT | | TNHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDKLVRHX₁₇X₁₈ | |
| agg | GAA ggg | | RSDKLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QSSNLVRHX₁₇ | |
| | AGG | | GKSFSQSSNLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSGHLVRH | |
| | | | KCPECGKSFSTSGHLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RNDALT | |
| | | | GEKPYKCPECGKSFSRNDALTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆HKNA | |
| | | | QRTHTGEKPYKCPECGKSFSHKN | | LQNHX₁₇X₁₈X₁₉HX₂₀X₂₁X₂₂X₂₃X₂₄X₈X₉CX₁₀X₁₁CX₁₂XEX₁₄X₁₅X₁₆DP | |
| | | | ALQNHQRTHTGEKPYKCPECGKS | | GHLVRHX₁₇X₁₈X₁₉HX₂₀X₂₁X₂₂X₂₃X₂₄X₂₅X₂₆X₂₇X₂₈X₂₉X₃₀ | |
| | | | FSDPGHLVRHQRTHTGEKPTGKK | | | |
| | | | TS | | | |
| ggtgaaggg | GGT GAA | 1243 | LEPGEKPYKCPECGKSFSRSDHL | 1792 | X₁X₂X₃X₄X₅X₆X₇X₈X₉CX₁₀X₁₁CX₁₂X₁₃X₁₄X₁₅X₁₆RSDHLTNHX₁₇X₁₈X₁₉ | 2341 |
| aggcctgca | ggg AGG | | TNHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QSGDLRRHX₁₇X₁₈ | |
| agg | CCT GCA | | QSGDLRRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TKNSLTEHX₁₇ | |
| | AGG | | GKSFSTKNSLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDHLTNH | |
| | | | KCPECGKSFSRSDHLTNHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDKLV | |
| | | | GEKPYKCPECGKSFSRSDKLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆QSSN | |
| | | | QRTHTGEKPYKCPECGKSFSQSS | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | NLVRHQRTHTGEKPYKCPECGKS | | GHLVRHX₁₇X₁₈X₁₉HX₂₀X₂₁X₂₂X₂₃X₂₄X₂₅X₂₆X₂₇X₂₈X₂₉X₃₀ | |
| | | | FSTSGHLVRHQRTHTGEKPTGKK | | | |
| | | | TS | | | |
| ggaggcctg | GGA GGC | 1244 | LEPGEKPYKCPECGKSFSHKNAL | 1793 | X₁X₂X₃X₄X₅X₆X₇X₈X₉CX₁₀X₁₁CX₁₂X₁₃X₁₄X₁₅X₁₆HKNALQNHX₁₇X₁₈X₁₉ | 2342 |
| caagggcca | CTG CAA | | QNHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSHSLTEHX₁₇X₁₈ | |
| att | ggg CCA | | TSHSLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDKLVRHX₁₇ | |
| | ATT | | GKSFSRSDKLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QSGNLTEH | |
| | | | KCPECGKSFSQSGNLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RNDALT | |
| | | | GEKPYKCPECGKSFSRNDALTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆DPGH | |
| | | | QRTHTGEKPYKCPECGKSFSDPG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆QR | |
| | | | HLVRHQRTHTGEKPYKCPECGKS | | AHLERHX₁₇X₁₈X₁₉HX₂₀X₂₁X₂₂X₂₃X₂₄X₂₅X₂₆X₂₇X₂₈X₂₉X₃₀ | |
| | | | FSQRAHLERHQRTHTGEKPTGKK | | | |
| | | | TS | | | |
| gtgaaggga | gtg AAG | 1245 | LEPGEKPYKCPECGKSFSRSDKL | 1794 | X₁X₂X₃X₄X₅X₆X₇X₈X₉CX₁₀X₁₁CX₁₂X₁₃X₁₄X₁₅X₁₆RSDKLVRHX₁₇X₁₈X₁₉ | 2343 |
| ggcctgcaa | GGA GGC | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QSGNLTEHX₁₇X₁₈ | |
| ggg | CTG CAA | | QSGNLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RNDALTEHX₁₇ | |
| | GGG | | GKSFSRNDALTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DPGHLVRH | |
| | | | KCPECGKSFSDPGHLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QRAHLE | |
| | | | GEKPYKCPECGKSFSQRAHLERH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RKDN | |
| | | | QRTHTGEKPYKCPECGKSFSRKD | | LKNHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | NLKNHQRTHTGEKPYKCPECGKS | | DELVRHX₁₇X₁₈X₁₉HX₂₀X₂₁X₂₂X₂₃X₂₄X₂₅X₂₆X₂₇X₂₈X₂₉X₃₀ | |
| | | | FSRSDELVRHQRTHTGEKPTGKK | | | |
| | | | TS | | | |
| tgggtgaag | TGG gtg | 1246 | LEPGEKPYKCPECGKSFSQSGNL | 1795 | X₁X₂X₃X₄X₅X₆X₇X₈X₉CX₁₀X₁₁CX₁₂X₁₃X₁₄X₁₅X₁₆QSGNLTEHX₁₇X₁₈X₁₉ | 2344 |
| ggaggcctg | AAG GGA | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RNDALTEHX₁₇X₁₈ | |
| caa | GGC CTG | | RNDALTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DPGHLVRHX₁₇ | |
| | CAA | | GKSFSDPGHLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QRAHLERH | |
| | | | KCPECGKSFSQRAHLERHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RKDNLK | |
| | | | GEKPYKCPECGKSFSRKDNLKNH | | NHX₁₇X₁₈X₁₉HX₂₀X₂₁X₂₂X₂₃X₂₄X₈X₉CX₁₀X₁₁CX₁₂X₁₃X₁₄X₁₅X₁₆RSDE | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | ELVRHQRTHTGEKPYKCPECGKS | | DHLTTHX₁₇X₁₈X₁₉HX₂₀X₂₁X₂₂X₂₃X₂₄X₂₅X₂₆X₂₇X₂₈X₂₉X₃₀ | |
| | | | FSRSDHLTTHQRTHTGEKPTGKK | | | |
| | | | TS | | | |
| aagggaggc | AAG GGA | 1247 | LEPGEKPYKCPECGKSFSTSHSL | 1796 | X₁X₂X₃X₄X₅X₆X₇X₈X₉CX₁₀X₁₁CX₁₂X₁₃X₁₄X₁₅X₁₆TSHSLTEHX₁₇X₁₈X₁₉ | 2345 |
| ctgcaaggg | GGC CTG | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDKLVRHX₁₇X₁₈ | |
| cca | CAA ggg | | RSDKLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QSGNLTEHX₁₇ | |
| | CCA | | GKSFSQSGNLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RNDALTEH | |
| | | | KCPECGKSFSRNDALTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DPGHLV | |
| | | | GEKPYKCPECGKSFSDPGHLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆QRAH | |
| | | | QRTHTGEKPYKCPECGKSFSQRA | | LERHX₁₇X₁₈X₁₉HX₂₀X₂₁X₂₂X₂₃X₂₄X₈X₉CX₁₀X₁₁CX₁₂X₁₃X₁₄X₁₅X₁₆RK | |
| | | | HLERHQRTHTGEKPYKCPECGKS | | DNLKNHX₁₇X₁₈X₁₉HX₂₀X₂₁X₂₂X₂₃X₂₄X₂₅X₂₆X₂₇X₂₈X₂₉X₃₀ | |
| | | | FSRKDNLKNHQRTHTGEKPTGKK | | | |
| | | | TS | | | |
| actgtgcct | ACT gtg | 1248 | LEPGEKPYKCPECGKSFSDPGHL | 1797 | X₁X₂X₃X₄X₅X₆X₇X₈X₉CX₁₀X₁₁CX₁₂X₁₃X₁₄X₁₅X₁₆DPGHLVRHX₁₇X₁₈X₁₉ | 2346 |
| gggcacatg | CCT ggg | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RRDELNVHX₁₇X₁₈ | |
| ggc | CAC ATG | | RRDELNVHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆SKKALTEHX₁₇ | |
| | GGC | | GKSFSSKKALTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDKLVRH | |
| | | | KCPECGKSFSRSDKLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TKNSLT | |
| | | | GEKPYKCPECGKSFSTKNSLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆RSDE | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆TH | |
| | | | ELVRHQRTHTGEKPYKCPECGKS | | LDLIRHX₁₇X₁₈X₁₉HX₂₀X₂₁X₂₂X₂₃X₂₄X₂₅X₂₆X₂₇X₂₈X₂₉X₃₀ | |
| | | | FSTHLDLIRHQRTHTGEKPTGKK | | | |
| | | | TS | | | |
| gcctgggca | GCC TGG | 1249 | LEPGEKPYKCPECGKSFSSKKAL | 1798 | X₁X₂X₃X₄X₅X₆X₇X₈X₉CX₁₀X₁₁CX₁₂X₁₃X₁₄X₁₅X₁₆SKKALTEHX₁₇X₁₈X₁₉ | 2347 |
| catgggctt | GCA CAT | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TTGALTEHX₁₇X₁₈ | |
| cac | ggg CTT | | TTGALTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDKLVRHX₁₇ | |
| | CAC | | GKSFSRSDKLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSGNLTEH | |
| | | | KCPECGKSFSTSGNLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QSGDLR | |
| | | | GEKPYKCPECGKSFSQSGDLRRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDH | |
| | | | QRTHTGEKPYKCPECGKSFSRSD | | LTTHX₁₇X₁₈X₁₉HX₂₀X₂₁X₂₂X₂₃X₂₄X₈X₉CX₁₀X₁₁CX₁₂X₁₃X₁₄X₁₅X₁₆DC | |
| | | | HLTTHQRTHTGEKPYKCPECGKS | | RDLARHX₁₇X₁₈X₁₉HX₂₀X₂₁X₂₂X₂₃X₂₄X₂₅X₂₆X₂₇X₂₈X₂₉X₃₀ | |
| | | | FSDCRDLARHQRTHTGEKPTGKK | | | |
| | | | TS | | | |
| gctggcctg | GCT GGC | 1250 | LEPGEKPYKCPECGKSFSTKNSL | 1799 | X₁X₂X₃X₄X₅X₆X₇X₈X₉CX₁₀X₁₁CX₁₂X₁₃X₁₄X₁₅X₁₆TKNSLTEHX₁₇X₁₈X₁₉ | 2348 |
| taggaccaa | CTG TAG | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QSGNLTEHX₁₇X₁₈ | |
| cct | GAC CAA | | QSGNLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DPGNLVRHX₁₇ | |
| | CCT | | GKSFSDPGNLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆REDNLHTH | |
| | | | KCPECGKSFSREDNLHTHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RNDALT | |
| | | | GEKPYKCPECGKSFSRNDALTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆DPGH | |
| | | | QRTHTGEKPYKCPECGKSFSDPG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | HLVRHQRTHTGEKPYKCPECGKS | | GELVRHX₁₇X₁₈X₁₉HX₂₀X₂₁X₂₂X₂₃X₂₄X₂₅X₂₆X₂₇X₂₈X₂₉X₃₀ | |
| | | | FSTSGELVRHQRTHTGEKPTGKK | | | |
| | | | TS | | | |
| ggcctgtag | GGC CTG | 1251 | LEPGEKPYKCPECGKSFSDKKDL | 1800 | X₁X₂X₃X₄X₅X₆X₇X₈X₉CX₁₀X₁₁CX₁₂X₁₃X₁₄X₁₅X₁₆DKKDLTRHX₁₇X₁₈X₁₉ | 2349 |
| gaccaacct | TAG GAC | | TRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TKNSLTEHX₁₇X₁₈ | |
| acc | CAA CCT | | TKNSLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QSGNLTEHX₁₇ | |
| | ACC | | GKSFSQSGNLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DPGNLVRH | |
| | | | KCPECGKSFSDPGNLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆REDNLH | |
| | | | GEKPYKCPECGKSFSREDNLHTH | | THX₁₇X₁₈X₁₉HX₂₀X₂₁X₂₂X₂₃X₂₄X₈X₉CX₁₀X₁₁CX₁₂X₁₃X₁₄X₁₅X₁₆RNDA | |
| | | | QRTHTGEKPYKCPECGKSFSRND | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆DP | |
| | | | ALTEHQRTHTGEKPYKCPECGKS | | GHLVRHX₁₇X₁₈X₁₉HX₂₀X₂₁X₂₂X₂₃X₂₄X₂₅X₂₆X₂₇X₂₈X₂₉X₃₀ | |
| | | | FSDPGHLVRHQRTHTGEKPTGKK | | | |
| | | | TS | | | |
| ctgtaggac | CTG TAG | 1252 | LEPGEKPYKCPECGKSFSHKNAL | 1801 | X₁X₂X₃X₄X₅X₆X₇X₈X₉CX₁₀X₁₁CX₁₂X₁₃X₁₄X₁₅X₁₆HKNALQNHX₁₇X₁₈X₁₉ | 2350 |
| caacctacc | GAC CAA | | QNHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆DKKDLTRHX₁₇X₁₈ | |
| att | CCT ACC | | DKKDLTRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TKNSLTEHX₁₇ | |
| | ATT | | GKSFSTKNSLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QSGNLTEH | |
| | | | KCPECGKSFSQSGNLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DPGNLV | |
| | | | GEKPYKCPECGKSFSDPGNLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆REDN | |
| | | | QRTHTGEKPYKCPECGKSFSRED | | LHTHX₁₇X₁₈X₁₉HX₂₀X₂₁X₂₂X₂₃X₂₄X₈X₉CX₁₀X₁₁CX₁₂X₁₃X₁₄X₁₅X₁₆RN | |
| | | | NLHTHQRTHTGEKPYKCPECGKS | | DALTEHX₁₇X₁₈X₁₉HX₂₀X₂₁X₂₂X₂₃X₂₄X₂₅X₂₆X₂₇X₂₈X₂₉X₃₀ | |
| | | | FSRNDALTEHQRTHTGEKPTGKK | | | |
| | | | TS | | | |
| ccaccccaa | CCA CCC | 1253 | LEPGEKPYKCPECGKSFSTSHSL | 1802 | X₁X₂X₃X₄X₅X₆X₇X₈X₉CX₁₀X₁₁CX₁₂X₁₃X₁₄X₁₅X₁₆TSHSLTEHX₁₇X₁₈X₁₉ | 2351 |
| agttgagtg | CAA AGT | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDELVRHX₁₇X₁₈ | |
| cca | TGA gtg | | RSDELVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QAGHLASHX₁₇ | |
| | CCA | | GKSFSQAGHLASHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆HRTTLTNH | |
| | | | KCPECGKSFSHRTTLTNHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QSGNLT | |
| | | | GEKPYKCPECGKSFSQSGNLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆SKKH | |
| | | | QRTHTGEKPYKCPECGKSFSSKK | | LAEHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | HLAEHQRTHTGEKPYKCPECGKS | | HSLTEHX₁₇X₁₈X₁₉HX₂₀X₂₁X₂₂X₂₃X₂₄X₂₅X₂₆X₂₇X₂₈X₂₉X₃₀ | |
| | | | FSTSHSLTEHQRTHTGEKPTGKK | | | |
| | | | TS | | | |
| ccccaaagt | CCC CAA | 1254 | LEPGEKPYKCPECGKSFSRKDNL | 1803 | X₁X₂X₃X₄X₅X₆X₇X₈X₉CX₁₀X₁₁CX₁₂X₁₃X₁₄X₁₅X₁₆RKDNLKNHX₁₇X₁₈X₁₉ | 2352 |
| tgagtgcca | AGT TGA | | KNHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSHSLTEHX₁₇X₁₈ | |
| aag | gtg CCA | | TSHSLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDELVRHX₁₇ | |
| | AAG | | GKSFSRSDELVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QAGHLASH | |
| | | | KCPECGKSFSQAGHLASHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆HRTTLT | |
| | | | GEKPYKCPECGKSFSHRTTLTNH | | NHX₁₇X₁₈X₁₉HX₂₀X₂₁X₂₂X₂₃X₂₄X₈X₉CX₁₀X₁₁CX₁₂X₁₃X₁₄X₁₅X₁₆QSGN | |
| | | | QRTHTGEKPYKCPECGKSFSQSG | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆SK | |
| | | | NLTEHQRTHTGEKPYKCPECGKS | | KHLAEHX₁₇X₁₈X₁₉HX₂₀X₂₁X₂₂X₂₃X₂₄X₂₅X₂₆X₂₇X₂₈X₂₉X₃₀ | |
| | | | FSSKKHLAEHQRTHTGEKPTGKK | | | |
| | | | TS | | | |
| gctcctgga | GCT CCT | 1255 | LEPGEKPYKCPECGKSFSDKKDL | 1804 | X₁X₂X₃X₄X₅X₆X₇X₈X₉CX₁₀X₁₁CX₁₂X₁₃X₁₄X₁₅X₁₆DKKDLTRHX₁₇X₁₈X₁₉ | 2353 |
| cccaggcac | GGA CCC | | TRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆SKKALTEHX₁₇X₁₈ | |
| acc | AGG CAC | | SKKALTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDHLTNHX₁₇ | |
| | ACC | | GKSFSRSDHLTNHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆SKKHLAEH | |
| | | | KCPECGKSFSSKKHLAEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QRAHLE | |
| | | | GEKPYKCPECGKSFSQRAHLERH | | RHX₁₇X₁₈X₁₉HX₂₀X₂₁X₂₂X₂₃X₂₄X₈X₉CX₁₀X₁₁CX₁₂X₁₃X₁₄X₁₅X₁₆TKNS | |
| | | | QRTHTGEKPYKCPECGKSFSTKN | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | SLTEHQRTHTGEKPYKCPECGKS | | GELVRHX₁₇X₁₈X₁₉HX₂₀X₂₁X₂₂X₂₃X₂₄X₂₅X₂₆X₂₇X₂₈X₂₉X₃₀ | |
| | | | FSTSGELVRHQRTHTGEKPTGKK | | | |
| | | | TS | | | |
| ctggaccca | CTG GAC | 1256 | LEPGEKPYKCPECGKSFSDCRDL | 1805 | X₁X₂X₃X₄X₅X₆X₇X₈X₉CX₁₀X₁₁CX₁₂X₁₃X₁₄X₁₅X₁₆DCRDLARHX₁₇X₁₈X₁₉ | 2354 |
| ggcacacct | CCA GGC | | ARHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TKNSLTEHX₁₇X₁₈ | |
| gcc | ACA CCT | | TKNSLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆SPADLTRHX₁₇ | |
| | GCC | | GKSFSSPADLTRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DPGHLVRH | |
| | | | KCPECGKSFSDPGHLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSHSLT | |
| | | | GEKPYKCPECGKSFSTSHSLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆DPGN | |
| | | | QRTHTGEKPYKCPECGKSFSDPG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RN | |
| | | | NLVRHQRTHTGEKPYKCPECGKS | | DALTEHX₁₇X₁₈X₁₉HX₂₀X₂₁X₂₂X₂₃X₂₄X₂₅X₂₆X₂₇X₂₈X₂₉X₃₀ | |
| | | | FSRNDALTEHQRTHTGEKPTGKK | | | |
| | | | TS | | | |
| gcccccact | GCC CCC | 1257 | LEPGEKPYKCPECGKSFSRSDKL | 1806 | X₁X₂X₃X₄X₅X₆X₇X₈X₉CX₁₀X₁₁CX₁₂X₁₃X₁₄X₁₅X₁₆RSDKLVRHX₁₇X₁₈X₁₉ | 2355 |
| ggcacacct | ACT GGC | | VRHQRTHTGEKPYKCPECGKSFS | | HX₂₀X20X₂₂X₂₃X₂₄X₈X₉CX₁₀X₁₁CX₁₂X₁₃X₁₄X₁₅X₁₆TKNSLTEHX₁₇X₁₈ | |
| ggg | ACA CCT | | TKNSLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆SPADLTRHX₁₇ | |
| | GGG | | GKSFSSPADLTRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DPGHLVRH | |
| | | | KCPECGKSFSDPGHLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆THLDL | |
| | | | GEKPYKCPECGKSFSTHLDLIRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆SKKH | |
| | | | QRTHTGEKPYKCPECGKSFSSKK | | LAEHX₁₇X₁₈X₁₉HX₂₀X₂₁X₂₂X₂₃X₂₄X₈X₉CX₁₀X₁₁CX₁₂X₁₃X₁₄X₁₅X₁₆DC | |
| | | | HLAEHQRTHTGEKPYKCPECGKS | | RDLARHX₁₇X₁₈X₁₉HX₂₀X₂₁X₂₂X₂₃X₂₄X₂₅X₂₆X₂₇X₂₈X₂₉X₃₀ | |
| | | | FSDCRDLARHQRTHTGEKPTGKK | | | |
| | | | TS | | | |
| cctgccccc | CCT GCC | 1258 | LEPGEKPYKCPECGKSFSTKNSL | 1807 | X₁X₂X₃X₄X₅X₆X₇X₈X₉CX₁₀X₁₁CX₁₂X₁₃X₁₄X₁₅X₁₆TKNSLTEHX₁₇X₁₈X₁₉ | 2356 |
| actggcaca | CCC ACT | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆SPADLTRHX₁₇X₁₈ | |
| cct | GGC ACA | | SPADLTRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DPGHLVRHX₁₇ | |
| | CCT | | GKSFSDPGHLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆THLDLIRH | |
| | | | KCPECGKSFSTHLDLIRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆SKKHLA | |
| | | | GEKPYKCPECGKSFSSKKHLAEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆DCRD | |
| | | | QRTHTGEKPYKCPECGKSFSDCR | | LARHX₁₇X₁₈X₁₉HX₂₀X₂₁X₂₂X₂₃X₂₄X₈X₉CX₁₀X₁₁CX₁₂X₁₃X₁₄X₁₅X₁₆TK | |
| | | | DLARHQRTHTGEKPYKCPECGKS | | NSLTEHX₁₇X₁₃X₁₉HX₂₀X₂₁X₂₂X₂₃X₂₄X₂₅X₂₆X₂₇X₂₈X₂₉X₃₀ | |
| | | | FSTKNSLTEHQRTHTGEKPTGKK | | | |
| | | | TS | | | |
| ggcacacct | GGC ACA | 1259 | LEPGEKPYKCPECGKSFSDPGHL | 1808 | X₁X₂X₃X₄X₅X₆X₇X₈X₉CX₁₀X₁₁CX₁₂X₁₃X₁₄X₁₅X₁₆DPGHLVRHX₁₇X₁₈X₁₉ | 2357 |
| gcccccact | CCT GCC | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆THLDLIRHX₁₇X₁₈ | |
| ggc | CCC ACT | | THLDLIRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆SKKHLAEHX₁₇ | |
| | GGC | | GKSFSSKKHLAEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DCRDLARH | |
| | | | KCPECGKSFSDCRDLARHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TKNSLT | |
| | | | GEKPYKCPECGKSFSTKNSLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆SPAD | |
| | | | QRTHTGEKPYKCPECGKSFSSPA | | LTRHX₁₇X₁₈X₁₉HX₂₀X₂₁X₂₂X₂₃X₂₄X₈X₉CX₁₀X₁₁CX₁₂X₁₃X₁₄X₁₅X₁₆DP | |
| | | | DLTRHQRTHTGEKPYKCPECGKS | | GHLVRHX₁₇X₁₈X₁₉HX₂₀X₂₁X₂₂X₂₃X₂₄X₂₅X₂₆X₂₇X₂₈X₂₉X₃₀ | |
| | | | FSDPGHLVRHQRTHTGEKPTGKK | | | |
| | | | TS | | | |
| acacctgcc | ACA CCT | 1260 | LEPGEKPYKCPECGKSFSSPADL | 1809 | X₁X₂X₃X₄X₅X₆X₇X₈X₉CX₁₀X₁₁CX₁₂X₁₃X₁₄X₁₅X₁₆SPADLTRHX₁₇X₁₈X₁₉ | 2358 |
| cccactggc | GCC CCC | | TRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆DPGHLVRHX₁₇X₁₈ | |
| aca | ACT GGC | | DPGHLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆THLDLIRHX₁₇ | |
| | ACA | | GKSFSTHLDLIRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆SKKHLAEH | |
| | | | KCPECGKSFSSKKHLAEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DCRDLA | |
| | | | GEKPYKCPECGKSFSDCRDLARH | | RHX₁₇X₁₈X₁₉HX₂₀X₂₁X₂₂X₂₃X₂₄X₈X₉CX₁₀X₁₁CX₁₂X₁₃X₁₄X₁₅X₁₆TKNS | |
| | | | QRTHTGEKPYKCPECGKSFSTKN | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆SP | |
| | | | SLTEHQRTHTGEKPYKCPECGKS | | ADLTRHX₁₇X₁₈X₁₉HX₂₀X₂₁X₂₂X₂₃X₂₄X₂₅X₂₆X₂₇X₂₈X₂₉X₃₀ | |
| | | | FSSPADLTRHQRTHTGEKPTGKK | | | |
| | | | TS | | | |
| ccaggcaca | CCA GGC | 1261 | LEPGEKPYKCPECGKSFSTHLDL | 1810 | X₁X₂X₃X₄X₅X₆X₇X₈X₉CX₁₀X₁₁CX₁₂X₁₃X₁₄X₁₅X₁₆THLDLIRHX₁₇X₁₈X₁₉ | 2359 |
| cctgccccc | ACA CCT | | IRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆SKKHLAEHX₁₇X₁₈ | |
| act | GCC CCC | | SKKHLAEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DCRDLARHX₁₇ | |
| | ACT | | GKSFSDCRDLARHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TKNSLTEH | |
| | | | KCPECGKSFSTKNSLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆SPADLT | |
| | | | GEKPYKCPECGKSFSSPADLTRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆DPGH | |
| | | | QRTHTGEKPYKCPECGKSFSDPG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | HLVRHQRTHTGEKPYKCPECGKS | | HSLTEHX₁₇X₁₈X₁₉HX₂₀X₂₁X₂₂X₂₃X₂₄X₂₅X₂₆X₂₇X₂₈X₂₉X₃₀ | |
| | | | FSTSHSLTEHQRTHTGEKPTGKK | | | |
| | | | TS | | | |
| cccactggc | CCC ACT | 1262 | LEPGEKPYKCPECGKSFSSKKAL | 1811 | X₁X₂X₃X₄X₅X₆X₇X₈X₉CX₁₀X₁₁CX₁₂X₁₃X₁₄X₁₅X₁₆SKKALTEHX₁₇X₁₈X₁₉ | 2360 |
| acacctggg | GGC ACA | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDKLVRHX₁₇X₁₈ | |
| cac | CCT ggg | | RSDKLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TKNSLTEHX₁₇ | |
| | CAC | | GKSFSTKNSLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆SPADLTRH | |
| | | | KCPECGKSFSSPADLTRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DPGHLV | |
| | | | GEKPYKCPECGKSFSDPGHLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆THLD | |
| | | | QRTHTGEKPYKCPECGKSFSTHL | | LIRHX₁₇X₁₈X₁₉HX₂₀X₂₁X₂₂X₂₃X₂₄X₈X₉CX₁₀X₁₁CX₁₂X₁₃X₁₄X₁₅X₁₆SK | |
| | | | DLIRHQRTHTGEKPYKCPECGKS | | KHLAEHX₁₇X₁₈X₁₉HX₂₀X₂₁X₂₂X₂₃X₂₄X₂₅X₂₆X₂₇X₂₈X₂₉X₃₀ | |
| | | | FSSKKHLAEHQRTHTGEKPTGKK | | | |
| | | | TS | | | |
| gacccaggc | GAC CCA | 1263 | LEPGEKPYKCPECGKSFSSKKHL | 1812 | X₁X₂X₃X₄X₅X₆X₇X₈X₉CX₁₀X₁₁CX₁₂X₁₃X₁₄X₁₅X₁₆SKKHLAEHX₁₇X₁₈X₁₉ | 2361 |
| acacctgcc | GGC ACA | | AEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆DCRDLARHX₁₇X₁₈ | |
| ccc | CCT GCC | | DCRDLARHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TKNSLTEHX₁₇ | |
| | CCC | | GKSFSTKNSLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆SPADLTRH | |
| | | | KCPECGKSFSSPADLTRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DPGHLV | |
| | | | GEKPYKCPECGKSFSDPGHLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆TSHS | |
| | | | QRTHTGEKPYKCPECGKSFSTSH | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆DP | |
| | | | SLTEHQRTHTGEKPYKCPECGKS | | GNLVRHX₁₇X₁₈X₁₉HX₂₀X₂₁X₂₂X₂₃X₂₄X₂₅X₂₆X₂₇X₂₈X₂₉X₃₀ | |
| | | | FSDPGNLVRHQRTHTGEKPTGKK | | | |
| | | | TS | | | |
| ccactggca | CCA CTG | 1264 | LEPGEKPYKCPECGKSFSSPADL | 1813 | X₁X₂X₃X₄X₅X₆X₇X₈X₉CX₁₀X₁₁CX₁₂X₁₃X₁₄X₁₅X₁₆SPADLTRHX₁₇X₁₈X₁₉ | 2362 |
| cacctgggc | GCA CAC | | TRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆DPGHLVRHX₁₇X₁₈ | |
| aca | CTG GGC | | DPGHLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RNDALTEHX₁₇ | |
| | ACA | | GKSFSRNDALTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆SKKALTEH | |
| | | | KCPECGKSFSSKKALTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QSGDLR | |
| | | | GEKPYKCPECGKSFSQSGDLRRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RNDA | |
| | | | QRTHTGEKPYKCPECGKSFSRND | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | ALTEHQRTHTGEKPYKCPECGKS | | HSLTEHX₁₇X₁₈X₁₉HX₂₀X₂₁X₂₂X₂₃X₂₄X₂₅X₂₆X₂₇X₂₈X₂₉X₃₀ | |
| | | | FSTSHSLTEHQRTHTGEKPTGKK | | | |
| | | | TS | | | |
| gcacacctg | GCA CAC | 1265 | LEPGEKPYKCPECGKSFSQSGDL | 1814 | X₁X₂X₃X₄X₅X₆X₇X₈X₉CX₁₀X₁₁CX₁₂X₁₃X₁₄X₁₅X₁₆QSGDLRRHX₁₇X₁₈X₁₉ | 2363 |
| cccccactg | CTG CCC | | RRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RNDALTEHX₁₇X₁₈ | |
| gca | CCA CTG | | RNDALTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSHSLTEHX₁₇ | |
| | GCA | | GKSFSTSHSLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆SKKHLAEH | |
| | | | KCPECGKSFSSKKHLAEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RNDALT | |
| | | | GEKPYKCPECGKSFSRNDALTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆SKKA | |
| | | | QRTHTGEKPYKCPECGKSFSSKK | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆QS | |
| | | | ALTEHQRTHTGEKPYKCPECGKS | | GDLRRHX₁₇X₁₈X₁₉HX₂₀X₂₁X₂₂X₂₃X₂₄X₂₅X₂₆X₂₇X₂₈X₂₉X₃₀ | |
| | | | FSQSGDLRRHQRTHTGEKPTGKK | | | |
| | | | TS | | | |
| ctgccccca | CTG CCC | 1266 | LEPGEKPYKCPECGKSFSRNDAL | 1815 | X₁X₂X₃X₄X₅X₆X₇X₈X₉CX₁₀X₁₁CX₁₂X₁₃X₁₄X₁₅X₁₆RNDALTEHX₁₇X₁₈X₁₉ | 2364 |
| ctggcacac | CCA CTG | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆SKKALTEHX₁₇X₁₈ | |
| ctg | GCA CAC | | SKKALTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QSGDLRRHX₁₇ | |
| | CTG | | GKSFSQSGDLRRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RNDALTEH | |
| | | | KCPECGKSFSRNDALTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSHSLT | |
| | | | GEKPYKCPECGKSFSTSHSLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆SKKH | |
| | | | QRTHTGEKPYKCPECGKSFSSKK | | LAEHX₁₇X₁₈X₁₉HX₂₀X₂₁X₂₂X₂₃X₂₄X₈X₉CX₁₀X₁₁CX₁₂X₁₃X₁₄X₁₅X₁₆RN | |
| | | | HLAEHQRTHTGEKPYKCPECGKS | | DALTEHX₁₇X₁₈X₁₉HX₂₀X₂₁X₂₂X₂₃X₂₄X₂₅X₂₆X₂₇X₂₈X₂₉X₃₀ | |
| | | | FSRNDALTEHQRTHTGEKPTGKK | | | |
| | | | TS | | | |
| cccccactg | CCC CCA | 1267 | LEPGEKPYKCPECGKSFSDPGHL | 1816 | X₁X₂X₃X₄X₅X₆X₇X₈X₉CX₁₀X₁₁CX₁₂X₁₃X₁₄X₁₅X₁₆DPGHLVRHX₁₇X₁₈X₁₉ | 2365 |
| gcacacctg | CTG GCA | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RNDALTEHX₁₇X₁₈ | |
| ggc | CAC CTG | | RNDALTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆SKKALTEHX₁₇ | |
| | GGC | | GKSFSSKKALTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QSGDLRRH | |
| | | | KCPECGKSFSQSGDLRRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RNDALT | |
| | | | GEKPYKCPECGKSFSRNDALTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆TSHS | |
| | | | QRTHTGEKPYKCPECGKSFSTSH | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆SK | |
| | | | SLTEHQRTHTGEKPYKCPECGKS | | KHLAEHX₁₇X₁₈X₁₉HX₂₀X₂₁X₂₂X₂₃X₂₄X₂₅X₂₆X₂₇X₂₈X₂₉X₃₀ | |
| | | | FSSKKHLAEHQRTHTGEKPTGKK | | | |
| | | | TS | | | |
| caggcacac | CAG GCA | 1268 | LEPGEKPYKCPECGKSFSRNDAL | 1817 | X₁X₂X₃X₄X₅X₆X₇X₈X₉CX₁₀X₁₁CX₁₂X₁₃X₁₄X₁₅X₁₆RNDALTEHX₁₇X₁₈X₁₉ | 2366 |
| ctgccccca | CAC CTG | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSHSLTEHX₁₇X₁₈ | |
| ctg | CCC CCA | | TSHSLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆SKKHLAEHX₁₇ | |
| | CTG | | GKSFSSKKHLAEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RNDALTEH | |
| | | | KCPECGKSFSRNDALTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆SKKALT | |
| | | | GEKPYKCPECGKSFSSKKALTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆QSGD | |
| | | | QRTHTGEKPYKCPECGKSFSQSG | | LRRHX₁₇X₁₈X₁₉HX₂₀X₂₁X₂₂X₂₃X₂₄X₈X₉CX₁₀X₁₁CX₁₂X₁₃X₁₄X₁₅X₁₆RA | |
| | | | DLRRHQRTHTGEKPYKCPECGKS | | DNLTEHX₁₇X₁₈X₁₉HX₂₀X₂₁X₂₂X₂₃X₂₄X₂₅X₂₆X₂₇X₂₈X₂₉X₃₀ | |
| | | | FSRADNLTEHQRTHTGEKPTGKK | | | |
| | | | TS | | | |
| tggacccag | TGG ACC | 1269 | LEPGEKPYKCPECGKSFSSKKHL | 1818 | X₁X₂X₃X₄X₅X₆X₇X₈X₉CX₁₀X₁₁CX₁₂X₁₃X₁₄X₁₅X₁₆SKKHLAEHX₁₇X₁₈X₁₉ | 2367 |
| gcacacctg | CAG GCA | | AEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RNDALTEHX₁₇X₁₈ | |
| ccc | CAC CTG | | RNDALTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆SKKALTEHX₁₇ | |
| | CCC | | GKSFSSKKALTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QSGDLRRH | |
| | | | KCPECGKSFSQSGDLRRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RADNLT | |
| | | | GEKPYKCPECGKSFSRADNLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆DKKD | |
| | | | QRTHTGEKPYKCPECGKSFSDKK | | LTRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | DLTRHQRTHTGEKPYKCPECGKS | | DHLTTHX₁₇X₁₈X₁₉HX₂₀X₂₁X₂₂X₂₃X₂₄X₂₅X₂₆X₂₇X₂₈X₂₉X₃₀ | |
| | | | FSRSDHLTTHQRTHTGEKPTGKK | | | |
| | | | TS | | | |
| acccaggca | ACC CAG | 1270 | LEPGEKPYKCPECGKSFSTSHSL | 1819 | X₁X₂X₃X₄X₅X₆X₇X₈X₉CX₁₀X₁₁CX₁₂X₁₃X₁₄X₁₅X₁₆TSHSLTEHX₁₇X₁₈X₁₉ | 2368 |
| cacctgccc | GCA CAC | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆SKKHLAEHX₁₇X₁₈ | |
| cca | CTG CCC | | SKKHLAEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RNDALTEHX₁₇ | |
| | CCA | | GKSFSRNDALTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆SKKALTEH | |
| | | | KCPECGKSFSSKKALTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QSGDLR | |
| | | | GEKPYKCPECGKSFSQSGDLRRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RADN | |
| | | | QRTHTGEKPYKCPECGKSFSRAD | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆DK | |
| | | | NLTEHQRTHTGEKPYKCPECGKS | | KDLTRHX₁₇X₁₈X₁₉HX₂₀X₂₁X₂₂X₂₃X₂₄X₂₅X₂₆X₂₇X₂₈X₂₉X₃₀ | |
| | | | FSDKKDLTRHQRTHTGEKPTGKK | | | |
| | | | TS | | | |
| cacctgccc | CAC CTG | 1271 | LEPGEKPYKCPECGKSFSSKKAL | 1820 | X₁X₂X₃X₄X₅X₆X₇X₈X₉CX₁₀X₁₁CX₁₂X₁₃X₁₄X₁₅X₁₆SKKALTEHX₁₇X₁₈X₁₉ | 2369 |
| ccactggca | CCC CCA | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QSGDLRRHX₁₇X₁₈ | |
| cac | CTG GCA | | QSGDLRRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RNDALTEHX₁₇ | |
| | CAC | | GKSFSRNDALTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSHSLTEH | |
| | | | KCPECGKSFSTSHSLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆SKKHLA | |
| | | | GEKPYKCPECGKSFSSKKHLAEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆RNDA | |
| | | | QRTHTGEKPYKCPECGKSFSRND | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆SK | |
| | | | ALTEHQRTHTGEKPYKCPECGKS | | KALTEHX₁₇X₁₈X₁₉HX₂₀X₂₁X₂₂X₂₃X₂₄X₂₅X₂₆X₂₇X₂₈X₂₉X₃₀ | |
| | | | FSSKKALTEHQRTHTGEKPTGKK | | | |
| | | | TS | | | |
| ccacctacc | CCA CCT | 1272 | LEPGEKPYKCPECGKSFSSRRTC | 1821 | X₁X₂X₃X₄X₅X₆X₇X₈X₉CX₁₀X₁₁CX₁₂X₁₃X₁₄X₁₅X₁₆SRRTCRAHX₁₇X₁₈X₁₉ | 2370 |
| attgcccat | ACC ATT | | RAHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSGNLTEHX₁₇X₁₈ | |
| cgt | GCC CAT | | TSGNLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DCRDLARHX₁₇ | |
| | CGT | | GKSFSDCRDLARHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆HKNALQNH | |
| | | | KCPECGKSFSHKNALQNHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DKKDLT | |
| | | | GEKPYKCPECGKSFSDKKDLTRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆TKNS | |
| | | | QRTHTGEKPYKCPECGKSFSTKN | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | SLTEHQRTHTGEKPYKCPECGKS | | HSLTEHX₁₇X₁₈X₁₉HX₂₀X₂₁X₂₂X₂₃X₂₄X₂₅X₂₆X₂₇X₂₈X₂₉X₃₀ | |
| | | | FSTSHSLTEHQRTHTGEKPTGKK | | | |
| | | | TS | | | |
| tggcacacc | TGG CAC | 1273 | LEPGEKPYKCPECGKSFSRNDAL | 1822 | X₁X₂X₃X₄X₅X₆X₇X₈X₉CX₁₀X₁₁CX₁₂X₁₃X₁₄X₁₅X₁₆RNDALTEHX₁₇X₁₈X₁₉ | 2371 |
| tgggcacat | ACC TGG | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSGNLTEHX₁₇X₁₈ | |
| ctg | GCA CAT | | TSGNLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QSGDLRRHX₁₇ | |
| | CTG | | GKSFSQSGDLRRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDHLTTH | |
| | | | KCPECGKSFSRSDHLTTHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DKKDLT | |
| | | | GEKPYKCPECGKSFSDKKDLTRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆SKKA | |
| | | | QRTHTGEKPYKCPECGKSFSSKK | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | ALTEHQRTHTGEKPYKCPECGKS | | DHLTTHX₁₇X₁₈X₁₉HX₂₀X₂₁X₂₂X₂₃X₂₄X₂₅X₂₆X₂₇X₂₈X₂₉X₃₀ | |
| | | | FSRSDHLTTHQRTHTGEKPTGKK | | | |
| | | | TS | | | |
| cactggcac | CAC TGG | 1274 | LEPGEKPYKCPECGKSFSTSGNL | 1823 | X₁X₂X₃X₄X₅X₆X₇X₈X₉CX₁₀X₁₁CX₁₂X₁₃X₁₄X₁₅X₁₆TSGNLTEHX₁₇X₁₈X₁₉ | 2372 |
| acctgggca | CAC ACC | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QSGDLRRHX₁₇X₁₈ | |
| cat | TGG GCA | | QSGDLRRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDHLTTHX₁₇ | |
| | CAT | | GKSFSRSDHLTTHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DKKDLTRH | |
| | | | KCPECGKSFSDKKDLTRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆SKKALT | |
| | | | GEKPYKCPECGKSFSSKKALTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆RSDH | |
| | | | QRTHTGEKPYKCPECGKSFSRSD | | LTTHX₁₇X₁₈X₁₉HX₂₀X₂₁X₂₂X₂₃X₂₄X₈X₉CX₁₀X₁₁CX₁₂X₁₃X₁₄X₁₅X₁₆SK | |
| | | | HLTTHQRTHTGEKPYKCPECGKS | | KALTEHX₁₇X₁₈X₁₉HX₂₀X₂₁X₂₂X₂₃X₂₄X₂₅X₂₆X₂₇X₂₈X₂₉X₃₀ | |
| | | | FSSKKALTEHQRTHTGEKPTGKK | | | |
| | | | TS | | | |
| ctgccccca | CTG CCC | 1275 | LEPGEKPYKCPECGKSFSDCRDL | 1824 | X₁X₂X₃X₄X₅X₆X₇X₈X₉CX₁₀X₁₁CX₁₂X₁₃X₁₄X₁₅X₁₆DCRDLARHX₁₇X₁₈X₁₉ | 2373 |
| cctaccatt | CCA CCT | | ARHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆HKNALQNHX₁₇X₁₈ | |
| gcc | ACC ATT | | HKNALQNHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DKKDLTRHX₁₇ | |
| | GCC | | GKSFSDKKDLTRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TKNSLTEH | |
| | | | KCPECGKSFSTKNSLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSHSLT | |
| | | | GEKPYKCPECGKSFSTSHSLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆SKKH | |
| | | | QRTHTGEKPYKCPECGKSFSSKK | | LAEHX₁₇X₁₈X₁₉HX₂₀X₂₁X₂₂X₂₃X₂₄X₈X₉CX₁₀X₁₁CX₁₂X₁₃X₁₄X₁₅X₁₆RN | |
| | | | HLAEHQRTHTGEKPYKCPECGKS | | DALTEHX₁₇X₁₈X₁₉HX₂₀X₂₁X₂₂X₂₃X₂₄X₂₅X₂₆X₂₇X₂₈X₂₉X₃₀ | |
| | | | FSRNDALTEHQRTHTGEKPTGKK | | | |
| | | | TS | | | |
| tgggcacat | TGG GCA | 1276 | LEPGEKPYKCPECGKSFSTKNSL | 1825 | X₁X₂X₃X₄X₅X₆X₇X₈X₉CX₁₀X₁₁CX₁₂X₁₃X₁₄X₁₅X₁₆TKNSLTEHX₁₇X₁₈X₁₉ | 2374 |
| ctgccccca | CAT CTG | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSHSLTEHX₁₇X₁₈ | |
| cct | CCC CCA | | TSHSLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆SKKHLAEHX₁₇ | |
| | CCT | | GKSFSSKKHLAEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RNDALTEH | |
| | | | KCPECGKSFSRNDALTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSGNLT | |
| | | | GEKPYKCPECGKSFSTSGNLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆QSGD | |
| | | | QRTHTGEKPYKCPECGKSFSQSG | | LRRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | DLRRHQRTHTGEKPYKCPECGKS | | DHLTTHX₁₇X₁₈X₁₉HX₂₀X₂₁X₂₂X₂₃X₂₄X₂₅X₂₆X₂₇X₂₈X₂₉X₃₀ | |
| | | | FSRSDHLTTHQRTHTGEKPTGKK | | | |
| | | | TS | | | |
| acctgggca | ACC TGG | 1277 | LEPGEKPYKCPECGKSFSTSHSL | 1826 | X₁X₂X₃X₄X₅X₆X₇X₈X₉CX₁₀X₁₁CX₁₂X₁₃X₁₄X₁₅X₁₆TSHSLTEHX₁₇X₁₈X₁₉ | 2375 |
| catctgccc | GCA CAT | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆SKKHLAEHX₁₇X₁₈ | |
| cca | CTG CCC | | SKKHLAEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RNDALTEHX₁₇ | |
| | CCA | | GKSFSRNDALTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSGNLTEH | |
| | | | KCPECGKSFSTSGNLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QSGDLR | |
| | | | GEKPYKCPECGKSFSQSGDLRRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDH | |
| | | | QRTHTGEKPYKCPECGKSFSRSD | | LTTHX₁₇X₁₈X₁₉HX₂₀X₂₁X₂₂X₂₃X₂₄X₈X₉CX₁₀X₁₁CX₁₂X₁₃X₁₄X₁₅X₁₆DK | |
| | | | HLTTHQRTHTGEKPYKCPECGKS | | KDLTRHX₁₇X₁₈X₁₉HX₂₀X₂₁X₂₂X₂₃X₂₄X₂₅X₂₆X₂₇X₂₈X₂₉X₃₀ | |
| | | | FSDKKDLTRHQRTHTGEKPTGKK | | | |
| | | | TS | | | |
| gcacatctg | GCA CAT | 1278 | LEPGEKPYKCPECGKSFSDKKDL | 1827 | X₁X₂X₃X₄X₅X₆X₇X₈X₉CX₁₀X₁₁CX₁₂X₁₃X₁₄X₁₅X₁₆DKKDLTRHX₁₇X₁₈X₁₉ | 2376 |
| cccccacct | CTG CCC | | TRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TKNSLTEHX₁₇X₁₈ | |
| acc | CCA CCT | | TKNSLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSHSLTEHX₁₇ | |
| | ACC | | GKSFSTSHSLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆SKKHLAEH | |
| | | | KCPECGKSFSSKKHLAEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RNDALT | |
| | | | GEKPYKCPECGKSFSRNDALTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆TSGN | |
| | | | QRTHTGEKPYKCPECGKSFSTSG | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆QS | |
| | | | NLTEHQRTHTGEKPYKCPECGKS | | GDLRRHX₁₇X₁₈X₁₉HX₂₀X₂₁X₂₂X₂₃X₂₄X₂₅X₂₆X₂₇X₂₈X₂₉X₃₀ | |
| | | | FSQSGDLRRHQRTHTGEKPTGKK | | | |
| | | | TS | | | |
| cctaccatt | CCT ACC | 1279 | LEPGEKPYKCPECGKSFSQSGNL | 1828 | X₁X₂X₃X₄X₅X₆X₇X₈X₉CX₁₀X₁₁CX₁₂X₁₃X₁₄X₁₅X₁₆QSGNLTEHX₁₇X₁₈X₁₉ | 2377 |
| gcccatcgt | ATT GCC | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆SRRTCRAHX₁₇X₁₈ | |
| caa | CAT CGT | | SRRTCRAHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSGNLTEHX₁₇ | |
| | CAA | | GKSFSTSGNLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DCRDLARH | |
| | | | KCPECGKSFSDCRDLARHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆HKNALQ | |
| | | | GEKPYKCPECGKSFSHKNALQNH | | NHX₁₇X₁₈X₁₉HX₂₀X₂₁X₂₂X₂₃X₂₄X₈X₉CX₁₀X₁₁CX₁₂X₁₃X₁₄X₁₅X₁₆DKKD | |
| | | | QRTHTGEKPYKCPECGKSFSDKK | | LTRHX₁₇X₁₈X₁₉HX₂₀X₂₁X₂₂X₂₃X₂₄X₈X₉CX₁₀X₁₁CX₁₂X₁₃X₁₄X₁₅X₁₆TK | |
| | | | DLTRHQRTHTGEKPYKCPECGKS | | NSLTEHX₁₇X₁₈X₁₉HX₂₀X₂₁X₂₂X₂₃X₂₄X₂₅X₂₆X₂₇X₂₈X₂₉X₃₀ | |
| | | | FSTKNSLTEHQRTHTGEKPTGKK | | | |
| | | | TS | | | |
| catctgccc | CAT CTG | 1280 | LEPGEKPYKCPECGKSFSHKNAL | 1829 | X₁X₂X₃X₄X₅X₆X₇X₈X₉CX₁₀X₁₁CX₁₂X₁₃X₁₄X₁₅X₁₆HKNALQNHX₁₇X₁₈X₁₉ | 2378 |
| ccacctacc | CCC CCA | | QNHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆DKKDLTRHX₁₇X₁₈ | |
| att | CCT ACC | | DKKDLTRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TKNSLTEHX₁₇ | |
| | ATT | | GKSFSTKNSLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSHSLTEH | |
| | | | KCPECGKSFSTSHSLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆SKKHLA | |
| | | | GEKPYKCPECGKSFSSKKHLAEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆RNDA | |
| | | | QRTHTGEKPYKCPECGKSFSRND | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | ALTEHQRTHTGEKPYKCPECGKS | | GNLTEHX₁₇X₁₈X₁₉HX₂₀X₂₁X₂₂X₂₃X₂₄X₂₅X₂₆X₂₇X₂₈X₂₉X₃₀ | |
| | | | FSTSGNLTEHQRTHTGEKPTGKK | | | |
| | | | TS | | | |
| ccccactgg | CCC CAC | 1281 | LEPGEKPYKCPECGKSFSQSGDL | 1830 | X₁X₂X₃X₄X₅X₆X₇X₈X₉CX₁₀X₁₁CX₁₂X₁₃X₁₄X₁₅X₁₆QSGDLRRHX₁₇X₁₈X₁₉ | 2379 |
| cacacctgg | TGG CAC | | RRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDHLTTHX₁₇X₁₈ | |
| gca | ACC TGG | | RSDHLTTHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DKKDLTRHX₁₇ | |
| | GCA | | GKSFSDKKDLTRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆SKKALTEH | |
| | | | KCPECGKSFSSKKALTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDHLT | |
| | | | GEKPYKCPECGKSFSRSDHLTTH | | THX₁₇X₁₈X₁₉HX₂₀X₂₁X₂₂X₂₃X₂₄X₈X₉CX₁₀X₁₁CX₁₂X₁₃X₁₄X₁₅X₁₆SKKA | |
| | | | QRTHTGEKPYKCPECGKSFSSKK | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆SK | |
| | | | ALTEHQRTHTGEKPYKCPECGKS | | KHLAEHX₁₇X₁₈X₁₉HX₂₀X₂₁X₂₂X₂₃X₂₄X₂₅X₂₆X₂₇X₂₈X₂₉X₃₀ | |
| | | | FSSKKHLAEHQRTHTGEKPTGKK | | | |
| | | | TS | | | |
| cccccacct | CCC CCA | 1282 | LEPGEKPYKCPECGKSFSTSGNL | 1831 | X₁X₂X₃X₄X₅X₆X₇X₈X₉CX₁₀X₁₁CX₁₂X₁₃X₁₄X₁₅X₁₆TSGNLTEHX₁₇X₁₈X₁₉ | 2380 |
| accattgcc | CCT ACC | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆DCRDLARHX₁₇X₁₈ | |
| cat | ATT GCC | | DCRDLARHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆HKNALQNHX₁₇ | |
| | CAT | | GKSFSHKNALQNHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DKKDLTRH | |
| | | | KCPECGKSFSDKKDLTRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TKNSLT | |
| | | | GEKYKCPECGKSFSTKNSLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆TSHS | |
| | | | QRTHTGEKPYKCPECGKSFSTSH | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆SK | |
| | | | SLTEHQRTHTGEKPYKCPECGKS | | KHLAEHX₁₇X₁₈X₁₉HX₂₀X₂₁X₂₂X₂₃X₂₄X₂₅X₂₆X₂₇X₂₈X₂₉X₃₀ | |
| | | | FSSKKHLAEHQRTHTGEKPTGKK | | | |
| | | | TS | | | |
| cacacctgg | CAC ACC | 1283 | LEPGEKPYKCPECGKSFSSKKHL | 1832 | X₁X₂X₃X₄X₅X₆X₇X₈X₉CX₁₀X₁₁CX₁₂X₁₃X₁₄X₁₅X₁₆SKKHLAEHX₁₇X₁₈X₁₉ | 2381 |
| gcacatctg | TGG GCA | | AEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RNDALTEHX₁₇X₁₈ | |
| ccc | CAT CTG | | RNDALTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSGNLTEHX₁₇ | |
| | CCC | | GKSFSTSGNLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QSGDLRRH | |
| | | | KCPECGKSFSQSGDLRRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDHLT | |
| | | | GEKPYKCPECGKSFSRSDHLTTH | | THX₁₇X₁₈X₁₉HX₂₀X₂₁X₂₂X₂₃X₂₄X₈X₉CX₁₀X₁₁CX₁₂X₁₃X₁₄X₁₅X₁₆DKKD | |
| | | | QRTHTGEKPYKCPECGKSFSDKK | | LTRHX₁₇X₁₈X₁₉HX₂₀X₂₁X₂₂X₂₃X₂₄X₈X₉CX₁₀X₁₁CX₁₂X₁₃X₁₄X₁₅X₁₆SK | |
| | | | DLTRHQRTHTGEKPYKCPECGKS | | KALTEHX₁₇X₁₈X₁₉HX₂₀X₂₁X₂₂X₂₃X₂₄X₂₅X₂₆X₂₇X₂₈X₂₉X₃₀ | |
| | | | FSSKKALTEHQRTHTGEKPTGKK | | | |
| | | | TS | | | |
| attgcccat | ATT GCC | 1284 | LEPGEKPYKCPECGKSFSRNDAL | 1833 | X₁X₂X₃X₄X₅X₆X₇X₈X₉CX₁₀X₁₁CX₁₂X₁₃X₁₄X₁₅X₁₆RNDALTEHX₁₇X₁₈X₁₉ | 2382 |
| cgtcaacac | CAT CGT | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆SKKALTEHX₁₇X₁₈ | |
| ctg | CAA CAC | | SKKALTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QSGNLTEHX₁₇ | |
| | CTG | | GKSFSQSGNLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆SRRTCRAH | |
| | | | KCPECGKSFSSRRTCRAHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSGNLT | |
| | | | GEKPYKCPECGKSFSTSGNLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆DCRD | |
| | | | QRTHTGEKPYKCPECGKSFSDCR | | LARHX₁₇X₁₈X₁₉HX₂₀X₂₁X₂₂X₂₃X₂₄X₈X₉CX₁₀X₁₁CX₁₂X₁₃X₁₄X₁₅X₁₆HK | |
| | | | DLARHQRTHTGEKPYKCPECGKS | | NALQNHX₁₇X₁₈X₁₉HX₂₀X₂₁X₂₂X₂₃X₂₄X₂₅X₂₆X₂₇X₂₈X₂₉X₃₀ | |
| | | | FSHKNALQNHQRTHTGEKPTGKK | | | |
| | | | TS | | | |
| catcgtcaa | CAT CGT | 1285 | LEPGEKPYKCPECGKSFSHKNAL | 1834 | X₁X₂X₃X₄X₅X₆X₇X₈X₉CX₁₀X₁₁CX₁₂X₁₃X₁₄X₁₅X₁₆HKNALQNHX₁₇X₁₈X₁₉ | 2383 |
| cacctgcac | CAA CAC | | QNHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆SKKALTEHX₁₇X₁₈ | |
| att | CTG CAC | | SKKALTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RNDALTEHX₁₇ | |
| | ATT | | GKSFSRNDALTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆SKKALTEH | |
| | | | KCPECGKSFSSKKALTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QSGNLT | |
| | | | GEKPYKCPECGKSFSQSGNLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆SRRT | |
| | | | QRTHTGEKPYKCPECGKSFSSRR | | CRAHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | TCRAHQRTHTGEKPYKCPECGKS | | GNLTEHX₁₇X₁₈X₁₉HX₂₀X₂₁X₂₂X₂₃X₂₄X₂₅X₂₆X₂₇X₂₈X₂₉X₃₀ | |
| | | | FSTSGNLTEHQRTHTGEKPTGKK | | | |
| | | | TS | | | |
| accattgcc | ACC ATT | 1286 | LEPGEKPYKCPECGKSFSSKKAL | 1835 | X₁X₂X₃X₄X₅X₆X₇X₈X₉CX₁₀X₁₁CX₁₂X₁₃X₁₄X₁₅X₁₆SKKALTEHX₁₇X₁₈X₁₉ | 2384 |
| catcgtcaa | GCC CAT | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QSGNLTEHX₁₇X₁₈ | |
| cac | CGT CAA | | QSGNLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆SRRTCRAHX₁₇ | |
| | CAC | | GKSFSSRRTCRAHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSGNLTEH | |
| | | | KCPECGKSFSTSGNLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DCRDLA | |
| | | | GEKPYKCPECGKSFSDCRDLARH | | RHX₁₇X₁₈X₁₉HX₂₀X₂₁X₂₂X₂₃X₂₄X₈X₉CX₁₀X₁₁CX₁₂X₁₃X₁₄X₁₅X₁₆HKNA | |
| | | | QRTHTGEKPYKCPECGKSFSHKN | | LQNHX₁₇X₁₈X₁₉HX₂₀X₂₁X₂₂X₂₃X₂₄X₈X₉CX₁₀X₁₁CX₁₂X₁₃X₁₄X₁₅X₁₆DK | |
| | | | ALQNHQRTHTGEKPYKCPECGKS | | KDLTRHX₁₇X₁₈X₁₉HX₂₀X₂₁X₂₂X₂₃X₂₄X₂₅X₂₆X₂₇X₂₈X₂₉X₃₀ | |
| | | | FSDKKDLTRHQRTHTGEKPTGKK | | | |
| | | | TS | | | |
| gcccatcgt | GCC CAT | 1287 | LEPGEKPYKCPECGKSFSSKKAL | 1836 | X₁X₂X₃X₄X₅X₆X₇X₈X₉CX₁₀X₁₁CX₁₂X₁₃X₁₄X₁₅X₁₆SKKALTEHX₁₇X₁₈X₁₉ | 2385 |
| caacacctg | CGT CAA | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RNDALTEHX₁₇X₁₈ | |
| cac | CAC CTG | | RNDALTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆SKKALTEHX₁₇ | |
| | CAC | | GKSFSSKKALTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QSGNLTEH | |
| | | | KCPECGKSFSQSGNLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆SRRTCR | |
| | | | GEKPYKCPECGKSFSSRRTCRAH | | AHX₁₇X₁₈X₁₉HX₂₀X₂₁X₂₂X₂₃X₂₄X₈X₉CX₁₀X₁₁CX₁₂X₁₃X₁₄X₁₅X₁₆TSGN | |
| | | | QRTHTGEKPYKCPECGKSFSTSG | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆DC | |
| | | | NLTEHQRTHTGEKPYKCPECGKS | | RDLARHX₁₇X₁₈X₁₉HX₂₀X₂₁X₂₂X₂₃X₂₄X₂₅X₂₆X₂₇X₂₈X₂₉X₃₀ | |
| | | | FSDCRDLARHQRTHTGEKPTGKK | | | |
| | | | TS | | | |
| cagggtggt | CAG GGT | 1288 | LEPGEKPYKCPECGKSFSDPGAL | 1837 | X₁X₂X₃X₄X₅X₆X₇X₈X₉CX₁₀X₁₁CX₁₂X₁₃X₁₄X₁₅X₁₆DPGALVRHX₁₇X₁₈X₁₉ | 2386 |
| gtaggctgg | GGT GTA | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDHLTTHX₁₇X₁₈ | |
| gtc | GGC TGG | | RSDHLTTHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DPGHLVRHX₁₇ | |
| | GTC | | GKSFSDPGHLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QSSSLVRH | |
| | | | KCPECGKSFSQSSSLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSGHLV | |
| | | | GEKPYKCPECGKSFSTSGHLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆TSGH | |
| | | | QRTHTGEKPYKCPECGKSFSTSG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RA | |
| | | | HLVRHQRTHTGEKPYKCPECGKS | | DNLTEHX₁₇X₁₈X₁₉HX₂₀X₂₁X₂₂X₂₃X₂₄X₂₅X₂₆X₂₇X₂₈X₂₉X₃₀ | |
| | | | FSRADNLTEHQRTHTGEKPTGKK | | | |
| | | | TS | | | |
| ggtggtgta | GGT GGT | 1289 | LEPGEKPYKCPECGKSFSRADNL | 1838 | X₁X₂X₃X₄X₅X₆X₇X₈X₉CX₁₀X₁₁CX₁₂X₁₃X₁₄X₁₅X₁₆RADNLTEHX₁₇X₁₈X₁₉ | 2387 |
| ggctgggtc | GTA GGC | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆DPGALVRHX₁₇X₁₈ | |
| cag | TGG GTC | | DPGALVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDHLTTHX₁₇ | |
| | CAG | | GKSFSRSDHLTTHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DPGHLVRH | |
| | | | KCPECGKSFSDPGHLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QSSSLV | |
| | | | GEKPYKCPECGKSFSQSSSLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆TSGH | |
| | | | QRTHTGEKPYKCPECGKSFSTSG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | HLVRHQRTHTGEKPYKCPECGKS | | GHLVRHX₁₇X₁₈X₁₉HX₂₀X₂₁X₂₂X₂₃X₂₄X₂₅X₂₆X₂₇X₂₈X₂₉X₃₀ | |
| | | | FSTSGHLVRHQRTHTGEKPTGKK | | | |
| | | | TS | | | |
| aggctgggt | AGG CTG | 1290 | LEPGEKPYKCPECGKSFSSKKAL | 1839 | X₁X₂X₃X₄X₅X₆X₇X₈X₉CX₁₀X₁₁CX₁₂X₁₃X₁₄X₁₅X₁₆SKKALTEHX₁₇X₁₈X₁₉ | 2388 |
| ccagtgcag | GGT CCA | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RADNLTEHX₁₇X₁₈ | |
| cac | gtg CAG | | RADNLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDELVRHX₁₇ | |
| | CAC | | GKSFSRSDELVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSHSLTEH | |
| | | | KCPECGKSFSTSHSLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSGHLV | |
| | | | GEKPYKCPECGKSFSTSGHLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RNDA | |
| | | | QRTHTGEKPYKCPECGKSFSRND | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | ALTEHQRTHTGEKPYKCPECGKS | | DHLTNHX₁₇X₁₈X₁₉HX₂₀X₂₁X₂₂X₂₃X₂₄X₂₅X₂₆X₂₇X₂₈X₂₉X₃₀ | |
| | | | FSRSDHLTNHQRTHTGEKPTGKK | | | |
| | | | TS | | | |
| agcactgta | AGC ACT | 1291 | LEPGEKPYKCPECGKSFSDPGAL | 1840 | X₁X₂X₃X₄X₅X₆X₇X₈X₉CX₁₀X₁₁CX₁₂X₁₃X₁₄X₁₅X₁₆DPGALVRHX₁₇X₁₈X₁₉ | 2389 |
| gtcggccat | GTA GTC | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSGNLTEHX₁₇X₁₈ | |
| gtc | GGC CAT | | TSGNLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DPGHLVRHX₁₇ | |
| | GTC | | GKSFSDPGHLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DPGALVRH | |
| | | | KCPECGKSFSDPGALVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QSS SLV | |
| | | | GEKPYKCPECGKSFSQSSSLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆THLD | |
| | | | QRTHTGEKPYKCPECGKSFSTHL | | LIRHX₁₇X₁₈X₁₉HX₂₀X20X₂₂X₂₃X₂₄X₈X₉CX₁₀X₁₁CX₁₂X₁₃X₁₄X₁₅X₁₆ER | |
| | | | DLIRHQRTHTGEKPYKCPECGKS | | SHLREHX₁₇X₁₈X₁₉HX₂₀X20X₂₂X₂₃X₂₄X₂₅X₂₆X₂₇X₂₈X₂₉X₃₀ | |
| | | | FSERSHLREHQRTHTGEKPTGKK | | | |
| | | | TS | | | |
| actgtagtc | ACT GTA | 1292 | LEPGEKPYKCPECGKSFSTSGNL | 1841 | X₁X₂X₃X₄X₅X₆X₇X₈X₉CX₁₀X₁₁CX₁₂X₁₃X₁₄X₁₅X₁₆TSGNLTEHX₁₇X₁₈X₁₉ | 2390 |
| ggccatgtc | GTC GGC | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆DPGALVRHX₁₇X₁₈ | |
| cat | CAT GTC | | DPGALVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSGNLTEHX₁₇ | |
| | CAT | | GKSFSTSGNLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DPGHLVRH | |
| | | | KCPECGKSFSDPGHLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DPGALV | |
| | | | GEKPYKCPECGKSFSDPGALVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆QSSS | |
| | | | QRTHTGEKPYKCPECGKSFSQSS | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆TH | |
| | | | SLVRHQRTHTGEKPYKCPECGKS | | LDLIRHX₁₇X₁₈X₁₉HX₂₀X₂₁X₂₂X₂₃X₂₄X₂₅X₂₆X₂₇X₂₈X₂₉X₃₀ | |
| | | | FSTHLDLIRHQRTHTGEKPTGKK | | | |
| | | | TS | | | |
| gtagtcggc | GTA GTC | 1293 | LEPGEKPYKCPECGKSFSDPGAL | 1842 | X₁X₂X₃X₄X₅X₆X₇X₈X₉CX₁₀X₁₁CX₁₂X₁₃X₁₄X₁₅X₁₆DPGALVRHX₁₇X₁₈X₁₉ | 2391 |
| catgtccat | GGC CAT | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSGNLTEHX₁₇X₁₈ | |
| gtc | GTC CAT | | TSGNLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DPGALVRHX₁₇ | |
| | GTC | | GKSFSDPGALVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSGNLTEH | |
| | | | KCPECGKSFSTSGNLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DPGHLV | |
| | | | GEKPYKCPECGKSFSDPGHLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆DPGA | |
| | | | QRTHTGEKPYKCPECGKSFSDPG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆QS | |
| | | | ALVRHQRTHTGEKPYKCPECGKS | | SSLVRHX₁₇X₁₈X₁₉HX₂₀X₂₁X₂₂X₂₃X₂₄X₂₅X₂₆X₂₇X₂₈X₂₉X₃₀ | |
| | | | FSQSSSLVRHQRTHTGEKPTGKK | | | |
| | | | TS | | | |
| gtcggccat | GTC GGC | 1294 | LEPGEKPYKCPECGKSFSDPGNL | 1843 | X₁X₂X₃X₄X₅X₆X₇X₈X₉CX₁₀X₁₁CX₁₂X₁₃X₁₄X₁₅X₁₆DPGNLVRHX₁₇X₁₈X₁₉ | 2392 |
| gtccatgtc | CAT GTC | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆DPGALVRHX₁₇X₁₈ | |
| gac | CAT GTC | | DPGALVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSGNLTEHX₁₇ | |
| | GAC | | GKSFSTSGNLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DPGALVRH | |
| | | | KCPECGKSFSDPGALVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSGNLT | |
| | | | GEKPYKCPECGKSFSTSGNLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆DPGH | |
| | | | QRTHTGEKPYKCPECGKSFSDPG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆DP | |
| | | | HLVRHQRTHTGEKPYKCPECGKS | | GALVRHX₁₇X₁₈X₁₉HX₂₀X₂₁X₂₂X₂₃X₂₄X₂₅X₂₆X₂₇X₂₈X₂₉X₃₀ | |
| | | | FSDPGALVRHQRTHTGEKPTGKK | | | |
| | | | TS | | | |
| ggccatgtc | GGC CAT | 1295 | LEPGEKPYKCPECGKSFSRSDNL | 1844 | X₁X₂X₃X₄X₅X₆X₇X₈X₉CX₁₀X₁₁CX₁₂X₁₃X₁₄X₁₅X₁₆RSDNLVRHX₁₇X₁₈X₁₉ | 2393 |
| catgtcgac | GTC CAT | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆DPGNLVRHX₁₇X₁₈ | |
| gag | GTC GAC | | DPGNLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DPGALVRHX₁₇ | |
| | GAG | | GKSFSDPGALVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSGNLTEH | |
| | | | KCPECGKSFSTSGNLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DPGALV | |
| | | | GEKPYKCPECGKSFSDPGALVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆TSGN | |
| | | | QRTHTGEKPYKCPECGKSFSTSG | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆DP | |
| | | | NLTEHQRTHTGEKPYKCPECGKS | | GHLVRHX₁₇X₁₈X₁₉HX₂₀X₂₁X₂₂X₂₃X₂₄X₂₅X₂₆X₂₇X₂₈X₂₉X₃₀ | |
| | | | FSDPGHLVRHQRTHTGEKPTGKK | | | |
| | | | TS | | | |
| catgtccat | CAT GTC | 1296 | LEPGEKPYKCPECGKSFSTSGHL | 1845 | X₁X₂X₃X₄X₅X₆X₇X₈X₉CX₁₀X₁₁CX₁₂X₁₃X₁₄X₁₅X₁₆TSGHLVRHX₁₇X₁₈X₁₉ | 2394 |
| gtcgacgag | CAT GTC | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDNLVRHX₁₇X₁₈ | |
| ggt | GAC GAG | | RSDNLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DPGNLVRHX₁₇ | |
| | GGT | | GKSFSDPGNLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DPGALVRH | |
| | | | KCPECGKSFSDPGALVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSGNLT | |
| | | | GEKPYKCPECGKSFSTSGNLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆DPGA | |
| | | | QRTHTGEKPYKCPECGKSFSDPG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | ALVRHQRTHTGEKPYKCPECGKS | | GNLTEHX₁₇X₁₈X₁₉HX₂₀X₂₁X₂₂X₂₃X₂₄X₂₅X₂₆X₂₇X₂₈X₂₉X₃₀ | |
| | | | FSTSGNLTEHQRTHTGEKPTGKK | | | |
| | | | TS | | | |
| ctgcctcca | CTG CCT | 1297 | LEPGEKPYKCPECGKSFSSKKAL | 1846 | X₁X₂X₃X₄X₅X₆X₇X₈X₉CX₁₀X₁₁CX₁₂X₁₃X₁₄X₁₅X₁₆SKKALTEHX₁₇X₁₈X₁₉ | 2395 |
| cgccgcggc | CCA CGC | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆DPGHLVRHX₁₇X₁₈ | |
| cac | CGC GGC | | DPGHLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆HTGHLLEHX₁₇ | |
| | CAC | | GKSFSHTGHLLEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆HTGHLLEH | |
| | | | KCPECGKSFSHTGHLLEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSHSLT | |
| | | | GEKPYKCPECGKSFSTSHSLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆TKNS | |
| | | | QRTHTGEKPYKCPECGKSFSTKN | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆RN | |
| | | | SLTEHQRTHTGEKPYKCPECGKS | | DALTEHX₁₇X₁₈X₁₉HX₂₀X₂₁X₂₂X₂₃X₂₄X₂₅X₂₆X₂₇X₂₈X₂₉X₃₀ | |
| | | | FSRNDALTEHQRTHTGEKPTGKK | | | |
| | | | TS | | | |
| cctccacgc | CCT CCA | 1298 | LEPGEKPYKCPECGKSFSRNDAL | 1847 | X₁X₂X₃X₄X₅X₆X₇X₈X₉CX₁₀X₁₁CX₁₂X₁₃X₁₄X₁₅X₁₆RNDALTEHX₁₇X₁₈X₁₉ | 2396 |
| cgcggccac | CGC CGC | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆SKKALTEHX₁₇X₁₈ | |
| ctg | GGC CAC | | SKKALTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DPGHLVRHX₁₇ | |
| | CTG | | GKSFSDPGHLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆HTGHLLEH | |
| | | | KCPECGKSFSHTGHLLEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆HTGHLL | |
| | | | GEKPYKCPECGKSFSHTGHLLEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆TSHS | |
| | | | QRTHTGEKPYKCPECGKSFSTSH | | LTEHX₁₇X₁₈X₁₉HX₂₀X₂₁X₂₂X₂₃X₂₄X₈X₉CX₁₀X₁₁CX₁₂XEX₁₄X₁₅X₁₆TK | |
| | | | SLTEHQRTHTGEKPYKCPECGKS | | N5LTEHX₁₇X₁₈X₁₉HX₂₀X₂₁X₂₂X₂₃X₂₄X₂₅X₂₆X₂₇X₂₈X₂₉X₃₀ | |
| | | | FSTKNSLTEHQRTHTGEKPTGKK | | | |
| | | | TS | | | |
| ccacgccgc | CCA CGC | 1299 | LEPGEKPYKCPECGKSFSSKKHL | 1848 | X₁X₂X₃X₄X₅X₆X₇X₈X₉CX₁₀X₁₁CX₁₂X₁₃X₁₄X₁₅X₁₆SKKHLAEHX₁₇X₁₈X₁₉ | 2397 |
| ggccacctg | CGC GGC | | AEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RNDALTEHX₁₇X₁₈ | |
| ccc | CAC CTG | | RNDALTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆SKKALTEHX₁₇ | |
| | CCC | | GKSFSSKKALTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DPGHLVRH | |
| | | | KCPECGKSFSDPGHLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆HTGHLL | |
| | | | GEKPYKCPECGKSFSHTGHLLEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆HTGH | |
| | | | QRTHTGEKPYKCPECGKSFSHTG | | LLEHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | HLLEHQRTHTGEKPYKCPECGKS | | HSLTEHX₁₇X₁₈X₁₉HX₂₀X₂₁X₂₂X₂₃X₂₄X₂₅X₂₆X₂₇X₂₈X₂₉X₃₀ | |
| | | | FSTSHSLTEHQRTHTGEKPTGKK | | | |
| | | | TS | | | |
| cggccacct | CGG CCA | 1300 | LEPGEKPYKCPECGKSFSRSDHL | 1849 | X₁X₂X₃X₄X₅X₆X₇X₈X₉CX₁₀X₁₁CX₁₂X₁₃X₁₄X₁₅X₁₆RSDHLTTHX₁₇X₁₈X₁₉ | 2398 |
| gccctaccc | CCT GCC | | TTHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆SKKHLAEHX₁₇X₁₈ | |
| tgg | CTA CCC | | SKKHLAEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QNSTLTEHX₁₇ | |
| | TGG | | GKSFSQNSTLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DCRDLARH | |
| | | | KCPECGKSFSDCRDLARHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TKNSLT | |
| | | | GEKPYKCPECGKSFSTKNSLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆TSHS | |
| | | | QRTHTGEKPYKCPECGKSFSTSH | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | SLTEHQRTHTGEKPYKCPECGKS | | DKLTEHX₁₇X₁₈X₁₉HX₂₀X₂₁X₂₂X₂₃X₂₄X₂₅X₂₆X₂₇X₂₈X₂₉X₃₀ | |
| | | | FSRSDKLTEHQRTHTGEKPTGKK | | | |
| | | | TS | | | |
| acgccgcgg | ACG CCG | 1301 | LEPGEKPYKCPECGKSFSQNSTL | 1850 | X₁X₂X₃X₄X₅X₆X₇X₈X₉CX₁₀X₁₁CX₁₂X₁₃X₁₄X₁₅X₁₆QNSTLTEHX₁₇X₁₈X₁₉ | 2399 |
| ccacctgcc | CGG CCA | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆DCRDLARHX₁₇X₁₈ | |
| cta | CCT GCC | | DCRDLARHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TKNSLTEHX₁₇ | |
| | CTA | | GKSFSTKNSLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSHSLTEH | |
| | | | KCPECGKSFSTSHSLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDKLT | |
| | | | GEKPYKCPECGKSFSRSDKLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆RNDT | |
| | | | QRTHTGEKPYKCPECGKSFSRND | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆RT | |
| | | | TLTEHQRTHTGEKPYKCPECGKS | | DTLRDHX₁₇X₁₃X₁₉HX₂₀X₂₁X₂₂X₂₃X₂₄X₂₅X₂₆X₂₇X₂₈X₂₉X₃₀ | |
| | | | FSRTDTLRDHQRTHTGEKPTGKK | | | |
| | | | TS | | | |
| ccgcggcca | CCG CGG | 1302 | LEPGEKPYKCPECGKSFSSKKHL | 1851 | X₁X₂X₃X₄X₅X₆X₇X₈X₉CX₁₀X₁₁CX₁₂X₁₃X₁₄X₁₅X₁₆SKKHLAEHX₁₇X₁₈X₁₉ | 2400 |
| cctgcccta | CCA CCT | | AEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QNSTLTEHX₁₇X₁₈ | |
| ccc | GCC CTA | | QNSTLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DCRDLARHX₁₇ | |
| | CCC | | GKSFSDCRDLARHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TKNSLTEH | |
| | | | KCPECGKSFSTKNSLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSHSLT | |
| | | | GEKPYKCPECGKSFSTSHSLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆RSDK | |
| | | | QRTHTGEKPYKCPECGKSFSRSD | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆RN | |
| | | | KLTEHQRTHTGEKPYKCPECGKS | | DTLTEHX₁₇X₁₈X₁₉HX₂₀X₂₁X₂₂X₂₃X₂₄X₂₅X₂₆X₂₇X₂₈X₂₉X₃₀ | |
| | | | FSRNDTLTEHQRTHTGEKPTGKK | | | |
| | | | TS | | | |
| ccacctgcc | CCA CCT | 1303 | LEPGEKPYKCPECGKSFSRSDDL | 1852 | X₁X₂X₃X₄X₅X₆X₇X₈X₉CX₁₀X₁₁CX₁₂X₁₃X₁₄X₁₅X₁₆RSDDLVRHX₁₇X₁₈X₁₉ | 2401 |
| ctaccctgg | GCC CTA | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDHLTTHX₁₇X₁₈ | |
| gcg | CCC TGG | | RSDHLTTHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆SKKHLAEHX₁₇ | |
| | GCG | | GKSFSSKKHLAEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QNSTLTEH | |
| | | | KCPECGKSFSQNSTLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DCRDLA | |
| | | | GEKPYKCPECGKSFSDCRDLARH | | RHX₁₇X₁₈X₁₉HX₂₀X₂₁X₂₂X₂₃X₂₄X₈X₉CX₁₀X₁₁CX₁₂X₁₃X₁₄X₁₅X₁₆TKNS | |
| | | | QRTHTGEKPYKCPECGKSFSTKN | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | SLTEHQRTHTGEKPYKCPECGKS | | HSLTEHX₁₇X₁₈X₁₉HX₂₀X₂₁X₂₂X₂₃X₂₄X₂₅X₂₆X₂₇X₂₈X₂₉X₃₀ | |
| | | | FSTSHSLTEHQRTHTGEKPTGKK | | | |
| | | | TS | | | |
| cctgcccta | CCT GCC | 1304 | LEPGEKPYKCPECGKSFSSKKHL | 1853 | X₁X₂X₃X₄X₅X₆X₇X₈X₉CX₁₀X₁₁CX₁₂X₁₃X₁₄X₁₅X₁₆SKKHLAEHX₁₇X₁₈X₁₉ | 2402 |
| ccctgggcg | CTA CCC | | AEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDDLVRHX₁₇X₁₈ | |
| ccc | TGG gcg | | RSDDLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDHLTTHX₁₇ | |
| | CCC | | GKSFSRSDHLTTHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆SKKHLAEH | |
| | | | KCPECGKSFSSKKHLAEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QNSTLT | |
| | | | GEKPYKCPECGKSFSQNSTLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆DCRD | |
| | | | QRTHTGEKPYKCPECGKSFSDCR | | LARHX₁₇X₁₈X₁₉HX₂₀X₂₁X₂₂X₂₃X₂₄X₈X₉CX₁₀X₁₁CX₁₂X₁₃X₁₄X₁₅X₁₆TK | |
| | | | DLARHQRTHTGEKPYKCPECGKS | | NSLTEHX₁₇X₁₃X₁₉HX₂₀X₂₁X₂₂X₂₃X₂₄X₂₅X₂₆X₂₇X₂₈X₂₉X₃₀ | |
| | | | FSTKNSLTEHQRTHTGEKPTGKK | | | |
| | | | TS | | | |
| gccctaccc | GCC CTA | 1305 | LEPGEKPYKCPECGKSFSDKKDL | 1854 | X₁X₂X₃X₄X₅X₆X₇X₈X₉CX₁₀X₁₁CX₁₂X₁₃X₁₄X₁₅X₁₆DKKDLTRHX₁₇X₁₈X₁₉ | 2403 |
| tgggcgccc | CCC TGG | | TRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆SKKHLAEHX₁₇X₁₈ | |
| acc | gcg CCC | | SKKHLAEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDDLVRHX₁₇ | |
| | ACC | | GKSFSRSDDLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDHLTTH | |
| | | | KCPECGKSFSRSDHLTTHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆SKKHLA | |
| | | | GEKPYKCPECGKSFSSKKHLAEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆QNST | |
| | | | QRTHTGEKPYKCPECGKSFSQNS | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆DC | |
| | | | TLTEHQRTHTGEKPYKCPECGKS | | RDLARHX₁₇X₁₃X₁₉HX₂₀X₂₁X₂₂X₂₃X₂₄X₂₅X₂₆X₂₇X₂₈X₂₉X₃₀ | |
| | | | FSDCRDLARHQRTHTGEKPTGKK | | | |
| | | | TS | | | |
| ccctgggcg | CCC TGG | 1306 | LEPGEKPYKCPECGKSFSRKDNL | 1855 | X₁X₂X₃X₄X₅X₆X₇X₈X₉CX₁₀X₁₁CX₁₂X₁₃X₁₄X₁₅X₁₆RKDNLKNHX₁₇X₁₈X₁₉ | 2404 |
| cccaccccg | gcg CCC | | KNHQRTHTGEKPYKCPECGKSFS | | HX₂₀X20X₂₂X₂₃X₂₄X₈X₉CX₁₀X₁₁CX₁₂X₁₃X₁₄X₁₅X₁₆RNDTLTEHX₁₇X₁₈ | |
| aag | ACC CCG | | RNDTLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DKKDLTRHX₁₇ | |
| | AAG | | GKSFSDKKDLTRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆SKKHLAEH | |
| | | | KCPECGKSFSSKKHLAEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDDLV | |
| | | | GEKPYKCPECGKSFSRSDDLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDH | |
| | | | QRTHTGEKPYKCPECGKSFSRSD | | LTTHX₁₇X₁₈X₁₉HX₂₀X₂₁X₂₂X₂₃X₂₄X₈X₉CX₁₀X₁₁CX₁₂XEX₁₄X₁₅X₁₆SK | |
| | | | HLTTHQRTHTGEKPYKCPECGKS | | KHLAEHX₁₇X₁₈X₁₉HX₂₀X₂₁X₂₂X₂₃X₂₄X₂₅X₂₆X₂₇X₂₈X₂₉X₃₀ | |
| | | | FSSKKHLAEHQRTHTGEKPTGKK | | | |
| | | | TS | | | |
| tgggcgccc | TGG gcg | 1307 | LEPGEKPYKCPECGKSFSDCRDL | 1856 | X₁X₂X₃X₄X₅X₆X₇X₈X₉CX₁₀X₁₁CX₁₂X₁₃X₁₄X₁₅X₁₆DCRDLARHX₁₇X₁₈X₁₉ | 2405 |
| accccgaag | CCC ACC | | ARHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RKDNLKNHX₁₇X₁₈ | |
| gcc | CCG AAG | | RKDNLKNHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RNDTLTEHX₁₇ | |
| | GCC | | GKSFSRNDTLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DKKDLTRH | |
| | | | KCPECGKSFSDKKDLTRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆SKKHLA | |
| | | | GEKPYKCPECGKSFSSKKHLAEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆RSDD | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | DLVRHQRTHTGEKPYKCPECGKS | | DHLTTHX₁₇X₁₈X₁₉HX₂₀X₂₁X₂₂X₂₃X₂₄X₂₅X₂₆X₂₇X₂₈X₂₉X₃₀ | |
| | | | FSRSDHLTTHQRTHTGEKPTGKK | | | |
| | | | TS | | | |
| ctaccctgg | CTA CCC | 1308 | LEPGEKPYKCPECGKSFSRNDTL | 1857 | X₁X₂X₃X₄X₅X₆X₇X₈X₉CX₁₀X₁₁CX₁₂X₁₃X₁₄X₁₅X₁₆RNDTLTEHX₁₇X₁₈X₁₉ | 2406 |
| gcgcccacc | TGG gcg | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆DKKDLTRHX₁₇X₁₈ | |
| ccg | CCC ACC | | DKKDLTRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆SKKHLAEHX₁₇ | |
| | CCG | | GKSFSSKKHLAEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDDLVRH | |
| | | | KCPECGKSFSRSDDLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDHLT | |
| | | | GEKPYKCPECGKSFSRSDHLTTH | | THX₁₇X₁₈X₁₉HX₂₀X₂₁X₂₂X₂₃X₂₄X₈X₉CX₁₀X₁₁CX₁₂X₁₃X₁₄X₁₅X₁₆SKKH | |
| | | | QRTHTGEKPYKCPECGKSFSSKK | | LAEHX₁₇X₁₈X₁₉HX₂₀X₂₁X₂₂X₂₃X₂₄X₈X₉CX₁₀X₁₁CX₁₂X₁₃X₁₄X₁₅X₁₆QN | |
| | | | HLAEHQRTHTGEKPYKCPECGKS | | STLTEHX₁₇X₁₃X₁₉HX₂₀X₂₁X₂₂X₂₃X₂₄X₂₅X₂₆X₂₇X₂₈X₂₉X₃₀ | |
| | | | FSQNSTLTEHQRTHTGEKPTGKK | | | |
| | | | TS | | | |
| cccaccccg | CCC ACC | 1309 | LEPGEKPYKCPECGKSFSDCRDL | 1858 | X₁X₂X₃X₄X₅X₆X₇X₈X₉CX₁₀X₁₁CX₁₂X₁₃X₁₄X₁₅X₁₆DCRDLARHX₁₇X₁₈X₁₉ | 2407 |
| aaggccccc | CCG AAG | | ARHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆SKKHLAEHX₁₇X₁₈ | |
| gcc | GCC CCC | | SKKHLAEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DCRDLARHX₁₇ | |
| | GCC | | GKSFSDCRDLARHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RKDNLKNH | |
| | | | KCPECGKSFSRKDNLKNHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RNDTLT | |
| | | | GEKPYKCPECGKSFSRNDTLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆DKKD | |
| | | | QRTHTGEKPYKCPECGKSFSDKK | | LTRHX₁₇X₁₈X₁₉HX₂₀X₂₁X₂₂X₂₃X₂₄X₈X₉CX₁₀X₁₁CX₁₂X₁₃X₁₄X₁₅X₁₆SK | |
| | | | DLTRHQRTHTGEKPYKCPECGKS | | KHLAEHX₁₇X₁₈X₁₉HX₂₀X₂₁X₂₂X₂₃X₂₄X₂₅X₂₆X₂₇X₂₈X₂₉X₃₀ | |
| | | | FSSKKHLAEHQRTHTGEKPTGKK | | | |
| | | | TS | | | |
| gcgcccacc | gcg CCC | 1310 | LEPGEKPYKCPECGKSFSSKKHL | 1859 | X₁X₂X₃X₄X₅X₆X₇X₈X₉CX₁₀X₁₁CX₁₂X₁₃X₁₄X₁₅X₁₆SKKHLAEHX₁₇X₁₈X₁₉ | 2408 |
| ccgaaggcc | ACC CCG | | AEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆DCRDLARHX₁₇X₁₈ | |
| ccc | AAG GCC | | DCRDLARHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RKDNLKNHX₁₇ | |
| | CCC | | GKSFSRKDNLKNHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RNDTLTEH | |
| | | | KCPECGKSFSRNDTLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DKKDLT | |
| | | | GEKPYKCPECGKSFSDKKDLTRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆SKKH | |
| | | | QRTHTGEKPYKCPECGKSFSSKK | | LAEHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | HLAEHQRTHTGEKPYKCPECGKS | | DDLVRHX₁₇X₁₈X₁₉HX₂₀X₂₁X₂₂X₂₃X₂₄X₂₅X₂₆X₂₇X₂₈X₂₉X₃₀ | |
| | | | FSRSDDLVRHQRTHTGEKPTGKK | | | |
| | | | TS | | | |
| cctaccctg | CCT ACC | 1311 | LEPGEKPYKCPECGKSFSSKKHL | 1860 | X₁X₂X₃X₄X₅X₆X₇X₈X₉CX₁₀X₁₁CX₁₂X₁₃X₁₄X₁₅X₁₆SKKHLAEHX₁₇X₁₈X₁₉ | 2409 |
| ggcgcccac | CTG GGC | | AEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆SKKALTEHX₁₇X₁₈ | |
| ccc | GCC CAC | | SKKALTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DCRDLARHX₁₇ | |
| | CCC | | GKSFSDCRDLARHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DPGHLVRH | |
| | | | KCPECGKSFSDPGHLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RNDALT | |
| | | | GEKPYKCPECGKSFSRNDALTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆DKKD | |
| | | | QRTHTGEKPYKCPECGKSFSDKK | | LTRHX₁₇X₁₈X₁₉HX₂₀X₂₁X₂₂X₂₃X₂₄X₈X₉CX₁₀X₁₁CX₁₂X₁₃X₁₄X₁₅X₁₆TK | |
| | | | DLTRHQRTHTGEKPYKCPECGKS | | NSLTEHX₁₇X₁₈X₁₉HX₂₀X₂₁X₂₂X₂₃X₂₄X₂₅X₂₆X₂₇X₂₈X₂₉X₃₀ | |
| | | | FSTKNSLTEHQRTHTGEKPTGKK | | | |
| | | | TS | | | |
| ctgggcgcc | CTG GGC | 1312 | LEPGEKPYKCPECGKSFSDPGHL | 1861 | X₁X₂X₃X₄X₅X₆X₇X₈X₉CX₁₀X₁₁CX₁₂X₁₃X₁₄X₁₅X₁₆DPGHLVRHX₁₇X₁₈X₁₉ | 2410 |
| caccccgaa | GCC CAC | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QSSNLVRHX₁₇X₁₈ | |
| ggc | CCC GAA | | QSSNLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆SKKHLAEHX₁₇ | |
| | GGC | | GKSFSSKKHLAEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆SKKALTEH | |
| | | | KCPECGKSFSSKKALTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DCRDLA | |
| | | | GEKPYKCPECGKSFSDCRDLARH | | RHX₁₇X₁₈X₁₉HX₂₀X₂₁X₂₂X₂₃X₂₄X₈X₉CX₁₀X₁₁CX₁₂X₁₃X₁₄X₁₅X₁₆DPGH | |
| | | | QRTHTGEKPYKCPECGKSFSDPG | | LVRHX₁₇X₁₈X₁₉HX₂₀X₂₁X₂₂X₂₃X₂₄X₈X₉CX₁₀X₁₁CX₁₂XEX₁₄X₁₅X₁₆RN | |
| | | | HLVRHQRTHTGEKPYKCPECGKS | | DALTEHX₁₇X₁₈X₁₉HX₂₀X₂₁X₂₂X₂₃X₂₄X₂₅X₂₆X₂₇X₂₈X₂₉X₃₀ | |
| | | | FSRNDALTEHQRTHTGEKPTGKK | | | |
| | | | TS | | | |
| accctgggc | ACC CTG | 106 | LEPGEKPYKCPECGKSFSQSSNL | 107 | X₁X₂X₃X₄X₅X₆X₇X₈X₉CX₁₀X₁₁CX₁₂X₁₃X₁₄X₁₅X₁₆QSSNLVRHX₁₇X₁₈X₁₉ | 314 |
| gcccacccc | GGC GCC | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆SKKHLAEHX₁₇X₁₈ | |
| gaa | CAC CCC | | SKKHLAEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆SKKALTEHX₁₇ | |
| | GAA | | GKSFSSKKALTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DCRDLARH | |
| | | | KCPECGKSFSDCRDLARHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DPGHLV | |
| | | | GEKPYKCPECGKSFSDPGHLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RNDA | |
| | | | QRTHTGEKPYKCPECGKSFSRND | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆DK | |
| | | | ALTEHQRTHTGEKPYKCPECGKS | | KDLTRHX₁₇X₁₈X₁₉HX₂₀X₂₁X₂₂X₂₃X₂₄X₂₅X₂₆X₂₇X₂₈X₂₉X₃₀ | |
| | | | FSDKKDLTRHQRTHTGEKPTGKK | | | |
| | | | TS | | | |
| ggcgcccac | GGC GCC | 1313 | LEPGEKPYKCPECGKSFSSKKHL | 1862 | X₁X₂X₃X₄X₅X₆X₇X₈X₉CX₁₀X₁₁CX₁₂X₁₃X₁₄X₁₅X₁₆SKKHLAEHX₁₇X₁₈X₁₉ | 2411 |
| cccgaaggc | CAC CCC | | AEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆DPGHLVRHX₁₇X₁₈ | |
| ccc | GAA GGC | | DPGHLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QSSNLVRHX₁₇ | |
| | CCC | | GKSFSQSSNLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆SKKHLAEH | |
| | | | KCPECGKSFSSKKHLAEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆SKKALT | |
| | | | GEKPYKCPECGKSFSSKKALTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆DCRD | |
| | | | QRTHTGEKPYKCPECGKSFSDCR | | LARHX₁₇X₁₈X₁₉HX₂₀X₂₁X₂₂X₂₃X₂₄X₈X₉CX₁₀X₁₁CX₁₂X₁₃X₁₄X₁₅X₁₆DP | |
| | | | DLARHQRTHTGEKPYKCPECGKS | | GHLVRHX₁₇X₁₈X₁₉HX₂₀X₂₁X₂₂X₂₃X₂₄X₂₅X₂₆X₂₇X₂₈X₂₉X₃₀ | |
| | | | FSDPGHLVRHQRTHTGEKPTGKK | | | |
| | | | TS | | | |
| caccccgaa | CAC CCC | 1314 | LEPGEKPYKCPECGKSFSTKNSL | 1863 | X₁X₂X₃X₄X₅X₆X₇X₈X₉CX₁₀X₁₁CX₁₂X₁₃X₁₄X₁₅X₁₆TKNSLTEHX₁₇X₁₈X₁₉ | 2412 |
| ggcccccgc | GAA GGC | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆HTGHLLEHX₁₇X₁₈ | |
| cct | CCC CGC | | HTGHLLEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆SKKHLAEHX₁₇ | |
| | CCT | | GKSFSSKKHLAEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DPGHLVRH | |
| | | | KCPECGKSFSDPGHLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QSSNLV | |
| | | | GEKPYKCPECGKSFSQSSNLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆SKKH | |
| | | | QRTHTGEKPYKCPECGKSFSSKK | | LAEHX₁₇X₁₈X₁₉HX₂₀X₂₁X₂₂X₂₃X₂₄X₈X₉CX₁₀X₁₁CX₁₂X₁₃X₁₄X₁₅X₁₆SK | |
| | | | HLAEHQRTHTGEKPYKCPECGKS | | KALTEHX₁₇X₁₈X₁₉HX₂₀X₂₁X₂₂X₂₃X₂₄X₂₅X₂₆X₂₇X₂₈X₂₉X₃₀ | |
| | | | FSSKKALTEHQRTHTGEKPTGKK | | | |
| | | | TS | | | |
| gcccacccc | GCC CAC | 1315 | LEPGEKPYKCPECGKSFSHTGHL | 1864 | X₁X₂X₃X₄X₅X₆X₇X₈X₉CX₁₀X₁₁CX₁₂X₁₃X₁₄X₁₅X₁₆HTGHLLEHX₁₇X₁₈X₁₉ | 2413 |
| gaaggcccc | CCC GAA | | LEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆SKKHLAEHX₁₇X₁₈ | |
| cgc | GGC CCC | | SKKHLAEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DPGHLVRHX₁₇ | |
| | CGC | | GKSFSDPGHLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QSSNLVRH | |
| | | | KCPECGKSFSQSSNLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆SKKHLA | |
| | | | GEKPYKCPECGKSFSSKKHLAEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆SKKA | |
| | | | QRTHTGEKPYKCPECGKSFSSKK | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆DC | |
| | | | ALTEHQRTHTGEKPYKCPECGKS | | RDLARHX₁₇X₁₈X₁₉HX₂₀X₂₁X₂₂X₂₃X₂₄X₂₅X₂₆X₂₇X₂₈X₂₉X₃₀ | |
| | | | FSDCRDLARHQRTHTGEKPTGKK | | | |
| | | | TS | | | |
| cccgaaggc | CCC GAA | 1316 | LEPGEKPYKCPECGKSFSRNDTL | 1865 | X₁X₂X₃X₄X₅X₆X₇X₈X₉CX₁₀X₁₁CX₁₂X₁₃X₁₄X₁₅X₁₆RNDTLTEHX₁₇X₁₈X₁₉ | 2414 |
| ccccgccct | GGC CCC | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TKNSLTEHX₁₇X₁₈ | |
| ccg | CGC CCT | | TKNSLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆HTGHLLEHX₁₇ | |
| | CCG | | GKSFSHTGHLLEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆SKKHLAEH | |
| | | | KCPECGKSFSSKKHLAEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DPGHLV | |
| | | | GEKPYKCPECGKSFSDPGHLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆QSSN | |
| | | | QRTHTGEKPYKCPECGKSFSQSS | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆SK | |
| | | | NLVRHQRTHTGEKPYKCPECGKS | | KHLAEHX₁₇X₁₈X₁₉HX₂₀X₂₁X₂₂X₂₃X₂₄X₂₅X₂₆X₂₇X₂₈X₂₉X₃₀ | |
| | | | FSSKKHLAEHQRTHTGEKPTGKK | | | |
| | | | TS | | | |
| gaaggcccc | GAA GGC | 1317 | LEPGEKPYKCPECGKSFSSKKHL | 1866 | X₁X₂X₃X₄X₅X₆X₇X₈X₉CX₁₀X₁₁CX₁₂X₁₃X₁₄X₁₅X₁₆SKKHLAEHX₁₇X₁₈X₁₉ | 2415 |
| cgccctccg | CCC CGC | | AEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RNDTLTEHX₁₇X₁₈ | |
| ccc | CCT CCG | | RNDTLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TKNSLTEHX₁₇ | |
| | CCC | | GKSFSTKNSLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆HTGHLLEH | |
| | | | KCPECGKSFSHTGHLLEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆SKKHLA | |
| | | | GEKPYKCPECGKSFSSKKHLAEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆DPGH | |
| | | | QRTHTGEKPYKCPECGKSFSDPG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆QS | |
| | | | HLVRHQRTHTGEKPYKCPECGKS | | SNLVRHX₁₇X₁₈X₁₉HX₂₀X₂₁X₂₂X₂₃X₂₄X₂₅X₂₆X₂₇X₂₈X₂₉X₃₀ | |
| | | | FSQSSNLVRHQRTHTGEKPTGKK | | | |
| | | | TS | | | |
| cctgggcgc | CCT ggg | 1318 | LEPGEKPYKCPECGKSFSRSDHL | 1867 | X₁X₂X₃X₄X₅X₆X₇X₈X₉CX₁₀X₁₁CX₁₂X₁₃X₁₄X₁₅X₁₆RSDHLTNHX₁₁X1BX₁₉ | 2416 |
| ccaccccga | CGC CCA | | TNHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QSGHLTEHX₁₇X₁₈ | |
| agg | CCC CGA | | QSGHLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆SKKHLAEHX₁₇ | |
| | AGG | | GKSFSSKKHLAEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSHSLTEH | |
| | | | KCPECGKSFSTSHSLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆HTGHLL | |
| | | | GEKPYKCPECGKSFSHTGHLLEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆RSDK | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆TK | |
| | | | KLVRHQRTHTGEKPYKCPECGKS | | NSLTEHX₁₇X₁₈X₁₉HX₂₀X₂₁X₂₂X₂₃X₂₄X₂₅X₂₆X₂₇X₂₈X₂₉X₃₀ | |
| | | | FSTKNSLTEHQRTHTGEKPTGKK | | | |
| | | | TS | | | |
| gggcgccca | ggg CGC | 1319 | LEPGEKPYKCPECGKSFSSKKHL | 1868 | X₁X₂X₃X₄X₅X₆X₇X₈X₉CX₁₀X₁₁CX₁₂X₁₃X₁₄X₁₅X₁₆SKKHLAEHX₁₇X₁₈X₁₉ | 2417 |
| ccccgaagg | CCA CCC | | AEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDHLTNHX₁₇X₁₈ | |
| ccc | CGA AGG | | RSDHLTNHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QSGHLTEHX₁₇ | |
| | CCC | | GKSFSQSGHLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆SKKHLAEH | |
| | | | KCPECGKSFSSKKHLAEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSHSLT | |
| | | | GEKPYKCPECGKSFSTSHSLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆HTGH | |
| | | | QRTHTGEKPYKCPECGKSFSHTG | | LLEHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | HLLEHQRTHTGEKPYKCPECGKS | | DKLVRHX₁₇X₁₈X₁₉HX₂₀X₂₁X₂₂X₂₃X₂₄X₂₅X₂₆X₂₇X₂₈X₂₉X₃₀ | |
| | | | FSRSDKLVRHQRTHTGEKPTGKK | | | |
| | | | TS | | | |
| cgcccaccc | CGC CCA | 1320 | LEPGEKPYKCPECGKSFSRNDTL | 1869 | X₁X₂X₃X₄X₅X₆X₇X₈X₉CX₁₀X₁₁CX₁₂X₁₃X₁₄X₁₅X₁₆RNDTLTEHX₁₇X₁₈X₁₉ | 2418 |
| cgaaggccc | CCC CGA | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆SKKHLAEHX₁₇X₁₈ | |
| ccg | AGG CCC | | SKKHLAEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDHLTNHX₁₇ | |
| | CCG | | GKSFSRSDHLTNHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QSGHLTEH | |
| | | | KCPECGKSFSQSGHLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆SKKHLA | |
| | | | GEKPYKCPECGKSFSSKKHLAEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆TSHS | |
| | | | QRTHTGEKPYKCPECGKSFSTSH | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆HT | |
| | | | SLTEHQRTHTGEKPYKCPECGKS | | GHLLEHX₁₇X₁₃X₁₉HX₂₀X₂₁X₂₂X₂₃X₂₄X₂₅X₂₆X₂₇X₂₈X₂₉X₃₀ | |
| | | | FSHTGHLLEHQRTHTGEKPTGKK | | | |
| | | | TS | | | |
| ccaccccga | CCA CCC | 1321 | LEPGEKPYKCPECGKSFSSKKHL | 1870 | X₁X₂X₃X₄X₅X₆X₇X₈X₉CX₁₀X₁₁CX₁₂X₁₃X₁₄X₁₅X₁₆SKKHLAEHX₁₇X₁₈X₁₉ | 2419 |
| aggcccccg | CGA AGG | | AEHQRTHTGEKPYKCPECGKSFS | | HX₂₀XX₂₁X₂₂X₂₃X₂₄X₈X₉CX₁₀X₁₁CX₁₂X₁₃X₁₄X₁₅X₁₆RNDTLTEHX₁₇X₁₈ | |
| ccc | CCC CCG | | RNDTLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆SKKHLAEHX₁₇ | |
| | CCC | | GKSFSSKKHLAEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDHLTNH | |
| | | | KCPECGKSFSRSDHLTNHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QSGHLT | |
| | | | GEKPYKCPECGKSFSQSGHLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆SKKH | |
| | | | QRTHTGEKPYKCPECGKSFSSKK | | LAEHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | HLAEHQRTHTGEKPYKCPECGKS | | H5LTEHX₁₇X₁₃X₁₉HX₂₀X₂₁X₂₂X₂₃X₂₄X₂₅X₂₆X₂₇X₂₈X₂₉X₃₀ | |
| | | | FSTSHSLTEHQRTHTGEKPTGKK | | | |
| | | | TS | | | |
| ccactgcct | CCA CTG | 1322 | LEPGEKPYKCPECGKSFSDCRDL | 1871 | X₁X₂X₃X₄X₅X₆X₇X₈X₉CX₁₀X₁₁CX₁₂X₁₃X₁₄X₁₅X₁₆DCRDLARHX₁₇X₁₈X₁₉ | 2420 |
| cctcccagt | CCT CCT | | ARHQRTHTGEKPYKCPECGKSFS | | HX₂₀XX₂₀X₂₂X₂₃X₂₄X₈X₉CX₁₀X₁₁CX₁₂X₁₃X₁₄X₁₅X₁₆HRTTLTNHX₁₇X₁₈ | |
| gcc | CCC AGT | | HRTTLTNHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆SKKHLAEHX₁₇ | |
| | GCC | | GKSFSSKKHLAEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TKNSLTEH | |
| | | | KCPECGKSFSTKNSLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TKNSLT | |
| | | | GEKPYKCPECGKSFSTKNSLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆RNDA | |
| | | | QRTHTGEKPYKCPECGKSFSRND | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | ALTEHQRTHTGEKPYKCPECGKS | | HSLTEHX₁₇X₁₈X₁₉HX₂₀X₂₁X₂₂X₂₃X₂₄X₂₅X₂₆X₂₇X₂₈X₂₉X₃₀ | |
| | | | FSTSHSLTEHQRTHTGEKPTGKK | | | |
| | | | TS | | | |
| tgggaagat | TGG GAA | 1323 | LEPGEKPYKCPECGKSFSDPGAL | 1872 | X₁X₂X₃X₄X₅X₆X₇X₈X₉CX₁₀X₁₁CX₁₂X₁₃X₁₄X₁₅X₁₆DPGALVRHX₁₇X₁₈X₁₉ | 2421 |
| ctgctggga | GAT CTG | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QRAHLERHX₁₇X₁₈ | |
| gtc | CTG GGA | | QRAHLERHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RNDALTEHX₁₇ | |
| | GTC | | GKSFSRNDALTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RNDALTEH | |
| | | | KCPECGKSFSRNDALTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSGNLV | |
| | | | GEKPYKCPECGKSFSTSGNLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆QSSN | |
| | | | QRTHTGEKPYKCPECGKSFSQSS | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | NLVRHQRTHTGEKPYKCPECGKS | | DHLTTHX₁₇X₁₈X₁₉HX₂₀X₂₁X₂₂X₂₃X₂₄X₂₅X₂₆X₂₇X₂₈X₂₉X₃₀ | |
| | | | FSRSDHLTTHQRTHTGEKPTGKK | | | |
| | | | TS | | | |
| gctgggagt | GCT ggg | 1324 | LEPGEKPYKCPECGKSFSDCRDL | 1873 | X₁X₂X₃X₄X₅X₆X₇X₈X₉CX₁₀X₁₁CX₁₂X₁₃X₁₄X₁₅X₁₆DCRDLARHX₁₇X₁₈X₁₉ | 2422 |
| cttggccta | AGT CTT | | ARHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QNSTLTEHX₁₇X₁₈ | |
| gcc | GGC CTA | | QNSTLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DPGHLVRHX₁₇ | |
| | GCC | | GKSFSDPGHLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TTGALTEH | |
| | | | KCPECGKSFSTTGALTEHQRTHT | | X₁₇X₁₈X₁₉HX₂₀X₂₁X₂₂X₂₃X₂₄X₈X₉CXHX₁₁CX₁₂X₁₃X₁₄X₁₅X₁₆HRTTLT | |
| | | | GEKPYKCPECGKSFSHRTTLTNH | | NHX₁₇X₁₈X₁₉HX₂₀X₂₁X₂₂X₂₃X₂₄X₈X₉CX₁₀X₁₁CX₁₂X₁₃X₁₄X₁₅X₁₆RSDK | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | KLVRHQRTHTGEKPYKCPECGKS | | GELVRHX₁₇X₁₈X₁₉HX₂₀X₂₁X₂₂X₂₃X₂₄X₂₅X₂₆X₂₇X₂₈X₂₉X₃₀ | |
| | | | FSTSGELVRHQRTHTGEKPTGKK | | | |
| | | | TS | | | |
| gcctagcct | GCC TAG | 1325 | LEPGEKPYKCPECGKSFSTSGHL | 1874 | X₁X₂X₃X₄X₅X₆X₇X₈X₉CX₁₀X₁₁CX₁₂X₁₃X₁₄X₁₅X₁₆TSGHLVRHX₁₇X₁₈X₁₉ | 2423 |
| ctgtgaagg | CCT CTG | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDHLTNHX₁₇X₁₈ | |
| ggt | TGA AGG | | RSDHLTNHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QAGHLASHX₁₇ | |
| | GGT | | GKSFSQAGHLASHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RNDALTEH | |
| | | | KCPECGKSFSRNDALTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TKNSLT | |
| | | | GEKPYKCPECGKSFSTKNSLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆REDN | |
| | | | QRTHTGEKPYKCPECGKSFSRED | | LHTHX₁₇X₁₈X₁₉HX₂₀X₂₁X₂₂X₂₃X₂₄X₈X₉CX₁₀X₁₁CX₁₂X₁₃X₁₄X₁₅X₁₆DC | |
| | | | NLHTHQRTHTGEKPYKCPECGKS | | RDLARHX₁₇X₁₈X₁₉HX₂₀X₂₁X₂₂X₂₃X₂₄X₂₅X₂₆X₂₇X₂₈X₂₉X₃₀ | |
| | | | FSDCRDLARHQRTHTGEKPTGKK | | | |
| | | | TS | | | |
| tagcctctg | TAG CCT | 1326 | LEPGEKPYKCPECGKSFSQRAHL | 1875 | X₁X₂X₃X₄X₅X₆X₇X₈X₉CX₁₀X₁₁CX₁₂X₁₃X₁₄X₁₅X₁₆QRAHLERHX₁₇X₁₈X₁₉ | 2424 |
| tgaaggggt | CTG TGA | | ERHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSGHLVRHX₁₇X₁₈ | |
| gga | AGG GGT | | TSGHLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDHLTNHX₁₇ | |
| | GGA | | GKSFSRSDHLTNHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QAGHLASH | |
| | | | KCPECGKSFSQAGHLASHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RNDALT | |
| | | | GEKPYKCPECGKSFSRNDALTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆TKNS | |
| | | | QRTHTGEKPYKCPECGKSFSTKN | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆RE | |
| | | | SLTEHQRTHTGEKPYKCPECGKS | | DNLHTHX₁₇X₁₈X₁₉HX₂₀X₂₁X₂₂X₂₃X₂₄X₂₅X₂₆X₂₇X₂₈X₂₉X₃₀ | |
| | | | FSREDNLHTHQRTHTGEKPTGKK | | | |
| | | | TS | | | |
| cctctgtga | CCT CTG | 1327 | LEPGEKPYKCPECGKSFSDPGHL | 1876 | X₁X₂X₃X₄X₅X₆X₇X₈X₉CX₁₀X₁₁CX₁₂X₁₃X₁₄X₁₅X₁₆DPGHLVRHX₁₇X₁₈X₁₉ | 2425 |
| aggggtgga | TGA AGG | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QRAHLERHX₁₇X₁₈ | |
| ggc | GGT GGA | | QRAHLERHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSGHLVRHX₁₇ | |
| | GGC | | GKSFSTSGHLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDHLTNH | |
| | | | KCPECGKSFSRSDHLTNHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QAGHLA | |
| | | | GEKPYKCPECGKSFSQAGHLASH | | SHX₁₇X₁₈X₁₉HX₂₀X₂₁X₂₂X₂₃X₂₄X₈X₉CXHX₁₁CX₁₂X₁₃X₁₄X₁₅X₁₆RNDA | |
| | | | QRTHTGEKPYKCPECGKSFSRND | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆TK | |
| | | | ALTEHQRTHTGEKPYKCPECGKS | | NSLTEHX₁₇X₁₈X₁₉HX₂₀X₂₁X₂₂X₂₃X₂₄X₂₅X₂₆X₂₇X₂₈X₂₉X₃₀ | |
| | | | FSTKNSLTEHQRTHTGEKPTGKK | | | |
| | | | TS | | | |
| ggggtggag | GGG GTG | 1328 | LEPGEKPYKCPECGKSFSRSDKL | 1877 | X₁X₂X₃X₄X₅X₆X₇X₈X₉CX₁₀X₁₁CX₁₂X₁₃X₁₄X₁₅X₁₆RSDKLVRHX₁₇X₁₈X₁₉ | 2426 |
| gctctgccg | GAG GCT | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RNDTLTEHX₁₇X₁₈ | |
| ggg | CTG CCG | | RNDTLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RNDALTEHX₁₇ | |
| | GGG | | GKSFSRNDALTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSGELVRH | |
| | | | KCPECGKSFSTSGELVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDNLV | |
| | | | GEKPYKCPECGKSFSRSDNLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDE | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | ELVRHQRTHTGEKPYKCPECGKS | | DKLVRHX₁₇X₁₈X₁₉HX₂₀X₂₁X₂₂X₂₃X₂₄X₂₅X₂₆X₂₇X₂₈X₂₉X₃₀ | |
| | | | FSRSDKLVRHQRTHTGEKPTGKK | | | |
| | | | TS | | | |
| gaaggggtg | GAA GGG | 158 | LEPGEKPYKCPECGKSFSRNDTL | 159 | X₁X₂X₃X₄X₅X₆X₇X₈X₉CX₁₀X₁₁CX₁₂X₁₃X₁₄X₁₅X₁₆RNDTLTEHX₁₇X₁₈X₁₉ | 327 |
| gaggctctg | GTG GAG | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RNDALTEHX₁₇X₁₈ | |
| ccg | GCT CTG | | RNDALTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSGELVRHX₁₇ | |
| | CCG | | GKSFSTSGELVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDNLVRH | |
| | | | KCPECGKSFSRSDNLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDELV | |
| | | | GEKPYKCPECGKSFSRSDELVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDK | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆QS | |
| | | | KLVRHQRTHTGEKPYKCPECGKS | | SNLVRHX₁₇X₁₈X₁₉HX₂₀X₂₁X₂₂X₂₃X₂₄X₂₅X₂₆X₂₇X₂₈X₂₉X₃₀ | |
| | | | FSQSSNLVRHQRTHTGEKPTGKK | | | |
| | | | TS | | | |
| gtggaggct | GTG GAG | 1329 | LEPGEKPYKCPECGKSFSRSDHL | 1878 | X₁X₂X₃X₄X₅X₆X₇X₈X₉CX₁₀X₁₁CX₁₂X₁₃X₁₄X₁₅X₁₆RSDHLTNHX₁₇X₁₈X₁₉ | 2427 |
| ctgccgggg | GCT CTG | | TNHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDKLVRHX₁₇X₁₈ | |
| agg | CCG ggg | | RSDKLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RNDTLTEHX₁₇ | |
| | AGG | | GKSFSRNDTLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RNDALTEH | |
| | | | KCPECGKSFSRNDALTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSGELV | |
| | | | GEKPYKCPECGKSFSTSGELVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDN | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | NLVRHQRTHTGEKPYKCPECGKS | | DELVRHX₁₇X₁₈X₁₉HX₂₀X₂₁X₂₂X₂₃X₂₄X₂₅X₂₆X₂₇X₂₈X₂₉X₃₀ | |
| | | | FSRSDELVRHQRTHTGEKPTGKK | | | |
| | | | TS | | | |
| gaggctctg | GAG GCT | 1330 | LEPGEKPYKCPECGKSFSTSGHL | 1879 | X₁X₂X₃X₄X₅X₆X₇X₈X₉CX₁₀X₁₁CX₁₂X₁₃X₁₄X₁₅X₁₆TSGHLVRHX₁₇X₁₈X₁₉ | 2428 |
| ccggggagg | CTG CCG | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDHLTNHX₁₇X₁₈ | |
| ggt | ggg AGG | | RSDHLTNHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDKLVRHX₁₇ | |
| | GGT | | GKSFSRSDKLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RNDTLTEH | |
| | | | KCPECGKSFSRNDTLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RNDALT | |
| | | | GEKPYKCPECGKSFSRNDALTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆TSGE | |
| | | | QRTHTGEKPYKCPECGKSFSTSG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | ELVRHQRTHTGEKPYKCPECGKS | | DNLVRHX₁₇X₁₈X₁₉HX₂₀X₂₁X₂₂X₂₃X₂₄X₂₅X₂₆X₂₇X₂₈X₂₉X₃₀ | |
| | | | FSRSDNLVRHQRTHTGEKPTGKK | | | |
| | | | TS | | | |
| gctctgccg | GCT CTG | 1331 | LEPGEKPYKCPECGKSFSRSDKL | 1880 | X₁X₂X₃X₄X₅X₆X₇X₈X₉CX₁₀X₁₁CX₁₂X₁₃X₁₄X₁₅X₁₆RSDKLVRHX₁₇X₁₈X₁₉ | 2429 |
| gggaggggt | CCG ggg | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSGHLVRHX₁₇X₁₈ | |
| ggg | AGG GGT | | TSGHLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDHLTNHX₁₇ | |
| | GGG | | GKSFSRSDHLTNHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDKLVRH | |
| | | | KCPECGKSFSRSDKLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RNDTLT | |
| | | | GEKPYKCPECGKSFSRNDTLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆RNDA | |
| | | | QRTHTGEKPYKCPECGKSFSRND | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | ALTEHQRTHTGEKPYKCPECGKS | | GELVRHX₁₇X₁₈X₁₉HX₂₀X₂₁X₂₂X₂₃X₂₄X₂₅X₂₆X₂₇X₂₈X₂₉X₃₀ | |
| | | | FSTSGELVRHQRTHTGEKPTGKK | | | |
| | | | TS | | | |
| ctgccgggg | CTG CCG | 1332 | LEPGEKPYKCPECGKSFSTSGHL | 1881 | X₁X₂X₃X₄X₅X₆X₇X₈X₉CX₁₀X₁₁CX₁₂X₁₃X₁₄X₁₅X₁₆TSGHLVRHX₁₇X₁₈X₁₉ | 2430 |
| aggggtggg | ggg AGG | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDKLvRHX₁₇X₁₈ | |
| ggt | GGT GGG | | RSDKLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSGHLVRHX₁₇ | |
| | GGT | | GKSFSTSGHLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDHLTNH | |
| | | | KCPECGKSFSRSDHLTNHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDKLV | |
| | | | GEKPYKCPECGKSFSRSDKLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RNDT | |
| | | | QRTHTGEKPYKCPECGKSFSRND | | LTEHX₁₇X₁₈X₁₉HX₂₀X₂₁X₂₂X₂₃X₂₄X₈X₉CX₁₀X₁₁CX₁₂XEX₁₄X₁₅X₁₆RN | |
| | | | TLTEHQRTHTGEKPYKCPECGKS | | DALTEHX₁₇X₁₈X₁₉HX₂₀X₂₁X₂₂X₂₃X₂₄X₂₅X₂₆X₂₇X₂₈X₂₉X₃₀ | |
| | | | FSRNDALTEHQRTHTGEKPTGKK | | | |
| | | | TS | | | |
| cggggaggg | CGG GGA | 1333 | LEPGEKPYKCPECGKSFSTTGNL | 1882 | X₁X₂X₃X₄X₅X₆X₇X₈X₉CX₁₀X₁₁CX₁₂X₁₃X₁₄X₁₅X₁₆TTGNLTVHX₁₇X₁₈X₁₉ | 2431 |
| gtgggggtt | GGG GTG | | TVHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSGSLVRHX₁₇X₁₈ | |
| aat | GGG GTT | | TSGSLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDKLVRHX₁₇ | |
| | AAT | | GKSFSRSDKLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDELVRH | |
| | | | KCPECGKSFSRSDELVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDKLV | |
| | | | GEKPYKCPECGKSFSRSDKLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆QRAH | |
| | | | QRTHTGEKPYKCPECGKSFSQRA | | LERHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | HLERHQRTHTGEKPYKCPECGKS | | DKLTEHX₁₇X₁₈X₁₉HX₂₀X₂₁X₂₂X₂₃X₂₄X₂₅X₂₆X₂₇X₂₈X₂₉X₃₀ | |
| | | | FSRSDKLTEHQRTHTGEKPTGKK | | | |
| | | | TS | | | |
| ggaggggtg | GGA GGG | 154 | LEPGEKPYKCPECGKSFSTSGHL | 155 | X₁X₂X₃X₄X₅X₆X₇X₈X₉CX₁₀X₁₁CX₁₂X₁₃X₁₄X₁₅X₁₆TSGHLVRHX₁₇X₁₈X₁₉ | 326 |
| ggggttaat | GTG GGG | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TTGNLTVHX₁₇X₁₈ | |
| ggt | GTT AAT | | TTGNLTVHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSGSLVRHX₁₇ | |
| | GGT | | GKSFSTSGSLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDKLVRH | |
| | | | KCPECGKSFSRSDKLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDELV | |
| | | | GEKPYKCPECGKSFSRSDELVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDK | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆QR | |
| | | | KLVRHQRTHTGEKPYKCPECGKS | | AHLERHX₁₇X₁₈X₁₉HX₂₀X₂₁X₂₂X₂₃X₂₄X₂₅X₂₆X₂₇X₂₈X₂₉X₃₀ | |
| | | | FSQRAHLERHQRTHTGEKPTGKK | | | |
| | | | TS | | | |
| ggcggggct | GGC GGG | 1334 | LEPGEKPYKCPECGKSFSRSDHL | 1883 | X₁X₂X₃X₄X₅X₆X₇X₈X₉CX₁₀X₁₁CX₁₂X₁₃X₁₄X₁₅X₁₆RSDHLTTHX₁₇X₁₈X₁₉ | 2432 |
| gcagggatt | GCT GCA | | TTHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆HKNALQNHX₁₇X₁₈ | |
| tgg | ggg ATT | | HKNALQNHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDKLVRHX₁₇ | |
| | TGG | | GKSFSRSDKLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QSGDLRRH | |
| | | | KCPECGKSFSQSGDLRRHQRTHT | | X₁₇X₁₈X₁₉HX₂₀X₂₁X₂₂X₂₃X₂₄X₈X₉CXHX₁₁CX₁₂X₁₃X₁₄X₁₅X₁₆TSGELV | |
| | | | GEKPYKCPECGKSFSTSGELVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDK | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆DP | |
| | | | KLVRHQRTHTGEKPYKCPECGKS | | GHLVRHX₁₇X₁₈X₁₉HX₂₀X₂₁X₂₂X₂₃X₂₄X₂₅X₂₆X₂₇X₂₈X₂₉X₃₀ | |
| | | | FSDPGHLVRHQRTHTGEKPTGKK | | | |
| | | | TS | | | |
| ctgggcggg | CTG GGC | 1335 | LEPGEKPYKCPECGKSFSHKNAL | 1884 | X₁X₂X₃X₄X₅X₆X₇X₈X₉CX₁₀X₁₁CX₁₂X₁₃X₁₄X₁₅X₁₆HKNALQNHX₁₇X₁₈X₁₉ | 2433 |
| gctgcaggg | GGG GCT | | QNHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDKLVRHX₁₇X₁₈ | |
| att | GCA ggg | | RSDKLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QSGDLRRHX₁₇ | |
| | ATT | | GKSFSQSGDLRRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSGELVRH | |
| | | | KCPECGKSFSTSGELVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDKLV | |
| | | | GEKPYKCPECGKSFSRSDKLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆DPGH | |
| | | | QRTHTGEKPYKCPECGKSFSDPG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RN | |
| | | | HLVRHQRTHTGEKPYKCPECGKS | | DALTEHX₁₇X₁₃X₁₉HX₂₀X₂₁X₂₂X₂₃X₂₄X₂₅X₂₆X₂₇X₂₈X₂₉X₃₀ | |
| | | | FSRNDALTEHQRTHTGEKPTGKK | | | |
| | | | TS | | | |
| ggggctgca | GGG GCT | 1336 | LEPGEKPYKCPECGKSFSRNDAL | 1885 | X₁X₂X₃X₄X₅X₆X₇X₈X₉CX₁₀X₁₁CX₁₂X₁₃X₁₄X₁₅X₁₆RNDALTEHX₁₇X₁₈X₁₉ | 2434 |
| gggatttgg | GCA ggg | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDHLTTHX₁₇X₁₈ | |
| ctg | ATT TGG | | RSDHLTTHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆HKNALQNHX₁₇ | |
| | CTG | | GKSFSHKNALQNHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDKLVRH | |
| | | | KCPECGKSFSRSDKLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QSGDDR | |
| | | | GEKPYKCPECGKSFSQSGDLRRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆TSGE | |
| | | | QRTHTGEKPYKCPECGKSFSTSG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | ELVRHQRTHTGEKPYKCPECGKS | | DKLVRHX₁₇X₁₃X₁₉HX₂₀X₂₁X₂₂X₂₃X₂₄X₂₅X₂₆X₂₇X₂₈X₂₉X₃₀ | |
| | | | FSRSDKLVRHQRTHTGEKPTGKK | | | |
| | | | TS | | | |
| aggctgggc | AGG CTG | 150 | LEPGEKPYKCPECGKSFSRSDKL | 151 | X₁X₂X₃X₄X₅X₆X₇X₈X₉CX₁₀X₁₁CX₁₂X₁₃X₁₄X₁₅X₁₆RSDKLVRHX₁₇X₁₈X₁₉ | 325 |
| ggggctgca | GGC GGG | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QSGDLRRHX₁₇X₁₈ | |
| ggg | GCT GCA | | QSGDLRRHQRTHTGEKPYKCPEC | | X₁₉HX₂₀X₂₁X₂₂X₂₃X₂₄X₈X₉CX₁₀X₁₁CX₁₂X₁₃XHX₁₅X₁₆TSGELVRHX₁₇ | |
| | GGG | | GKSFSTSGELVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDKLVRH | |
| | | | KCPECGKSFSRSDKLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DPGHLV | |
| | | | GEKPYKCPECGKSFSDPGHLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RNDA | |
| | | | QRTHTGEKPYKCPECGKSFSRND | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | ALTEHQRTHTGEKPYKCPECGKS | | DHLTNHX₁₇X₁₈X₁₉HX₂₀X₂₁X₂₂X₂₃X₂₄X₂₅X₂₆X₂₇X₂₈X₂₉X₃₀ | |
| | | | FSRSDHLTNHQRTHTGEKPTGKK | | | |
| | | | TS | | | |
| gtggatagg | GTG GAT | 1337 | LEPGEKPYKCPECGKSFSTSGEL | 1886 | X₁X₂X₃X₄X₅X₆X₇X₈X₉CX₁₀X₁₁CX₁₂X₁₃X₁₄X₁₅X₁₆TSGELVRHX₁₇X₁₈X₁₉ | 2435 |
| ctgggcggg | AGG CTG | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDKLVRHX₁₇X₁₈ | |
| gct | GGC GGG | | RSDKLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DPGHLVRHX₁₇ | |
| | GCT | | GKSFSDPGHLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RNDALTEH | |
| | | | KCPECGKSFSRNDALTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDHLT | |
| | | | GEKPYKCPECGKSFSRSDHLTNH | | NHX₁₇X₁₈X₁₉HX₂₀X₂₁X₂₂X₂₃X₂₄X₈X₉CX₁₀X₁₁CX₁₂X₁₃X₁₄X₁₅X₁₆TSGN | |
| | | | QRTHTGEKPYKCPECGKSFSTSG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | NLVRHQRTHTGEKPYKCPECGKS | | DELVRHX₁₇X₁₈X₁₉HX₂₀X₂₁X₂₂X₂₃X₂₄X₂₅X₂₆X₂₇X₂₈X₂₉X₃₀ | |
| | | | FSRSDELVRHQRTHTGEKPTGKK | | | |
| | | | TS | | | |
| gataggctg | GAT AGG | 1338 | LEPGEKPYKCPECGKSFSQSGDL | 1887 | X₁X₂X₃X₄X₅X₆X₇X₈X₉CX₁₀X₁₁CX₁₂X₁₃X₁₄X₁₅X₁₆QSGDLRRHX₁₇X₁₈X₁₉ | 2436 |
| ggcggggct | CTG GGC | | RRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSGELVRHX₁₇X₁₈ | |
| gca | GGG GCT | | TSGELVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDKLVRHX₁₇ | |
| | GCA | | GKSFSRSDKLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DPGHLVRH | |
| | | | KCPECGKSFSDPGHLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RNDALT | |
| | | | GEKPYKCPECGKSFSRNDALTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆RSDH | |
| | | | QRTHTGEKPYKCPECGKSFSRSD | | LTNHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | HLTNHQRTHTGEKPYKCPECGKS | | GNLVRHX₁₇X₁₃X₁₉HX₂₀X₂₁X₂₂X₂₃X₂₄X₂₅X₂₆X₂₇X₂₈X₂₉X₃₀ | |
| | | | FSTSGNLVRHQRTHTGEKPTGKK | | | |
| | | | TS | | | |
| ccggtggat | CCG GTG | 1339 | LEPGEKPYKCPECGKSFSRSDKL | 1888 | X₁X₂X₃X₄X₅X₆X₇X₈X₉CX₁₀X₁₁CX₁₂X₁₃X₁₄X₁₅X₁₆RSDKLVRHX₁₇X₁₈X₁₉ | 2437 |
| aggctgggc | GAT AGG | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆DPGHLVRHX₁₇X₁₈ | |
| ggg | CTG GGC | | DPGHLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RNDALTEHX₁₇ | |
| | GGG | | GKSFSRNDALTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDHLTNH | |
| | | | KCPECGKSFSRSDHLTNHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSGNLV | |
| | | | GEKPYKCPECGKSFSTSGNLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDE | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RN | |
| | | | ELVRHQRTHTGEKPYKCPECGKS | | DTLTEHX₁₇X₁₃X₁₉HX₂₀X₂₁X₂₂X₂₃X₂₄X₂₅X₂₆X₂₇X₂₈X₂₉X₃₀ | |
| | | | FSRNDTLTEHQRTHTGEKPTGKK | | | |
| | | | TS | | | |
| ccgccggtg | CCG CCG | 1340 | LEPGEKPYKCPECGKSFSDPGHL | 1889 | X₁X₂X₃X₄X₅X₆X₇X₈X₉CX₁₀X₁₁CX₁₂X₁₃X₁₄X₁₅X₁₆DPGHLVRHX₁₇X₁₈X₁₉ | 2438 |
| gataggctg | GTG GAT | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RNDALTEHX₁₇X₁₈ | |
| ggc | AGG CTG | | RNDALTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDHLTNHX₁₇ | |
| | GGC | | GKSFSRSDHLTNHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSGNLVRH | |
| | | | KCPECGKSFSTSGNLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDELV | |
| | | | GEKPYKCPECGKSFSRSDELVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RNDT | |
| | | | QRTHTGEKPYKCPECGKSFSRND | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆RN | |
| | | | TLTEHQRTHTGEKPYKCPECGKS | | DTLTEHX₁₇X₁₈X₁₉HX₂₀X₂₁X₂₂X₂₃X₂₄X₂₅X₂₆X₂₇X₂₈X₂₉X₃₀ | |
| | | | FSRNDTLTEHQRTHTGEKPTGKK | | | |
| | | | TS | | | |
| cccccgccg | CCC CCG | 1341 | LEPGEKPYKCPECGKSFSRNDAL | 1890 | X₁X₂X₃X₄X₅X₆X₇X₈X₉CX₁₀X₁₁CX₁₂X₁₃X₁₄X₁₅X₁₆RNDALTEHX₁₇X₁₈X₁₉ | 2439 |
| gtggatagg | CCG GTG | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDHLTNHX₁₇X₁₈ | |
| ctg | GAT AGG | | RSDHLTNHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSGNLVRHX₁₇ | |
| | CTG | | GKSFSTSGNLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDELVRH | |
| | | | KCPECGKSFSRSDELVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RNDTLT | |
| | | | GEKPYKCPECGKSFSRNDTLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆RNDT | |
| | | | QRTHTGEKPYKCPECGKSFSRND | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆SK | |
| | | | TLTEHQRTHTGEKPYKCPECGKS | | KHLAEHX₁₇X₁₈X₁₉HX₂₀X₂₁X₂₂X₂₃X₂₄X₂₅X₂₆X₂₇X₂₈X₂₉X₃₀ | |
| | | | FSSKKHLAEHQRTHTGEKPTGKK | | | |
| | | | TS | | | |
| ggtcccccg | GGT CCC | 1342 | LEPGEKPYKCPECGKSFSRSDHL | 1891 | X₁X₂X₃X₄X₅X₆X₇X₈X₉CX₁₀X₁₁CX₁₂X₁₃X₁₄X₁₅X₁₆RSDHLTNHX₁₇X₁₈X₁₉ | 2440 |
| ccggtggat | CCG CCG | | TNHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSGNLVRHX₁₇X₁₈ | |
| agg | GTG GAT | | TSGNLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDELVRHX₁₇ | |
| | AGG | | GKSFSRSDELVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RNDTLTEH | |
| | | | KCPECGKSFSRNDTLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RNDTLT | |
| | | | GEKPYKCPECGKSFSRNDTLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆SKKH | |
| | | | QRTHTGEKPYKCPECGKSFSSKK | | LAEHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | HLAEHQRTHTGEKPYKCPECGKS | | GHLVRHX₁₇X₁₈X₁₉HX₂₀X₂₁X₂₂X₂₃X₂₄X₂₅X₂₆X₂₇X₂₈X₂₉X₃₀ | |
| | | | FSTSGHLVRHQRTHTGEKPTGKK | | | |
| | | | TS | | | |
| ataggctgg | ATA GGC | 1343 | LEPGEKPYKCPECGKSFSRADNL | 1892 | X₁X₂X₃X₄X₅X₆X₇X₈X₉CX₁₀X₁₁CX₁₂X₁₃X₁₄X₁₅X₁₆RADNLTEHX₁₇X₁₈X₁₉ | 2441 |
| gcggggctg | TGG GCG | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RNDALTEHX₁₇X₁₈ | |
| cag | ggg CTG | | RNDALTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDKLVRHX₁₇ | |
| | CAG | | GKSFSRSDKLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDDLVRH | |
| | | | KCPECGKSFSRSDDLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDHLT | |
| | | | GEKPYKCPECGKSFSRSDHLTTH | | THX₁₇X₁₈X₁₉HX₂₀X₂₁X₂₂X₂₃X₂₄X₈X₉CX₁₀X₁₁CX₁₂X₁₃X₁₄X₁₅X₁₆DPGH | |
| | | | QRTHTGEKPYKCPECGKSFSDPG | | LVRHX₁₇X₁₈X₁₉HX₂₀X₂₁X₂₂X₂₃X₂₄X₈X₉CX₁₀X₁₁CX₁₂XEX₁₄X₁₅X₁₆QK | |
| | | | HLVRHQRTHTGEKPYKCPECGKS | | SSLIAHX₁₇X₁₈X₁₉HX₂₀X₂₁X₂₂X₂₃X₂₄X₂₅X₂₆X₂₇X₂₈X₂₉X₃₀ | |
| | | | FSQKSSLIAHQRTHTGEKPTGKK | | | |
| | | | TS | | | |
| cggtggata | CGG TGG | 1344 | LEPGEKPYKCPECGKSFSRSDKL | 1893 | X₁X₂X₃X₄X₅X₆X₇X₈X₉CX₁₀X₁₁CX₁₂X₁₃X₁₄X₁₅X₁₆RSDKLVRHX₁₇X₁₈X₁₉ | 2442 |
| ggctgggcg | ATA GGC | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDDLVRHX₁₇X₁₈ | |
| ggg | TGG GCG | | RSDDLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDHLTTHX₁₇ | |
| | GGG | | GKSFSRSDHLTTHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DPGHLVRH | |
| | | | KCPECGKSFSDPGHLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QKSSLI | |
| | | | GEKPYKCPECGKSFSQKSSLIAH | | AHX₁₇X₁₈X₁₉HX₂₀X₂₁X₂₂X₂₃X₂₄X₈X₉CX₁₀X₁₁CX₁₂X₁₃X₁₄X₁₅X₁₆RSDH | |
| | | | QRTHTGEKPYKCPECGKSFSRSD | | LTTHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | HLTTHQRTHTGEKPYKCPECGKS | | DKLTEHX₁₇X₁₈X₁₉HX₂₀X₂₁X₂₂X₂₃X₂₄X₂₅X₂₆X₂₇X₂₈X₂₉X₃₀ | |
| | | | FSRSDKLTEHQRTHTGEKPTGKK | | | |
| | | | TS | | | |
| tggataggc | TGG ATA | 1345 | LEPGEKPYKCPECGKSFSRNDAL | 1894 | X₁X₂X₃X₄X₅X₆X₇X₈X₉CX₁₀X₁₁CX₁₂X₁₃X₁₄X₁₅X₁₆RNDALTEHX₁₇X₁₈X₁₉ | 2443 |
| tgggcgggg | GGC TGG | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDKLVRHX₁₇X₁₈ | |
| ctg | GCG ggg | | RSDKLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDDLVRHX₁₇ | |
| | CTG | | GKSFSRSDDLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDHLTTH | |
| | | | KCPECGKSFSRSDHLTTHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DPGHLV | |
| | | | GEKPYKCPECGKSFSDPGHLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆QKSS | |
| | | | QRTHTGEKPYKCPECGKSFSQKS | | LIAHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | SLIAHQRTHTGEKPYKCPECGKS | | DHLTTHX₁₇X₁₈X₁₉HX₂₀X₂₁X₂₂X₂₃X₂₄X₂₅X₂₆X₂₇X₂₈X₂₉X₃₀ | |
| | | | FSRSDHLTTHQRTHTGEKPTGKK | | | |
| | | | TS | | | |
| cgccggtgg | CGC CGG | 1346 | LEPGEKPYKCPECGKSFSRSDDL | 1895 | X₁X₂X₃X₄X₅X₆X₇X₈X₉CX₁₀X₁₁CX₁₂X₁₃X₁₄X₁₅X₁₆RSDDLVRHX₁₇X₁₈X₁₉ | 2444 |
| ataggctgg | TGG ATA | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDHLTTHX₁₇X₁₈ | |
| gcg | GGC TGG | | RSDHLTTHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DPGHLVRHX₁₇ | |
| | GCG | | GKSFSDPGHLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QKSSLIAH | |
| | | | KCPECGKSFSQKSSLIAHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDHLT | |
| | | | GEKPYKCPECGKSFSRSDHLTTH | | THX₁₇X₁₈X₁₉HX₂₀X₂₁X₂₂X₂₃X₂₄X₈X₉CX₁₀X₁₁CX₁₂X₁₃X₁₄X₁₅X₁₆RSDK | |
| | | | QRTHTGEKPYKCPECGKSFSRSD | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆HT | |
| | | | KLTEHQRTHTGEKPYKCPECGKS | | GHLLEHX₁₇X₁₈X₁₉HX₂₀X₂₁X₂₂X₂₃X₂₄X₂₅X₂₆X₂₇X₂₈X₂₉X₃₀ | |
| | | | FSHTGHLLEHQRTHTGEKPTGKK | | | |
| | | | TS | | | |
| gtcccccgc | GTC CCC | 1347 | LEPGEKPYKCPECGKSFSDPGHL | 1896 | X₁X₂X₃X₄X₅X₆X₇X₈X₉CX₁₀X₁₁CX₁₂X₁₃X₁₄X₁₅X₁₆DPGHLVRHX₁₇X₁₈X₁₉ | 2445 |
| cggtggata | CGC CGG | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QKSSLIAHX₁₇X₁₈ | |
| ggc | TGG ATA | | QKSSLIAHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDHLTTHX₁₇ | |
| | GGC | | GKSFSRSDHLTTHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDKLTEH | |
| | | | KCPECGKSFSRSDKLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆HTGHLL | |
| | | | GEKPYKCPECGKSFSHTGHLLEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆SKKH | |
| | | | QRTHTGEKPYKCPECGKSFSSKK | | LAEHX₁₇X₁₈X₁₉HX₂₀X₂₁X₂₂X₂₃X₂₄X₈X₉CX₁₀X₁₁CX₁₂X₁₃X₁₄X₁₅X₁₆DP | |
| | | | HLAEHQRTHTGEKPYKCPECGKS | | GALVRHX₁₇X₁₈X₁₉HX₂₀X₂₁X₂₂X₂₃X₂₄X₂₅X₂₆X₂₇X₂₈X₂₉X₃₀ | |
| | | | FSDPGALVRHQRTHTGEKPTGKK | | | |
| | | | TS | | | |
| ccccgccgg | CCC CGC | 1348 | LEPGEKPYKCPECGKSFSRSDHL | 1897 | X₁X₂X₃X₄X₅X₆X₇X₈X₉CXₗ₀X₁₁CX₁₂X₁₃X₁₄X₁₅X₁₆RSDHLTTHX₁₇XHX₁₉ | 2446 |
| tggataggc | CGG TGG | | TTHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆DPGHLVRHX₁₇X₁₈ | |
| tgg | ATA GGC | | DPGHLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QKSSLIAHX₁₇ | |
| | TGG | | GKSFSQKSSLIAHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDHLTTH | |
| | | | KCPECGKSFSRSDHLTTHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDKLT | |
| | | | GEKPYKCPECGKSFSRSDKLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆HTGH | |
| | | | QRTHTGEKPYKCPECGKSFSHTG | | LLEHX₁₇X₁₈X₁₉HX₂₀X₂₁X₂₂X₂₃X₂₄X₈X₉CX₁₀X₁₁CX₁₂X₁₃X₁₄X₁₅X₁₆SK | |
| | | | HLLEHQRTHTGEKPYKCPECGKS | | KHLAEHX₁₇X₁₈X₁₉HX₂₀X₂₁X₂₂X₂₃X₂₄X₂₅X₂₆X₂₇X₂₈X₂₉X₃₀ | |
| | | | FSSKKHLAEHQRTHTGEKPTGKK | | | |
| | | | TS | | | |
| ggctgggcg | GGC TGG | 1349 | LEPGEKPYKCPECGKSFSQRAHL | 1898 | X₁X₂X₃X₄X₅X₆X₇X₈X₉CX₁₀X₁₁CX₁₂X₁₃X₁₄X₁₅X₁₆QRAHLERHX₁₇X₁₈X₁₉ | 2447 |
| gggctgcag | GCG ggg | | ERHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RADNLTEHX₁₇X₁₈ | |
| gga | CTG CAG | | RADNLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RNDALTEHX₁₇ | |
| | GGA | | GKSFSRNDALTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDKLVRH | |
| | | | KCPECGKSFSRSDKLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDDLV | |
| | | | GEKPYKCPECGKSFSRSDDLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDH | |
| | | | QRTHTGEKPYKCPECGKSFSRSD | | LTTHX₁₇X₁₈X₁₉HX₂₀X₂₁X₂₂X₂₃X₂₄X₈X₉CX₁₀X₁₁CX₁₂X₁₃X₁₄X₁₅X₁₆DP | |
| | | | HLTTHQRTHTGEKPYKCPECGKS | | GHLVRHX₁₇X₁₈X₁₉HX₂₀X₂₁X₂₂X₂₃X₂₄X₂₅X₂₆X₂₇X₂₈X₂₉X₃₀ | |
| | | | FSDPGHLVRHQRTHTGEKPTGKK | | | |
| | | | TS | | | |
| ggtggatag | GGT GGA | 1350 | LEPGEKPYKCPECGKSFSDPGHL | 1899 | X₁X₂X₃X₄X₅X₆X₇X₈X₉CX₁₀X₁₁CX₁₂X₁₃X₁₄X₁₅X₁₆DPGHLVRHX₁₇X₁₈X₁₉ | 2448 |
| gctgggcgg | TAG GCT | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDKLTEHX₁₇X₁₈ | |
| ggc | ggg CGG | | RSDKLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDKLVRHX₁₇ | |
| | GGC | | GKSFSRSDKLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSGELVRH | |
| | | | KCPECGKSFSTSGELVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆REDNLH | |
| | | | GEKPYKCPECGKSFSREDNLHTH | | THX₁₇X₁₈X₁₉HX₂₀X₂₁X₂₂X₂₃X₂₄X₈X₉CX₁₀X₁₁CX₁₂X₁₃X₁₄X₁₅X₁₆QRAH | |
| | | | QRTHTGEKPYKCPECGKSFSQRA | | LERHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | HLERHQRTHTGEKPYKCPECGKS | | GHLVRHX₁₇X₁₃X₁₉HX₂₀X₂₁X₂₂X₂₃X₂₄X₂₅X₂₆X₂₇X₂₈X₂₉X₃₀ | |
| | | | FSTSGHLVRHQRTHTGEKPTGKK | | | |
| | | | TS | | | |
| gccggtgga | GCC GGT | 1351 | LEPGEKPYKCPECGKSFSRSDKL | 1900 | X₁X₂X₃X₄X₅X₆X₇X₈X₉CX₁₀X₁₁CX₁₂X₁₃X₁₄X₁₅X₁₆RSDKLTEHX₁₇X₁₈X₁₉ | 2449 |
| taggctggg | GGA TAG | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDKLVRHX₁₇X₁₈ | |
| cgg | GCT ggg | | RSDKLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSGELVRHX₁₇ | |
| | CGG | | GKSFSTSGELVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆REDNLHTH | |
| | | | KCPECGKSFSREDNLHTHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QRAHLE | |
| | | | GEKPYKCPECGKSFSQRAHLERH | | RHX₁₇X₁₈X₁₉HX₂₀X₂₁X₂₂X₂₃X₂₄X₈X₉CX₁₀X₁₁CX₁₂X₁₃X₁₄X₁₅X₁₆TSGH | |
| | | | QRTHTGEKPYKCPECGKSFSTSG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆DC | |
| | | | HLVRHQRTHTGEKPYKCPECGKS | | RDLARHX₁₇X₁₈X₁₉HX₂₀X₂₁X₂₂X₂₃X₂₄X₂₅X₂₆X₂₇X₂₈X₂₉X₃₀ | |
| | | | FSDCRDLARHQRTHTGEKPTGKK | | | |
| | | | TS | | | |
| cccgccggt | CCC GCC | 1352 | LEPGEKPYKCPECGKSFSRSDKL | 1901 | X₁X₂X₃X₄X₅X₆X₇X₈X₉CX₁₀X₁₁CX₁₂X₁₃X₁₄X₁₅X₁₆RSDKLVRHX₁₇X₁₈X₁₉ | 2450 |
| ggataggct | GGT GGA | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSGELVRHX₁₇X₁₈ | |
| ggg | TAG GCT | | TSGELVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆REDNLHTHX₁₇ | |
| | GGG | | GKSFSREDNLHTHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QRAHLERH | |
| | | | KCPECGKSFSQRAHLERHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSGHLV | |
| | | | GEKPYKCPECGKSFSTSGHLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆DCRD | |
| | | | QRTHTGEKPYKCPECGKSFSDCR | | LARHX₁₇X₁₈X₁₉HX₂₀X₂₁X₂₂X₂₃X₂₄X₈X₉CX₁₀X₁₁CX₁₂X₁₃X₁₄X₁₅X₁₆SK | |
| | | | DLARHQRTHTGEKPYKCPECGKS | | KHLAEHX₁₇X₁₈X₁₉HX₂₀X₂₁X₂₂X₂₃X₂₄X₂₅X₂₆X₂₇X₂₈X₂₉X₃₀ | |
| | | | FSSKKHLAEHQRTHTGEKPTGKK | | | |
| | | | TS | | | |
| gctgacacc | GCT GAC | 1353 | LEPGEKPYKCPECGKSFSTTGNL | 1902 | X₁X₂X₃X₄X₅X₆X₇X₈X₉CX₁₀X₁₁CX₁₂X₁₃X₁₄X₁₅X₁₆TTGNLTVHX₁₇X₁₈X₁₉ | 2451 |
| cggggtgct | ACC CGG | | TVHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSGELVRHX₁₇X₁₈ | |
| aat | GGT GCT | | TSGELVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSGHLVRHX₁₇ | |
| | AAT | | GKSFSTSGHLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDKLTEH | |
| | | | KCPECGKSFSRSDKLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DKKDLT | |
| | | | GEKPYKCPECGKSFSDKKDLTRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆DPGN | |
| | | | QRTHTGEKPYKCPECGKSFSDPG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | NLVRHQRTHTGEKPYKCPECGKS | | GELVRHX₁₇X₁₈X₁₉HX₂₀X₂₁X₂₂X₂₃X₂₄X₂₅X₂₆X₂₇X₂₈X₂₉X₃₀ | |
| | | | FSTSGELVRHQRTHTGEKPTGKK | | | |
| | | | TS | | | |
| ctggctgac | CTG GCT | 1354 | LEPGEKPYKCPECGKSFSTSGEL | 1903 | X₁X₂X₃X₄X₅X₆X₇X₈X₉CX₁₀X₁₁CX₁₂X₁₃X₁₄X₁₅X₁₆TSGELVRHX₁₇X₁₈X₁₉ | 2452 |
| acccggggt | GAC ACC | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSGHLVRHX₁₇X₁₈ | |
| gct | CGG GGT | | TSGHLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDKLTEHX₁₇ | |
| | GCT | | GKSFSRSDKLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DKKDLTRH | |
| | | | KCPECGKSFSDKKDLTRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DPGNLV | |
| | | | GEKPYKCPECGKSFSDPGNLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆TSGE | |
| | | | QRTHTGEKPYKCPECGKSFSTSG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RN | |
| | | | ELVRHQRTHTGEKPYKCPECGKS | | DALTEHX₁₇X₁₈X₁₉HX₂₀X₂₁X₂₂X₂₃X₂₄X₂₅X₂₆X₂₇X₂₈X₂₉X₃₀ | |
| | | | FSRNDALTEHQRTHTGEKPTGKK | | | |
| | | | TS | | | |
| acaactgct | ACA ACT | 1355 | LEPGEKPYKCPECGKSFSTHLDL | 1904 | X₁X₂X₃X₄X₅X₆X₇X₈X₉CX₁₀X₁₁CX₁₂X₁₃X₁₄X₁₅X₁₆THLDLIRHX₁₇X₁₈X₁₉ | 2453 |
| ggggcccta | GCT GGG | | IRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QNSTLTEHX₁₇X₁₈ | |
| act | GCC CTA | | QNSTLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DCRDLARHX₁₇ | |
| | ACT | | GKSFSDCRDLARHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDKLVRH | |
| | | | KCPECGKSFSRSDKLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSGELV | |
| | | | GEKPYKCPECGKSFSTSGELVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆THLD | |
| | | | QRTHTGEKPYKCPECGKSFSTHL | | LIRHX₁₇X₁₈X₁₉HX₂₀X₂₁X₂₂X₂₃X₂₄X₈X₉CX₁₀X₁₁CX₁₂X₁₃X₁₄X₁₅X₁₆SP | |
| | | | DLIRHQRTHTGEKPYKCPECGKS | | ADLTRHX₁₇X₁₈X₁₉HX₂₀X₂₁X₂₂X₂₃X₂₄X₂₅X₂₆X₂₇X₂₈X₂₉X₃₀ | |
| | | | FSSPADLTRHQRTHTGEKPTGKK | | | |
| | | | TS | | | |
| ctaattaca | CTA ATT | 1356 | LEPGEKPYKCPECGKSFSDCRDL | 1905 | X₁X₂X₃X₄X₅X₆X₇X₈X₉CX₁₀X₁₁CX₁₂X₁₃X₁₄X₁₅X₁₆DCRDLARHX₁₇X₁₈X₁₉ | 2454 |
| actgctggg | ACA ACT | | ARHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDKLVRHX₁₇X₁₈ | |
| gcc | GCT GGG | | RSDKLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSGELVRHX₁₇ | |
| | GCC | | GKSFSTSGELVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆THLDLIRH | |
| | | | KCPECGKSFSTHLDLIRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆SPADLT | |
| | | | GEKPYKCPECGKSFSSPADLTRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆HKNA | |
| | | | QRTHTGEKPYKCPECGKSFSHKN | | LQNHX₁₇X₁₈X₁₉HX₂₀X₂₁X₂₂X₂₃X₂₄X₈X₉CX₁₀X₁₁CX₁₂X₁₃X₁₄X₁₅X₁₆QN | |
| | | | ALQNHQRTHTGEKPYKCPECGKS | | STLTEHX₁₇EHX₁₇X₁₈X₁₉HX₂₀X₂₁X₂₂X₂₃X₂₄X₂₅X₂₆X₂₇X₂₈X₂₉X₃₀ | |
| | | | FSQNSTLTEHQRTHTGEKPTGKK | | | |
| | | | TS | | | |
| attacaact | ATT ACA | 1357 | LEPGEKPYKCPECGKSFSQNSTL | 1906 | X₁X₂X₃X₄X₅X₆X₇X₈X₉CX₁₀X₁₁CX₁₂X₁₃X₁₄X₁₅X₁₆QNSTLTEHX₁₇X₁₈X₁₉ | 2455 |
| gctggggcc | ACT GCT | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆DCRDLARHX₁₇X₁₈ | |
| cta | GGG GCC | | DCRDLARHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDKLVRHX₁₇ | |
| | CTA | | GKSFSRSDKLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSGELVRH | |
| | | | KCPECGKSFSTSGELVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆THLDLI | |
| | | | GEKPYKCPECGKSFSTHLDLIRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆SPAD | |
| | | | QRTHTGEKPYKCPECGKSFSSPA | | LTRHX₁₇X₁₈X₁₉HX₂₀X₂₁X₂₂X₂₃X₂₄X₈X₉CX₁₀X₁₁CX₁₂X₁₃X₁₄X₁₅X₁₆HK | |
| | | | DLTRHQRTHTGEKPYKCPECGKS | | NALQNHX₁₇X₁₈X₁₉HX₂₀X₂₁X₂₂X₂₃X₂₄X₂₅X₂₆X₂₇X₂₈X₂₉X₃₀ | |
| | | | FSHKNALQNHQRTHTGEKPTGKK | | | |
| | | | TS | | | |
| gtgctaatt | gtg CTA | 1358 | LEPGEKPYKCPECGKSFSRSDKL | 1907 | X₁X₂X₃X₄X₅X₆X₇X₈X₉CX₁₀X₁₁CX₁₂X₁₃X₁₄X₁₅X₁₆RSDKLVRHX₁₇X₁₈X₁₉ | 2456 |
| acaactgct | ATT ACA | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSGELVRHX₁₇X₁₈ | |
| ggg | ACT GCT | | TSGELVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆THLDLIRHX₁₇ | |
| | GGG | | GKSFSTHLDLIRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆SPADLTRH | |
| | | | KCPECGKSFSSPADLTRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆HKNALQ | |
| | | | GEKPYKCPECGKSFSHKNALQNH | | NHX₁₇X₁₈X₁₉HX₂₀X₂₁X₂₂X₂₃X₂₄X₈X₉CX₁₀X₁₁CX₁₂X₁₃X₁₄X₁₅X₁₆QNST | |
| | | | QRTHTGEKPYKCPECGKSFSQNS | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | TLTEHQRTHTGEKPYKCPECGKS | | DELVRHX₁₇X₁₈X₁₉HX₂₀X₂₁X₂₂X₂₃X₂₄X₂₅X₂₆X₂₇X₂₈X₂₉X₃₀ | |
| | | | FSRSDELVRHQRTHTGEKPTGKK | | | |
| | | | TS | | | |
| ggggtgcta | GGG gtg | 1359 | LEPGEKPYKCPECGKSFSTSGEL | 1908 | X₁X₂X₃X₄X₅X₆X₇X₈X₉CX₁₀X₁₁CX₁₂X₁₃X₁₄X₁₅X₁₆TSGELVRHX₁₇X₁₈X₁₉ | 2457 |
| attacaact | CTA ATT | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆THLDLIRHX₁₇X₁₈ | |
| gct | ACA ACT | | THLDLIRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆SPADLTRHX₁₇ | |
| | GCT | | GKSFSSPADLTRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆HKNALQNH | |
| | | | KCPECGKSFSHKNALQNHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QNS+32 | |
| | | | GEKPYKCPECGKSFSQNSTLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆RSDE | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | ELVRHQRTHTGEKPYKCPECGKS | | DKLVRHX₁₇X₁₃X₁₉HX₂₀X₂₁X₂₂X₂₃X₂₄X₂₅X₂₆X₂₇X₂₈X₂₉X₃₀ | |
| | | | FSRSDKLVRHQRTHTGEKPTGKK | | | |
| | | | TS | | | |
| actgctggg | ACT GCT | 1360 | LEPGEKPYKCPECGKSFSSKKAL | 1909 | X₁X₂X₃X₄X₅X₆X₇X₈X₉CX₁₀X₁₁CX₁₂X₁₃X₁₄X₁₅X₁₆SKKALTEHX₁₇X₁₈X₁₉ | 2458 |
| gccctaact | GGG GCC | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆THLDLIRHX₁₇X₁₈ | |
| cac | CTA ACT | | THLDLIRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QNSTLTEHX₁₇ | |
| | CAC | | GKSFSQNSTLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DCRDLARH | |
| | | | KCPECGKSFSDCRDLARHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDKLV | |
| | | | GEKPYKCPECGKSFSRSDKLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆TSGE | |
| | | | QRTHTGEKPYKCPECGKSFSTSG | | LVRHX₁₇X₁₈X₁₉HX₂₀X₂₁X₂₂X₂₃X₂₄X₈X₉CX₁₀X₁₁CX₁₂XEX₁₄X₁₅X₁₆TH | |
| | | | ELVRHQRTHTGEKPYKCPECGKS | | LDLIRHX₁₇X₁₃X₁₉HX₂₀X₂₁X₂₂X₂₃X₂₄X₂₅X₂₆X₂₇X₂₈X₂₉X₃₀ | |
| | | | FSTHLDLIRHQRTHTGEKPTGKK | | | |
| | | | TS | | | |
| cccggggtg | CCC GGG | 1361 | LEPGEKPYKCPECGKSFSTHLDL | 1910 | X₁X₂X₃X₄X₅X₆X₇X₈X₉CX₁₀X₁₁CX₁₂X₁₃X₁₄X₁₅X₁₆THLDLIRHX₁₇X₁₈X₁₉ | 2459 |
| ctaattaca | gtg CTA | | IRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆SPADLTRHX₁₇X₁₈ | |
| act | ATT ACA | | SPADLTRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆HKNALQNHX₁₇ | |
| | ACT | | GKSFSHKNALQNHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QNSTLTEH | |
| | | | KCPECGKSFSQNSTLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDELV | |
| | | | GEKPYKCPECGKSFSRSDELVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDK | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆SK | |
| | | | KLVRHQRTHTGEKPYKCPECGKS | | KHLAEHX₁₇X₁₈X₁₉HX₂₀X₂₁X₂₂X₂₃X₂₄X₂₅X₂₆X₂₇X₂₈X₂₉X₃₀ | |
| | | | FSSKKHLAEHQRTHTGEKPTGKK | | | |
| | | | TS | | | |
| tggctgaca | TGG CTG | 1362 | LEPGEKPYKCPECGKSFSQNSTL | 1911 | X₁X₂X₃X₄X₅X₆X₇X₈X₉CX₁₀X₁₁CX₁₂X₁₃X₁₄X₁₅X₁₆QNSTLTEHX₁₇X₁₈X₁₉ | 2460 |
| cccggggtg | ACA CCC | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDELVRHX₁₇X₁₈ | |
| cta | GGG gtg | | RSDELVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDKLVRHX₁₇ | |
| | CTA | | GKSFSRSDKLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆SKKHLAEH | |
| | | | KCPECGKSFSSKKHLAEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆SPADLT | |
| | | | GEKPYKCPECGKSFSSPADLTRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RNDA | |
| | | | QRTHTGEKPYKCPECGKSFSRND | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | ALTEHQRTHTGEKPYKCPECGKS | | DHLTTHX₁₇X₁₈X₁₉HX₂₀X₂₁X₂₂X₂₃X₂₄X₂₅X₂₆X₂₇X₂₈X₂₉X₃₀ | |
| | | | FSRSDHLTTHQRTHTGEKPTGKK | | | |
| | | | TS | | | |
| acacccggg | ACA CCC | 1363 | LEPGEKPYKCPECGKSFSSPADL | 1912 | X₁X₂X₃X₄X₅X₆X₇X₈X₉CX₁₀X₁₁CX₁₂X₁₃X₁₄X₁₅X₁₆SPADLTRHX₁₇X₁₈X₁₉ | 2461 |
| gtgctaatt | GGG gtg | | TRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆HKNALQNHX₁₇X₁₈ | |
| aca | CTA ATT | | HKNALQNHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QNSTLTEHX₁₇ | |
| | ACA | | GKSFSQNSTLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDELVRH | |
| | | | KCPECGKSFSRSDELVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDKLV | |
| | | | GEKPYKCPECGKSFSRSDKLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆SKKH | |
| | | | QRTHTGEKPYKCPECGKSFSSKK | | LAEHX₁₇X₁₈X₁₉HX₂₀X₂₁X₂₂X₂₃X₂₄X₈X₉CX₁₀X₁₁CX₁₂X₁₃X₁₄X₁₅X₁₆SP | |
| | | | HLAEHQRTHTGEKPYKCPECGKS | | ADLTRHX₁₇X₁₈X₁₉HX₂₀X₂₁X₂₂X₂₃X₂₄X₂₅X₂₆X₂₇X₂₈X₂₉X₃₀ | |
| | | | FSSPADLTRHQRTHTGEKPTGKK | | | |
| | | | TS | | | |
| ctgacaccc | CTG ACA | 1364 | LEPGEKPYKCPECGKSFSHKNAL | 1913 | X₁X₂X₃X₄X₅X₆X₇X₈X₉CX₁₀X₁₁CX₁₂X₁₃X₁₄X₁₅X₁₆HKNALQNHX₁₇X₁₈X₁₉ | 2462 |
| ggggtgcta | CCC GGG | | QNHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QNSTLTEHX₁₇X₁₈ | |
| att | gtg CTA | | QNSTLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDELVRHX₁₇ | |
| | ATT | | GKSFSRSDELVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDKLVRH | |
| | | | KCPECGKSFSRSDKLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆SKKHLA | |
| | | | GEKPYKCPECGKSFSSKKHLAEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆SPAD | |
| | | | QRTHTGEKPYKCPECGKSFSSPA | | LTRHX₁₇X₁₈X₁₉HX₂₀X₂₁X₂₂X₂₃X₂₄X₈X₉CX₁₀X₁₁CX₁₂XEX₁₄X₁₅X₁₆RN | |
| | | | DLTRHQRTHTGEKPYKCPECGKS | | DALTEHX₁₇X₁₈X₁₉HX₂₀X₂₁X₂₂X₂₃X₂₄X₂₅X₂₆X₂₇X₂₈X₂₉X₃₀ | |
| | | | FSRNDALTEHQRTHTGEKPTGKK | | | |
| | | | TS | | | |
| gctggggcc | GCT GGG | 1365 | LEPGEKPYKCPECGKSFSQSGHL | 1914 | X₁X₂X₃X₄X₅X₆X₇X₈X₉CX₁₀X₁₁CX₁₂X₁₃X₁₄X₁₅X₁₆QSGHLTEHX₁₇X₁₈X₁₉ | 2463 |
| ctaactcac | GCC CTA | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆SKKALTEHX₁₇X₁₈ | |
| cga | ACT CAC | | SKKALTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆THLDLIRHX₁₇ | |
| | CGA | | GKSFSTHLDLIRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QNSTLTEH | |
| | | | KCPECGKSFSQNSTLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DCRDLA | |
| | | | GEKPYKCPECGKSFSDCRDLARH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDK | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | KLVRHQRTHTGEKPYKCPECGKS | | GELVRHX₁₇X₁₈X₁₉HX₂₀X₂₁X₂₂X₂₃X₂₄X₂₅X₂₆X₂₇X₂₈X₂₉X₃₀ | |
| | | | FSTSGELVRHQRTHTGEKPTGKK | | | |
| | | | TS | | | |
| attgtacaa | ATT GTA | 1366 | LEPGEKPYKCPECGKSFSTSGNL | 1915 | X₁X₂X₃X₄X₅X₆X₇X₈X₉CX₁₀X₁₁CX₁₂X₁₃X₁₄X₁₅X₁₆TSGNLTEHX₁₇X₁₈X₁₉ | 2464 |
| ggcaggcat | CAA GGC | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSGNLTEHX₁₇X₁₈ | |
| cat | AGG CAT | | TSGNLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDHLTNHX₁₇ | |
| | CAT | | GKSFSRSDHLTNHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DPGHLVRH | |
| | | | KCPECGKSFSDPGHLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QSGNLT | |
| | | | GEKPYKCPECGKSFSQSGNLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆QSSS | |
| | | | QRTHTGEKPYKCPECGKSFSQSS | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆HK | |
| | | | SLVRHQRTHTGEKPYKCPECGKS | | NALQNHX₁₇X₁₃X₁₉HX₂₀X₂₁X₂₂X₂₃X₂₄X₂₅X₂₆X₂₇X₂₈X₂₉X₃₀ | |
| | | | FSHKNALQNHQRTHTGEKPTGKK | | | |
| | | | TS | | | |
| gtacaaggc | GTA CAA | 1367 | LEPGEKPYKCPECGKSFSDPGNL | 1916 | X₁X₂X₃X₄X₅X₆X₇X₈X₉CX₁₀X₁₁CX₁₂X₁₃X₁₄X₁₅X₁₆DPGNLVRHX₁₇X₁₈X₁₉ | 2465 |
| aggcatcat | GGC AGG | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSGNLTEHX₁₇X₁₈ | |
| gac | CAT CAT | | TSGNLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSGNLTEHX₁₇ | |
| | GAC | | GKSFSTSGNLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDHLTNH | |
| | | | KCPECGKSFSRSDHLTNHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DPGHLV | |
| | | | GEKPYKCPECGKSFSDPGHLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆QSGN | |
| | | | QRTHTGEKPYKCPECGKSFSQSG | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆QS | |
| | | | NLTEHQRTHTGEKPYKCPECGKS | | SSLVRHX₁₇X₁₃X₁₉HX₂₀X₂₁X₂₂X₂₃X₂₄X₂₅X₂₆X₂₇X₂₈X₂₉X₃₀ | |
| | | | FSQSSSLVRHQRTHTGEKPTGKK | | | |
| | | | TS | | | |
| atgtcaccc | ATT GTC | 1368 | LEPGEKPYKCPECGKSFSQSGDL | 1917 | X₁X₂X₃X₄X₅X₆X₇X₈X₉CX₁₀X₁₁CX₁₂X₁₃X₁₄X₁₅X₁₆QSGDLRRHX₁₇X₁₈X₁₉ | 2466 |
| ccaagtcag | ACC CCA | | RRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RADNLTEHX₁₇X₁₈ | |
| gca | AGT CAG | | RADNLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆HRTTLTNHX₁₇ | |
| | GCA | | GKSFSHRTTLTNHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSHSLTEH | |
| | | | KCPECGKSFSTSHSLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DKKDLT | |
| | | | GEKPYKCPECGKSFSDKKDLTRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆DPGA | |
| | | | QRTHTGEKPYKCPECGKSFSDPG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆HK | |
| | | | ALVRHQRTHTGEKPYKCPECGKS | | NALQNHX₁₇X₁₈X₁₉HX₂₀X₂₁X₂₂X₂₃X₂₄X₂₅X₂₆X₂₇X₂₈X₂₉X₃₀ | |
| | | | FSHKNALQNHQRTHTGEKPTGKK | | | |
| | | | TS | | | |
| gccattgtc | GCC ATT | 1369 | LEPGEKPYKCPECGKSFSRADNL | 1918 | X₁X₂X₃X₄X₅X₆X₇X₈X₉CX₁₀X₁₁CX₁₂X₁₃X₁₄X₁₅X₁₆RADNLTEHX₁₇X₁₈X₁₉ | 2467 |
| accccaagt | GTC ACC | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆HRTTLTNHX₁₇X₁₈ | |
| cag | CCA AGT | | HRTTLTNHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSHSLTEHX₁₇ | |
| | CAG | | GKSFSTSHSLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DKKDLTRH | |
| | | | KCPECGKSFSDKKDLTRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DPGALV | |
| | | | GEKPYKCPECGKSFSDPGALVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆HKNA | |
| | | | QRTHTGEKPYKCPECGKSFSHKN | | LQNHX₁₇X₁₈X₁₉HX₂₀X₂₁X₂₂X₂₃X₂₄X₈X₉CX₁₀X₁₁CX₁₂X₁₃X₁₄X₁₅X₁₆DC | |
| | | | ALQNHQRTHTGEKPYKCPECGKS | | RDLARHX₁₇X₁₈X₁₉HX₂₀X₂₁X₂₂X₂₃X₂₄X₂₅X₂₆X₂₇X₂₈X₂₉X₃₀ | |
| | | | FSDCRDLARHQRTHTGEKPTGKK | | | |
| | | | TS | | | |
| caagccatt | CAA GCC | 1370 | LEPGEKPYKCPECGKSFSHRTTL | 1919 | X₁X₂X₃X₄X₅X₆X₇X₈X₉CX₁₀X₁₁CX₁₂X₁₃X₁₄X₁₅X₁₆HRTTLTNHX₁₇X₁₈X₁₉ | 2468 |
| gtcacccca | ATT GTC | | TNHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSHSLTEHX₁₇X₁₈ | |
| agt | ACC CCA | | TSHSLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DKKDLTRHX₁₇ | |
| | AGT | | GKSFSDKKDLTRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DPGALVRH | |
| | | | KCPECGKSFSDPGALVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆HKNALQ | |
| | | | GEKPYKCPECGKSFSHKNALQNH | | NHX₁₇X₁₈X₁₉HX₂₀X₂₁X₂₂X₂₃X₂₄X₈X₉CX₁₀X₁₁CX₁₂X₁₃X₁₄X₁₅X₁₆DCRD | |
| | | | QRTHTGEKPYKCPECGKSFSDCR | | LARHX₁₇X₁₈X₁₉HX₂₀X₂₁X₂₂X₂₃X₂₄X₈X₉CX₁₀X₁₁CX₁₂X₁₃X₁₄X₁₅X₁₆QS | |
| | | | DLARHQRTHTGEKPYKCPECGKS | | GNLTEHX₁₇X₁₈X₁₉HX₂₀X₂₁X₂₂X₂₃X₂₄X₂₅X₂₆X₂₇X₂₈X₂₉X₃₀ | |
| | | | FSQSGNLTEHQRTHTGEKPTGKK | | | |
| | | | TS | | | |
| ggcactgac | GGC ACT | 1371 | LEPGEKPYKCPECGKSFSRNDTL | 1920 | X₁X₂X₃X₄X₅X₆X₇X₈X₉CX₁₀X₁₁CX₁₂X₁₃X₁₄X₁₅X₁₆RNDTLTEHX₁₇X₁₈X₁₉ | 2469 |
| agcctacct | GAC AGC | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TKNSLTEHX₁₇X₁₈ | |
| ccg | CTA CCT | | TKNSLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QNSTLTEHX₁₇ | |
| | CCG | | GKSFSQNSTLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆ERSHLREH | |
| | | | KCPECGKSFSERSHLREHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DPGNLV | |
| | | | GEKPYKCPECGKSFSDPGNLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆THLD | |
| | | | QRTHTGEKPYKCPECGKSFSTHL | | LIRHX₁₇X₁₈X₁₉HX₂₀X₂₁X₂₂X₂₃X₂₄X₈X₉CK₁₀X₁₁CX₁₂X₁₃X₁₄X₁₅X₁₆DP | |
| | | | DLIRHQRTHTGEKPYKCPECGKS | | GHLVRHX₁₇X₁₈X₁₉HX₂₀X₂₁X₂₂X₂₃X₂₄X₂₅X₂₆X₂₇X₂₈X₂₉X₃₀ | |
| | | | FSDPGHLVRHQRTHTGEKPTGKK | | | |
| | | | TS | | | |
| actgacagc | ACT GAC | 1372 | LEPGEKPYKCPECGKSFSQLAHL | 1921 | X₁X₂X₃X₄X₅X₆X₇X₈X₉CX₁₀X₁₁CX₁₂X₁₃X₁₄X₁₅X₁₆QLAHLRAHX₁₇X₁₈X₁₉ | 2470 |
| ctacctccg | AGC CTA | | RAHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RNDTLTEHX₁₇X₁₈ | |
| aga | CCT CCG | | RNDTLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TKNSLTEHX₁₇ | |
| | AGA | | GKSFSTKNSLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QNSTLTEH | |
| | | | KCPECGKSFSQNSTLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆ERSHLR | |
| | | | GEKPYKCPECGKSFSERSHLREH | | EHX₁₇X₁₈X₁₉HX₂₀X₂₁X₂₂X₂₃X₂₄X₈X₉CX₁₀X₁₁CX₁₂X₁₃X₁₄X₁₅X₁₆DPGN | |
| | | | QRTHTGEKPYKCPECGKSFSDPG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆TH | |
| | | | NLVRHQRTHTGEKPYKCPECGKS | | LDLIRHX₁₇X₁₈X₁₉HX₂₀X₂₁X₂₂X₂₃X₂₄X₂₅X₂₆X₂₇X₂₈X₂₉X₃₀ | |
| | | | FSTHLDLIRHQRTHTGEKPTGKK | | | |
| | | | TS | | | |
| ccaaatcat | CCA AAT | 1373 | LEPGEKPYKCPECGKSFSSKKHL | 1922 | X₁X₂X₃X₄X₅X₆X₇X₈X₉CX₁₀X₁₁CX₁₂X₁₃X₁₄X₁₅X₁₆SKKHLAEHX₁₇X₁₈X₁₉ | 2471 |
| tgacttcta | CAT TGA | | AEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QNSTLTEHX₁₇X₁₈ | |
| ccc | CTT CTA | | QNSTLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TTGALTEHX₁₇ | |
| | CCC | | GKSFSTTGALTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QAGHLASH | |
| | | | KCPECGKSFSQAGHLASHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSGNLT | |
| | | | GEKPYKCPECGKSFSTSGNLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆TTGN | |
| | | | QRTHTGEKPYKCPECGKSFSTTG | | LTVHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | NLTVHQRTHTGEKPYKCPECGKS | | HSLTEHX₁₇X₁₈X₁₉HX₂₀X₂₁X₂₂X₂₃X₂₄X₂₅X₂₆X₂₇X₂₈X₂₉X₃₀ | |
| | | | FSTSHSLTEHQRTHTGEKPTGKK | | | |
| | | | TS | | | |
| gcagagata | GCA gag | 1374 | LEPGEKPYKCPECGKSFSTSGNL | 1923 | X₁X₂X₃X₄X₅X₆X₇X₈X₉CX₁₀X₁₁CX₁₂X₁₃X₁₄X₁₅X₁₆TSGNLTEHX₁₇X₁₈X₁₉ | 2472 |
| aggctgccc | ATA AGG | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆SKKHLAEHX₁₇X₁₈ | |
| cat | CTG CCC | | SKKHLAEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RNDALTEHX₁₇ | |
| | CAT | | GKSFSRNDALTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDHLTNH | |
| | | | KCPECGKSFSRSDHLTNHQRTHT | | X₁₇X₁₈X₁₉HX₂₀X₂₁X₂₂X₂₃X₂₄X₈X₉CXHX₁₁CX₁₂X₁₃X₁₄X₁₅X₁₆QKSSLI | |
| | | | GEKPYKCPECGKSFSQKSSLIAH | | AHX₁₇X₁₈X₁₉HX₂₀X₂₁X₂₂X₂₃X₂₄X₈X₉CX₁₀X₁₁CX₁₂X₁₃X₁₄X₁₅X₁₆RSDN | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆QS | |
| | | | NLVRHQRTHTGEKPYKCPECGKS | | GDLRRHX₁₇X₁₈X₁₉HX₂₀X₂₁X₂₂X₂₃X₂₄X₂₅X₂₆X₂₇X₂₈X₂₉X₃₀ | |
| | | | FSQSGDLRRHQRTHTGEKPTGKK | | | |
| | | | TS | | | |
| gagataagg | gag ATA | 1375 | LEPGEKPYKCPECGKSFSDPGHL | 1924 | X₁X₂X₃X₄X₅X₆X₇X₈X₉CX₁₀X₁₁CX₁₂X₁₃X₁₄X₁₅X₁₆DPGHLVRHX₁₇X₁₈X₁₉ | 2473 |
| ctgccccat | AGG CTG | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSGNLTEHX₁₇X₁₈ | |
| ggc | CCC CAT | | TSGNLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆SKKHLAEHX₁₇ | |
| | GGC | | GKSFSSKKHLAEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RNDALTEH | |
| | | | KCPECGKSFSRNDALTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDHLT | |
| | | | GEKPYKCPECGKSFSRSDHLTNH | | NHX₁₇X₁₈X₁₉HX₂₀X₂₁X₂₂X₂₃X₂₄X₈X₉CX₁₀X₁₁CX₁₂X₁₃X₁₄X₁₅X₁₆QKSS | |
| | | | QRTHTGEKPYKCPECGKSFSQKS | | LIAHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | SLIAHQRTHTGEKPYKCPECGKS | | DNLVRHX₁₇X₁₈X₁₉HX₂₀X₂₁X₂₂X₂₃X₂₄X₂₅X₂₆X₂₇X₂₈X₂₉X₃₀ | |
| | | | FSRSDNLVRHQRTHTGEKPTGKK | | | |
| | | | TS | | | |
| ataaggctg | ATA AGG | 1376 | LEPGEKPYKCPECGKSFSTSHSL | 1925 | X₁X₂X₃X₄X₅X₆X₇X₈X₉CX₁₀X₁₁CX₁₂X₁₃X₁₄X₁₅X₁₆TSHSLTEHX₁₇X₁₈X₁₉ | 2474 |
| ccccatggc | CTG CCC | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆DPGHLVRHX₁₇X₁₈ | |
| cca | CAT GGC | | DPGHLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSGNLTEHX₁₇ | |
| | CCA | | GKSFSTSGNLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆SKKHLAEH | |
| | | | KCPECGKSFSSKKHLAEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RNDALT | |
| | | | GEKPYKCPECGKSFSRNDALTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆RSDH | |
| | | | QRTHTGEKPYKCPECGKSFSRSD | | LTNHX₁₇X₁₈X₁₉HX₂₀X₂₁X₂₂X₂₃X₂₄X₈X₉CX₁₀X₁₁CX₁₂X₁₃X₁₄X₁₅X₁₆QK | |
| | | | HLTNHQRTHTGEKPYKCPECGKS | | SSLIAHX₁₇X₁₈X₁₉HX₂₀X₂₁X₂₂X₂₃X₂₄X₂₅X₂₆X₂₇X₂₈X₂₉X₃₀ | |
| | | | FSQKSSLIAHQRTHTGEKPTGKK | | | |
| | | | TS | | | |
| aggctgccc | AGG CTG | 1377 | LEPGEKPYKCPECGKSFSQSGHL | 1926 | X₁X₂X₃X₄X₅X₆X₇X₈X₉CX₁₀X₁₁CX₁₂X₁₃X₁₄X₁₅X₁₆QSGHLTEHX₁₇X₁₈X₁₉ | 2475 |
| catggccca | CCC CAT | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSHSLTEHX₁₇X₁₈ | |
| cga | GGC CCA | | TSHSLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DPGHLVRHX₁₇ | |
| | CGA | | GKSFSDPGHLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSGNLTEH | |
| | | | KCPECGKSFSTSGNLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆SKKHLA | |
| | | | GEKPYKCPECGKSFSSKKHLAEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆RNDA | |
| | | | QRTHTGEKPYKCPECGKSFSRND | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | ALTEHQRTHTGEKPYKCPECGKS | | DHLTNHX₁₇X₁₈X₁₉HX₂₀X₂₁X₂₂X₂₃X₂₄X₂₅X₂₆X₂₇X₂₈X₂₉X₃₀ | |
| | | | FSRSDHLTNHQRTHTGEKPTGKK | | | |
| | | | TS | | | |
| agagataag | AGA GAT | 1378 | LEPGEKPYKCPECGKSFSRSDHL | 1927 | X₁X₂X₃X₄X₅X₆X₇X₈X₉CX₁₀X₁₁CX₁₂X₁₃X₁₄X₁₅X₁₆RSDHLTTHX₁₇X₁₈X₁₉ | 2476 |
| gctgcccca | AAG GCT | | TTHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSHSLTEHX₁₇X₁₈ | |
| tgg | GCC CCA | | TSHSLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DCRDLARHX₁₇ | |
| | TGG | | GKSFSDCRDLARHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSGELVRH | |
| | | | KCPECGKSFSTSGELVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RKDNLK | |
| | | | GEKPYKCPECGKSFSRKDNLKNH | | NHX₁₇X₁₈X₁₉HX₂₀X₂₁X₂₂X₂₃X₂₄X₈X₉CX₁₀X₁₁CX₁₂X₁₃X₁₄X₁₅X₁₆TSGN | |
| | | | QRTHTGEKPYKCPECGKSFSTSG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆QL | |
| | | | NLVRHQRTHTGEKPYKCPECGKS | | AHLRAHX₁₇X₁₈X₁₉HX₂₀X₂₁X₂₂X₂₃X₂₄X₂₅X₂₆X₂₇X₂₈X₂₉X₃₀ | |
| | | | FSQLAHLRAHQRTHTGEKPTGKK | | | |
| | | | TS | | | |
| cccacgatt | CCC ACG | 1379 | LEPGEKPYKCPECGKSFSQRANL | 1928 | X₁X₂X₃X₄X₅X₆X₇X₈X₉CX₁₀X₁₁CX₁₂X₁₃X₁₄X₁₅X₁₆QRANLRAHX₁₇X₁₈X₁₉ | 2477 |
| tagaaacct | ATT TAG | | RAHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TKNSLTEHX₁₇X₁₈ | |
| aaa | AAA CCT | | TKNSLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QRANLRAHX₁₇ | |
| | AAA | | GKSFSQRANLRAHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆REDNLHTH | |
| | | | KCPECGKSFSREDNLHTHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆HKNALQ | |
| | | | GEKPYKCPECGKSFSHKNALQNH | | NHX₁₇X₁₈X₁₉HX₂₀X₂₁X₂₂X₂₃X₂₄X₈X₉CX₁₀X₁₁CX₁₂X₁₃X₁₄X₁₅X₁₆RTDT | |
| | | | QRTHTGEKPYKCPECGKSFSRTD | | LRDHX₁₇X₁₈X₁₉HX₂₀X₂₁X₂₂X₂₃X₂₄X₈X₉CX₁₀X₁₁CX₁₂X₁₃X₁₄X₁₅X₁₆SK | |
| | | | TLRDHQRTHTGEKPYKCPECGKS | | KHLAEHX₁₇X₁₈X₁₉HX₂₀X₂₁X₂₂X₂₃X₂₄X₂₅X₂₆X₂₇X₂₈X₂₉X₃₀ | |
| | | | FSSKKHLAEHQRTHTGEKPTGKK | | | |
| | | | TS | | | |
| tggcccacg | TGG CCC | 1380 | LEPGEKPYKCPECGKSFSTKNSL | 1929 | X₁X₂X₃X₄X₅X₆X₇X₈X₉CX₁₀X₁₁CX₁₂X₁₃X₁₄X₁₅X₁₆TKNSLTEHX₁₇X₁₈X₁₉ | 2478 |
| atttagaaa | ACG ATT | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QRANLRAHX₁₇X₁₈ | |
| cct | TAG AAA | | QRANLRAHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆REDNLHTHX₁₇ | |
| | CCT | | GKSFSREDNLHTHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆HKNALQNH | |
| | | | KCPECGKSFSHKNALQNHQRTHT | | X₁₇X₁₈X₁₉HX₂₀X₂₁X₂₂X₂₃X₂₄X₈X₉CXHX₁₁CX₁₂X₁₃X₁₄X₁₅X₁₆RTDTLR | |
| | | | GEKPYKCPECGKSFSRTDTLRDH | | DHX₁₇X₁₈X₁₉HX₂₀X₂₁X₂₂X₂₃X₂₄X₈X₉CX₁₀X₁₁CX₁₂X₁₃X₁₄X₁₅X₁₆SKKH | |
| | | | QRTHTGEKPYKCPECGKSFSSKK | | LAEHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | HLAEHQRTHTGEKPYKCPECGKS | | DHLTTHX₁₇X₁₈X₁₉HX₂₀X₂₁X₂₂X₂₃X₂₄X₂₅X₂₆X₂₇X₂₈X₂₉X₃₀ | |
| | | | FSRSDHLTTHQRTHTGEKPTGKK | | | |
| | | | TS | | | |
| ccatggccc | CCA TGG | 1381 | LEPGEKPYKCPECGKSFSQRANL | 1930 | X₁X₂X₃X₄X₅X₆X₇X₈X₉CX₁₀X₁₁CX₁₂X₁₃X₁₄X₁₅X₁₆QRANLRAHX₁₇X₁₈X₁₉ | 2479 |
| acgatttag | CCC ACG | | RAHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆REDNLHTHX₁₇X₁₈ | |
| aaa | ATT TAG | | REDNLHTHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆HKNALQNHX₁₇ | |
| | AAA | | GKSFSHKNALQNHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RTDTLRDH | |
| | | | KCPECGKSFSRTDTLRDHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆SKKHLA | |
| | | | GEKPYKCPECGKSFSSKKHLAEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆RSDH | |
| | | | QRTHTGEKPYKCPECGKSFSRSD | | LTTHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | HLTTHQRTHTGEKPYKCPECGKS | | HSLTEHX₁₇X₁₈X₁₉HX₂₀X₂₁X₂₂X₂₃X₂₄X₂₅X₂₆X₂₇X₂₈X₂₉X₃₀ | |
| | | | FSTSHSLTEHQRTHTGEKPTGKK | | | |
| | | | TS | | | |
| gccccatgg | GCC CCA | 1382 | LEPGEKPYKCPECGKSFSREDNL | 1931 | X₁X₂X₃X₄X₅X₆X₇X₈X₉CX₁₀X₁₁CX₁₂X₁₃X₁₄X₁₅X₁₆REDNLHTHX₁₇X₁₈X₁₉ | 2480 |
| cccacgatt | TGG CCC | | HTHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆HKNALQNHX₁₇X₁₈ | |
| tag | ACG ATT | | HKNALQNHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RTDTLRDHX₁₇ | |
| | TAG | | GKSFSRTDTLRDHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆SKKHLAEH | |
| | | | KCPECGKSFSSKKHLAEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDHLT | |
| | | | GEKPYKCPECGKSFSRSDHLTTH | | THX₁₇X₁₈X₁₉HX₂₀X₂₁X₂₂X₂₃X₂₄X₈X₉CX₁₀X₁₁CX₁₂X₁₃X₁₄X₁₅X₁₆TSHS | |
| | | | QRTHTGEKPYKCPECGKSFSTSH | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆DC | |
| | | | SLTEHQRTHTGEKPYKCPECGKS | | RDLARHX₁₇X₁₈X₁₉HX₂₀X₂₁X₂₂X₂₃X₂₄X₂₅X₂₆X₂₇X₂₈X₂₉X₃₀ | |
| | | | FSDCRDLARHQRTHTGEKPTGKK | | | |
| | | | TS | | | |
| gataaggct | GAT AAG | 1383 | LEPGEKPYKCPECGKSFSSKKHL | 1932 | X₁X₂X₃X₄X₅X₆X₇X₈X₉CX₁₀X₁₁CX₁₂X₁₃X₁₄X₁₅X₁₆SKKHLAEHX₁₇X₁₈X₁₉ | 2481 |
| gccccatgg | GCT GCC | | AEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDHLTTHX₁₇X₁₈ | |
| ccc | CCA TGG | | RSDHLTTHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSHSLTEHX₁₇ | |
| | CCC | | GKSFSTSHSLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DCRDLARH | |
| | | | KCPECGKSFSDCRDLARHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSGELV | |
| | | | GEKPYKCPECGKSFSTSGELVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RKDN | |
| | | | QRTHTGEKPYKCPECGKSFSRKD | | LKNHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | NLKNHQRTHTGEKPYKCPECGKS | | GNLVRHX₁₇X₁₈X₁₉HX₂₀X₂₁X₂₂X₂₃X₂₄X₂₅X₂₆X₂₇X₂₈X₂₉X₃₀ | |
| | | | FSTSGNLVRHQRTHTGEKPTGKK | | | |
| | | | TS | | | |
| gctgcccca | GCT GCC | 1384 | LEPGEKPYKCPECGKSFSHKNAL | 1933 | X₁X₂X₃X₄X₅X₆X₇X₈X₉CX₁₀X₁₁CX₁₂X₁₃X₁₄X₁₅X₁₆HKNALQNHX₁₇X₁₈X₁₉ | 2482 |
| tggcccacg | CCA TGG | | QNHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RTDTLRDHX₁₇X₁₈ | |
| att | CCC ACG | | RTDTLRDHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆SKKHLAEHX₁₇ | |
| | ATT | | GKSFSSKKHLAEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDHLTTH | |
| | | | KCPECGKSFSRSDHLTTHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSHSLT | |
| | | | GEKPYKCPECGKSFSTSHSLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆DCRD | |
| | | | QRTHTGEKPYKCPECGKSFSDCR | | LARHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | DLARHQRTHTGEKPYKCPECGKS | | GELVRHX₁₇X₁₈X₁₉HX₂₀X₂₁X₂₂X₂₃X₂₄X₂₅X₂₆X₂₇X₂₈X₂₉X₃₀ | |
| | | | FSTSGELVRHQRTHTGEKPTGKK | | | |
| | | | TS | | | |
| aaggctgcc | AAG GCT | 1385 | LEPGEKPYKCPECGKSFSRTDTL | 1934 | X₁X₂X₃X₄X₅X₆X₇X₈X₉CX₁₀X₁₁CX₁₂X₁₃X₁₄X₁₅X₁₆RTDTLRDHX₁₇X₁₈X₁₉ | 2483 |
| ccatggccc | GCC CCA | | RDHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆SKKHLAEHX₁₇X₁₈ | |
| acg | TGG CCC | | SKKHLAEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDHLTTHX₁₇ | |
| | ACG | | GKSFSRSDHLTTHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSHSLTEH | |
| | | | KCPECGKSFSTSHSLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DCRDLA | |
| | | | GEKPYKCPECGKSFSDCRDLARH | | RHX₁₇X₁₈X₁₉HX₂₀X₂₁X₂₂X₂₃X₂₄X₈X₉CX₁₀X₁₁CX₁₂X₁₃X₁₄X₁₅X₁₆TSGE | |
| | | | QRTHTGEKPYKCPECGKSFSTSG | | LVRHX₁₇X₁₈X₁₉HX₂₀X₂₁X₂₂X₂₃X₂₄X₈X₉CX₁₀X₁₁CX₁₂XEX₁₄X₁₅X₁₆RK | |
| | | | ELVRHQRTHTGEKPYKCPECGKS | | DNLKNHX₁₇X₁₈X₁₉HX₂₀X₂₁X₂₂X₂₃X₂₄X₂₅X₂₆X₂₇X₂₈X₂₉X₃₀ | |
| | | | FSRKDNLKNHQRTHTGEKPTGKK | | | |
| | | | TS | | | |
| agaaaccta | AGA AAC | 1386 | LEPGEKPYKCPECGKSFSSKKHL | 1935 | X₁X₂X₃X₄X₅X₆X₇X₈X₉CX₁₀X₁₁CX₁₂X₁₃X₁₄X₁₅X₁₆SKKHLAEHX₁₇X₁₈X₁₉ | 2484 |
| aatcccagg | CTA AAT | | AEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDHLTNHX₁₇X₁₈ | |
| ccc | CCC AGG | | RSDHLTNHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆SKKHLAEHX₁₇ | |
| | CCC | | GKSFSSKKHLAEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TTGNLTVH | |
| | | | KCPECGKSFSTTGNLTVHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QNSTLT | |
| | | | GEKPYKCPECGKSFSQNSTLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆DSGN | |
| | | | QRTHTGEKPYKCPECGKSFSDSG | | LRVHX₁₇X₁₈X₁₉HX₂₀X₂₁X₂₂X₂₃X₂₄X₈X₉CX₁₀X₁₁CX₁₂X₁₃X₁₄X₁₅X₁₆QL | |
| | | | NLRVHQRTHTGEKPYKCPECGKS | | AHLRAHX₁₇X₁₈X₁₉HX₂₀X₂₁X₂₂X₂₃X₂₄X₂₅X₂₆X₂₇X₂₈X₂₉X₃₀ | |
| | | | FSQLAHLRAHQRTHTGEKPTGKK | | | |
| | | | TS | | | |
| aacctaaat | AAC CTA | 1387 | LEPGEKPYKCPECGKSFSRADNL | 1936 | X₁X₂X₃X₄X₅X₆X₇X₈X₉CX₁₀X₁₁CX₁₂X₁₃X₁₄X₁₅X₁₆RADNLTEHX₁₇X₁₈X₁₉ | 2485 |
| cccaggccc | AAT CCC | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆SKKHLAEHX₁₇X₁₈ | |
| cag | AGG CCC | | SKKHLAEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDHLTNHX₁₇ | |
| | CAG | | GKSFSRSDHLTNHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆SKKHLAEH | |
| | | | KCPECGKSFSSKKHLAEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TTGNLT | |
| | | | GEKPYKCPECGKSFSTTGNLTVH | | VHX₁₇X₁₈X₁₉HX₂₀X₂₁X₂₂X₂₃X₂₄X₈X₉CX₁₀X₁₁CX₁₂X₁₃X₁₄X₁₅X₁₆QNST | |
| | | | QRTHTGEKPYKCPECGKSFSQNS | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆DS | |
| | | | TLTEHQRTHTGEKPYKCPECGKS | | GNLRVHX₁₇X₁₃X₁₉HX₂₀X₂₁X₂₂X₂₃X₂₄X₂₅X₂₆X₂₇X₂₈X₂₉X₃₀ | |
| | | | FSDSGNLRVHQRTHTGEKPTGKK | | | |
| | | | TS | | | |
| ctaaatccc | CTA AAT | 1388 | LEPGEKPYKCPECGKSFSRRDEL | 1937 | X₁X₂X₃X₄X₅X₆X₇X₈X₉CX₁₀X₁₁CX₁₂X₁₃X₁₄X₁₅X₁₆RRDELNVHX₁₇X₁₈X₁₉ | 2486 |
| aggccccag | CCC AGG | | NVHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RADNLTEHX₁₇X₁₈ | |
| atg | CCC CAG | | RADNLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆SKKHLAEHX₁₇ | |
| | ATG | | GKSFSSKKHLAEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDHLTNH | |
| | | | KCPECGKSFSRSDHLTNHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆SKKHLA | |
| | | | GEKPYKCPECGKSFSSKKHLAEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆TTGN | |
| | | | QRTHTGEKPYKCPECGKSFSTTG | | LTVHX₁₇X₁₈X₁₉HX₂₀X₂₁X₂₂X₂₃X₂₄X₈X₉CX₁₀X₁₁CX₁₂XEX₁₄X₁₅X₁₆QN | |
| | | | NLTVHQRTHTGEKPYKCPECGKS | | STLTEHX₁₇X₁₃X₁₉HX₂₀X₂₁X₂₂X₂₃X₂₄X₂₅X₂₆X₂₇X₂₈X₂₉X₃₀ | |
| | | | FSQNSTLTEHQRTHTGEKPTGKK | | | |
| | | | TS | | | |
| aatcccagg | AAT CCC | 1389 | LEPGEKPYKCPECGKSFSTSHSL | 1938 | X₁X₂X₃X₄X₅X₆X₇X₈X₉CX₁₀X₁₁CX₁₂X₁₃X₁₄X₁₅X₁₆TSHSLTEHX₁₇X₁₈X₁₉ | 2487 |
| ccccagatg | AGG CCC | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RRDELNVHX₁₇X₁₈ | |
| cca | CAG ATG | | RRDELNVHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RADNLTEHX₁₇ | |
| | CCA | | GKSFSRADNLTEHQRTHTGEKPY | | X₁₇X₁₈X₁₉HX₂₀X₂₁X₂₂X₂₃X₂₄X₈X₉CX₁₀X₁₁CX₁₂X₁₃X₁₄X₁₅X₁₆SKKHLAEH | |
| | | | KCPECGKSFSSKKHLAEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDHLT | |
| | | | GEKPYKCPECGKSFSRSDHLTNH | | NHX₁₇X₁₈X₁₉HX₂₀X₂₁X₂₂X₂₃X₂₄X₈X₉CX₁₀X₁₁CX₁₂X₁₃X₁₄X₁₅X₁₆SKKH | |
| | | | QRTHTGEKPYKCPECGKSFSSKK | | LAEHX₁₇X₁₈X₁₉HX₂₀X₂₁X₂₂X₂₃X₂₄X₈X₉CX₁₀X₁₁CX₁₂X₁₃X₁₄X₁₅X₁₆TT | |
| | | | HLAEHQRTHTGEKPYKCPECGKS | | GNLTVHX₁₇X₁₈X₁₉HX₂₀X₂₁X₂₂X₂₃X₂₄X₂₅X₂₆X₂₇X₂₈X₂₉X₃₀ | |
| | | | FSTTGNLTVHQRTHTGEKPTGKK | | | |
| | | | TS | | | |
| caggcccca | CAG GCC | 1390 | LEPGEKPYKCPECGKSFSTTGAL | 1939 | X₁X₂X₃X₄X₅X₆X₇X₈X₉CX₁₀X₁₁CX₁₂X₁₃X₁₄X₁₅X₁₆TTGALTEHX₁₇X₁₈X₁₉ | 2488 |
| gatgccaat | CCA GAT | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TTGNLTVHX₁₇X₁₈ | |
| ctt | GCC AAT | | TTGNLTVHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DCRDLARHX₁₇ | |
| | CTT | | GKSFSDCRDLARHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSGNLVRH | |
| | | | KCPECGKSFSTSGNLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSHSLT | |
| | | | GEKPYKCPECGKSFSTSHSLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆DCRD | |
| | | | QRTHTGEKPYKCPECGKSFSDCR | | LARHX₁₇X₁₈X₁₉HX₂₀X₂₁X₂₂X₂₃X₂₄X₈X₉CX₁₀X₁₁CX₁₂X₁₃X₁₄X₁₅X₁₆RA | |
| | | | DLARHQRTHTGEKPYKCPECGKS | | DNLTEHX₁₇X₁₈X₁₉HX₂₀X₂₁X₂₂X₂₃X₂₄X₂₅X₂₆X₂₇X₂₈X₂₉X₃₀ | |
| | | | FSRADNLTEHQRTHTGEKPTGKK | | | |
| | | | TS | | | |
| gatgccaat | GAT GCC | 1391 | LEPGEKPYKCPECGKSFSTKNSL | 1940 | X₁X₂X₃X₄X₅X₆X₇X₈X₉CX₁₀X₁₁CX₁₂X₁₃X₁₄X₁₅X₁₆TKNSLTEHX₁₇X₁₈X₁₉ | 2489 |
| cttctggat | AAT CTT | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSGNLVRHX₁₇X₁₈ | |
| cct | CTG GAT | | TSGNLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RNDALTEHX₁₇ | |
| | CCT | | GKSFSRNDALTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TTGALTEH | |
| | | | KCPECGKSFSTTGALTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TTGNLT | |
| | | | GEKPYKCPECGKSFSTTGNLTVH | | VHX₁₇X₁₈X₁₉HX₂₀X₂₁X₂₂X₂₃X₂₄X₈X₉CX₁₀X₁₁CX₁₂X₁₃X₁₄X₁₅X₁₆DCRD | |
| | | | QRTHTGEKPYKCPECGKSFSDCR | | LARHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | DLARHQRTHTGEKPYKCPECGKS | | GNLVRHX₁₇X₁₈X₁₉HX₂₀X₂₁X₂₂X₂₃X₂₄X₂₅X₂₆X₂₇X₂₈X₂₉X₃₀ | |
| | | | FSTSGNLVRHQRTHTGEKPTGKK | | | |
| | | | TS | | | |
| gccccagat | GCC CCA | 1392 | LEPGEKPYKCPECGKSFSRNDAL | 1941 | X₁X₂X₃X₄X₅X₆X₇X₈X₉CX₁₀X₁₁CX₁₂X₁₃X₁₄X₁₅X₁₆RNDALTEHX₁₇X₁₈X₁₉ | 2490 |
| gccaatctt | GAT GCC | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TTGALTEHX₁₇X₁₈ | |
| ctg | AAT CTT | | TTGALTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TTGNLTVHX₁₇ | |
| | CTG | | GKSFSTTGNLTVHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆DCRDLARH | |
| | | | KCPECGKSFSDCRDLARHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSGNLV | |
| | | | GEKPYKCPECGKSFSTSGNLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆TSHS | |
| | | | QRTHTGEKPYKCPECGKSFSTSH | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆DC | |
| | | | SLTEHQRTHTGEKPYKCPECGKS | | RDLARHX₁₇X₁₈X₁₉HX₂₀X₂₁X₂₂X₂₃X₂₄X₂₅X₂₆X₂₇X₂₈X₂₉X₃₀ | |
| | | | FSDCRDLARHQRTHTGEKPTGKK | | | |
| | | | TS | | | |
| ccagatgcc | CCA GAT | 1393 | LEPGEKPYKCPECGKSFSTSGNL | 1942 | X₁X₂X₃X₄X₅X₆X₇X₈X₉CX₁₀X₁₁CX₁₂X₁₃X₁₄X₁₅X₁₆TSGNLVRHX₁₇X₁₈X₁₉ | 2491 |
| aatcttctg | GCC AAT | | VRHQRTHTGEKPYKCPECGKSFS | | HX₂₀X₂₁X₂₂X₂₃X₂₄X₈X₉CX₁₀X₁₁CX₁₂XEX₁₄X₁₅X₁₆RNDALTEHX₁₇X₁₈ | |
| gat | CTT CTG | | RNDALTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TTGALTEHX₁₇ | |
| | GAT | | GKSFTTGALTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TTGNLTVH | |
| | | | KCPECGKSFSTTGNLTVHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆DCRDLA | |
| | | | GEKPYKCPECGKSFSDCRDLARH | | RHX₁₇X₁₈X₁₉HX₂₀X₂₁X₂₂X₂₃X₂₄X₈X₉CX₁₀X₁₁CX₁₂X₁₃X₁₄X₁₅X₁₆TSGN | |
| | | | QRTHTGEKPYKCPECGKSFSTSG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | NLVRHQRTHTGEKPYKCPECGKS | | HSLTEHX₁₇X₁₈X₁₉HX₂₀X₂₁X₂₂X₂₃X₂₄X₂₅X₂₆X₂₇X₂₈X₂₉X₃₀ | |
| | | | FSTSHSLTEHQRTHTGEKPTGKK | | | |
| | | | TS | | | |
| ctgggagca | CTG GGA | 1394 | LEPGEKPYKCPECGKSFSQRAHL | 1943 | X₁X₂X₃X₄X₅X₆X₇X₈X₉CX₁₀X₁₁CX₁₂X₁₃X₁₄X₁₅X₁₆QRAHLERHX₁₇X₁₈X₁₉ | 2492 |
| gaatggact | GCA GAA | | ERHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆THLDLIRHX₁₇X₁₈ | |
| gga | TGG ACT | | THLDLIRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDHLTTHX₁₇ | |
| | GGA | | GKSFSRSDHLTTHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QSSNLVRH | |
| | | | KCPECGKSFSQSSNLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QSGDLR | |
| | | | GEKPYKCPECGKSFSQSGDLRRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆QRAH | |
| | | | QRTHTGEKPYKCPECGKSFSQRA | | LERHX₁₇X₁₈X₁₉HX₂₀X₂₁X₂₂X₂₃X₂₄X₈X₉CX₁₀X₁₁CX₁₂X₁₃X₁₄X₁₅X₁₆RN | |
| | | | HLERHQRTHTGEKPYKCPECGKS | | DALTEHX₁₇X₁₃X₁₉HX₂₀X₂₁X₂₂X₂₃X₂₄X₂₅X₂₆X₂₇X₂₈X₂₉X₃₀ | |
| | | | FSRNDALTEHQRTHTGEKPTGKK | | | |
| | | | TS | | | |
| cccctggga | CCC CTG | 1395 | LEPGEKPYKCPECGKSFSTHLDL | 1944 | X₁X₂X₃X₄X₅X₆X₇X₈X₉CX₁₀X₁₁CX₁₂X₁₃X₁₄X₁₅X₁₆THLDLIRHX₁₇X₁₈X₁₉ | 2493 |
| gcagaatgg | GGA GCA | | IRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDHLTTHX₁₇X₁₈ | |
| act | GAA TGG | | RSDHLTTHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QSSNLVRHX₁₁ | |
| | ACT | | GKSFSQSSNLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QSGDLRRH | |
| | | | KCPECGKSFSQSGDLRRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QRAHLE | |
| | | | GEKPYKCPECGKSFSQRAHLERH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RNDA | |
| | | | QRTHTGEKPYKCPECGKSFSRND | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆SK | |
| | | | ALTEHQRTHTGEKPYKCPECGKS | | KHLAEHX₁₇X₁₃X₁₉HX₂₀X₂₁X₂₂X₂₃X₂₄X₂₅X₂₆X₂₇X₂₈X₂₉X₃₀ | |
| | | | FSSKKHLAEHQRTHTGEKPTGKK | | | |
| | | | TS | | | |
| ggagcagaa | GGA GCA | 1396 | LEPGEKPYKCPECGKSFSHRTTL | 1945 | X₁X₂X₃X₄X₅X₆X₇X₈X₉CX₁₀X₁₁CX₁₂X₁₃X₁₄X₁₅X₁₆HRTTLTNHX₁₇X₁₈X₁₉ | 2494 |
| tggactgga | GAA TGG | | TNHQRTHTGEKPYKCPECGKSFS | | HX₂₀X₂₁X₂₂X₂₃X₂₄X₈X₉CX₁₀X₁₁CX₁₂XEX₁₄X₁₅X₁₆QRAHLERHX₁₇X₁₈ | |
| agt | ACT GGA | | QRAHLERHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆THLDLIRHX₁₇ | |
| | AGT | | GKSFSTHLDLIRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDHLTTH | |
| | | | KCPECGKSFSRSDHLTTHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QSSNLV | |
| | | | GEKPYKCPECGKSFSQSSNLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆QSGD | |
| | | | QRTHTGEKPYKCPECGKSFSQSG | | LRRHX₁₇X₁₈X₁₉HX₂₀X₂₁X₂₂X₂₃X₂₄X₈X₉CX₁₀X₁₁CX₁₂X₁₃X₁₄X₁₅X₁₆QR | |
| | | | DLRRHQRTHTGEKPYKCPECGKS | | AHLERHX₁₇X₁₈X₁₉HX₂₀X₂₁X₂₂X₂₃X₂₄X₂₅X₂₆X₂₇X₂₈X₂₉X₃₀ | |
| | | | FSQRAHLERHQRTHTGEKPTGKK | | | |
| | | | TS | | | |
| gttcccctg | GTT CCC | 1397 | LEPGEKPYKCPECGKSFSRSDHL | 1946 | X₁X₂X₃X₄X₅X₆X₇X₈X₉CX₁₀X₁₁CX₁₂X₁₃X₁₄X₁₅X₁₆RSDHLTTHX₁₇X₁₈X₁₉ | 2495 |
| ggagcagaa | CTG GGA | | TTHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QSSNLVRHX₁₇X₁₈ | |
| tgg | GCA GAA | | QSSNLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QSGDLRRHX₁₇ | |
| | TGG | | GKSFSQSGDLRRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QRAHLERH | |
| | | | KCPECGKSFSQRAHLERHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RNDALT | |
| | | | GEKPYKCPECGKSFSRNDALTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆SKKH | |
| | | | QRTHTGEKPYKCPECGKSFSSKK | | LAEHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | HLAEHQRTHTGEKPYKCPECGKS | | GSLVRHX₁₇X₁₈X₁₉HX₂₀X₂₁X₂₂X₂₃X₂₄X₂₅X₂₆X₂₇X₂₈X₂₉X₃₀ | |
| | | | FSTSGSLVRHQRTHTGEKPTGKK | | | |
| | | | TS | | | |
| ccggttccc | CCG GTT | 1398 | LEPGEKPYKCPECGKSFSQSSNL | 1947 | X₁X₂X₃X₄X₅X₆X₇X₈X₉CX₁₀X₁₁CX₁₂X₁₃X₁₄X₁₅X₁₆QSSNLVRHX₁₇X₁₈X₁₉ | 2496 |
| ctgggagca | CCC CTG | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QSGDLRRHX₁₇X₁₈ | |
| gaa | GGA GCA | | QSGDLRRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QRAHLERHX₁₇ | |
| | GAA | | GKSFSQRAHLERHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RNDALTEH | |
| | | | KCPECGKSFSRNDALTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆SKKHLA | |
| | | | GEKPYKCPECGKSFSSKKHLAEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆TSGS | |
| | | | QRTHTGEKPYKCPECGKSFSTSG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RN | |
| | | | SLVRHQRTHTGEKPYKCPECGKS | | DTLTEHX₁₇X₁₃X₁₉HX₂₀X₂₁X₂₂X₂₃X₂₄X₂₅X₂₆X₂₇X₂₈X₂₉X₃₀ | |
| | | | FSRNDTLTEHQRTHTGEKPTGKK | | | |
| | | | TS | | | |
| tgggagcag | TGG gag | 1399 | LEPGEKPYKCPECGKSFSQSSNL | 1948 | X₁X₂X₃X₄X₅X₆X₇X₈X₉CX₁₀X₁₁CX₁₂X₁₃X₁₄X₁₅X₁₆QSSNLVRHX₁₇X₁₈X₁₉ | 2497 |
| aatggactg | CAG AAT | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RNDALTEHX₁₇X₁₈ | |
| gaa | GGA CTG | | RNDALTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QRAHLERHX₁₇ | |
| | GAA | | GKSFSQRAHLERHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TTGNLTVH | |
| | | | KCPECGKSFSTTGNLTVHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RADNLT | |
| | | | GEKPYKCPECGKSFSRADNLTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆RSDN | |
| | | | QRTHTGEKPYKCPECGKSFSRSD | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | NLVRHQRTHTGEKPYKCPECGKS | | DHLTTHX₁₇X₁₈X₁₉HX₂₀X₂₁X₂₂X₂₃X₂₄X₂₅X₂₆X₂₇X₂₈X₂₉X₃₀ | |
| | | | FSRSDHLTTHQRTHTGEKPTGKK | | | |
| ccctgggag | CCC TGG | 1400 | LEPGEKPYKCPECGKSFSRNDAL | 1949 | X₁X₂X₃X₄X₅X₆X₇X₈X₉CX₁₀X₁₁CX₁₂X₁₃X₁₄X₁₅X₁₆RNDALTEHX₁₇X₁₈X₁₉ | 2498 |
| cagaatgga | gag CAG | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QRAHLERHX₁₇X₁₈ | |
| ctg | AAT GGA | | QRAHLERHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TTGNLTVHX₁₇ | |
| | CTG | | GKSFSTTGNLTVHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RADNLTEH | |
| | | | KCPECGKSFSRADNLTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDNLV | |
| | | | GEKPYKCPECGKSFSRSDNLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDH | |
| | | | QRTHTGEKPYKCPECGKSFSRSD | | LTTHX₁₇X₁₈X₁₉HX₂₀X₂₁X₂₂X₂₃X₂₄X₈X₉CX₁₀X₁₁CX₁₂X₁₃X₁₄X₁₅X₁₆SK | |
| | | | HLTTHQRTHTGEKPYKCPECGKS | | KHLAEHX₁₇X₁₈X₁₉HX₂₀X₂₁X₂₂X₂₃X₂₄X₂₅X₂₆X₂₇X₂₈X₂₉X₃₀ | |
| | | | FSSKKHLAEHQRTHTGEKPTGKK | | | |
| | | | TS | | | |
| ctggaagtt | CTG GAA | 1401 | LEPGEKPYKCPECGKSFSRADNL | 1950 | X₁X₂X₃X₄X₅X₆X₇X₈X₉CX₁₀X₁₁CX₁₂X₁₃X₁₄X₁₅X₁₆RADNLTEHX₁₇X₁₈X₁₉ | 2499 |
| tgggagggc | GTT TGG | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆DPGHLVRHX₁₇X₁₈ | |
| cag | GAG GGC | | DPGHLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDNLVRHX₁₇ | |
| | CAG | | GKSFSRSDNLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDHLTTH | |
| | | | KCPECGKSFSRSDHLTTHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TSGSLV | |
| | | | GEKPYKCPECGKSFSTSGSLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆QSSN | |
| | | | QRTHTGEKPYKCPECGKSFSQSS | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆RN | |
| | | | NLVRHQRTHTGEKPYKCPECGKS | | DALTEHX₁₇X₁₈X₁₉HX₂₀X₂₁X₂₂X₂₃X₂₄X₂₅X₂₆X₂₇X₂₈X₂₉X₃₀ | |
| | | | FSRNDALTEHQRTHTGEKPTGKK | | | |
| | | | TS | | | |
| gaagtttgg | GAA GTT | 1402 | LEPGEKPYKCPECGKSFSHKNAL | 1951 | X₁X₂X₃X₄X₅X₆X₇X₈X₉CX₁₀X₁₁CX₁₂X₁₃X₁₄X₁₅X₁₆HKNALQNHX₁₇X₁₈X₁₉ | 2500 |
| gagggccag | TGG GAG | | QNHQRTHTGEKPYKCPECGKSFS | | HX₂₀X₂₁X₂₂X₂₃X₂₄X₈X₉CX₁₀X₁₁CX₁₂XEX₁₄X₁₅X₁₆RADNLTEHX₁₇X₁₈ | |
| att | GGC CAG | | RADNLTEHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DPGHLVRHX₁₇ | |
| | ATT | | GKSFSDPGHLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RSDNLVRH | |
| | | | KCPECGKSFSRSDNLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDHLT | |
| | | | GEKPYKCPECGXSFSRSDHLTTH | | THX₁₇X₁₈X₁₉HX₂₀X₂₁X₂₂X₂₃X₂₄X₈X₉CX₁₀X₁₁CX₁₂X₁₃X₁₄X₁₅X₁₆TSGS | |
| | | | QRTHTGEKPYKCPECGKSFSTSG | | LVRHX₁₇X₁₈X₁₉HX₂₀X₂₁X₂₂X₂₃X₂₄X₈X₉CX₁₀X₁₁CX₁₂X₁₃X₁₄X₁₅X₁₆QS | |
| | | | SLVRHQRTHTGEKPYKCPECGKS | | SNLVRHX₁₇X₁₈X₁₉HX₂₀X₂₁X₂₂X₂₃X₂₄X₂₅X₂₆X₂₇X₂₈X₂₉X₃₀ | |
| | | | FSQSSNLVRHQRTHTGEKPTGKK | | | |
| | | | TS | | | |
| gtttgggag | GTT TGG | 1403 | LEPGEKPYKCPECGKSFSSKKAL | 1952 | X₁X₂X₃X₄X₅X₆X₇X₈X₉CX₁₀X₁₁CX₁₂X₁₃X₁₄X₁₅X₁₆TSGSLVRHX₁₇X₁₈X₁₉ | 2501 |
| ggccagatt | GAG GGC | | TEHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QSSNLVRHX₁₇X₁₈ | |
| cac | CAG ATT | | HKNALQNHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RNDALTEHX₁₇ | |
| | CAC | | GKSFSRADNLTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QRAHLERH | |
| | | | KCPECGKSFSDPGHLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RSDNLV | |
| | | | GEKPYKCPECGKSFSRSDNLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RSDH | |
| | | | QRTHTGEKPYKCPECGKSFSRSD | | LTTHX₁₇X₁₈X₁₉HX₂₀X₂₁X₂₂X₂₃X₂₄X₈X₉CX₁₀X₁₁CX₁₂X₁₃X₁₄X₁₅X₁₆TS | |
| | | | HLTTHQRTHTGEKPYKCPECGKS | | GSLVRHX₁₇X₁₈X₁₉HX₂₀X₂₁X₂₂X₂₃X₂₄X₂₅X₂₆X₂₇X₂₈X₂₉X₃₀ | |
| | | | FSTSGSLVRHQRTHTGEKPTGKK | | | |
| | | | TS | | | |
| gagcagaat | gag CAG | 1404 | LEPGEKPYKCPECGKSFSTSGSL | 1953 | X₁X₂X₃X₄X₅X₆X₇X₈X₉CX₁₀X₁₁CX₁₂X₁₃X₁₄X₁₅X₁₆TSGSLVRHX₁₇X₁₈X₁₉ | 2502 |
| ggactggaa | AAT GGA | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆QSSNLVRHX₁₇X₁₈ | |
| gtt | CTG GAA | | QSSNLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RNDALTEHX₁₇ | |
| | GTT | | GKSFSRNDALTEHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QRAHLERH | |
| | | | KCPECGKSFSQRAHLERHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆TTGNLT | |
| | | | GEKPYKCPECGKSFSTTGNLTVH | | VHX₁₇X₁₈X₁₉HX₂₀X₂₁X₂₂X₂₃X₂₄X₈X₉CX₁₀X₁₁CX₁₂X₁₃X₁₄X₁₅X₁₆RADN | |
| | | | QRTHTGEKPYKCPECGKSFSRAD | | LTEHX₁₇X₁₈X₁₉HX₂₀X₂₁X₂₂X₂₃X₂₄X₈X₉CX₁₀X₁₁CX₁₂X₁₃X₁₄X₁₅X₁₆RS | |
| | | | NLTEHQRTHTGEKPYKCPECGKS | | DNLVRHX₁₇X₁₈X₁₉HX₂₀X₂₁X₂₂X₂₃X₂₄X₂₅X₂₆X₂₇X₂₈X₂₉X₃₀ | |
| | | | FSRSDNLVRHQRTHTGEKPTGKK | | | |
| | | | TS | | | |
| aatggactg | AAT GGA | 1405 | LEPGEKPYKCPECGKSFSRSDNL | 1954 | X₁X₂X₃X₄X₅X₆X₇X₈X₉CX₁₀X₁₁CX₁₂X₁₃X₁₄X₁₅X₁₆RSDNLVRHX₁₇X₁₈X₁₉ | 2503 |
| gaagtttgg | CTG GAA | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDHLTTHX₁₇X₁₈ | |
| gag | GTT TGG | | RSDHLTTHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆TSGSLVRHX₁₇ | |
| | GAG | | GKSFSTSGSLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QSSNLVRH | |
| | | | KCPECGKSFSQSSNLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆RNDALT | |
| | | | GEKPYKCPECGKSFSRNDALTEH | | EHX₁₇X₁₈X₁₉HX₂₀X₂₁X₂₂X₂₃X₂₄X₈X₉CX₁₀X₁₁CX₁₂X₁₃X₁₄X₁₅X₁₆QRAH | |
| | | | QRTHTGEKPYKCPECGKSFSQRA | | LERHX₁₇X₁₈X₁₉HX₂₀X₂₁X₂₂X₂₃X₂₄X₈X₉CX₁₀X₁₁CX₁₂X₁₃X₁₄X₁₅X₁₆TT | |
| | | | HLERHQRTHTGEKPYKCPECGKS | | GNLTVHX₁₇X₁₈X₁₉HX₂₀X₂₁X₂₂X₂₃X₂₄X₂₅X₂₆X₂₇X₂₈X₂₉X₃₀ | |
| | | | FSTTGNLTVHQRTHTGEKPTGKK | | | |
| | | | TS | | | |
| ggactggaa | GGA CTG | 1406 | LEPGEKPYKCPECGKSFSDPGHL | 1955 | X₁X₂X₃X₄X₅X₆X₇X₈X₉CX₁₀X₁₁CX₁₂X₁₃X₁₄X₁₅X₁₆DPGHLVRHX₁₇X₁₈X₁₉ | 2504 |
| gtttgggag | GAA GTT | | VRHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDNLVRHX₁₇X₁₈ | |
| ggc | TGG GAG | | RSDNLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RSDHLTTHX₁₇ | |
| | GGC | | GKSFSRSDHLTTHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆TSGSLVRH | |
| | | | KCPECGKSFSTSGSLVRHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QSSNLV | |
| | | | GEKPYKCPECGKSFSQSSNLVRH | | RHX₁₇X₁₈X₁₉HX₂₀X₂₁X₂₂X₂₃X₂₄X₈X₉CX₁₀X₁₁CX₁₂X₁₃X₁₄X₁₅X₁₆RNDA | |
| | | | QRTHTGEKPYKCPECGKSFSRND | | LTEHX₁₇X₁₈X₁₉HX₂₀X₂₁X₂₂X₂₃X₂₄X₈X₉CX₁₀X₁₁CX₁₂XEX₁₄X₁₅X₁₆QR | |
| | | | ALTEHQRTHTGEKPYKCPECGKS | | AHLERHX₁₇X₁₈X₁₉HX₂₀X₂₁X₂₂X₂₃X₂₄X₂₅X₂₆X₂₇X₂₈X₂₉X₃₀ | |
| | | | FSQRAHLERHQRTHTGEKPTGKK | | | |
| | | | TS | | | |
| cagaatgga | CAG AAT | 1407 | LEPGEKPYKCPECGKSFSRSDHL | 1956 | X₁X₂X₃X₄X₅X₆X₇X₈X₉CX₁₀X₁₁CX₁₂X₁₃X₁₄X₁₅X₁₆RSDHLTTHX₁₇X₁₈X₁₉ | 2505 |
| ctggaagtt | GGA CTG | | TTHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆TSGSLVRHX₁₇X₁₈ | |
| tgg | GAA GTT | | TSGSLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆QSSNLVRHX₁₇ | |
| | TGG | | GKSFSQSSNLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RNDALTEH | |
| | | | KCPECGKSFSRNDALTEHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QRAHLE | |
| | | | GEKPYKCPECGKSFSQRAHLERH | | RHX₁₇X₁₈X₁₉HX₂₀X₂₁X₂₂X₂₃X₂₄X₈X₉CX₁₀X₁₁CX₁₂X₁₃X₁₄X₁₅X₁₆TTGN | |
| | | | QRTHTGEKPYKCPECGKSFSTTG | | LTVHX₁₇X₁₈X₁₉HX₂₀X₂₁X₂₂X₂₃X₂₄X₈X₉CX₁₀X₁₁CX₁₂X₁₃X₁₄X₁₅X₁₆RA | |
| | | | NLTVHQRTHTGEKPYKCPECGKS | | DNLTEHX₁₇X₁₈X₁₉HX₂₀X₂₁X₂₂X₂₃X₂₄X₂₅X₂₆X₂₇X₂₈X₂₉X₃₀ | |
| | | | FSRADNLTEHQRTHTGEKPTGKK | | | |
| | | | TS | | | |
| cctgggagc | CCT ggg | 1408 | LEPGEKPYKCPECGKSFSRSDHL | 1957 | X₁X₂X₃X₄X₅X₆X₇X₈X₉CX₁₀X₁₁CX₁₂X₁₃X₁₄X₁₅X₁₆RSDHLTTHX₁₇X₁₈X₁₉ | 2506 |
| agaatggac | AGC AGA | | TTHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆DPGNLVRHX₁₇X₁₈ | |
| tgg | ATG GAC | | DPGNLVRHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆RRDELNVHX₁₇ | |
| | TGG | | GKSFSRRDELNVHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆QLAHLRAH | |
| | | | KCPECGKSFSQLAHLRAHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆ERSHLR | |
| | | | GEKPYKCPECGKSFSERSHLREH | | EHX₁₇X₁₈X₁₉HX₂₀X₂₁X₂₂X₂₃X₂₄X₈X₉CX₁₀X₁₁CX₁₂X₁₃X₁₄X₁₅X₁₆RSDK | |
| | | | QRTHTGEKPYKCPECGKSFSRSD | | LVRHX₁₇X₁₈X₁₉HX₂₀X₂₁X₂₂X₂₃X₂₄K₈X₉CX₁₀X₁₁CX₁₂X₁₃X₁₄X₁₅X₁₆TK | |
| | | | KLVRHQRTHTGEKPYKCPECGKS | | NSLTEHX₁₇X₁₈X₁₉HX₂₀X₂₁X₂₂X₂₃X₂₄X₂₅X₂₆X₂₇X₂₈X₂₉X₃₀ | |
| | | | FSTKNSLTEHQRTHTGEKPTGKK | | | |
| | | | TS | | | |
| gggagcaga | ggg AGC | 1409 | LEPGEKPYKCPECGKSFSRKDNL | 1958 | X₁X₂X₃X₄X₅X₆X₇X₈X₉CX₁₀X₁₁CX₁₂X₁₃X₁₄X₁₅X₁₆RKDNLKNHX₁₇X₁₈X₁₉ | 2507 |
| atggactgg | AGA ATG | | KNHQRTHTGEKPYKCPECGKSFS | | HX₂₀X₂₁X₂₂X₂₃X₂₄X₈X₉CX₁₀X₁₁CX₁₂X₁₃X₁₄X₁₅X₁₆RSDHLTTHX₁₇X₁₈ | |
| aag | GAC TGG | | RSDHLTTHQRTHTGEKPYKCPEC | | X₁₉HX₂₀X₂₁X₂₂X₂₃X₂₄X₈X₉CX₁₀X₁₁CX₁₂X₁₃X₁₄X₁₅X₁₆DPGNLVRHX₁₇ | |
| | AAG | | GKSFSDPGNLVRHQRTHTGEKPY | | X₁₈X₁₉HX₂₀X₂₁X₂₂X₂₃X₂₄X₈X₉CX₁₀X₁₁CX₁₂X₁₃X₁₄X₁₅X₁₆RRDELNVH | |
| | | | KCPECGKSFSRRDELNVHQRTHT | | X₁₇X₁₈X₁₉HX₂₀X₂₁X₂₂X₂₃X₂₄X₈X₉CX₁₀X₁₁CX₁₂X₁₃X₁₄X₁₅X₁₆QLAHLR | |
| | | | GEKPYKCPECGKSFSQLAHLRAH | | AHX₁₇X₁₈X₁₉HX₂₀X₂₁X₂₂X₂₃X₂₄X₈X₉CX₁₀X₁₁CX₁₂X₁₃X₁₄X₁₅X₁₆ERSH | |
| | | | QRTHTGEKPYKCPECGKSFSERS | | LREHX₁₇X₁₈X₁₉HX₂₀X₂₁X₂₂X₂₃X₂₄X₈X₉CX₁₀K₁₁CX₁₂X₁₃X₁₄X₁₅X₁₆RS | |
| | | | HLREHQRTHTGEKPYKCPECGKS | | DKLVRHX₁₇X₁₈X₁₉HX₂₀X₂₁X₂₂X₂₃X₂₄X₂₅X₂₆X₂₇X₂₈X₂₉X₃₀ | |
| | | | FSRSDKLVRHQRTHTGEKPTGKK | | | |
| | | | TS | | | |

### EQUIVALENTS

Those skilled in the art will recognize, or be able to ascertain using no more than routine experimentation, many equivalents to the specific embodiments of the present invention described herein. Such equivalents are intended to be encompassed by the following claims.

1. A site-specific hepatocyte nuclear factor 4a (HNF4α) disrupting agent, comprising a site-specific HNF4α targeting moiety which targets an HNF4α expression control region, and wherein the expression control region comprises the nucleotide sequence of HNF4α promoter 1, or a fragment thereof.

2. The site-specific HNF4α disrupting agent of claim 1, wherein the expression control region comprises the nucleotide sequence of any one of the nucleotide sequences selected from the group consisting of SEQ ID NOs: 102, 106, 110, 114, 118, 122, 126, 130, 134, 138, 142, 146, 150, 154, and 158, or wherein the expression control region comprises the nucleotide sequence of any one of the nucleotide sequences listed in column 1 of Table 10.

3. The site-specific HNF4α disrupting agent of claim 1, wherein the HNF4α targeting moiety comprises (i) a DNA-binding domain of a zinc finger (ZNF) polypeptide, or a fragment thereof, or (ii) a DNA-binding domain of a transcription activator-like effector (TALE) polypeptide, or a fragment thereof, that specifically binds to the HNF4α expression control region.

4. The site-specific HNF4α disrupting agent of claim 1, wherein the HNF4α targeting moiety comprises the DNA-binding domain of a zinc finger (ZNF) polypeptide, or a fragment thereof, that specifically binds to the HNF4α expression control region.

5. The site-specific HNF4α disrupting agent of claim 4, wherein the DNA-binding domain of the ZNF polypeptide comprises an amino acid sequence having at least 85%, 90% or 95% amino acid identity to the entire amino acid sequence of any one of the amino acid sequences selected from the group consisting of SEQ ID NOs: 103, 107, 111, 115, 119, 123, 127, 131, 135, 139, 143, 147, 151, 155, and 159.

6. The site-specific HNF4α disrupting agent of claim 1, wherein the HNF4α targeting moiety comprises the DNA-binding domain of a transcription activator-like effector (TALE) polypeptide, or a fragment thereof, that specifically binds to the HNF4α expression control region.

7. The site-specific HNF4α disrupting agent of claim 6, wherein the DNA-binding domain of the TALE polypeptide comprises an amino acid sequence having at least 85%, 90% or 95% amino acid identity to the entire amino acid sequence of any one of the amino acid sequences selected from the group consisting of SEQ ID NOs: 186, 189, 192, 195, 198, 201, 204, 207, and 210.

8. The site-specific HNF4α disrupting agent of claim 3, wherein the HNF4α disrupting agent further comprises an effector molecule.

9. The site-specific HNF4α disrupting agent of claim 8, wherein the effector molecule is fused to the DNA-binding domain of the ZNF polypeptide or the TALE polypeptide.

10. The site-specific HNF4α disrupting agent of claim 8, wherein the effector molecule is selected from the group consisting of a physical blocker, an epigenetic recruiter, and an epigenetic CpG modifier.

11. The site-specific HNF4α disrupting agent of claim 10, wherein the epigenetic recruiter is selected from the group consisting of a transcriptional enhancer and a transcriptional repressor; and/or
wherein the CpG modifier is selected from the group consisting of a DNA methylase, a DNA demethylase, a histone modifying agent, and a histone deacetylase.

12. The site-specific HNF4α disrupting agent of claim 9, wherein the effector molecule is VPR (VP64-p65-Rta) or P300.

13. A nucleic acid molecule encoding the site-specific HNF4α disrupting agent of claim 1.

14. The nucleic acid molecule of claim 13, wherein the nucleic acid molecule is formulated in a lipid nanoparticle (LNP).

15. A cell comprising the site-specific HNF4α disrupting agent of claim 1.

16. A method of modulating expression of hepatocyte nuclear factor 4 alpha (HNF4α) in a cell, the method comprising contacting the cell with a site-specific HNF4α disrupting agent of claim 1, or a nucleic acid molecule encoding the site-specific HNF4α disrupting agent of claim 1, thereby modulating expression of HNF4α in the cell.

17. The method of claim 16, wherein the cell is within a subject, and wherein the subject has an HNF4α-associated disease.

18. The method of claim 17, wherein the HNF4α-associated disease is selected from the group consisting of fatty liver (steatosis), nonalcoholic steatohepatitis (NASH), cirrhosis of the liver, accumulation of fat in the liver, inflammation of the liver, hepatocellular necrosis, liver fibrosis, and nonalcoholic fatty liver disease (NAFLD), polycystic kidney disease, inflammatory bowel disease (IBD), and MODY I.

19. A method for treating a subject having an HNF4α-associated disease, comprising administering to the subject a therapeutically effective amount of the site-specific HNF4α disrupting agent of claim 1, or a nucleic acid molecule encoding the site-specific HNF4α disrupting agent of claim 1, thereby treating the subject.

20. The method of claim 19, wherein the HNF4α-associated disease is selected from the group consisting of fatty liver (steatosis), nonalcoholic steatohepatitis (NASH), cirrhosis of the liver, accumulation of fat in the liver, inflammation of the liver, hepatocellular necrosis, liver fibrosis, and nonalcoholic fatty liver disease (NAFLD) and the site-specific HNF4α disrupting agent enhances expression of HNF4α in the subject.
